(19) United States
(12) Patent Application Publication
(10) Pub. No.: US 2021/0214374 A1
(43) Pub. Date: Jul. 15, 2021

(54) COMPOUNDS AND COMPOSITIONS FOR OCULAR DELIVERY

(71) Applicant: Graybug Vision, Inc., Redwood City, CA (US)

(72) Inventors: John G. Bauman, El Sobrante, CA (US); Ming Yang, Lutherville-Timonium, MD (US); Nu Hoang, Annapolis, MD (US); Emmett Cunningham, Hillsborough, CA (US); Jeffrey L. Cleland, San Carlos, CA (US)

(73) Assignee: Graybug Vision, Inc., Redwood City, CA (US)

(21) Appl. No.: 17/212,873

(22) Filed: Mar. 25, 2021

Related U.S. Application Data
(63) Continuation of application No. PCT/US2019/053513, filed on Sep. 27, 2019.
(60) Provisional application No. 62/737,678, filed on Sep. 27, 2018.

(51) Int. Cl. C07D 513/04; C07D 495/04; A61K 9/50; C07D 403/06

(52) U.S. Cl. CPC C07D 513/04; C07D 403/06; A61K 9/5031; C07D 495/04

(57) ABSTRACT

The present invention provides new prodrugs of Sunitinib, Brinzolamide, and Dorzolamide and compositions to treat medical disorders, for example glaucoma, a disorder or abnormality related to an increase in intraocular pressure (IOP), a disorder requiring neuroprotection, age-related macular degeneration, or diabetic retinopathy.

### CROSS-REFERENCE TO RELATED APPLICATIONS

This application is a continuation of International Patent Application No. PCT/US2019/053513 filed in the U.S. Receiving Office on Sep. 27, 2019, which claims priority to U.S. Provisional Application No. 62/737,678, filed Sep. 27, 2018. The entirety of each of these applications is incorporated herein by reference.

### BACKGROUND

The eye is a complex organ with unique anatomy and physiology. The structure of the eye can be divided into two parts, the anterior and posterior. The cornea, conjunctiva, aqueous humor, iris, ciliary body and lens are in the anterior portion. The posterior portion includes the sclera, choroid, retinal pigment epithelium, neural retina, optic nerve and vitreous humor. The most prevalent diseases affecting the posterior segment of the eye are dry and wet age-related macular degeneration (AMD) and diabetic retinopathy. The most important diseases affecting the anterior segment include glaucoma, allergic conjunctivitis, anterior uveitis and cataracts. Glaucoma, which damages the eye's optic nerve, is a leading cause of vision loss and blindness.

To address issues of ocular delivery, a large number of types of delivery systems have been devised, including conventional (solution, suspension, emulsion, ointment, inserts and gels); vesicular (liposomes, exosomes, niosomes, discomes and pharmacosomes); advanced materials (scleral plugs, gene delivery, siRNA and stem cells); and, controlled release systems (implants, hydrogels, dendrimers, iontophoresis, collagen shields, polymeric solutions, therapeutic contact lenses, cyclodextrin carriers, microneedles and microemulsions and particulates (microparticles and nanoparticles)).

Topical drops are widely used non-invasive routes of drug administration to treat anterior ocular diseases due to their non-invasiveness and convenience. Typical routes of drug delivery to the eye are topical, systemic, subconjunctival, intravitreal, punctal, intrasceral, transscleral, anterior or posterior sub-Tenon's, suprachoroidal, choroidal, subchoroidal, and subretinal.

Drug delivery to the posterior area of the eye usually requires a different mode of administration from topical drops, and is typically achieved via an intravitreal injection, periocular injection or systemic administration. Systemic administration is not preferred given the ratio of volume of the eye to the entire body and thus unnecessary potential systemic toxicity. Therefore, intravitreal injections are currently the most common form of drug administration for posterior disorders. However, intravitreal injections are also associated with risk due to the common side effect of inflammation to the eye caused by administration of foreign material to this sensitive area, endophthalmitis, hemorrhage, retinal detachment and poor patient compliance.

Transscleral delivery with periocular administration is seen as an alternative to intravitreal injections, however, ocular barriers such as the sclera, choroid, retinal pigment epithelium, lymphatic flow and general blood flow compromise efficacy.

To treat ocular diseases, and in particular disease of the posterior chamber, the drug must be delivered in an amount and for a duration to achieve efficacy.

Examples of common drug classes used for ocular disorders include: prostaglandins, carbonic anhydrase inhibitors, receptor tyrosine kinase inhibitors (RTKIs), Rho kinase (ROCK) inhibitors, beta-blockers, alpha-adrenergic agonists, parasympathomimetics, epinephrine, and hyperosmotic agents.

Patent applications that describe anhydrase inhibitors (CAIs) include PCT Application Nos. WO 2008/075155 assigned to Nicox S.A.; WO 2014/190763 assigned to Jenkem Technology Co.; WO 2008/132114 assigned to Duke Chem, S.A.; and, WO 2011/163594 assigned to Alkermes. Granted U.S. Patents include U.S. Pat. Nos. 5,120,757 and 5,441,722 assigned to Merck & Co.; U.S. Pat. No. 7,030,250 assigned to Ragatives, S.I.; and, U.S. Pat. No. 8,592,427 assigned to Alkermes.

Johns Hopkins University has filed a number of patents claiming formulations for ocular injections including WO2013/138343 titled “Controlled Release Formulations for the Delivery of HIF-1 Inhibitors”, WO2013/138346 titled “Non-linear Multiblock Copolymer-drug Conjugates for the Delivery of Active Agents”, WO2011/106702 titled “Sustained Delivery of Therapeutic Agents to an Eye Compartment”, WO2016/025215 titled “Glucorticoid-loaded Nanoparticles for Prevention of Corneal Allograft Rejection and Neovascularization”, WO2016/100392 titled “Sunitinib Formulations and Methods for Use Thereof in Treatment of Ocular Disorders”, WO2016/100380 titled “Sunitinib Formulation and Methods for Use Thereof in Treatment of Glaucoma”, WO2016/118506 titled “Compositions for the Sustained Release of Anti-Glaucoma Agents to Control Intraocular Pressure”, WO2013/166385 titled “Nanocrystals, Compositions, and Methods that Aid Particle Transport in Mucus”, WO2005/072710 titled “Drug and Gene Carrier Particles that Rapidly move Through Mucus Barriers,” WO2008/030557 titled “Compositions and Methods for Enhancing Transport through Mucus”, WO2012/061703 titled “Compositions and Methods Relating to Reduced Mucoadhesion,” WO2012/039979 titled “Large Nanoparticles that Penetrate Tissue,” WO2012/109363 titled “Mucus Penetrating Gene Carriers”, WO2013/090804 titled “Biodegradable Stealth Nanoparticles Prepared by a Novel Self-Assembly Emulsification Method,” WO2013/110028 titled “Nanoparticles Formulations with Enhanced Mucosal Penetration”, and WO2013/166498 titled “Lipid-based Drug Carriers for Rapid Penetration through Mucus Linings”.

GrayBug Vision, Inc. discloses prodrugs for the treatment of ocular therapy in granted U.S. Pat. Nos. 9,808,531; 9,956,302; 10,098,965; 10,111,964; 10,117,950; and 10,159,747; U.S. Application No. 2019-0060474; and PCT Application Nos. WO 2017/053638; WO 2018/175922; and WO 2019/118924. Aggregating microparticles for ocular therapy are described in US 2017-0135960, WO 2017/083779, US 2018-0326078, and WO 2018/209155.

Despite research, there still is a need to deliver effective therapies to the eye that reduce ocular pressure. Therefore, the object of this invention is to provide additional compounds, compositions and methods to treat ocular disorders.

### SUMMARY

The present invention provides new prodrugs, including oligomeric prodrugs, and compositions thereof of Sunitinib, Brinzolamide, or Dorzolamide to provide therapies that are advantageous for ocular delivery.

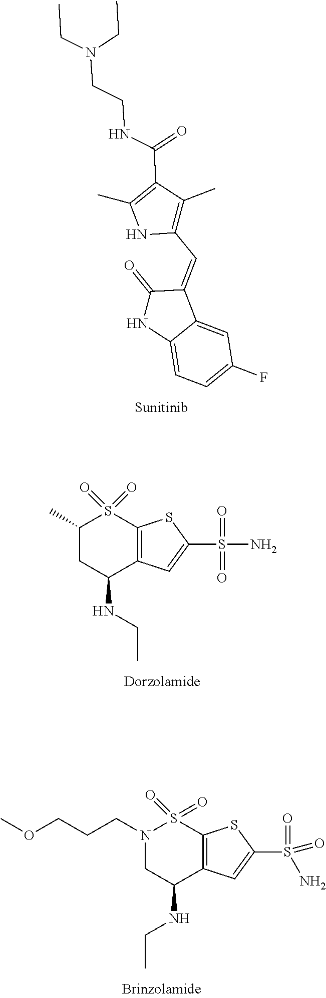 [SMILES: CCN(CC)CCNC(=O)c1c(C)[nH]c(/C=C2\C(=O)Nc3ccc(F)cc32)c1C ; CCN[C@H]1C[C@H](C)S(=O)(=O)c2sc(S(N)(=O)=O)cc21 ; CCN[C@H]1CN(CCCOC)S(=O)(=O)c2sc(S(N)(=O)=O)cc21]

In one embodiment, the invention is an active compound or pharmaceutically acceptable salt of Formula I, Formula II, Formula III, Formula IV, Formula V, Formula VI, Formula VII, Formula VIII, Formula IX, Formula X, Formula XI, Formula XII, Formula XIII, Formula XIV, or Formula XV. In one embodiment, the invention is a method for delivering an active prodrug to the eye that includes presenting it as discussed herein in a controlled delivery system, for example a microparticle or nanoparticle, that allows for sustained delivery.

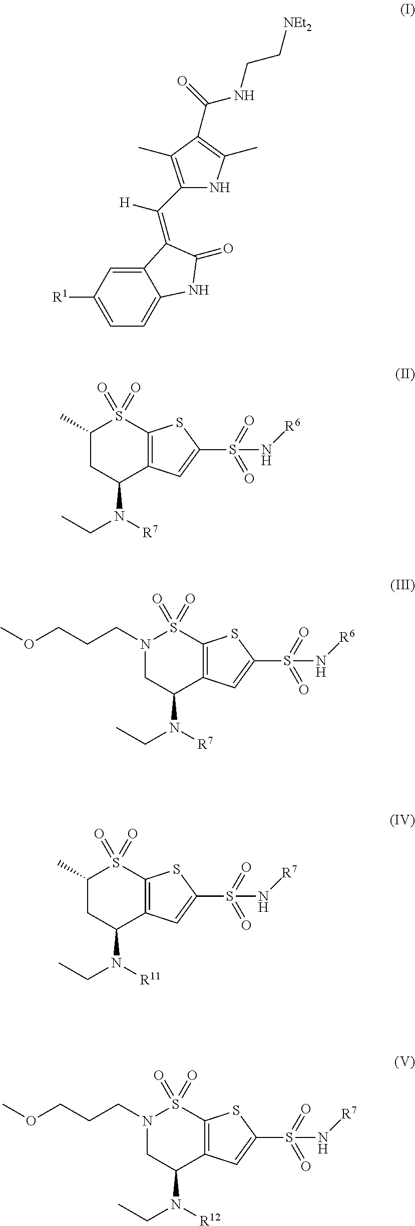

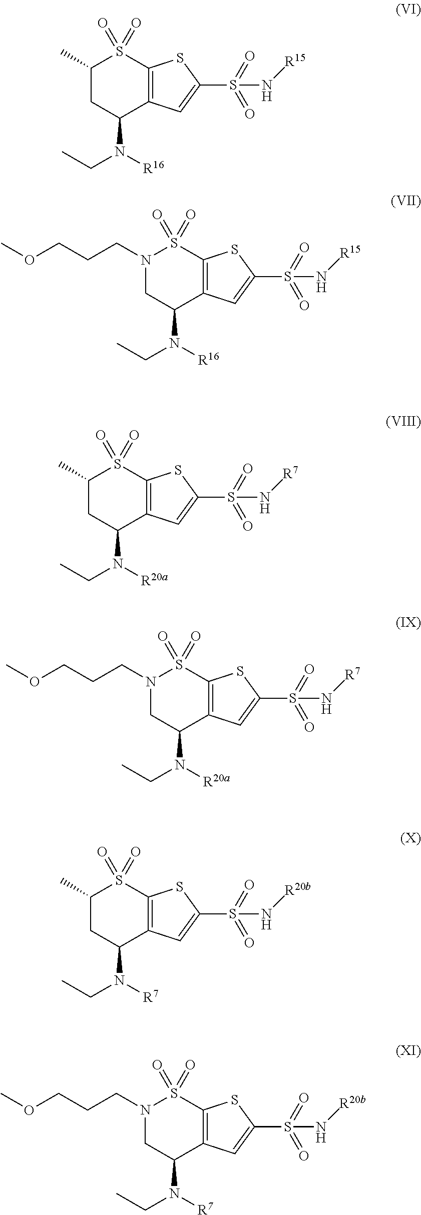

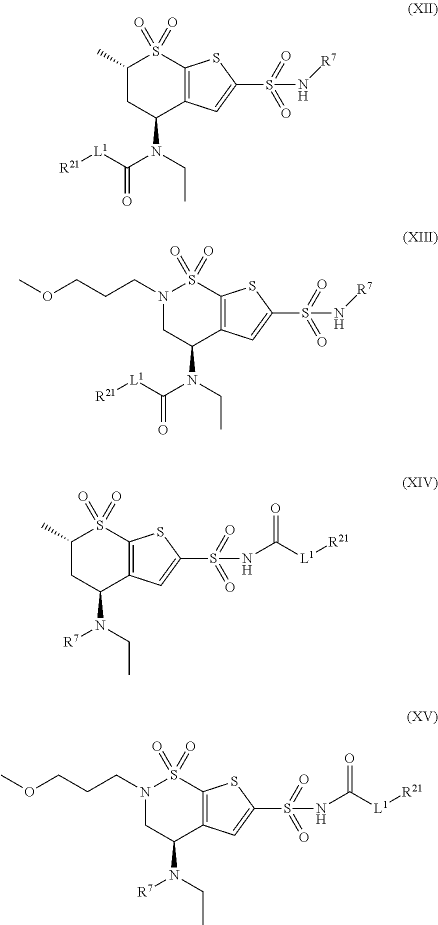

The active therapeutic agent delivered in modified form is selected from Sunitinib, Brinzolamide, and Dorzolamide.

In one embodiment, a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or a pharmaceutically acceptable salt or composition thereof, is administered to a patient in need thereof for the treatment of an ocular disorder. The decreased rate of release of the active material to the ocular compartment may result in decreased inflammation, which has been a significant side effect of ocular therapy to date.

In one embodiment, the compound or a pharmaceutically acceptable salt thereof is provided to the patient by administration to the eye via intravitreal, intrastromal, intracameral, sub-tenon, sub-retinal, retro-bulbar, peribulbar, suprachoroidal, choroidal, subchoroidal, conjunctival, episcleral, posterior juxtascleral, circumcorneal, or tear duct injection in combination with one or more pharmaceutically acceptable carriers.

The compounds of the invention can be used for the controlled administration of active compounds to the eye, over a period of at least two, three, four, five or six months or more in a manner that maintains at least a concentration in the eye that is effective for the disorder to be treated.

In one embodiment, the compound or a pharmaceutically acceptable salt thereof is provided in an immediate or controlled delivery system as desired to achieve the appropriate effect. In some embodiments, the prodrug is provided in a microparticle, microcapsule, vesicle, reservoir, or nanoparticle. In one embodiment, the drug is administered in a polymeric formulation that provides a controlled release that is linear. In another embodiment, the release is not linear; however, even the lowest concentration of release over the designated time period is at or above a therapeutically effective dose. In one embodiment, this is achieved by formulating a hydrophobic prodrug of the invention in a polymeric delivery material such as a polymer or copolymer that includes moieties of at least lactic acid, glycolic acid, propylene oxide or ethylene oxide. In a particular embodiment, the polymeric delivery system includes PLGA, PLA or PGA with or without covalently attached or admixed polyethylene glycol. For example, the hydrophobic drug may be delivered in a mixture of PLGA and PLGA-PEG, PEG, PLA, or PLA-PEG. The hydrophobic drug may be delivered in a mixture of PLA and PLGA-PEG, PEG, PLGA, or PLA-PEG.

In certain embodiments, the prodrug of the present invention is delivered in a microparticle or nanoparticle that is a blend of two polymers, for example (i) a PLGA polymer or PLA polymer as described herein and (ii) a PLGA-PEG or PLA-PEG copolymer. In another embodiment, the microparticle or nanoparticle is a blend of three polymers, such as, for example, (i) a PLGA polymer; (ii) a PLA polymer; and, (iii) a copolymer of PLGA-PEG or PLA-PEG. In an additional embodiment, the microparticle or nanoparticle is a blend of (i) a PLA polymer; (ii) a PLGA polymer; (iii) a PLGA polymer that has a different ratio of lactide and glycolide monomers than the PLGA in (ii); and, (iv) a PLGA-PEG or PLA-PEG copolymer. Any ratio of lactide and glycolide in the PLGA can be used that achieves the desired therapeutic effect. In certain illustrative non-limiting embodiments, the ratio of PLA to PLGA by weight in a polymer blend as described is 77/22, 69/30, 49/50, 54/45, 59/40, 64/35, 69/30, 74/25, 79/20, 84/15, 89/10, 94/5, or 99/1.

In certain embodiments, a blend of three polymers that has (i) PLA (ii) PLGA (iii) PLGA with a different ratio of lactide and glycolide monomers than PLGA in (ii) wherein the ratio by weight is 74/20/5 by weight, 69/20/10 by weight, 69/25/5 by weight, or 64/20/15 by weight. In certain embodiments, the PLGA in (ii) has a ratio of lactide to glycolide of 85/15, 75/25, or 50/50. In certain embodiments the PLGA in (iii) has a ratio of lactide to glycolide of 85/15, 75/25, or 50/50.

In certain aspects, the drug may be delivered in a blend of PLGA or PLA and PEG-PLGA, including but not limited to (i) PLGA+approximately by weight 1% PEG-PLGA or (ii) PLA+approximately by weight 1% PEG-PLGA. In certain aspects, the drug may be delivered in a blend of (iii) PLGA/PLA+approximately by weight 1% PEG-PLGA. In certain embodiments, the blend of PLA, PLGA, or PLA/PGA with PLGA-PEG contains approximately from about 0.5% to about 10% by weight of a PEG-PLGA, from about 0.5% to about 5% by weight of a PEG-PLGA, from about 0.5% to about 4% by weight of a PEG-PLGA, from about 0.5% to about 3% by weight of a PEG-PLGA, from about 1.0% to about 3.0% by weight of a PEG-PLGA, from about 0.1% to about 10% of a PEG-PLGA, from about 0.1% to about 5% of a PEG-PLGA, from about 0.1% to about 1% PEG-PLGA, or from about 0.1% to about 2% PEG-PLGA.

In certain non-limiting embodiments, the ratio by weight percent of PLGA to PEG-PLGA in a two polymer blend as described is in the range of about or between the ranges of 40/1, 45/1, 50/1, 55/1, 60/1, 65/1, 70/1, 75/1, 80/1, 85/1, 90/1, 95/1, 96/1, 97/1, 98/1, 99/1. The PLGA can be acid or ester capped. In non-limiting aspects, the drug can be delivered in a two polymer blend of PLGA75:25 4A+approximately 1% PEG-PLGA50:50; PLGA85:15 5A+approximately 1% PEG-PLGA5050; PLGA75:25 6E+approximately 1% PEG-PLGA50:50; or, PLGA50:50 2A+approximately 1% PEG-PLGA50:50.

In certain non-limiting embodiments, the ratio by weight percent of PLA/PLGA-PEG in a polymer blend as described is in the range of about or between the ranges of 40/1, 45/1, 50/1, 55/1, 60/1, 65/1, 70/1, 75/1, 80/1, 85/1, 90/1, 95/1, 96/1, 97/1, 98/1, 99/1. The PLA can be acid capped or ester capped. In certain aspects, the PLA is PLA 4.5A. In non-limiting aspects, the drug is delivered in a blend of PLA 4.5A+1% PEG-PLGA.

The PEG segment of the PEG-PLGA may have, for example, in non-limiting embodiments, a molecular weight of at least about or between 1 kDa, 2 kDa, 3 kDa, 4 kDa, 5 kDa, 6 kDa, 7 kDa, 8 kDa, 9 kDa, or 10 kDa, and typically not greater than 10 kDa, 15 kDa, 20 kDa, or 50 kDa, or in some embodiments, 6 kDa, 7 kDa, 8 kDa, or 9 kDa. In certain embodiment, the PEG segment of the PEG-PLGA has a molecular weight between about 3 kDa and about 7 kDa or between about 2 kDa and about 7 kDa. Non-limiting examples of the PLGA segment of the PEG-PLGA is PLGA50:50, PLGA75:25, or PLGA85:15. In one embodiment, the PEG-PLGA segment is PEG (5 kDa)-PLGA50:50.

When the drug is delivered in a blend of PLGA+PEG-PLGA, any ratio of lactide and glycolide in the PLGA or the PLGA-PEG can be used that achieves the desired therapeutic effect. Non-limiting illustrative embodiments of the ratio of lactide/glycolide in the PLGA or PLGA-PEG are in the range of about or between the ranges of 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. In one embodiment, the PLGA is a block co-polymer, for example, diblock, triblock, multiblock, or star-shaped block. In one embodiment, the PLGA is a random co-polymer. In certain aspects, the PLGA is PLGA75:25 4A; PLGA85:15 5A; PLGA75:25 6E; or, PLGA50:50 2A.

In another embodiment, the polymer includes a polyethylene oxide (PEO) or polypropylene oxide (PPO). In certain aspects, the polymer can be a random, block, diblock, triblock or multiblock copolymer (for example, a polylactide, a polylactide-co-glycolide, polyglycolide or Pluronic). For injection into the eye, the polymer is pharmaceutically acceptable and typically biodegradable so that it does not have to be removed.

It is also important that the decreased rate of release of the drug while maintaining efficacy over an extended time of up to 2, 3, 4, 5 or 6 months be achieved using a particle that is small enough for administration through a needle without causing significant damage or discomfort to the eye and not to give the illusion to the patient of black spots floating in the eye. This typically means the controlled release particle should be less than approximately 300, 250, 200, 150, 100, 50, 45, 40, 35, or 30 μm, such as less than approximately 30, 29, 28, 27, 26, 25, 24, 23, 22 21, or 20 μm. In one aspect, the particles do not agglomerate in vivo to form larger particles, but instead in general maintain their administered size and decrease in size over time.

The hydrophobicity of the conjugated drug can be measured using a partition coefficient (P; such as Log P in octanol/water), or distribution coefficient (D; such as Log D in octanol/water) according to methods well known to those of skill in the art. Log P is typically used for compounds that are substantially un-ionized in water and Log D is typically used to evaluate compounds that ionize in water. In certain embodiments, the conjugated derivatized drug has a Log P or Log D of greater than approximately 2.5, 3, 3.5, 4, 4.5, 5, 5.5 or 6. In other embodiments, the conjugated derivatized drug has a Log P or Log D which is at least approximately 1, 1.5, 2, 2.5, 3, 3.5 or 4 Log P or Log D units, respectively, higher than the parent hydrophilic drug.

This invention includes an active compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or a pharmaceutically acceptable salt or composition thereof. These compounds can be used to treat an ocular disorder in a host, for example a human, in need thereof In one embodiment, an active compound or its salt or composition, as described herein, is used to treat a medical disorder which is glaucoma, a disorder mediated by carbonic anhydrase, a disorder mediated by VEGF, a disorder or abnormality related to an increase in intraocular pressure (IOP), a disorder mediated by nitric oxide synthase (NOS), or a disorder requiring neuroprotection such as to regenerate/repair optic nerves. In another embodiment more generally, the disorder treated is allergic conjunctivitis, anterior uveitis, cataracts, dry or wet age-related macular degeneration (AMD), neovascular age-related macular degeneration (NVAMD), geographic atrophy, or diabetic retinopathy. In one embodiment, an active compound or its salt or composition, as described herein, is used to decrease IOP. In one embodiment, an active compound or its salt or composition is used to treat optic nerve damage associated with IOP.

In other embodiments, Compound 1-1, Compound 2-1, Compound 3-1, Compound 16-2, Compound 25-1, or Compound 26-1 or a pharmaceutically acceptable salt thereof is provided in an effective amount to the patient in a microparticle for ocular delivery.

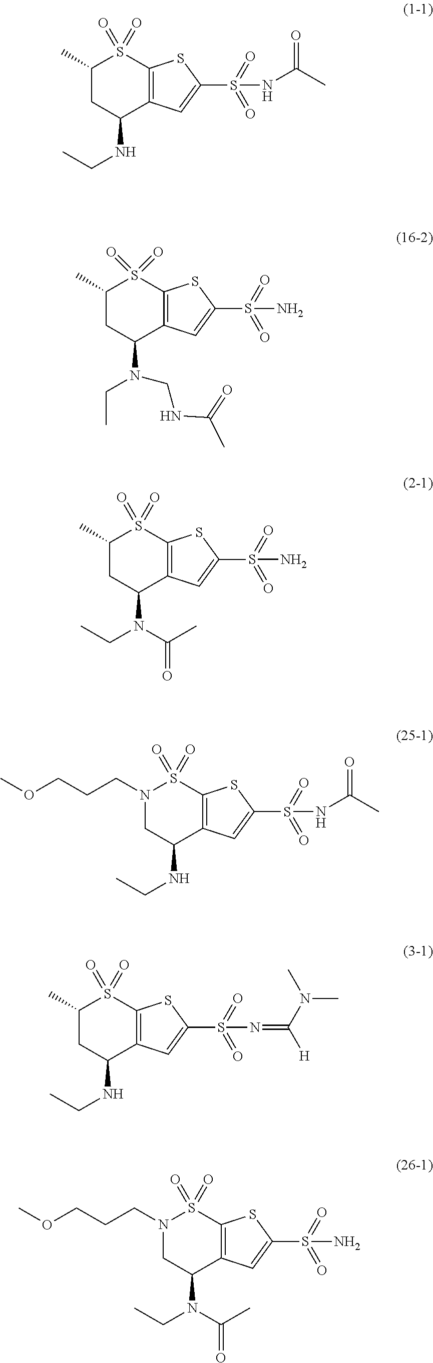

In another embodiment, Compound 1-1, Compound 2-1, Compound 3-1, Compound 16-2, Compound 25-1, or Compound 26-1 or a pharmaceutically acceptable salt thereof is provided to the patient by administration to the eye via intravitreal, intrastromal, intracameral, sub-tenon, sub-retinal, retro-bulbar, peribulbar, suprachoroidal, choroidal, subchoroidal, conjunctival, episcleral, posterior juxtascleral, circumcorneal, or tear duct injection in combination with one or more pharmaceutically acceptable carriers. In certain aspects, Compound 1-1, Compound 2-1, Compound 3-1, Compound 16-2, Compound 25-1, or Compound 26-1 or a pharmaceutically acceptable salt thereof are administered in a site that is not near the trabecular meshwork. In certain aspects, Compound 1-1, Compound 2-1, Compound 3-1, Compound 16-2, Compound 25-1, or Compound 26-2 or a pharmaceutically acceptable salt thereof is administered via subconjunctival injection.

Compounds of Formula I are single agent prodrugs of Sunitinib or a pharmaceutically acceptable salt thereof.

Compounds of Formula II, Formula IV, Formula VI, and Formula VIII are single agent prodrugs of Dorzolamide or a pharmaceutically acceptable salt thereof.

Compounds of Formula III, Formula V, Formula VII, and Formula IX are single agent prodrugs of Brinzolamide or a pharmaceutically acceptable salt thereof.

Compounds of Formula XII and Formula XIV are prodrug conjugates of Dorzolamide and Timolol, Sunitinib, or Bumetanide allowing release of both compounds in the eye. In one embodiment both compounds are released concurrently.

Compounds of Formula XI and Formula XIII are prodrug conjugates of Brinzolamide and Timolol, Sunitinib, or Bumetanide allowing release of both compounds in the eye. In one embodiment both compounds are released concurrently.

This invention also includes microparticles for ocular delivery that include an agent selected from Compound 1-1, Compound 2-1, Compound 3-1, Compound 16-2, Compound 25-1, or Compound 26-1 wherein the microparticles release the agent for at least about 1 month, 2 months, 3 months, 4 months, 5 months, or 6 months. In one embodiment, the microparticles have a diameter greater than 10 μM and include a core comprising one or more biodegradable polymers and a therapeutic agent selected from Compound 1-1, Compound 2-1, Compound 3-1, Compound 16-2, Compound 25-1, or Compound 26-1. In non-limiting embodiments, the microparticles have a diameter from about 10 μm to 60 μm, from about 20 μm to about 40 μm, or from about 25 μM to about 35 μM. In one non-limiting embodiment, the microparticle comprises Compound 1-1, Compound 2-1, Compound 3-1, Compound 16-2, Compound 25-1, or Compound 26-1 encapsulated in a blend of one or more hydrophobic polymers and an amphiphilic polymer. As discussed above, the one or more hydrophobic polymers and amphiphilic polymer are, for example (i) a PLGA polymer or PLA polymer as described herein and (ii) a PLGA-PEG or PLA-PEG copolymer; (i) a PLGA polymer; (ii) a PLA polymer; and, (iii) a copolymer of PLGA-PEG or PLA-PEG; or (i) a PLA polymer; (ii) a PLGA polymer; (iii) a PLGA polymer that has a different ratio of lactide and glycolide monomers than the PLGA in (ii); and, (iv) a PLGA-PEG or PLA-PEG copolymer.

The invention includes the use of a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or a pharmaceutically acceptable salt or composition thereof for the treatment of an ocular disorder wherein the compound is administered via intravitreal, intrastromal, intracameral, sub-tenon, sub-retinal, retro-bulbar, peribulbar, suprachoroidal, choroidal, subchoroidal, conjunctival, episcleral, posterior juxtascleral, circumcorneal, or tear duct injection, or through a mucus, mucin, or a mucosal barrier, in an immediate or controlled release fashion.

In one embodiment, a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or a pharmaceutically acceptable salt or composition thereof is administered via subconjunctival injection.

In one embodiment, a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or a pharmaceutically acceptable salt or composition thereof is administered in a dosage form that contains from about 1 μg to 10 mg, from about 1 μg to 1 mg, from about 1 μg to 100 μg, from about 1 μg to 50 μg, from about 1 μg to 10 μg, or from about 1 μg to 5 μg.

Another embodiment is provided that includes the administration of an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or a pharmaceutically acceptable salt or composition thereof, optionally in a pharmaceutically acceptable carrier, including a polymeric carrier, to a host to treat an ocular or other disorder that can benefit from topical or local delivery. The therapy can be delivery to the anterior or posterior chamber of the eye. In specific aspects, the active compound is administered to treat a disorder of the cornea, conjunctiva, aqueous humor, iris, ciliary body, lens sclera, choroid, retinal pigment epithelium, neural retina, optic nerve or vitreous humor.

In an alternative embodiment, any of the compounds or pharmaceutically acceptable salts thereof can be administered systemically, topically, parentally, intravenously, subcutaneously, intramuscularly, transdermally, buccally, or sublingually in an effective amount.

In any of the Formulas described herein (Formula I, Formula II, Formula III, Formula IV, Formula V, Formula VI, Formula VII, Formula VIII, Formula IX, Formula X, Formula XI, Formula XII, Formula XIII, Formula XIV, or Formula XV) if the stereochemistry of a chiral carbon is not specifically designated in the Formula it is intended that the carbon can be used as an R enantiomer, an S enantiomer, or a mixture of enantiomers including a racemic mixture. Likewise, compounds presented which are or are analogs of commercial products are provided in their approved stereochemistry for regulatory use, unless stated otherwise.

In addition, moieties that have repetitive units of the same or varying monomers, for example including, but not limited to an oligomer of polylactic acid, polylactide-coglycolide, or polypropylene oxide, that have a chiral carbon can be used with the chiral carbons all having the same stereochemistry, random stereochemistry (by either monomer or oligomer), racemic (by either monomer or oligomer) or ordered but different stereochemistry such as a block of S enantiomer units followed by a block of R enantiomer units in each oligomeric unit. In some embodiments lactic acid is used in its naturally occurring S enantiomeric form.

In certain embodiments, the conjugated active drug is delivered in a biodegradable microparticle or nanoparticle that has at least approximately 5, 7.5, 10, 12.5, 15, 20, 25 or 30% or more by weight conjugated active drug. In some embodiments, the biodegradable microparticle degrades over a period of time and in any event provides controlled delivery that lasts at least approximately 2 months, 3 months, 4 months, 5 months or 6 months or more. In some embodiments, the loaded microparticles are administered via subconjunctival or subchoroidal injection.

In certain embodiments, the conjugated active drug is delivered as the pharmaceutically acceptable salt form. Salt forms of a compound will exhibit distinctive solution and solid-state properties compared to their respective free base or free acid form, and for this reason pharmaceutical salts are used in drug formulations to improve aqueous solubility, chemical stability, and physical stability issues. Lipophilic salt forms of compounds, which have enhanced solubility in lipidic vehicles relative to the free acid or free base forms of compounds, are often advantageous in terms of pharmacological properties due in part to their low melting points. Lipophilic salt forms of compounds are used to increase aqueous solubility for oral and parenteral drug delivery, enhance permeation across hydrophobic barriers, and enhance drug loading in lipid-based formulations.

In all of the polymer moieties described in this specification, where the structures are depicted as block copolymers (for example, blocks of “x” followed by blocks of “y”) it is intended that the polymer can alternately be a random or alternating copolymer (for example, “x” and “y”, are either randomly distributed or alternate). Unless stereochemistry is specifically indicated, each individual moiety of each oligomer that has a chiral center can be presented at the chiral carbon in (R) or (S) configuration or a mixture there of, including a racemic mixture.

In most of the Formulas presented herein, the prodrugs are depicted as one or several active moieties covalently bound to or through a described prodrug moiety(ies) with a defined variable range of each of the active moiety and the prodrug moiety through the use of descriptors x, y, m or n. As indicated below, these descriptors can independently have numerical ranges provided below, and in most embodiments, are typically within a smaller range, also as provided below. Each variable is independent such that any of the integers of one variable can be used with any of the integers of the other variable, and each combination is considered separately and independently disclosed, and set out below like this only for space considerations.

For example, x and y can independently be any integer between 1 and 20 (1, 2, 3, 4, 5, 6, 7, 8, 9, 10, 11, 12, 13, 14, 15, 16, 17, 18, 19, or 20). In certain embodiments, x or y can independently be 1, 2, 3, 4, 5, 6, 7, 8, 9, or 10, 11, or 12 and in certain aspects, 1, 2, 3, 4, 5, or 6. In certain embodiments, x is 1, 2, 3, 4, 5, 6, 7, or 8. In certain embodiments, y is 1, 2, 3, 4, 5, 6, 7, or 8. In certain embodiments, x is 1, 2, 3, 4, 5, or 6. In certain embodiments, y is 1, 2, 3, 4, 5, or 6. In certain embodiments, y is 1, 2, or 3 and x is 1, 2, 3, 4, 5, or 6. In certain embodiments, x is 1, 2, or 3 and y is 1, 2, 3, 4, 5, or 6. In certain embodiments, x is an integer selected from 1, 2, 3, and 4 and y is 1. In certain embodiments, xis an integer selected from 1, 2, 3, and 4 and y is 2. In certain embodiments, x is in integer selected from 1, 2, 3, and 4 and y is 3. x and y can independently be

Variables m and n can also be any integer between 1 and 20 (1, 2, 3, 4, 5, 6, 7, 8, 9, 10, 11, 12, 13, 14, 15, 16, 17, 18, 19, or 20). In certain embodiments, m or n can independently be 1, 2, 3, 4, 5, 6, 7, 8, 9, or 10, 11, or 12 and in certain aspects, 1, 2, 3, 4, 5, or 6. In certain embodiments, m is 1, 2, 3, 4, 5, 6, 7, or 8. In certain embodiments, n is 1, 2, 3, 4, 5, 6, 7, or 8. In certain embodiments, m is 1, 2, 3, 4, 5, or 6. In certain embodiments, n is 1, 2, 3, 4, 5, or 6. In certain embodiments, n is 1, 2, or 3 and m is 1, 2, 3, 4, 5, or 6. In certain embodiments, m is 1, 2, or 3 and n is 1, 2, 3, 4, 5, or 6. In certain embodiments, m is an integer selected from 1, 2, 3, and 4 and n is 1. In certain embodiments, m is an integer selected from 1, 2, 3, and 4 and n is 2. In certain embodiments, m is in integer selected from 1, 2, 3, and 4 and n is 3.

Where x or y is used in connection with the monomeric residue in an oligomer, including for example but not limited to:

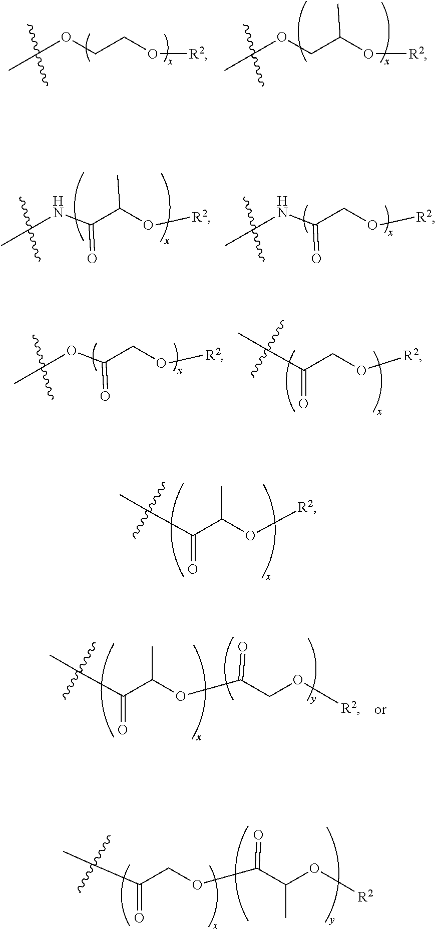

then x or y is in some embodiments independently 1, 2, 3, 4, 5, 6, 7 or 8, and even for example, 2, 4 or 6 residues.

Where m or n is used in connection with the monomeric residue in an oligomer, including for example but not limited to:

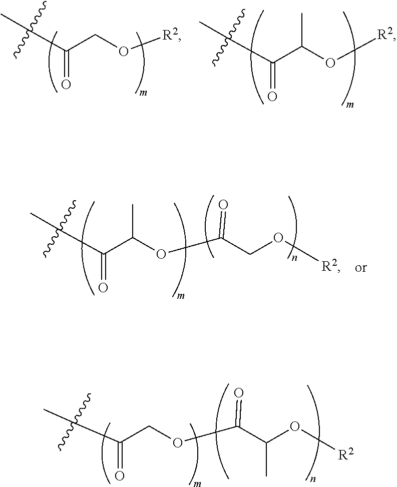

then m or n is in some embodiments independently 1, 2, 3, 4, 5, 6, 7 or 8, and even for example, 2, 4 or 6 residues.

This disclose provides a compound of Formula (I):

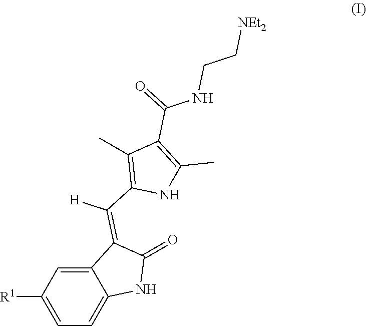

or a pharmaceutically acceptable salt thereof,

wherein

R¹ is selected from

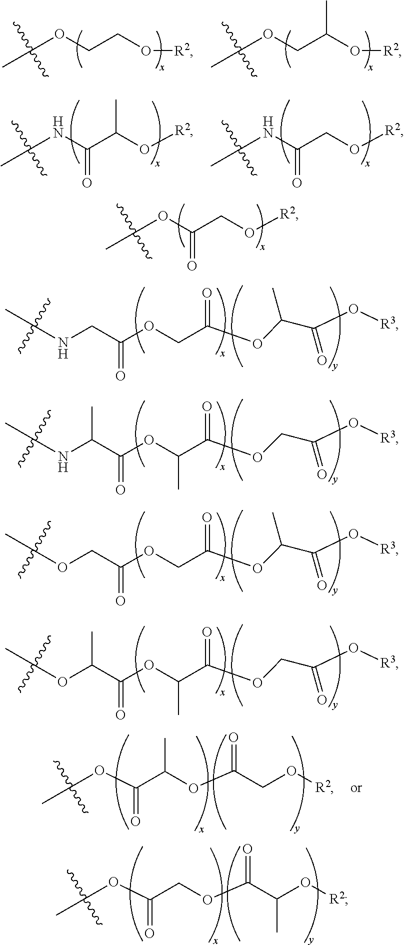

R² is selected from hydrogen, —CH₂COOH, —C(O)R⁴, alkenyl, alkynyl, cycloalkyl, cycloalkylalkyl, heterocyclyl, heterocycloalkyl, aryl, arylalkyl, heteroaryl, and heteroarylalkyl;

R³ is selected from hydrogen, alkenyl, alkynyl, cycloalkyl, cycloalkylalkyl, heterocyclyl, heterocycloalkyl, aryl, arylalkyl, heteroaryl, and heteroarylalkyl;

R⁴ is selected from hydrogen, alkyl, cycloalkyl, cycloalkylalkyl, heterocycle, heterocycloalkyl, aryl, arylalkyl, heteroaryl, and heteroarylalkyl wherein each group can be optionally substituted with another desired substituent group which results in a pharmaceutically acceptable compound and is sufficiently stable under the conditions of use, for example selected from R⁵;

R⁵ is selected from: halogen, hydroxyl, cyano, mercapto, amino, alkyl, alkoxy, alkenyl, alkynyl, cycloalkyl, cycloalkylalkyl, heterocyclyl, heterocycloalkyl, aryl, arylalkyl, heteroaryl, heteroarylalkyl, aryloxy, —S(O)₂alkyl, —S(O)alkyl, —P(O)(Oalkyl)₂, B(OH)₂, —Si(CH₃)₃, —COOH, —COOalkyl, and —CONH₂, each of which except halogen, cyano, and —Si(CH₃)₃ may be optionally substituted, for example with halogen, alkyl, aryl, heterocycle or heteroaryl if desired and if the resulting compound achieves the desired purpose, wherein the group cannot be substituted with itself, for example alkyl would not be substituted with alkyl; and

x and y are an integer independently selected from 1, 2, 3, 4, 5, 6, 7, 8, 9, 10, 11, 12, 13, 14, 15, 16, 17, 18, 19, and 20.

Non-limiting examples of R¹ include

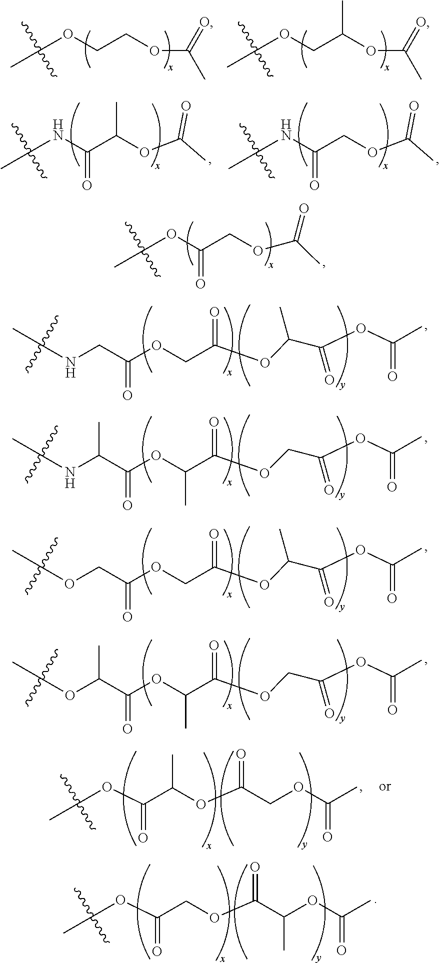

This disclosure also provides a compound of Formula (II) and Formula (III):

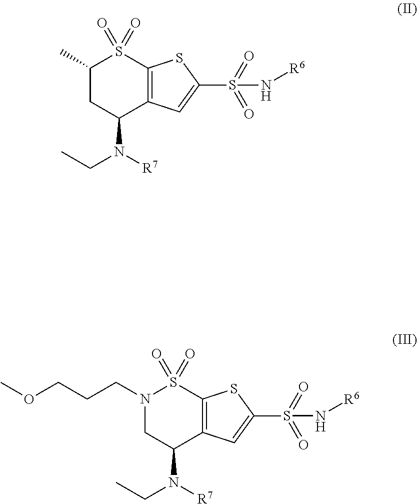

or a pharmaceutically acceptable salt thereof,

wherein

R⁶ is selected from

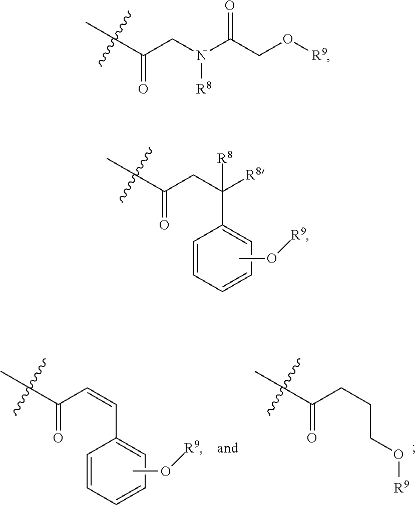

R⁷ is hydrogen or —C(O)R⁴;

R⁸ and R^{8′} are independently selected from hydrogen and C₁₋₆alkyl;

R⁹ is —C(O)R⁴, —C(O)CH₂OR⁴,

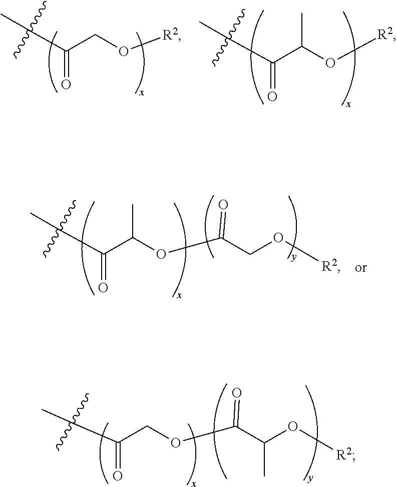

or in an alternative embodiment, R⁹ is

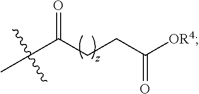

z is an integer independently selected from 0, 1, 2, 3, 4, 5, 6, 7, 8, 9, or 10; and

R², R⁴, x, and y are defined herein.

Non-limiting examples of R⁶ include

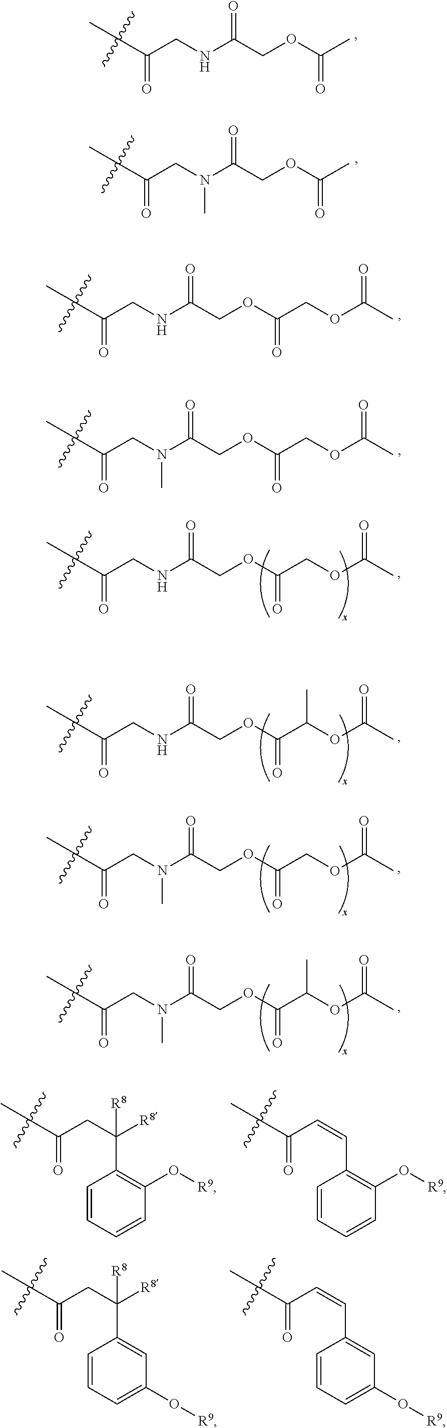

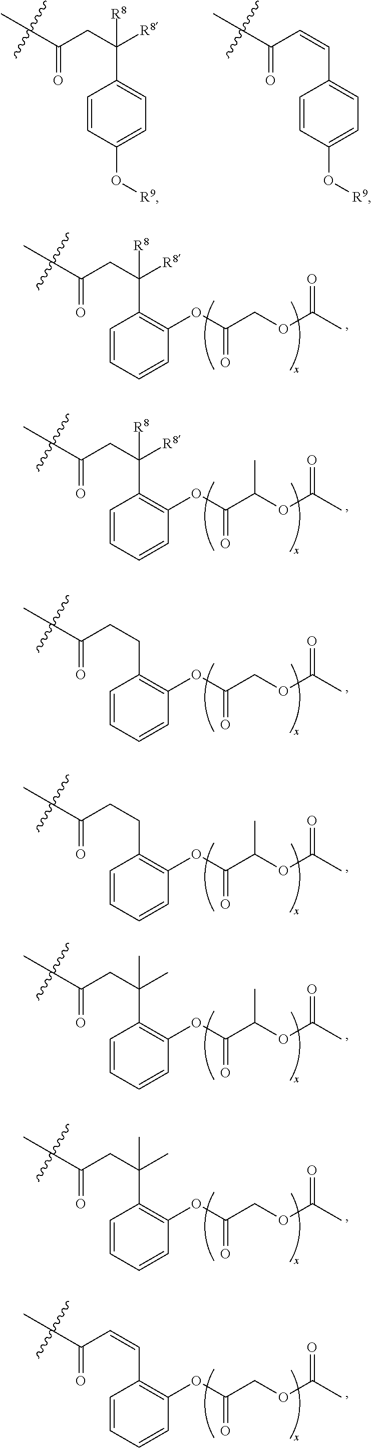

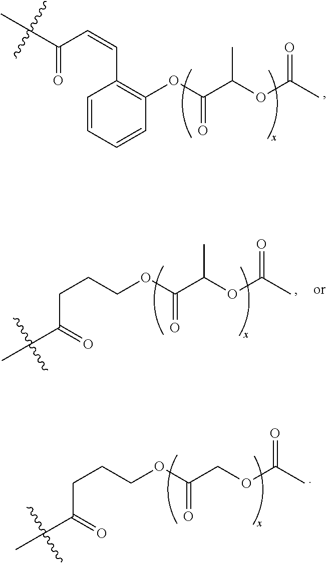

In one embodiment, R⁷ is hydrogen.

In one embodiment, R⁷ is —C(O)R⁴.

In one embodiment, R⁹ is —C(O)R⁴ and R⁴ is methyl.

In one embodiment, R⁷ is hydrogen and R⁶ is

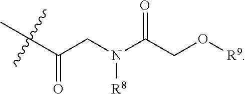

In one embodiment, R⁷ is hydrogen, R⁶ is

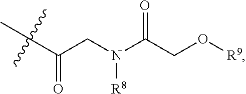

R⁹ is —C(O)R⁴, and R⁴ is methyl.

In one embodiment, R⁷ is hydrogen, R⁶ is

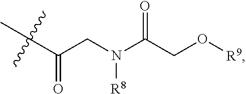

and R⁸ is methyl.

In one embodiment, R⁷ is hydrogen, R⁶ is

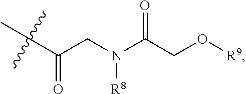

and R⁸ is hydrogen.

In one embodiment, R⁷ is hydrogen, and R⁶ is

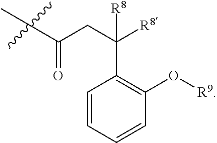

In one embodiment, R⁷ is hydrogen, R⁶ is

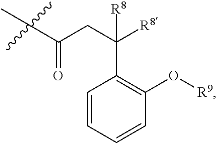

and R⁸ and R^{8′} are hydrogen.

In one embodiment, R⁷ is hydrogen, R⁶ is

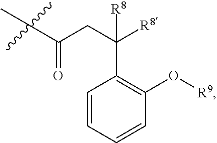

and R⁸ and R^{8′} are methyl.

In one embodiment, R⁷ is hydrogen and R⁶ is

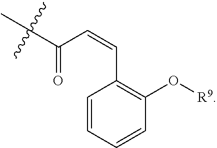

In one embodiment, R⁷ is hydrogen and R⁶ is

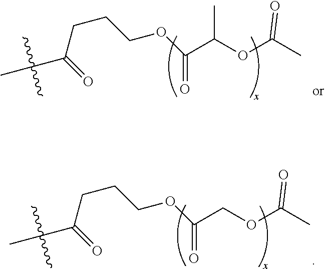

In an alternative embodiment, R⁹ is

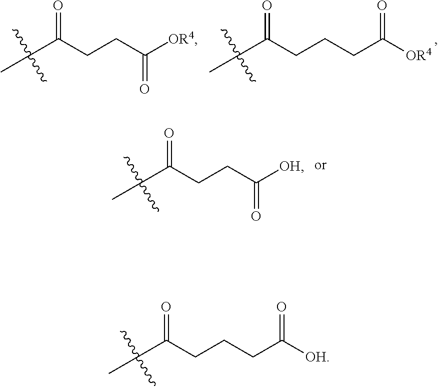

In an alternative embodiment, z is an integer selected from 0, 1, 2, 3, 4, 5, and 6. In an alternative embodiment, z is an integer selected from 1, 2, or 3.

This disclosure also provides a compound of Formula (IV) and Formula (V):

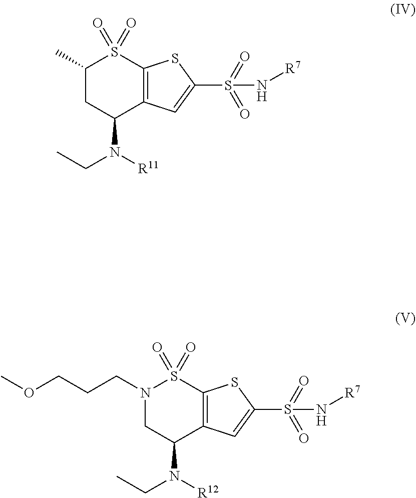

or a pharmaceutically acceptable salt thereof,

wherein

R⁷ is hydrogen or —C(O)R⁴;

R¹¹ is selected from

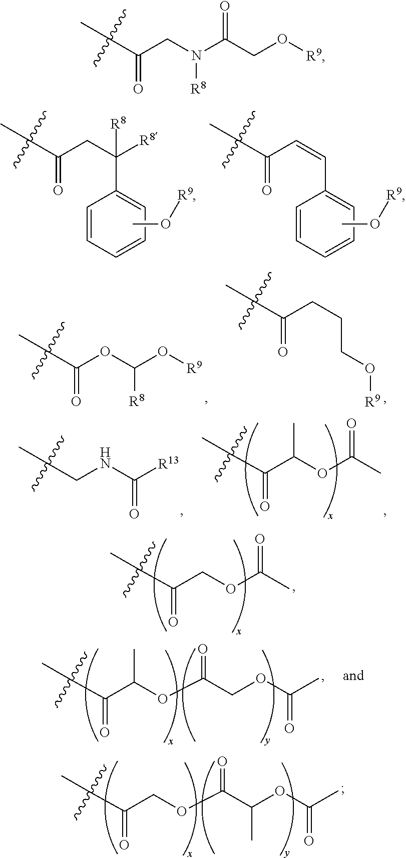

R¹² is selected from

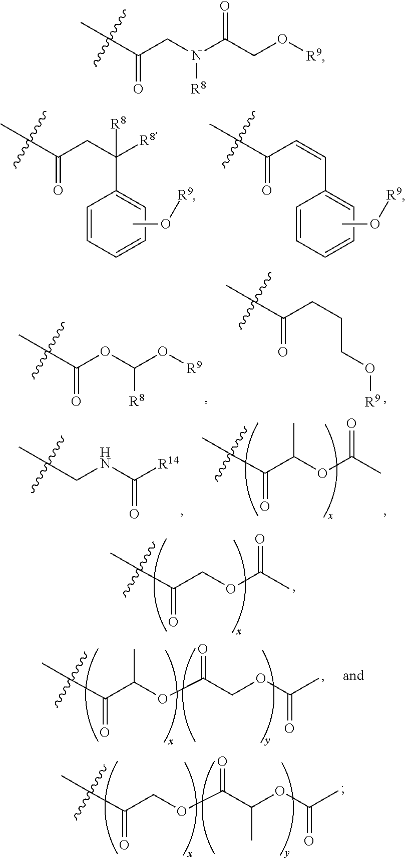

R¹³ is independently selected from C₄₋₆alkyl, C₃₋₇cycloalkyl, cycloalkylalkyl, heterocycle, heterocycloalkyl, aryl, arylalkyl, heteroaryl, heteroarylalkyl wherein each group can be optionally substituted with another desired substituent group which results in a pharmaceutically acceptable compound and is sufficiently stable under the conditions of use, for example selected from R⁵;

R¹⁴ is independently selected from C₁₋₆alkyl, C₃₋₇cycloalkyl, cycloalkylalkyl, heterocycle, heterocycloalkyl, aryl, arylalkyl, heteroaryl, heteroarylalkyl wherein each group can be optionally substituted with another desired substituent group which results in a pharmaceutically acceptable compound and is sufficiently stable under the conditions of use, for example selected from R⁵; and

R⁴, R⁵, R⁸, R^{8′}, R⁹, x and y are defined herein.

Non-limiting examples of R¹¹ or R¹² include

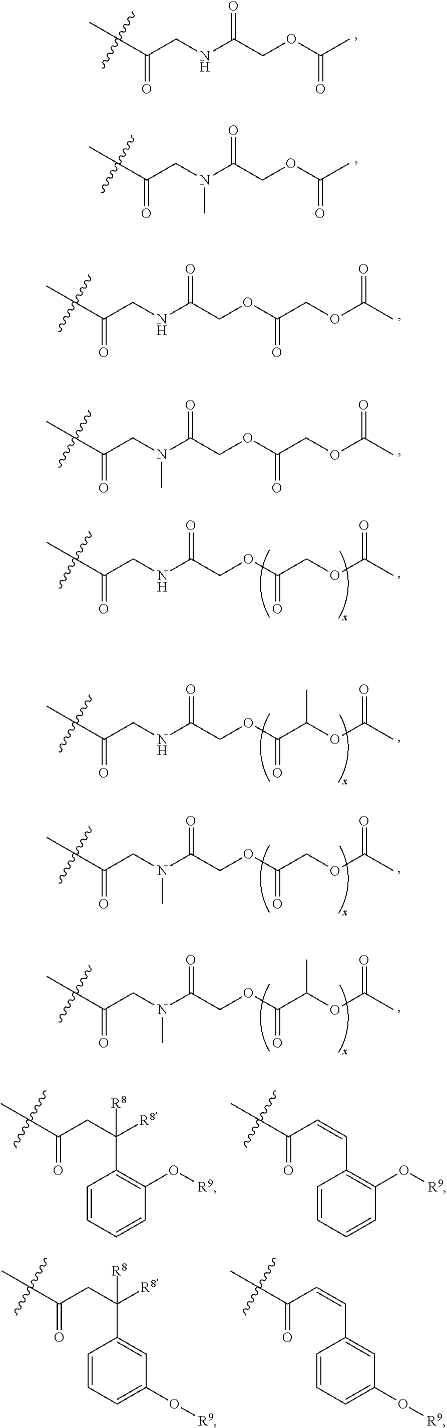

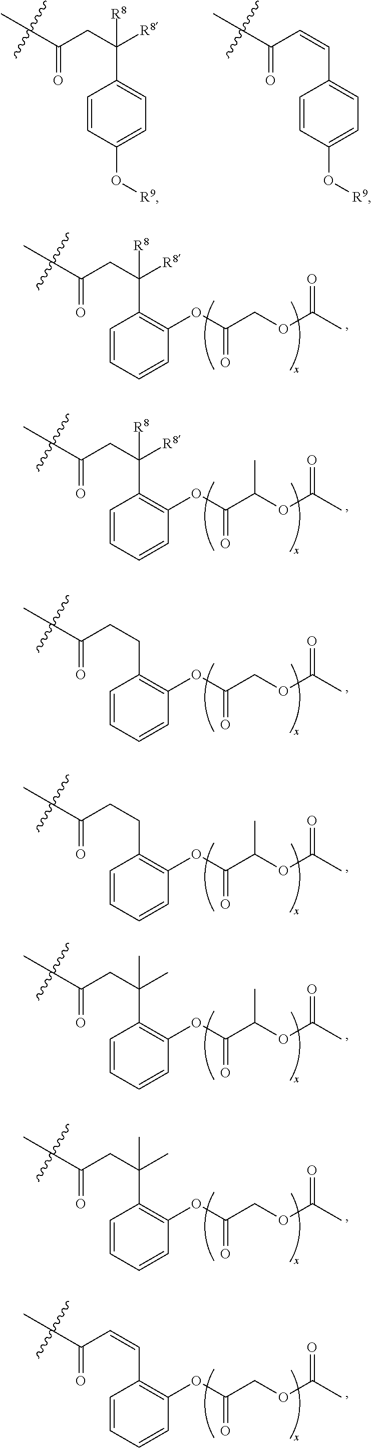

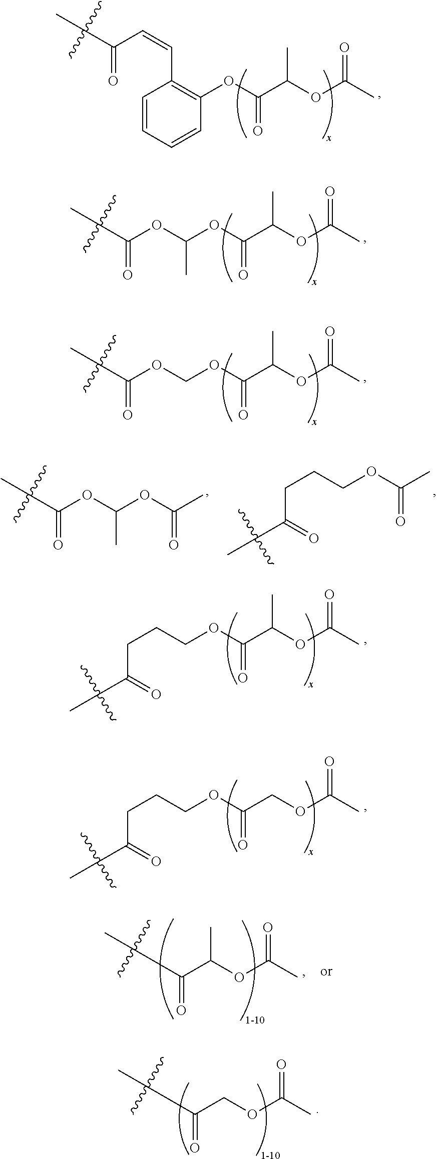

In one embodiment, R⁷ is hydrogen.

In one embodiment, R⁷ is —C(O)R⁴.

In one embodiment, R⁷ is hydrogen and R¹¹ is

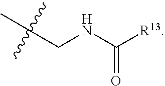

In one embodiment, R⁷ is hydrogen and R¹² is

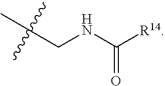

In one embodiment, R⁷ is hydrogen and R¹² is

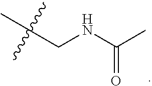 [SMILES: *CNC(C)=O]

In one embodiment, R⁷ is hydrogen and R¹¹ or R¹² is

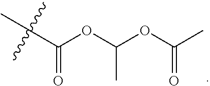 [SMILES: *C(=O)OC(C)OC(C)=O]

In one embodiment, R⁷ is hydrogen and R¹¹ or R¹² is

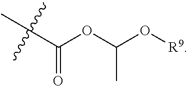

In one embodiment, R⁷ is hydrogen and R¹¹ or R¹² is

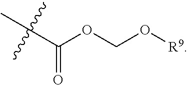

In one embodiment, R⁷ is hydrogen and R¹¹ or R¹² is

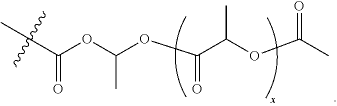

In one embodiment, R⁷ is hydrogen and R¹¹ or R¹² is

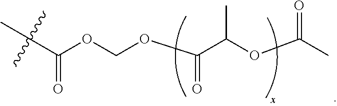

In one embodiment, R⁹ is —C(O)R⁴ and R⁴ is methyl.

In one embodiment, R⁷ is hydrogen and R¹¹ or R¹² is

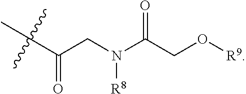

In one embodiment, R⁷ is hydrogen, R¹¹ or R¹² is

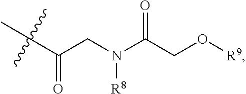

R⁹ is —C(O)R⁴, and R⁴ is methyl.

In one embodiment, R⁷ is hydrogen, R¹¹ or R¹² is

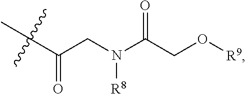

and R⁸ is methyl.

In one embodiment, R⁷ is hydrogen, R¹¹ or R¹² is

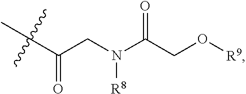

and R⁸ is hydrogen.

In one embodiment, R⁷ is hydrogen, and R¹¹ or R¹² is

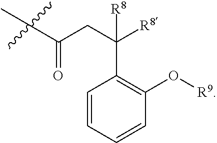

In one embodiment, R⁷ is hydrogen, R¹¹ or R¹² is

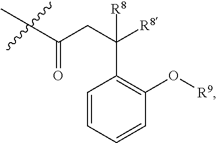

and R⁸ and R^{8′} are hydrogen.

In one embodiment, R⁷ is hydrogen, R¹¹ or R¹² is

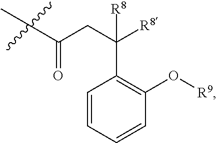

and R⁸ and R^{8′} are methyl.

In one embodiment, R⁷ is hydrogen, and R¹¹ or R¹² is

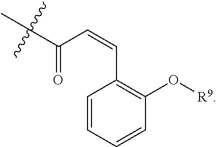

In one embodiment, R⁷ is hydrogen and R¹¹ is

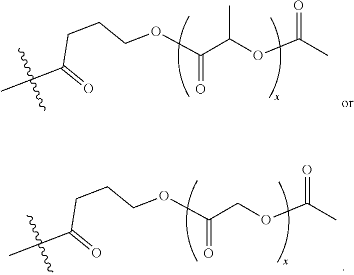

In an alternative embodiment, R¹¹ is selected from

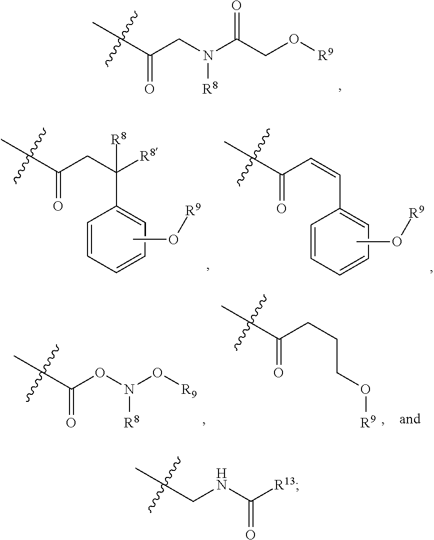

and

R¹² is selected from

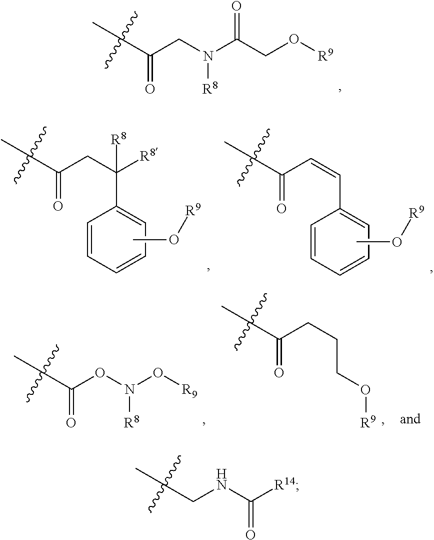

In an alternative embodiment, R⁷ is hydrogen and R¹¹ or R¹² is

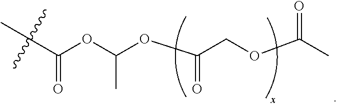

In an alternative embodiment, R⁷ is hydrogen and R¹¹ or R¹² is

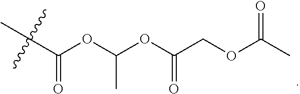 [SMILES: *C(=O)OC(C)OC(=O)COC(C)=O]

In an alternative embodiment, R⁷ is hydrogen and R¹¹ or R¹² is

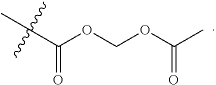 [SMILES: *C(=O)OCOC(C)=O]

In an alternative embodiment, R⁷ is hydrogen and R¹¹ or R¹² is

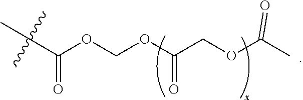

In an alternative embodiment, R⁷ is hydrogen and R¹¹ or R¹² is

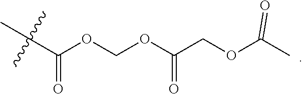 [SMILES: *C(=O)OCOC(=O)COC(C)=O]

In an alternative embodiment, R⁷ is hydrogen, R¹¹ or R¹² is

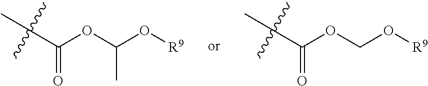

and R⁹ is —C(O)R⁴.

In a further embodiment, R⁴ is alkyl wherein alkyl is C₁-C₂₀, C₁-C₁₇, C₁-C₁₅, C₁-C₁₃, C₁-C₁₁, C₁-C₉, C₁-C₇, C₁-C₅, or C₁-C₃.

In a further embodiment, R⁴ is aryl wherein aryl is phenyl or benzyl.

This disclosure also provides a compound of Formula (VI) and Formula (VII):

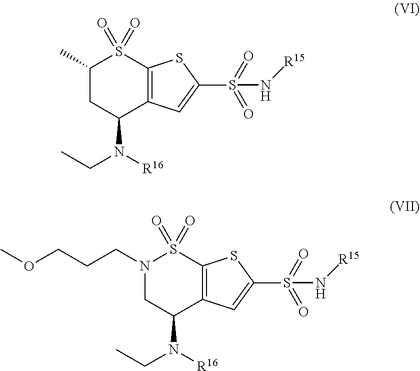

or a pharmaceutically acceptable salt thereof,

wherein

R¹⁵ is selected from —C(O)R⁴,

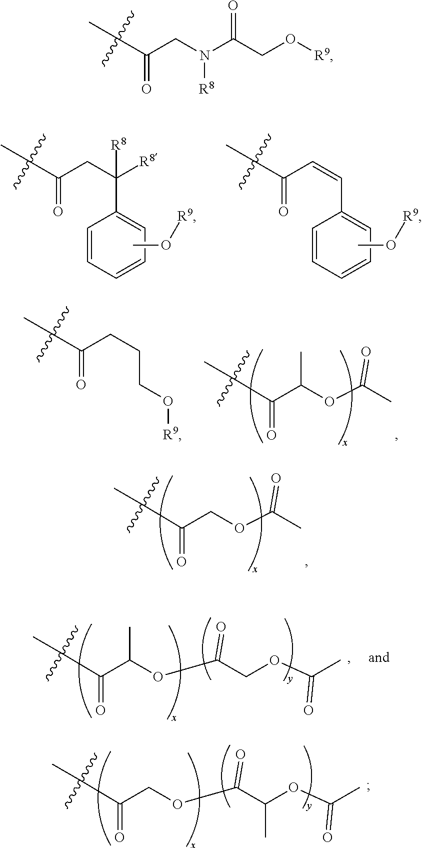

R¹⁶ is selected from

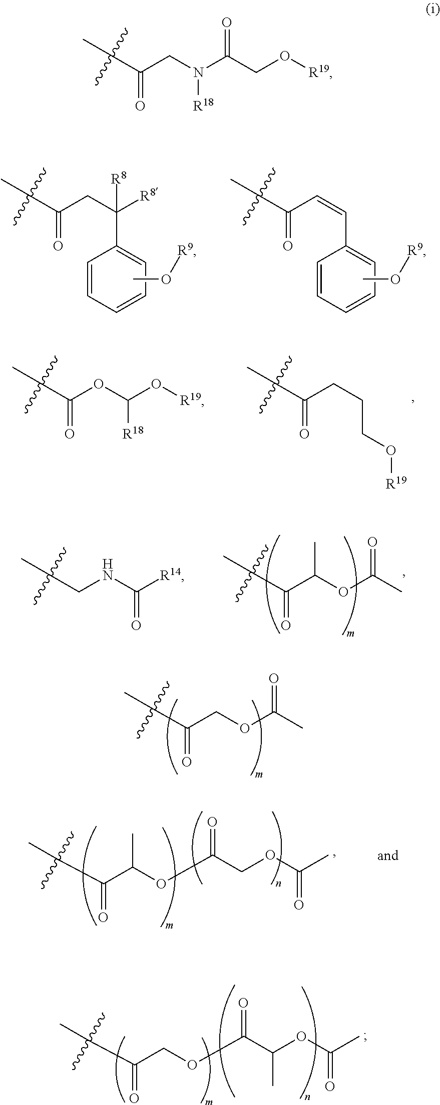

and
(ii) —C(O)R⁴ when R¹⁵ is —C(O)R⁴,

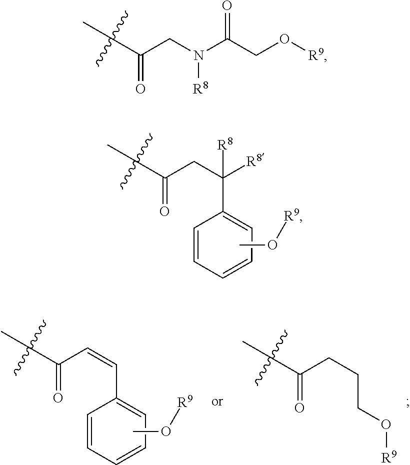

R¹⁸ and R^{18′} are independently selected from hydrogen and C₁₋₆alkyl; and

R¹⁹ is —C(O)R⁴, C(O)CH₂OR⁴,

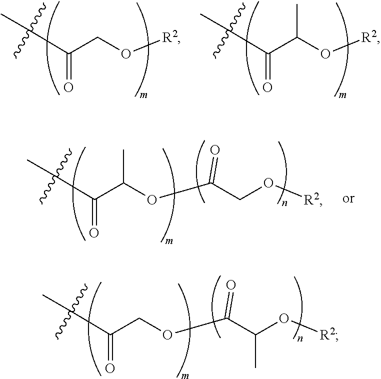

m and n are an integer independently selected from 1, 2, 3, 4, 5, 6, 7, 8, 9, 10, 11, 12, 13, 14, 15, 16, 17, 18, 19, and 20;

R², R⁴, R⁸, R^{8′}, R⁹, R¹², and R¹⁴ are defined herein.

In one embodiment, R¹⁵ and R¹⁶ are —C(O)R⁴ wherein R⁴ is methyl.

In one embodiment, R¹⁵ is

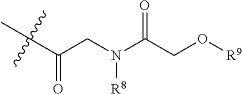

### and R¹⁶ is

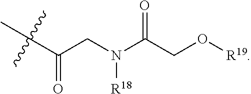

In one embodiment, R¹⁵ is

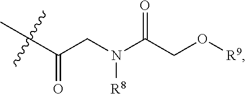

### R¹⁶ is

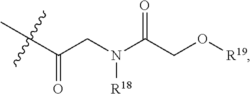

R⁸ is hydrogen, and R¹⁸ is hydrogen.

In one embodiment, R¹⁵ is

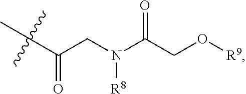

### R¹⁶ is

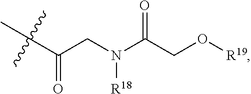

R⁸ is methyl, and R¹⁸ is methyl.

In one embodiment, R¹⁵ is

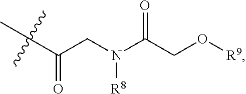

### R¹⁶ is

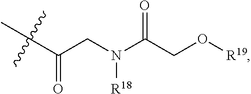

R⁹ is —C(O)R⁴, R¹⁹ is —C(O)R⁴, and R⁴ is methyl.

In one embodiment, R¹⁵ is

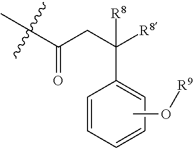

### and R¹⁶ is

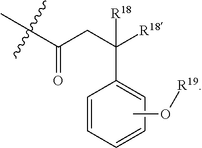

In one embodiment, R¹⁵ is

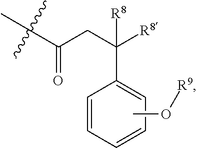

### R¹⁶ is

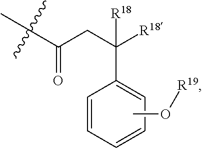

and R⁸, R^{8′}, R¹⁸, and R^{18′} are methyl.

In one embodiment, R¹⁵ is

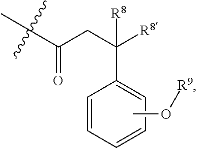

### R¹⁶ is

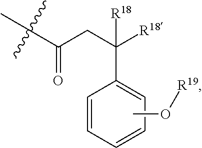

and R⁸, R^{8′}, R¹⁸, and R^{18′} are hydrogen.

In one embodiment, R¹⁵ is

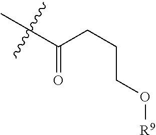

### and R¹⁶ is

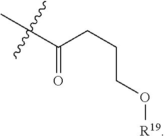

In one embodiment, R¹⁵ is

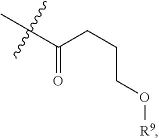

### R¹⁶ is

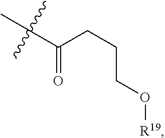

R⁹ is —C(O)R⁴, R¹⁹ is —C(O)R⁴, and R⁴ is methyl.

In one embodiment, R¹⁵ is

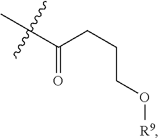

### R¹⁶ is

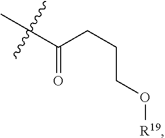

### R⁹ is

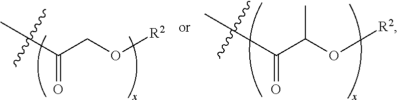

### R¹⁹ is

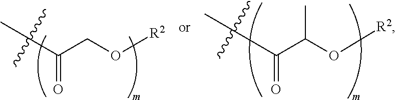

R² is —C(O)R⁴, and R⁴ is methyl.

In an alternative embodiment, R¹⁵ is selected from —C(O)R⁴,

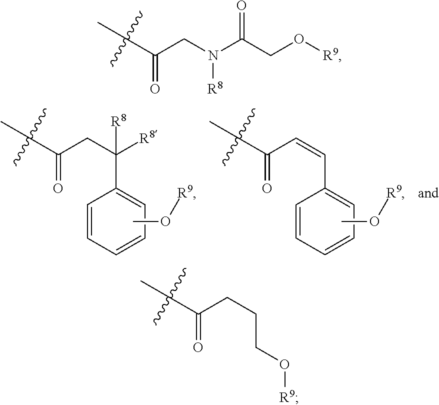

and

R¹⁶ is selected from

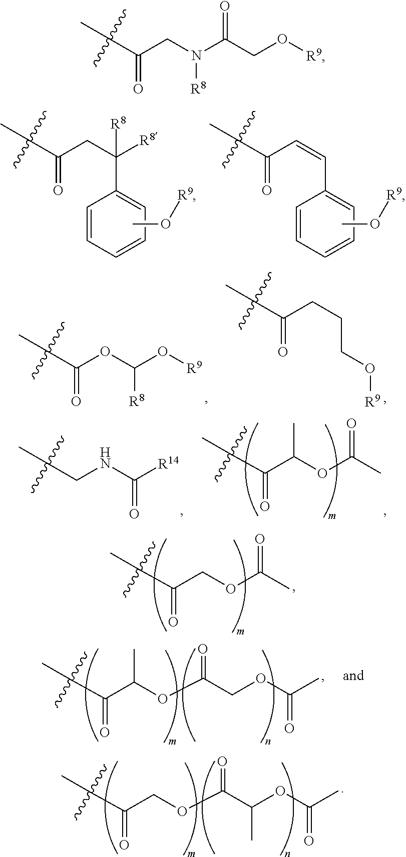

In an alternative embodiment, R¹⁵ is selected from —C(O)R⁴,

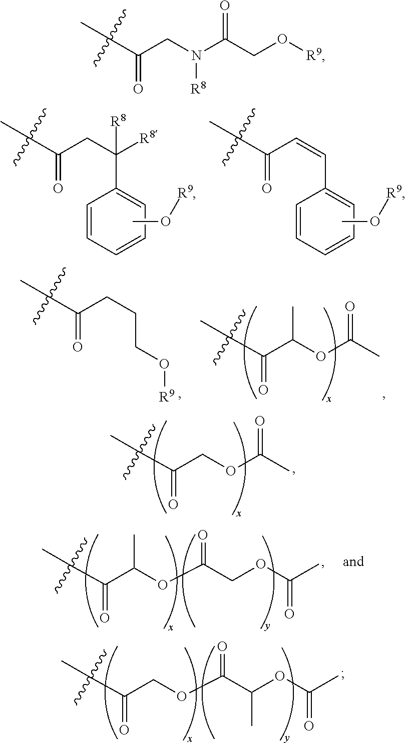

and

R¹⁶ is selected from

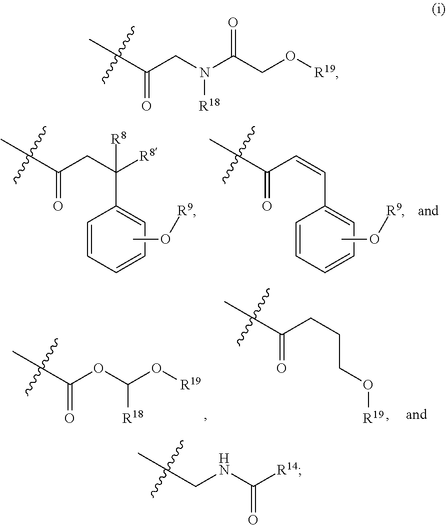

and
(ii) —C(O)R⁴ when R¹⁵ is —C(O)R⁴,

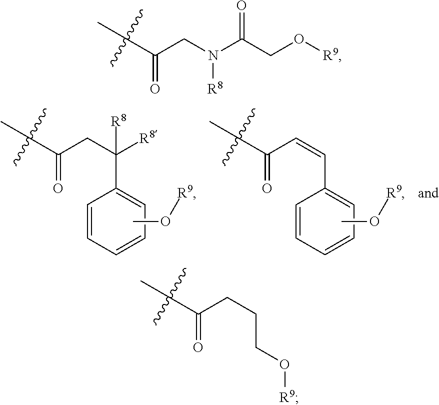

Non-limiting examples of R¹⁵ include

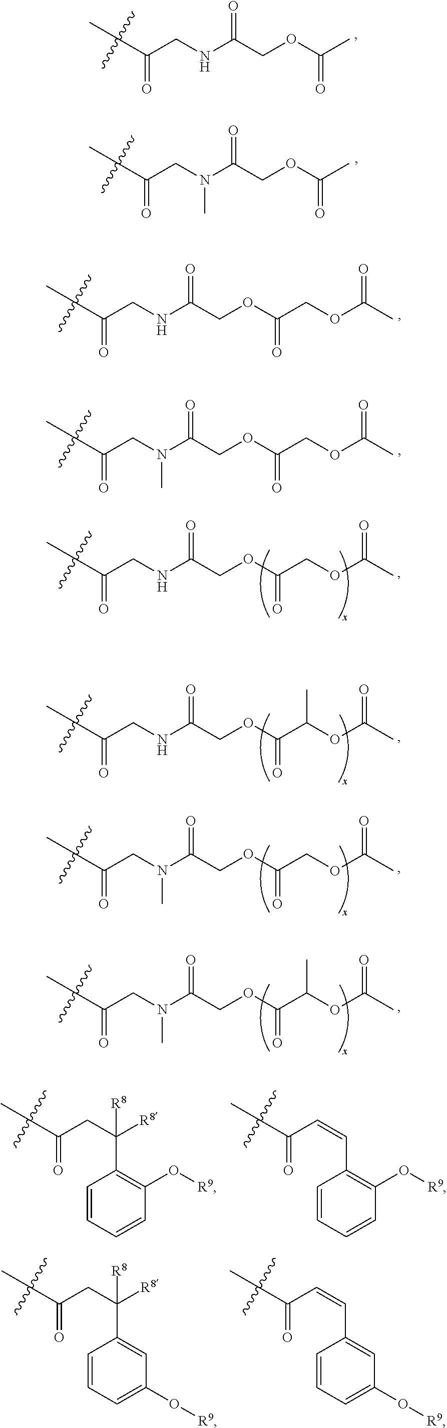

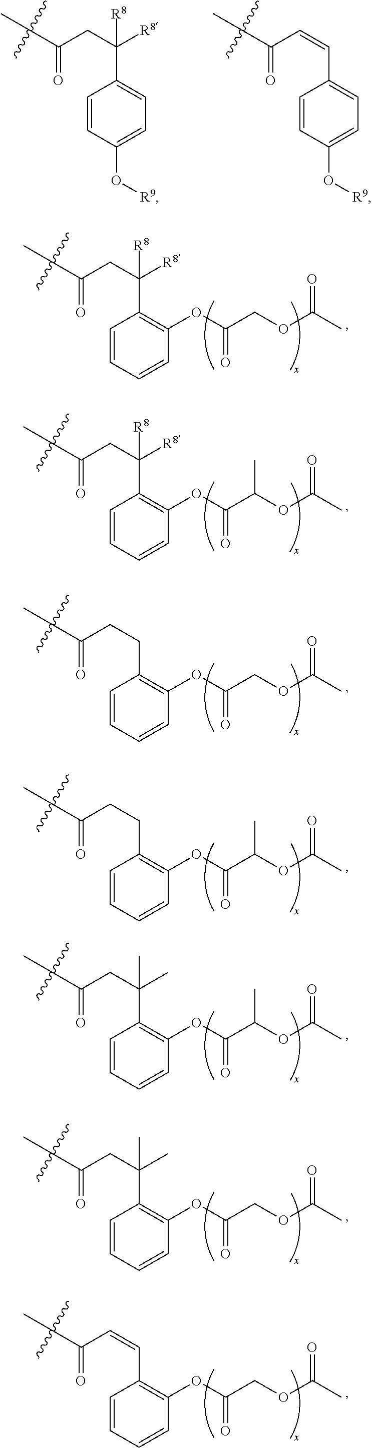

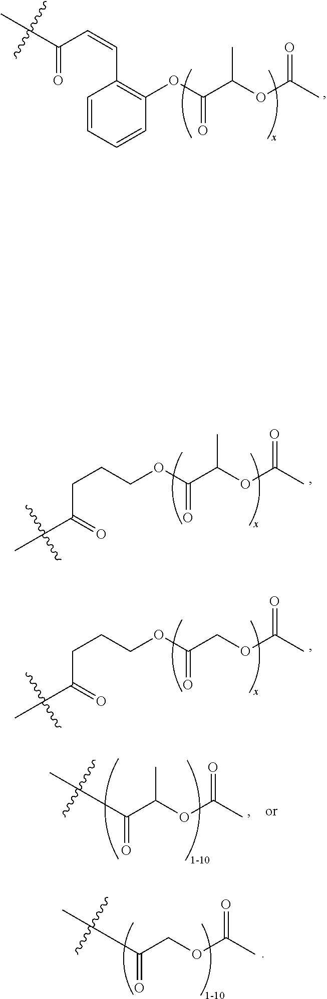

Non-limiting examples of R¹⁶ include

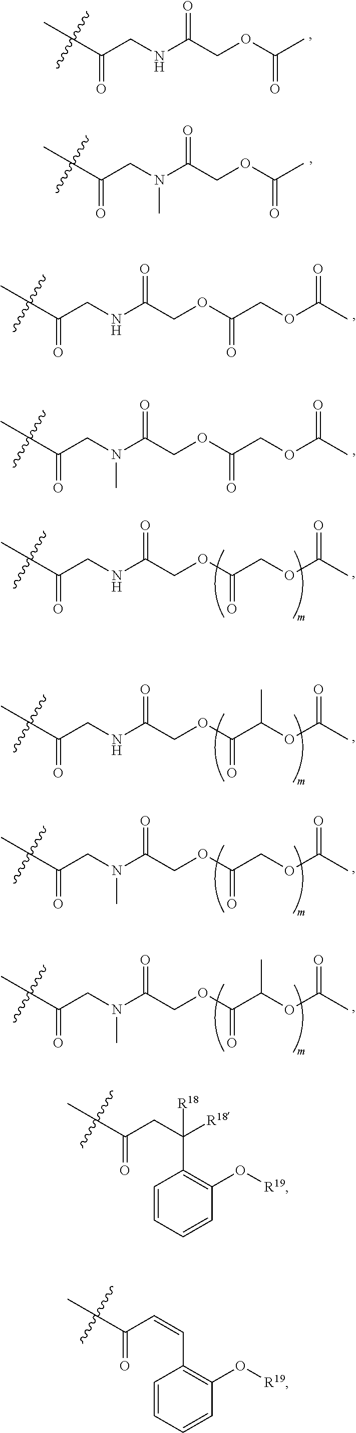

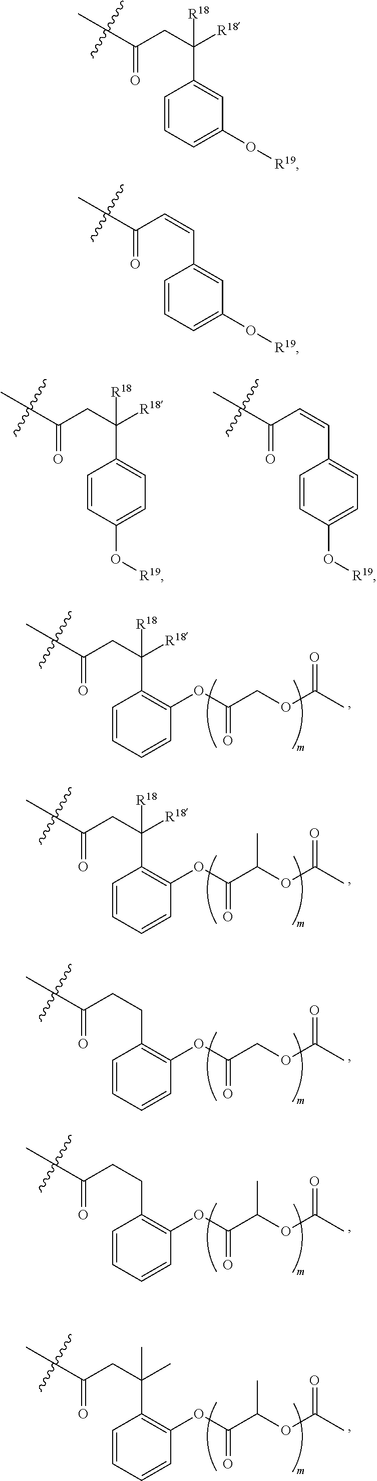

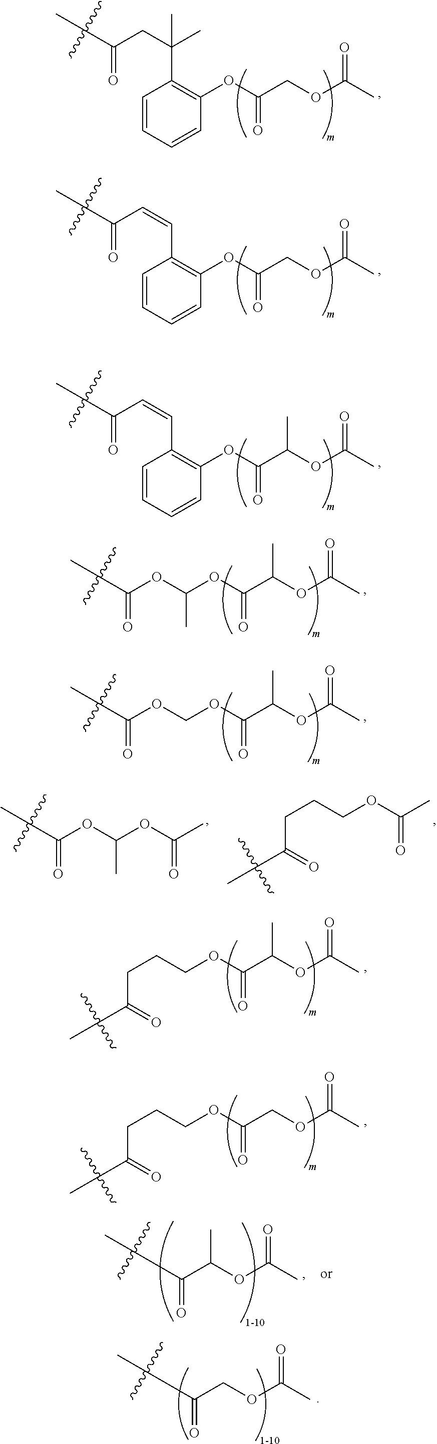

This disclosure also provides a compound of Formula (VIII), Formula (IX), Formula (X), and Formula (XI):

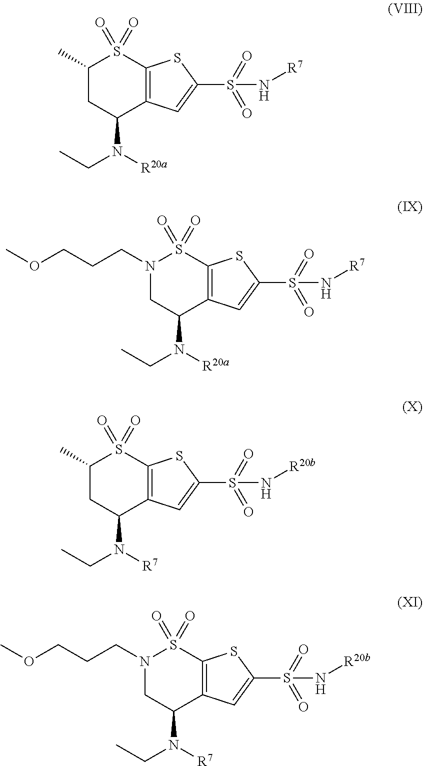

or a pharmaceutically acceptable salt thereof,

wherein

R⁷ is hydrogen or —C(O)R⁴;

R^{20a} is selected from

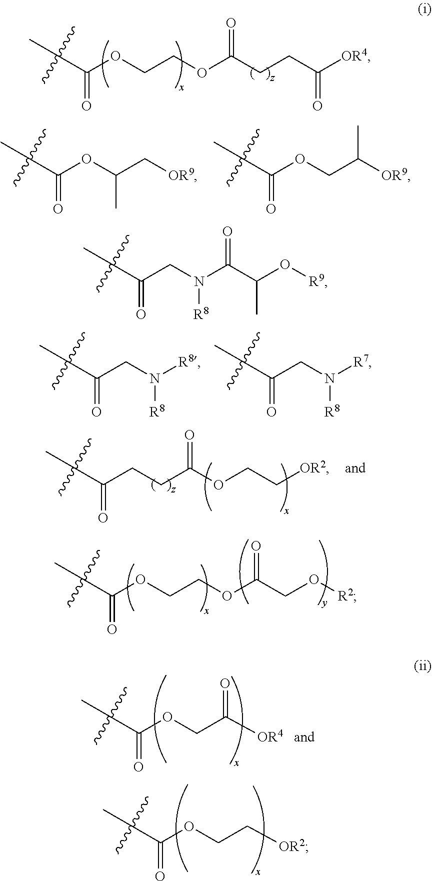

R^{20b} is selected from

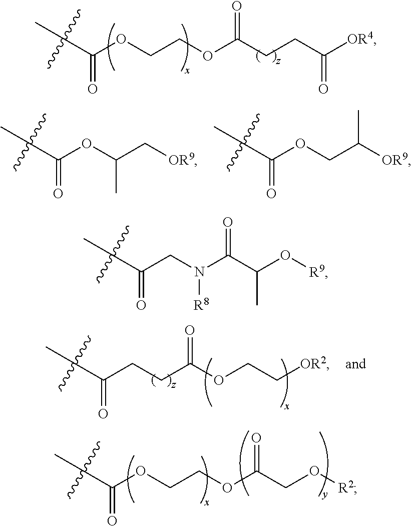

wherein R⁹ is not —C(O)R⁴ when R^{20b} is

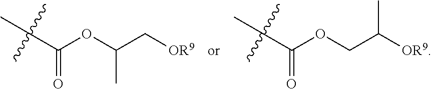

R², R⁴, R⁷, R⁸, R^{8′}, R⁹, x, y, and z are defined herein.

Non-limiting examples of R^{20a} include

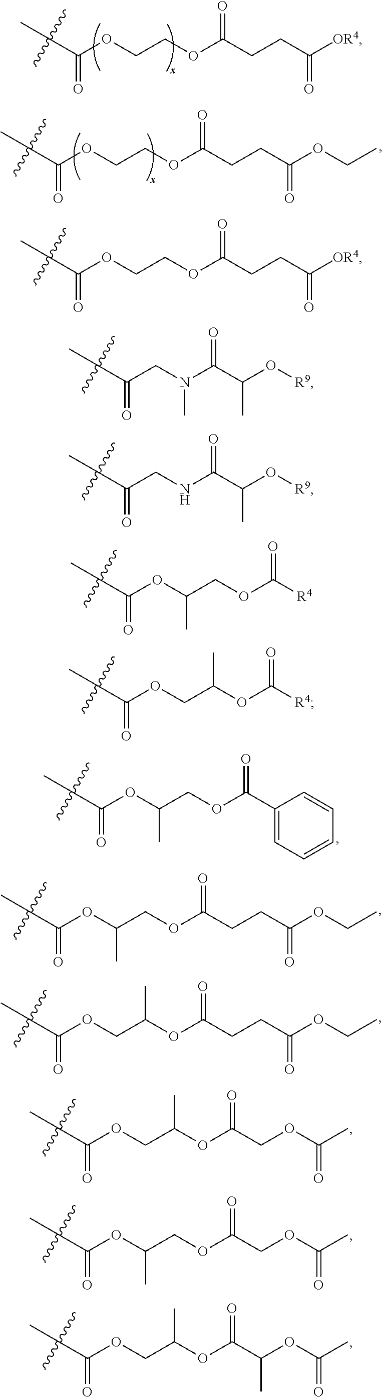

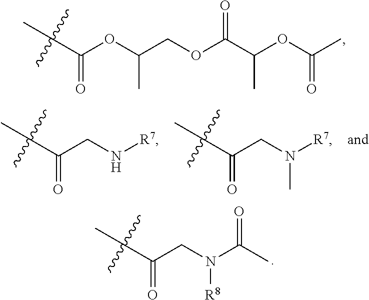

In one embodiment, R²⁰ is

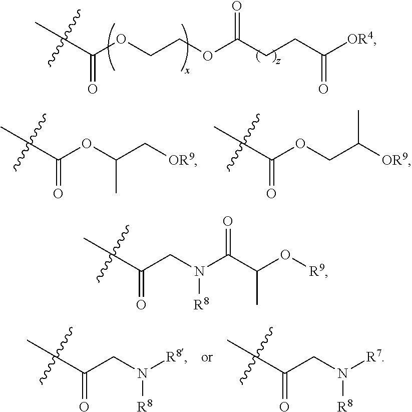

In one embodiment, R⁷ is —C(O)R⁴ and R⁴ is methyl.

In one embodiment, R^{20a} is

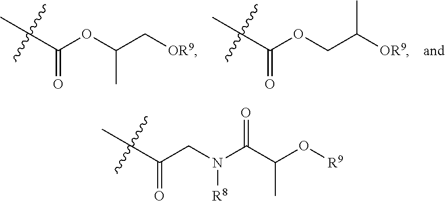

### and R⁹ is

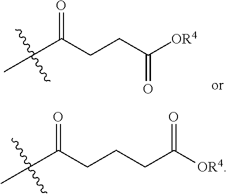

In one embodiment, R⁹ is

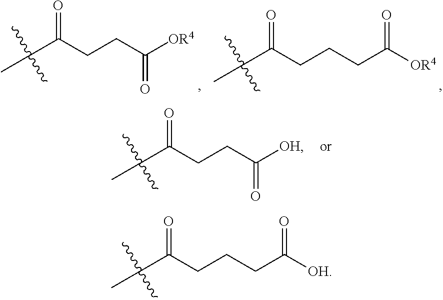

In one embodiment, z is an integer selected from 0, 1, 2, 3, 4, 5, and 6. In one embodiment, z is an integer selected from 1, 2, and 3.

In one embodiment, when R^{20a} is

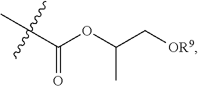

### R^{20a} is

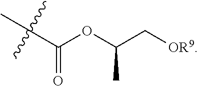

In one embodiment, when R^{20a} is

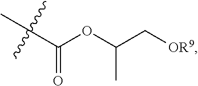

### R^{20a} is

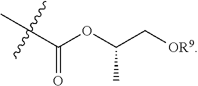

In one embodiment, when R^{20a} is

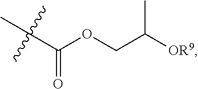

### R^{20a} is

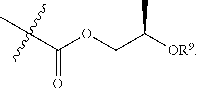

In one embodiment, when R^{20a} is

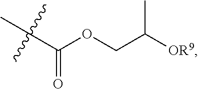

### R^{20a} is

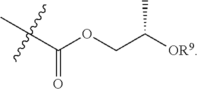

In one embodiment, when R^{20b} is

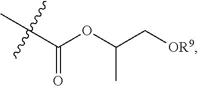

### R^{20b} is

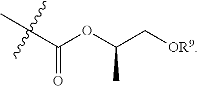

In one embodiment, when R^{20b} is

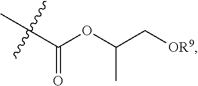

### R^{20b} is

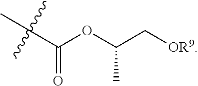

In one embodiment, when R^{20b} is

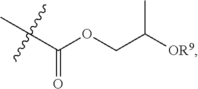

### R^{20b} is

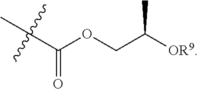

In one embodiment, when R^{20b} is

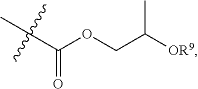

### R^{20b} is

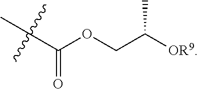

For example, Compound 67-7 is drawn as

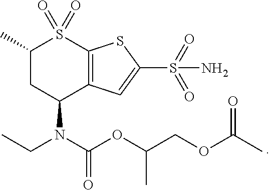 [SMILES: CCN(C(=O)OC(C)COC(C)=O)[C@H]1C[C@H](C)S(=O)(=O)c2sc(S(N)(=O)=O)cc21]

In one embodiment, Compound 67-7 is

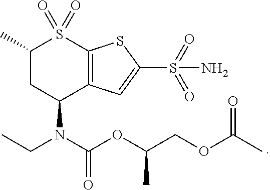 [SMILES: CCN(C(=O)O[C@H](C)COC(C)=O)[C@H]1C[C@H](C)S(=O)(=O)c2sc(S(N)(=O)=O)cc21]

In one embodiment, Compound 67-7 is

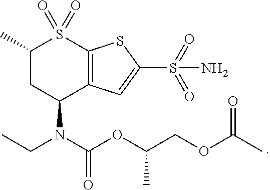 [SMILES: CCN(C(=O)O[C@@H](C)COC(C)=O)[C@H]1C[C@H](C)S(=O)(=O)c2sc(S(N)(=O)=O)cc21]

This disclosure also provides a compound of Formula (XII), Formula (XIII), Formula

(XIV), and Formula (XV):

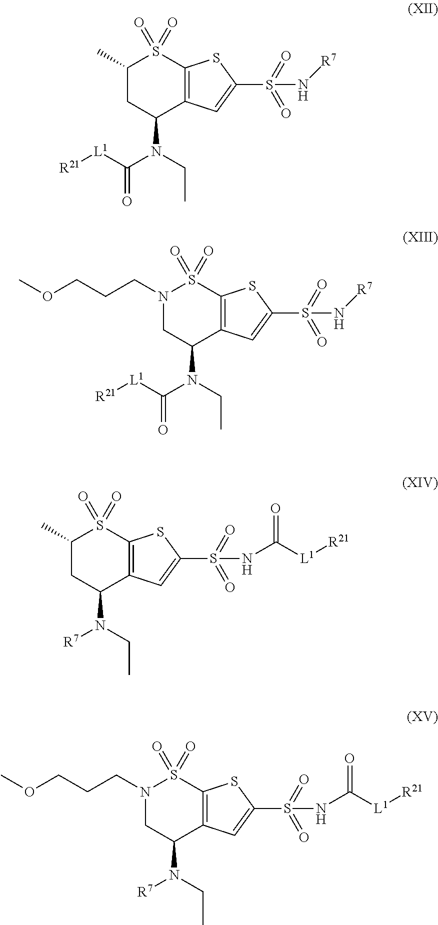

wherein

L¹ is selected from

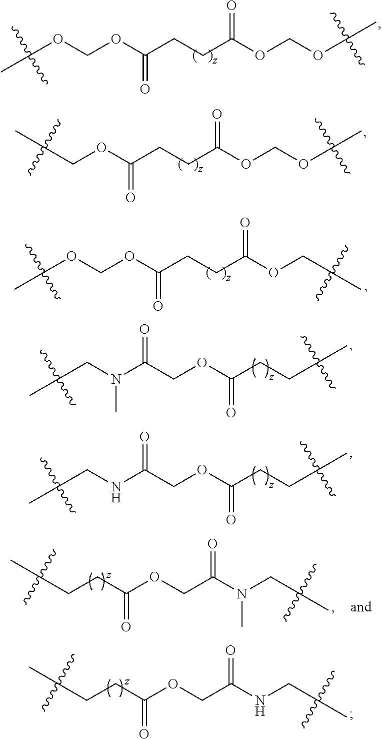

L² is selected from

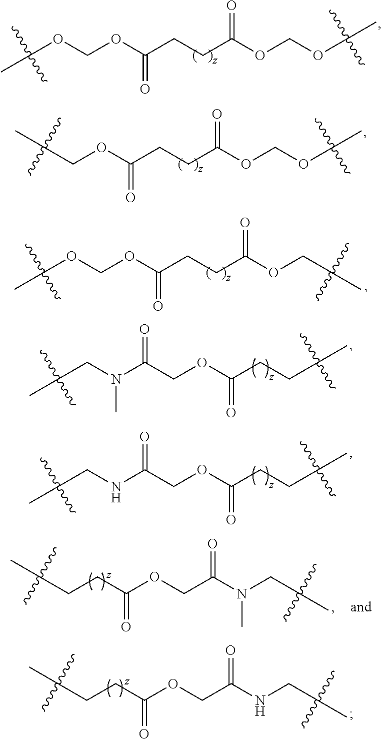

R²¹ is selected from

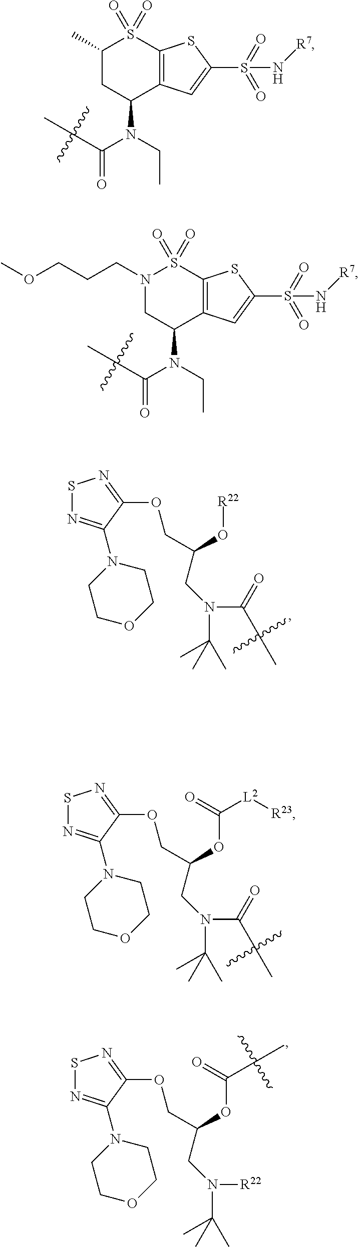

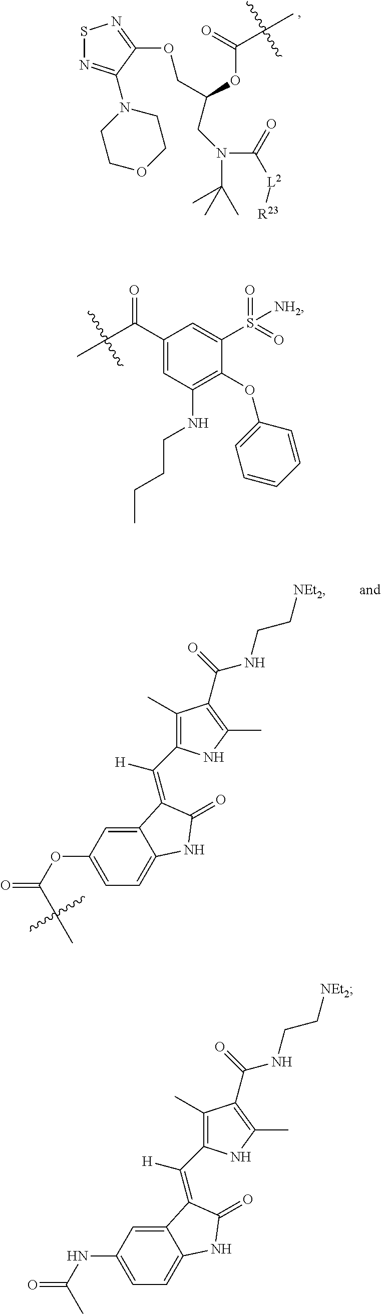

R²² is selected from

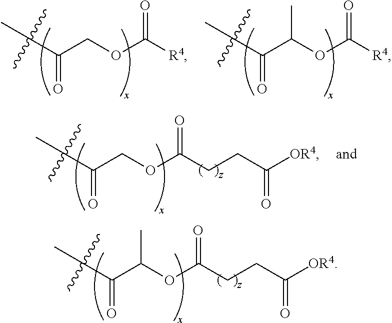

R²³ is selected from

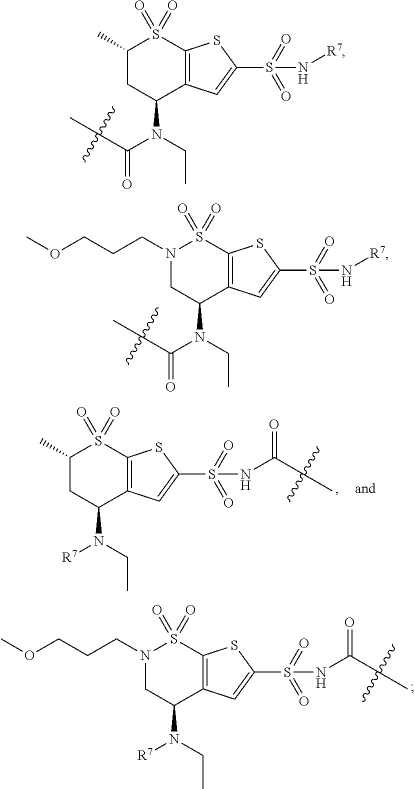

and

R⁴, R⁷, x, and z are defined herein.

Non-limiting examples of L¹ include

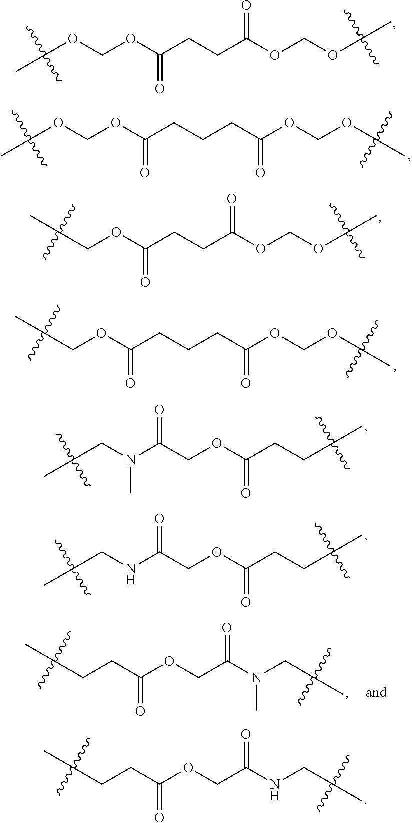

Non-limiting examples of L² include

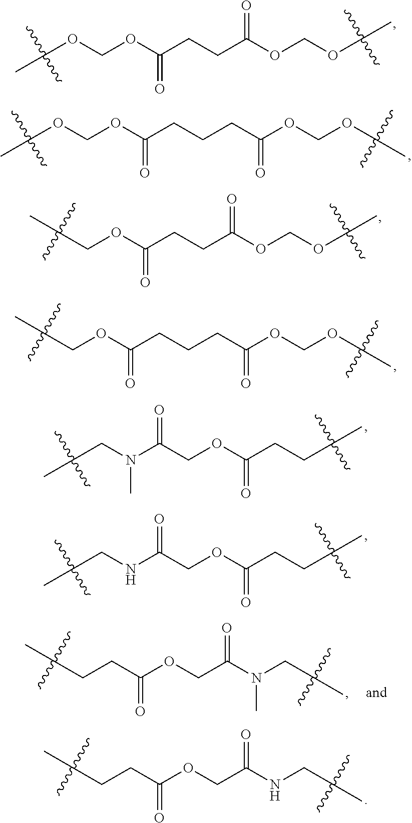

Non-limiting examples of R²² include

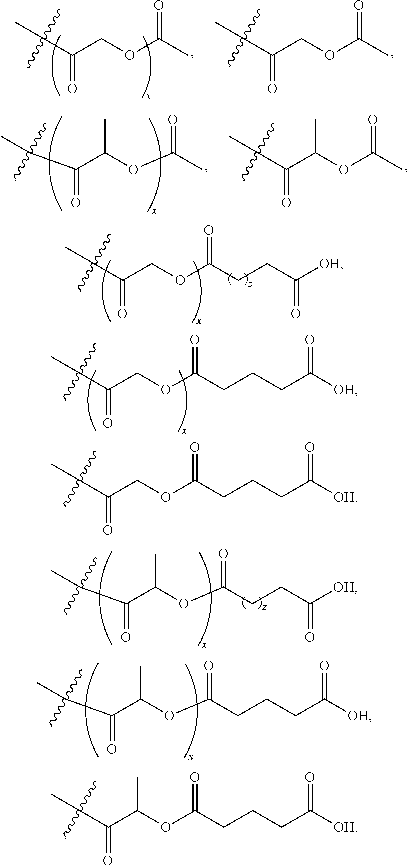

In one embodiment, x is an integer selected from 1, 2, 3, 4, 5, and 6. In one embodiment, x is an integer selected from 1, 2, and 3. In one embodiment, z is an integer selected from 1, 2, 3, 4, 5, and 6. In one embodiment, z is an integer selected from 1, 2, and 3.

In one embodiment, L¹ is

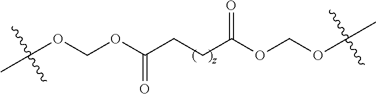

### and R²¹ is

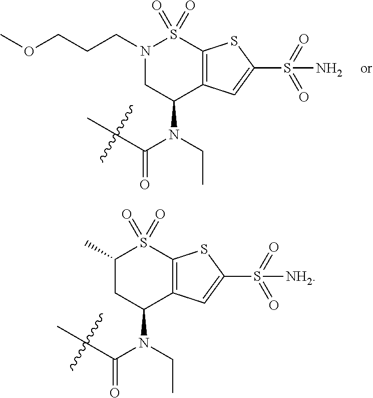

In one embodiment, R²¹ is

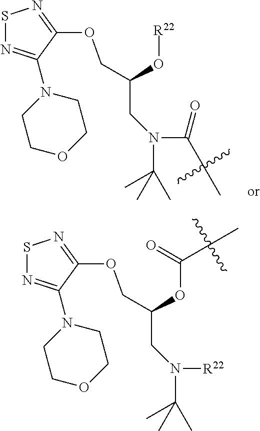

and R²² is selected from

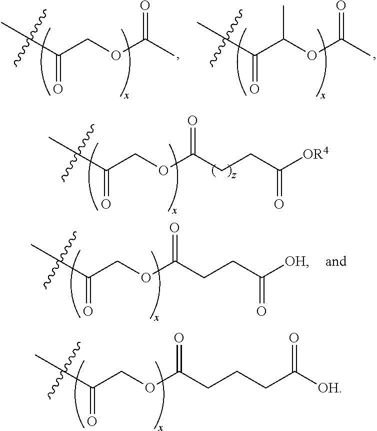

In one embodiment, L¹ is selected from

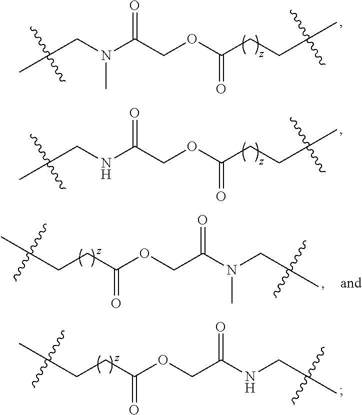

and

R²¹ is selected from

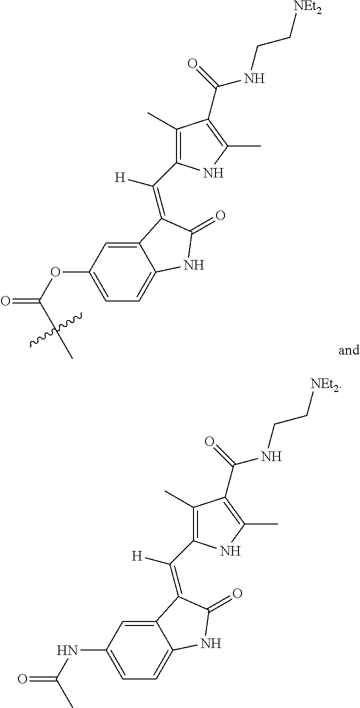

In a further embodiment, x is 1, 2, 3, 4, 5, or 6. In a further embodiment, xis 1.

Pharmaceutical compositions comprising a compound or salt of Formula I, Formula II, Formula III, Formula IV, Formula V, Formula VI, Formula VII, Formula VIII, Formula IX, Formula X, Formula XI, Formula XII, Formula XIII, Formula XIV, or Formula XV together with a pharmaceutically acceptable carrier are also disclosed.

Methods of treating or preventing ocular disorders, including glaucoma, a disorder mediated by carbonic anhydrase, a disorder mediated a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD), neovascular age-related macular degeneration (NVAMD), geographic atrophy, or diabetic retinopathy are disclosed comprising administering a therapeutically effective amount of a compound or salt or Formula I, Formula II, Formula III, Formula IV, Formula V, Formula VI, Formula VII, Formula VIII, Formula IX, Formula X, Formula XI, Formula XII, Formula XIII, Formula XIV, or Formula XV to a host, including a human, in need of such treatment.

In another embodiment, an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV is provided to decrease intraocular pressure (IOP) caused by glaucoma. In an alternative embodiment, the compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV can be used to decrease intraocular pressure (IOP), regardless of whether it is associated with glaucoma.

In one embodiment, the disorder is associated with an increase in intraocular pressure (IOP) caused by potential or previously poor patient compliance to glaucoma treatment. In yet another embodiment, the disorder is associated with potential or poor neuroprotection through neuronal nitric oxide synthase (NOS). The active compound or its salt or prodrug provided herein may thus dampen or inhibit glaucoma in a host, by administration of an effective amount in a suitable manner to a host, typically a human, in need thereof.

Methods for the treatment of a disorder associated with glaucoma, increased intraocular pressure (IOP), and optic nerve damage caused by either high intraocular pressure (IOP) or neuronal nitric oxide synthase (NOS) are provided that includes the administration of an effective amount of a compound Formula I, Formula II, Formula III, Formula IV, Formula V, Formula VI, Formula VII, Formula VIII, Formula IX, Formula X, Formula XI, Formula XII, Formula XIII, Formula XIV, or Formula XV or a pharmaceutically acceptable salt thereof, optionally in a pharmaceutically acceptable carrier are also disclosed.

Methods for the treatment of a disorder associated with age-related macular degeneration (AMD) and geographic atrophy are provided that includes the administration of an effective amount of a compound Formula I, Formula II, Formula III, Formula IV, Formula V, Formula VI, Formula VII, Formula VIII, Formula IX, Formula X, Formula XI, Formula XII, Formula XIII, Formula XIV, or Formula XV or a pharmaceutically acceptable salt thereof, optionally in a pharmaceutically acceptable carrier are also disclosed. In one embodiment, the age-related macular degeneration is wet age-related macular degeneration. In an alternative embodiment, the age-related macular degeneration is neovascular age-related macular degeneration.

Methods for treatment of a disorder mediated by a carbonic anhydrase are provided to treat a patient in need thereof wherein a prodrug of a carbonic anhydrase inhibitor as described herein is provided.

The present invention includes at least the following features:
(a) a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV as described herein, or a pharmaceutically acceptable salt or prodrug thereof (each of which and all subgenuses and species thereof are considered individually and specifically described);
(b) a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV as described herein, or a pharmaceutically acceptable salt or prodrug thereof, for use in treating or preventing an ocular disorder as further described herein;
(c) a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV as described herein, or a pharmaceutically acceptable salt or prodrug thereof for use in treating or preventing disorders related to an ocular disorder such as glaucoma, a disorder mediated by carbonic anhydrase, a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD), neovascular age-related macular degeneration (NVAMD), geographic atrophy or diabetic retinopathy;
(d) use of a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or a pharmaceutically acceptable salt or prodrug thereof in the manufacture of a medicament for use in treating or preventing glaucoma and disorders involving increased intraocular pressure (IOP) or nerve damage related to either IOP or nitric oxide synthase (NOS) and other disorders described further herein;
(e) use of a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or a pharmaceutically acceptable salt or prodrug thereof in the manufacture of a medicament for use in treating or preventing age-related macular degeneration (AMD) and other disorders described further herein;
(f) a process for manufacturing a medicament intended for the therapeutic use for treating or preventing glaucoma and disorders involving nerve damage related to both (IOP) and nitric oxide synthase (NOS) and other disorders described further herein characterized in that a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV as described herein is used in the manufacture;
(g) a pharmaceutical formulation comprising an effective host-treating amount of the a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or a pharmaceutically acceptable salt or prodrug thereof together with a pharmaceutically acceptable carrier or diluent;
(h) a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV as described herein in substantially pure form, (e.g., at least 90 or 95%);
(i) processes for the manufacture of a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or a pharmaceutically acceptable salt or prodrug thereof;
(j) processes for the preparation of therapeutic products including drug delivery agents that contain an effective amount a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV as described herein;
(k) A polymeric microparticle comprising Compound 1-1, Compound 2-1, Compound 3-1, Compound 16-2, Compound 25-1, or Compound 26-1 or a pharmaceutically acceptable salt thereof encapsulated in a blend of one or more hydrophobic polymer and an amphiphilic polymer wherein the loop diuretic is released for at least 1 month.
(l) Compound 1-1, Compound 2-1, Compound 3-1, Compound 16-2, Compound 25-1, or Compound 26-1 or a pharmaceutically acceptable salt thereof for use in treating a ocular disorder as further described herein wherein the compound is administered via intravitreal, intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, or tear duct injection;

### DETAILED DESCRIPTION

I. Terminology

The presently disclosed subject matter may be embodied in many different forms and should not be construed as limited to the embodiments set forth herein. Indeed, many modifications and other embodiments of the presently disclosed subject matter will come to mind for one skilled in the art to which the presently disclosed subject matter pertains having the benefit of the teachings presented in the descriptions included herein. Therefore, it is to be understood that the presently disclosed subject matter is not to be limited to the specific embodiments disclosed and that modifications and other embodiments are intended to be included within the scope of the disclosed subject matter.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Compounds are described using standard nomenclature. Unless defined otherwise, all technical and scientific terms used herein have the same meaning as is commonly understood by one of skill in the art to which this invention belongs.

The compounds in any of the Formulas described herein include enantiomers, mixtures of enantiomers, diastereomers, cis/trans isomers, tautomers, racemates and other isomers, such as rotamers, as if each is specifically described.

The compounds in any of the Formulas may be prepared by chiral or asymmetric synthesis from a suitable optically pure precursor or obtained from a racemate or mixture of enantiomers or diastereomers by any conventional technique, for example, by chromatographic resolution using a chiral column, TLC or by the preparation of diastereoisomers, separation thereof and regeneration of the desired enantiomer or diastereomer. See, e.g., “Enantiomers, Racemates and Resolutions,” by J. Jacques, A. Collet, and S. H. Wilen, (Wiley-Interscience, New York, 1981); S. H. Wilen, A. Collet, and J. Jacques, Tetrahedron, 2725 (1977); E. L. Eliel Stereochemistry of Carbon Compounds (McGraw-Hill, NY, 1962); and S. H. Wilen Tables of Resolving Agents and Optical Resolutions 268 (E. L. Eliel ed., Univ. of Notre Dame Press, Notre Dame, Ind., 1972, Stereochemistry of Organic Compounds, Ernest L. Eliel, Samuel H. Wilen and Lewis N. Manda (1994 John Wiley & Sons, Inc.), and Stereoselective Synthesis A Practical Approach, Mihály Nógrádi (1995 VCH Publishers, Inc., NY, N.Y.).

The terms “a” and “an” do not denote a limitation of quantity, but rather denote the presence of at least one of the referenced item. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and are independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed.

The present invention includes compounds of Formula I, Formula II, Formula III, Formula IV, Formula V, Formula VI, Formula VII, Formula VIII, Formula IX, Formula X, Formula XI, Formula XII, Formula XIII, Formula XIV, or Formula XV and the use of compounds with at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, phosphorus, fluorine, and chlorine, such as ²H, ³H, ¹¹C, ¹³C, ¹⁴C, ¹⁵N, ¹⁸F ³¹P, ³²P, ³⁵S, ³⁶CI, ¹²⁵I respectively. The invention includes isotopically modified compounds of Formula I, Formula II, Formula III, Formula IV, Formula V, Formula VI, Formula VII, Formula VIII, Formula IX, Formula X, Formula XI, Formula XII, Formula XIII, Formula XIV, or Formula XV. Isotopically labeled compounds of this invention and prodrugs thereof can generally be prepared by carrying out the procedures disclosed in the schemes or in the examples and preparations described below by substituting an isotopically labeled reagent for a non-isotopically labeled reagent.

By way of general example and without limitation, isotopes of hydrogen, for example, deuterium (²H) and tritium (³H) may be used anywhere in described structures that achieves the desired result. Alternatively or in addition, isotopes of carbon, e.g., ¹³C and ¹⁴C, may be used. In one embodiment, the isotopic substitution is deuterium for hydrogen at one or more locations on the molecule to improve the performance of the drug, for example, the pharmacodynamics, pharmacokinetics, biodistribution, half-life, stability, AUC, Tₘₐₓ, Cₘₐₓ, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, can be partial or complete. Partial deuterium substitution means that at least one hydrogen is substituted with deuterium. In certain embodiments, the isotope is 90, 95 or 99% or more enriched at any location of interest. In one embodiment deuterium is 90, 95 or 99% enriched at a desired location.

In one embodiment, the substitution of a hydrogen atom for a deuterium atom can be provided in any of A, QL¹, or L². In one embodiment, the substitution of a hydrogen atom for a deuterium atom occurs within an R group selected from any of R¹, R², R³, R⁴, R⁵, R⁶, R⁷, R⁸, R⁹, R¹⁰, R¹¹, R^{11′}, R¹², R¹³, R¹⁴, R¹⁵, R¹⁶, R¹⁷, R¹⁸, R¹⁹, R^{20a}, R^{20b}, R²¹, R²², and R²³ or an L group selected from L¹ and L². For example, when any of R groups are, or contain for example through substitution, methyl, ethyl, or methoxy, the alkyl residue may be deuterated (in non-limiting embodiments, CD₃, CH₂CD₃, CD₂CD₃, CDH₂, CD₂H, CD₃, CHDCH₂D, CH₂CD₃, CHDCHD₂, OCDH₂, OCD₂H, or OCD₃ etc.

The compound of the present invention may form a solvate with a solvent (including water). Therefore, in one embodiment, the invention includes a solvated form of the active compound. The term “solvate” refers to a molecular complex of a compound of the present invention (including salts thereof) with one or more solvent molecules. Examples of solvents are water, ethanol, dimethyl sulfoxide, acetone and other common organic solvents. The term “hydrate” refers to a molecular complex comprising a compound of the invention and water. Pharmaceutically acceptable solvates in accordance with the invention include those wherein the solvent may be isotopically substituted, e.g. D₂O, d₆-acetone, d₆-DMSO. A solvate can be in a liquid or solid form.

A dash (“—”) is defined by context and can in addition to its literary meaning indicate a point of attachment for a substituent. For example, —(C═O)NH₂ is attached through carbon of the keto (C═O) group. A dash (“—”) can also indicate a bond within a chemical structure. For example —C(O)—NH₂ is attached through carbon of the keto group which is bound to an amino group (NH₂).

An equal sign (“═”) is defined by context and can in addition to its literary meaning indicate a point of attachment for a substituent wherein the attachment is through a double bond. For example, ═CH₂ represents a fragment that is doubly bonded to the parent structure and consists of one carbon with two hydrogens bonded in a terminal fashion. ═CHCH₃ on the other hand represents a fragment that is doubly bonded to the parent structure and consists of two carbons. In the above example it should be noted that the stereoisomer is not delineated and that both the cis and trans isomer are independently represented by the group.

The term “substituted”, as used herein, means that any one or more hydrogens on the designated atom or group is replaced with a moiety selected from the indicated group, provided that the designated atom's normal valence is not exceeded. For example, when the substituent is oxo (i.e., ═O), then in one embodiment, two hydrogens on the atom are replaced. When an oxo group replaces two hydrogens in an aromatic moiety, the corresponding partially unsaturated ring replaces the aromatic ring. For example a pyridyl group substituted by oxo is a pyridone. Combinations of substituents and/or variables are permissible only if such combinations result in stable compounds or useful synthetic intermediates. In an alternative embodiment, the substituent is selected from —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, and unsubstituted heteroaryl.

A stable compound or stable structure refers to a compound with a long enough residence time to either be used as a synthetic intermediate or as a therapeutic agent, as relevant in context.

“Alkyl” is a straight chain or branched saturated aliphatic hydrocarbon group. In certain embodiments, the alkyl is C₁-C₂, C₁-C₃, C₁-C₆, or C₁-C₃₀ (i.e., the alkyl chain can be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbons in length). The specified ranges as used herein indicate an alkyl group with length of each member of the range described as an independent species. For example, C₁-C₆ alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙ alkyl is used herein in conjunction with another group, for example, (C₃-C₇cycloalkyl)C₀-C₄ alkyl, or —C₀-C₄alkyl(C₃-C₇cycloalkyl), the indicated group, in this case cycloalkyl, is either directly bound by a single covalent bond (C₀alkyl), or attached by an alkyl chain in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane and 2,3-dimethylbutane. In one embodiment, the alkyl group is optionally substituted as described above.

In an alternative embodiment, “cycloalkyl” is a saturated mono- or -multi-cycle hydrocarbon ring system. When composed of two or more rings, the rings may be joined together in a fused fashion. Non-limiting examples of typical cycloalkyl groups include cyclopropyl, cyclobutyl, cyclopentyl, cyclohexyl, cycloheptyl, and cyclooctyl.

“Alkenyl” is a straight or branched chain aliphatic hydrocarbon group having one or more carbon-carbon double bonds each of which is independently either cis or trans that may occur at a stable point along the chain. In one embodiment, the double bond in a long chain similar to a fatty acid has the stereochemistry as commonly found in nature. Non-limiting examples are C₂-C₃₀alkenyl, C₁₀-C₃₀alkenyl (i.e., having 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbons), and C₂-C₄alkenyl. The specified ranges as used herein indicate an alkenyl group having each member of the range described as an independent species, as described above for the alkyl moiety. Examples of alkenyl include, but are not limited to, ethenyl and propenyl. Alternative examples of alkenyl include C₂-C₈alkenyl, C₂-C₇alkenyl, C₂-C₆alkenyl, C₂-C₅alkenyl, and C₂-C₄alkenyl. In one embodiment, the alkenyl group is optionally substituted as described above.

“Alkynyl” is a straight or branched chain aliphatic hydrocarbon group having one or more carbon-carbon triple bonds that may occur at any stable point along the chain, for example, C₂-C₈alkynyl or C₁₀-C₃₀alkynyl (i.e., having 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl. In one embodiment, the alkynyl group is optionally substituted as described above.

“Alkylene” is a bivalent saturated hydrocarbon. Alkylenes, for example, can be a 1 to 8 carbon moiety, 1 to 6 carbon moiety, or an indicated number of carbon atoms, for example C₁-C₄alkylene, C₁-C₃alkylene, or C₁-C₂alkylene.

“Alkenylene” is a bivalent hydrocarbon having at least one carbon-carbon double bond. Alkenylenes, for example, can be a 2 to 8 carbon moiety, 2 to 6 carbon moiety, or an indicated number of carbon atoms, for example C₂-C₄alkenylene.

“Alkynylene” is a bivalent hydrocarbon having at least one carbon-carbon triple bond. Alkynylenes, for example, can be a 2 to 8 carbon moiety, 2 to 6 carbon moiety, or an indicated number of carbon atoms, for example C₂-C₄alkynylene.

“Alkenylalkynyl” in one embodiment is a bivalent hydrocarbon having at least one carbon-carbon double bond and at least one carbon-carbon triple bond. It will be recognized to one skilled in the art that the bivalent hydrocarbon will not result in hypervalency, for example, hydrocarbons that include —C═C≡C—C or —C≡C≡C—C. The hydrocarbons and must be stable. Alkenylalkynyls, for example, can be a 4 to 8 carbon moiety, 4 to 6 carbon moiety, or an indicated number of carbon atoms, for example C₄-C₆alkenylalkynyls.

“Alkoxy” is an alkyl group as defined above covalently bound through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “alkylthio” or a “thioalkyl” group is an alkyl group as defined above with the indicated number of carbon atoms covalently bound through a sulfur bridge (—S—). In one embodiment, the alkoxy group is optionally substituted as described above.

“Alkenyloxy” is an alkenyl group as defined covalently bound to the group it substitutes by an oxygen bridge (—O—).

“Aryl” indicates aromatic groups containing only carbon in the aromatic ring or rings. In one embodiment, the aryl groups contain 1 to 3 separate or fused rings and is 6 to about 14 or 18 ring atoms, without heteroatoms as ring members. When indicated, such aryl groups may be further substituted with carbon or non-carbon atoms or groups. Such substitution may include fusion to a 4 to 7-membered saturated cyclic group that optionally contains 1 or 2 heteroatoms independently chosen from N, O, B, and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described above. In one embodiment, aryl groups include, for example, dihydroindole, dihydrobenzofuran, isoindoline-1-one and indolin-2-one that can be optionally substituted.

The term “heterocycle,” or “heterocyclic ring” as used herein refers to a saturated or a partially unsaturated (i.e., having one or more double and/or triple bonds within the ring without aromaticity) carbocyclic radical of 3 to about 12, and more typically 3, 5, 6, 7 to 10 ring atoms in which at least one ring atom is a heteroatom selected from nitrogen, oxygen, phosphorus, silicon, boron and sulfur, the remaining ring atoms being C, where one or more ring atoms is optionally substituted independently with one or more substituents described above. A heterocycle may be a monocycle having 3 to 7 ring members (2 to 6 carbon atoms and 1 to 4 heteroatoms selected from N, O, P, and S) or a bicycle having 5 to 10 ring members (4 to 9 carbon atoms and 1 to 6 heteroatoms selected from N, O, P, and S), for example: a bicyclo [4,5], [5,5], [5,6], or [6,6] system. In one embodiment, the only heteroatom is nitrogen. In one embodiment, the only heteroatom is oxygen. In one embodiment, the only heteroatom is sulfur. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. Spiro moieties are also included within the scope of this definition. Examples of a heterocyclic group wherein 1 or 2 ring carbon atoms are substituted with oxo (═O) moieties are pyrimidinonyl and 1,1-dioxo-thiomorpholinyl. The heterocycle groups herein are optionally substituted independently with one or more substituents described herein.

“Heterocycloalkyl” is a saturated ring group with 1, 2, 3, or 4 heteroatoms independently chosen from N, S, and O, with remaining ring atoms being carbon. In a typical embodiment, nitrogen is the heteroatom. Monocyclic heterocycloalkyl groups typically have from 3 to about 8 ring atoms or from 4 to 6 ring atoms. Examples of heterocycloalkyl groups include morpholinyl, piperazinyl, piperidinyl, and pyrrolinyl.

“Heteroaryl” refers to a stable monocyclic, bicyclic, or multicyclic aromatic ring which contains from 1 to 3, or in some embodiments from 1, 2, or 3 heteroatoms selected from N, O, S, B or P with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B or P with remaining ring atoms being carbon. In one embodiment, the only heteroatom is nitrogen. In one embodiment, the only heteroatom is oxygen. In one embodiment, the only heteroatom is sulfur. Monocyclic heteroaryl groups typically have from 5, 6, or 7 ring atoms. In some embodiments bicyclic heteroaryl groups are 8- to 10-membered heteroaryl groups, that is, groups containing 8 or 10 ring atoms in which one 5, 6, or 7 member aromatic ring is fused to a second aromatic or non-aromatic ring. When the total number of S and O atoms in the heteroaryl group exceeds 1, these heteroatoms are not adjacent to one another. In one embodiment, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl.

In an alternative embodiment, when a term is used that include “alk” is should be understood that “cycloalkyl” or “carbocyclic” can be considered part of the definition, unless unambiguously excluded by context. For example and without limitation, the terms alkyl, alkenyl, alkynyl, alkoxy, alkanoyl, alkenloxy, haloalkyl, etc. can all be considered to include the cyclic forms of alkyl, unless unambiguously excluded by context.

The term “esterase” refers to an enzyme that catalyzes the hydrolysis of an ester. As used herein, the esterase can catalyze the hydrolysis of prostaglandins described herein. In certain instances, the esterase includes an enzyme that can catalyze the hydrolysis of amide bonds of prostaglandins.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

A “pharmaceutical composition” is a composition comprising at least one active agent, such as a compound or salt of Formula I, Formula II, Formula III, Formula IV, Formula V, Formula VI, Formula VII, Formula VIII, Formula IX, Formula X, Formula XI, Formula XII, Formula XIII, Formula XIV, or Formula XV and at least one other substance, such as a pharmaceutically acceptable carrier. “Pharmaceutical combinations” are combinations of at least two active agents which may be combined in a single dosage form or provided together in separate dosage forms with instructions that the active agents are to be used together to treat any disorder described herein.

A “pharmaceutically acceptable salt” includes a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salt can be prepared by reacting free acid forms of these compounds with a stoichiometric amount of the appropriate base (such as Na, Ca, Mg, or K hydroxide, carbonate, bicarbonate, or the like), or by reacting a free base form of the compound with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

Additional non-limiting examples of salts include 1-hydroxy-2-naphthoic acid, 2,2-dichloroacetic acid, 2-oxoglutaric acid, 4-acetamidobenzoic acid, 4-aminosalicylic acid, adipic acid, aspartic acid, benzenesulfonic acid, camphoric acid, camphor-10-sulfonic acid, capric acid, caproic acid, caprylic acid, carbonic acid, cinnamic acid, cyclamic acid, dodecylsulfuric acid, ethane-1,2-disulfonic acid, ethanesulfonic acid, formic acid, galactaric acid, gentisic acid, glucoheptonic acid, gluconic acid, glucuronic acid, glutaric acid, glycerophosphoric acid, hippuric acid, isobutyric acid, lactobionic acid, lauric acid, malonic acid, mandelic acid, naphthalene-1,5-disulfonic acid, naphthalene-2-sulfonic acid, nicotinic acid, nitric acid, oleic acid, palmitic acid, pyroglutamic acid, sebacic acid, thiocyanic acid, and undecylenic acid. Lists of additional suitable salts may be found, e.g., in Remington's Pharmaceutical Sciences, 17th ed., Mack Publishing Company, Easton, Pa., p. 1418 (1985).

The term “carrier” refers to a diluent, excipient, or vehicle with which an active compound is provided.

A “patient” or “host” or “subject” is typically a human, however, may be more generally a mammal. In an alternative embodiment it can refer to for example, a cow, sheep, goat, horses, dog, cat, rabbit, rat, mice, fish, bird and the like.

A “prodrug” as used herein, means a compound which when administered to a host in vivo is converted into a parent drug. As used herein, the term “parent drug” means the active form of the compounds that renders the biological effect to treat any of the disorders described herein, or to control or improve the underlying cause or symptoms associated with any physiological or pathological disorder described herein in a host, typically a human. Prodrugs can be used to achieve any desired effect, including to enhance properties of the parent drug or to improve the pharmaceutic or pharmacokinetic properties of the parent. Prodrug strategies exist which provide choices in modulating the conditions for in vivo generation of the parent drug, all of which are deemed included herein. Non-limiting examples of prodrug strategies include covalent attachment of removable groups, or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others. In certain aspects of the present invention, at least one hydrophobic group is covalently bound to the parent drug to slow release of the parent drug in vivo.

A “therapeutically effective amount” of a pharmaceutical composition/combination of this invention means an amount effective, when administered to a patient, to provide a therapeutic benefit such as an amelioration of symptoms of the selected disorder, typically an ocular disorder In certain aspects, the disorder is glaucoma, a disorder mediated by carbonic anhydrase, a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD), neovascular age-related macular degeneration (NVAMD), or diabetic retinopathy.

The term “polymer” as used herein includes oligomers.

II. Detailed Description of the Active Compounds

In certain embodiments, compounds for ocular delivery are provided that are lipophilic monoprodrugs of Sunitinib, Brinzolamide, or Dorzolamide covalently linked to a biodegradable oligomer, as described in more detail herein.

According to the present invention, compounds of Formula I, Formula II, Formula III, Formula IV, Formula V, Formula VI, Formula VII, Formula VIII, Formula IX, Formula X, Formula XI, Formula XII, Formula XIII, Formula XIV, or Formula XV are provided:

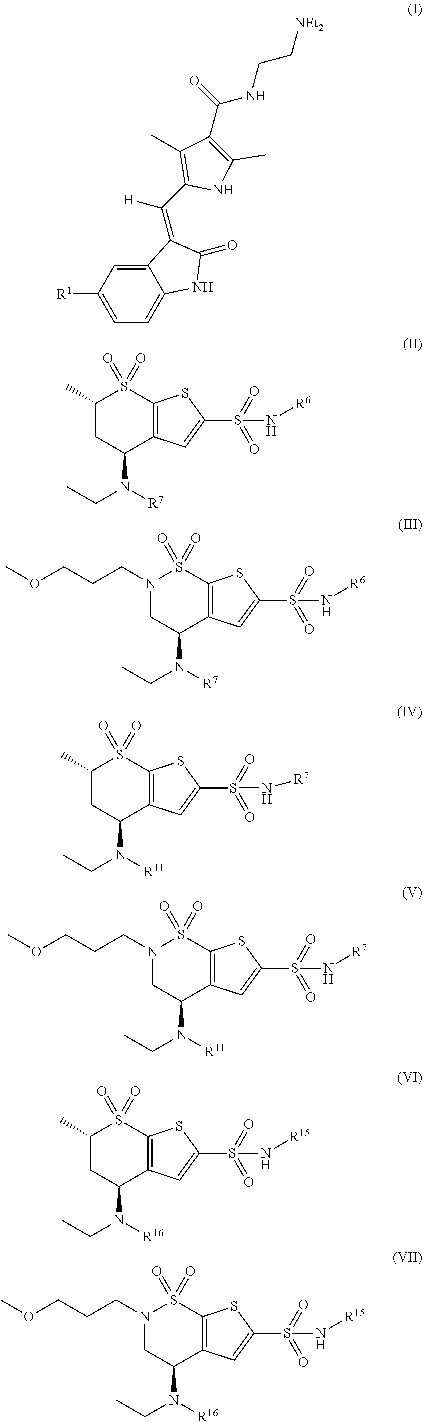

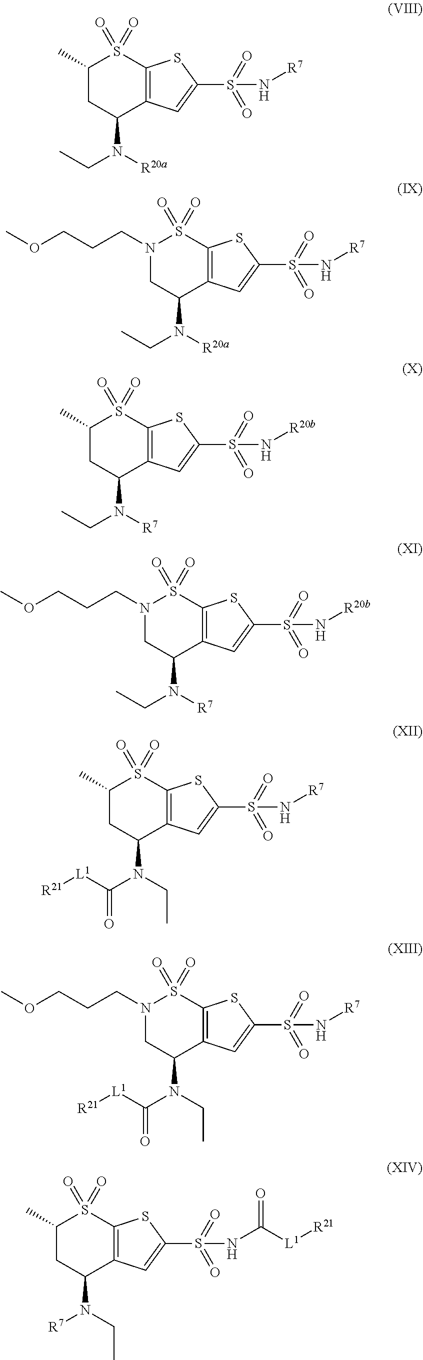

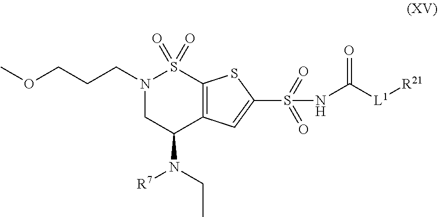

as well as the pharmaceutically acceptable salts and compositions thereof. Formula I is Sunitinib covalently bound to a hydrophobic moiety through an ether, ester, amine, or amide linkage that may be metabolized in the eye to afford Sunitinib or an active deriviative thereof. Formula II is Dorzolamide covalently bound to a hydrophobic moiety through a sulfonamide linkage that may be metabolized in the eye to afford Dorzolamide or an active deriviative thereof. Formula III is Brinzolamide covalently bound to a hydrophobic moiety through a sulfonamide linkage that may be metabolized in the eye to afford Brinzolamide or an active deriviative thereof. Formula IV is Dorzolamide covalently bound to a hydrophobic moiety through an amide linkage that may be metabolized in the eye to afford Dorzolamide or an active deriviative thereof. Formula V is Brinzolamide covalently bound to a hydrophobic moiety through an amide linkage that may be metabolized in the eye to afford Brinzolamide or an active deriviative thereof. Formula VI is Dorzolamide covalently bound to two hydrophobic moieties through an amide linkage and a sulfonamide linkage that may be metabolized in the eye to afford Dorzolamide or an active deriviative thereof. Formula VII is Brinzolamide covalently bound to two hydrophobic moieties through an amide linkage and a sulfonamide linkage that may be metabolized in the eye to afford Brinzolamide or an active deriviative thereof. Formula VIII is Dorzolamide covalently bound to a hydrophobic moiety through an amide linkage that may be metabolized in the eye to afford Dorzolamide or an active deriviative thereof. Formula IX is Brinzolamide covalently bound to a hydrophobic moiety through an amide linkage that may be metabolized in the eye to afford Brinzolamide or an active deriviative thereof. Formula X is Dorzolamide covalently bound to a hydrophobic moiety through a sulfonamide linkage that may be metabolized in the eye to afford Dorzolamide or an active deriviative thereof. Formula XI is Brinzolamide covalently bound to a hydrophobic moiety through a sulfonamide linkage that may be metabolized in the eye to afford Brinzolamide or an active deriviative thereof. Formula XII and Formula XIV is Dorzolamide covalently bound to another carbonic anhydrase inhibitor, a loop diuretic, a DLK inhibitor, or a β-blocker through a connecting fragment bound to both species that may be metabolized in the eye to afford both active species or active deriviatives thereof. Formula XIII and Formula XV is Brinzolamide covalently bound to another carbonic anhydrase inhibitor, a loop diuretic, a DLK inhibitor, or a β-blocker through a connecting fragment bound to both species that may be metabolized in the eye to afford both active species or active deriviatives thereof.

When a compound of Formula I is administered to a mammalian subject, typically a human, the prodrug may be cleaved to release the parent Sunitinib derivative or an active deriviative thereof. The active Sunitinib derivative is a phenol compound that has been demonstrated in the literature to be an active RTKI (Kuchar, M., et al. (2012). “Radioiodinated Sunitinib as a potential radiotracer for imaging angiogenesis-radiosynthesis and first radiopharmacological evaluation of 5-[125I]Iodo-Sunitinib.” Bioorg Med Chem Lett 22(8): 2850-2855. Formulations of Sunitinib for the treatment of ocular disorders and glaucoma have been described in WO2016/100392 and WO2016/100380, respectively.

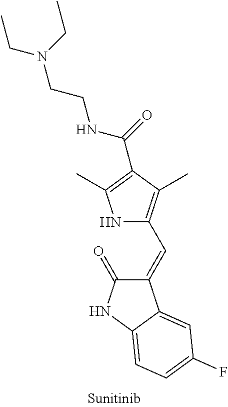 [SMILES: CCN(CC)CCNC(=O)c1c(C)[nH]c(/C=C2\C(=O)Nc3ccc(F)cc32)c1C]

The compounds, as described herein, may include, for example, prodrugs, which are hydrolysable to form Brinzolamide or Dorzolamide or an active deriviative thereof. Thus when a compound of Formula II, Formula III, Formula VI, Formula V, Formula VI, Formula VII, Formula VIII, Formula IX, Formula X, Formula XI, Formula XII, Formula XIII, Formula XIV, or Formula XV is administered to a mammalian subject, typically a human, the amide modifications or the sulfonamide modification may be cleaved to release Brinzolamide or Dorzolamide or an active deriviative thereof.

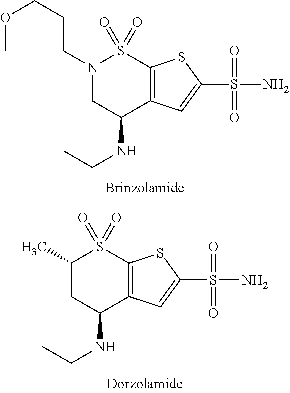 [SMILES: CCN[C@H]1CN(CCCOC)S(=O)(=O)c2sc(S(N)(=O)=O)cc21 ; CCN[C@H]1C[C@H](C)S(=O)(=O)c2sc(S(N)(=O)=O)cc21]

The compounds, as described herein, may include, for example, prodrugs, which are hydrolysable to release Timolol, Sunitinib, or Bumetanide or an active deriviative thereof in addition to Brinzolamide or Dorzolamide or an active deriviative thereof. Thus when a compound of Formula XII, Formula XIII, Formula XIV, or Formula XV is administered to a mammalian subject, typically a human, the prodrug may be cleaved to release Timolol, Sunitinib, or Bumetanide or an active deriviative thereof in addition to Brinzolamide or Dorzolamide or an active deriviative thereof.

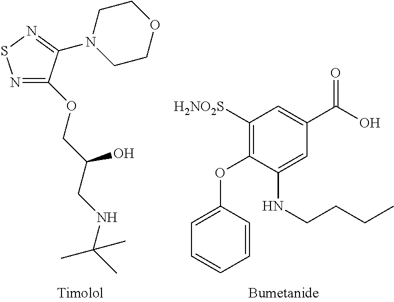 [SMILES: CC(C)(C)NC[C@H](O)COc1nsnc1N1CCOCC1 ; CCCCNc1cc(C(=O)O)cc(S(N)(=O)=O)c1Oc1ccccc1]

In certain embodiments, Compound 1-1, Compound 2-1, Compound 3-1, Compound 16-2, Compound 25-1, or Compound 26-1 are provided for ocular delivery as described in more detail herein.

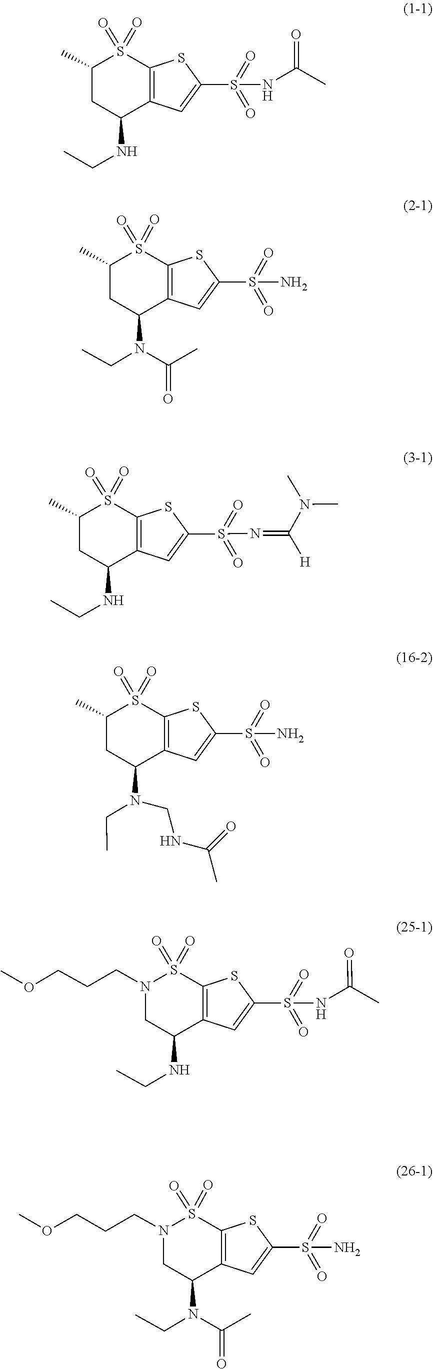

Compounds of the present invention with stereocenters may be drawn without stereochemistry for convenience. In general, unless otherwise indicated, the stereochemistry of the known drugs are as used on the approved commercial products. One skilled in the art will recognize that pure enantiomers and diastereomers can be prepared by methods known in the art. Examples of methods to obtain optically active materials include at least the following.

i) Physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) Simultaneous crystallization—a technique whereby the individual enantiomers are separately crystallized from a solution of the racemate, possible only if the latter is a conglomerate in the solid state;

iii) Enzymatic resolutions—a technique whereby partial or complete separation of a racemate by virtue of differing rates of reaction for the enantiomers with an enzyme;

iv) Enzymatic asymmetric synthesis—a synthetic technique whereby at least one step of the synthesis uses an enzymatic reaction to obtain an enantiomerically pure or enriched synthetic precursor of the desired enantiomer;

v) Chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) Diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) First- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) Kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) Enantiospecific synthesis from non-racemic precursors—a synthetic technique whereby the desired enantiomer is obtained from non-chiral starting materials and where the stereochemical integrity is not or is only minimally compromised over the course of the synthesis;

x) Chiral liquid chromatography—a technique whereby the enantiomers of a racemate are separated in a liquid mobile phase by virtue of their differing interactions with a stationary phase (including via chiral HPLC). The stationary phase can be made of chiral material or the mobile phase can contain an additional chiral material to provoke the differing interactions;

xi) Chiral gas chromatography—a technique whereby the racemate is volatilized and enantiomers are separated by virtue of their differing interactions in the gaseous mobile phase with a column containing a fixed non-racemic chiral adsorbent phase;

xii) Extraction with chiral solvents—a technique whereby the enantiomers are separated by virtue of preferential dissolution of one enantiomer into a particular chiral solvent;

xiii) Transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

xiv) Simulated moving bed chromatography, is used in one embodiment. A wide variety of chiral stationary phases are commercially available.

I. Pharmaceutical Preparations and Formulations

One embodiment provides pharmaceutical compositions that include the compounds described herein. In certain embodiments, the composition includes a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV in combination with a pharmaceutically acceptable carrier, excipient or diluent. In certain embodiments, the composition includes Compound 1-1, Compound 2-1, Compound 3-1, Compound 16-2, Compound 25-1, or Compound 26-1 in combination with a pharmaceutically acceptable carrier, excipient or diluent. In one embodiment, the composition is a pharmaceutical composition for treating an eye disorder or eye disease.

Compounds of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or pharmaceutically acceptable salts thereof can be delivered by any method known for ocular delivery. Methods include but are not limited to conventional (solution, suspension, emulsion, ointment, inserts and gels); vesicular (liposomes, niosomes, discomes and pharmacosomes), particulates (microparticles and nanoparticles), advanced materials (scleral plugs, gene delivery, siRNA and stem cells); and controlled release systems (implants, hydrogels, dendrimers, iontoporesis, collagen shields, polymeric solutions, therapeutic contact lenses, cyclodextrin carriers, microneedles and microemulsions).

In certain aspects, compounds of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or pharmaceutically acceptable salts thereof are administered via intravitreal, intrastromal, intracameral, sub-tenon, sub-retinal, retro-bulbar, peribulbar, suprachoroidal, choroidal, subchoroidal, conjunctival, episcleral, posterior juxtascleral, circumcorneal, or tear duct injection in combination with one or more pharmaceutically acceptable carriers. In another embodiment the selected compound is not administered topically. Representative carriers include solvents, diluents, pH modifying agents, preservatives, antioxidants, suspending agents, wetting agents, viscosity agents, tonicity agents, stabilizing agents, and combinations thereof.

The compounds of Formula I, Formula II, Formula III, Formula IV, Formula V, Formula VI, Formula VII, Formula VIII, Formula IX, Formula X, Formula XI, Formula XII, Formula XIII, Formula XIV, or Formula XV will preferably be formulated as a solution or suspension for injection to the eye. Pharmaceutical formulations for ocular administration are preferably in the form of a sterile aqueous solution. Acceptable solutions include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol. In some instances, the formulation is distributed or packaged in a liquid form. Alternatively, formulations for ocular administration can be packed as a solid, obtained, for example by lyophilization of a suitable liquid formulation. The solid can be reconstituted with an appropriate carrier or diluent prior to administration.

Solutions, suspensions, or emulsions for ocular administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for ocular administration may also contain one or more tonicity agents to adjust the isotonic range of the formulation. Suitable tonicity agents are well known in the art and some examples include glycerin, mannitol, sorbitol, sodium chloride, and other electrolytes.

Solutions, suspensions, or emulsions for ocular administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for ocular administration may also contain one or more excipients known art, such as dispersing agents, wetting agents, and suspending agents.

In one embodiment, a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or pharmaceutically acceptable salts thereof is administered in a dosage form that contains from about 1 μg to 10 mg, from about 1 μg to 1 mg, from about 1 μg to 100 μg, from about 1 μg to 50 μg, from about 1 μg to 10 μg, or from about 1 μg to 5 μg. In one embodiment, a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV is administered in a dosage form that contains up to about 1000, 950, 900, 850, 800, 750, 700, 650, 600, 550, 500, 450, 400, 350, 300, 250, 200, 150, 100, 90, 80, 70, 60, 50, 40, 30, 20, 15, 10, 5, or 1 μg. In another embodiment, a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV is administered in a dosage form that contains up to about 10, 9, 8, 7, 6, 5, 4, 3, 2, or 1 mg. In one embodiment, a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV is administered in a dosage form that contains at least about 1, 5, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, or 1000 μg. In another embodiment, a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV is administered in a dosage form that contains at least about 1, 2, 3, 4, 5, 6, 7, 8, 9, or 10 mg.

In certain aspects, a delivery system is used including but not limited to the following; i) a degradable polymeric composition; ii) a non-degradable polymeric composition; (iii) a gel, such as a hydrogel; (iv) a depot; (v) a particle containing a core; vi) a surface-coated particle; vii) a multi-layered polymeric or non-polymeric or mixed polymeric and non-polymeric particle; viii) a polymer blend and/or ix) a particle with a coating on the surface of the particle. The polymers can include, for example, hydrophobic regions. In some embodiments, at least about 30, 40 or 50% of the hydrophobic regions in the coating molecules have a molecular mass of least about 2 kDa. In some embodiments, at least about 30, 40 or 50% of the hydrophobic regions in the coating molecules have a molecular mass of least about 3 kDa. In some embodiments, at least about 30, 40 or 50% of the hydrophobic regions in the coating molecules have a molecular mass of least about 4 kDa. In some embodiments, at least about 30, 40 or 50% of the hydrophobic regions in the coating molecules have a molecular mass of least about 5 kDa. In certain embodiments, up to 5, 10, 20, 30, 40, 50, 60, 70, 80, 90 or even 95% or more of a copolymer or polymer blend consists of a hydrophobic polymer or polymer segment. In some embodiments, the polymeric material includes up to 2, 3, 4, 5, 6, 7, 8, 9, or 10% or more hydrophilic polymer. In one embodiment, the hydrophobic polymer is a polymer or copolymer of lactic acid or glycolic acid, including PLGA. In one embodiment, the hydrophilic polymer is polyethylene glycol. In certain embodiments a triblock polymer such as a Pluronic is used. The drug delivery system can be suitable for administration into an eye compartment of a patient, for example by injection into the eye compartment. In some embodiments, the core includes a biocompatible polymer. As used herein, unless the context indicates otherwise, “drug delivery system”, “carrier”, and “particle composition” can all be used interchangeably. In a typical embodiment this delivery system is used for ocular delivery.

The particle in the drug delivery system can be of any desired size that achieves the desired result. The appropriate particle size can vary based on the method of administration, the eye compartment to which the drug delivery system is administered, the therapeutic agent employed and the eye disorder to be treated, as will be appreciated by a person of skill in the art in light of the teachings disclosed herein. For example, in some embodiments the particle has a diameter of at least about 1 nm, or from about 1 nm to about 50 microns. The particle can also have a diameter of, for example, from about 1 nm to about 15, 16, 17, 18, 19, 2, 21, 22, 23, 24, 25, 26, 27, 28, 29 or 30 microns; or from about 10 nm to about less than 30, 35, 40, 45 or 50 microns; or from about 10 nm to about less than 28 microns; from about 1 nm to about 5 microns; less than about 1 nm; from about 1 nm to about 3 microns; or from about 1 nm to about 1000 nm; or from about 25 nm to about 75 nm; or from about 20 nm to less than or about 30 nm; or from about 100 nm to about 300 nm. In some embodiments, the average particle size can be about up to 1 nm, 10 nm, 25 nm, 30 nm, 50 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, or more. In some embodiments, the particle size can be about 100 microns or less, about 50 microns or less, about 30 microns or less, about 10 microns or less, about 6 microns or less, about 5 microns or less, about 3 microns or less, about 1000 nm or less, about 800 nm or less, about 600 nm or less, about 500 nm or less, about 400 nm or less, about 300 nm or less, about 200 nm or less, or about 100 nm or less. In some embodiments, the particle can be a nanoparticle or a microparticle. In some embodiments, the drug delivery system can contain a plurality of sizes particles. The particles can be all nanoparticles, all microparticles, or a combination of nanoparticles and microparticles.

When delivering the active material in a polymeric delivery composition, the active material can be distributed homogeneously, heterogeneously, or in one or more polymeric layers of a multi-layered composition, including in a polymer coated core or a bare uncoated core.

In some embodiments, the drug delivery system includes a particle comprising a core. In some embodiments a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV can be present in the core in a suitable amount, e.g., at least about 1% weight (wt), at least about 5% wt, at least about 10% wt, at least about 20% wt, at least about 30% wt, at least about 40% wt, at least about 50% wt, at least about 60% wt, at least about 70% wt, at least about 80% wt, at least about 85% wt, at least about 90% wt, at least about 95% wt, or at least about 99% wt of the core. In one embodiment, the core is formed of 100% wt of the pharmaceutical agent. In some cases, the pharmaceutical agent may be present in the core at less than or equal to about 100% wt, less than or equal to about 90% wt, less than or equal to about 80% wt, less than or equal to about 70% wt, less than or equal to about 60% wt, less than or equal to about 50% wt, less than or equal to about 40% wt, less than or equal to about 30% wt, less than or equal to about 20% wt, less than or equal to about 10% wt, less than or equal to about 5% wt, less than or equal to about 2% wt, or less than or equal to about 1% wt. Combinations of the above-referenced ranges are also possible (e.g., present in an amount of at least about 80% wt and less than or equal to about 100% wt). Other ranges are also possible.

In embodiments in which the core particles comprise relatively high amounts of a pharmaceutical agent (e.g., at least about 50% wt of the core particle), the core particles generally have an increased loading of the pharmaceutical agent compared to particles that are formed by encapsulating agents into polymeric carriers. This is an advantage for drug delivery applications, since higher drug loadings mean that fewer numbers of particles may be needed to achieve a desired effect compared to the use of particles containing polymeric carriers.

In some embodiments, the core is formed of a solid material having a relatively low aqueous solubility (i.e., a solubility in water, optionally with one or more buffers), and/or a relatively low solubility in the solution in which the solid material is being coated with a surface-altering agent. For example, the solid material may have an aqueous solubility (or a solubility in a coating solution) of less than or equal to about 5 mg/mL, less than or equal to about 2 mg/mL, less than or equal to about 1 mg/mL, less than or equal to about 0.5 mg/mL, less than or equal to about 0.1 mg/mL, less than or equal to about 0.05 mg/mL, less than or equal to about 0.01 mg/mL, less than or equal to about 1 μg/mL, less than or equal to about 0.1 μg/mL, less than or equal to about 0.01 μg/mL, less than or equal to about 1 ng/mL, less than or equal to about 0.1 ng/mL, or less than or equal to about 0.01 ng/mL at 25° C. In some embodiments, the solid material may have an aqueous solubility (or a solubility in a coating solution) of at least about 1 pg/mL, at least about 10 pg/mL, at least about 0.1 ng/mL, at least about 1 ng/mL, at least about 10 ng/mL, at least about 0.1 μg/mL, at least about 1 μg/mL, at least about 5 μg/mL, at least about 0.01 mg/mL, at least about 0.05 mg/mL, at least about 0.1 mg/mL, at least about 0.5 mg/mL, at least about 1.0 mg/mL, at least about 2 mg/mL. Combinations of the above-noted ranges are possible (e.g., an aqueous solubility or a solubility in a coating solution of at least about 10 pg/mL and less than or equal to about 1 mg/mL). Other ranges are also possible. The solid material may have these or other ranges of aqueous solubilities at any point throughout the pH range (e.g., from pH 1 to pH 14).

In some embodiments, the core may be formed of a material within one of the ranges of solubilities classified by the U.S. Pharmacopeia Convention: e.g., very soluble: >1,000 mg/mL; freely soluble: 100-1,000 mg/mL; soluble: 33-100 mg/mL; sparingly soluble: 10-33 mg/mL; slightly soluble: 1-10 mg/mL; very slightly soluble: 0.1-1 mg/mL; and practically insoluble: <0.1 mg/mL.

Although a core may be hydrophobic or hydrophilic, in many embodiments described herein, the core is substantially hydrophobic. “Hydrophobic” and “hydrophilic” are given their ordinary meaning in the art and, as will be understood by those skilled in the art, in many instances herein, are relative terms. Relative hydrophobicities and hydrophilicities of materials can be determined by measuring the contact angle of a water droplet on a planar surface of the substance to be measured, e.g., using an instrument such as a contact angle goniometer and a packed powder of the core material.

In some embodiments, the core particles described herein may be produced by nanomilling of a solid material (e.g., a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV) in the presence of one or more stabilizers/surface-altering agents. Small particles of a solid material may require the presence of one or more stabilizers/surface-altering agents, particularly on the surface of the particles, in order to stabilize a suspension of particles without agglomeration or aggregation in a liquid solution. In some such embodiments, the stabilizer may act as a surface-altering agent, forming a coating on the particle.

In a wet milling process, milling can be performed in a dispersion (e.g., an aqueous dispersion) containing one or more stabilizers (e.g., a surface-altering agent), a grinding medium, a solid to be milled (e.g., a solid pharmaceutical agent), and a solvent. Any suitable amount of a stabilizer/surface-altering agent can be included in the solvent. In some embodiments, a stabilizer/surface-altering agent may be present in the solvent in an amount of at least about 0.001% (wt or % weight to volume (w:v)), at least about 0.01, at least about 0.1, at least about 0.5, at least about 1, at least about 2, at least about 3, at least about 4, at least about 5, at least about 6, at least about 7, at least about 8, at least about 10, at least about 12, at least about 15, at least about 20, at least about 40, at least about 60, or at least about 80% of the solvent. In some cases, the stabilizer may be present in the solvent in an amount of about 100% (e.g., in an instance where the stabilizer/surface-altering agent is the solvent). In other embodiments, the stabilizer may be present in the solvent in an amount of less than or equal to about 100, less than or equal to about 80, less than or equal to about 60, less than or equal to about 40, less than or equal to about 20, less than or equal to about 15, less than or equal to about 12, less than or equal to about 10, less than or equal to about 8, less than or equal to about 7%, less than or equal to about 6%, less than or equal to about 5%, less than or equal to about 4%, less than or equal to about 3%, less than or equal to about 2%, or less than or equal to about 1% of the solvent. Combinations of the above-referenced ranges are also possible (e.g., an amount of less than or equal to about 5% and at least about 1% of the solvent). Other ranges are also possible. The particular range chosen may influence factors that may affect the ability of the particles to penetrate mucus such as the stability of the coating of the stabilizer/surface-altering agent on the particle surface, the average thickness of the coating of the stabilizer/surface-altering agent on the particles, the orientation of the stabilizer/surface-altering agent on the particles, the density of the stabilizer/surface altering agent on the particles, stabilizer/drug ratio, drug concentration, the size and polydispersity of the particles formed, and the morphology of the particles formed.

The compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV (or salt thereof) may be present in the solvent in any suitable amount. In some embodiments, the pharmaceutical agent (or salt thereof) is present in an amount of at least about 0.001% (wt % or % weight to volume (w:v)), at least about 0.01%, at least about 0.1%, at least about 0.5%, at least about 1%, at least about 2%, at least about 3%, at least about 4%, at least about 5%, at least about 6%, at least about 7%, at least about 8%, at least about 10%, at least about 12%, at least about 15%, at least about 20%, at least about 40%, at least about 60%, or at least about 80% of the solvent. In some cases, the pharmaceutical agent (or salt thereof) may be present in the solvent in an amount of less than or equal to about 100%, less than or equal to about 90%, less than or equal to about 80%, less than or equal to about 60%, less than or equal to about 40%, less than or equal to about 20%, less than or equal to about 15%, less than or equal to about 12%, less than or equal to about 10%, less than or equal to about 8%, less than or equal to about 7%, less than or equal to about 6%, less than or equal to about 5%, less than or equal to about 4%, less than or equal to about 3%, less than or equal to about 2%, or less than or equal to about 1% of the solvent. Combinations of the above-referenced ranges are also possible (e.g., an amount of less than or equal to about 20% and at least about 1% of the solvent). In some embodiments, the pharmaceutical agent is present in the above ranges but in w:v.

The ratio of stabilizer/surface-altering agent to pharmaceutical agent (or salt thereof) in a solvent may also vary. In some embodiments, the ratio of stabilizer/surface-altering agent to pharmaceutical agent (or salt thereof) may be at least 0.001:1 (weight ratio, molar ratio, or w:v ratio), at least 0.01:1, at least 0.01:1, at least 1:1, at least 2:1, at least 3:1, at least 5:1, at least 10:1, at least 25:1, at least 50:1, at least 100:1, or at least 500:1. In some cases, the ratio of stabilizer/surface-altering agent to pharmaceutical agent (or salt thereof) may be less than or equal to 1000:1 (weight ratio or molar ratio), less than or equal to 500:1, less than or equal to 100:1, less than or equal to 75:1, less than or equal to 50:1, less than or equal to 25:1, less than or equal to 10:1, less than or equal to 5:1, less than or equal to 3:1, less than or equal to 2:1, less than or equal to 1:1, or less than or equal to 0.1:1.

Combinations of the above-referenced ranges are possible (e.g. , a ratio of at least 5:1 and less than or equal to 50:1). Other ranges are also possible.

Stabilizers/surface-altering agents may be, for example, polymers or surfactants. Examples of polymers are those suitable for use in coatings, as described in more detail below. Non-limiting examples of surfactants include L-a-phosphatidylcholine (PC), 1,2-dipalmitoylphosphatidycholine (DPPC), oleic acid, sorbitan trioleate, sorbitan mono-oleate, sorbitan monolaurate, polyoxyethylene sorbitan monolaurate, polyoxyethylene sorbitan monooleate, natural lecithin, oleyl polyoxyethylene ether, stearyl polyoxyethylene ether, lauryl polyoxyethylene ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil, and sunflower seed oil. Derivatives of the above-noted compounds are also possible. Combinations of the above- noted compounds and others described herein may also be used as surface-altering agents in the inventive particles. As described herein, in some embodiments a surface-altering agent may act as a stabilizer, a surfactant, and/or an emulsifier. In some embodiments, the surface altering agent may aid particle transport in mucus.

It should be appreciated that while in some embodiments the stabilizer used for milling forms a coating on a particle surface, which coating renders particle mucus penetrating, in other embodiments, the stabilizer may be exchanged with one or more other surface-altering agents after the particle has been formed. For example, in one set of methods, a first stabilizer/surface-altering agent may be used during a milling process and may coat a surface of a core particle, and then all or portions of the first stabilizer/surface-altering agent may be exchanged with a second stabilizer/surface-altering agent to coat all or portions of the core particle surface. In some cases, the second stabilizer/surface-altering agent may render the particle mucus penetrating more than the first stabilizer/surface-altering agent. In some embodiments, a core particle having a coating including multiple surface-altering agents may be formed.

In other embodiments, core particles may be formed by a precipitation technique. Precipitation techniques (e.g., microprecipitation techniques, nanoprecipitation techniques) may involve forming a first solution comprising a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV and a solvent, wherein the material is substantially soluble in the solvent. The solution may be added to a second solution comprising another solvent in which the material is substantially insoluble, thereby forming a plurality of particles comprising the material. In some cases, one or more surface-altering agents, surfactants, materials, and/or bioactive agents may be present in the first and/or second solutions. A coating may be formed during the process of precipitating the core (e.g., the precipitating and coating steps may be performed substantially simultaneously). In other embodiments, the particles are first formed using a precipitation technique, following by coating of the particles with a surface-altering agent.

In some embodiments, a precipitation technique may be used to form particles (e.g., nanocrystals) of a salt of a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV. Generally, a precipitation technique involves dissolving the material to be used as the core in a solvent, which is then added to a miscible anti-solvent with or without excipients to form the core particle. This technique may be useful for preparing particles of pharmaceutical agents that are soluble in aqueous solutions (e.g., agents having a relatively high aqueous solubility). In some embodiments, pharmaceutical agents having one or more charged or ionizable groups can interact with a counter ion (e.g., a cation or an anion) to form a salt complex.

As described herein, in some embodiments, a method of forming a core particle involves choosing a stabilizer that is suitable for both nanomilling and for forming a coating on the particle and rendering the particle mucus penetrating. For example, as described in more detail below, it has been demonstrated that 200-500 nm nanoparticles of a model compound pyrene produced by nanomilling of pyrene in the presence of Pluronic® F127 resulted in particles that can penetrate physiological mucus samples at the same rate as well- established polymer-based MPP. Interestingly, it was observed that only a handful of stabilizers/surface-altering agents tested fit the criteria of being suitable for both nanomilling and for forming a coating on the particle that renders the particle mucus penetrating, as described in more detail below.

II. Description of Polymeric Delivery Materials

The particles of the drug delivery system can include a biocompatible polymer. As used herein, the term “biocompatible polymer” encompasses any polymer than can be administered to a patient without an unacceptable adverse effect to the patient.

Examples of biocompatible polymers include but are not limited to polystyrenes; poly(hydroxy acid); poly(lactic acid); poly(glycolic acid); poly(lactic acid-co-glycolic acid); poly(lactic-co-glycolic acid); poly(lactide); poly(glycolide); poly(lactide-co-glycolide); polyanhydrides; polyorthoesters; polyamides; polycarbonates; polyalkylenes; polyethylenes; polypropylene; polyalkylene glycols; poly(ethylene glycol); polyalkylene oxides; poly(ethylene oxides); polyalkylene terephthalates; poly(ethylene terephthalate); polyvinyl alcohols; polyvinyl ethers; polyvinyl esters; polyvinyl halides; poly(vinyl chloride); polyvinylpyrrolidone; polysiloxanes; poly(vinyl alcohols); poly(vinyl acetate); polyurethanes; co-polymers of polyurethanes; derivativized celluloses; alkyl cellulose; hydroxyalkyl celluloses; cellulose ethers; cellulose esters; nitro celluloses; methyl cellulose; ethyl cellulose; hydroxypropyl cellulose; hydroxy-propyl methyl cellulose; hydroxybutyl methyl cellulose; cellulose acetate; cellulose propionate; cellulose acetate butyrate; cellulose acetate phthalate; carboxylethyl cellulose; cellulose triacetate; cellulose sulfate sodium salt; polymers of acrylic acid; methacrylic acid; copolymers of methacrylic acid; derivatives of methacrylic acid; poly(methyl methacrylate); poly(ethyl methacrylate); poly(butylmethacrylate); poly(isobutyl methacrylate); poly(hexylmethacrylate); poly(isodecyl methacrylate); poly(lauryl methacrylate); poly(phenyl methacrylate); poly(methyl acrylate); poly(isopropyl acrylate); poly(isobutyl acrylate); poly(octadecyl acrylate); poly(butyric acid); poly(valeric acid); poly(lactide-co-caprolactone); copolymers of poly(lactide-co-caprolactone); blends of poly(lactide-co-caprolactone); hydroxyethyl methacrylate (HEMA); copolymers of HEMA with acrylate; copolymers of HEMA with polymethylmethacrylate (PMMA); polyvinylpyrrolidone/vinyl acetate copolymer (PVP/VA); acrylate polymers/copolymers; acrylate/carboxyl polymers; acrylate hydroxyl and/or carboxyl copolymers; polycarbonate-urethane polymers; silicone-urethane polymers; epoxy polymers; cellulose nitrates; polytetramethylene ether glycol urethane; polymethylmethacrylate-2-hydroxyethylmethacrylate copolymer; polyethylmethacrylate-2-hydroxyethylmethacrylate copolymer; polypropylmethacrylate-2-hydroxyethylmethacrylate copolymer; polybutylmethacrylate-2-hydroxyethylmethacryl ate copolymer; polymethylacrylate-2-hydroxyethylmethacrylate copolymer; polyethylacrylate-2-hydroxyethylmethacrylate copolymer; polypropylacrylate-2-hydroxymethacrylate copolymer; polybutylacrylate-2-hydroxyethylmethacrylate copolymer; copolymermethylvinylether maleicanhydride copolymer; poly (2-hydroxyethyl methacrylate) polymer/copolymer; acrylate carboxyl and/or hydroxy copolymer; olefin acrylic acid copolymer; ethylene acrylic acid copolymer; polyamide polymers/copolymers; polyimide polymers/copolymers; ethylene vinylacetate copolymer; polycarbonate urethane; silicone urethane; polyvinylpyridine copolymers; polyether sulfones; polygalactin, poly-(isobutyl cyanoacrylate), and poly(2-hydroxyethyl-L-glutamine); polydimethyl siloxane; poly(caprolactones); poly(ortho esters); polyamines; polyethers; polyesters; polycarbamates; polyureas; polyimides; polysulfones; polyacetylenes; polyethyeneimines; polyisocyanates; polyacrylates; polymethacrylates; polyacrylonitriles; polyarylates; and combinations, copolymers and/or mixtures of two or more of any of the foregoing. In some cases, the particle includes a hydrophobic material and at least one bioactive agent. In certain embodiments, the hydrophobic material is used instead of a polymer. In other embodiments, the hydrophobic material is used in addition to a polymer.

An active compound as described herein can be physically mixed in the polymeric material, including in an interpenetrating polymer network or can be covalently bound to the polymeric material

Linear, non-linear or linear multiblock polymers or copolymers can be used to form nanoparticles, microparticles, and implants (e.g., rods, discs, wafers, etc.) useful for the delivery to the eye. The polymers can contain one or more hydrophobic polymer segments and one or more hydrophilic polymer segments covalently connected through a linear link or multivalent branch point to form a non-linear multiblock copolymer containing at least three polymeric segments. The polymer can be a conjugate further containing one or more therapeutic, prophylactic, or diagnostic agents covalently attached to the one or more polymer segments. By employing a polymer-drug conjugate, particles can be formed with more controlled drug loading and drug release profiles. In addition, the solubility of the conjugate can be controlled so as to minimize soluble drug concentration and, therefore, toxicity.

The one or more hydrophobic polymer segments, independently, can be any biocompatible hydrophobic polymer or copolymer. In some cases, the one or more hydrophobic polymer segments are also biodegradable. Examples of suitable hydrophobic polymers include polyesters such as polylactic acid, polyglycolic acid, or polycaprolactone, polyanhydrides, such as polysebacic anhydride, and copolymers thereof. In certain embodiments, the hydrophobic polymer is a polyanhydride, such as polysebacic anhydride or a copolymer thereof. The one or more hydrophilic polymer segments can be any hydrophilic, biocompatible, non-toxic polymer or copolymer. The hydrophilic polymer segment can be, for example, a poly(alkylene glycol), a polysaccharide, poly(vinyl alcohol), polypyrrolidone, a polyoxyethylene block copolymer (PLURONIC®) or a copolymers thereof. In preferred embodiments, the one or more hydrophilic polymer segments are, or are composed of, polyethylene glycol (PEG).

WO 2016/100380A1 and WO 2016/100392 A1 describe certain Sunitinib delivery systems, which can also be used in the present invention to deliver the IOP lowering agents provided by the current invention, and as described further herein. For example, a process similar to that used in WO 2016/100380A1 and WO 2016/100392 A1 to prepare a polymeric Sunitinib drug formulation can be utilized: (i) dissolve or disperse the IOP lowering agent or its salt in an organic solvent; (ii) mix the solution/dispersion of step (i) with a polymer solution that has a viscosity of at least about 300 cPs (or perhaps at least about 350, 400, 500, 600, 700 or 800 or more cPs); (iii) mix the drug polymer solution/dispersion of step (ii) with an aqueous solution optionally with a surfactant or emulsifier, to form a solvent-laden encapsulated microparticle; and (iv) isolate the microparticles. Drug loading is also significantly affected by the method of making and the solvent used. For example, S/O/W single emulsion method will yield a higher loading than O/W single emulsion method even without control the acid value. In addition, W/O/W double emulsions have been shown to significantly improve drug loading of less hydrophobic salt forms over single O/W emulsions. The ratio of continuous phase to dispersed phase can also significantly alter the encapsulation efficiency and drug loading by modulation of the rate of particle solidification. The rate of polymer solidification with the evaporation of solvent affects the degree of porosity within microparticles. A large CP:DP ratio results in faster polymer precipitation, less porosity, and higher encapsulation efficiency and drug loading. However, decreasing the rate of evaporation of the solvent during particle preparation can also lead to improvements in drug loading of highly polar compounds. As the organic phase evaporates, highly polar compounds within the organic phase is driven to the surface of the particles resulting in poor encapsulation and drug loading. By decreasing the rate of solvent evaporation by decreasing the temperature or rate of stirring, encapsulation efficiency and % drug loading can be increased for highly polar compounds. These technologies can be used by one of skill in the art to deliver any of the active compounds as described generally in this specification.

U.S. Pat. No. 8,889,193 and PCT/US2011/026321 disclose, for example, a method for treating an eye disorder in a patient in need thereof, comprising administering into the eye, for example, by intravitreal injection into the vitreous chamber of the eye, an effective amount of a drug delivery system which comprises: (i) a microparticle including a core which includes the biodegradable polymer polylactide-co-glycolide; (ii) a coating associated with the core which is non-covalently associated with the microparticle particle; wherein the coating molecule has a hydrophilic region and a hydrophobic region, and wherein the hydrophilic region is polyethylene glycol; and (iii) a therapeutically effective amount of a therapeutic agent, wherein the drug delivery system provides sustained release of the therapeutic agent into the vitreous chamber over a period of time of at least three months; and wherein the vitreous chamber of the eye exhibits at least 10% less inflammation or intraocular pressure than if the particle were uncoated. In certain embodiments, the microparticle can be about 50 or 30 microns or less. The delivery system described in U.S. Pat. No. 8,889,193 and PCT/US2011/026321 can be used to deliver any of the active agents described herein.

In some embodiments, the drug delivery systems contain a particle with a coating on the surface, wherein the coating molecules have hydrophilic regions and, optionally, hydrophobic regions,

The drug delivery system can include a coating. The coating can be disposed on the surface of the particle, for example by bonding, adsorption or by complexation. The coating can also be intermingled or dispersed within the particle as well as disposed on the surface of the particle.

The homogeneous or heterogenous polymer or polymeric coating can be, for example, polyethylene glycol, polyvinyl alcohol (PVA), or similar substances. The coating can be, for example, vitamin E-PEG 1k or vitamin E-PEG 5k or the like. Vitamin E-PEG 5k can help present a dense coating of PEG on the surface of a particle. The coating can also include nonionic surfactants such as those composed of polyalkylene oxide, e.g., polyoxyethylene (PEO), also referred to herein as polyethylene glycol; or polyoxypropylene (PPO), also referred to herein as polypropylene glycol (PPG), and can include a copolymer of more than one alkylene oxide.

The polymer or copolymer can be, for example, a random copolymer, an alternating copolymer, a block copolymer or graft copolymer.

In some embodiments, the coating can include a polyoxyethylene-polyoxypropylene copolymer, e.g., block copolymer of ethylene oxide and propylene oxide. (i.e., poloxamers). Examples of poloxamers suitable for use in the present invention include, for example, poloxamers 188, 237, 338 and 407. These poloxamers are available under the trade name Pluronic® (available from BASF, Mount Olive, N.J.) and correspond to Pluronic® F-68, F-87, F-108 and F-127, respectively. Poloxamer 188 (corresponding to Pluronic® F-68) is a block copolymer with an average molecular mass of about 7,000 to about 10,000 Da, or about 8,000 to about 9,000 Da, or about 8,400 Da. Poloxamer 237 (corresponding to Pluronic® F-87) is a block copolymer with an average molecular mass of about 6,000 to about 9,000 Da, or about 6,500 to about 8,000 Da, or about 7,7000 Da. Poloxamer 338 (corresponding to Pluronic® F-108) is a block copolymer with an average molecular mass of about 12,000 to about 18,000 Da, or about 13,000 to about 15,000 Da, or about 14,600 Da. Poloxamer 407 (corresponding to Pluronic® F-127) is a polyoxyethylene-polyoxypropylene triblock copolymer in a ratio of between about E₁₀₁P₅₆E₁₀₁ to about E₁₀₆P₇₀E₁₀₆, or about E₁₀₁P₅₆E₁₀₁, or about E₁₀₆P₇₀E₁₀₆, with an average molecular mass of about 10,000 to about 15,000 Da, or about 12,000 to about 14,000 Da, or about 12,000 to about 13,000 Da, or about 12,600 Da. For example, the NF forms of poloxamers or Pluronic® polymers can be used.

In some embodiments, the polymer can be, for example Pluronic® P103 or Pluronic® P105. Pluronic® P103 is a block copolymer with an average molecular mass of about 3,000 Da to about 6,000 Da, or about 4,000 Da to about 6,000 Da, or about 4,950 Da. Pluronic® P105 is a block copolymer with an average molecular mass of about 5,000 Da to about 8,000 Da, or about 6,000 Da to about 7,000 Da, or about 6,500 Da.

In some embodiments, the polymer can have an average molecular weight of about 9,000 Da or greater, about 10,000 Da or greater, about 11,000 Da or greater or about 12,000 Da or greater. In exemplary embodiments, the polymer can have an average molecular weight of from about 10,000 to about 15,000 Da, or about 12,000 to about 14,000 Da, or about 12,000 to about 13,000 Da, or about 12,600 Da. In some embodiments, the polymer can be selected from Pluronic® P103, P105, F-68, F-87, F-108 and F-127, from Pluronic® P103, P105, F-87, F-108 and F-127, or from Pluronic® P103, P105, F-108 and F-127, or from Pluronic® P103, P105 and F-127. In some embodiments, the polymer can be Pluronic® F-127. In representative embodiments, the polymer is associated with the particles. For example, the polymer can be covalently attached to the particles. In representative embodiments, the polymer comprises polyethylene glycol, which is covalently attached to a selected polymer, yielding what is commonly referred to as a PEGylated particle.

In some embodiments, a coating is non-covalently associated with a core particle. This association can be held together by any force or mechanism of molecular interaction that permits two substances to remain in substantially the same positions relative to each other, including intermolecular forces, dipole-dipole interactions, van der Waals forces, hydrophobic interactions, electrostatic interactions and the like. In some embodiments, the coating is adsorbed onto the particle. According to representative embodiments, a non-covalently bound coating can be comprised of portions or segments that promote association with the particle, for example by electrostatic or van der Waals forces. In some embodiments, the interaction is between a hydrophobic portion of the coating and the particle. Embodiments include particle coating combinations which, however attached to the particle, present a hydrophilic region, e.g. a PEG rich region, to the environment around the particle coating combination. The particle coating combination can provide both a hydrophilic surface and an uncharged or substantially neutrally-charged surface, which can be biologically inert.

Suitable polymers for use according to the compositions and methods disclosed herein can be made up of molecules having hydrophobic regions as well as hydrophilic regions. Without wishing to be bound by any particular theory, when used as a coating, it is believed that the hydrophobic regions of the molecules are able to form adsorptive interactions with the surface of the particle, and thus maintain a non-covalent association with it, while the hydrophilic regions orient toward the surrounding, frequently aqueous, environment. In some embodiments the hydrophilic regions are characterized in that they avoid or minimize adhesive interactions with substances in the surrounding environment. Suitable hydrophobic regions in a coatings can include, for example, PPO, vitamin E and the like, either alone or in combination with each other or with other substances. Suitable hydrophilic regions in the coatings can include, for example, PEG, heparin, polymers that form hydrogels and the like, alone or in combination with each other or with other substances.

Representative coatings according to the compositions and methods disclosed herein can include molecules having, for example, hydrophobic segments such as PPO segments with molecular weights of at least about 1.8 kDa, or at least about 2 kDa, or at least about 2.4 kDa, or at least about 2.8 kDa, or at least about 3.2 kDa, or at least about 3.6 kDa, or at least about 4.0 kDa, or at least about 4.4 kDa, or at least about 4.8 kDa or at least about 5.2 kDa, or at least 5.6 kDa, or at least 6.0 kDa, or at least 6.4 kDa or more. In some embodiments, the coatings can have PPO segments with molecular weights of from about 1.8 kDa to about 10 kDa, or from about 2 kDa to about 5 kDa, or from about 2.5 kDa to about 4.5 kDa, or from about 2.5 kDa to about 3.5 kDa, or from about 3 kDa to about 6 kDa, or from about 3 kDa to about 5 kDa, or from about 4 kDa to about 6 kDa, or from about 4 kDa to about 7 kDa. In some embodiments, at least about 10%, or at least about 25%, or at least about 50%, or at least about 75%, or at least about 90%, or at least about 95%, or at least about 99% or more of the hydrophobic regions in these coatings have molecular weights within these ranges. In some embodiments, the coatings are biologically inert. Compounds that generate both a hydrophilic surface and an uncharged or substantially neutrally-charged surface can be biologically inert.

Representative coatings according to the compositions and methods disclosed herein can include molecules having, for example, hydrophobic segments such as PEG segments with molecular weights of at least about 1.8 kDa, or at least about 2 kDa, or at least about 2.4 kDa, or at least about 2.8 kDa, or at least about 3.2 kDa, or at least about 3.6 kDa, or at least about 4.0 kDa, or at least about 4.4 kDa, or at least about 4.8 kDa, or at least about 5.2 kDa, or at least 5.6 kDa, or at least 6.0 kDa, or at least 6.4 kDa or more. In some embodiments, the coatings can have PEG segments with molecular weights of from about 1.8 kDa to about 10 kDa, or from about 2 kDa to about 5 kDa, or from about 2.5 kDa to about 4.5 kDa, or from about 2.5 kDa to about 3.5 kDa. In some embodiments, at least about 10%, or at least about 25%, or at least about 50%, or at least about 75%, or at least about 90%, or at least about 95%, or at least about 99% or more of the hydrophobic regions in these coatings have molecular weights within these ranges. In some embodiments, the coatings are biologically inert. Compounds that generate both a hydrophilic surface and an uncharged or substantially neutrally-charged surface can be biologically inert.

Representative coatings according to the compositions and methods disclosed herein can include molecules having, for example, segments such as PLGA segments with molecular weights of at least about 4 kDa, or at least about 8 kDa, or at least about 12 kDa, or at least about 16 kDa, or at least about 20 kDa, or at least about 24 kDa, or at least about 28 kDa, or at least about 32 kDa, or at least about 36 kDa, or at least about 40 kDa, or at least about 44 kDa, of at least about 48 kDa, or at least about 52 kDa, or at least about 56 kDa, or at least about 60 kDa, or at least about 64 kDa, or at least about 68 kDa, or at least about 72 kDa, or at least about 76 kDa, or at least about 80 kDa, or at least about 84 kDa, or at least about 88 kDa or more. In some embodiments, at least about 10%, or at least about 25%, or at least about 50%, or at least about 75%, or at least about 90%, or at least about 95%, or at least about 99% or more of the regions in these coatings have molecular weights within these ranges. In some embodiments, the coatings are biologically inert. Compounds that generate both a hydrophilic surface and an uncharged or substantially neutrally-charged surface can be biologically inert.

In some embodiments, s coating can include, for example, one or more of the following: anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin), mucolytic agents, N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4, dornase alfa, neltenexine, erdosteine, various DNases including rhDNase, agar, agarose, alginic acid, amylopectin, amylose, beta-glucan, callose, carrageenan, cellodextrins, cellulin, cellulose, chitin, chitosan, chrysolaminarin, curdlan, cyclodextrin, dextrin, ficoll, fructan, fucoidan, galactomannan, gellan gum, glucan, glucomannan, glycocalyx, glycogen, hemicellulose, hydroxyethyl starch, kefiran, laminarin, mucilage, glycosaminoglycan, natural gum, paramylon, pectin, polysaccharide peptide, schizophyllan, sialyl lewis x, starch, starch gelatinization, sugammadex, xanthan gum, xyloglucan, L-phosphatidylcholine (PC), 1,2-dipalmitoylphosphatidycholine (DPPC), oleic acid, sorbitan trioleate, sorbitan monooleate, sorbitan monolaurate, polyoxyethylene (20) sorbitan monolaurate, polyoxyethylene (20) sorbitan monooleate, natural lecithin, oleyl polyoxyethylene (2) ether, stearyl polyoxyethylene (2) ether, polyoxyethylene (4) lauryl ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil, sunflower seed oil, lecithin, oleic acid, sorbitan trioleate, and combinations of two or more of any of the foregoing.

A particle-coating combinations can be made up of any combination of particle and coating substances disclosed or suggested herein. Examples of such combinations include, for example, polystyrene-PEG, or PLGA-Pluronic® F-127.

In one aspect of the present invention, an effective amount of an active compound as described herein is incorporated into a nanoparticle, e.g. for convenience of delivery and/or extended release delivery. The use of materials in nanoscale provides one the ability to modify fundamental physical properties such as solubility, diffusivity, blood circulation half-life, drug release characteristics, and/or immunogenicity. These nanoscale agents may provide more effective and/or more convenient routes of administration, lower therapeutic toxicity, extend the product life cycle, and ultimately reduce health-care costs. As therapeutic delivery systems, nanoparticles can allow targeted delivery and controlled release.

In another aspect of the present invention, the nanoparticle or microparticle is coated with a surface agent that facilitates passage of the particle through mucus. Said nanoparticles and microparticles have a higher concentration of surface agent than has been previously achieved, leading to the unexpected property of extremely fast diffusion through mucus. The present invention further comprises a method of producing said particles. The present invention further comprises methods of using said particles to treat a patient.

A number of companies have developed microparticles for treatment of eye disorders that can be used in conjunction with the present invention. For example, Allergan has disclosed a biodegradable microsphere to deliver a therapeutic agent that is formulated in a high viscosity carrier suitable for intraocular injection or to treat a non-ocular disorder (see U.S. publication 2010/0074957 and U.S. publication 2015/0147406). In one embodiment, the '957 application describes a biocompatible, intraocular drug delivery system that includes a plurality of biodegradable microspheres, a therapeutic agent, and a viscous carrier, wherein the carrier has a viscosity of at least about 10 cps at a shear rate of 0.1/second at 25° C. Allergan has also disclosed a composite drug delivery material that can be injected into the eye of a patient that includes a plurality of microparticles dispersed in a media, wherein the microparticles contain a drug and a biodegradable or bioerodible coating and the media includes the drug dispersed in a depot-forming material, wherein the media composition may gel or solidify on injection into the eye (see WO 2013/112434 A1, claiming priority to Jan. 23, 2012). Allergan states that this invention can be used to provide a depot means to implant a solid sustained drug delivery system into the eye without an incision. In general, the depot on injection transforms to a material that has a viscosity that may be difficult or impossible to administer by injection. In addition, Allergan has disclosed biodegradable microspheres between 40 and 200 μm in diameter, with a mean diameter between 60 and 150 μm that are effectively retained in the anterior chamber of the eye without producing hyperemia, see, US 2014/0294986. The microspheres contain a drug effective for an ocular condition with greater than seven day release following administration to the anterior chamber of the eye. The administration of these large particles is intended to overcome the disadvantages of injecting 1-30 μm particles which are generally poorly tolerated.

### Surface-Modified Solid Aggregating Microparticles

Surface-modified solid aggregating microparticles have been developed by Graybug Vision Inc. and are described in US 2017-0135960 and WO2017/083779. The surface-modified solid aggregating microparticles address the problem of intraocular therapy using small drug loaded particles (for example, 20 to 40 μm, 10 to 30, 20 to 30, or 25 to 30 μm average diameter, or for example, not greater than about 10, 20, 25, 26, 27, 28, 29, 30, 35, 40, 50, 60, or 70 μm average diameter (Dv)) that tend to disperse in the eye due to body movement and/or aqueous flow in the vitreous. The dispersed microparticles can cause vision disruption and aggravation from floaters, inflammation, etc. The surface-modified solid aggregating microparticles described herein aggregate in vivo to form at least one pellet of at least 500 μm to minimize vision disruption and inflammation. Further, the aggregated pellet of the surface treated microparticles is biodegradable so the aggregated pellet of the surface treated microparticles does not have to be surgically removed.

In one embodiment, an effective amount of a compound of Formula I, Formula II, Formula III, Formula IV, Formula V, Formula VI or Formula VII as described herein is encapsulated in a surface-modified solid aggregating microparticle as described in US 2017-0135960 or WO2017/083779. In one embodiment, an effective amount of Compound 1-1, Compound 2-1, Compound 3-1, Compound 16-2, Compound 25-1, or Compound 26-1 as described herein is encapsulated in a surface-modified solid aggregating microparticle as described in US 2017-0135960 or WO2017/083779.

The process for preparing a surface-modified solid aggregating microparticle includes
(a) a first step of preparing microparticles comprising one or more biodegradable polymers by dissolving or dispersing the polymer(s) and a therapeutically active agent selected from a compound of Formula I, Formula II, Formula III, Formula IV, Formula V, Formula VI or Formula VII, in one or more solvents to form a polymer and therapeutic agent solution or dispersion, mixing the polymer and the therapeutic agent solution or dispersion with an aqueous phase containing a surfactant to produce solvent-laden microparticles and then removing the solvent(s) to produce polymer microparticles that contain the therapeutic agent, polymer and surfactant; and
(ii) a second step of mildly treating the surface of microparticles of step (i) at a temperature at or below about 18, 15, 10, 8 or 5° C. optionally up to about 1, 2, 3, 4, 5, 10, 30, 40, 50, 60, 70, 80, 90 100, 11, 120 or 140 minutes with an agent that removes surface surfactant, surface polymer, or surface oligomer in a manner that does not significantly produce internal pores; and
(iii) isolating the surface treated microparticles.

In an alternative embodiment, the process for preparing a surface-modified aggregating microparticle includes
(a) a first step of preparing microparticles comprising one or more biodegradable polymers by dissolving or dispersing the polymer(s) and a therapeutically active agent selected from Compound 1-1, Compound 2-1, Compound 3-1, Compound 16-2, Compound 25-1, or Compound 26-1 in one or more solvents to form a polymer and therapeutic agent solution or dispersion, mixing the polymer and the therapeutic agent solution or dispersion with an aqueous phase containing a surfactant to produce solvent-laden microparticles and then removing the solvent(s) to produce polymer microparticles that contain the therapeutic agent, polymer and surfactant; and
(ii) a second step of mildly treating the surface of microparticles of step (i) at a temperature at or below about 18, 15, 10, 8 or 5° C. optionally up to about 1, 2, 3, 4, 5, 10, 30, 40, 50, 60, 70, 80, 90 100, 11, 120 or 140 minutes with an agent that removes surface surfactant, surface polymer, or surface oligomer in a manner that does not significantly produce internal pores; and
(iii) isolating the surface treated microparticles.

In certain embodiments step (ii) above is carried out at a temperature below 17° C., 15° C., 10° C., or 5° C. Further, step (iii) is optionally carried out at a temperature below 25° C., below 17° C., 15° C., 10° C., 8° C. or 5° C. Step (ii), for example, can be carried out for less than 8, less than 6, less than 4, less than 3, less than 2, or less than 1 minutes. In one embodiment, step (ii) is carried out for less than 60, 50, 40, 30, 20, or 10 minutes.

The process can be achieved in a continuous manufacturing line or via one step or in step-wise fashion. In one embodiment, wet biodegradable microparticles can be used without isolation to manufacture surface treated solid biodegradable microparticles. In one embodiment, the surface treated solid biodegradable microparticles do not significantly aggregate during the manufacturing process. In another embodiment, the surface treated solid biodegradable microparticles do not significantly aggregate when resuspended and loaded into a syringe. In some embodiments, the syringe is approximately 30, 29, 28, 27, 26 or 25 gauge, with either normal or thin wall.

A key aspect of the process is that the treatment, whether done in basic, neutral or acidic conditions, includes a selection of the combination of the time, temperature, pH agent and solvent that causes a mild treatment that does not significantly damage the particle in a manner that forms pores, holes or channels. Each combination of each of these conditions is considered independently disclosed as if each combination were separately listed.

In one embodiment, the surface treated solid biodegradable microparticles release about 1 to about 20 percent, about 1 to about 15 percent, about 1 to about 10 percent, or about 5 to 20 percent, for example, up to about 1, 5, 10, 15 or 20 percent, of the therapeutic agent over the first twenty-four hour period. In one embodiment, the surface treated solid biodegradable microparticles release less therapeutic agent in vivo in comparison to non-treated solid biodegradable microparticles over up to about 1, 2, 3, 4, 5, 6, 7 day or even up to about a 1, 2, 3, 4, or 5 month period. In one embodiment, the surface treated solid biodegradable microparticles induce less inflammation in vivo in comparison to non-treated solid biodegradable microparticles over the course of treatment.

In one embodiment, the process of manufacturing surface-modified solid aggregating microparticles includes using an agent that removes surface surfactant. Nonlimiting examples include for example, those selected from: aqueous acid, phosphate buffered saline, water, aqueous NaOH, aqueous hydrochloric acid, aqueous potassium chloride, alcohol or ethanol.

In one embodiment, the process of manufacturing surface-modified solid aggregating microparticles includes using an agent that removes surface surfactant which comprises, for example, a solvent selected from an alcohol, for example, ethanol; ether, acetone, acetonitrile, DMSO, DMF, THF, dimethylacetamide, carbon disulfide, chloroform, 1,1-dichloroethane, dichloromethane, ethyl acetate, heptane, hexane, methanol, methyl acetate, methyl t-butyl ether (MTBE), pentane, propanol, 2-propanol, toluene, N-methyl pyrrolidinone (NMP), acetamide, piperazine, triethylenediamine, diols, and CO₂.

The agent that removes the surface surfactant can comprise a basic buffer solution. Further, the agent that removes surface surfactant can comprises a base selected from sodium hydroxide, lithium hydroxide, potassium hydroxide, calcium hydroxide, magnesium hydroxide, lithium amide, sodium amide, barium carbonate, barium hydroxide, barium hydroxide hydrate, calcium carbonate, cesium carbonate, cesium hydroxide, lithium carbonate, magnesium carbonate, potassium carbonate, sodium carbonate, strontium carbonate, ammonia, methylamine, ethylamine, propylamine, isopropylamine, dimethylamine, diethylamine, dipropylamine, diisopropylamine, trimethylamine, triethylamine, tripropylamine, triisopropylamine, aniline, methylaniline, dimethylaniline, pyridine, azajulolidine, benzylamine, methylbenzylamine, dimethylbenzylamine, DABCO, 1,5-diazabicyclo[4.3.0]non-5-ene, 1,8-diazabicyclo[5.4.0]non-7-ene, 2,6-lutidine, morpholine, piperidine, piperazine, Proton-sponge, 1,5,7-Triazabicyclo[4.4.0]dec-5-ene, tripelennamine, ammonium hydroxide, triethanolamine, ethanolamine, and Trizma.

In one embodiment, the process of manufacturing surface-modified solid aggregating microparticles includes using an agent that removes surface surfactant, for example, those selected from the following: aqueous acid, phosphate buffered saline, water, or NaOH in the presence of a solvent such as an alcohol, for example, ethanol, ether, acetone, acetonitrile, DMSO, DMF, THF, dimethylacetamide, carbon disulfide, chloroform, 1,1-dichloroethane, dichloromethane, ethyl acetate, heptane, hexane, methanol, methyl acetate, methyl t-butyl ether (MTBE), pentane, ethanol, propanol, 2-propanol, toluene, N-methyl pyrrolidinone (NMP), acetamide, piperazine, triethylenediamine, diols, and CO₂.

In one embodiment, the agent that removes the surface surfactant can comprise an aqueous acid. The agent that removes the surface surfactant can comprise an acid derived from inorganic acids including, but not limited to, hydrochloric, hydrobromic, sulfuric, sulfamic, phosphoric, nitric and the like; or organic acids including, but not limited to, acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

In one embodiment, the agent that removes surface surfactant is not a degrading agent of the biodegradable polymer under the conditions of the reaction. The hydrophilicity of the microparticles can be decreased by removing surfactant.

In one embodiment, the process of manufacturing surface-modified solid aggregating microparticles comprises using an agent that removes surface surfactant that comprises a solvent selected from an alcohol, for example, ethanol, ether, acetone, acetonitrile, DMSO, DMF, THF, dimethylacetamide, carbon disulfide, chloroform, 1,1-dichloroethane, dichloromethane, ethyl acetate, heptane, hexane, methanol, methyl acetate, methyl t-butyl ether (MTBE), pentane, ethanol, propanol, 2-propanol, toluene, N-methyl pyrrolidinone (NMP), acetamide, piperazine, triethylenediamine, diols, and CO₂. In a typical embodiment the process of surface treating, comprises an agent that removes surface surfactant that comprises ethanol.

In some embodiments, the surface treatment is carried out at a temperature of not more than 5, 6, 7, 8, 9, 10, 11, 12, 13, 14, 15, 16, 17 or 18° C. at a reduced temperature of about 5 to about 18° C., about 5 to about 16° C., about 5 to about 15° C., about 0 to about 10° C., about 0 to about 8° C., or about 1 to about 5° C., about 5 to about 20° C., about 1 to about 10° C., about 0 to about 15° C., about 0 to about 10° C., about 1 to about 8° C., or about 1 to about 5° C. Each combination of each of these conditions is considered independently disclosed as if each combination were separately listed.

The pH of the surface treatment will of course vary based on whether the treatment is carried out in basic, neutral or acidic conditions. When carrying out the treatment in base, the pH may range from about 7.5 to about 14, including not more than about 8, 9, 10, 11, 12, 13 or 14. When carrying out the treatment in acid, the pH may range from about 6.5 to about 1, including not less than 1, 2, 3, 4, 5, or 6. When carrying out under neutral conditions, the pH may typically range from about 6.4 or 6.5 to about 7.4 or 7.5.

The treatment conditions should simply mildly treat the surface in a manner that allows the particles to remain as solid particles, be injectable without undue aggregation or clumping, and form at least one aggregate particle of at least 500 μm.

In one embodiment, the surface treatment includes treating microparticles with an aqueous solution of pH=6.6 to 7.4 or 7.5 and ethanol at a reduced temperature of about 1 to about 10° C., about 1 to about 15° C., about 5 to about 15° C., or about 0 to about 5° C. In one embodiment, the surface treatment includes treating microparticles with an aqueous solution of pH=6.6 to 7.4 or 7.5 and an organic solvent at a reduced temperature of about 0 to about 10° C., about 5 to about 8° C., or about 0 to about 5° C. In one embodiment, the surface treatment includes treating microparticles with an aqueous solution of pH=1 to 6.6 and ethanol at a reduced temperature of about 0 to about 10° C., about 0 to about 8° C., or about 0 to about 5° C. In one embodiment, the surface treatment includes treating microparticles with an organic solvent at a reduced temperature of about 0 to about 18° C., about 0 to about 16° C., about 0 to about 15° C., about 0 to about 10° C., about 0 to about 8° C., or about 0 to about 5° C. The decreased temperature of processing (less than room temperature, and typically less than 18° C.) assists to ensure that the particles are only “mildly” surface treated.

In one embodiment, a surface treated microparticle comprises a pharmaceutically active compound. The encapsulation efficiency of the pharmaceutically active compound in the microparticle can range widely based on specific microparticle formation conditions and the properties of the therapeutic agent, for example from about 20 percent to about 90 percent, about 40 percent to about 85 percent, about 50 percent to about 75 percent. In some embodiments, the encapsulation efficiency is for example, up to about 50, 55, 60, 65, 70, 75 or 80 percent.

The amount of pharmaceutical active compound in the surface treated microparticle is dependent on the molecular weight, potency, and pharmacokinetic properties of the pharmaceutical active compound.

In one embodiment, the pharmaceutically active compound is present in an amount of at least 1.0 weight percent to about 40 weight percent based on the total weight of the surface treated microparticle. In some embodiments, the pharmaceutically active compound is present in an amount of at least 1.0 weight percent to about 35 weight percent, at least 1.0 weight percent to about 30 weight percent, at least 1.0 weight percent to about 25 weight percent, or at least 1.0 weight percent to about 20 weight percent based on the total weight of the surface treated microparticle. Nonlimiting examples of weight of active material in the microparticle are at least about 5, 6, 7, 8, 9, 10, 11, 12, 13, 14 or 15% by weight. In one example, the microparticle has about 10% by weight of active compound.

In one embodiment, the microparticles have a mean size of about 25 μm to about 30 μm or 30 to 33 μm and a median size of about 31 μm to about 33 μm after surface treatment with approximately 0.0075 M NaOH/ethanol to 0.75 M NaOH/ethanol (30:70, v:v).

In one embodiment, the microparticles have a mean size of about 25 μm to about 30 μm or 30 to 33 μm and a median size of about 31 μm to about 33 μm after surface treatment with approximately 0.75 M NaOH/ethanol to 2.5 M NaOH/ethanol (30:70, v:v).

In one embodiment, the microparticles have a mean size of about 25 μm to about 30 μm or 30 to 33 μm and a median size of about 31 μm to about 33 μm after surface treatment with approximately 0.0075 M HCl/ethanol to 0.75 M NaOH/ethanol (30:70, v:v).

In one embodiment, the microparticles have a mean size of about 25 μm to about 30 μm or 30 to 33 μm and a median size of about 31 μm to about 33 μm after surface treatment with approximately 0.75 M NaOH/ethanol to 2.5 M HCl/ethanol (30:70, v:v).

In one embodiment, surface-modified solid aggregating microparticles that include at least one biodegradable polymer, wherein the surface-modified solid aggregating microparticles have a solid core, include a therapeutic agent selected from a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV, have a modified surface which has been treated under mild conditions at a temperature at or less than about 18° C. to remove surface surfactant, are sufficiently small to be injected in vivo, and are capable of aggregating in vivo to form at least one pellet of at least 500 μm in vivo to provide sustained drug delivery in vivo for at least one month, two months, three months, four months, five months, six months or seven months or more are provided. The surface modified solid aggregating microparticles are suitable, for example, for an intravitreal injection, implant, including an ocular implant, periocular delivery, or delivery in vivo outside of the eye.

In one embodiment, surface-modified solid aggregating microparticles that include at least one biodegradable polymer, wherein the surface-modified solid aggregating microparticles have a solid core, include a therapeutic agent selected from Compound 1-1, Compound 2-1, Compound 3-1, Compound 16-2, Compound 25-1, or Compound 26-1, have a modified surface which has been treated under mild conditions at a temperature at or less than about 18° C. to remove surface surfactant, are sufficiently small to be injected in vivo, and are capable of aggregating in vivo to form at least one pellet of at least 500 μm in vivo to provide sustained drug delivery in vivo for at least one month, two months, three months, four months, five months, six months or seven months or more are provided. The surface modified solid aggregating microparticles are suitable, for example, for an intravitreal injection, implant

Examples of solid cores included in the present invention include solid cores comprising a biodegradable polymer with less than 10 percent porosity, 8 percent porosity, 7 percent porosity, 6 percent porosity, 5 percent porosity, 4 percent porosity, 3 percent porosity, or 2 percent porosity. Porosity as used herein is defined by ratio of void space to total volume of the surface-modified solid aggregating microparticle.

In one embodiment, a method for the treatment of an ocular disorder is provided that includes administering to a host in need thereof mildly surface-modified solid aggregating microparticles that include an effective amount of a therapeutic agent selected from a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV, wherein the surface-modified solid aggregating microparticles are injected into the eye and aggregate in vivo to form at least one pellet of at least 500 μm that provides sustained drug delivery for at least approximately one, two, three, four, five, six or seven or more months in such a manner that the pellet stays substantially outside the visual axis so as not to significantly impair vision.

In yet another embodiment, a method for the treatment of an ocular disorder is provided that includes administering to a host in need thereof mildly surface-modified solid aggregating microparticles that include an effective amount of a therapeutic agent selected from Compound 1-1, Compound 2-1, Compound 3-1, Compound 16-2, Compound 25-1, or Compound 26-1, wherein the surface-modified solid aggregating microparticles are injected into the eye and aggregate in vivo to form at least one pellet of at least 500 μm that provides sustained drug delivery for at least approximately one, two, three, four, five, six or seven or more months in such a manner that the pellet stays substantially outside the visual axis so as not to significantly impair vision.

### Surface-Modified Solid Aggregating Microparticles Suspensions

The process and materials of surface-modification as described in US 2017-0135960 and WO2017/083779 provide acceptable aggregating microparticles in vivo, however, there are occasions when if surface-treated microparticles are overtreated (e.g., treated under strong chemical conditions or for an extended period of time), they may have a tendency to float upon injection into an aqueous solution with low viscosity (e.g., PBS buffer solution or sometimes vitreal fluid, wherein the viscosity may decrease with age of the patient), which is disadvantageous for forming a pellet that remains out of the visual axis. Since ocular disorders increase with age, it is important to provide a particle suspension that still aggregates to a pellet in lower viscosity vitreous fluid. Certain aspects of this invention address those certain situations, where a thin layer of air, air bubbles or gas generally can adhere to the surface of some microparticles and prevent the particles from being completely wetted. If this tiny layer of air or bubbles is high enough to create buoyancy, the microparticles will be less likely to aggregate to the desired pellet.

Therefore, in a further embodiment, the process for preparing a surface-modified solid aggregating microparticles can also include a fourth step, which is described in PCT/US18/32167 and U.S. Ser. No. 15/976,847 assigned to Graybug Vision. The fourth step includes:
(iv) subjecting the microparticles to at least one process selected from 1) vacuum treatment prior to lyophilization or other form of reconstitutable solidification, or after the step of reconstitution wherein the microparticles are suspended in a diluent and the suspension is placed under vacuum prior to use; 2) excipient addition, wherein an excipient is added prior to lyophilization; and 3) sonication, prior to lyophilization or other form of reconstitutable solidification, or after the step of reconstitution; 4) sealing the vial containing the dry powder of particles under vacuum, including but not limited to high vacuum; or 5) pre-wetting (i.e., resuspending) the surface-treated microparticles in a diluent for 2-24 hours before injecting into the eye, for example in a hyaluronic acid solution or other sterile solution suitable for ocular injection.

The process of step (iv) above can be carried out following isolation of the microparticles and/or upon reconstitution prior to injection.

In one non-limiting embodiment, a process for preparing a suspension comprising a microparticle and a pharmaceutically active compound as described herein encapsulated in the microparticle includes:
(a) preparing a solution or suspension (organic phase) comprising: (i) PLGA or PLA or PLA and PLGA, (ii) PLGA-PEG or PLA-PEG (iii) a pharmaceutically active compound as described herein and (iv) one or more organic solvents;
(b) preparing an emulsion in an aqueous polyvinyl alcohol (PVA) solution (aqueous phase) by adding the organic phase into the aqueous phase and mixing them until particle formation (for example at about 3,000 to about 10,000 rpm for about 1 to about 30 minutes);
(c) removing additional solvent as necessary using known techniques;
(d) centrifuging or causing the sedimentation of the microparticle that is loaded with a pharmaceutically active compound or prodrug thereof;
(e) optionally removing additional solvent and/or washing the microparticle loaded with the pharmaceutically active compound or prodrug thereof with water;
(f) filtering the microparticle loaded with pharmaceutically active compound or prodrug thereof to remove aggregates or particles larger than the desired size;
(g) optionally lyophilizing the microparticle comprising the pharmaceutically active compound and storing the microparticle as a dry powder in a manner that maintains stability for up to about 6, 8, 10, 12, 20, 22, or 24 months or more; and
(h) optionally improving the aggregation potential of the particles by subjecting the particles to at least one process selected from 1) vacuum treatment prior to step (g), or after reconstitution wherein the microparticles are suspended in a diluent and the suspension is placed under vacuum; 2) excipient addition, wherein an excipient is added prior to lyophilization; and 3) sonication prior to step (g), or during reconstitution wherein the microparticles are suspended in a diluent and sonicated; 4) sealing the vial containing the dry powder of particles under vacuum, including but not limited to high vacuum; or 5) pre-wetting (i.e., resuspending) the surface-treated microparticles in a diluent for 2-24 hours before injecting into the eye, for example in a hyaluronic acid solution or other sterile solution suitable for ocular injection.

In one embodiment, a process for preparing an improved lyophilized material or a suspension of microparticles following reconstitution includes suspending the particles in a diluent and subjecting the particles to vacuum treatment at a pressure of about less than about 500, 400, 300, 200, 150, 100, 75, 50, 40, 35, 34, 33, 32, 31, 30, 29, 28 or 25 Torr for a suitable amount of time to substantially remove air attached to the particles, which in some embodiments can be up to 3, 5, 8, 10, 20, 30, 40, 50, 60, 70, 80, or 90 minutes or up to 2, 3, 4, 5, or 6, 10, 15 or 24 or more hours. In one embodiment, the vacuum treatment is conducted with a VacLock syringe in a size of up to at least 10, 20, 30, or 60 mL.

In certain non-limiting embodiments, the microparticles are vacuumed at a strength of less than 40 Torr for about 3, 5, 8, 10, 20, 30, 45, 60, 75, or 90 minutes. In certain non-limiting embodiments, the microparticles are vacuumed at a strength less than 40 Torr from about 1 to 90 minutes, from about 1 to 60 minutes, from about 1 to 45 minutes, from about 1 to 30 minutes, from about 1 to 15 minutes, or from about 1 to 5 minutes.

In certain embodiments, the diluent for suspending particles is ProVisc. In some embodiments, the microparticles are diluted from about 10-fold to about 40-fold, from about 15-fold to about 35-fold, or from about 20-fold to about 25-fold. In some embodiments, the diluent for suspending particles is a 10×-diluted ProVisc (0.1% HA in PBS) solution, a 20×-diluted ProVisc (0.05% HA in PBS) solution, or a 40×-diluted ProVisc (0.025% HA in PBS) solution. In some embodiment, the particles are suspended in the diluent at a concentration of at least about 100 mg/mL, 200 mg/mL, 300 mg/mL, 400 mg/mL, or 500 mg/mL.

### Vacuum Treatment

In one embodiment, the process for providing an improved microparticle suspension prior to injection includes vacuum treatment wherein the particles are suspended in a diluent and subjected to negative pressure to remove unwanted air at the surface of the microparticles. Nonlimiting examples of the negative pressure can be about or less than 300, 200, 100, 150, 145, 143, 90, 89, 88, 87, 86, 85, 75, 50, 35, 34, 33, 32, 31, or 30 Torr for any appropriate time to achieve the desired results, including but not limited to 120, 110, 100, 90, 80, 70, 60, 50, 40, 30, 20, 10, 8, 5, or 3 minutes.

In one embodiment, microparticles are stored under negative pressure following the manufacturing and isolation process, wherein negative pressure is defined as any pressure lower than the pressure of ambient room temperature (approximately 760 Torr). In one embodiment, the microparticles are stored at a pressure of less than about 700 Torr, 550 Torr, 500 Torr, 450 Torr, 400 Torr, 350 Torr, 300 Torr, 250 Torr, 200 Torr, 150 Torr, 100 Torr, 90 Torr, 80 Torr, 60 Torr, 40 Torr, 35 Torr, 32 Torr, 30 Torr, or 25 Torr following the manufacturing and isolation process. In one embodiment, the microparticles are stored at a pressure of about 500 Torr to about 25 Torr following the manufacturing and isolation process. In one embodiment, the microparticles are stored at a pressure of about 300 Torr to about 25 Torr following the manufacturing and isolation process. In one embodiment, the microparticles are stored at a pressure of about 100 Torr to about 25 Torr following the manufacturing and isolation process. In one embodiment, the microparticles are stored at a pressure of about 90 Torr to about 25 Torr following the manufacturing and isolation process. In one embodiment, the microparticles are stored at a pressure of about 50 Torr to about 25 Torr following the manufacturing and isolation process. In one embodiment, the microparticles are stored at a pressure of about 40 Torr to about 25 Torr following the manufacturing and isolation process. In one embodiment, the microparticles are stored at a pressure of about 35 Torr to about 25 Torr following the manufacturing and isolation process. In a further embodiment, the microparticles are stored at a temperature of between about 2-8° C. at a pressure that is less than about 700, 550, 500, 450, 400, 350, 300, 250, 200, 150, 100, 80, 60, 50, 40, 35, 32, 30, or 25 Torr.

In one embodiment, the microparticles are stored at pressure for up to 1 week, 2 weeks, 3 weeks, 4 weeks, 1 month, 2 months, 3 months, 4 months, or more following the manufacture and isolation process. In one embodiment, the microparticles are stored for up to 1 week to up to 4 weeks at a pressure that is less than 700, 550, 500, 450, 400, 350, 300, 250, 200, 150, 100, 80, 60, 50, 40, 35, 32, or 30 Torr. In one embodiment, the microparticles are stored for up to 1 month to up to 2 months at a pressure that is less than 700, 550, 500, 450, 400, 350, 300, 250, 200, 150, 100, 80, 60, 50, 40, 35, 32, or 30 Torr. In one embodiment, the microparticles are stored for up to 3 months at a pressure that is less than 700, 550, 500, 450, 400, 350, 300, 250, 200, 150, 100, 80, 60, 50, 40, 35, 32, or 30 Torr

In one embodiment, the microparticles are stored at a temperature of between about 2-8° C. following the manufacturing and isolation process and the microparticles are vacuumed less than about 2 hours, 1 hour, 30 minutes, 15 minutes, or 10 minutes prior to in vivo injection. In one embodiment, the microparticles are stored at a temperature of between about 2-8° C. following the manufacturing and isolation process and the microparticles are vacuumed 1 hour to 30 minutes prior to in vivo injection. In one embodiment, the microparticles are stored at a temperature of between about 2-8° C. following the manufacturing and isolation process and the microparticles are vacuumed 30 minutes to 10 minutes prior to in vivo injection. In one embodiment, the microparticles are stored at a temperature of between about 2-8° C. following the manufacturing and isolation process and the microparticles are vacuumed immediately prior to in vivo injection.

In one embodiment, the microparticles are stored at a temperature of between about 2-8° C. and the microparticles are vacuumed for less than 1 hour, 30 minutes, 20 minutes, 15 minutes, or 10 minutes at a strength of less than about 35 Torr immediately prior to in vivo injection. In one embodiment, the microparticles are stored at a temperature of between about 2-8° C. and the microparticles are vacuumed for 1 hour to 30 minutes at a strength of less than about 35 Torr immediately prior to in vivo injection. In one embodiment, the microparticles are stored at a temperature of between about 2-8° C. and the microparticles are vacuumed for 30 minutes to 10 minutes at a strength of less than about 35 Torr immediately prior to in vivo injection. In one embodiment, the particles are suspended in a glass vial that is attached to a vial adapter and the vial adapter is in turn attached to a VacLok syringe (FIG. 21). A negative pressure is created in the vial by pulling the plunger of the syringe into a locking position as shown in FIG. 20C. In one embodiment, the vacuum treatment is conducted in a syringe of the 60 mL, 30 mL, 20 mL, or 10 mL size. The vacuum is then held in the syringe with the vial facing up and the large syringe attached for up to at least 10 minutes, 20 minutes, 30 minutes, 40 minutes, 50 minutes, 60 minutes, 70 minutes, 90 minutes, 100 minutes, or 129 minutes. The vacuum is released, the large syringe is detached, and a syringe is attached for in vivo injection.

In one embodiment, the particles are subjected to vacuum treatment at a strength of about 143 Torr for about at least 10 minutes, 20 minutes, 30 minutes, 40 minutes, 50 minutes, 60 minutes, 70 minutes, 80 minutes, 90 minutes, 100 minutes, or 120 minutes. In one embodiment, the particles are subjected to vacuum treatment at a strength of at least about 90, 89, 88, 87, 86, or 85 Torr for at least about at 10 minutes, 20 minutes, 30 minutes, or 40 minutes. In one embodiment, the particles are subjected to vacuum treatment at a strength of at least about 87 Torr for at least about 10 minutes, 20 minutes, 30 minutes, 40 minutes, 60 minutes, 90 minutes, or 120 minutes. In one embodiment, the particles are subjected to vacuum treatment at a strength of at least about 35, 34, 33, 32, 31, or 30 Torr for at least 5 minutes. In one embodiment, the particles are subjected to vacuum treatment at a strength of at least about 35, 34, 33, 32, 31, or 30 Torr for at least 8 minutes. In one embodiment, the particles are subjected to vacuum treatment at a strength of at least about 35, 34, 33, 32, 31, or 30 Torr for at least 10 minutes. In one embodiment, the particles are subjected to vacuum treatment at a strength of at least about 35, 34, 33, 32, 31, or 30 Torr for at least 20 minutes. In one embodiment, the particles are subjected to vacuum treatment at a strength of at least about 35, 34, 33, 32, 31, or 30 Torr for at least 30 minutes. In one embodiment, the particles are subjected to vacuum treatment at a strength of at least about 35, 34, 33, 32, 31, or 30 Torr for at least 40 minutes. In one embodiment, the particles are subjected to 30 Torr for at least 5, 10, 15, 20, 25, 30, 35, 40, 45, 50, 55, 60, 65, 70, 75, 80, 85, or 90 minutes. In one embodiment, the particles are subjected to vacuum treatment at a strength of about 35 Torr for at least 90 minutes. In one embodiment, the particles are subjected to vacuum treatment at a strength of about 35 Torr for at least 60 minutes. In one embodiment, the particles are subjected to vacuum treatment at a strength of about 35 Torr for at least 30 minutes. In one embodiment, the particles are subjected to vacuum treatment at a strength of about 35 Torr for at least 15 minutes. In one embodiment, the particles are subjected to vacuum treatment at a strength of about 35 Torr for at least 5 minutes. In one embodiment, the particles are subjected to vacuum treatment at a strength of about 32 Torr for at least 30 minutes. In one embodiment, the particles are subjected to vacuum treatment at a strength of about 32 Torr for at least 15 minutes. In one embodiment, the particles are subjected to vacuum treatment at a strength of about 32 Torr for at least 5 minutes. In one embodiment, the particles are subjected to vacuum treatment at a strength of about 30 Torr for at least 30 minutes. In one embodiment, the particles are subjected to vacuum treatment at a strength of about 30 Torr for at least 15 minutes. In one embodiment, the particles are subjected to vacuum treatment at a strength of about 30 Torr for at least 5 minutes.

In an alternative embodiment, the particles are suspended in a diluent in a vial attached to a vial adapter that is further attached to a 60 mL VacLok syringe containing a plunger (as shown in FIG. 21) wherein the plunger is pulled to the 50 mL mark and locked to create a negative pressure of approximately 30 Torr and the pressure is held for at least about 3, 5, 8, 10, 15, 20, 25, 30, or 35 minutes. In an alternative embodiment, the particles are suspended in a diluent in a vial attached to a vial adapter that is further attached to a 60 mL VacLok syringe containing a plunger wherein the plunger is pulled to the 45 mL mark, locked, and held for at least about 3, 5, 8, 10, 15, 20, 25, 30, 35, 40, 45, 50, 55, 60, 65, 70, 75, 80, 85, or 90 minutes. In an alternative embodiment, the particles are suspended in a diluent in a vial attached to a vial adapter that is further attached to a 60 mL VacLok syringe containing a plunger wherein the plunger is pulled to the 40 mL mark, locked, and the pressure is held for at least about 3, 5, 8, 10, 15, 20, 25, 30, or 35 minutes. In an alternative embodiment, the particles are suspended in a diluent in a vial attached to a vial adapter that is further attached to a 60 mL VacLok syringe containing a plunger wherein the plunger is pulled to the 35 mL mark, locked, and held for about at least 3, 5, 8, 10, 15, 20, 25, 30, or 35 minutes. In an alternative embodiment, the particles are suspended in a diluent in a vial attached to a vial adapter that is further attached to a 60 mL VacLok syringe containing a plunger wherein the plunger is pulled to the 30 mL mark, locked, and held for at least about 3, 5, 8, 10, 15, 20, 25, 30, or 35 minutes. In an alternative embodiment, the particles are suspended in a diluent in a vial attached to a vial adapter that is further attached to a 60 mL VacLok syringe containing a plunger wherein the plunger is pulled to the 25 mL mark, locked, and held for at least about 3, 5, 8, 10, 15, 20, 25, 30, or 35 minutes.

In certain embodiments, the particles are suspended in a diluent and the suspension is exposed to a pressure of less than 40 Torr for between about 90 minutes and 1 minute, between about 60 minutes and 1 minute, between about 45 minutes and 1 minute, between about 30 minutes and 1 minute, between about 15 minutes and 1 minute, or between about 5 minutes and 1 minute.

In certain embodiments, the particles are suspended in a diluent and the suspension is exposed to a pressure of less than 30 Torr for between about 90 minutes and 1 minute, between about 60 minutes and 1 minute, between about 45 minutes and 1 minute, between about 30 minutes and 1 minute, between about 15 minutes and 1 minute, or between about 5 minutes and 1 minute.

In one embodiment, the microparticles are suspended in a diluent of 10× ProVisc-diluted (0.1% HA in PBS) solution. In one embodiment, the microparticles are suspended in a diluent of 20×-diluted ProVisc (0.05% HA in PBS). In one embodiment, the microparticles are suspended in a diluent of 40×-diluted ProVisc (0.025% HA in PBS).

In one embodiment, the particles are suspended in the diluent at a concentration of 100 mg/mL, 150 mg/mL, 200 mg/mL, 250 mg/mL, 300 mg/mL, 350 mg/mL, 400 mg/mL, 450 mg/mL or 500 mg/mL. In one embodiment, the particles are suspended in 10×-diluted ProVisc (0.1% HA in PBS) solution and the suspension has a final concentration of 200 mg/mL. In one embodiment, the particles are suspended in 10×-diluted ProVisc (0.1% HA in PBS) solution and the suspension has a final concentration of 400 mg/mL. In one embodiment, the particles are suspended in a 20×-diluted ProVisc (0.05% HA in PBS) and the suspension has a final concentration of 200 mg/mL. In one embodiment, the particles are suspended in a 20×-diluted ProVisc (0.05% HA in PBS) and the suspension has a final concentration of 400 mg/mL. In one embodiment, the particles are suspended in a 40×-diluted ProVisc (0.025% HA in PBS) and the suspension has a concentration of 200 mg/mL. In one embodiment, the particles are suspended in a 40×-diluted ProVisc (0.025% HA in PBS) and the suspension has a concentration of 400 mg/mL.

### The Addition of an Excipient

In one embodiment, the process for preparing an improved microparticle suspension prior to injection is the addition of at least one excipient, typically prior to lyophilization that reduces the amount of air adhering to the particles. Particles are suspended in an aqueous solution and sonicated before being flash frozen in −80° C. ethanol and lyophilized overnight. In one embodiment, the particles are suspended in an aqueous sugar solution that is 1%, 2%, 3%, 4%, 5%, 6%, 7%, 8%, 9%, 10%, 11%, 12%, 13%, 14%, or 15% sugar. In one embodiment, the sugar is sucrose. In one embodiment, the sugar is mannitol. In one embodiment, the sugar is trehalose. In one embodiment, the sugar is glucose. In one embodiment, the sugar is selected from arabinose, fucose, mannose, rhamnose, xylose, D-xylose, glucose, fructose, ribose, D-ribose, galactose, dextrose, dextran, lactose, maltodextrin, maltose, glycerol, erythritol, threitol, arabitol, xylitol, ribitol, sorbitol, galactitol, fucitol, iditol, inositol, volemitol, isomalt, maltitol, lactitol, maltotriitol, maltotetraitol, and polyglycitol. In an alternative embodiment, the sugar is selected from aspartame, saccharin, stevia, sucralose, acesulfame potassium, advantame, alitame, neotame, and sucralose. In one embodiment, the particles are suspended in an aqueous sugar solution that is 1%, 2%, 3%, 4%, 5%, 6%, 7%, 8%, 9%, 10%, 11%, 12%, 13%, 14%, or 15% sucrose. In one embodiment, the particles are suspended in a 1% sucrose solution. In one embodiment, the particles are suspended in a 10% sucrose solution. In one embodiment, the particles are suspended in an aqueous sugar solution that is 1%, 2%, 3%, 4%, 5%, 6%, 7%, 8%, 9%, 10%, 11%, 12%, 13%, 14%, or 15% mannitol. In one embodiment, the particles are suspended in a 1% mannitol solution. In one embodiment, the particles are suspended in a 10% mannitol solution. In one embodiment, the particles are suspended in a 1% trehalose solution. In one embodiment, the particles are suspended in a 10% trehalose solution. In one embodiment, the particles are suspended in an aqueous sugar solution that is 1%, 2%, 3%, 4%, 5%, 6%, 7%, 8%, 9%, 10%, 11%, 12%, 13%, 14%, or 15% trehalose. In an alternative embodiment, the particles are suspended in a small surfactant molecule, including, but not limited to tween 20 or tween 80. In an alternative embodiment, the particles are flash frozen in −80° C. methanol or isopropanol.

### Sonication

In one embodiment, a process for providing an improved microparticle suspension prior to injection is sonication wherein particles are suspended in a diluent and the suspension of microparticles is sonicated for at least 30 minutes, at least 25 minutes, at least 20 minutes, at least 15 minutes, at least 10 minutes, at least 8 minutes, at least 5 minutes, or at least 3 minutes. In one embodiment, the particle solutions are sonicated at a frequency of 40 kHz. In one embodiment, the particles are suspended in the diluent at a concentration of 100 mg/mL, 150 mg/mL, 200 mg/mL, 250 mg/mL, 300 mg/mL, 350 mg/mL, 400 mg/mL, 450 mg/mL or 500 mg/mL. In one embodiment, the diluent is hyaluronic acid. In an alternative embodiment, the diluent is selected from hyaluronic acid, hydroxypropyl methylcellulose, chondroitin sulfate, or a blend of at least two diluents selected from hyaluronic acid, hydroxypropyl methylcellulose, and chondroitin sulfate. In an alternative embodiment, the diluent is selected from aacia, tragacanth, alginic acid, carrageenan, locust bean gum, gellan gum, guar gum, gelatin, starch, methylcellulose, sodium carboxymethylcellulose, hydroxyethylcellulose, hydroxypropyl cellulose, Carbopol® homopolymers (acrylic acid crosslinked with allyl sucrose or allyl pentaerythritol), and Carbopol® copolymers (acrylic acid and C₁₀-C₃₀ alkyl acrylate crosslinked with allyl pentaerythritol).

In certain embodiments, a combination of vacuum treatment, the addition of excipients, and sonication can be used following isolation and reconstitution of the microparticles. In certain embodiments, the methods for enhancing wettability are conducted at least 1 hour prior to in vivo injection, at least 45 minutes prior to in vivo injection, at least 30 minutes prior to in vivo injection, at least 25 minutes prior to in vivo injection, at least 20 minutes prior to injection, at least 15 minutes prior to in vivo injection, at least 10 minutes prior to in vivo injection, or at least 5 minutes prior to in vivo injection. In one embodiment, the vacuum treatment, addition of an excipient, and/or sonication is conducted immediately before in vivo injection. In one embodiment, the particles are vacuumed at a strength of less than 35 Torr for less than 30 minutes and are immediately injected in vivo. In an alternative embodiment, the particles are vacuumed at a strength of less than 35 Torr for less than 20 minutes and are immediately injected in vivo. In an alternative embodiment, the particles are vacuumed at a strength of less than 35 Torr for less than 15 minutes and are immediately injected in vivo. In an alternative embodiment, the particles are vacuumed at a strength of less than 35 Torr for less than 10 minutes and are immediately injected in vivo.

In one embodiment, the microparticles are stored at a temperature of between about 2-8° C. following the manufacturing and isolation process and the microparticles are held under negative pressure for about 24, 12, 8, 6, 2 hours, 1 hour, 30 minutes, 15 minutes, or 10 minutes or less prior to in vivo injection. In one embodiment, the microparticles are stored at a temperature of between about 2-8° C. following the manufacturing and isolation process and the microparticles are held under negative pressure 1 hour to 30 minutes prior to in vivo injection. In one embodiment, the microparticles are stored at a temperature of between about 2-8° C. following the manufacturing and isolation process and the microparticles are vacuumed 30 minutes to 10 minutes prior to in vivo injection. In one embodiment, the microparticles are stored at a temperature of between about 2-8° C. following the manufacturing and isolation process and the microparticles are vacuumed immediately prior to in vivo injection.

In one embodiment, the microparticles are stored at a temperature of between about 2-8° C. and the microparticles are vacuumed for less than 1 hour, 30 minutes, 20 minutes, 15 minutes, or 10 minutes at a strength of less than about 35 Torr immediately prior to in vivo injection. In one embodiment, the microparticles are stored at a temperature of between about 2-8° C. and the microparticles are vacuumed for 1 hour to 30 minutes at a strength of less than about 35 Torr immediately prior to in vivo injection. In one embodiment, the microparticles are stored at a temperature of between about 2-8° C. and the microparticles are vacuumed for 30 minutes to 10 minutes at a strength of less than about 35 Torr immediately prior to in vivo injection.

In one embodiment, the microparticles are stored at negative pressure for up to 1 week, 2 weeks, 3 weeks, 4 weeks, 1 month, 2 months, 3 months, 4 months, or more following the manufacture and isolation process. In one embodiment, the microparticles are stored for up to 1 week to up to 4 weeks at a negative pressure that is less than 700, 550, 500, 450, 400, 350, 300, 250, 200, 150, 100, 80, 60, 50, 40, 35, 32, or 30 Torr. In one embodiment, the microparticles are stored for up to 1 month to up to 2 months at a negative pressure that is less than 700, 550, 500, 450, 400, 350, 300, 250, 200, 150, 100, 80, 60, 50, 40, 35, 32, or 30 Torr. In one embodiment, the microparticles are stored for up to 3 months at a negative pressure that is less than 700, 550, 500, 450, 400, 350, 300, 250, 200, 150, 100, 80, 60, 50, 40, 35, 32, or 30 Torr.

Thus, microparticles and microparticle suspensions are provided that have improved aggregation to a pellet for medical therapy due to enhanced wettability in vivo. Examples of processes that provide improved aggregation of particles to the desired ocular pellet include, but are not limited to, one or a combination of 1) applying a vacuum to the particle suspension to facilitate the disassociation of air from particles; 2) adding one or more excipients to reduce surface hydrophobicity of particles and thus reduce the amount of air adhering to the particles; and, 3) sonication to facilitate the disassociation of air from the particles, either prior to lyophilization or other drying means to make a solid reconstitutable microparticle material, or by carrying out one or more of these processes after reconstitution.

These processes can be used at the time the particles are being prepared to produce the powder or material that is stored and then later resuspended (for example, prior to lyophilization) for injection. In one example, the vessel with the dried microparticles can be placed under pressure for storage before use. In another non-limiting example, the container storing the surface-treated microparticles can be placed under vacuum directly before administration. In other embodiments, it is not necessary to remove air or gas from the active-loaded microparticle at any stage of manufacture to achieve a suitable therapeutic effect.

In one embodiment, surface-modified solid aggregating microparticles that include at least one biodegradable polymer, wherein the surface-modified solid aggregating microparticles have a solid core, include a therapeutic agent selected from a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV, have a modified surface which has been treated under mild conditions at a temperature at or less than about 18° C. to remove surface surfactant, are sufficiently small to be injected in vivo, have been treated to remove or decrease air or gas adhered on the microparticle, and are capable of aggregating in vivo to form at least one pellet of at least 500 μm in vivo to provide sustained drug delivery in vivo for at least one month, two months, three months, four months, five months, six months or seven months or more are provided.

In one embodiment, surface-modified solid aggregating microparticles that include at least one biodegradable polymer, wherein the surface-modified solid aggregating microparticles have a solid core, include a therapeutic agent selected from Compound 1-1, Compound 2-1, Compound 3-1, Compound 16-2, Compound 25-1, or Compound 26-1, have a modified surface which has been treated under mild conditions at a temperature at or less than about 18° C. to remove surface surfactant, are sufficiently small to be injected in vivo, have been treated to remove or decrease air or gas adhered on the microparticle, and are capable of aggregating in vivo to form at least one pellet of at least 500 μm in vivo to provide sustained drug delivery in vivo for at least one month, two months, three months, four months, five months, six months or seven months or more are provided.

Common techniques for preparing particles include, but are not limited to, solvent evaporation, solvent removal, spray drying, phase inversion, coacervation, and low temperature casting. Suitable methods of particle formulation are briefly described below. Pharmaceutically acceptable excipients, including pH modifying agents, disintegrants, preservatives, and antioxidants, can optionally be incorporated into the particles during particle formation.

### Solvent Evaporation

In this method, the drug (or polymer matrix and one or more Drugs) is dissolved in a volatile organic solvent, such as methylene chloride. The organic solution containing the drug is then suspended in an aqueous solution that contains a surface active agent such as poly(vinyl alcohol). The resulting emulsion is stirred until most of the organic solvent evaporated, leaving solid nanoparticles. The resulting nanoparticles are washed with water and dried overnight in a lyophilizer. Nanoparticles with different sizes and morphologies can be obtained by this method.

Drugs which contain labile polymers, such as certain polyanhydrides, may degrade during the fabrication process due to the presence of water. For these polymers, the following two methods, which are performed in completely anhydrous organic solvents, can be used.

### Solvent Removal

Solvent removal can also be used to prepare particles from drugs that are hydrolytically unstable. In this method, the drug (or polymer matrix and one or more Drugs) is dispersed or dissolved in a volatile organic solvent such as methylene chloride. This mixture is then suspended by stirring in an organic oil (such as silicon oil) to form an emulsion. Solid particles form from the emulsion, which can subsequently be isolated from the supernatant. The external morphology of spheres produced with this technique is highly dependent on the identity of the drug.

In one embodiment a compound of the present invention is administered to a patient in need thereof as particles formed by solvent removal. In another embodiment the present invention provides particles formed by solvent removal comprising a compound of the present invention and one or more pharmaceutically acceptable excipients as defined herein. In another embodiment the particles formed by solvent removal comprise a compound of the present invention and an additional therapeutic agent. In a further embodiment the particles formed by solvent removal comprise a compound of the present invention, an additional therapeutic agent, and one or more pharmaceutically acceptable excipients. In another embodiment any of the described particles formed by solvent removal can be formulated into a tablet and then coated to form a coated tablet. In an alternative embodiment the particles formed by solvent removal are formulated into a tablet but the tablet is uncoated.

### Spray Drying

In this method, the drug (or polymer matrix and one or more Drugs) is dissolved in an organic solvent such as methylene chloride. The solution is pumped through a micronizing nozzle driven by a flow of compressed gas, and the resulting aerosol is suspended in a heated cyclone of air, allowing the solvent to evaporate from the micro droplets, forming particles. Particles ranging between 0.1-10 microns can be obtained using this method.

In one embodiment a compound of the present invention is administered to a patient in need thereof as a spray dried dispersion (SDD). In another embodiment the present invention provides a spray dried dispersion (SDD) comprising a compound of the present invention and one or more pharmaceutically acceptable excipients as defined herein. In another embodiment the SDD comprises a compound of the present invention and an additional therapeutic agent. In a further embodiment the SDD comprises a compound of the present invention, an additional therapeutic agent, and one or more pharmaceutically acceptable excipients. In another embodiment any of the described spray dried dispersions can be coated to form a coated tablet. In an alternative embodiment the spray dried dispersion is formulated into a tablet but is uncoated.

### Phase Inversion

Particles can be formed from drugs using a phase inversion method. In this method, the drug (or polymer matrix and one or more Drugs) is dissolved in a “good” solvent, and the solution is poured into a strong non solvent for the drug to spontaneously produce, under favorable conditions, microparticles or nanoparticles. The method can be used to produce nanoparticles in a wide range of sizes, including, for example, about 100 nanometers to about 10 microns, typically possessing a narrow particle size distribution.

In one embodiment a compound of the present invention is administered to a patient in need thereof as particles formed by phase inversion. In another embodiment the present invention provides particles formed by phase inversion comprising a compound of the present invention and one or more pharmaceutically acceptable excipients as defined herein. In another embodiment the particles formed by phase inversion comprise a compound of the present invention and an additional therapeutic agent. In a further embodiment the particles formed by phase inversion comprise a compound of the present invention, an additional therapeutic agent, and one or more pharmaceutically acceptable excipients. In another embodiment any of the described particles formed by phase inversion can be formulated into a tablet and then coated to form a coated tablet. In an alternative embodiment the particles formed by phase inversion are formulated into a tablet but the tablet is uncoated.

### Coacervation

Techniques for particle formation using coacervation are known in the art, for example, in GB-B-929 406; GB-B-929 40 1; and U.S. Pat. Nos. 3,266,987, 4,794,000, and 4,460,563. Coacervation involves the separation of a drug (or polymer matrix and one or more Drugs)solution into two immiscible liquid phases. One phase is a dense coacervate phase, which contains a high concentration of the drug, while the second phase contains a low concentration of the drug. Within the dense coacervate phase, the drug forms nanoscale or microscale droplets, which harden into particles. Coacervation may be induced by a temperature change, addition of a non-solvent or addition of a micro-salt (simple coacervation), or by the addition of another polymer thereby forming an interpolymer complex (complex coacervation).

In one embodiment a compound of the present invention is administered to a patient in need thereof as particles formed by coacervation. In another embodiment the present invention provides particles formed by coacervation comprising a compound of the present invention and one or more pharmaceutically acceptable excipients as defined herein. In another embodiment the particles formed by coacervation comprise a compound of the present invention and an additional therapeutic agent. In a further embodiment the particles formed by coacervation comprise a compound of the present invention, an additional therapeutic agent, and one or more pharmaceutically acceptable excipients. In another embodiment any of the described particles formed by coacervation can be formulated into a tablet and then coated to form a coated tablet. In an alternative embodiment the particles formed by coacervation are formulated into a tablet but the tablet is uncoated.

### Low Temperature Casting

Methods for very low temperature casting of controlled release microspheres are described in U.S. Pat. No. 5,019,400 to Gombotz et al. In this method, the drug (or polymer matrix and

Sunitinib) is dissolved in a solvent. The mixture is then atomized into a vessel containing a liquid non-solvent at a temperature below the freezing point of the drug solution which freezes the drug droplets. As the droplets and non-solvent for the drug are warmed, the solvent in the droplets thaws and is extracted into the non-solvent, hardening the microspheres.

In one embodiment a compound of the present invention is administered to a patient in need thereof as particles formed by low temperature casting. In another embodiment the present invention provides particles formed by low temperature casting comprising a compound of the present invention and one or more pharmaceutically acceptable excipients as defined herein. In another embodiment the particles formed by low temperature casting comprise a compound of the present invention and an additional therapeutic agent. In a further embodiment the particles formed by low temperature casting comprise a compound of the present invention, an additional therapeutic agent, and one or more pharmaceutically acceptable excipients. In another embodiment any of the described particles formed by low temperature casting can be formulated into a tablet and then coated to form a coated tablet. In an alternative embodiment the particles formed by low temperature casting are formulated into a tablet but the tablet is uncoated.

III. Controlled Release of Therapeutic Agent

The rate of release of the therapeutic agent can be related to the concentration of therapeutic agent dissolved in polymeric material. In many embodiments, the polymeric composition includes non-therapeutic agents that are selected to provide a desired solubility of the therapeutic agent. The selection of polymer can be made to provide the desired solubility of the therapeutic agent in the matrix, for example, a hydrogel may promote solubility of hydrophilic material. In some embodiments, functional groups can be added to the polymer to increase the desired solubility of the therapeutic agent in the matrix. In some embodiments, additives may be used to control the release kinetics of therapeutic agent, for example, the additives may be used to control the concentration of therapeutic agent by increasing or decreasing solubility of the therapeutic agent in the polymer so as to control the release kinetics of the therapeutic agent. The solubility may be controlled by including appropriate molecules and/or substances that increase and/or decrease the solubility of the dissolved from of the therapeutic agent to the matrix. The solubility of the therapeutic agent may be related to the hydrophobic and/or hydrophilic properties of the matrix and therapeutic agent. Oils and hydrophobic molecules and can be added to the polymer to increase the solubility of hydrophobic treatment agent in the matrix.

Instead of or in addition to controlling the rate of migration based on the concentration of therapeutic agent dissolved in the matrix, the surface area of the polymeric composition can be controlled to attain the desired rate of drug migration out of the composition. For example, a larger exposed surface area will increase the rate of migration of the active agent to the surface, and a smaller exposed surface area will decrease the rate of migration of the active agent to the surface. The exposed surface area can be increased in any number of ways, for example, by any of castellation of the exposed surface, a porous surface having exposed channels connected with the tear or tear film, indentation of the exposed surface, protrusion of the exposed surface. The exposed surface can be made porous by the addition of salts that dissolve and leave a porous cavity once the salt dissolves. In the present invention, these trends can be used to decrease the release rate of the active material from the polymeric composition by avoiding these paths to quicker release. For example, the surface area can be minimized, or channels avoided.

Further, an implant may be used that includes the ability to release two or more drugs in combination, for example, the structure disclosed in U.S. Pat. No. 4,281,654 (Shell), for example, in the case of glaucoma treatment, it may be desirable to treat a patient with multiple prostaglandins or a prostaglandin and a cholinergic agent or an adrenergic antagonist (beta blocker), for example, Alphagan (Allegan, Irvine, Calif., USA), or a prostaglandin and a carbonic anhydrase inhibitor.

In addition, drug impregnated meshes may be used, for example, those disclosed in U.S. Patent Application Publication No. 2002/0055701 or layering of biostable polymers as described in U.S. Patent Application Publication No. 2005/0129731. Certain polymer processes may be used to incorporate drug into the devices, as described herein, for example, so-called “self-delivering drugs” or Polymer Drugs (Polymerix Corporation, Piscataway, N.J., USA) are designed to degrade only into therapeutically useful compounds and physiologically inert linker molecules, further detailed in U.S. Patent Application Publication No. 2005/0048121 (East), hereby incorporated by reference in its entirety. Such delivery polymers may be employed in the devices, as described herein, to provide a release rate that is equal to the rate of polymer erosion and degradation and is constant throughout the course of therapy. Such delivery polymers may be used as device coatings or in the form of microspheres for a drug depot injectable (for example, a reservoir described herein). A further polymer delivery technology may also be adapted to the devices, as described herein, for example, that described in U.S. Patent Application Publication No. 2004/0170685 (Carpenter), and technologies available from Medivas (San Diego, Calif., USA).

In another embodiment any of the above delivery systems can be used to facilitate or enhance delivery through mucus.

IV. Method of Treatment

In one embodiment, a compound of Formula I, Formula II, Formula III, Formula IV, Formula V, Formula VI, Formula VII, Formula VIII, Formula IX, Formula X, Formula XI, Formula XII, Formula XIII, Formula IV, or Formula V as described herein, or a pharmaceutically acceptable salt thereof is administered to treat or prevent a disorder related to an ocular disorder such as glaucoma, a disorder mediated by carbonic anhydrase, a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD), neovascular age-related macular degeneration (NVAMD), geographic atrophy or diabetic retinopathy.

Non-limiting exemplary eye disorders or diseases treatable with the composition includes age related macular degeneration, alkaline erosive keratoconjunctivitis, allergic conjunctivitis, allergic keratitis, anterior uveitis, Behcet's disease, blepharitis, blood-aqueous barrier disruption, chorioiditis, chronic uveitis, conjunctivitis, contact lens-induced keratoconjunctivitis, corneal abrasion, corneal trauma, corneal ulcer, crystalline retinopathy, cystoid macular edema, dacryocystitis, diabetic keratophathy, diabetic macular edema, diabetic retinopathy, dry eye disease, dry age-related macular degeneration, geographic atrophy, eosinophilic granuloma, episcleritis, exudative macular edema, Fuchs' Dystrophy, giant cell arteritis, giant papillary conjunctivitis, glaucoma, glaucoma surgery failure, graft rejection, herpes zoster, inflammation after cataract surgery, iridocorneal endothelial syndrome, iritis, keratoconjunctiva sicca, keratoconjunctival inflammatory disease, keratoconus, lattice dystrophy, map-dot-fingerprint dystrophy, necrotic keratitis, neovascular diseases involving the retina, uveal tract or cornea, for example, neovascular glaucoma, corneal neovascularization, neovascularization resulting following a combined vitrectomy and lensectomy, neovascularization of the optic nerve, and neovascularization due to penetration of the eye or contusive ocular injury, neuroparalytic keratitis, non-infectious uveitisocular herpes, ocular lymphoma, ocular rosacea, ophthalmic infections, ophthalmic pemphigoid, optic neuritis, panuveitis, papillitis, pars planitis, persistent macular edema, phacoanaphylaxis, posterior uveitis, post-operative inflammation, proliferative diabetic retinopathy, proliferative sickle cell retinopathy, proliferative vitreoretinopathy, retinal artery occlusion, retinal detachment, retinal vein occlusion, retinitis pigmentosa, retinopathy of prematurity, rubeosis iritis, scleritis, Stevens-Johnson syndrome, sympathetic ophthalmia, temporal arteritis, thyroid associated ophthalmopathy, uveitis, vernal conjunctivitis, vitamin A insufficiency-induced keratomalacia, vitreitis, and wet age-related macular degeneration.

Any of the compounds described herein (Formula I, Formula II, Formula III, Formula IV, Formula V, Formula VI, Formula VII, Formula VIII, Formula IX, Formula X, Formula XI, Formula XII, Formula XIII, Formula IV, or Formula V or a pharmaceutically acceptable salt thereof) can be administered to the eye in a composition as described herein in any desired form of administration, including via intravitreal, intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, and tear duct injections, or through a mucus, mucin, or a mucosal barrier, in an immediate or controlled release fashion.

In an alternative embodiment, any of the compounds or pharmaceutically acceptable salts or compositions thereof can be administered systemically, topically, parentally, intravenously, subcutaneously, intramuscularly, transdermally, buccally, or sublingually in an effective amount.

In an alternative embodiment, any of the compounds or pharmaceutically acceptable salts or compositions thereof can be administered systemically for the inhibition of tumor/cancer cell growth or cell proliferation in tumor/cancer cells. The treatment of cellular proliferative disorders, includes solid tumors and non-solid tumors, for example, leukemia. Non-limiting examples of cancer include hematological malignancies, oral carcinomas (for example of the lip, tongue or pharynx), digestive organs (for example esophagus, stomach, small intestine, colon, large intestine, or rectum), liver and biliary passages, pancreas, respiratory system such as larynx or lung (small cell and non-small cell), bone, connective tissue, skin (e.g., melanoma), breast, reproductive organs (uterus, cervix, testicles, ovary, or prostate), urinary tract (e.g., bladder or kidney), brain and endocrine glands such as the thyroid.

### EXAMPLES

### General Methods

All nonaqueous reactions were performed under an atmosphere of dry argon or nitrogen gas using anhydrous solvents. The progress of reactions and the purity of target compounds were determined using one of the two liquid chromatography (LC) methods listed below. The structure of starting materials, intermediates, and final products was confirmed by standard analytical techniques, including NMR spectroscopy and mass spectrometry.

The compounds described herein can be prepared by methods known by those skilled in the art. In one non-limiting example the disclosed compounds can be made by the schemes below.

### Example 1. Non-Limiting Examples of Compounds of Formula I

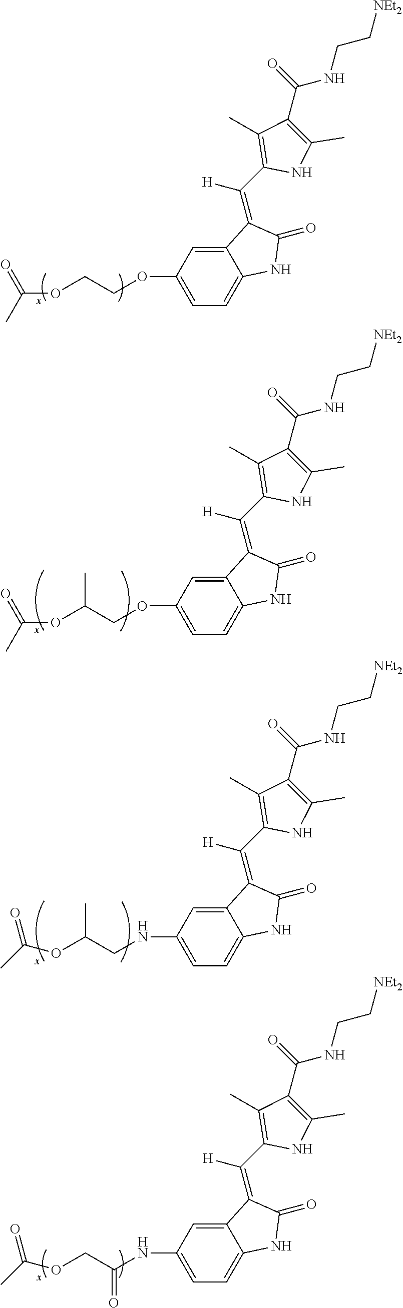

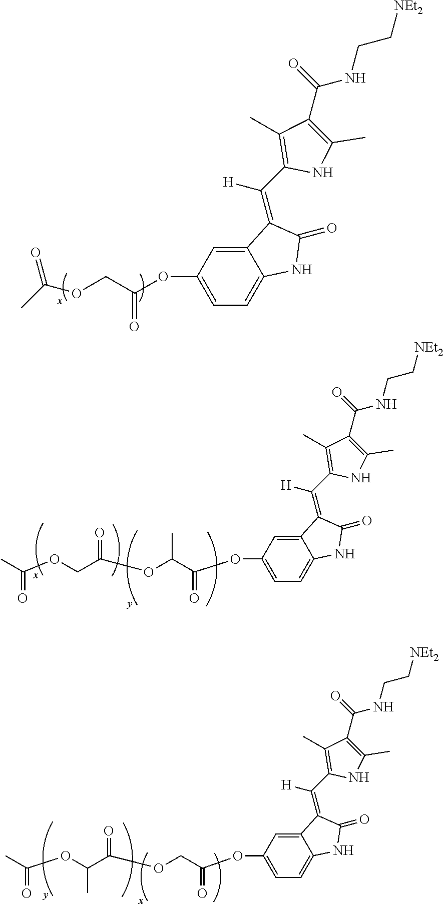

In one embodiment, x is independently an integer between 1 and 12 (1, 2, 3, 4, 5, 6, 7, 8, 9, 10, 11, or 12).

In one embodiment, x is independently an integer between 1 and 10 (1, 2, 3, 4, 5, 6, 7, 8, 9, or 10).

In one embodiment, x is independently an integer between 1 and 8 (1, 2, 3, 4, 5, 6, 7, or 8).

In one embodiment, x is independently an integer between 1 and 6 (1, 2, 3, 4, 5, or 6).

In one embodiment, x is independently an integer between 4 and 10 (4, 5, 6, 7, 8, 9, or 10).

In one embodiment, x is 1.

In one embodiment, x is 2.

In one embodiment, x is 3.

In one embodiment, x is 4.

In one embodiment, x is 6.

In one embodiment, x is 8.

In one embodiment, x is 10.

### Example 2. Non-Limiting Examples of Compounds of Formula II and Formula III

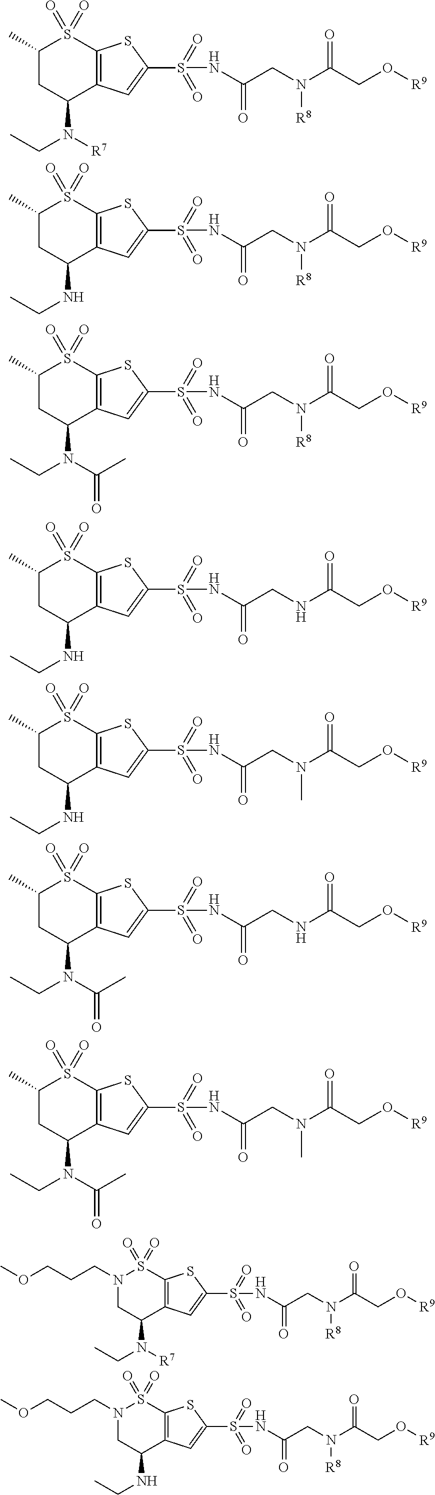

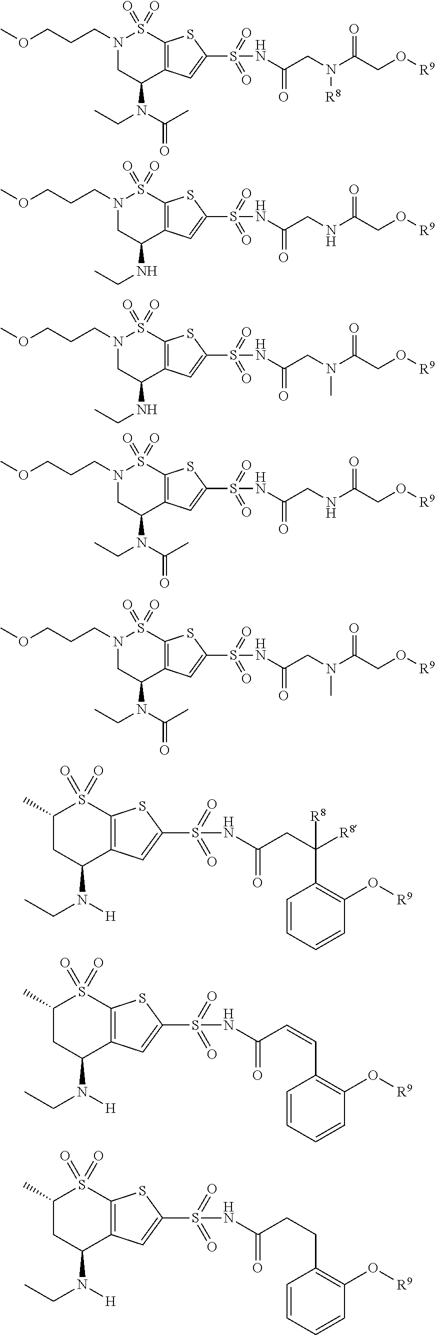

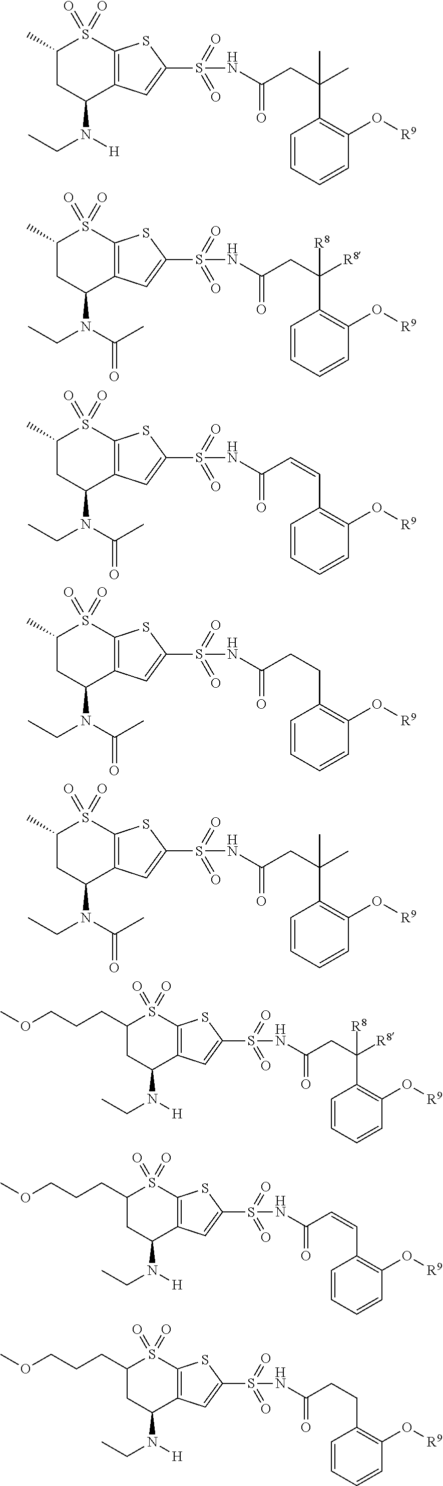

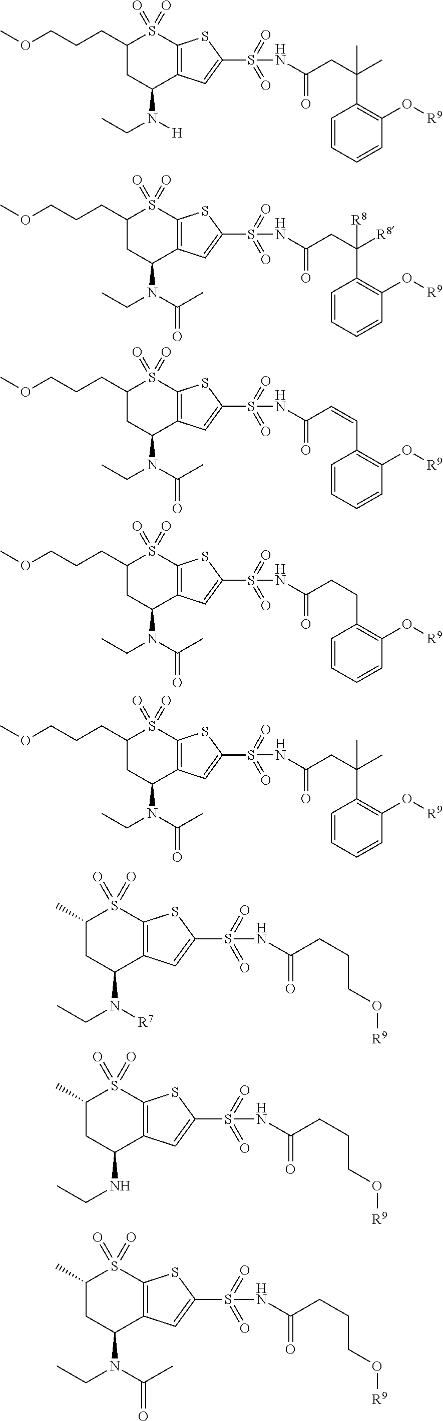

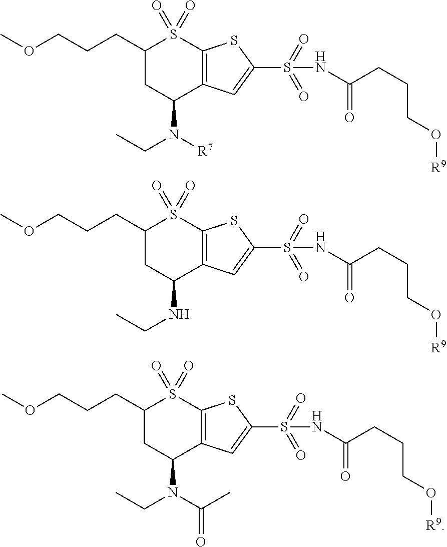

In one embodiment R⁹ is

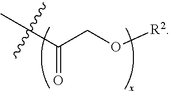

In one embodiment R⁹ is

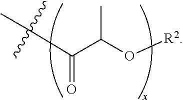

In one embodiment R⁹ is

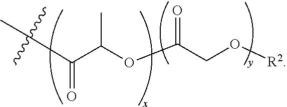

In one embodiment R⁹ is

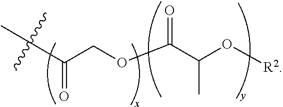

In one embodiment x is 1 and y is 1.

In one embodiment x is 1 and y is 2.

In one embodiment x is 1 and y is 3.

In one embodiment x is 1 and y is 4.

In one embodiment x is 1 and y is 5.

In one embodiment x is 1 and y is 6.

In one embodiment x is 1 and y is 7.

In one embodiment x is 1 and y is 8.

In one embodiment x is 2 and y is 1.

In one embodiment x is 2 and y is 2.

In one embodiment x is 2 and y is 3.

In one embodiment x is 2 and y is 4.

In one embodiment x is 2 and y is 5.

In one embodiment x is 2 and y is 6.

In one embodiment x is 2 and y is 7.

In one embodiment x is 2 and y is 8.

In one embodiment x is 3 and y is 1.

In one embodiment x is 3 and y is 2.

In one embodiment x is 3 and y is 3.

In one embodiment x is 3 and y is 4.

In one embodiment x is 3 and y is 5.

In one embodiment x is 3 and y is 6.

In one embodiment x is 3 and y is 7.

In one embodiment x is 3 and y is 8.

In one embodiment x is 4 and y is 1.

In one embodiment x is 4 and y is 2.

In one embodiment x is 4 and y is 3.

In one embodiment x is 4 and y is 4.

In one embodiment x is 4 and y is 5.

In one embodiment x is 4 and y is 6.

In one embodiment x is 4 and y is 7.

In one embodiment x is 4 and y is 8.

In one embodiment x is 5 and y is 1.

In one embodiment x is 5 and y is 2.

In one embodiment x is 5 and y is 3.

In one embodiment x is 5 and y is 4.

In one embodiment x is 5 and y is 5.

In one embodiment x is 5 and y is 6.

In one embodiment x is 5 and y is 7.

In one embodiment x is 5 and y is 8.

In one embodiment x is 6 and y is 1.

In one embodiment x is 6 and y is 2.

In one embodiment x is 6 and y is 3.

In one embodiment x is 6 and y is 4.

In one embodiment x is 6 and y is 5.

In one embodiment x is 6 and y is 6.

In one embodiment x is 6 and y is 7.

In one embodiment x is 6 and y is 8.

In one embodiment x is 7 and y is 1.

In one embodiment x is 7 and y is 2.

In one embodiment x is 7 and y is 3.

In one embodiment x is 7 and y is 4.

In one embodiment x is 7 and y is 5.

In one embodiment x is 7 and y is 6.

In one embodiment x is 7 and y is 7.

In one embodiment x is 7 and y is 8.

In one embodiment x is 8 and y is 1.

In one embodiment x is 8 and y is 2.

In one embodiment x is 8 and y is 3.

In one embodiment x is 8 and y is 4.

In one embodiment x is 8 and y is 5.

In one embodiment x is 8 and y is 6.

In one embodiment x is 8 and y is 7.

In one embodiment x is 8 and y is 8.

### Example 3. Non-Limiting Examples of Compounds of Formula IV and Formula V

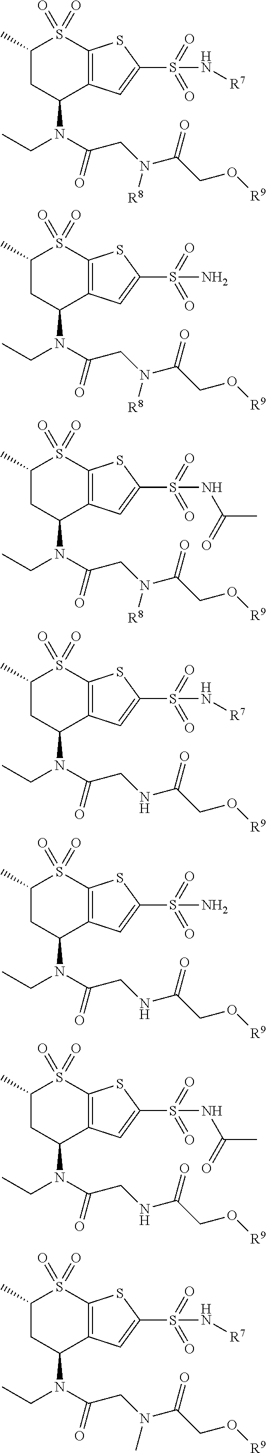

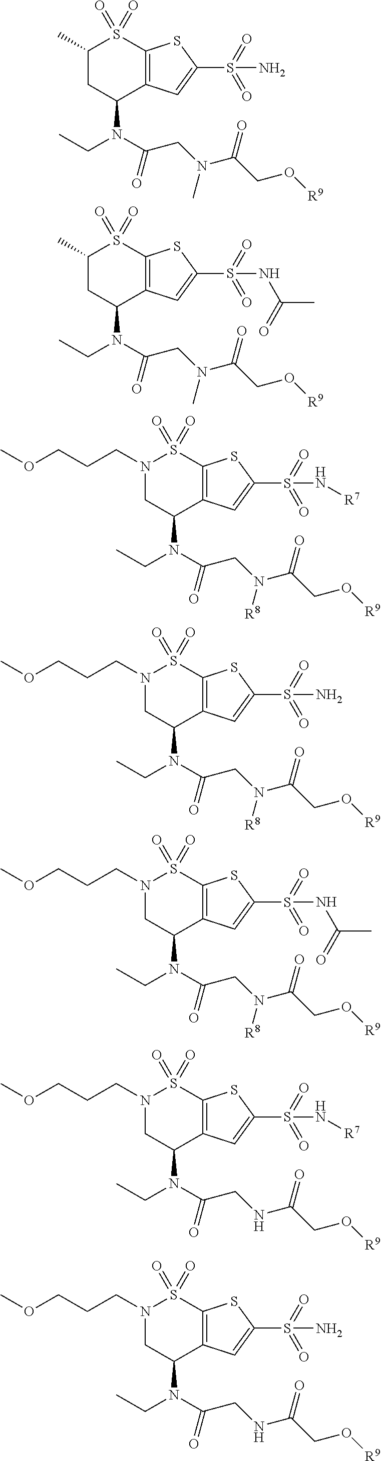

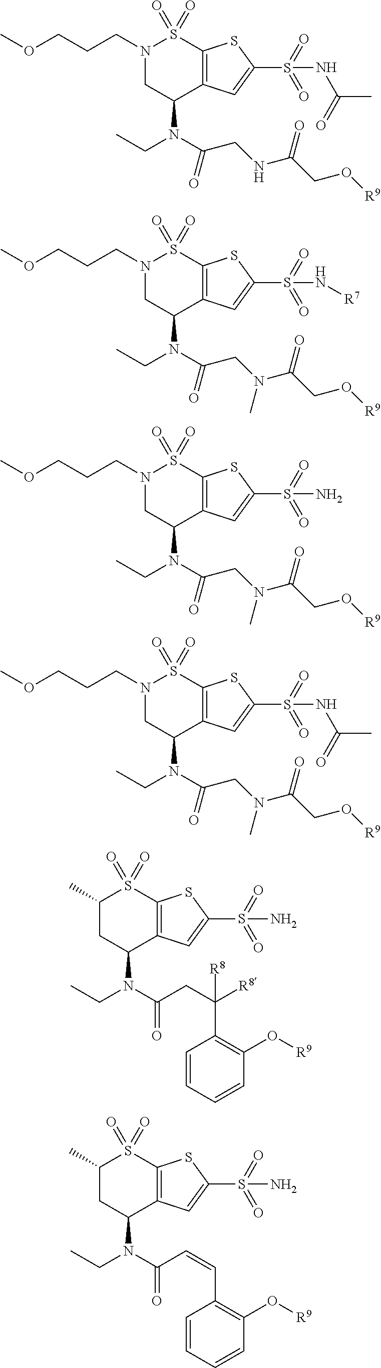

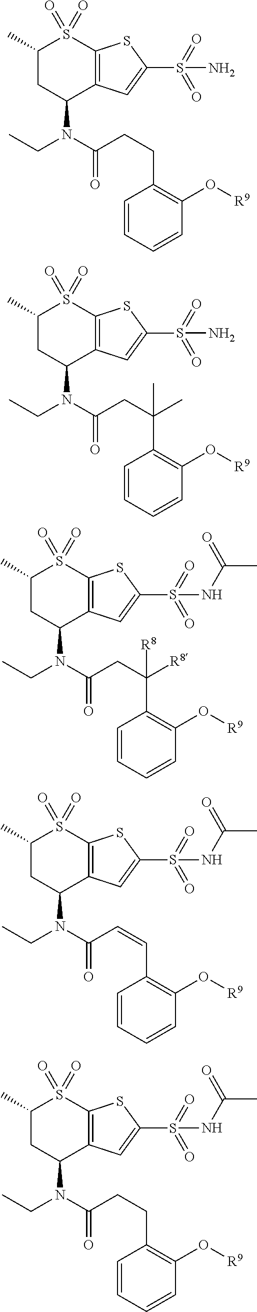

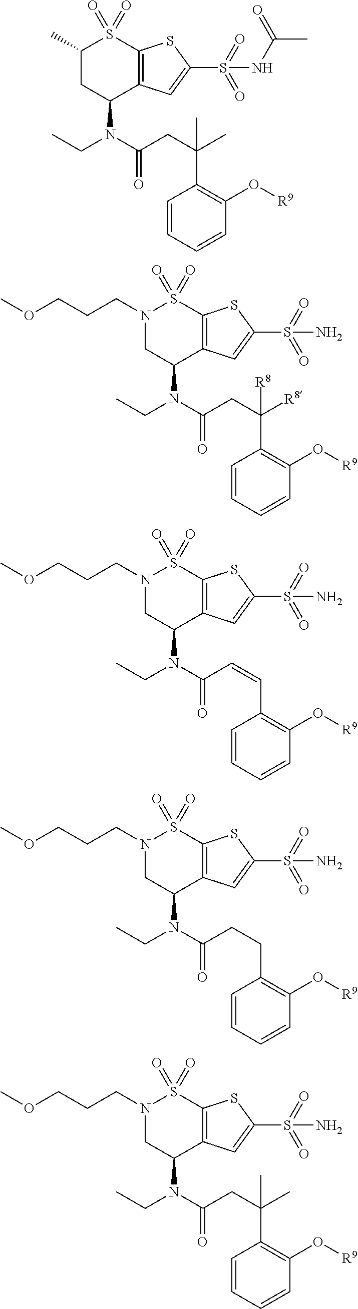

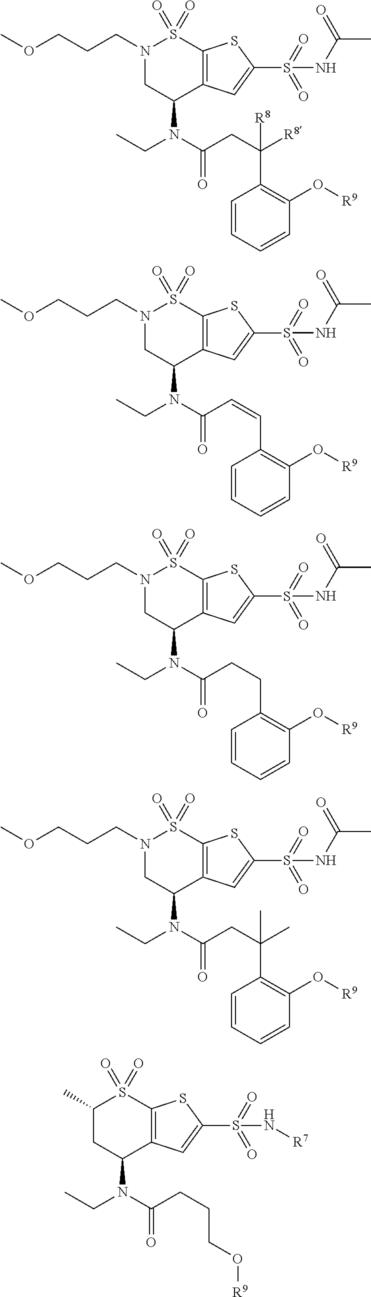

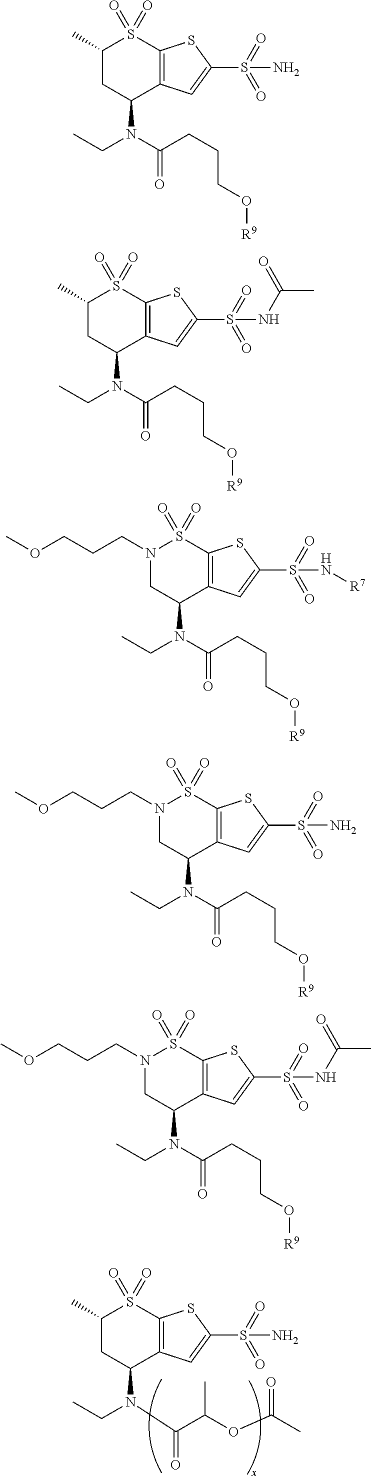

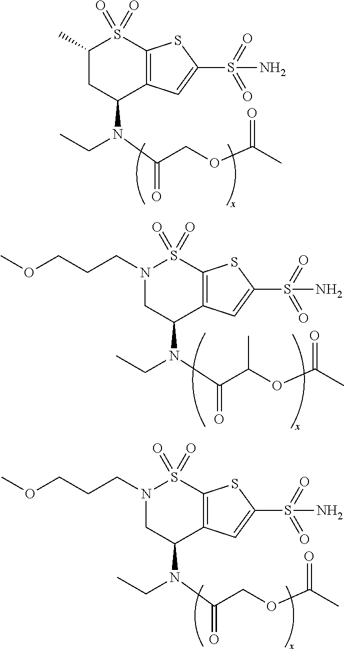

In one embodiment R⁹ is

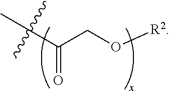

In one embodiment R⁹ is

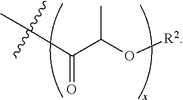

In one embodiment R⁹ is

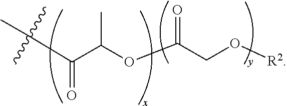

In one embodiment R⁹ is

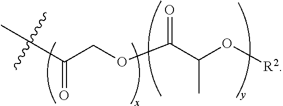

In one embodiment x is 1 and y is 1.

In one embodiment x is 1 and y is 2.

In one embodiment x is 1 and y is 3.

In one embodiment x is 1 and y is 4.

In one embodiment x is 1 and y is 5.

In one embodiment x is 1 and y is 6.

In one embodiment x is 1 and y is 7.

In one embodiment x is 1 and y is 8.

In one embodiment x is 2 and y is 1.

In one embodiment x is 2 and y is 2.

In one embodiment x is 2 and y is 3.

In one embodiment x is 2 and y is 4.

In one embodiment x is 2 and y is 5.

In one embodiment x is 2 and y is 6.

In one embodiment x is 2 and y is 7.

In one embodiment x is 2 and y is 8.

In one embodiment x is 3 and y is 1.

In one embodiment x is 3 and y is 2.

In one embodiment x is 3 and y is 3.

In one embodiment x is 3 and y is 4.

In one embodiment x is 3 and y is 5.

In one embodiment x is 3 and y is 6.

In one embodiment x is 3 and y is 7.

In one embodiment x is 3 and y is 8.

In one embodiment x is 4 and y is 1.

In one embodiment x is 4 and y is 2.

In one embodiment x is 4 and y is 3.

In one embodiment x is 4 and y is 4.

In one embodiment x is 4 and y is 5.

In one embodiment x is 4 and y is 6.

In one embodiment x is 4 and y is 7.

In one embodiment x is 4 and y is 8.

In one embodiment x is 5 and y is 1.

In one embodiment x is 5 and y is 2.

In one embodiment x is 5 and y is 3.

In one embodiment x is 5 and y is 4.

In one embodiment x is 5 and y is 5.

In one embodiment x is 5 and y is 6.

In one embodiment x is 5 and y is 7.

In one embodiment x is 5 and y is 8.

In one embodiment x is 6 and y is 1.

In one embodiment x is 6 and y is 2.

In one embodiment x is 6 and y is 3.

In one embodiment x is 6 and y is 4.

In one embodiment x is 6 and y is 5.

In one embodiment x is 6 and y is 6.

In one embodiment x is 6 and y is 7.

In one embodiment x is 6 and y is 8.

In one embodiment x is 7 and y is 1.

In one embodiment x is 7 and y is 2.

In one embodiment x is 7 and y is 3.

In one embodiment x is 7 and y is 4.

In one embodiment x is 7 and y is 5.

In one embodiment x is 7 and y is 6.

In one embodiment x is 7 and y is 7.

In one embodiment x is 7 and y is 8.

In one embodiment x is 8 and y is 1.

In one embodiment x is 8 and y is 2.

In one embodiment x is 8 and y is 3.

In one embodiment x is 8 and y is 4.

In one embodiment x is 8 and y is 5.

In one embodiment x is 8 and y is 6.

In one embodiment x is 8 and y is 7.

In one embodiment x is 8 and y is 8.

### Example 4. Non-Limiting Examples of Compounds of Formula VI and Formula VII

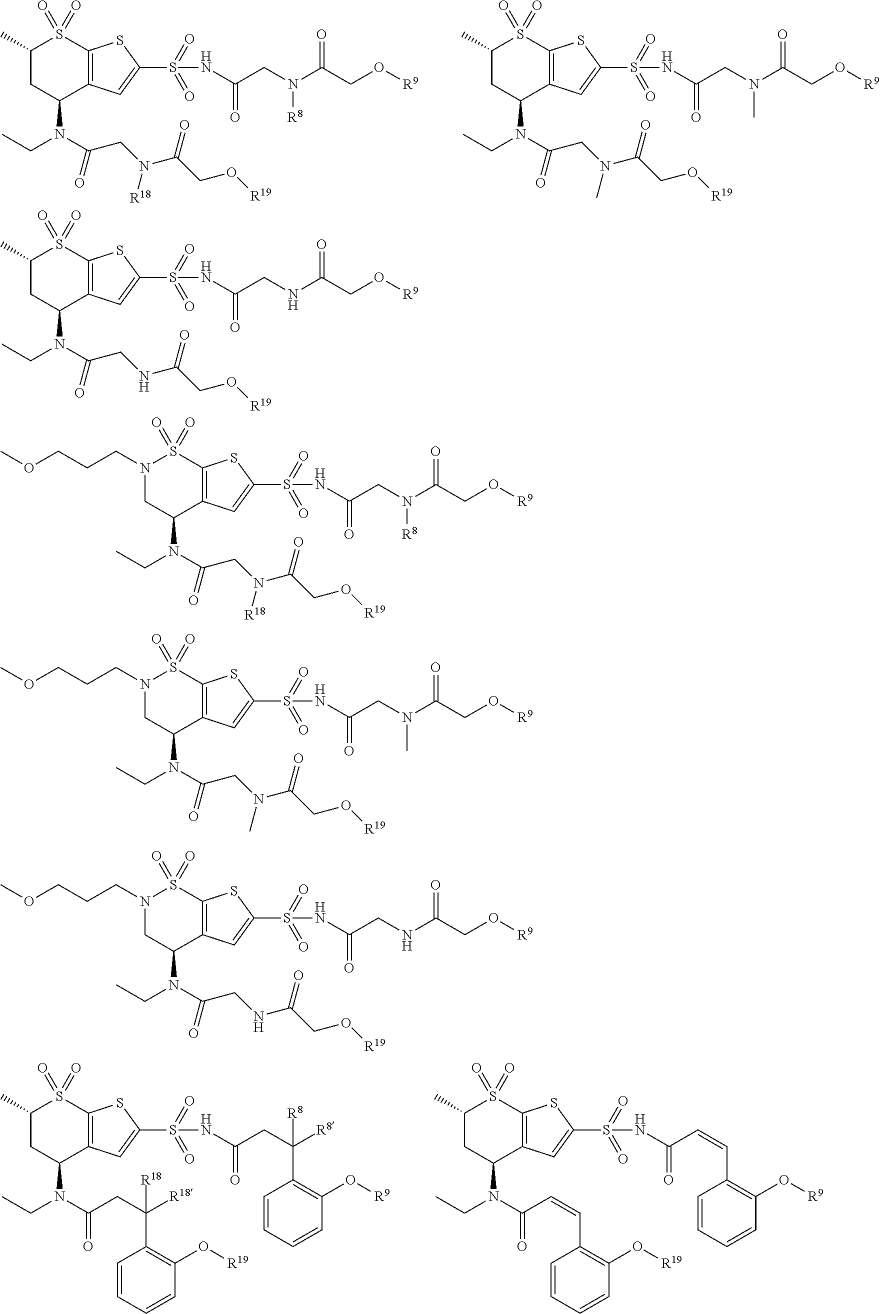

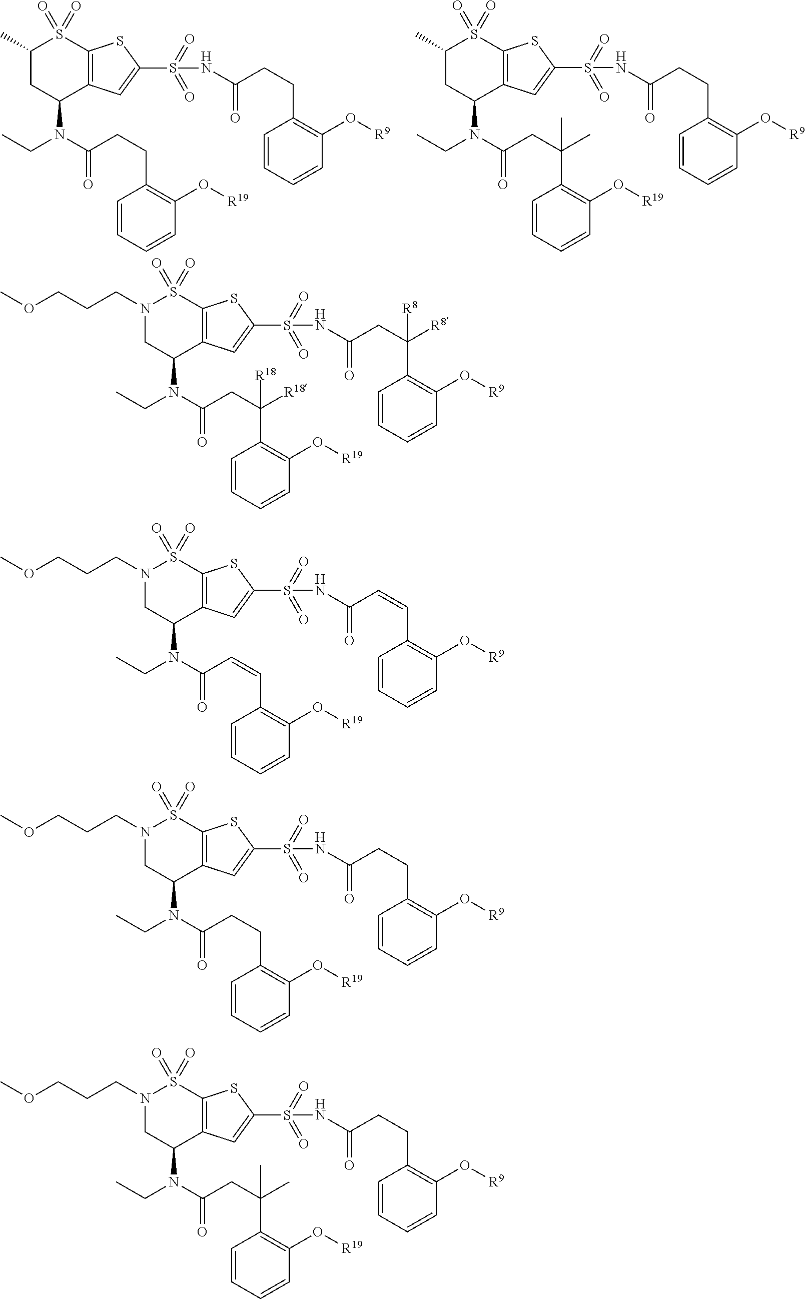

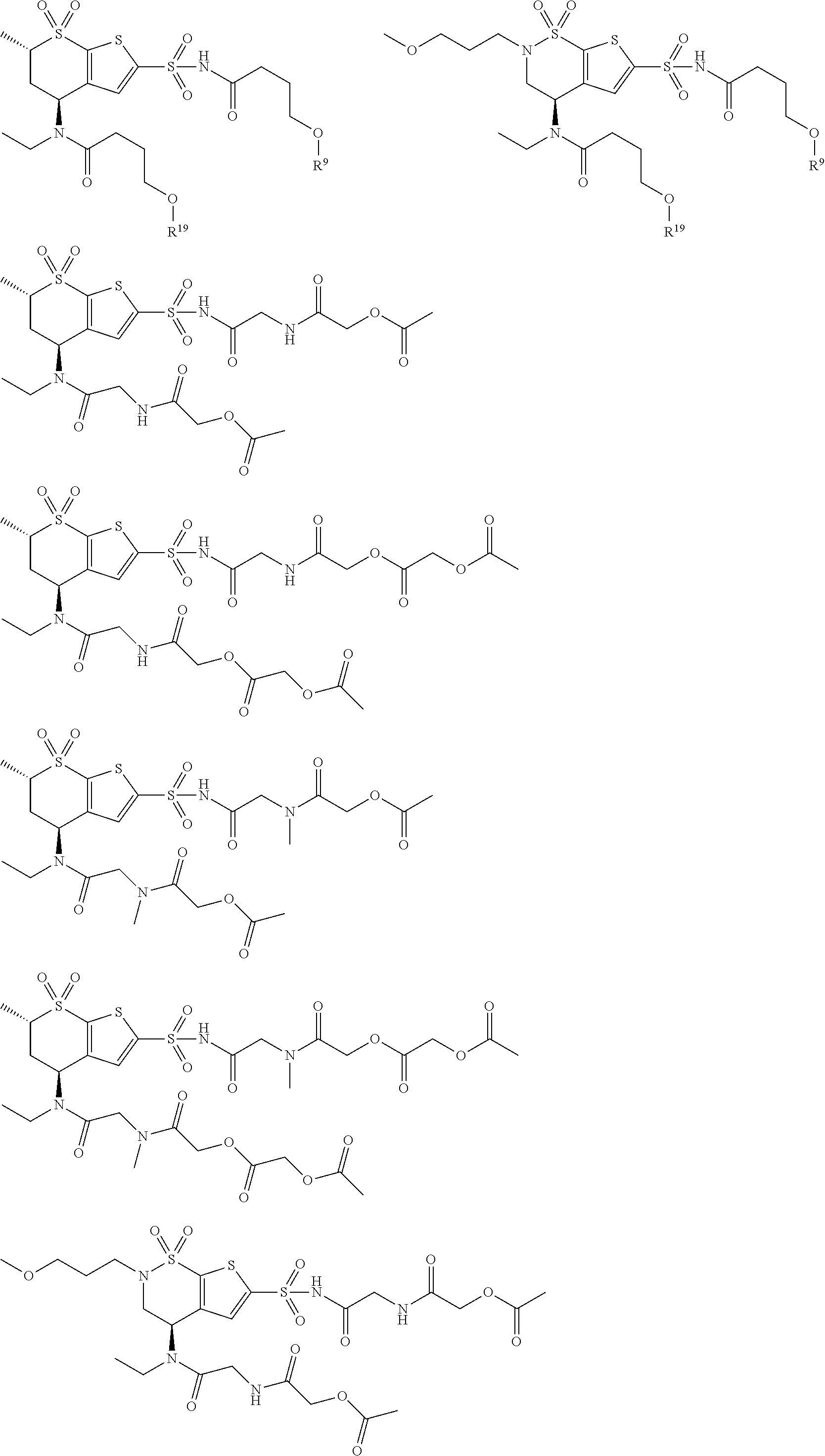

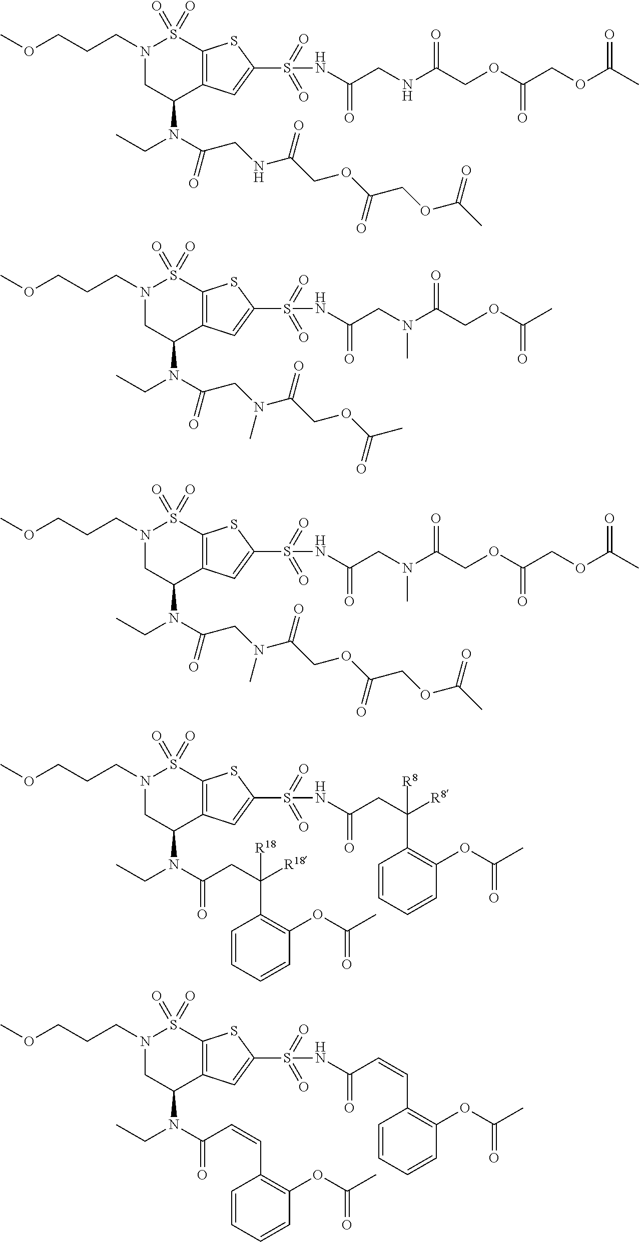

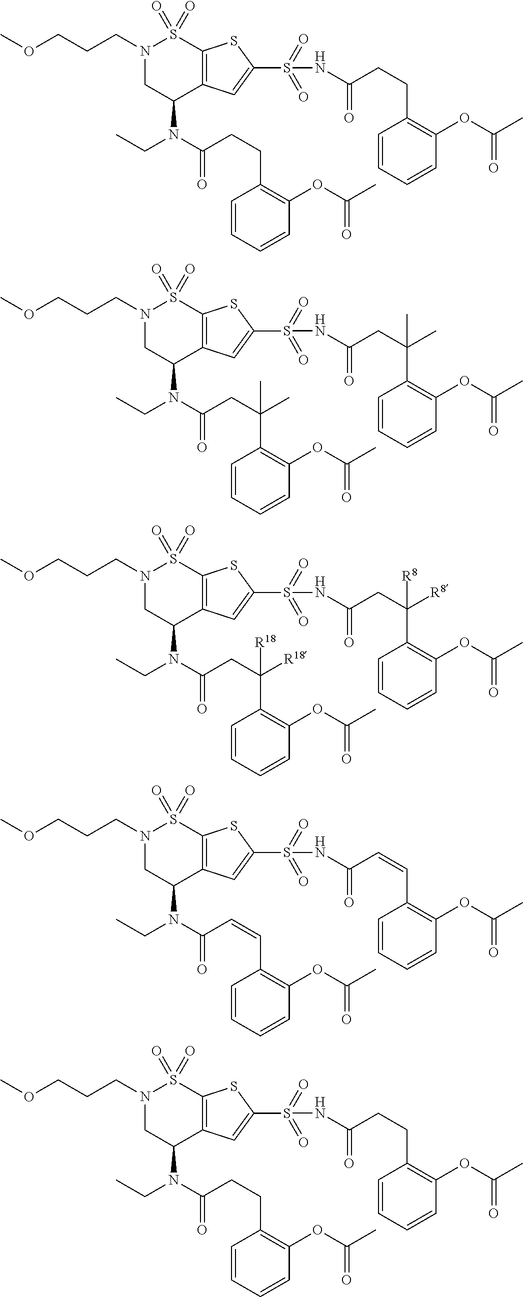

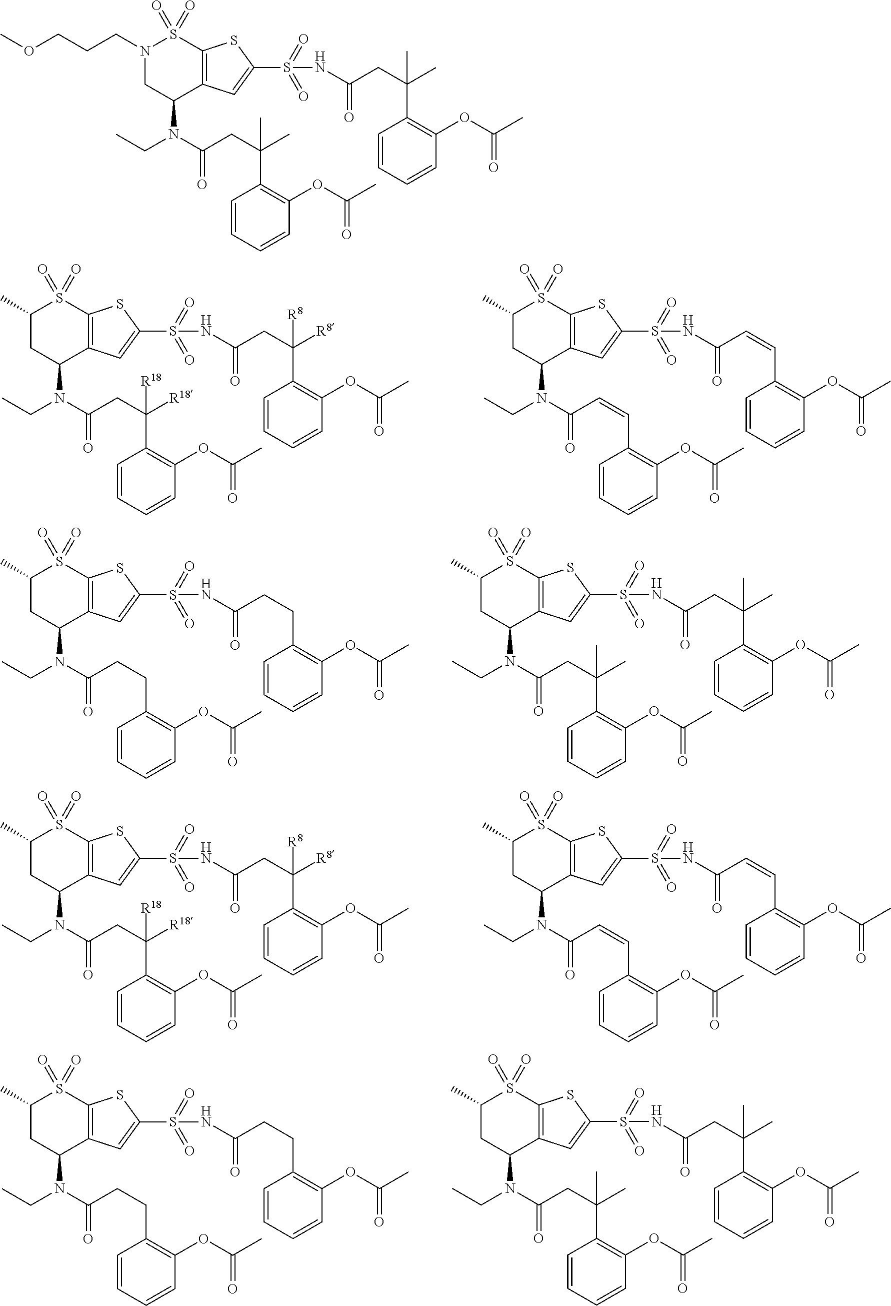

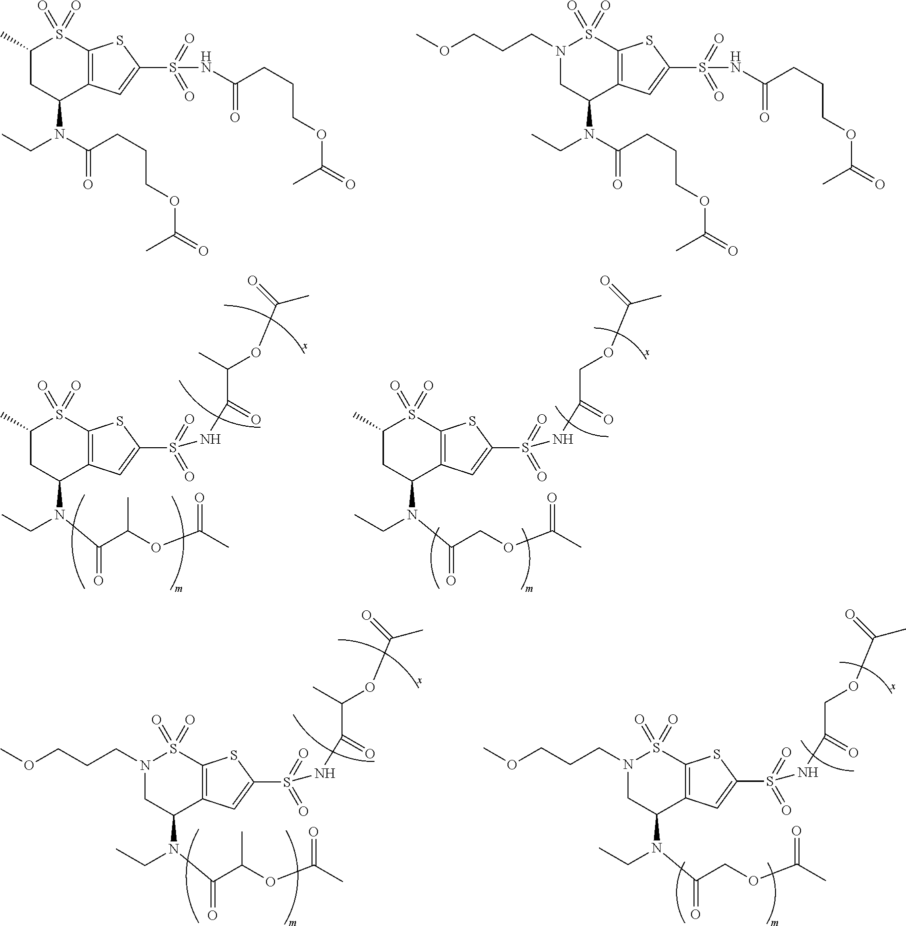

In one embodiment R⁹ is

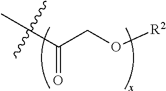

### and R¹⁹ is

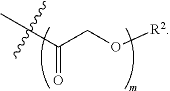

In one embodiment R⁹ is

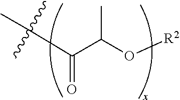

### and R¹⁹ is

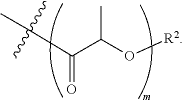

In one embodiment x is 1 and m is 1.

In one embodiment x is 1 and m is 2.

In one embodiment x is 1 and m is 3.

In one embodiment x is 1 and m is 4.

In one embodiment x is 1 and m is 5.

In one embodiment x is 1 and m is 6.

In one embodiment x is 1 and m is 7.

In one embodiment x is 1 and m is 8.

In one embodiment x is 2 and m is 1.

In one embodiment x is 2 and m is 2.

In one embodiment x is 2 and m is 3.

In one embodiment x is 2 and m is 4.

In one embodiment x is 2 and m is 5.

In one embodiment x is 2 and m is 6.

In one embodiment x is 2 and m is 7.

In one embodiment x is 2 and m is 8.

In one embodiment x is 3 and m is 1.

In one embodiment x is 3 and m is 2.

In one embodiment x is 3 and m is 3.

In one embodiment x is 3 and m is 4.

In one embodiment x is 3 and m is 5.

In one embodiment x is 3 and m is 6.

In one embodiment x is 3 and m is 7.

In one embodiment x is 3 and m is 8.

In one embodiment x is 4 and m is 1.

In one embodiment x is 4 and m is 2.

In one embodiment x is 4 and m is 3.

In one embodiment x is 4 and m is 4.

In one embodiment x is 4 and m is 5.

In one embodiment x is 4 and m is 6.

In one embodiment x is 4 and m is 7.

In one embodiment x is 4 and m is 8.

In one embodiment x is 5 and m is 1.

In one embodiment x is 5 and m is 2.

In one embodiment x is 5 and m is 3.

In one embodiment x is 5 and m is 4.

In one embodiment x is 5 and m is 5.

In one embodiment x is 5 and m is 6.

In one embodiment x is 5 and m is 7.

In one embodiment x is 5 and m is 8.

In one embodiment x is 6 and m is 1.

In one embodiment x is 6 and m is 2.

In one embodiment x is 6 and m is 3.

In one embodiment x is 6 and m is 4.

In one embodiment x is 6 and m is 5.

In one embodiment x is 6 and m is 6.

In one embodiment x is 6 and m is 7.

In one embodiment x is 6 and m is 8.

In one embodiment x is 7 and m is 1.

In one embodiment x is 7 and m is 2.

In one embodiment x is 7 and m is 3.

In one embodiment x is 7 and m is 4.

In one embodiment x is 7 and m is 5.

In one embodiment x is 7 and m is 6.

In one embodiment x is 7 and m is 7.

In one embodiment x is 7 and m is 8.

In one embodiment x is 8 and m is 1.

In one embodiment x is 8 and m is 2.

In one embodiment x is 8 and m is 3.

In one embodiment x is 8 and m is 4.

In one embodiment x is 8 and m is 5.

In one embodiment x is 8 and m is 6.

In one embodiment x is 8 and m is 7.

In one embodiment x is 8 and m is 8.

### Example 5. Non-Limiting Examples of Compounds of Formula IX, Formula X, Formula XI and Formula XII

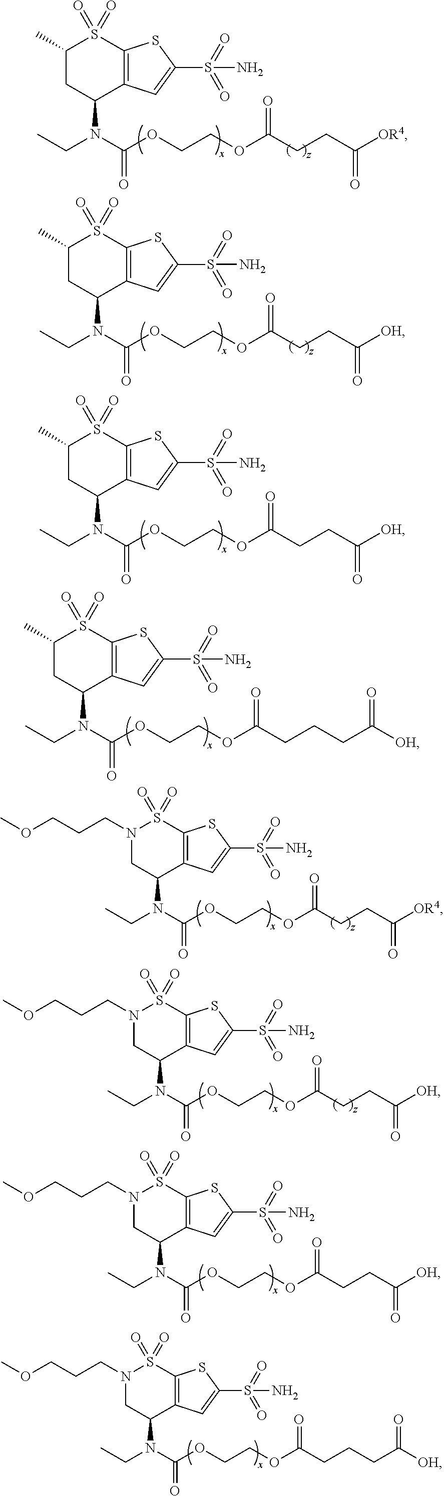

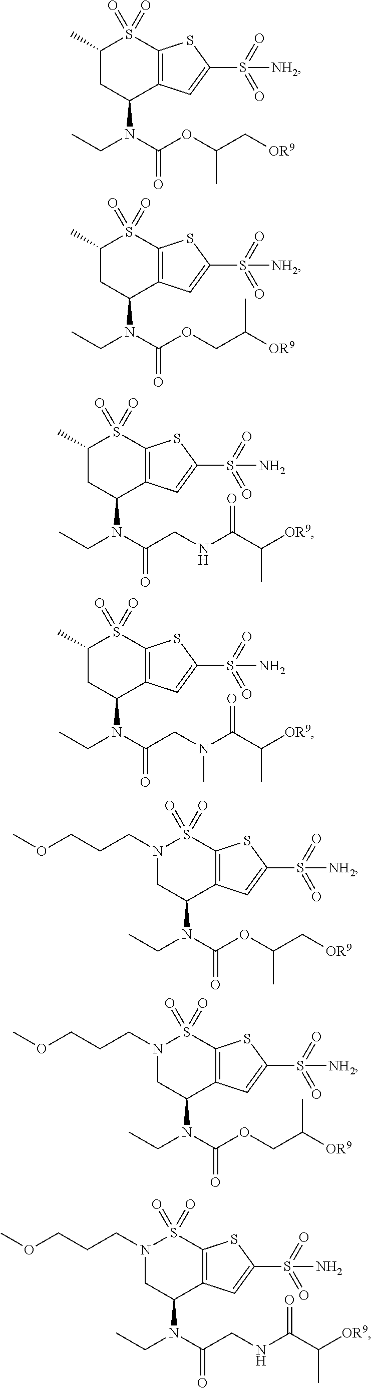

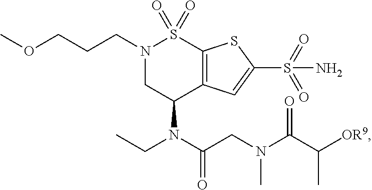

In one embodiment R⁹ is

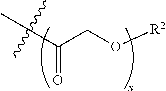

and x is 1.

In one embodiment R⁹ is

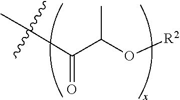

and x is 1.

In one embodiment, x is 1, 2, 3, 4, 5, 6, 7, or 8. In one embodiment, x is 1, 2, 3, or 4. In one embodiment, z is 1, 2, 3, 4, 5, 6, 7, or 8. In one embodiment, z is 1, 2, 3, or 4.

### Example 6. Non-Limiting Examples of Compounds of Formula XIII, Formula XIV, Formula XV and Formula XVI

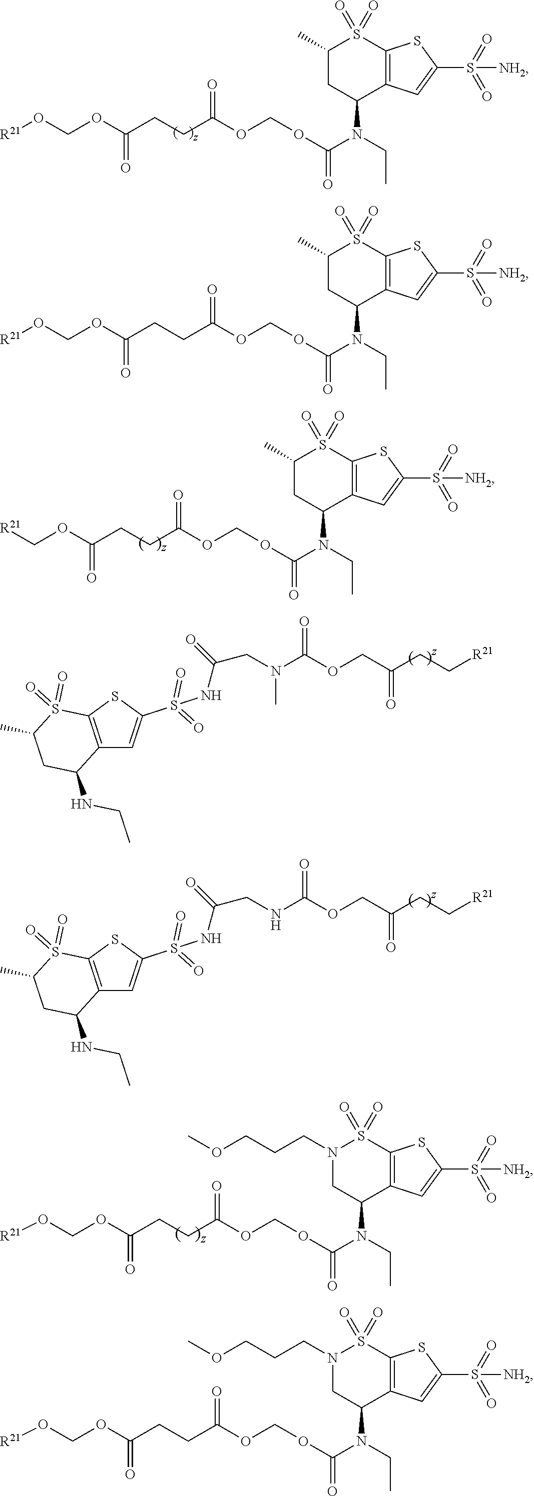

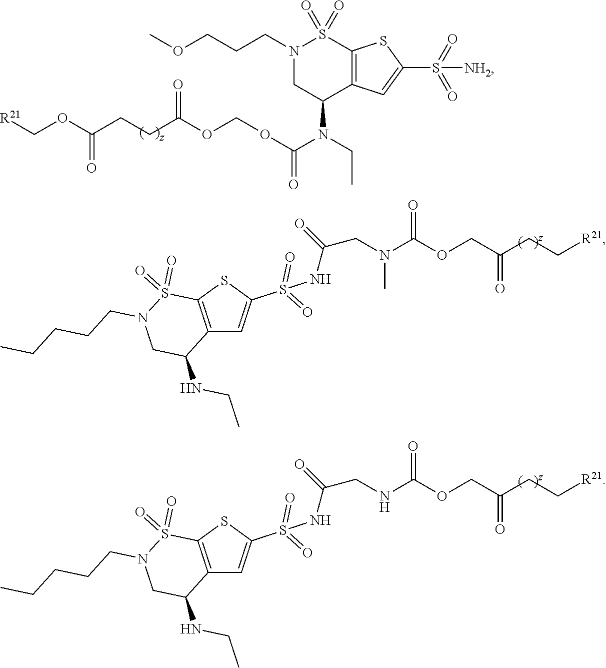

In one embodiment, R²¹ is

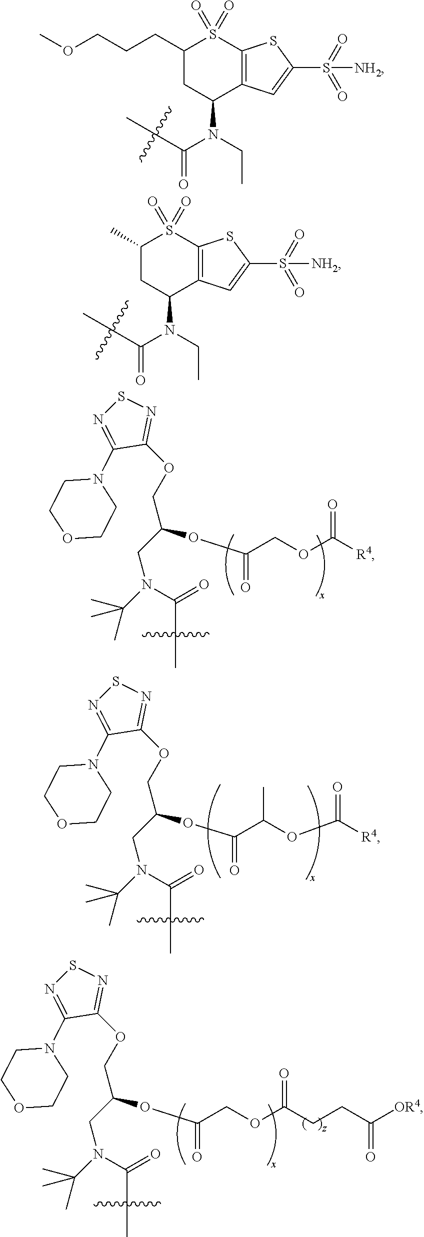

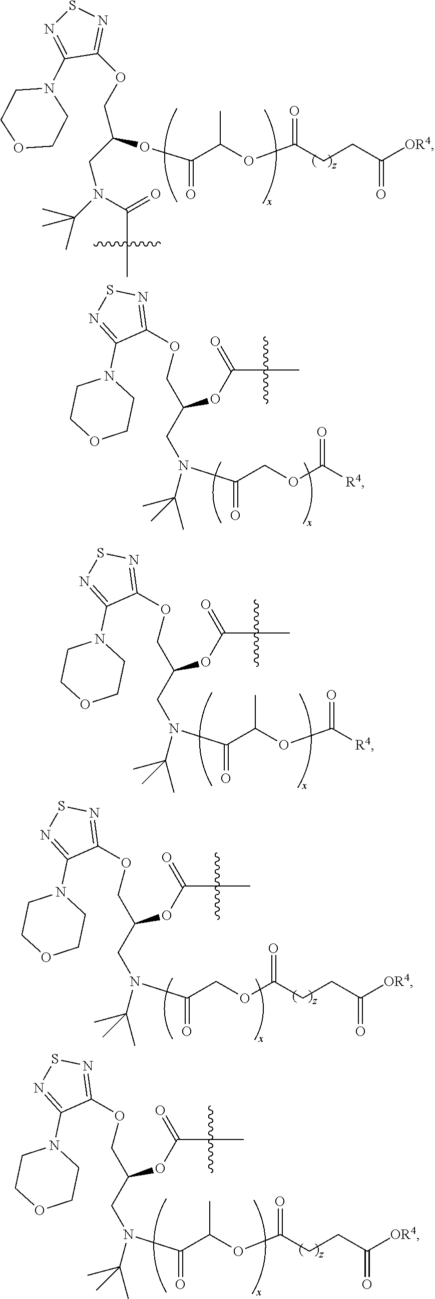

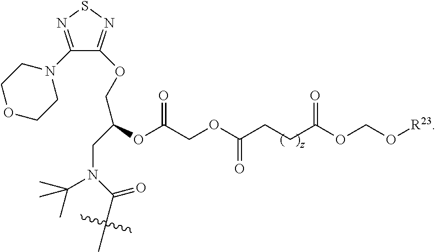

In one embodiment, z is selected from 1, 2, 3, 4, 5, and 6. In one embodiment, z is selected from 1, 2, and 3. In one embodiment, z is selected from 1 and 2.

In one embodiment, R⁴ is alkyl or aryl. In one embodiment, R⁴ is methyl. In one embodiment, R⁴ is hydrogen.

### Example 7. Non-Limiting Examples of Compounds of the Present Invention

Table 1 shows illustrative compounds of Formula II, Formula III, Formula IV, and Formula V. Table 2 shows illustrative compounds of Formula IV, Formula IX, Formula X, Formula XI, Formula XII, Formula XIII, Formula XIV, Formula XV, and Formula XVI

TABLE 1
Non-limiting Examples of Compounds of Formula II, Formula III, Formula IV,
and Formula V

| Compd No. | Structure |
|---|---|
| 1-1 | 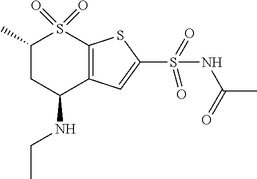 [SMILES: CCN[C@H]1C[C@H](C)S(=O)(=O)c2sc(S(=O)(=O)NC(C)=O)cc21] |
| 2-1 | 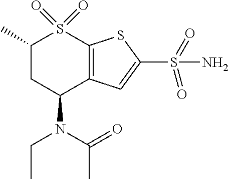 [SMILES: CCN(C(C)=O)[C@H]1C[C@H](C)S(=O)(=O)c2sc(S(N)(=O)=O)cc21] |
| 3-1 | 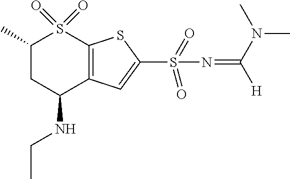 |
| 4-2 | 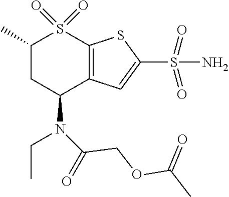 [SMILES: CCN(C(=O)COC(C)=O)[C@H]1C[C@H](C)S(=O)(=O)c2sc(S(N)(=O)=O)cc21] |
| 5-6 | 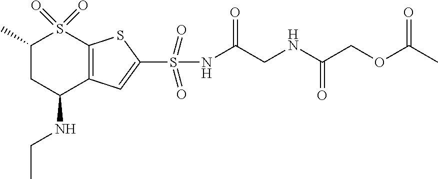 [SMILES: CCN[C@H]1C[C@H](C)S(=O)(=O)c2sc(S(=O)(=O)NC(=O)CNC(=O)COC(C)=O)cc21] |
| 6-5 | 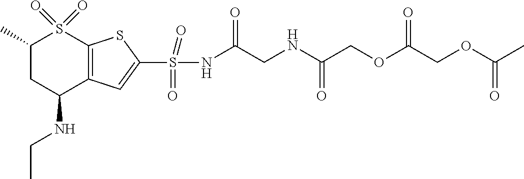 [SMILES: CCN[C@H]1C[C@H](C)S(=O)(=O)c2sc(S(=O)(=O)NC(=O)CNC(=O)COC(=O)COC(C)=O)cc21] |
| 7-6 | 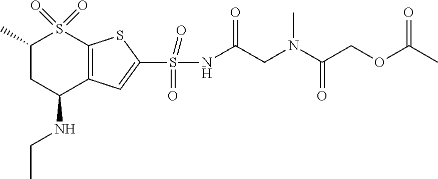 [SMILES: CCN[C@H]1C[C@H](C)S(=O)(=O)c2sc(S(=O)(=O)NC(=O)CN(C)C(=O)COC(C)=O)cc21] |
| 9-7 | 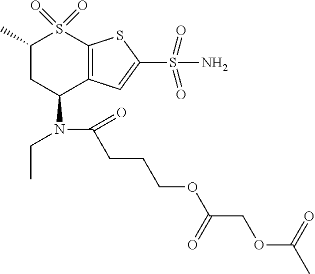 [SMILES: CCN(C(=O)CCCOC(=O)COC(C)=O)[C@H]1C[C@H](C)S(=O)(=O)c2sc(S(N)(=O)=O)cc21] |
| 10-4 | 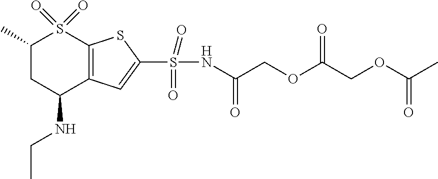 [SMILES: CCN[C@H]1C[C@H](C)S(=O)(=O)c2sc(S(=O)(=O)NC(=O)COC(=O)COC(C)=O)cc21] |
| 11-1 | 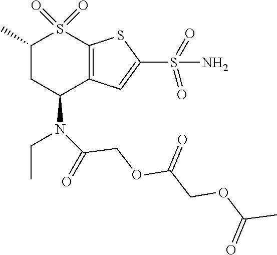 [SMILES: CCN(C(=O)COC(=O)COC(C)=O)[C@H]1C[C@H](C)S(=O)(=O)c2sc(S(N)(=O)=O)cc21] |
| 12-3 | 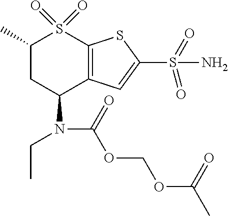 [SMILES: CCN(C(=O)OCOC(C)=O)[C@H]1C[C@H](C)S(=O)(=O)c2sc(S(N)(=O)=O)cc21] |
| 13-1 | 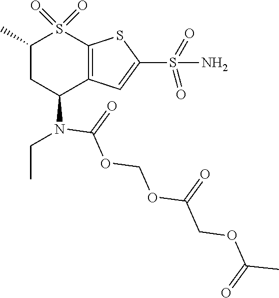 [SMILES: CCN(C(=O)OCOC(=O)COC(C)=O)[C@H]1C[C@H](C)S(=O)(=O)c2sc(S(N)(=O)=O)cc21] |
| 14-5 | 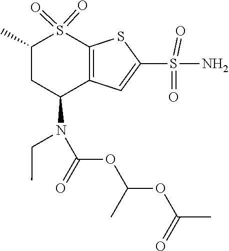 [SMILES: CCN(C(=O)OC(C)OC(C)=O)[C@H]1C[C@H](C)S(=O)(=O)c2sc(S(N)(=O)=O)cc21] |
| 15-2 | 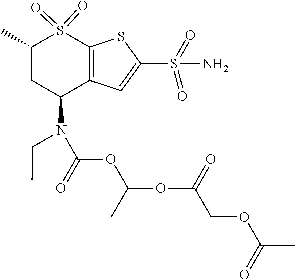 [SMILES: CCN(C(=O)OC(C)OC(=O)COC(C)=O)[C@H]1C[C@H](C)S(=O)(=O)c2sc(S(N)(=O)=O)cc21] |
| 16-2 | 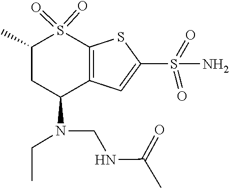 [SMILES: CCN(CNC(C)=O)[C@H]1C[C@H](C)S(=O)(=O)c2sc(S(N)(=O)=O)cc21] |
| 17-10 | 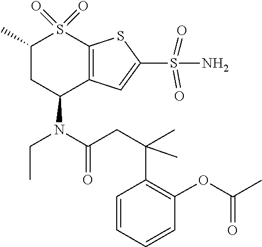 [SMILES: CCN(C(=O)CC(C)(C)c1ccccc1OC(C)=O)[C@H]1C[C@H](C)S(=O)(=O)c2sc(S(N)(=O)=O)cc21] |
| 18-3 | 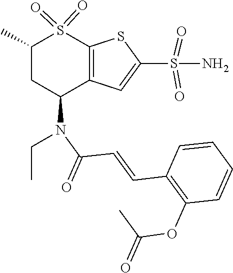 [SMILES: CCN(C(=O)/C=C/c1ccccc1OC(C)=O)[C@H]1C[C@H](C)S(=O)(=O)c2sc(S(N)(=O)=O)cc21] |
| 19-2 | 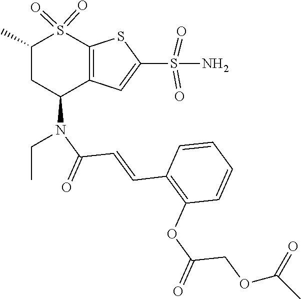 [SMILES: CCN(C(=O)/C=C/c1ccccc1OC(=O)COC(C)=O)[C@H]1C[C@H](C)S(=O)(=O)c2sc(S(N)(=O)=O)cc21] |
| 20-1 | 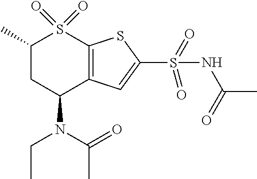 [SMILES: CCN(C(C)=O)[C@H]1C[C@H](C)S(=O)(=O)c2sc(S(=O)(=O)NC(C)=O)cc21] |
| 21-1 | 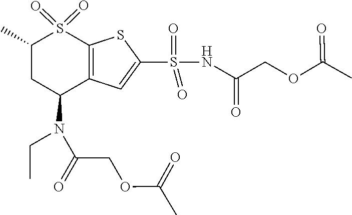 [SMILES: CCN(C(=O)COC(C)=O)[C@H]1C[C@H](C)S(=O)(=O)c2sc(S(=O)(=O)NC(=O)COC(C)=O)cc21] |
| 22-4 | 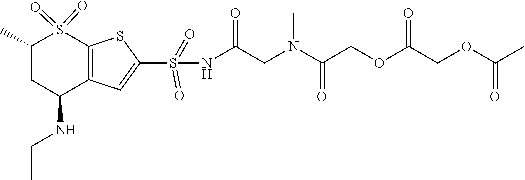 [SMILES: CCN[C@H]1C[C@H](C)S(=O)(=O)c2sc(S(=O)(=O)NC(=O)CN(C)C(=O)COC(=O)COC(C)=O)cc21] |
| 23-1 | 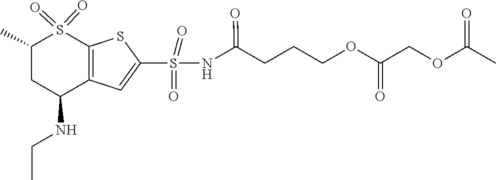 [SMILES: CCN[C@H]1C[C@H](C)S(=O)(=O)c2sc(S(=O)(=O)NC(=O)CCCOC(=O)COC(C)=O)cc21] |
| 24-1 | 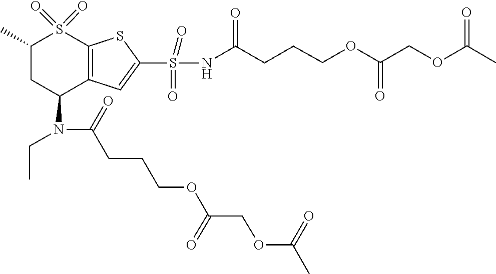 [SMILES: CCN(C(=O)CCCOC(=O)COC(C)=O)[C@H]1C[C@H](C)S(=O)(=O)c2sc(S(=O)(=O)NC(=O)CCCOC(=O)COC(C)=O)cc21] |
| 25-1 | 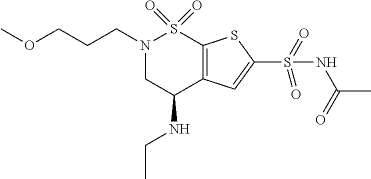 [SMILES: CCN[C@H]1CN(CCCOC)S(=O)(=O)c2sc(S(=O)(=O)NC(C)=O)cc21] |
| 26-1 | 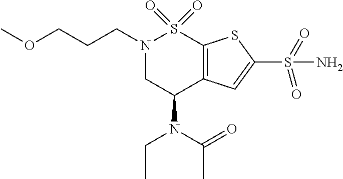 [SMILES: CCN(C(C)=O)[C@H]1CN(CCCOC)S(=O)(=O)c2sc(S(N)(=O)=O)cc21] |
| 27-1 | 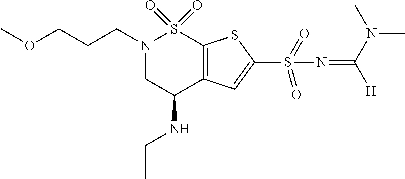 |
| 28-1 | 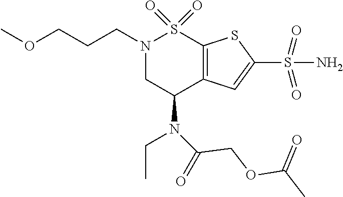 [SMILES: CCN(C(=O)COC(C)=O)[C@H]1CN(CCCOC)S(=O)(=O)c2sc(S(N)(=O)=O)cc21] |
| 29-1 | 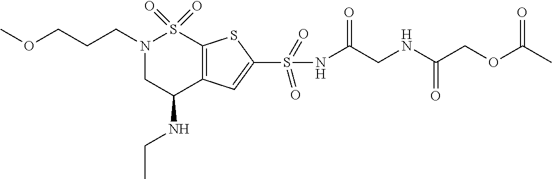 [SMILES: CCN[C@H]1CN(CCCOC)S(=O)(=O)c2sc(S(=O)(=O)NC(=O)CNC(=O)COC(C)=O)cc21] |
| 30-1 | 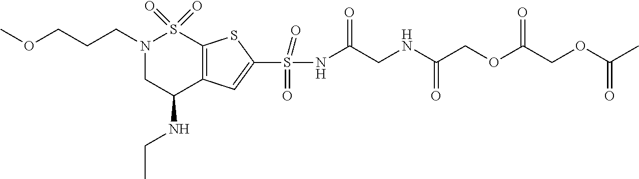 [SMILES: CCN[C@H]1CN(CCCOC)S(=O)(=O)c2sc(S(=O)(=O)NC(=O)CNC(=O)COC(=O)COC(C)=O)cc21] |
| 31-1 | 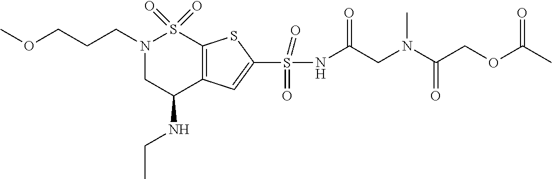 [SMILES: CCN[C@H]1CN(CCCOC)S(=O)(=O)c2sc(S(=O)(=O)NC(=O)CN(C)C(=O)COC(C)=O)cc21] |
| 32-1 | 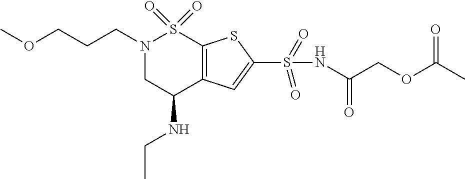 [SMILES: CCN[C@H]1CN(CCCOC)S(=O)(=O)c2sc(S(=O)(=O)NC(=O)COC(C)=O)cc21] |
| 33-1 | 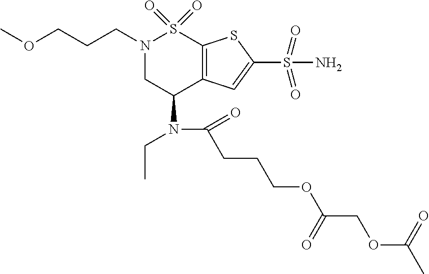 [SMILES: CCN(C(=O)CCCOC(=O)COC(C)=O)[C@H]1CN(CCCOC)S(=O)(=O)c2sc(S(N)(=O)=O)cc21] |
| 34-1 | 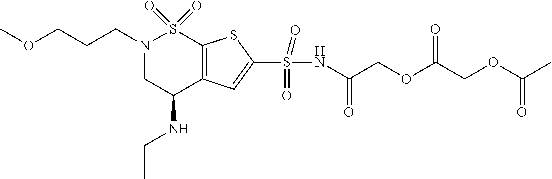 [SMILES: CCN[C@H]1CN(CCCOC)S(=O)(=O)c2sc(S(=O)(=O)NC(=O)COC(=O)COC(C)=O)cc21] |
| 35-1 | 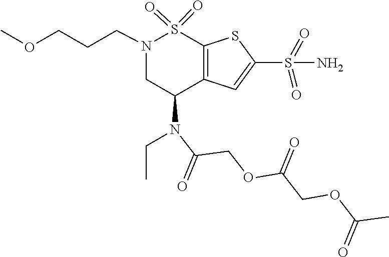 [SMILES: CCN(C(=O)COC(=O)COC(C)=O)[C@H]1CN(CCCOC)S(=O)(=O)c2sc(S(N)(=O)=O)cc21] |
| 36-1 | 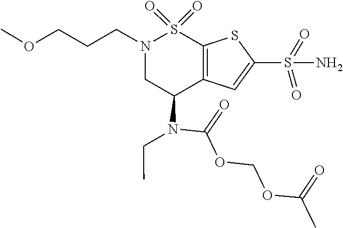 [SMILES: CCN(C(=O)OCOC(C)=O)[C@H]1CN(CCCOC)S(=O)(=O)c2sc(S(N)(=O)=O)cc21] |
| 37-1 | 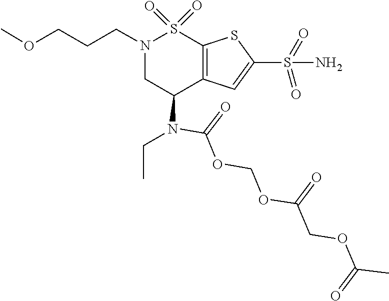 [SMILES: CCN(C(=O)OCOC(=O)COC(C)=O)[C@H]1CN(CCCOC)S(=O)(=O)c2sc(S(N)(=O)=O)cc21] |
| 38-1 | 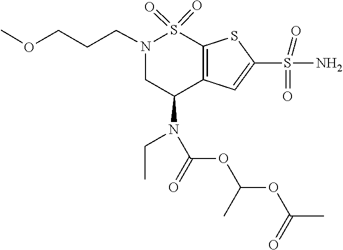 [SMILES: CCN(C(=O)OC(C)OC(C)=O)[C@H]1CN(CCCOC)S(=O)(=O)c2sc(S(N)(=O)=O)cc21] |
| 39-1 | 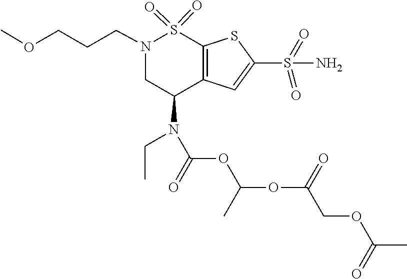 [SMILES: CCN(C(=O)OC(C)OC(=O)COC(C)=O)[C@H]1CN(CCCOC)S(=O)(=O)c2sc(S(N)(=O)=O)cc21] |
| 40-1 | 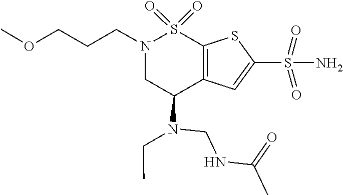 [SMILES: CCN(CNC(C)=O)[C@H]1CN(CCCOC)S(=O)(=O)c2sc(S(N)(=O)=O)cc21] |
| 41-1 | 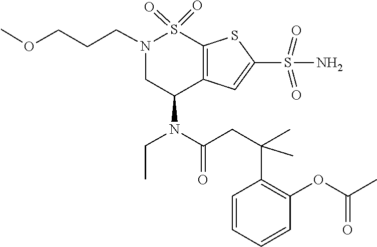 [SMILES: CCN(C(=O)CC(C)(C)c1ccccc1OC(C)=O)[C@H]1CN(CCCOC)S(=O)(=O)c2sc(S(N)(=O)=O)cc21] |
| 42-1 | 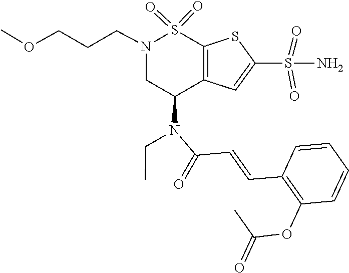 [SMILES: CCN(C(=O)/C=C/c1ccccc1OC(C)=O)[C@H]1CN(CCCOC)S(=O)(=O)c2sc(S(N)(=O)=O)cc21] |
| 43-1 | 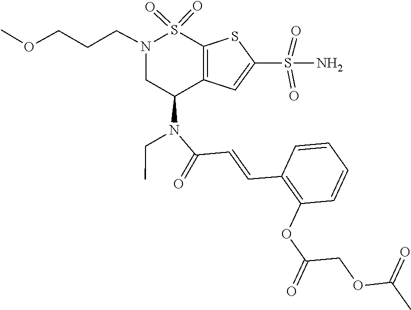 [SMILES: CCN(C(=O)/C=C/c1ccccc1OC(=O)COC(C)=O)[C@H]1CN(CCCOC)S(=O)(=O)c2sc(S(N)(=O)=O)cc21] |
| 44-1 | 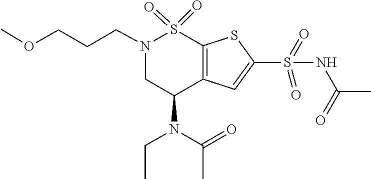 [SMILES: CCN(C(C)=O)[C@H]1CN(CCCOC)S(=O)(=O)c2sc(S(=O)(=O)NC(C)=O)cc21] |
| 45-1 | 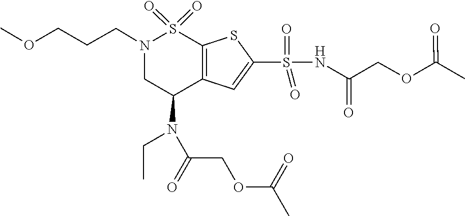 [SMILES: CCN(C(=O)COC(C)=O)[C@H]1CN(CCCOC)S(=O)(=O)c2sc(S(=O)(=O)NC(=O)COC(C)=O)cc21] |
| 46-1 | 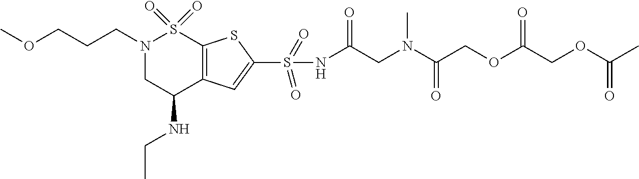 [SMILES: CCN[C@H]1CN(CCCOC)S(=O)(=O)c2sc(S(=O)(=O)NC(=O)CN(C)C(=O)COC(=O)COC(C)=O)cc21] |
| 47-1 | 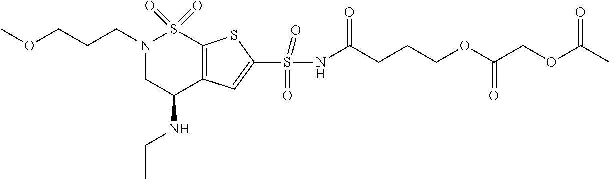 [SMILES: CCN[C@H]1CN(CCCOC)S(=O)(=O)c2sc(S(=O)(=O)NC(=O)CCCOC(=O)COC(C)=O)cc21] |
| 48-1 | 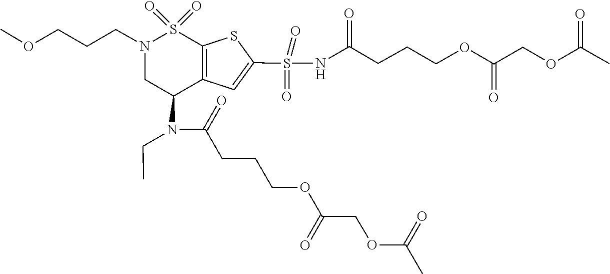 [SMILES: CCN(C(=O)CCCOC(=O)COC(C)=O)[C@H]1CN(CCCOC)S(=O)(=O)c2sc(S(=O)(=O)NC(=O)CCCOC(=O)COC(C)=O)cc21] |

TABLE 2
Non-limiting Examples of Compounds of Formula IV, Formula IX, Formula XI,
Formula XIII, and Formula XV

| Compd No. | Structure |
|---|---|
| 49-1 | 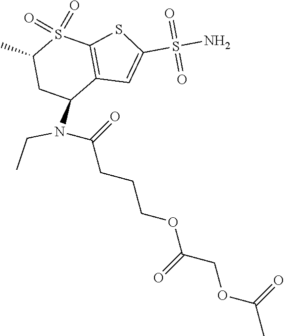 [SMILES: CCN(C(=O)CCCOC(=O)COC(C)=O)[C@H]1C[C@H](C)S(=O)(=O)c2sc(S(N)(=O)=O)cc21] |
| 50-1 | 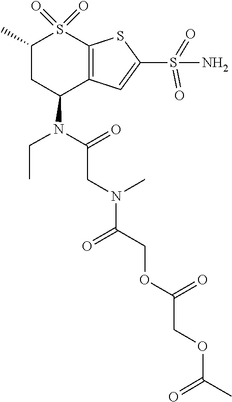 [SMILES: CCN(C(=O)CN(C)C(=O)COC(=O)COC(C)=O)[C@H]1C[C@H](C)S(=O)(=O)c2sc(S(N)(=O)=O)cc21] |
| 51-1 | 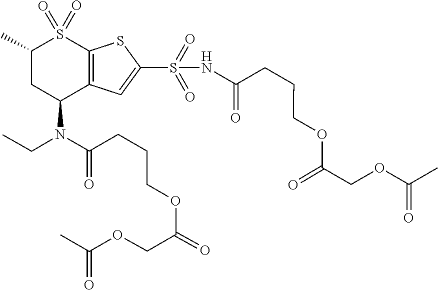 [SMILES: CCN(C(=O)CCCOC(=O)COC(C)=O)[C@H]1C[C@H](C)S(=O)(=O)c2sc(S(=O)(=O)NC(=O)CCCOC(=O)COC(C)=O)cc21] |
| 52-5 | 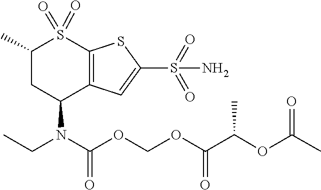 [SMILES: CCN(C(=O)OCOC(=O)[C@H](C)OC(C)=O)[C@H]1C[C@H](C)S(=O)(=O)c2sc(S(N)(=O)=O)cc21] |
| 53-5 | 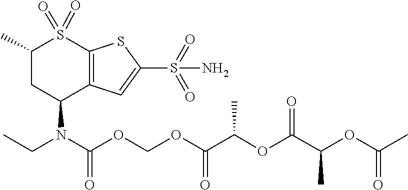 [SMILES: CCN(C(=O)OCOC(=O)[C@H](C)OC(=O)[C@H](C)OC(C)=O)[C@H]1C[C@H](C)S(=O)(=O)c2sc(S(N)(=O)=O)cc21] |
| 54-5 | 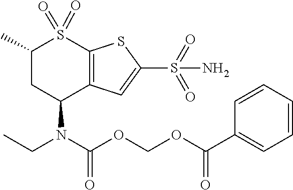 [SMILES: CCN(C(=O)OCOC(=O)c1ccccc1)[C@H]1C[C@H](C)S(=O)(=O)c2sc(S(N)(=O)=O)cc21] |
| 55-5 | 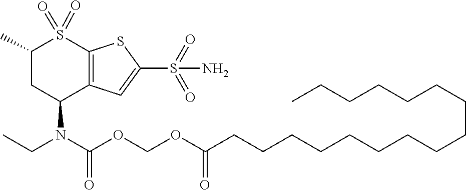 [SMILES: CCCCCCCCCCCCCCCCC(=O)OCOC(=O)N(CC)[C@H]1C[C@H](C)S(=O)(=O)c2sc(S(N)(=O)=O)cc21] |
| 56-7 | 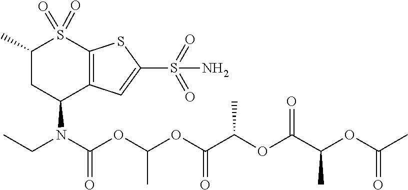 [SMILES: CCN(C(=O)OC(C)OC(=O)[C@H](C)OC(=O)[C@H](C)OC(C)=O)[C@H]1C[C@H](C)S(=O)(=O)c2sc(S(N)(=O)=O)cc21] |
| 57-7 | 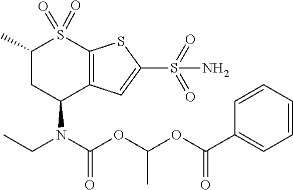 [SMILES: CCN(C(=O)OC(C)OC(=O)c1ccccc1)[C@H]1C[C@H](C)S(=O)(=O)c2sc(S(N)(=O)=O)cc21] |
| 58-7 | 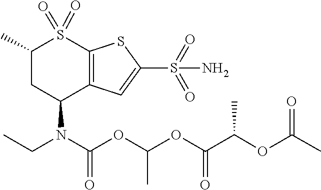 [SMILES: CCN(C(=O)OC(C)OC(=O)[C@H](C)OC(C)=O)[C@H]1C[C@H](C)S(=O)(=O)c2sc(S(N)(=O)=O)cc21] |
| 59-6 | 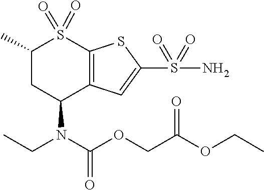 [SMILES: CCOC(=O)COC(=O)N(CC)[C@H]1C[C@H](C)S(=O)(=O)c2sc(S(N)(=O)=O)cc21] |
| 60-5 | 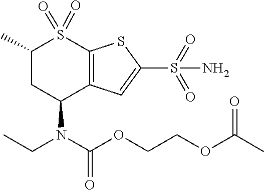 [SMILES: CCN(C(=O)OCCOC(C)=O)[C@H]1C[C@H](C)S(=O)(=O)c2sc(S(N)(=O)=O)cc21] |
| 61-8 | 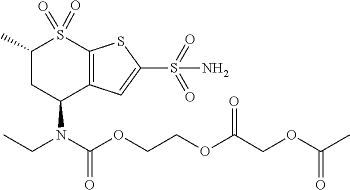 [SMILES: CCN(C(=O)OCCOC(=O)COC(C)=O)[C@H]1C[C@H](C)S(=O)(=O)c2sc(S(N)(=O)=O)cc21] |
| 62-7 | 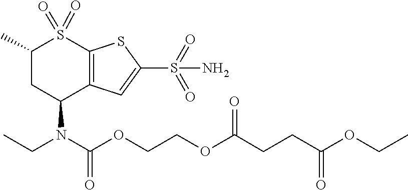 [SMILES: CCOC(=O)CCC(=O)OCCOC(=O)N(CC)[C@H]1C[C@H](C)S(=O)(=O)c2sc(S(N)(=O)=O)cc21] |
| 63-7 | 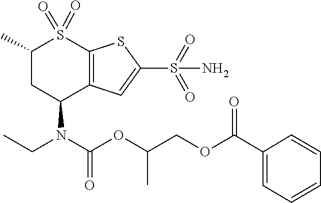 [SMILES: CCN(C(=O)OC(C)COC(=O)c1ccccc1)[C@H]1C[C@H](C)S(=O)(=O)c2sc(S(N)(=O)=O)cc21] |
| 64 | 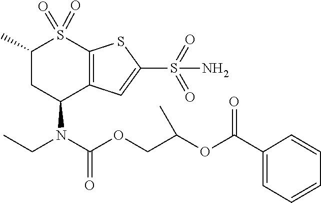 [SMILES: CCN(C(=O)OCC(C)OC(=O)c1ccccc1)[C@H]1C[C@H](C)S(=O)(=O)c2sc(S(N)(=O)=O)cc21] |
| 65-8 | 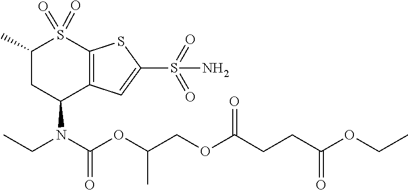 [SMILES: CCOC(=O)CCC(=O)OCC(C)OC(=O)N(CC)[C@H]1C[C@H](C)S(=O)(=O)c2sc(S(N)(=O)=O)cc21] |
| 66 | 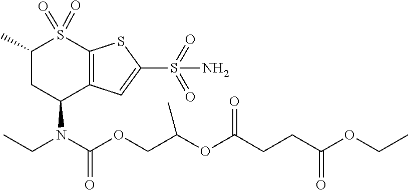 [SMILES: CCOC(=O)CCC(=O)OC(C)COC(=O)N(CC)[C@H]1C[C@H](C)S(=O)(=O)c2sc(S(N)(=O)=O)cc21] |
| 67-7 | 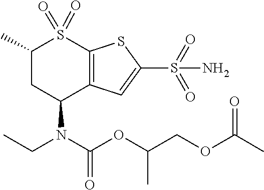 [SMILES: CCN(C(=O)OC(C)COC(C)=O)[C@H]1C[C@H](C)S(=O)(=O)c2sc(S(N)(=O)=O)cc21] |
| 68-7 | 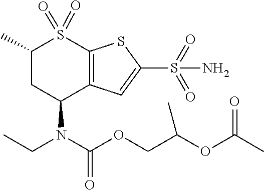 [SMILES: CCN(C(=O)OCC(C)OC(C)=O)[C@H]1C[C@H](C)S(=O)(=O)c2sc(S(N)(=O)=O)cc21] |
| 69-7 | 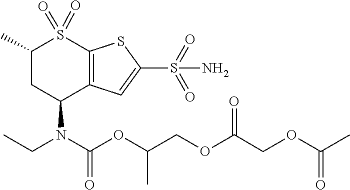 [SMILES: CCN(C(=O)OC(C)COC(=O)COC(C)=O)[C@H]1C[C@H](C)S(=O)(=O)c2sc(S(N)(=O)=O)cc21] |
| 70-7 | 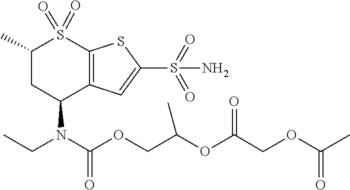 [SMILES: CCN(C(=O)OCC(C)OC(=O)COC(C)=O)[C@H]1C[C@H](C)S(=O)(=O)c2sc(S(N)(=O)=O)cc21] |
| 71-8 | 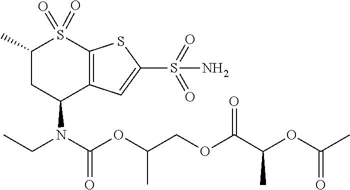 [SMILES: CCN(C(=O)OC(C)COC(=O)[C@H](C)OC(C)=O)[C@H]1C[C@H](C)S(=O)(=O)c2sc(S(N)(=O)=O)cc21] |
| 72 | 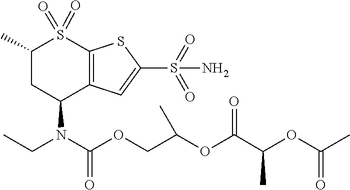 [SMILES: CCN(C(=O)OCC(C)OC(=O)[C@H](C)OC(C)=O)[C@H]1C[C@H](C)S(=O)(=O)c2sc(S(N)(=O)=O)cc21] |
| 73-9 | 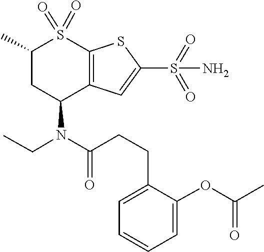 [SMILES: CCN(C(=O)CCc1ccccc1OC(C)=O)[C@H]1C[C@H](C)S(=O)(=O)c2sc(S(N)(=O)=O)cc21] |
| 74-11 | 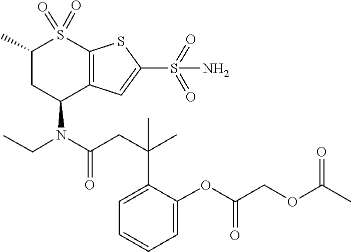 [SMILES: CCN(C(=O)CC(C)(C)c1ccccc1OC(=O)COC(C)=O)[C@H]1C[C@H](C)S(=O)(=O)c2sc(S(N)(=O)=O)cc21] |
| 75-3 | 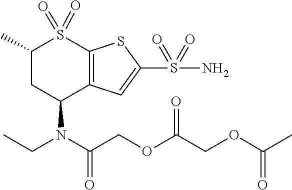 [SMILES: CCN(C(=O)COC(=O)COC(C)=O)[C@H]1C[C@H](C)S(=O)(=O)c2sc(S(N)(=O)=O)cc21] |
| 76-7 | 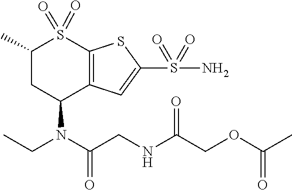 [SMILES: CCN(C(=O)CNC(=O)COC(C)=O)[C@H]1C[C@H](C)S(=O)(=O)c2sc(S(N)(=O)=O)cc21] |
| 77-7 | 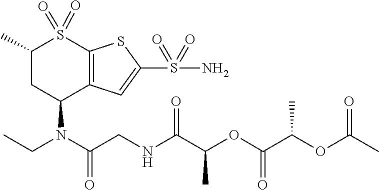 [SMILES: CCN(C(=O)CNC(=O)[C@H](C)OC(=O)[C@H](C)OC(C)=O)[C@H]1C[C@H](C)S(=O)(=O)c2sc(S(N)(=O)=O)cc21] |
| 78 | 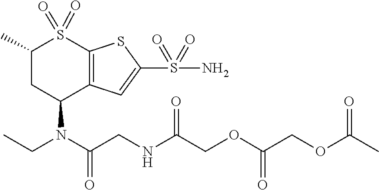 [SMILES: CCN(C(=O)CNC(=O)COC(=O)COC(C)=O)[C@H]1C[C@H](C)S(=O)(=O)c2sc(S(N)(=O)=O)cc21] |
| 79-7 | 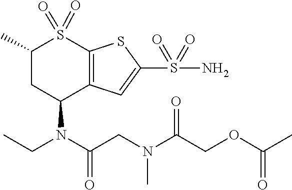 [SMILES: CCN(C(=O)CN(C)C(=O)COC(C)=O)[C@H]1C[C@H](C)S(=O)(=O)c2sc(S(N)(=O)=O)cc21] |
| 80-9 | 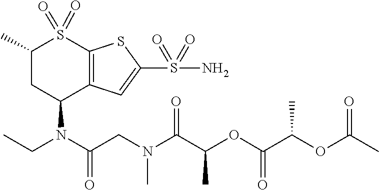 [SMILES: CCN(C(=O)CN(C)C(=O)[C@H](C)OC(=O)[C@H](C)OC(C)=O)[C@H]1C[C@H](C)S(=O)(=O)c2sc(S(N)(=O)=O)cc21] |
| 81-7 | 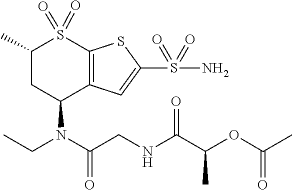 [SMILES: CCN(C(=O)CNC(=O)[C@H](C)OC(C)=O)[C@H]1C[C@H](C)S(=O)(=O)c2sc(S(N)(=O)=O)cc21] |
| 82-9 | 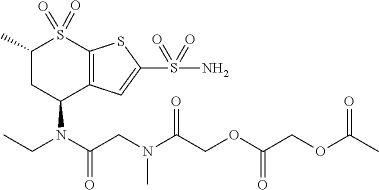 [SMILES: CCN(C(=O)CN(C)C(=O)COC(=O)COC(C)=O)[C@H]1C[C@H](C)S(=O)(=O)c2sc(S(N)(=O)=O)cc21] |
| 83-9 | 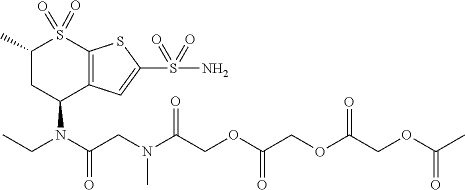 [SMILES: CCN(C(=O)CN(C)C(=O)COC(=O)COC(=O)COC(C)=O)[C@H]1C[C@H](C)S(=O)(=O)c2sc(S(N)(=O)=O)cc21] |
| 84-7 | 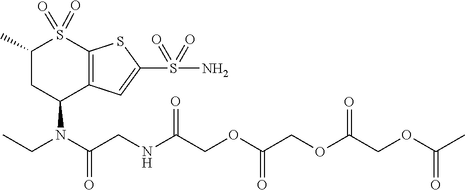 [SMILES: CCN(C(=O)CNC(=O)COC(=O)COC(=O)COC(C)=O)[C@H]1C[C@H](C)S(=O)(=O)c2sc(S(N)(=O)=O)cc21] |
| 85-9 | 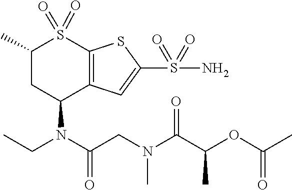 [SMILES: CCN(C(=O)CN(C)C(=O)[C@H](C)OC(C)=O)[C@H]1C[C@H](C)S(=O)(=O)c2sc(S(N)(=O)=O)cc21] |
| 86-9 | 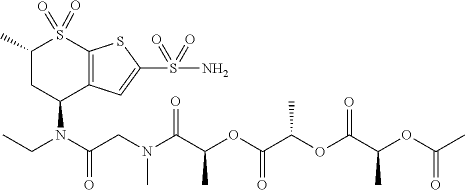 [SMILES: CCN(C(=O)CN(C)C(=O)[C@H](C)OC(=O)[C@H](C)OC(=O)[C@H](C)OC(C)=O)[C@H]1C[C@H](C)S(=O)(=O)c2sc(S(N)(=O)=O)cc21] |
| 87-7 | 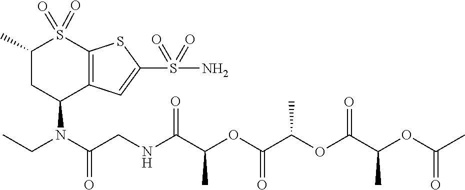 [SMILES: CCN(C(=O)CNC(=O)[C@H](C)OC(=O)[C@H](C)OC(=O)[C@H](C)OC(C)=O)[C@H]1C[C@H](C)S(=O)(=O)c2sc(S(N)(=O)=O)cc21] |
| 88-5 | 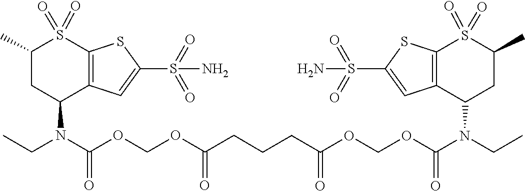 |
| 89-5 | 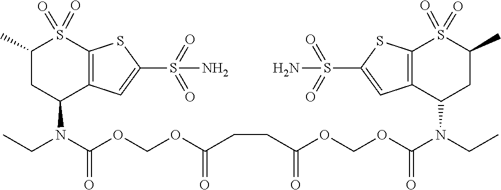 |
| 90-4 | 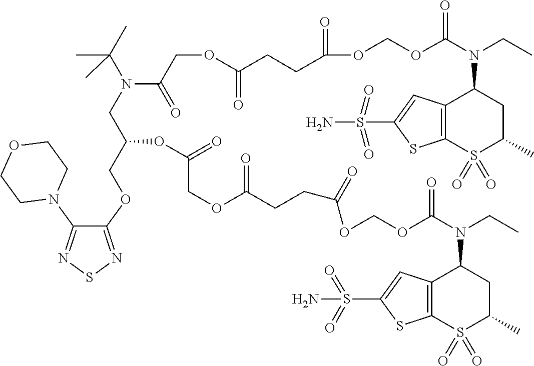 [SMILES: CCN(C(=O)OCOC(=O)CCC(=O)OCC(=O)O[C@H](COc1nsnc1N1CCOCC1)CN(C(=O)COC(=O)CCC(=O)OCOC(=O)N(CC)[C@H]1C[C@H](C)S(=O)(=O)c2sc(S(N)(=O)=O)cc21)C(C)(C)C)[C@H]1C[C@H](C)S(=O)(=O)c2sc(S(N)(=O)=O)cc21] |
| 91-4 | 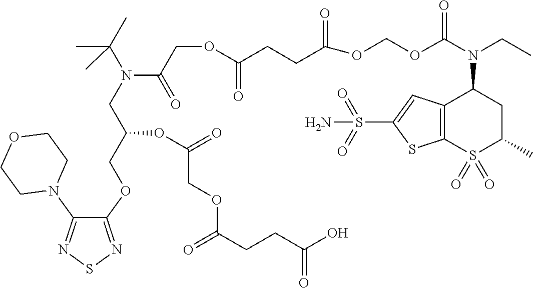 [SMILES: CCN(C(=O)OCOC(=O)CCC(=O)OCC(=O)N(C[C@@H](COc1nsnc1N1CCOCC1)OC(=O)COC(=O)CCC(=O)O)C(C)(C)C)[C@H]1C[C@H](C)S(=O)(=O)c2sc(S(N)(=O)=O)cc21] |
| 92-8 | 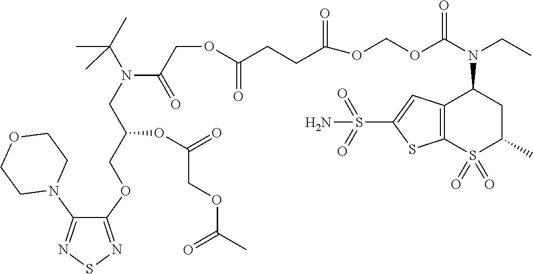 [SMILES: CCN(C(=O)OCOC(=O)CCC(=O)OCC(=O)N(C[C@@H](COc1nsnc1N1CCOCC1)OC(=O)COC(C)=O)C(C)(C)C)[C@H]1C[C@H](C)S(=O)(=O)c2sc(S(N)(=O)=O)cc21] |
| 93-4 | 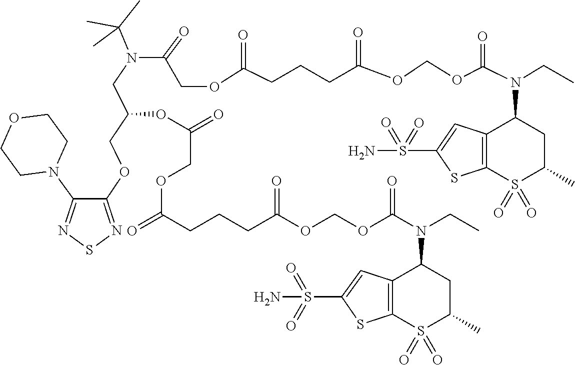 [SMILES: CCN(C(=O)OCOC(=O)CCCC(=O)OCC(=O)O[C@H](COc1nsnc1N1CCOCC1)CN(C(=O)COC(=O)CCCC(=O)OCOC(=O)N(CC)[C@H]1C[C@H](C)S(=O)(=O)c2sc(S(N)(=O)=O)cc21)C(C)(C)C)[C@H]1C[C@H](C)S(=O)(=O)c2sc(S(N)(=O)=O)cc21] |
| 93-5 | 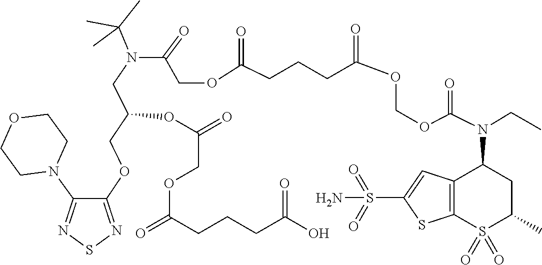 [SMILES: CCN(C(=O)OCOC(=O)CCCC(=O)OCC(=O)N(C[C@@H](COc1nsnc1N1CCOCC1)OC(=O)COC(=O)CCCC(=O)O)C(C)(C)C)[C@H]1C[C@H](C)S(=O)(=O)c2sc(S(N)(=O)=O)cc21] |
| 94 | 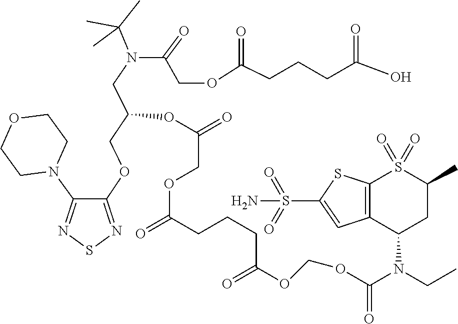 [SMILES: CCN(C(=O)OCOC(=O)CCCC(=O)OCC(=O)O[C@H](COc1nsnc1N1CCOCC1)CN(C(=O)COC(=O)CCCC(=O)O)C(C)(C)C)[C@H]1C[C@H](C)S(=O)(=O)c2sc(S(N)(=O)=O)cc21] |
| 95-5 | 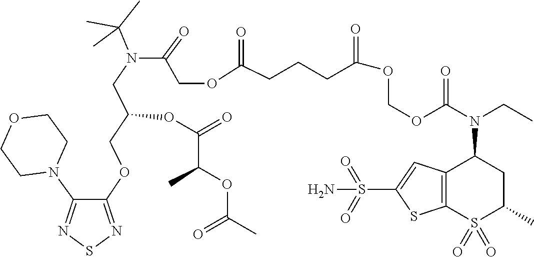 [SMILES: CCN(C(=O)OCOC(=O)CCCC(=O)OCC(=O)N(C[C@@H](COc1nsnc1N1CCOCC1)OC(=O)[C@H](C)OC(C)=O)C(C)(C)C)[C@H]1C[C@H](C)S(=O)(=O)c2sc(S(N)(=O)=O)cc21] |
| 96-5 | 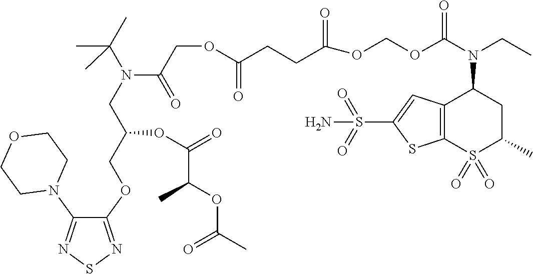 [SMILES: CCN(C(=O)OCOC(=O)CCC(=O)OCC(=O)N(C[C@@H](COc1nsnc1N1CCOCC1)OC(=O)[C@H](C)OC(C)=O)C(C)(C)C)[C@H]1C[C@H](C)S(=O)(=O)c2sc(S(N)(=O)=O)cc21] |
| 97-5 | 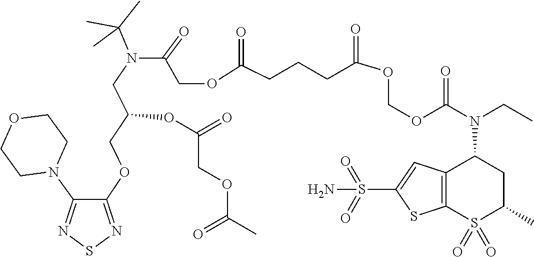 [SMILES: CCN(C(=O)OCOC(=O)CCCC(=O)OCC(=O)N(C[C@@H](COc1nsnc1N1CCOCC1)OC(=O)COC(C)=O)C(C)(C)C)[C@@H]1C[C@H](C)S(=O)(=O)c2sc(S(N)(=O)=O)cc21] |
| 98-8A | 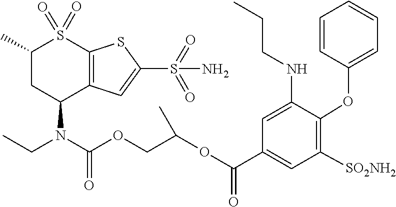 [SMILES: CCCNc1cc(C(=O)OC(C)COC(=O)N(CC)[C@H]2C[C@H](C)S(=O)(=O)c3sc(S(N)(=O)=O)cc32)cc(S(N)(=O)=O)c1Oc1ccccc1] |
| 98-8B | 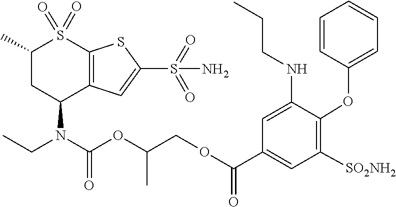 [SMILES: CCCNc1cc(C(=O)OCC(C)OC(=O)N(CC)[C@H]2C[C@H](C)S(=O)(=O)c3sc(S(N)(=O)=O)cc32)cc(S(N)(=O)=O)c1Oc1ccccc1] |
| 99-6 | 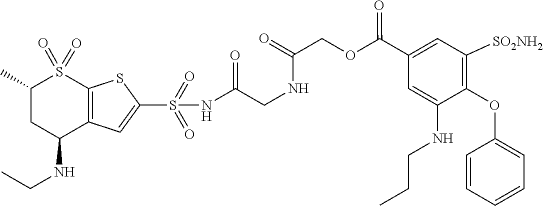 [SMILES: CCCNc1cc(C(=O)OCC(=O)NCC(=O)NS(=O)(=O)c2cc3c(s2)S(=O)(=O)[C@@H](C)C[C@@H]3NCC)cc(S(N)(=O)=O)c1Oc1ccccc1] |
| 100-11 | 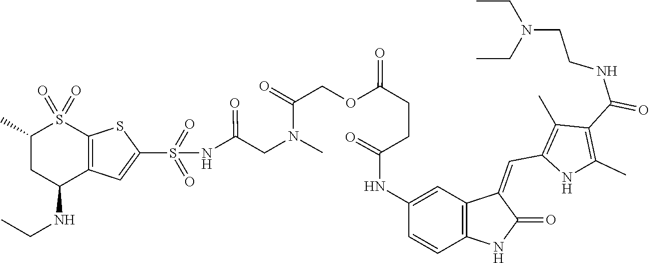 [SMILES: CCN[C@H]1C[C@H](C)S(=O)(=O)c2sc(S(=O)(=O)NC(=O)CN(C)C(=O)COC(=O)CCC(=O)Nc3ccc4c(c3)/C(=C/c3[nH]c(C)c(C(=O)NCCN(CC)CC)c3C)C(=O)N4)cc21] |
| 101-11 | 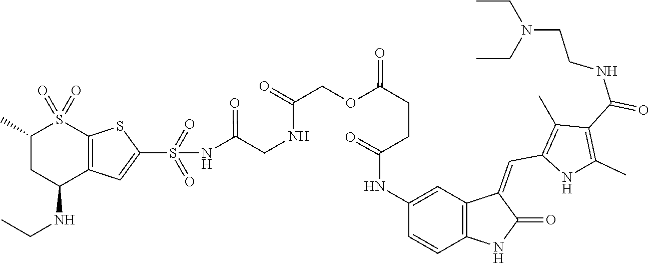 [SMILES: CCN[C@H]1C[C@H](C)S(=O)(=O)c2sc(S(=O)(=O)NC(=O)CNC(=O)COC(=O)CCC(=O)Nc3ccc4c(c3)/C(=C/c3[nH]c(C)c(C(=O)NCCN(CC)CC)c3C)C(=O)N4)cc21] |
| 102-5 | 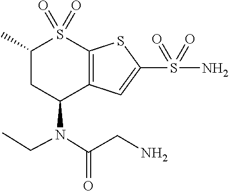 [SMILES: CCN(C(=O)CN)[C@H]1C[C@H](C)S(=O)(=O)c2sc(S(N)(=O)=O)cc21] |
| 103-7 | 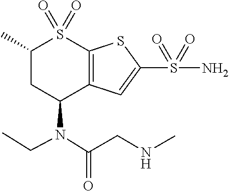 [SMILES: CCN(C(=O)CNC)[C@H]1C[C@H](C)S(=O)(=O)c2sc(S(N)(=O)=O)cc21] |
| 104-3 | 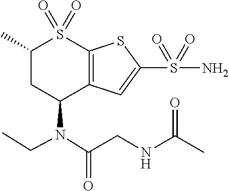 [SMILES: CCN(C(=O)CNC(C)=O)[C@H]1C[C@H](C)S(=O)(=O)c2sc(S(N)(=O)=O)cc21] |
| 105-8 | 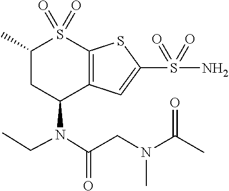 [SMILES: CCN(C(=O)CN(C)C(C)=O)[C@H]1C[C@H](C)S(=O)(=O)c2sc(S(N)(=O)=O)cc21] |
| 106 | 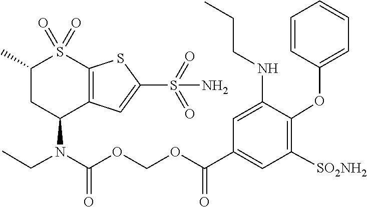 [SMILES: CCCNc1cc(C(=O)OCOC(=O)N(CC)[C@H]2C[C@H](C)S(=O)(=O)c3sc(S(N)(=O)=O)cc32)cc(S(N)(=O)=O)c1Oc1ccccc1] |
| 107 | 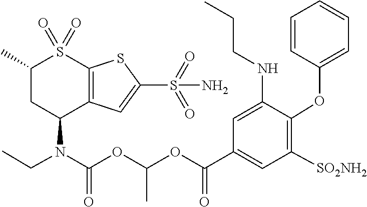 [SMILES: CCCNc1cc(C(=O)OC(C)OC(=O)N(CC)[C@H]2C[C@H](C)S(=O)(=O)c3sc(S(N)(=O)=O)cc32)cc(S(N)(=O)=O)c1Oc1ccccc1] |

### Example 8. Synthesis of Select Compounds of the Present Invention

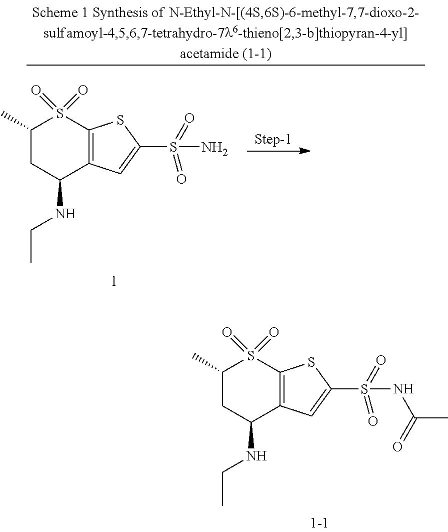 [SMILES: CCN[C@H]1C[C@H](C)S(=O)(=O)c2sc(S(N)(=O)=O)cc21 ; CCN[C@H]1C[C@H](C)S(=O)(=O)c2sc(S(=O)(=O)NC(C)=O)cc21]

To a solution of dorzolamide 1 (1.3 g, 3.61 mmol) in dichloromethane (10 V) was added triethyl amine (1.1 mL, 7.22 mmol) at 0° C. After 30 minutes, acetic acid (0.26 mL, 4.69 mmol), EDC.HCl (1.03 g, 5.41 mmol), and 4-dimethylaminopyridine (0.04 g, 0.03 mmol) were added at 0° C. The reaction mixture was allowed to stir at 25-30° C. over a period of 1 hour. The resulting reaction mass was quenched with water (100 mL), extracted with dichloromethane (250 mL×2), dried over sodium sulfate and concentrated under reduced pressure. The crude product was purified by reverse phase column chromatography to obtain product 1-1 as an off-white solid 0.35 g (34.6%). ¹H NMR (400 MHz, DMSO-d6) δ 8.7 (vbs, 2H), 7.69 (s, 1H), 4.62-4.48 (m, 1H), 3.99-3.87 (m, 1H), 3.23-3.09 (m, 1H), 3.05-2.94 (m, 1H), 2.61-2.4 (m, 2H), 1.72 (s, 3H), 1.36 (d, 3H), 1.18 (t, 3H); m/z [M+H]⁺ 367.3.

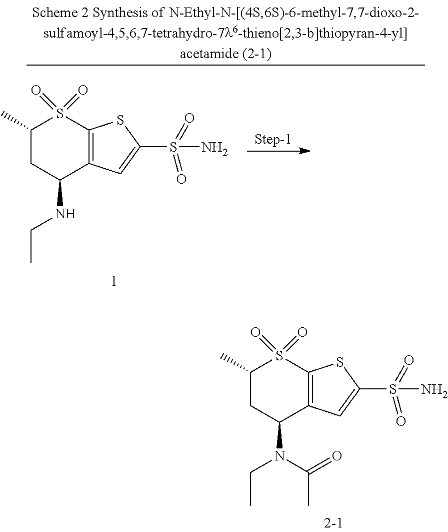 [SMILES: CCN[C@H]1C[C@H](C)S(=O)(=O)c2sc(S(N)(=O)=O)cc21 ; CCN(C(C)=O)[C@H]1C[C@H](C)S(=O)(=O)c2sc(S(N)(=O)=O)cc21]

To a solution of dorzolamide 1 (0.2 g, 0.55 mmol) in dichloromethane (10 V) was added triethyl amine (0.6 mL, 0.55 mmol) at 0° C. After 30 minutes, acetic anhydride (0.052 mL, 0.55 mmol), was added at 0° C. The reaction mixture was allowed to stir at 25-30° C. over a period of 1 hour. After completion of the reaction, the reaction was quenched with water (50 mL), extracted with ethyl acetate (100 mL), dried over sodium sulfate and concentrated under reduced pressure. The crude product was purified by reverse phase column chromatography to obtain product 2-1 as an off-white solid 0.04 g (40%). ¹H NMR (400 MHz, DMSO-d6) δ 8.05 (bs, 2H), 7.37 and 7.24 (2s, 1H), 5.3-5.1 (m, 1H), 3.98-3.87 (m, 1H), 3.49-3.37 (m, 1H), 3.31-3.18 (m, 1H), 2.82-2.72 (m, 1H), 2.43-2.31 (m, 1H), 2.22 and 2.07 (2s, 3H), 1.43 and 1.37 (2d, 3H), 1.16 and 1.01 (2t, 3H); m/z [M+H]⁺ 367.3.

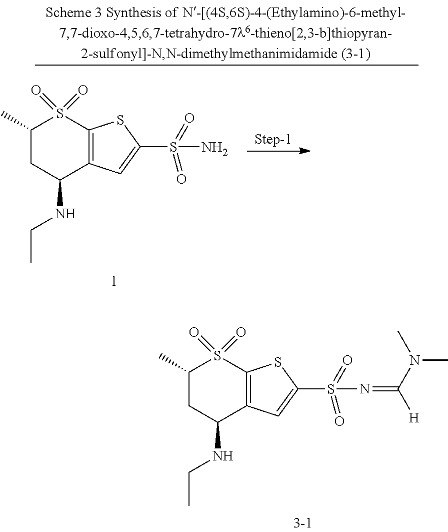

To a solution of dorzolamide 1 (0.2 g, 0.55 mmol) in N,N-dimethyl formamide (10 V) were added potassium carbonate (92 mg, 0.66 mmol), tetrabutylammonium iodide (41 mg, 0.11 mmol) and bromomethyl acetate (0.06 mL, 0.66 mmol) at 0° C. The reaction mixture was allowed to stir at 50° C. over a period of 2 hours. The crude product obtained upon evaporation of volatiles was purified by reverse phase column chromatography to afford product 3-1 as a white fluffy solid 15 mg (6.8%). ¹H NMR (400 MHz, CDCl₃) δ 8.12 (s, 1H), 7.52 (s, 1H), 3.99-3.87 (m, 2H), 3.21 (s, 3H), 3.10 (s, 3H), 2.81-2.70 (m, 2H), 2.57-2.30 (m, 2H), 1.51 (d, 3H), 1.16 (t, 3H); m/z [M+H]⁺ 380.2.

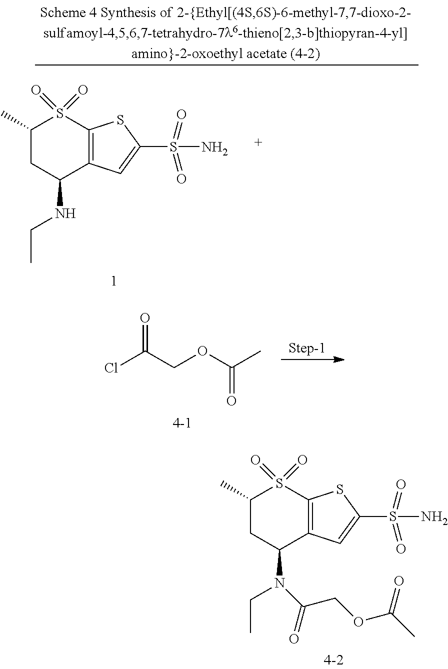

To a solution of dorzolamide 1 (1.0 g, 2.78 mmol) in dichloromethane (10 V) was added N,N-diisopropylethylamine (0.97 mL, 5.56 mmol) at 0° C. After 30 minutes, 2-chloro-2-oxoethyl acetate 4-1 (0.29 mL, 2.78 mmol) was added at 0° C. The reaction mixture was allowed to stir at 25-30° C. over a period of 1 hour. After completion of the reaction, the resulting reaction mass was quenched with water (100 mL) and extracted with ethyl acetate (300 mL), dried over sodium sulfate and concentrated under reduced pressure. The crude product obtained upon evaporation of volatiles was purified by silica gel (230-400 mesh) column chromatography (65% ethyl acetate in hexane) to obtain product 4-2 as a white solid 0.32 g (27.3%). ¹H NMR (400 MHz, DMSO-d6) δ 8.11 and 8.05 (2bs, 2H), 7.42 and 7.27 (2s, 1H), 5.22-5.00 (m, 1H), 4.88 (d, 1H), 4.76 (d, 1H), 3.97-3.86 (m, 1H), 3.5-3.1 (m, 2H), 2.86-2.60 (m, 1H), 2.45-2.30 (m, 1H), 2.11 and 2.07 (2s, 3H), 1.42 and 1.37 (2d, 3H), 1.18 and 1.00 (2t, 3H); m/z [M+H]⁺ 425.5.

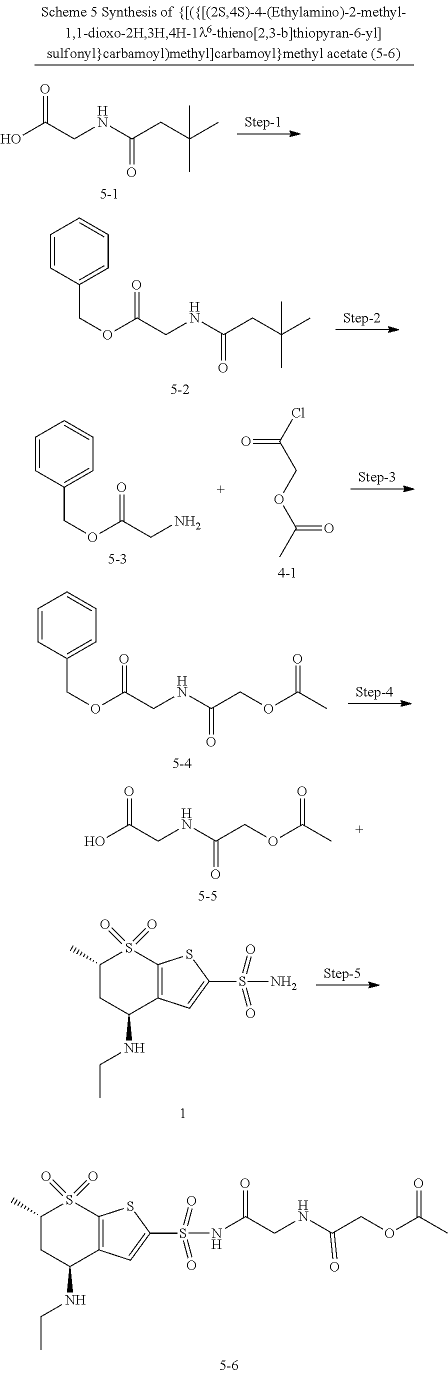

Step 1: Preparation of Benzyl [(tert-butoxycarbonyl)amino]acetate (5-2): To a solution of [(tert-butoxycarbonyl)amino]acetic acid 5-1 (5.0 g, 28.54 mmol) in dichloromethane (10 V) were added EDC.HCl (8.17 g, 42.81 mmol), benzyl alcohol (2.46 g, 22.83 mmol) and 4-dimethylaminopyridine (348 mg, 2.85 mmol) at 0° C. The reaction mixture was allowed to stir at 25-30° over a period of 1 hour. After completion of the reaction, the reaction mixture was diluted with ethyl acetate (500 mL), washed with water (200 mL), dried over sodium sulfate and concentrated under reduced pressure. The crude product obtained upon evaporation of volatiles was purified by silica gel (230-400 mesh) column chromatography (3-5% ethyl acetate in hexane) to afford product 5-2 as a colorless liquid 5.2 g (69.3%).

Step 2: Preparation of Benzyl aminoacetate (5-3): To a solution of benzyl [(tert-butoxycarbonyl)amino]acetate 5-2 (5.2 g, 19.61 mmol) in dichloromethane (10 V) was added trifluoroacetic acid (3 V) slowly at 0° C. The reaction mixture was allowed to stir at 25-30° C. over a period of 1 hour. After completion of the reaction, the resulting reaction mixture was concentrated under reduced pressure. To afford the crude compound 5-3 as a colorless liquid 5.5 g (TFA salt). The crude product 5-3 was taken forward to the next step without any further purification.

Step 3: Preparation of Benzyl [2-(acetyloxy)acetamido]acetate (5-4): To a solution of benzyl aminoacetate 5-3 (3.2 g, 19.39 mmol) in dichloromethane (10 V) was added triethyamine (7.0 mL, 48.47 mmol), 4-dimethylaminopyridine (236 mg, 1.9 mmol) and 2-chloro-2-oxoethyl acetate 4-1 (2.7 mL, 25.2 mmol) dropwise at 0° C. The reaction mixture was allowed to stir at 25-30° C. over a period of 1 hour. After completion of the reaction, the resulting reaction mass was diluted with ethyl acetate (300 mL), washed with water (100 mL), dried over sodium sulfate and concentrated under reduced pressure. The crude product obtained upon evaporation of volatiles was purified through silica gel (230-400 mesh) column (40-50% Ethyl acetate in hexane) to obtain product 5-4 as a colorless liquid 2.5 g (49%). ¹H NMR (400 MHz, DMSO-d6) δ 8.51 (t, 1H), 7.50-7.35(m, 5H), 5.13 (s, 2H), 4.53 and 4.51 (2s, 2H), 3.93 (d, 2H), 2.09 and 2.08 (2s, 3H); m/z [M+H]⁺ 266.3.

Step 4: Preparation of [2-(Acetyloxy)acetamido]acetic acid (5-5): To a 250 mL Parr shaker vessel were added a solution of benzyl [2-(acetyloxy)acetamido]acetate 5-4 (2.5 g, 9.42 mmol) in ethyl acetate (10 V) and 10% Pd/C (0.25 g, 50% wet) at 25-30° C. The reaction mixture was stirred at 25-30° C. under hydrogen pressure (5 kg/cm²) over a period of 1 hour. After completion of the reaction, the resulting reaction mixture was filtered through a celite bed and concentrated under reduced pressure to obtain product 5-5 as a white solid 1.3 g (81.2%). ¹H NMR (400 MHz, DMSO-d6) δ 12.7 (bs, 1H), 8.35 (t, 1H), 4.53 and 4.49 (2s, 2H), 3.77 (d, 2H), 2.09 and 2.08 (2s, 3H); m/z [M−H]⁻ 173.7.

Step 5: Preparation of {[({[(2S,4S)-4-(Ethylamino)-2-methyl-1,1-dioxo-2H,3H,4H-1λ⁶-thieno[2,3-b]thiopyran-6-yl]sulfonyl}carbamoyl)methyl]carbamoyl}methyl acetate (5-6): To a solution of dorzolamide 1 (2.1 g, 5.83 mmol) in dichloromethane (10 V) was added triethylamine (1.68 mL, 11.66 mmol) at 0° C. After 30 minutes, [2-(acetyloxy)acetamido]acetic acid 5-5 (1.22 g, 7.0 mmol), EDC.HCl (2.23 g, 11.66 mmol) and 4-dimethylaminopyridine (71 mg, 0.58 mmol) were added at 0° C. After completion of the reaction, the resulting reaction mixture was allowed to stir at 25-30° C. over a period of 2 hour. The resulting reaction mass was concentrated under reduced pressure. The crude was purified by reverse phase column chromatography to obtain product 5-6 as a white solid 1.0 g (35%). ¹H NMR (400 MHz, DMSO-d6) δ 8.8 (bs, 2H), 7.92 (t, 1H), 7.72 (s, 1H), 4.66-4.57 (m, 1H), 4.45 (s, 2H), 3.99-3.89 (m, 1H), 3.65-3.50 (m, 2H), 3.28-3.13 (m, 1H), 3.08-2.94 (m, 1H), 2.61-2.45 (m, 2H), 2.07 (s, 3H), 1.36 (d, 3H), 1.19 (t, 3H); m/z [M+H]⁺ 482.2.

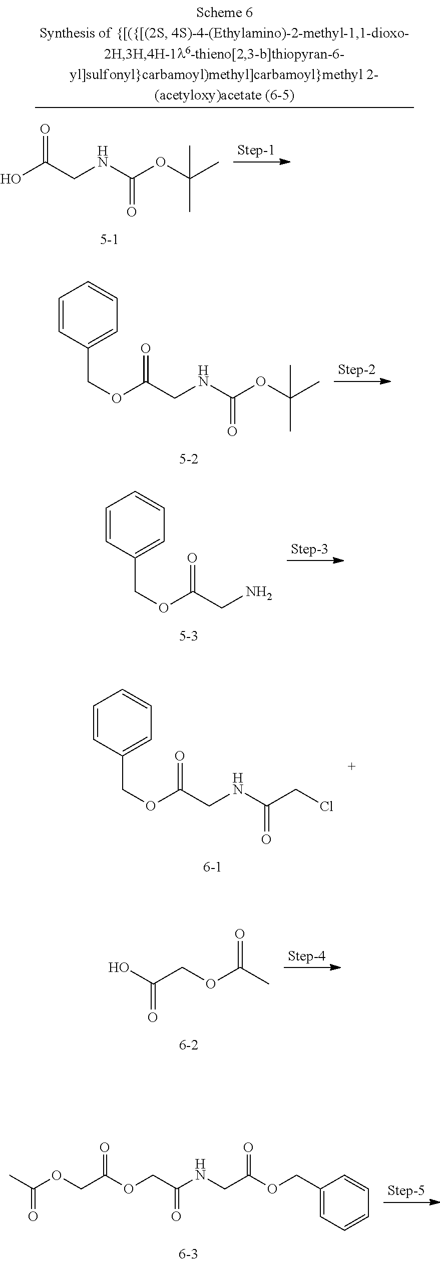

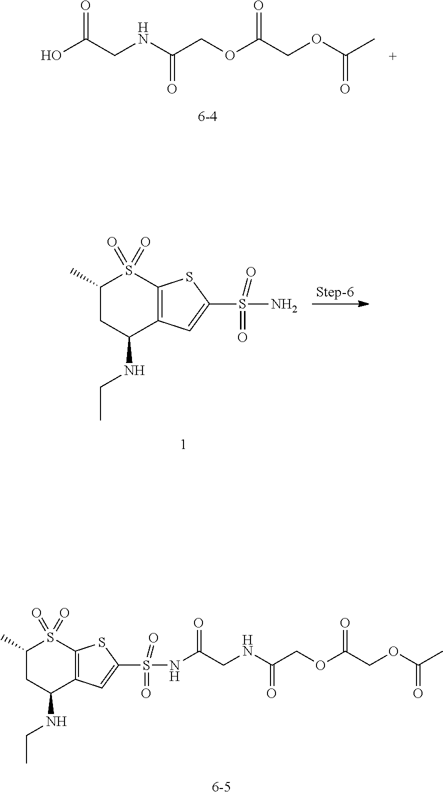

Step 1: Preparation of Benzyl [(tert-butoxycarbonyl)amino]acetate (5-2): To a solution of [(tert-butoxycarbonyl)amino]acetic acid 5-1 (5.0 g, 28.54 mmol) in dichloromethane (10 V) were added EDC.HCl (8.17 g, 42.81 mmol), benzyl alcohol (2.46 g, 22.83 mmol) and 4-dimethylaminopyridine (348 mg, 2.85 mmol) at 0° C. The reaction mixture was allowed to stir at 25-30° over a period of 1 hour. After completion of the reaction, the resulting reaction mixture was diluted with ethyl acetate (500 mL), washed with water (200 mL), dried over sodium sulfate and concentrated under reduced pressure. The crude product obtained upon evaporation of volatiles was purified through silica gel (230-400 mesh) column chromatography (3-5% ethyl acetate in hexane) to afford product 5-2 as a colorless liquid 5.2 g (69.3%).

Step 2: Preparation of Benzyl aminoacetate (5-3): To a solution of benzyl [(tert-butoxycarbonyl)amino]acetate 5-2 (5.2 g, 19.61 mmol) in dichloromethane (10 V) was added trifluoroacetic acid (3 V) slowly at 0° C. The reaction mixture was allowed to stir at 25-30° C. over a period of 1 hour. After completion of the reaction, the resulting reaction mixture was concentrated under reduced pressure. The crude compound 5-3 was obtained as a colorless liquid, 5.5 g (TFA salt). The crude product 5-3 was taken forward to the next step without any further purification.

Step 3: Preparation of Benzyl (2-chloroacetamido)acetate (6-1): To a solution of benzyl aminoacetate 5-3 (12 g, 72 mmol) in dichloromethane (10 V) were added triethylamine (26.2 mL, 181 mmol), N,N-dimethylaminopyridine (0.87 g, 7.0 mmol), and chloroacetyl chloride (7 mL, 87 mmol) at 0° C. The reaction mixture was allowed to stir at 25-30° C. over a period of 1 hour. The resulting reaction mass was quenched with water (250 mL), extracted with ethyl acetate (500 mL×2), dried over sodium sulfate and concentrated under reduced pressure. The crude product obtained upon evaporation of volatiles was purified by silica gel (230-400 mesh) column (25% ethyl acetate in hexane) to obtain product as 6-1 as an off-white solid 5.0 g (41%). ¹H NMR (400 MHz, DMSO-d6) δ 8.69 (t, 1H), 7.40-7.29 (m, 5H), 5.14 (s, 2H), 4.15 (s, 2H), 3.95 (d, 2H); m/z [M+H]⁺ 242.3.

Step 4: Preparation of {[2-(Benzyloxy)-2-oxoethyl]carbamoyl}methyl 2-(acetyloxy)acetate (6-3): To a solution of benzyl (2-chloroacetamido)acetate 6-1 (9.0 g, 37.2 mmol) in dimethylformamide (10 V) were added triethylamine (12.38 mL, 85.6 mmol), sodium iodide (6.65 g, 44.6 mmol) and acetoxyacetic acid 6-2 (5.2 g, 44 mmol) at 25-30° C. The reaction mixture was allowed to stir at 55° C. over a period of 2 hours. The resulting reaction mass was diluted with ethyl acetate (450 mL), washed with water (200 mL×2), dried over sodium sulfate and concentrated under reduced pressure. The crude product obtained upon evaporation of volatiles was purified by silica gel (230-400 mesh) column chromatography (22-25% ethyl acetate in hexane) to obtain product 6-3 as a colorless wax 6.0 g (50%). ¹H NMR (400 MHz, DMSO-d6) δ 8.39 (t, 1H), 7.47-7.28 (m, 5H), 5.14 (s, 2H), 4.77 (s, 2H), 4.62 (s, 2H), 3.95 (d, 2H), 2.11 (s, 3H); m/z [M+H]⁺ 324.3.

Step 5: Preparation of 2-(2-{[2-(Acetyloxy)acetyl]oxy}acetamido)acetic acid (6-4): To a 250 mL Parr shaker vessel were added a solution {[2-(benzyloxy)-2-oxoethyl] carbamoyl} methyl 2-(acetyloxy)acetate 6-3 (3.0 g, 9.28 mmol) in ethyl acetate (10 V) and 10% Pd/C (0.3 g, 50% wet) at 25-30° C. The reaction mixture was stirred at 25-30° C. under hydrogen pressure (5 kg/cm²) over a period of 1 hour. After completion of the reaction, the resulting reaction mixture was filtered through a celite bed and concentrated under reduced pressure to obtain product 6-4 as a waxy solid 1.9 g (90%). ¹H NMR (400 MHz, DMSO-d6) δ 12.7 (bs, 1H), 8.39 (t, 1H), 4.76 (s, 2H), 4.60 (s, 2H), 3.79 (d, 2H), 2.11 (s, 3H); m/z [M+H]⁺ 234.1.

Step 6: Preparation of {[({[(2S,4S)-4-(Ethylamino)-2-methyl-1,1-dioxo-2H,3H,4H-1λ⁶-thieno[2,3-b]thiopyran-6-yl]sulfonyl}carbamoyl)methyl]carbamoyl}methyl 2-(acetyloxy)acetate (6-5): To a solution dorzolamide 1 (1.6 g, 4.4 mmol) in dichloromethane (10 V) were added N,N-diisopropylethylamine (1.22 mL, 6.6 mmol), EDC.HCl (1.27 g, 6.6 mmol), 2-(2-{[2-(acetyloxy) acetyl]oxy}acetamido)acetic acid 6-4 (1.34 g, 5.47 mmol) and N,N-dimethylamino pyridine (54 mg, 0.4 mmol) 0° C. The reaction mixture was allowed to stir for 2 hours at 25-30° C. After completion of reaction, the resulting reaction mass was concentrated under reduced pressure. The crude product was purified by reverse phase column chromatography to obtain product 6-5 as a white solid 0.51 g (19%). ¹H NMR (400 MHz, DMSO-d6) δ 8.8 (bs, 2H), 7.99 (t, 1H), 7.71 (s, 1H), 4.74 (s, 2H), 4.66-4.51 (m, 3H), 4.00-3.88 (m, 1H), 3.68-3.48 (m, 2H), 3.28-3.12 (m, 1H), 3.10-2.92 (m, 1H), 2.65-2.45 (m, 2H), 2.11 (s, 3H), 1.36 (d, 3H), 1.23 (t, 3H); m/z [M+H]⁺ 540.3.

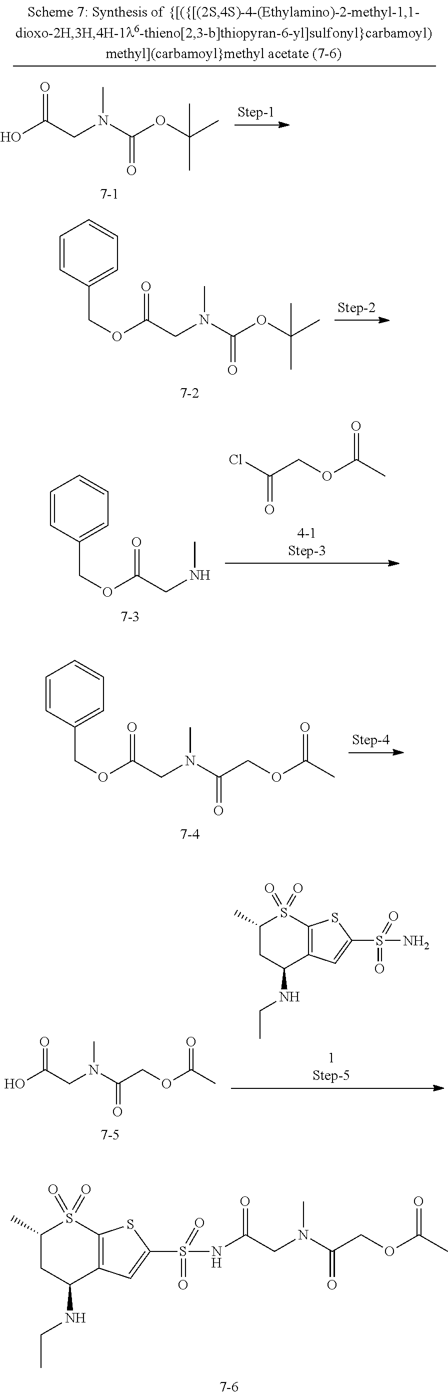

Step 1: Preparation of Benzyl [(tert-butoxycarbonyl)(methyl)amino]acetate (7-2): To a solution of [(tert-butoxycarbonyl)(methyl)amino]acetic acid 7-1 (25.0 g, 132.2 mmol) in dichloromethane (10 V) were added EDC.HCl (37.89 g, 198.4 mmol), benzyl alcohol (11.44 g, 105.81 mmol) and 4-dimethylaminopyridine (1.61 g, 13.2 mmol) at 0° C. The reaction mixture was allowed to stir at 25-30° C. over a period of 1 hour. After completion of the reaction, the resulting reaction mixture was diluted with ethyl acetate (1 L), washed with water (500 mL), dried over sodium sulfate and concentrated under reduced pressure. The crude product obtained upon evaporation of volatiles was purified through silica gel (230-400 mesh) column chromatography (3-4% ethyl acetate in hexane) to give product 7-2 as a colorless liquid 26.0 g (72.2%).

Step 2: Preparation of Benzyl (methylamino)acetate (7-3): To a solution of benzyl [(tert-butoxycarbonyl)(methyl)amino]acetate 7-2 (20.0 g, 71.62 mmol) in dichloromethane (10 V) was added trifluoroacetic acid slowly at 0° C. The reaction mixture was allowed to stir at 25-30° C. over a period of 1 hours. After completion of the reaction, the resulting reaction mixture was concentrated under reduced pressure to obtain the crude compound 7-3 as a colorless liquid, 22.0 g, as a TFA salt. The crude product 7-3 was taken forward to the next step without any further purification.

Step 3: Preparation of Benzyl {[(acetyloxy)acetyl](methyl)amino}acetate (7-4): To a solution of benzyl (methylamino)acetate 7-3 (13.0 g, 72.62 mmol) in dichloromethane (10 V) were added triethyl amine (26.24 mL, 181.55 mmol), 4-dimethylamino pyridine (0.88 g, 7.26 mmol) and 2-chloro-2-oxoethyl acetate 4-1 (10.15 mL, 94.41 mmol) slowly at 0° C. The reaction mixture was allowed to stir at 25-30° C. over a period of 1 hour. After completion of the reaction, the resulting reaction mass was diluted with ethyl acetate (500 mL), washed with water (200 ×2 mL), dried over sodium sulfate and concentrated under reduced pressure. The crude product obtained upon evaporation of volatiles was purified through silica gel (230-400 mesh) column chromatography (40% ethyl acetate in hexane) to obtain product 7-4 as a colorless liquid 12.0 g (59.17%).

Step 4: Preparation of {[(Acetyloxy)acetyl](methyl)amino}acetic acid (7-5): To a 250 mL Parr shaker vessel was added a solution of benzyl{[(acetyloxy)acetyl](methyl)amino}acetate 7-4 (12.0 g, 42.96 mmol) in ethyl acetate (10 V) and 10% Pd/C (1.2 g, 50% wet) at 25-30° C. The reaction mixture was stirred at 25-30° C. under hydrogen pressure (5 kg/cm²) over a period of 1 hour. After completion of the reaction, the resulting reaction mixture was filtered through celite bed and concentrated under reduced pressure to obtain product 7-5 as a white solid 7.0 g (86.1%).

Step 5: Preparation of {[({[(2S,4S)-4-(Ethylamino)-2-methyl-1,1-dioxo-2H,3H,4H-1λ⁶-thieno[2,3-b]thiopyran-6-yl]sulfonyl}carbamoyl)methyl](methyl)carbamoyl}methyl acetate (7-6): To a solution of dorzolamide 1 (1.0 g, 2.77 mmol) in dichloromethane (10 V) was added triethyl amine (0.8 mL, 5.54 mmol) at 0° C. After 30 minutes, {[(acetyloxy)acetyl](methyl) amino} acetic acid 7-5 (0.63 g, 3.33 mmol), EDC.HCl (0.74 g, 3.87 mmol) and 4-dimethylaminopyridine (0.033 g, 0.27 mmol) were added at 0° C. After completion of the reaction, the resulting reaction mixture was allowed to stir at 25-30° C. over a period of 1 hour. The resulting reaction mass was concentrated under reduced pressure. The crude product obtained upon evaporation was purified by reverse phase column chromatography to obtain product 7-6 as a white solid 1.0 g (72.7%). ¹H NMR (400 MHz, DMSO-d6) δ 8.8 (bs, 2H), 7.74 and 7.70 (2s, 1H), 4.78-4.56 (m, 3H), 4.01-3.89 (m, 1H), 3.84-3.67 (m, 2H), 3.27-3.14 (m, 1H), 3.05-2.94 (m, 1H), 2.88 and 2.74 (2s, 3H), 2.61-2.45 (m, 2H), 2.04 (s, 3H), 1.35 (d, 3H), 1.18 (t, 3H); m/z [M+H]⁺ 496.3.

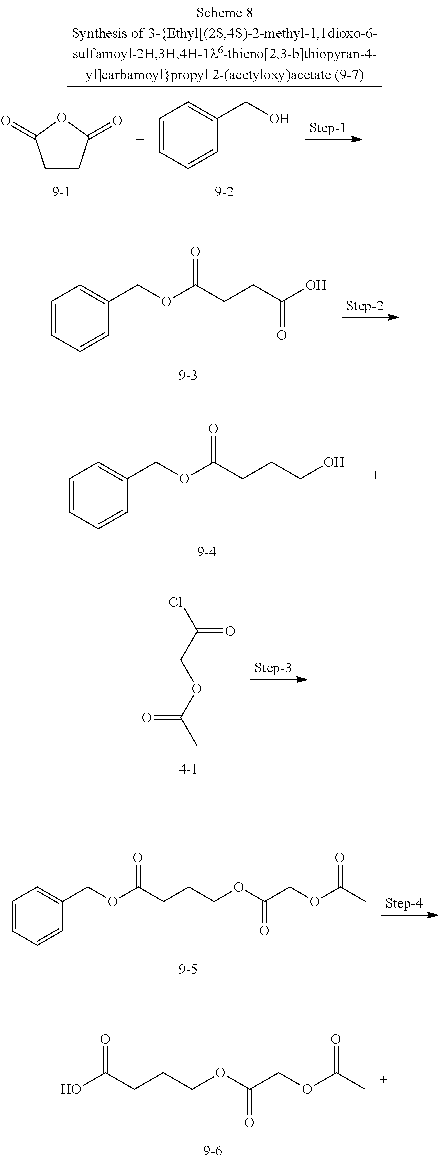

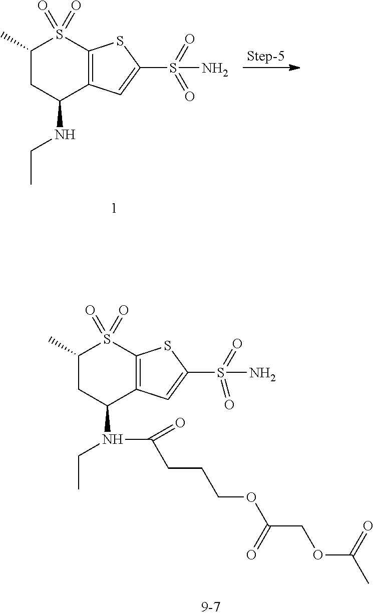 [SMILES: CCN[C@H]1C[C@H](C)S(=O)(=O)c2sc(S(N)(=O)=O)cc21 ; CCN(C(=O)CCCOC(=O)COC(C)=O)[C@H]1C[C@H](C)S(=O)(=O)c2sc(S(N)(=O)=O)cc21]

Step 1: Preparation of 4-(Benzyloxy)-4-oxobutanoic acid (9-3): To a solution of benzyl alcohol 9-2 (5.92 g, 54.85 mmol) in dichloromethane (10 V) were added triethylamine (7.71 mL, 54.85 mmol), oxolane-2,5-dione 9-1 (5.0 g, 49.86 mmol) and 4-dimethylaminopyridine (61 mg, 0.49 mmol) at 0° C. The reaction mixture was allowed to stir at 25-30° C. over a period of 10 hours. After completion of the reaction, the resulting reaction mixture was diluted with dichloromethane (200 mL) and washed with 5% NaHCO3 solution (100 mL). The aqueous layer was separated, acidified with 1.5N HCl and extracted with ethyl acetate (300 mL). The organic layer was dried over sodium sulfate and concentrated under reduced pressure to obtain product 9-3 as a white solid 8.0 g (77%). ¹H NMR (400 MHz, DMSO-d6) δ 12.24 (s, 1H), 7.41-7.27 (m, 5H), 5.90 (s, 2H), 2.6-2.4 (m, 4H); m/z [M+H]⁺ 209.2.

Step 2: Preparation of Benzyl 4-hydroxybutanoate (9-4): To a solution of 4-(benzyloxy)-4-oxobutanoic acid 9-3 (20.0 g, 96.05 mmol) in tetrahydrofuran (10 V) was added borane-dimethyl sulfide (61.72 mL, 124.86 mmol) at ⁻10-5° C. The reaction mixture was allowed to stir at this temperature for 1 hour and then allowed to stir at 25-30° C. for 6 hours. After completion of the reaction, the resulting reaction mixture was cooled to 0° C., quenched with saturated potassium carbonate solution (300 ml), then extracted with ethyl acetate (500×3 mL). The organic extract was dried over sodium sulfate and concentrated under reduced pressure. The crude product obtained upon evaporation of volatiles was purified by silica gel (60-120 mesh) column chromatography (30% Ethyl acetate in hexane) to obtain product 9-4 as a colorless oil 16.0 g (85%).

Step 3: Preparation of Benzyl 4-{[(acetyloxy)acetyl]oxy}butanoate (9-5): To a solution of benzyl 4-hydroxybutanoate 9-4 (2.0 g, 10.31 mmol) in dichloromethane (10 V) were added triethyl amine (3.58 mL, 24.74 mmol), 4-dimethylamino pyridine (0.25 g, 2.06 mmol) and 2-chloro-2-oxoethyl acetate 4-1 (1.68 g, 12.37 mmol) slowly at 0° C. The reaction mixture was allowed to stir at 25-30° C. over a period of 1 hour. After completion of the reaction, the resulting reaction mass was quenched with water (100 mL), extracted with ethyl acetate (200 mL), dried over sodium sulfate and concentrated under reduced pressure. The crude product obtained upon evaporation of volatiles was purified through silica gel (60-120 mesh) column (40% ethyl acetate in hexane) to obtain product 9-5 as a colorless liquid 2.0 g (66%). ¹H NMR (400 MHz, DMSO-d6) δ 7.40-7.26 (m, 5H), 5.10 (s, 2H), 4.62 (s, 2H), 4.12 (t, 2H), 2.44 (t, 2H), 1.90-1.80 (m, 2H); m/z [M+H]⁺ 295.1.

Step 4: Preparation of 4-{[(Acetyloxy)acetyl]oxy}butanoic acid (9-6): To a 250 mL Parr shaker vessel was added a solution of benzyl 4-{[(acetyloxy)acetyl]oxy}butanoate 9-5 (1.5 g, 5.09 mmol) in ethyl acetate (10 V) and 10% Pd/C (0.15 g, 50% wet) at 25-30° C. The reaction mixture was stirred at 25-30° C. under hydrogen pressure (5 kg/cm²) over a period of 1 hour. After completion of the reaction, the resulting reaction mixture was filtered through a celite bed and concentrated under reduced pressure to obtain product 9-6 as a waxy solid 0.9 g (86.5%). ¹H NMR (400 MHz, DMSO-d6) δ 12.2 (bs, 1H), 4.64 (s, 2H), 4.10 (t, 2H), 2.28 (t, 2H), 1.84-1.73 (m, 2H); m/z [M+H]⁺ 205.1.

Step 5: Preparation of 3-{Ethyl[(2S,4S)-2-methyl-1,1-dioxo-6-sulfamoyl-2H,3H,4H-1λ⁶-thieno[2,3-b]thiopyran-4-yl]carbamoyl}propyl 2-(acetyloxy)acetate (9-7): To a solution of 4-{[(acetyloxy)acetyl]oxy}butanoic acid 9-6 (0.88 g, 4.33 mmol) in dichloromethane (10 V), were added oxalyl chloride (0.51 mL, 5.99 mmol) and N,N-dimethylformamide (0.12 mL) at 0° C. The reaction mixture was allowed to stir at 25-30° C. over a period of 30 minutes. After completion of the reaction, the resulting reaction mass was concentrated under reduced pressure under inert atmosphere. The crude material obtained was dissolved in dichloromethane (5V) and added to a solution of dorzolamide 1 (1.2 g, 3.33 mmol) and N,N-diisopropylethylamine (1.45 mL, 8.32 mmol) in dichloromethane (5V) at 0° C. The reaction mixture was allowed to stir at 25-30° C. over a period of 1 hour. After completion of the reaction, the resulting reaction mass was quenched with water (100 mL), extracted with ethyl acetate (300 mL), dried over sodium sulfate and concentrated under reduced pressure. The crude product obtained upon evaporation of volatiles was purified by silica gel (230-400 mesh) column chromatography (60-70% ethyl acetate in hexane) to obtain product 9-7 as a waxy solid 0.7 g (41%). ¹H NMR (400 MHz, DMSO-d6) δ 8.08 and 8.03 (2bs, 2H), 7.38 and 7.23 (2s, 1H), 5.34-5.07 (m, 1H), 4.64 (s, 2H), 4.20-4.07 (m, 2H), 3.97-3.86 (m, 1H), 3.5-3.1 (m, 2H), 2.86-2.64 (m, 1H), 2.5-2.24 (m, 3H), 2.10 and 2.08 (2s, 3H), 1.92-1.74 (m, 2H), 1.43 and 1.37 (2d, 3H), 1.15 and 1.01 (2t, 3H); m/z [M+H]⁺ 511.4.

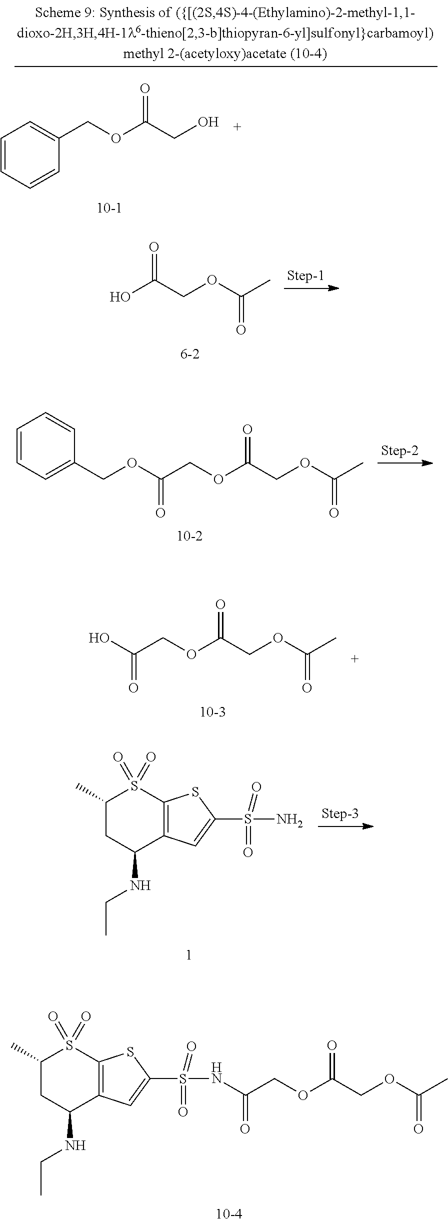

Step 1: Preparation of 2-(Benzyloxy)-2-oxoethyl (acetyloxy)acetate (10-2): To a solution of (acetyloxy)acetic acid 6-2 (4.97 g, 42.13 mmol) in dichloromethane (10 V) were added EDC.HCl (9.77 g, 51.15 mmol), benzyl hydroxyacetate 10-1 (5.0 g, 30.09 mmol) and 4-dimethylaminopyridine (367 mg, 3.01 mmol) at 0° C. The reaction mixture was allowed to stir at 25-30° C. over a period of 1 hour. After completion of the reaction, the resulting reaction mixture was diluted with ethyl acetate (300 mL), washed with water (150 mL), dried over sodium sulfate and concentrated under reduced pressure. The crude product obtained upon evaporation of volatiles was purified through silica gel (230-400 mesh) column chromatography (8% ethyl acetate in hexane) to afford product 10-2 as a colorless liquid 6.5 g (81%). ¹H NMR (400 MHz, DMSO-d6) δ 7.41-7.29 (m, 5H), 5.18 (s, 2H), 4.83 (s, 2H), 4.77 (s, 2H), 2.10 (s, 3H); m/z [M+H]⁺ 267.2, [M+NH₄]⁺ 284.4, [M+Na]⁺ 289.3.

Step 2: Preparation of {[(Acetyloxy)acetyl]oxy}acetic acid (10-3): To a 250 mL Parr shaker vessel were added a solution of 2-(benzyloxy)-2-oxoethyl (acetyloxy)acetate 10-2 (6.5 g, 24.4 mmol) in ethyl acetate (10 V) and 10% Pd/C (0.65 g, 50% wet) at 25-30° C. The reaction mixture was stirred at room 25-30° C. under hydrogen pressure (5 kg/cm²) over a period of 1 hour. After completion of the reaction, the resulting reaction mixture was filtered through a celite bed and concentrated under reduced pressure to obtain product 10-3 as a waxy solid 4.0 g (93.2%). ¹H NMR (400 MHz, DMSO-d6) δ 13.2 (bs, H), 4.74 (s, 2H), 4.65 (s, 2H), 2.10 (s, 3H); m/z [M+H]⁺ 177.2, [M+NH₄]⁺ 194.2, [M+Na]⁺ 199.1.

Step 3: Preparation of ({[(2S,4S)-4-(Ethylamino)-2-methyl-1,1-dioxo-2H,3H,4H-1λ⁶-thieno[2,3-b]thiopyran-6-yl]sulfonyl}carbamoyl)methyl 2-(acetyloxy)acetate (10-4): To a solution of {[(acetyloxy)acetyl]oxy}acetic acid 10-3 (2.45 g, 13.9 mmol) in dichloromethane (10 V) were added oxalyl chloride (1.43 mL, 16.68 mmol) and N,N-dimethylformamide (0.2 mL) at 0° C. The reaction mixture was allowed to stir at 25-30° C. over a period of 30 minutes. After completion of the reaction, the resulting reaction mass was concentrated under reduced pressure under inert atmosphere. The crude obtained was dissolved in dichloromethane (5V) and added to a solution of dorzolamide 1 (2 g, 5.56 mmol), N,N-diisopropylethylamine (1.93 mL, 11.1 mmol) in dichloromethane (5 V) at 0° C. and 4-dimethylaminopyridine (68 mg, 0.56 mmol) at 0° C. The reaction mixture was allowed to stir at 25-30° C. over a period of 1 hour. After completion of the reaction, the resulting reaction mass was quenched with water (100 mL), extracted with dichloromethane (300 mL), dried over sodium sulfate and concentrated under reduced pressure. The crude product obtained upon evaporation of volatiles was purified by reverse phase column chromatography to obtain product 10-4 as a white solid 1.0 g (37%).

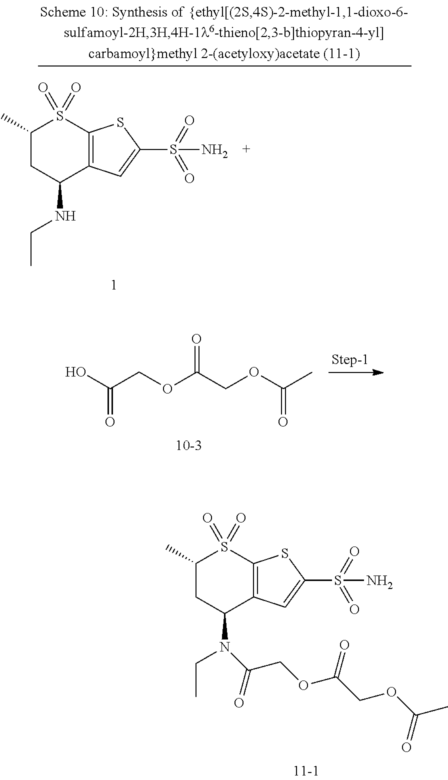 [SMILES: CCN[C@H]1C[C@H](C)S(=O)(=O)c2sc(S(N)(=O)=O)cc21 ; CC(=O)OCC(=O)OCC(=O)O ; CCN(C(=O)COC(=O)COC(C)=O)[C@H]1C[C@H](C)S(=O)(=O)c2sc(S(N)(=O)=O)cc21]

Step 1: Preparation of {Ethyl[(2S,4S)-2-methyl-1,1-dioxo-6-sulfamoyl-2H,3H,4H-1λ⁶-thieno[2,3-b]thiopyran-4-yl]carbamoyl}methyl 2-(acetyloxy)acetate (11-1): To a solution of {[(acetyloxy)acetyl]oxy}acetic acid 10-3 (1.1 g, 6.25 mmol) in dichloromethane (10 V) were added oxalyl chloride (0.71 mL, 8.34 mmol) and N,N-dimethylformamide (0.15 mL) at 0° C. The reaction mixture was allowed to stir at 25-30° C. over a period of 30 minutes. After completion of the reaction, the resulting reaction mass was concentrated under reduced pressure and inert atmosphere. The crude obtained was dissolved in dichloromethane (5V) and added to a solution of dorzolamide 1 (1.5 g, 4.17 mmol), N,N-diisopropylethylamine (0.79 mL, 8.34 mmol) in dichloromethane (5 V) at 0° C. The reaction mixture was allowed to stir at 25-30° C. over a period of 1 hour. After completion of the reaction, the resulting reaction mass was quenched with water (100 mL), extracted with ethyl acetate (300 mL). The organic extracts were dried over sodium sulfate, filtered and concentrated under reduced pressure. The crude product obtained upon evaporation of volatiles was purified by silica gel (230-400 mesh) column (75% ethyl acetate in hexane) to obtain product 11-4 as white solid 0.2 g (10%).

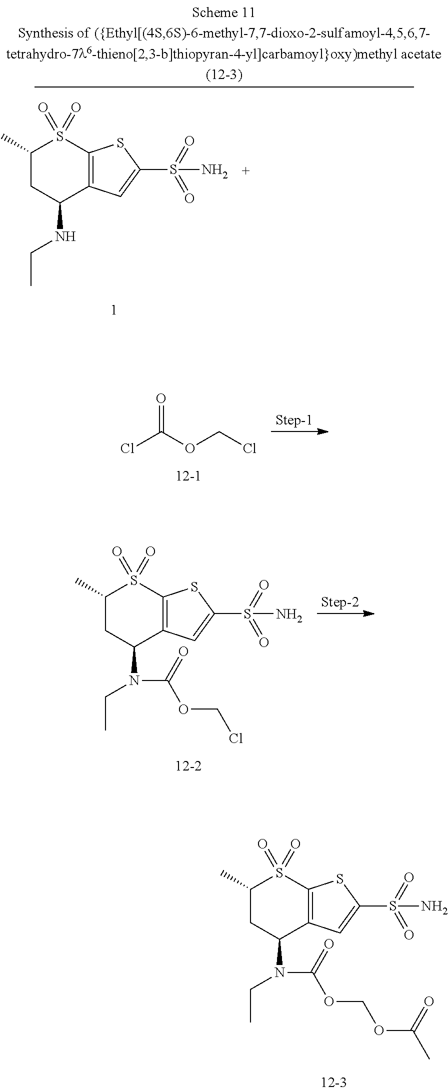 [SMILES: CCN[C@H]1C[C@H](C)S(=O)(=O)c2sc(S(N)(=O)=O)cc21 ; O=C(Cl)OCCl ; CCN(C(=O)OCCl)[C@H]1C[C@H](C)S(=O)(=O)c2sc(S(N)(=O)=O)cc21 ; CCN(C(=O)OCOC(C)=O)[C@H]1C[C@H](C)S(=O)(=O)c2sc(S(N)(=O)=O)cc21]

Step 1: Preparation of Chloromethyl ethyl[(4S,6S)-6-methyl-7,7-dioxo-2-sulfamoyl-4,5,6,7-tetrahydro-7λ⁶-thieno[2,3-b]thiopyran-4-yl]carbamate (12-2): To a solution of dorzolamide 1 (4 g, 12.3 mmol) in dichloromethane (10 V) was added N,N-diisopropylethylamine (4.07 mL, 24.6 mmol) at 0° C. After 30 minutes, chloromethyl chloroformate 12-1 (2.1 mL, 22.7 mmol) was added at 0° C. The reaction mixture was allowed to stir at 25-30° C. over a period of 1 hour. The resulting reaction mass was quenched with water (80 mL), extracted with ethyl acetate (150 mL×2), and dried over sodium sulfate and concentrated under reduced pressure. The crude product obtained upon evaporation of volatiles was purified by silica gel (230-400 mesh) column chromatography (50% ethyl acetate in hexane) to obtain product 12-2 as an off-white solid 2.7 g (57%).

Step 2: Preparation of ({Ethyl[(4S,6S)-6-methyl-7,7-dioxo-2-sulfamoyl-4,5,6,7-tetrahydro-7λ⁶-thieno[2,3-b]thiopyran-4-yl]carbamoyl}oxy)methyl acetate (12-3): To a solution of chloromethyl ethyl[(4S,6S)-6-methyl-7,7-dioxo-2-sulfamoyl-4,5,6,7-tetrahydro-7λ⁶-thieno[2,3-b]thiopyran-4-yl]carbamate 12-2 (1.3 g, 3.3 mmol) in N,N-dimethyl formamide (10 V) were added triethyl amine (0.87 mL, 6.7 mmol), sodium iodide (0.759 g, 5.0 mmol) and acetic acid (0.30 mL, 5.0 mmol) at 25-30° C. The reaction mixture was allowed to stir at 55° C. over a period of 2 hours. The resulting reaction mass was diluted with ethyl acetate (150 mL) and the organic extract was washed with water (100 mL×2), dried over sodium sulfate and concentrated under reduced pressure. The crude product obtained upon evaporation of volatiles was purified by silica gel (230-400 mesh) column (60% ethyl acetate in hexane) to obtain product 12-3 as off-white solid 0.6 g (42%). ¹H NMR (400 MHz, DMSO-d6) δ 8.16 (bs, 2H), 7.30 (s, 1H), 5.71-5.46 (m, 2H), 5.13-4.94 (m, 1H), 3.98-3.79 (m, 1H), 3.4-3.1 (m, 2H), 2.85-2.74 (m, 1H), 2.5-2.4 (m, 1H), 2.1-2.0 (m, 3H), 1.4-1.3 (m, 3H) 1.2-1.05 (m, 3H); m/z [M−H]⁻ 439.0.

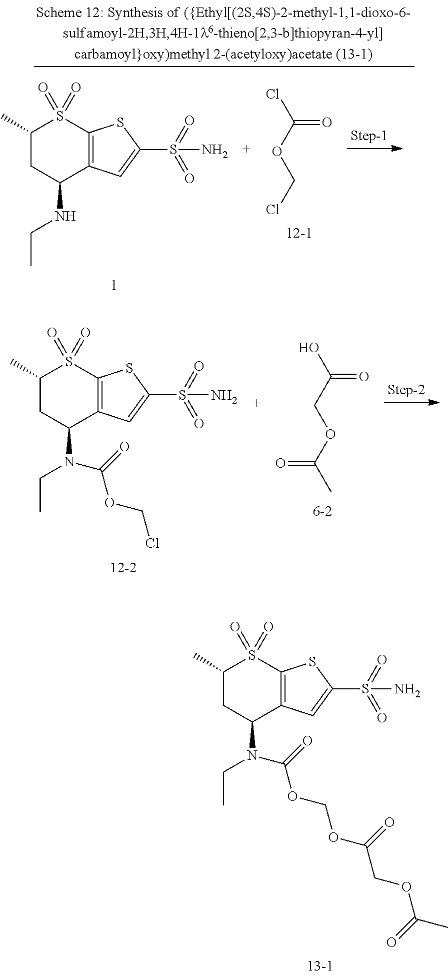

Step 1: Preparation of ({Ethyl[(2S,4S)-2-methyl-1,1-dioxo-6-sulfamoyl-2H-3H,4H-1λ⁶-thieno[2,3-b]thiopyran-4-yl]carbamoyl}oxy)methyl 2-(acetyloxy)acetate (13-1): To a solution of chloromethyl ethyl[(4S,6S)-6-methyl-7,7-dioxo-2-sulfamoyl-4,5,6,7-tetrahydro-7λ⁶-thieno[2,3-b]thiopyran-4-yl]carbamate 12-2 (1.4 g, 3.6 mmol) in N,N-dimethyl formamide (10 V) were added triethylamine (0.94 mL, 7.2 mmol), sodium iodide (0.81 g, 5.0 mmol) and acetoxy acetic acid 6-2 (0.64 mL, 5.0 mmol) at 25-30° C. The reaction mixture was allowed to stir at 55° C. over a period of 2 hours. The resulting reaction mass was diluted with ethyl acetate (150 mL), washed with water (100 mL×2), dried over sodium sulfate and concentrated under reduced pressure. The crude product obtained upon evaporation of volatiles was purified through silica gel (230-400 mesh) column chromatography (50% ethyl acetate in hexane) to obtain product 13-1 as an off-white solid 0.43 g (24%). ¹H NMR (400 MHz, DMSO-d6) δ 8.06 (bs, 2H), 7.36-7.27 (m, 1H), 5.77-5.59 (m, 2H), 5.12-5.02 (m, 1H), 4.72-4.63 (m, 2H), 3.97-3.79 (m, 1H), 3.4-3.1 (m, 2H), 2.85-2.70 (m, 1H), 2.5-2.4 (m, 1H), 2.10 (s, 3H), 1.41-1.33 (m, 3H) 1.11 (t, 3H); m/z [M−H]⁻ 497.0.

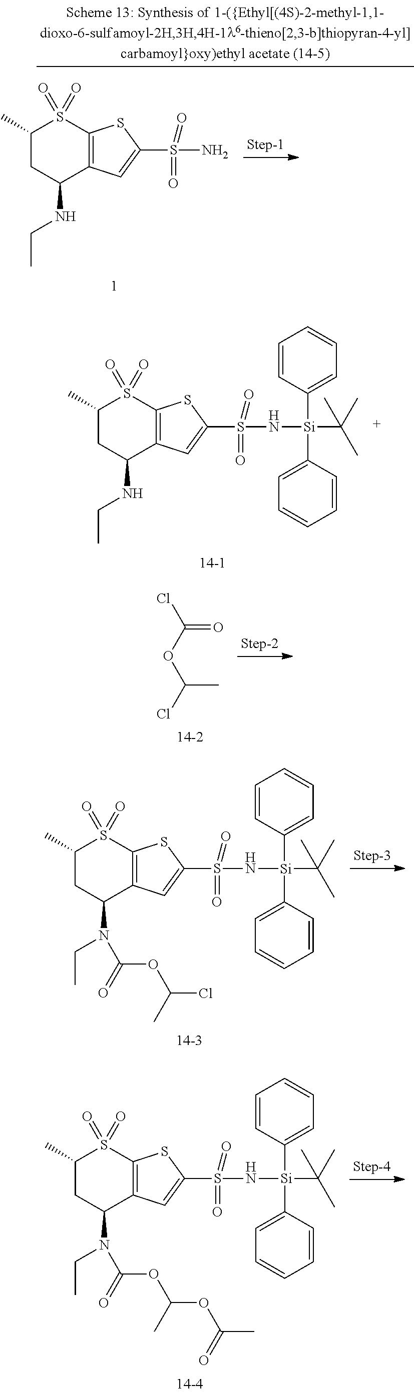

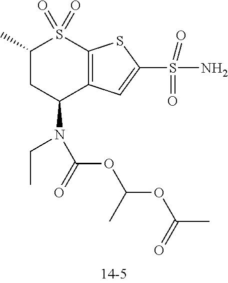 [SMILES: CCN(C(=O)OC(C)OC(C)=O)[C@H]1C[C@H](C)S(=O)(=O)c2sc(S(N)(=O)=O)cc21]

Step 1: Preparation of (2S,4S)-N-(tert-Butyldiphenylsilyl)-4-(ethylamino)-2-methyl-1,1-dioxo-2H,3H,4H-1λ⁶-thieno[2,3-b]thiopyran-6-sulfonamide (14-1): To a solution of dorzolamide 1 (3.0 g, 8.33 mmol) in dichloromethane (10 V) was added N,N-diisopropylethylamine (3.07 mL, 1.67 mmol), tertiary butyldiphenylsilyl chloride (3.29 mL g, 1.25 mmol), and 4-dimethylaminopyridine (0.10 g, 0.83 mmol) were added at 0° C. The reaction mixture was allowed to stir at 25-30° C. over a period of 3 hours. The resulting reaction mass was diluted with ethyl acetate (200 mL), washed with water (100 mL×2), dried over sodium sulfate and concentrated under reduced pressure. The crude product obtained upon evaporation of volatiles was purified through silica gel (230-400 mesh) column chromatography (40% ethyl acetate in hexanes) to obtain product 14-1 as white solid 2.3 g (49%).

Step 2: Preparation of 1-Chloroethyl N-[(4S)-6-[(tert-butyldiphenylsilyl)sulfamoyl]-2-methyl-1,1-dioxo-2H,3H,4H-1λ⁶-thieno[2,3-b]thiopyran-4-yl]-N-ethylcarbamate (14-3): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 14-1 (2.0 g, 3.55 mmol) in dichloromethane (10 V) were added N,N-diisopropylethylamine (1.31 mL, 7.11 mmol) and 1-chloroethyl carbonochloridate 14-2 (0.148 mL, 3.90 mmol) at 0° C. The reaction mixture was allowed to stir at 25-30° C. over a period of 45 minutes. The resulting reaction mass was diluted with ethyl acetate (150 mL), washed with water (80 mL×2), dried over sodium sulfate and concentrated under reduced pressure to obtain product 14-3 as colorless sticky solid 2.0 g. The crude product 14-3 was taken forward to the next step without any further purification.

Step 3: Preparation of 1-({[(2S,4S)-6-[(tert-Butyldiphenylsilyl)sulfamoyl]-2-methyl-1,1-dioxo-2H,3H,4H-1λ⁶-thieno[2,3-b]thiopyran-4-yl](ethyl)carbamoyl}oxy)ethyl acetate (14-4): To a solution of 1-chloroethyl N-[(4S)-6-[(tert-butyldiphenylsilyl)sulfamoyl]-2-methyl-1,1-dioxo-2H,3H,4H-1λ⁶-thieno[2,3-b]thiopyran-4-yl]-N-ethylcarbamate 14-3 (1.8 g, 2.68 mmol) in acetic acid (10 V) was added silver(I)acetate (0.538 g, 3.22 mmol) at 25-30° C. The reaction mixture was allowed to stir at 25-30° C. over a period of 8 hours. The resulting reaction mass was filtered through celite bed. The filtrate was diluted with ethyl acetate (100 mL), washed with water (50 mL×2), dried over sodium sulfate and concentrated under reduced pressure to obtain product 14-4 as an off-white sticky solid 1.4 g. The crude product 14-4 was taken forward to the next step without any further purification.

Step 4: Preparation of 1-({Ethyl[(4S)-2-methyl-1,1-dioxo-6-sulfamoyl-2H,3H,4H-1λ⁶-thieno[2,3-b]thiopyran-4-yl]carbamoyl}oxy)ethyl acetate (14-5): To a solution of 1-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)ethyl acetate 14-4 (1.40 g, 2.02 mmol) in tetrahydrofuran (10 V) was added tetrabutyl ammonium fluoride in 1M THF (2.02 mL, 2.02 mmol) at 0° C. The reaction mixture was allowed to stir at 25-30° C. over a period of 2-3 hours. The resulting reaction mass was diluted with ethyl acetate (200 mL), washed with water (50 mL×2), dried over sodium sulfate and concentrated under reduced pressure. The crude product obtained upon evaporation of volatiles was purified through silica gel (230-400 mesh) column chromatography (40% ethyl acetate in hexanes) to obtain product 14-5 as a white fluffy solid 0.7 g (76%). ¹H NMR (400 MHz, DMSO-d6) δ 8.1-8.0 (m, 2H), 7.31 and 7.26 (2s, 1H), 6.67-6.39 (m, 1H), 5.15-4.72 (m, 1H), 3.96-3.77 (m, 1H), 3.6-3.0 (m, 2H), 2.9-2.7 (m, 1H), 2.5-2.4 (m, 1H), 2.07-1.89 (m, 3H), 1.5-1.0 (m, 9H); m/z [M−H]⁻ 453.1.

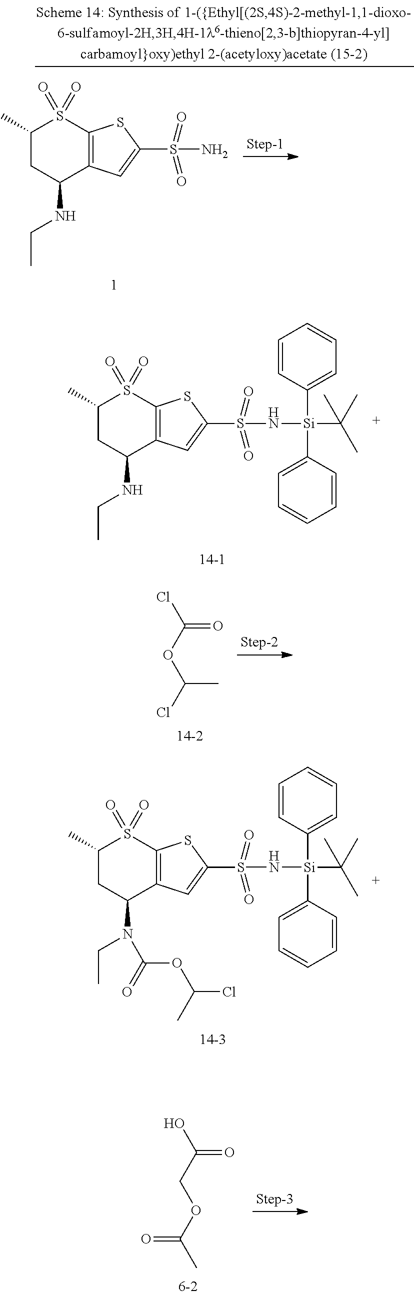

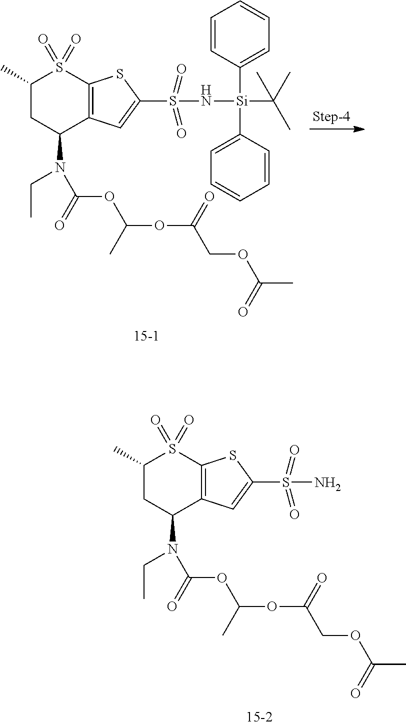

Step 3: Preparation of 1-({[(2S,4S)-6-[(tert-Butyldiphenylsilyl)sulfamoyl]-2-methyl-1,1-dioxo-2H,3H,4H-1λ⁶-thieno[2,3-b]thiopyran-4-yl](ethyl)carbamoyl}oxy)ethyl 2-(acetyloxy)acetate (15-1): To a solution of 1-chloroethyl N-[(4S)-6-[(tert-butyldiphenylsilyl)sulfamoyl]-2-methyl-1,1-dioxo-2H,3H,4H-1λ⁶-thieno[2,3-b]thiopyran-4-yl]-N-ethylcarbamate 14-3 (2.0 g, 2.98 mmol) in acetic acid (25 V) was added silver(I) acetoxy acetate (0.80 g, 3.58 mmol) at 25-30° C. The reaction mixture was allowed to stir at 25-30° C. over a period of 8 hours. The resulting reaction mass was filtered through the celite bed. The filtrate was diluted with ethyl acetate (100 mL), washed with water (50 mL×2), dried over sodium sulfate and concentrated under reduced pressure to obtain product 15-1 as an off-white solid 2.0 g. The crude product 15-1 was taken forward to the next step without any further purification.

Step 4: Preparation of 1-({Ethyl[(2S,4S)-2-methyl-1,1-dioxo-6-sulfamoyl-2H,3H,4H-1λ⁶-thieno[2,3-b]thiopyran-4-yl]carbamoyl}oxy)ethyl 2-(acetyloxy)acetate (15-2): To a solution of 1-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)ethyl 2-(acetyloxy)acetate 15-1 (2.0 g, 2.66 mmol) in tetrahydrofuran (10 V), acetic acid (0.15 mL, 2.66 mmol) and tetrabutyl ammonium fluoride in 1M THF (2.66 mL, 2.66 mmol), were added at 0° C. The reaction mixture was allowed to stir at 25-30° C. over a period of 2-3 hours. The resulting reaction mass was diluted with ethyl acetate (100 mL), washed with water (50 mL×2), dried over sodium sulfate and concentrated under reduced pressure. The crude product obtained upon evaporation of volatiles was purified by silica gel (230-400 mesh) column chromatography (40% ethyl acetate in hexanes) to obtain product 15-2 as a white solid 0.7 g (51%). ¹H NMR (400 MHz, DMSO-d6) δ 8.1-8.0 (m, 2H), 7.36-7.23 (m, 1H), 6.75-6.47 (m, 1H), 5.15-4.8 (m, 1H), 4.8-4.5 (m, 2H), 3.98-3.76 (m, 1H), 3.6-3.0 (m, 2H), 2.9-2.7 (m, 1H), 2.5-2.4 (m, 1H), 2.12-2.04 (m, 3H), 1.5-1.0 (m, 9H); m/z [M+H]⁺ 530.2.

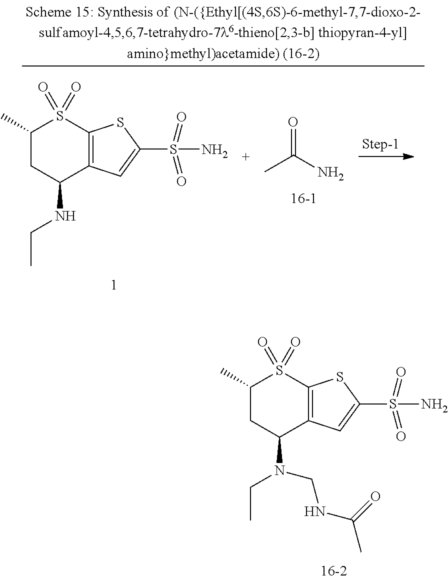

To a solution of acetamide 16-1 (0.32 g, 5.55 mmol) in acetonitrile (30 V) was added aqueous formaldehyde (0.04 mL, 5.55 mmol) at 25-30° C. The resulting reaction mixture was stirred at 80° C. for 3 hours. After 3 hours, dorzolamide 1 (0.2 g, 5.55 mmol) neutralized with N,N-diisopropylethylamine (3.07 mL, 1.67 mmol) and added to reaction mixture at 80° C. and stirred for 16 hours at 80° C. After completion of reaction, reaction mass was concentrated under reduced pressure. The crude product was purified through reverse phase column chromatography to obtain product 16-2 as an off-white solid 63 mg (28%). ¹H NMR (400 MHz, DMSO-d6) δ 8.27 (t, 1H), 8.03 (bs, 2H), 7.53 (s, 1H), 4.27-4.22 (m, 1H), 4.20-4.13 (m, 1H), 4.10-4.03 (m, 1H), 2.6-2.2 (m, 3H), 1.83 (s, 3H), 1.32 (d, 3H), 0.96 (t, 3H); m/z [M+H]⁺ 396.3.

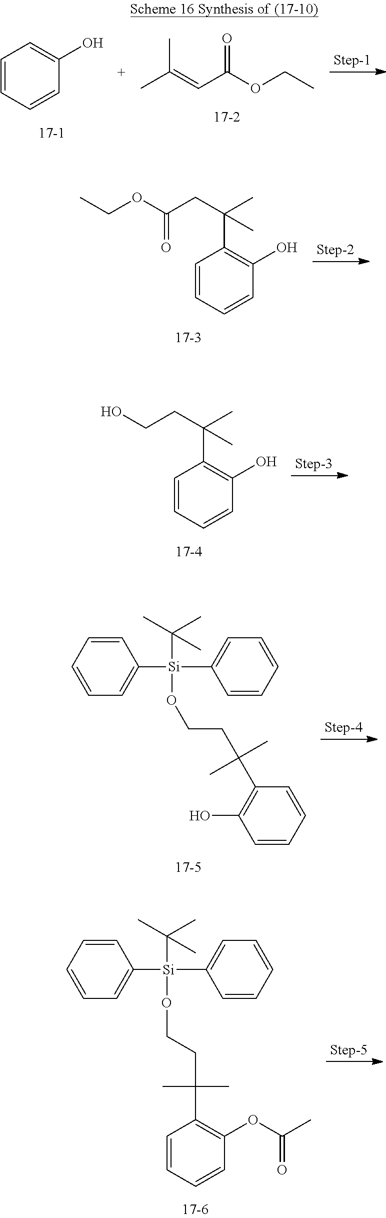

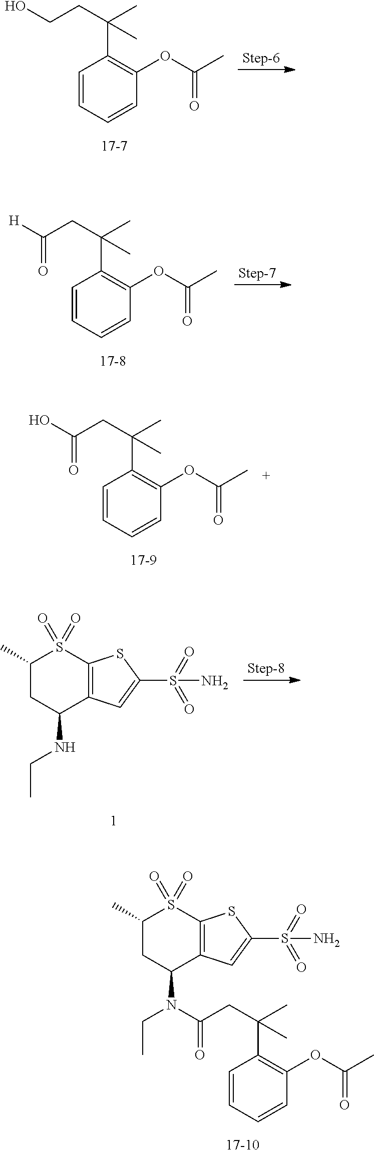 [SMILES: CC(=O)Oc1ccccc1C(C)(C)CCO ; CC(=O)Oc1ccccc1C(C)(C)CC=O ; CC(=O)Oc1ccccc1C(C)(C)CC(=O)O ; CCN[C@H]1C[C@H](C)S(=O)(=O)c2sc(S(N)(=O)=O)cc21 ; CCN(C(=O)CC(C)(C)c1ccccc1OC(C)=O)[C@H]1C[C@H](C)S(=O)(=O)c2sc(S(N)(=O)=O)cc21]

Step 1: Preparation of 4,4-Dimethyl-3,4-dihydro-2H-1-benzopyran-2-one (17-3): To a solution of phenol 17-1 (5.0 g, 4.99 mmol) in methane sulfonic acid (4 V) was added ethyl 3-methylbut-2-enoate 17-2 (6.39 g, 4.9 mmol) at 25-28° C. The reaction mixture was allowed to stir at 70° C. over a period of 2 hours. The resulting reaction mass was quenched with water (100 mL), extracted with ethyl acetate (250 mL×2), dried over sodium sulfate and concentrated under reduced pressure. The crude product obtained upon evaporation of volatiles was purified through silica gel (230-400 mesh) column chromatography (1-3% ethyl acetate/ hexanes) to obtain product 17-3 as a colorless oil, 3.7 g (41.7%).

Step 2: Preparation of 2-(4-Hydroxy-2-methylbutan-2-yl)phenol (17-4): To a solution of lithium aluminium hydride (0.097 g, 0.25 mmol) in dry tetrahydrofuran (5 V) was added 4,4-dimethyl-3,4-dihydro-2H-1-benzopyran-2-one 17-3 (3.7 g, 9.8 mmol) at 0° C. The reaction mixture was allowed to stir at 25-28° C. over a period of 1 hour. The resulting reaction mass was quenched with 1.5 N HCl (20 mL), extracted with ethyl acetate (70 mL×2), dried over sodium sulfate and concentrated under reduced pressure. The crude product 17-4 obtained upon evaporation of volatiles was taken forward to next step 3.03 g (82%)

Step 3: Preparation of 3-[2-(Acetyloxy)phenyl]-3-methylbutanoic acid (17-5): To a solution of 2-(4-hydroxy-2-methylbutan-2-yl)phenol 17-4 (0.30 g, 1.66 mmol) in N,N-dimethyl formamide (5 V), tert-butyldimethylsilyl chloride (0.37 g, 2.49 mmol) and imidazole (0.16 g, 2.4 mmol) were added at 0° C. The reaction mixture was allowed to stir at 25-30° C. over a period of 1 hour. The resulting reaction mass was quenched with water (50 mL), extracted with ethyl acetate (100 mL), dried over sodium sulfate and concentrated under reduced pressure. The crude product 17-5 obtained upon evaporation of volatiles was taken forward to next step 0.39 g (79%).

Step 4: Preparation of 2-{4-[(tert-Butyldimethylsilyl)oxy]-2-methylbutan-2-yl}phenyl acetate (17-6): To a solution of 2-{4-[(tert-butyldimethylsilyl)oxy]-2-methylbutan-2-yl}phenol 17-5 (0.39 g, 1.5 mmol) in dichloromethane (10 V), triethylamine (1.58 mL, 1.56 mmol), 4-dimethylaminopyridine (0.04 g, 0.31 mmol), acetic anhydride (1.19 mL, 1.25 mmol) were added at 0° C. The reaction mixture was then allowed to stir at 25-28° C. over a period of 1 hour. The resulting reaction mass was quenched with water (20 mL), extracted with ethyl acetate (70 mL×2), dried over sodium sulfate and concentrated under reduced pressure. The crude product 17-6 obtained upon evaporation of volatiles was taken forward to next step 0.38 g (86%).

Step 5: Preparation of 2-(4-Hydroxy-2-methylbutan-2-yl)phenyl acetate (17-7): To a solution of 2-{4-[(tert-butyldimethylsilyl)oxy]-2-methylbutan-2-yl}phenyl acetate 17-6 (0.38 g, 4.4 mmol) in tetrahydrofuran (2 V) were added acetic acid (2.28 mL, 6 V) and water (0.76 mL, 2 V) at 0° C. The reaction mixture was allowed to stir at 25-28° C. over a period of 3 hours. The resulting reaction mass was quenched with water (20 mL), extracted with ethyl acetate (70 mL×2), dried over sodium sulfate and concentrated under reduced pressure. The crude product obtained upon evaporation of volatiles was purified by silica gel (230-400 mesh) column chromatography (10% ethyl acetate in hexanes) to obtain product 17-7 as a colorless oil, 0.24 g (95%).

Step 6: Preparation of 2-(4-Hydroxy-2-methylbutan-2-yl)phenyl acetate (17-8): To a solution 2-(2-methyl-4-oxobutan-2-yl)phenyl acetate 17-7 (0.24 g, 1.1 mmol) in dichloromethane (10 V), was added pyridinium chlorochromate (0.54 g, 2.43 mmol) at 0° C. The reaction mixture was allowed to stir at 25-28° C. over a period of 1 hour. The resulting reaction mass was diluted with water (20 mL), extracted with ethyl acetate (70 mL×2), dried over sodium sulfate and concentrated under reduced pressure. The crude product obtained upon evaporation of volatiles was purified through silica gel (230-400 mesh) column chromatography (12% ethyl acetate in hexanes) to obtain product 17-8 as a colorless oil, 0.14 g (58.33%).

Step 7: Preparation of 3-[2-(Acetyloxy)phenyl]-3-methylbutanoic acid (17-9): To a solution of 2-(4-hydroxy-2-methylbutan-2-yl)phenyl acetate 17-8 (0.14 g, 0.63 mmol) in tertiary butanol (20 V), was added 2-methyl butane (0.5 mL, 4.1 V). After 10 minutes sodium chlorite (0.13 g, 1.46 mmol) and sodium dihydrogen phosphate (0.448 mL, 3.2 V, 0.67 M) were added at 25-28° C. The reaction mixture was allowed to stir at 25-28° C. over a period of 1 hour. The resulting reaction mass was quenched with water (20 mL), extracted with ethyl acetate (70 mL×2), dried over sodium sulfate and concentrated under reduced pressure. The crude product obtained upon evaporation of volatiles was purified through silica gel (230-400 mesh) column chromatography (15% ethyl acetate in hexanes) to obtain product 17-9 as an off-white solid, 0.13 g (86.66%).

Step 8: Preparation of 2-(1-{Ethyl[(2s,4s)-2-methyl-1,1-dioxo-6-sulfamoyl-2H,3H,4H-1λ⁶-thieno[2,3-b]thiopyran-4-yl]carbamoyl}-2-methylpropan-2-yl)phenyl acetate (17-10): To a solution of 3-[2-(acetyloxy)phenyl]-3-methylbutanoic acid 17-9 (0.092 g, 0.38 mmol) in dichloromethane (20 mL), were added oxalyl chloride (0.071 mL, 0.83 mmol) and N,N-dimethylformamide (0.001 ml) at 0° C. The reaction mixture was allowed to stir at 25-30° C. over a period of 30 minutes. After completion of reaction, the reaction mixture was concentrated to dryness under nitrogen atmosphere, diluted with dichloromethane (5 V) and added to dorzolamide 1 (0.1 g, 0.27 mmol) neutralized using N,N-diisopropylethylamine (0.099 ml, 0.55 mmol) in dichloromethane (5 V) at 0° C. The reaction mixture was allowed to stir at 25-30° C. over a period of 1 hour. The resulting reaction mass was quenched with water (20 mL), extracted with ethyl acetate (50 mL×2), dried over sodium sulfate and concentrated under reduced pressure. The crude was further purified by reverse phase column chromatography to obtain product 17-10 as an off-white solid, 0.02 g (13%). ¹H NMR (400 MHz, DMSO-d6) δ 8.11 and 8.05 (2s, 2H), 7.4-7.3 (m, 1H), 7.25-7.08 (m, 3H), 7.01-6.94 (m, 1H), 5.4-4.9 (m, 1H), 3.92-3.75 (m, 1H), 3.49-3.33 (m, 1H), 3.24-3.12 (m, 1H), 3.1-2.9 (m, 1H), 2.8-2.7 (m, 1H), 2.68-2.55 (m, 1H), 2.36-2.21 (m, 4H), 1.47-1.25 (m, 9H), 1.15-1.02 (m, 3H); m/z [M+H]⁺ 543.3.

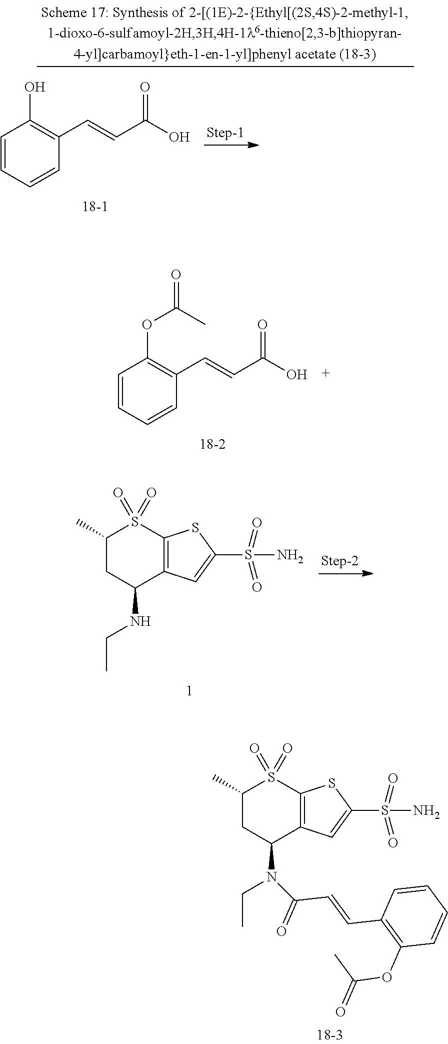 [SMILES: O=C(O)/C=C/c1ccccc1O ; CC(=O)Oc1ccccc1/C=C/C(=O)O ; CCN[C@H]1C[C@H](C)S(=O)(=O)c2sc(S(N)(=O)=O)cc21 ; CCN(C(=O)/C=C/c1ccccc1OC(C)=O)[C@H]1C[C@H](C)S(=O)(=O)c2sc(S(N)(=O)=O)cc21]

Step 1: Preparation of (2E)-3-[2-(Acetyloxy)phenyl]prop-2-enoic acid (18-2): To a solution of (2E)-3-(2-hydroxyphenyl)prop-2-enoic acid 18-1 (3.0 g, 18 mmol) in tetrahydraofuran (10 V) were added triethylamine (5.8 mL, 40 mmol) and acetic anhydride (2.07 mL, 21 mmol) at 0° C. The reaction mixture was allowed to stir at 25-30° C. over a period of 1 hour. The resulting reaction mass was quenched with 1.5N HCl, extracted with ethyl acetate (400 mL). The organic layer was washed with aqueous sodium bicarbonate (200 mL), dried over sodium sulfate and concentrated under reduced to obtain product 18-2 as an off-white solid 1.4 g (38%). ¹H NMR (400 MHz, DMSO-d6) δ 12.54 (bs, 1H), 7.88 (d, 1H), 7.53 (d, 1H), 7.47 (t, 1H), 7.30 (t, 1H), 7.21 (d, 1H), 6.57 (d, 1H), 2.35 (s, 3H); m/z [M+H]⁺ 207.1.

Step 2: Preparation of 2-[(1E)-2-{Ethyl[(2S,4S)-2-methyl-1,1-dioxo-6-sulfamoyl-2H,3H,4H-1λ⁶-thieno[2,3-b]thiopyran-4-yl]carbamoyl}eth-1-en-1-yl]phenyl acetate (18-3): To a solution of (2E)-3-[2-(acetyloxy)phenyl]prop-2-enoic acid 18-2 (1.28 g, 6.0 mmol) in dichloromethane (10 V) were added oxalyl chloride (0.53 mL, 6.2 mmol) and N,N-dimethylformamide (0.07 mL) at 0° C. The reaction mixture was allowed to stir at 25-30° C. over a period of 30 minutes. After completion of reaction, the reaction mixture was concentrated to dryness under nitrogen atmosphere, diluted with dichloromethane (10 V) and added to solution of dorzolamide 1 (1.5 g, 4.1 mmol) neutralized using N,N-diisopropylethylamine(1.0 ml. 6.2 mmol) in dichloromethane (5 V) at 0° C. Reaction mixture was allowed to stir at 25-30° C. over a period of 1 hour. The resulting reaction mass was quenched with water (120 mL), extracted with ethyl acetate (200 mL×2), dried over sodium sulfate and concentrated under reduced pressure. The crude product obtained upon evaporation of volatiles was purified by silica gel (230-400 mesh) column chromatography (60% ethyl acetate in hexane) to obtain product 18-3 as an off-white solid 0.5 g (25%). ¹H NMR (400 MHz, DMSO-d6) δ 8.10-7.97 (m, 3H), 7.61-7.12 (m, 6H), 5.8-5.2 (m, 1H), 4.05-3.88 (m, 1H), 3.75-3.63 (m, 1H), 3.50-3.20 (m, 1H), 3.00-2.65 (m, 1H), 2.65-2.40 (m, 1H), 2.34 (s, 3H), 1.47-1.34 (m, 3H), 1.27-1.02 (m, 3H); m/z [M+H]⁺ 513.3.

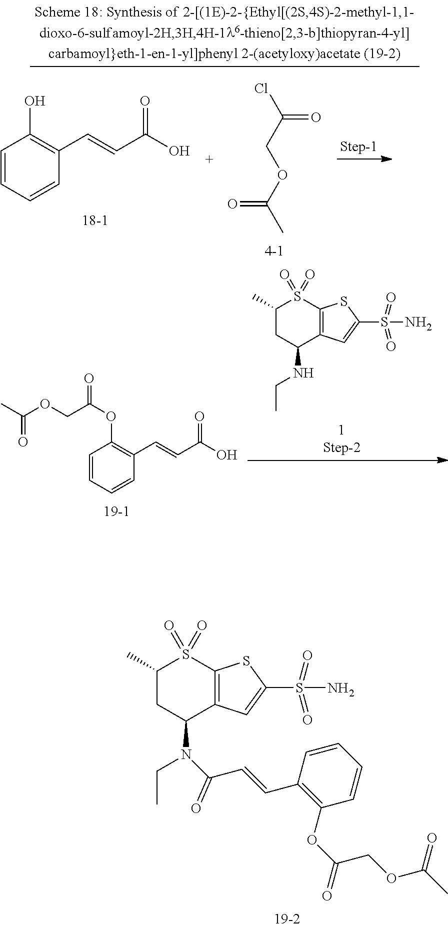

Step 1: Preparation of (2E)-3-(2-{[(acetyloxy)acetyl]oxy}phenyl)prop-2-enoic acid (19-1): To a solution of (2E)-3-(2-hydroxyphenyl)prop-2-enoic acid 18-1 (1.5 g, 9.1 mmol) in tetrahydraofuran (10 V) were added triethyalamine (2.9 mL, 22.0 mmol) and acetoxy acetyl chloride 4-1 (2.1 mL, 20 mmol) at 0° C. The reaction mixture was allowed to stir at 25-30° C. over a period of 1 hour. The resulting reaction mass was concentrated under reduced pressure at 45° C. The crude product obtained upon evaporation of volatiles was purified through reverse phase column chromatography to obtain product 19-1 as a white solid 0.75 g (31%). ¹H NMR (400 MHz, DMSO-d6) δ 12.55 (s, 1H), 7.90 (d, 1H), 7.56 (d, 1H), 7.48 (t, 1H), 7.35 (t, 1H), 7.23 (d, 1H), 6.58 (d, 1H), 5.02 (s, 2H), 2.15 (s, 3H); m/z [M+H]⁺ 265.1.

Step 2: Preparation of 2-[(1E)-2-{Ethyl[(2S,4S)-2-methyl-1,1-dioxo-6-sulfamoyl-2H,3H,4H-1λ⁶-thieno[2,3-b]thiopyran-4-yl]carbamoyl}eth-1-en-1-yl]phenyl 2-(acetyloxy)acetate (19-2): To a solution of (2E)-3-(2-{[(acetyloxy)acetyl]oxy}phenyl)prop-2-enoic acid 19-1 (0.95 g, 3.6 mmol) in dichloromethane (10 V), were added oxalyl chloride (0.71 mL, 8.3 mmol) and N,N-dimethylformamide (0.05 mL) at 0° C. The reaction mixture was allowed to stir at 25-30° C. over a period of 30 minutes. After completion of reaction, reaction mixture was concentrated to dryness under nitrogen atmosphere, diluted with dichloromethane (5 V) and added to the solution of dorzolamide 1 (1.0 g, 2.7 mmol) neutralized using N,N-diisopropylethylamine (1.0 ml. 6.2 mmol) in dichloromethane (5 V) at 0° C. The resulting reaction mixture was allowed to stir at 25-30° C. over a period of 1 hour. The resulting reaction mass was quenched with water (50 mL), extracted with ethyl acetate (100 mL×2), dried over sodium sulfate and concentrated under reduced pressure. The crude product obtained upon evaporation of volatiles was purified through reverse phase column chromatography to afford 19-2 as an off-white solid 0.25 g (14%). ¹H NMR (400 MHz, DMSO-d6) δ 8.11-7.99 (m, 3H), 7.61-7.13 (m, 6H), 5.8-5.2 (m, 1H), 5.01 (s, 2H), 4.06-3.87 (m, 1H), 3.74-3.62 (m, 1H), 3.50-3.20 (m, 1H), 3.00-2.70 (m, 1H), 2.65-2.40 (m, 1H), 2.14 (s, 3H), 1.50-1.36 (m, 3H), 1.27-1.03 (m, 3H); m/z [M+H]⁺ 571.3.

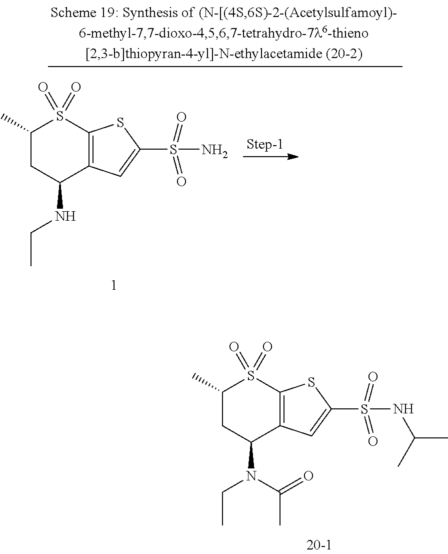 [SMILES: CCN[C@H]1C[C@H](C)S(=O)(=O)c2sc(S(N)(=O)=O)cc21 ; CCN(C(C)=O)[C@H]1C[C@H](C)S(=O)(=O)c2sc(S(=O)(=O)NC(C)C)cc21]

To a solution of dorzolamide 1 (1.0 g, 0.2 mmol) in dichloromethane (10 V) was added triethyl amine (0.39 mL, 2.77 mmol) at 0° C. After 30 minutes, acetic anhydride (0.65 mL, 6.94 mmol) and 4-dimethylaminopyridine (0.03 g, 0.02 mmol) were added at 0° C. The reaction mixture was allowed to stir at 25-30° C. over a period of 1 hour. The resulting reaction was quenched with water (100 mL), extracted with ethyl acetate (300 mL), dried over sodium sulfate and concentrated under reduced pressure. The crude product obtained upon evaporation of volatiles was purified through silica gel (230-400 mesh) column chromatography (2-5% methanol in dichloromethane) to obtain product 20-1 as an off-white solid 0.36 g (31.69%). ¹H NMR (400 MHz, CDCl₃) δ 9.75 (bs, 1H), 7.51 (s, 1H), 5.8-5.7 (m, 1H), 3.74-3.62 (m, 1H), 3.47-3.36 (m, 1H), 3.27-3.14 (m, 1H), 2.90-2.80 (m, 1H), 2.51-2.41 (m, 1H), 2.26 (s, 3H), 2.12 (s, 3H), 1.53 (d, 3H), 1.24 (t, 3H); m/z [M+H]⁺ 409.2.

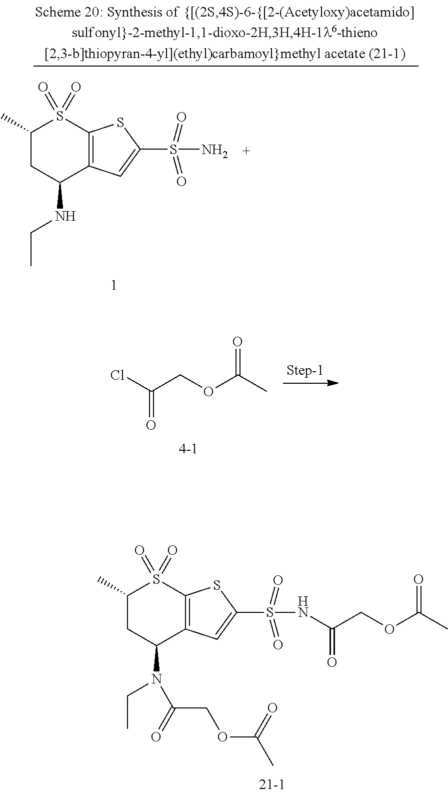

To a solution of dorzolamide 1 (1.5 g, 4.17 mmol) in dichloromethane (10 V) was added N,N-diisopropylethylamine (5.09 mL, 29.19 mmol) at 0° C. After 30 minutes, 2-chloro-2-oxoethyl acetate 4-1 (1.7 mL, 12.51 mmol) and 4-dimethylaminopyridine were added at 0° C. The reaction mixture was allowed to stir at 25-30° C. over a period of 1 hour. After completion of the reaction, the resulting reaction mass was quenched with water (100 mL), extracted with dichloromethane (300 mL), dried over sodium sulfate and concentrated under reduced pressure. The crude product obtained upon evaporation of volatiles was purified through silica gel (230-400 mesh) column (3% methanol in dichloromethane) to obtain product 21-1 as a pale brown solid 1.3 g (59.6%). ¹H NMR (400 MHz, CDCl₃) δ 7.56 (s, 1H), 5.62-5.53 (m, 1H), 4.82 (d, 1H), 4.73 (d, 1H), 4.61 (s, 2H), 3.76-3.64 (m, 1H), 3.46-3.34 (m, 1H), 3.27-3.14 (m, 1H), 2.88-2.77 (m, 1H), 2.53-2.43 (m, 1H), 2.20 (s, 3H), 2.19 (s, 3H), 1.56-1.47 (m, 3H), 1.32-1.21 (m, 3H); m/z [M−H]⁻ 523.2.

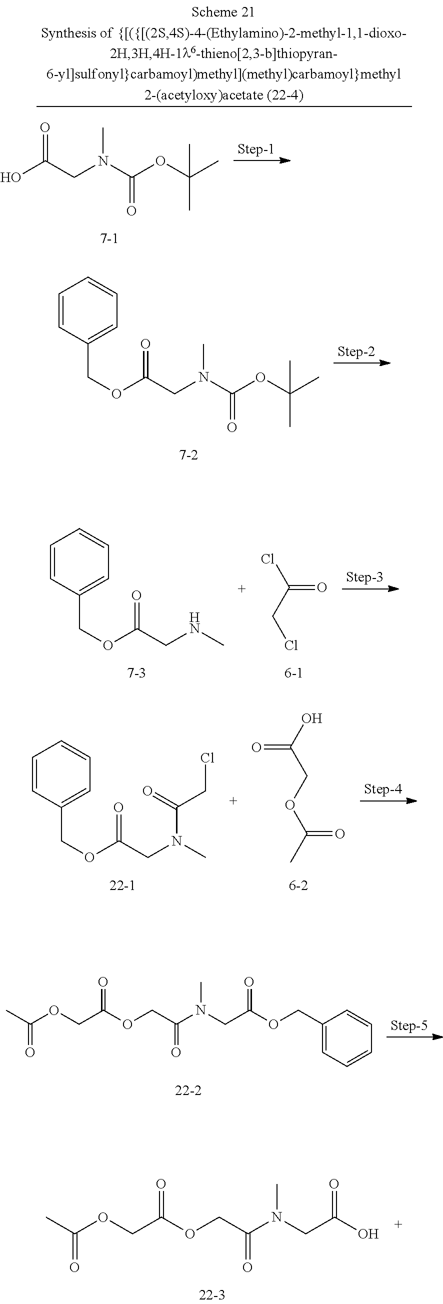

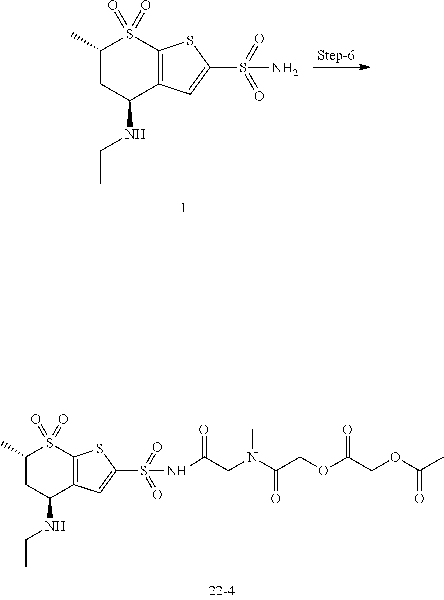 [SMILES: CCN[C@H]1C[C@H](C)S(=O)(=O)c2sc(S(N)(=O)=O)cc21 ; CCN[C@H]1C[C@H](C)S(=O)(=O)c2sc(S(=O)(=O)NC(=O)CN(C)C(=O)COC(=O)COC(C)=O)cc21]

Step 3: Preparation of Benzyl [(chloroacetyl)(methyl)amino]acetate (22-1): To a solution of benzyl (methylamino)acetate 7-3 (10.0 g, 60.54 mmol) in dichloromethane (10 V) were added triethylamine (16.5 mL, 121.08 mmol), N,N-dimethylaminopyridine (0.738 g, 6.05 mmol) and chloroacetyl chloride 6-1 (6.25 mL, 78.7 mmol) at 0° C. The reaction mixture was allowed to stir at 25-30° C. over a period of 1 hour. The resulting reaction mass was quenched with water (300 mL), extracted with ethyl acetate (500 mL×2), dried over sodium sulfate and concentrated under reduced pressure. The crude product obtained upon evaporation of volatiles was purified through silica gel (230-400 mesh) column chromatography (20-30% ethyl acetate in hexane) to obtain product 22-1 as an off-white solid 9.0 g (61.6%).

Step 4: Preparation of 2-{[2-(Benzyloxy)-2-oxoethyl](methyl)amino}-2-oxoethyl (acetyloxy)acetate (22-2): To a solution of benzyl [(chloroacetyl)(methyl)amino]acetate 22-1 (2.5 g, 10.34 mmol) in N,N-dimethylformamide (5 V) were added triethylamine (2.98 mL, 20.68 mmol), sodium iodide (1.54 g, 10.34 mmol) and acetoxyacetic acid 6-2 (1.34 g, 11.37 mmol) at 25-30° C. The reaction mixture was allowed to stir at 55° C. over a period of 2 hours. The resulting reaction mass was diluted with ethyl acetate (200 mL) and washed with water (100 mL×2), dried over sodium sulfate and concentrated under reduced pressure. The crude product obtained upon evaporation of volatiles was purified through silica gel (230-400 mesh) column chromatography (20-25% ethyl acetate in hexane) to obtain product 22-2 as a colorless wax 2.8 g (80.4%).

Step 5: Preparation of [({[(Acetyloxy)acetyl]oxy}acetyl)(methyl)amino]acetic acid (22-3): To a 250 mL Parr shaker vessel were added a solution 2-{[2-(benzyloxy)-2-oxoethyl](methyl)amino}-2-oxoethyl (acetyloxy)acetate 22-2 (2.8 g, 8.30 mmol) in ethyl acetate (10 V) and 10% Pd/C (0.28 g, 50% wet) at 25-30° C. The reaction mixture was stirred at 25-30° C. under hydrogen pressure (5 kg/cm²) over a period of 1 hour. After completion of the reaction, the resulting reaction mixture was filtered through a celite bed and concentrated under reduced pressure to obtain product 22-3 as a waxy solid 1.8 g (90%).

Step 6: Preparation of {[({[(2S,4S)-4-(Ethylamino)-2-methyl-1,1-dioxo-2H,3H,4H-1λ⁶-thieno[2,3-b]thiopyran-6-yl]sulfonyl}carbamoyl)methyl](methyl)carbamoyl}methyl 2-(acetyloxy)acetate (22-4): To a solution of dorzolamide 1 (2.3 g, 6.39 mmol) in dichloromethane (10 V) were added N,N-diisopropylethylamine (1.67 mL, 9.58 mmol), EDC.HCl (1.83 g, 9.58 mmol), [({[(acetyloxy)acetyl]oxy}acetyl)(methyl)amino]acetic acid 22-3 (2.05 g, 8.31 mmol) and 4-dimethylamino pyridine (78 mg, 0.64 mmol) at 0° C. The reaction mixture was allowed to stir at 25-30° C. for 2 hours. After completion of reaction, the resulting reaction mass was concentrated under reduced pressure. The crude product obtained upon evaporation of volatiles was purified by reverse phase column chromatography to obtain product 22-4 as a white solid 1.6 g (45.1%). ¹H NMR (400 MHz, DMSO-d6) δ 8.8 (vbs, 2H), 7.74 and 7.70 (2s, 1H), 4.89 and 4.71 (2s, 4H), 4.64-4.56 (m, 1H), 4.0-3.87 (m, 1H), 3.84-3.70 (m, 2H), 3.26-3.13 (m, 1H), 3.05-2.94 (m, 1H), 2.88 and 2.74 (2s, 3H), 2.61-2.45 (m, 2H), 2.09 (s, 3H), 1.36 (d, 3H), 1.19 (t, 3H); m/z [M+H]⁺ 554.3.

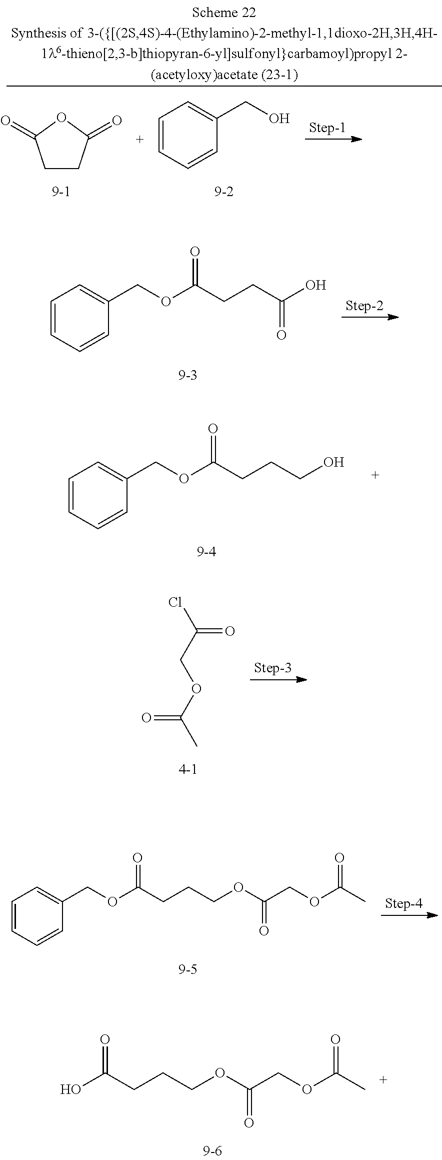

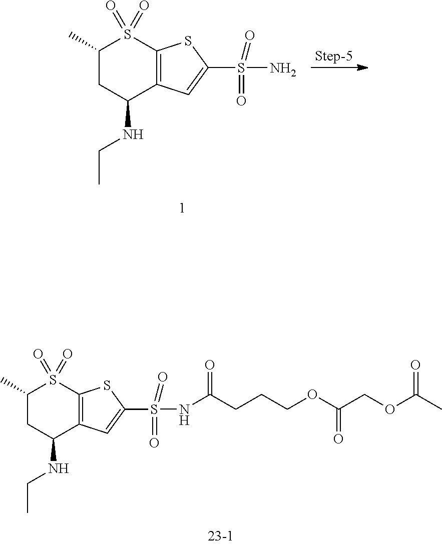 [SMILES: CCN[C@H]1C[C@H](C)S(=O)(=O)c2sc(S(N)(=O)=O)cc21 ; CCN[C@H]1C[C@H](C)S(=O)(=O)c2sc(S(=O)(=O)NC(=O)CCCOC(=O)COC(C)=O)cc21]

Step 5: Preparation of 3-({[(2S,4S)-4-(Ethylamino)-2-methyl-1,1-dioxo-2H,3H,4H-1λ⁶-thieno[2,3-b]thiopyran-6-yl]sulfonyl}carbamoyl)propyl 2-(acetyloxy)acetate (23-1): To a solution of dorzolamide 1 (2.5 g, 6.95 mmol) in dichloromethane (10 V) were added N,N-diisopropylethylamine (2.42 mL, 13.9 mmol), EDC.HCl (1.99 g, 10.42 mmol), 4-dimethylaminopyridine (85 mg, 0.69 mmol) and 4-{[(acetyloxy)acetyl]oxy}butanoic acid 9-6 (1.84 g, 9.03 mmol) at 0° C. The reaction mixture was allowed to stir at 25-30° C. over a period of 1 hour. The resulting reaction mass was diluted with dichloromethane (300 mL), washed with water (100 mL), dried over sodium sulfate and concentrated under reduced pressure. The crude product obtained upon evaporation of volatiles was purified by silica gel (230-400 mesh) column chromatography (3-4% methanol in dichloromethane) to obtain product 23-1 as an off-white solid 1.7 g (48%). ¹H NMR (400 MHz, DMSO-d6) δ 9.2-8.7 (m, 2H), 7.71 (s, 1H), 4.64-4.53 (m, 3H), 4.09-3.93 (m, 3H), 3.25-2.91 (m, 2H), 2.7-2.4 (m, 2H), 2.10-2.02 (m, 5H), 1.78-1.65 (m, 2H), 1.33 (d, 3H), 1.20 (t, 3H); m/z [M+H]⁺ 511.1.

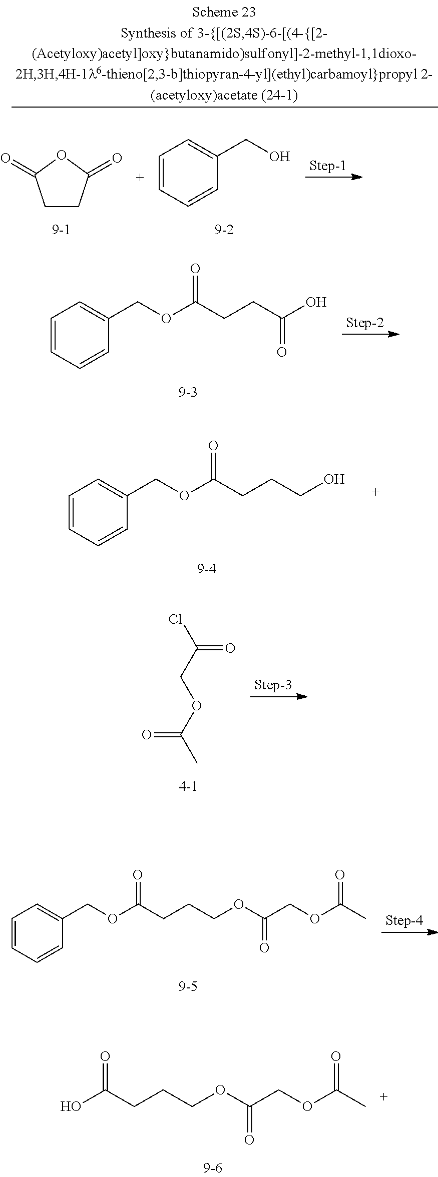

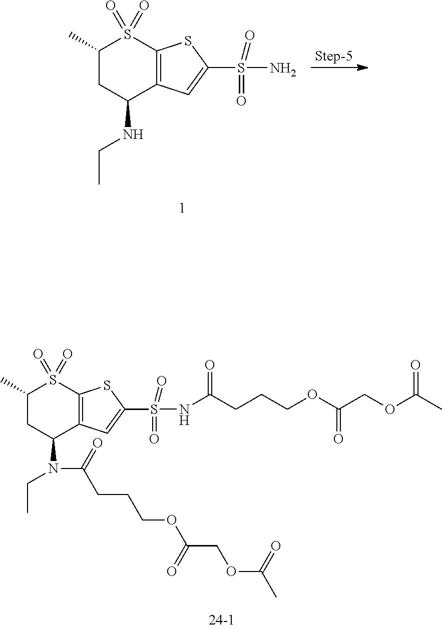 [SMILES: CCN[C@H]1C[C@H](C)S(=O)(=O)c2sc(S(N)(=O)=O)cc21 ; CCN(C(=O)CCCOC(=O)COC(C)=O)[C@H]1C[C@H](C)S(=O)(=O)c2sc(S(=O)(=O)NC(=O)CCCOC(=O)COC(C)=O)cc21]

Step 5: Preparation of 3-{[(2S,4S)-6-[(4-{[2-(Acetyloxy)acetyl]oxy}butanamido)sulfonyl]-2-methyl-1,1-dioxo-2H,3H,4H-1λ⁶-thieno[2,3-b]thiopyran-4-yl](ethyl)carbamoyl}propyl 2-(acetyloxy)acetate (24-1): To a solution of 4-{[(acetyloxy)acetyl]oxy}butanoic acid 9-6 (3.26 g, 15.97 mmol) in dichloromethane (10 V) were added oxalyl chloride (2.47 mL, 19.17 mmol) and N,N-dimethylformamide (0.23 mL) at 0° C. The reaction mixture was allowed to stir at 25-30° C. over a period of 30 minutes. After completion of the reaction, the resulting reaction mass was concentrated under reduced pressure under inert atmosphere. The crude obtained was dissolved in dichloromethane (5V) and added to a solution of dorzolamide 1 (2.3 g, 6.39 mmol), N,N-diisopropylethylamine (6.68 mL, 38.34 mmol) in dichloromethane (5 V) at 0° C. and 4-dimethylaminopyridine (78 mg, 0.63 mmol) at 0° C. The reaction mixture was allowed to stir at 25-30° C. over a period of 1 hour. After completion of the reaction, the resulting reaction mass was quenched with water (100 mL), extracted with dichloromethane (300 mL), dried over sodium sulfate and concentrated under reduced pressure. The crude product obtained upon evaporation of volatiles was purified by reverse phase column chromatography to obtain product 24-1 as an off-white low melting solid 1.2 g (27.2%). ¹H NMR (400 MHz, DMSO-d6) δ 12.7 (bs, 1H), 7.61 and 7.42 (2s, 1H), 5.35-5.02 (m, 1H), 4.64 (s, 2H), 4.60 (s, 2H), 4.19-3.87 (m, 5H), 3.5-3.2 (m, 2H), 2.85-2.70 (m, 1H), 2.55-2.25 (m, 5H), 2.11-2.07 (m, 6H), 1.9-1.7 (m, 4H), 1.39 and 1.37 (2d, 3H), 1.15 (t, 3H); m/z [M+H]⁺ 697.5.

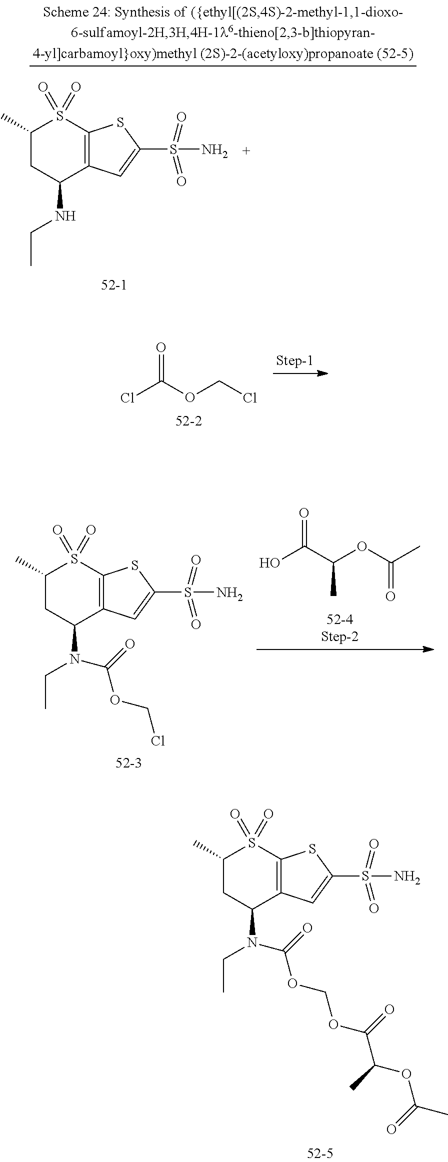

Step-1: Preparation of chloromethyl ethyl[(4S,6S)-6-methyl-7,7-dioxo-2-sulfamoyl-4,5,6,7-tetrahydro-7l6-thieno[2,3-b]thiopyran-4-yl]carbamate (52-3): To a solution of dorzolamide 52-1 (1.4 g, 3.88 mmol) in dichloromethane (25 V) was added N,N-diisopropylethylamine (1.41 mL, 7.7 mmol) at 25-30° C. After 30 min, chloromethyl carbonochloridate (0.38 g, 4.2 mmol) was added at 0° C. The reaction mixture was allowed to stir at 0-5° C. over a period of 1 h. The resulting reaction mass was diluted with ethyl acetate (200 mL) and washed with water (100 mL×2), organic layer was dried over sodium sulfate and concentrated under reduced pressure to obtain compound 52-3 as an off white solid 0.75 g (46%). The crude compound was taken forward to next step without any purification.

Step-2: Preparation of ({ethyl[(2S,4S)-2-methyl-1,1-dioxo-6-sulfamoyl-2H,3H,4H-1λ⁶-thieno[2,3-b]thiopyran-4-yl]carbamoyl}oxy)methyl (2S)-2-(acetyloxy)propanoate (52-5): To a solution of chloromethyl ethyl[(4S,6S)-6-methyl-7,7-dioxo-2-sulfamoyl-4,5,6,7-tetrahydro-7l6-thieno[2,3-b]thiopyran-4-yl]carbamate 52-3 (0.8 g, 1.9 mmol) in N,N-dimethylformamide (3 V) were added sodium iodide (0.43 g, 2.8 mmol), (2S)-2-(acetyloxy)propanoic acid (0.38 mg, 2.8 mmol) and triethylamine (0.54 mL, 3.8 mmol) at 0° C. The reaction mixture was allowed to stir at 55° C. over a period of 3 hours. The resulting reaction mass was diluted with ethyl acetate (100 mL) and washed with water (50 mL×2), organic layer was dried over sodium sulfate and concentrated under reduced pressure. The crude compound was purified by reverse phase column chromatography to obtain product 52-5 as a white solid 0.29 g (29%). ¹H NMR (400 MHz, DMSO-d6/TFA) δ 8.05 (bs, 2H), 7.35 and 7.30 (2s, 1H), 5.81-5.63 (m, 2H), 5.16-4.90 (m, 2H), 3.97-3.77 (m, 1H), 3.4-3.0 (m, 2H), 2.86-2.68 (m, 1H), 2.5-2.4 (m, 1H), 2.07 and 2.05 (2s, 3H), 1.41-1.33 (m, 6H) 1.11 (t, 3H); m/z [M+NH₄]⁺ 530.3.

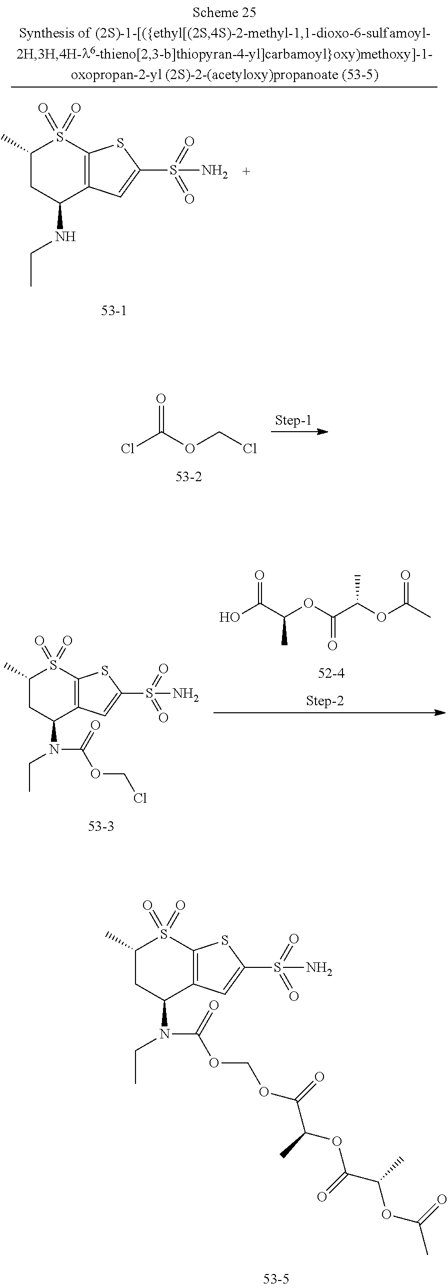 [SMILES: CCN[C@H]1C[C@H](C)S(=O)(=O)c2sc(S(N)(=O)=O)cc21 ; O=C(Cl)OCCl ; CCN(C(=O)OCCl)[C@H]1C[C@H](C)S(=O)(=O)c2sc(S(N)(=O)=O)cc21 ; CC(=O)O[C@@H](C)C(=O)O[C@@H](C)C(=O)O ; CCN(C(=O)OCOC(=O)[C@H](C)OC(=O)[C@H](C)OC(C)=O)[C@H]1C[C@H](C)S(=O)(=O)c2sc(S(N)(=O)=O)cc21]

Step-1: Preparation of chloromethyl ethyl[(4S,6S)-6-methyl-7,7-dioxo-2-sulfamoyl-4,5,6,7-tetrahydro-7l6-thieno[2,3-b]thiopyran-4-yl]carbamate (53-3): To a solution of dorzolamide 53-1 (1.4 g, 3.88 mmol) in dichloromethane (25 V) was added N,N-diisopropylethylamine (1.41 mL, 7.7 mmol) at 25-30° C. After 30 min, chloromethyl carbonochloridate (0.38 g, 4.2 mmol) was added at 0° C. The reaction mixture was allowed to stir at 0-5° C. over a period of 1 h. The resulting reaction mass was diluted with ethyl acetate (200 mL) and washed with water (100 mL×2), organic layer was dried over sodium sulfate and concentrated under reduced pressure to obtain compound 53-3 as an off white solid 0.75 g (46%). The crude compound was taken forward to next step without any purification.

Step-2: Preparation of ({ethyl[(2S,4S)-2-methyl-1,1-dioxo-6-sulfamoyl-2H,3H,4H-1λ⁶-thieno[2,3-b]thiopyran-4-yl]carbamoyl}oxy)methyl (2S)-2-(acetyloxy)propanoate (53-5): To a solution of chloromethyl ethyl[(4S,6S)-6-methyl-7,7-dioxo-2-sulfamoyl-4,5,6,7-tetrahydro-7l6-thieno[2,3-b]thiopyran-4-yl]carbamate 53-3 (0.5 g, 1.2 mmol) in N,N-dimethylformamide (3 V) were added sodium iodide (0.26 g, 1.80 mmol), (2S)-2-(acetyloxy)propanoic acid (0.36. mg, 1.8 mmol) and triethylamine (0.33 mL, 2.4 mmol) at 28 -30° C. The reaction mixture was allowed to stir at 55° C. over a period of 3 hours. The resulting reaction mass was diluted with ethyl acetate (180 mL) and washed with water (50 mL×2), organic layer was dried over sodium sulfate and concentrated under reduced pressure. The crude compound was purified by reverse phase column chromatography to obtain product 53-5 as a white solid 0.12 g (17%). ¹H NMR (400 MHz, DMSO-d6) δ 8.06 (bs, 2H), 7.34 and 7.29 (2s, 1H), 5.83-5.62 (m, 2H), 5.18-4.98 (m, 3H), 3.98-3.80 (m, 1H), 3.4-3.05 (m, 2H), 2.84-2.65 (m, 1H), 2.5-2.4 (m, 1H), 2.07 (s, 3H), 1.48-1.33 (m, 9H) 1.11 (t, 3H); m/z [M+NH₄]⁺ 602.4.

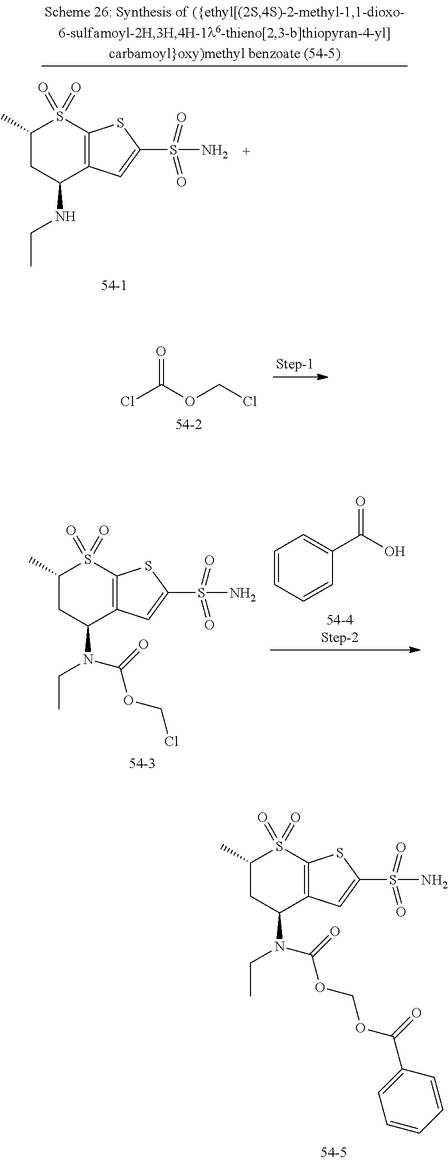

Step-1: Preparation of chloromethyl ethyl[(4S,6S)-6-methyl-7,7-dioxo-2-sulfamoyl-4,5,6,7-tetrahydro-7l6-thieno[2,3-b]thiopyran-4-yl]carbamate (54-3): To a solution of dorzolamide 54-1 (1.4 g, 3.88 mmol) in dichloromethane (25 V) was added N,N-diisopropylethylamine (1.41 mL, 7.7 mmol) at 25-30° C. After 30 min, chloromethyl carbonochloridate (0.38 g, 4.2 mmol) was added at 0° C. The reaction mixture was allowed to stir at 0-5° C. over a period of 1 h. The resulting reaction mass was diluted with ethyl acetate (200 mL) and washed with water (100 mL×2), organic layer was dried over sodium sulfate and concentrated under reduced pressure to obtain compound 54-3 as an off white solid 0.75 g (46%). The crude compound was taken forward to next step without any purification.

Step-2: Preparation ({ethyl[(2S,4S)-2-methyl-1,1-dioxo-6-sulfamoyl-2H,3H,4H-1λ⁶-thieno[2,3-b]thiopyran-4-yl]carbamoyl}oxy)methyl benzoate (54-5): To a solution of chloromethyl ethyl[(4S,6S)-6-methyl-7,7-dioxo-2-sulfamoyl-4,5,6,7-tetrahydro-7l6-thieno[2,3-b]thiopyran-4-yl]carbamate 54-3 (0.5 g, 1.2 mmol) in N,N-dimethylformamide (3 V) were added sodium iodide (0.26 g, 1.80 mmol), benzoic acid (0.21 mg, 1.8 mmol) and triethylamine (0.33 mL, 2.4 mmol) at 28-30° C. The reaction mixture was allowed to stir at 55° C. over a period of 3 hours. The resulting reaction mass was diluted with ethyl acetate (180 mL) and washed with water (50 mL×2), organic layer was dried over sodium sulfate and concentrated under reduced pressure. The crude compound was purified by reverse phase column chromatography to obtain product 54-5 as a white solid 0.13 g (22%). ¹H NMR (400 MHz, DMSO-d6/TFA) δ 8.12-7.87 (m, 4H), 7.72-7.61 (m, 1H), 7.58-7.47 (m, 2H), 7.36 and 7.34 (2s, 1H), 5.99-5.75 (m, 2H), 5.15-5.03 (m, 1H), 3.95-3.78 (m, 1H), 3.40-3.06 (m, 2H), 2.85-2.69 (m, 1H), 2.5-2.4 (m, 1H), 1.41-1.30 (m, 3H) 1.10 (t, 3H); m/z [M+NH₄]⁺ 520.4.

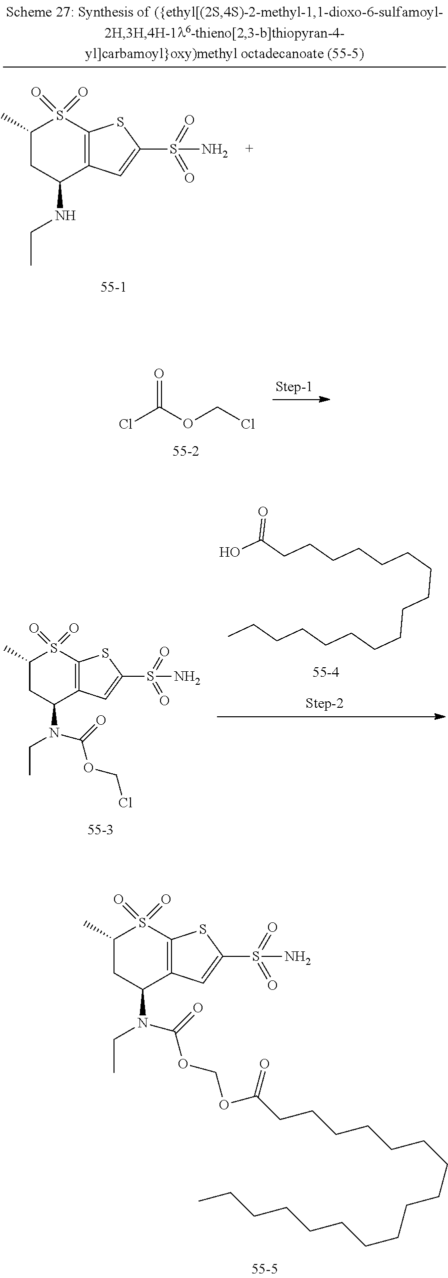

Step-1: Preparation of chloromethyl ethyl[(4S,6S)-6-methyl-7,7-dioxo-2-sulfamoyl-4,5,6,7-tetrahydro-7l6-thieno[2,3-b]thiopyran-4-yl]carbamate (55-3): To a solution of dorzolamide 55-1 (1.4 g, 3.88 mmol) in dichloromethane (25 V) was added N,N-diisopropylethylamine (1.41 mL, 7.7 mmol) at 25-30° C. After 30 min, chloromethyl carbonochloridate (0.38 g, 4.2 mmol) was added at 0° C. The reaction mixture was allowed to stir at 0-5° C. over a period of 1 h. The resulting reaction mass was diluted with ethyl acetate (200 mL) and washed with water (100 mL×2), organic layer was dried over sodium sulfate and concentrated under reduced pressure to obtain compound 55-3 as an off white solid 0.75 g (46%). The crude compound was taken forward to next step without any purification.

Step-2: Preparation ({ethyl[(2S,4S)-2-methyl-1,1-dioxo-6-sulfamoyl-2H,3H,4H-1λ⁶-thieno[2,3-b]thiopyran-4-yl]carbamoyl}oxy)methyl octadecanoate (55-5): To a solution of chloromethyl ethyl[(4S,6S)-6-methyl-7,7-dioxo-2-sulfamoyl-4,5,6,7-tetrahydro-7l6-thieno[2,3-b]thiopyran-4-yl]carbamate 55-3 (0.7 g, 1.68 mmol) in N,N-dimethylformamide (3 V) were added sodium iodide (0.37 g, 2.52 mmol), octadecanoic acid (0.71 mg, 2.52 mmol) and triethylamine (0.47 mL, 3.3 mmol) at 28-30° C. The reaction mixture was allowed to stir at 55° C. over a period of 3 hours. The resulting reaction mass was diluted with ethyl acetate (200 mL) and washed with water (50 mL×2), organic layer was dried over sodium sulfate and concentrated under reduced pressure. The crude compound was purified by reverse phase column chromatography to obtain product 55-5 as a white solid 0.29 g (24%). ¹H NMR (400 MHz, DMSO-d6/TFA) δ 8.06 (bs, 2H), 7.29 (s, 1H), 5.72-5.50 (m, 2H), 5.12-4.96 (m, 1H), 3.96-3.76 (m, 1H), 3.4-3.05 (m, 2H), 2.84-2.69 (m, 1H), 2.5-2.4 (m, 1H), 2.36-2.21 (m, 2H), 1.56-1.40 (m, 2H), 1.40-1.31 (m, 2H), 1.31-1.15 (m, 26H), 1.14-1.02 (m, 3H), 0.88-0.79 (m, 3H); m/z [M+NH₄]⁺ 682.5.

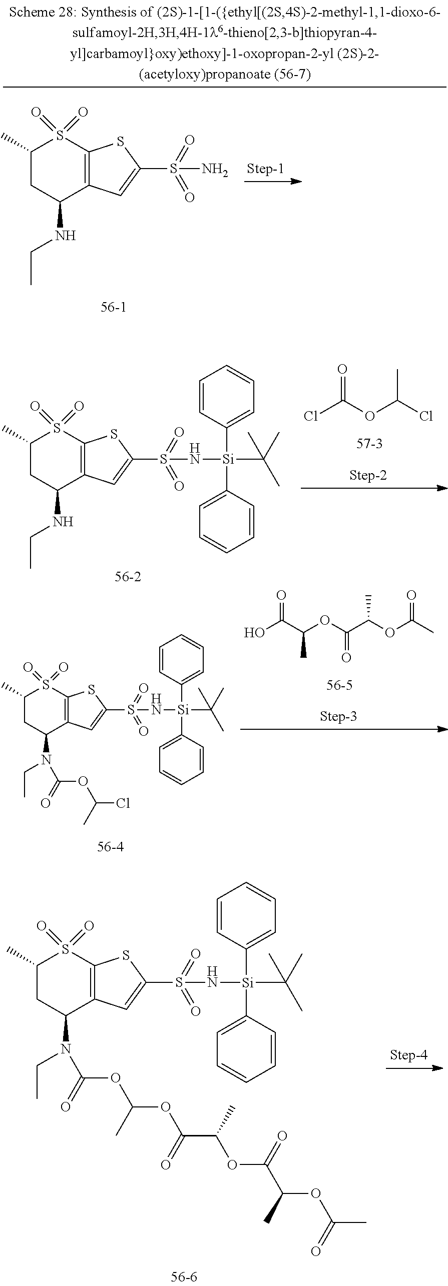

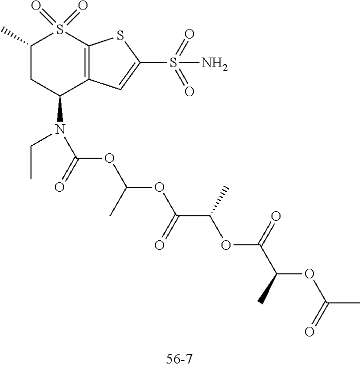 [SMILES: CCN(C(=O)OC(C)OC(=O)[C@H](C)OC(=O)[C@H](C)OC(C)=O)[C@H]1C[C@H](C)S(=O)(=O)c2sc(S(N)(=O)=O)cc21]

Step-1: Preparation of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide (56-2): To a solution of dorzolamide 56-1 (3.0 g, 8.33 mmol) in dichloromethane (10 V) was added N,N-diisopropylethylamine (3.07 mL, 1.67 mmol), tert-Butyl(chloro)diphenylsilane (3.29 mL g, 1.25 mmol), and 4-dimethylaminopyridine (0.10 g, 0.83 mmol) were added at 0° C. The reaction mixture was allowed to stir at 25-30° C. over a period of 3 h. The resulting reaction mass was diluted with ethyl acetate (200 mL), washed with water (100 mL×2), dried over sodium sulfate and concentrated under reduced pressure. The crude product obtained upon evaporation of volatiles was purified through silica gel (230-400 mesh) column chromatography (40% ethyl acetate in hexanes) to obtain product 56-2 as a white solid 2.3 g (49%).

Step-2: Preparation of 1-chloroethyl N-[(4S)-6-[(tert-butyldiphenylsilyl)sulfamoyl]-2-methyl-1,1-dioxo-2H,3H,4H-1λ⁶-thieno[2,3-b]thiopyran-4-yl]-N-ethylcarbamate (56-4): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 56-2 (2.0 g, 3.55 mmol) in dichloromethane (10 V) were added N,N-diisopropylethylamine (1.31 mL, 7.11 mmol), 1-chloroethyl carbonochloridate 56-3 (0.148 mL, 3.90 mmol) at 0° C. The reaction mixture was allowed to stir at 25-30° C. over a period of 45 minutes. The resulting reaction mass was diluted with ethyl acetate (150 mL), washed with water (80 mL×2), dried over sodium sulfate and concentrated under reduced pressure to obtain product 56-4 as a colorless wax 2.0 g. The crude product 56-4 was taken forward to the next step without any further purification.

Step-3: Preparation of (2S)-1-[1-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl} oxy)ethoxy]-1-oxopropan-2-yl (2S)-2-(acetyloxy)propanoate (56-6): To a solution of 1-chloroethyl N-[(4S)-6-[(tert-butyldiphenylsilyl)sulfamoyl]-2-methyl-1,1-dioxo-2H,3H,4H-1λ⁶-thieno[2,3-b]thiopyran-4-yl]-N-ethylcarbamate 56-4 (0.7 g, 1.02 mmol) in tetrahydrofuran (20 V) were added sodium iodide (0.22 g, 1.5 mmol), (2S)-2-{[(2S)-2-(acetyloxy)propanoyl]oxy}propanoic acid 57-5 (0.312 g, 1.5 mmol) followed by triethylamine (0.28 mL, 2.0 mmol) at 25-30° C. The reaction mixture was allowed to stir at 55° C. over a period of 3 h. The resulting reaction mass was diluted with ethyl acetate (250 mL), washed with water (50 mL×2), dried over sodium sulfate and concentrated under reduced pressure to obtain product 56-6 as an off white solid 0.5 g. The crude product 57-6 was taken forward to the next step without any further purification.

Step-4: Preparation of 1-({ethyl[(2S,4S)-2-methyl-1,1-dioxo-6-sulfamoyl-2H,3H,4H-1λ⁶-thieno[2,3-b]thiopyran-4-yl]carbamoyl}oxy)ethyl benzoate (56-7): To a solution of (2S)-1-[1-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)ethoxy]-1-oxopropan-2-yl (2S)-2-(acetyloxy)propanoate 56-6 (0.5 g, 0.59 mmol) in tetrahydrofuran (10 V), were added TBAF (1M THF, 0.59 mL, 0.59 mmol) and acetic acid (0.034 mL, 0.59 mmol) at 0° C. The reaction mixture was allowed to stir at 25-30° C. over a period of 3 h. The resulting reaction mass was diluted with ethyl acetate (200 mL), washed with water (50 mL×2), dried over sodium sulfate and concentrated under reduced pressure. The crude compound was purified by reverse phase column chromatography to obtain product 56-7 as a white solid 0.11 g (29%), as a mixture of stereoisomers. ¹H NMR (400 MHz, DMSO-d6) δ 8.1-8.0 (m, 2H), 7.35-7.22 (m, 1H), 6.71-6.42 (m, 1H), 5.2-4.7 (m, 3H), 3.97-3.75 (m, 1H), 3.5-2.6 (m, 3H), 2.5-2.4 (m, 1H), 2.06 (s, 3H), 1.50-1.32 (m, 9H) 1.32-0.85 (m, 6H); m/z [M−H]⁻ 597.2.

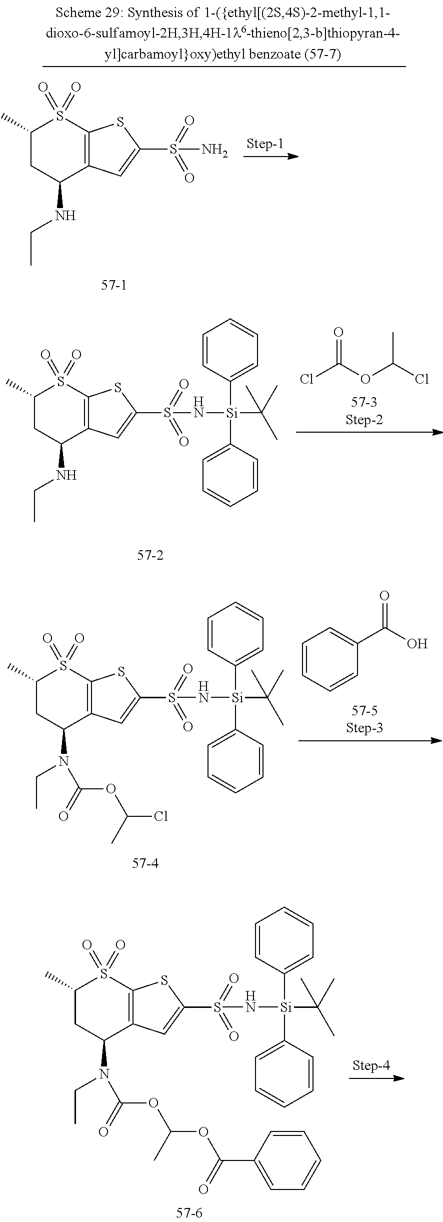

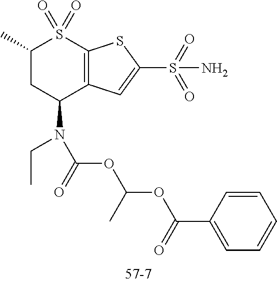 [SMILES: CCN(C(=O)OC(C)OC(=O)c1ccccc1)[C@H]1C[C@H](C)S(=O)(=O)c2sc(S(N)(=O)=O)cc21]

Step-1: Preparation of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide (57-2): To a solution of dorzolamide 57-1 (3.0 g, 8.33 mmol) in dichloromethane (10 V) was added N,N-diisopropylethylamine (3.07 mL, 1.67 mmol), tert-Butyl(chloro)diphenylsilane (3.29 mL g, 1.25 mmol), and 4-dimethylaminopyridine (0.10 g, 0.83 mmol) were added at 0° C. The reaction mixture was allowed to stir at 25-30° C. over a period of 3 h. The resulting reaction mass was diluted with ethyl acetate (200 mL), washed with water (100 mL×2), dried over sodium sulfate and concentrated under reduced pressure. The crude product obtained upon evaporation of volatiles was purified through silica gel (230-400 mesh) column chromatography (40% ethyl acetate in hexanes) to obtain product 57-2 as a white solid 2.3 g (49%).

Step-2: Preparation of 1-chloroethyl N-[(4S)-6-[(tert-butyldiphenylsilyl)sulfamoyl]-2-methyl-1,1-dioxo-2H,3H,4H-1λ⁶-thieno[2,3-b]thiopyran-4-yl]-N-ethylcarbamate (4): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 57-2 (2.0 g, 3.55 mmol) in dichloromethane (10 V) were added N,N-diisopropylethylamine (1.31 mL, 7.11 mmol), 1-chloroethyl carbonochloridate 57-3 (0.148 mL, 3.90 mmol) at 0° C. The reaction mixture was allowed to stir at 25-30° C. over a period of 45 minutes. The resulting reaction mass was diluted with ethyl acetate (150 mL), washed with water (80 mL×2), dried over sodium sulfate and concentrated under reduced pressure to obtain product 57-4 as a colorless wax 2.0 g. The crude product 57-4 was taken forward to the next step without any further purification.

Step-3: Preparation of 1-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)ethyl benzoate (57-6): To a solution of 1-chloroethyl N-[(4S)-6-[(tert-butyldiphenylsilyl)sulfamoyl]-2-methyl-1,1-dioxo-2H,3H,4H-1λ⁶-thieno[2,3-b]thiopyran-4-yl]-N-ethylcarbamate 57-4 (1.0 g, 1.45 mmol) in tetrahydrofuran (20 V) were added Sodium iodide (0.328 g, 2.1 mmol), benzoic acid (0.267 g, 2.1 mmol) followed by triethylamine (0.41 mL, 2.9 mmol) at 25-30° C. The reaction mixture was allowed to stir at 55° C. over a period of 3 h. The resulting reaction mass was diluted with ethyl acetate (200 mL), washed with water (50 mL×2), dried over sodium sulfate and concentrated under reduced pressure to obtain product 57-6 as an off white solid 1.0 g. The crude product 57-4 was taken forward to the next step without any further purification.

Step-4: Preparation of 1-({ethyl[(2S,4S)-2-methyl-1,1-dioxo-6-sulfamoyl-2H,3H,4H-1λ⁶-thieno[2,3-b]thiopyran-4-yl]carbamoyl}oxy)ethyl benzoate (57-7): To a solution of 1-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl]ethyl)carbamoyl}oxy)ethyl benzoate 57-6 (1.0 g, 1.32 mmol) in tetrahydrofuran (10 V), were added TBAF (1M THF, 1.32 mL, 1.32 mmol) and acetic acid (0.07 mL, 1.32 mmol) at 0° C. The reaction mixture was allowed to stir at 25-30° C. over a period of 3 h. The resulting reaction mass was diluted with ethyl acetate (200 mL), washed with water (50 mL×2), dried over sodium sulfate and concentrated under reduced pressure. The crude compound was purified by reverse phase column chromatography to obtain product 57-7 as a white solid 0.14 g (20%), as a mixture of stereoisomers. ¹H NMR (400 MHz, DMSO-d6/TFA) δ 8.12-7.82 (m, 4H), 7.71-7.61 (m, 1H), 7.58-7.43 (m, 2H), 7.41-7.26 (m, 1H), 6.92-6.71 (m, 1H), 5.21-4.75 (m, 1H), 3.95-3.78 (m, 1H), 3.6-3.0 (m, 2H), 2.98-2.77 (m, 1H), 2.5-2.4 (m, 1H), 1.7-1.0 (m, 9H); m/z [M−H]⁻ 515.1.

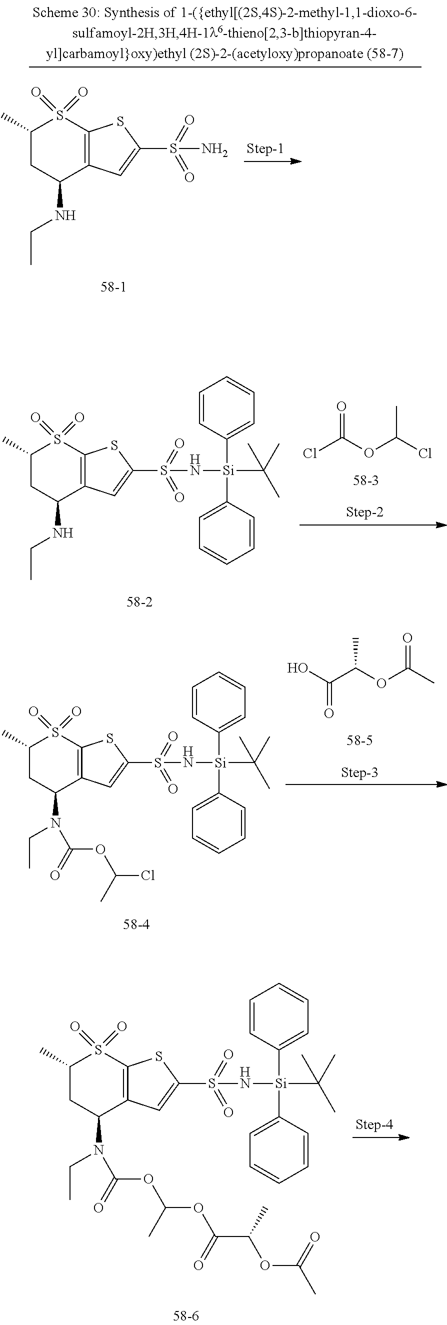

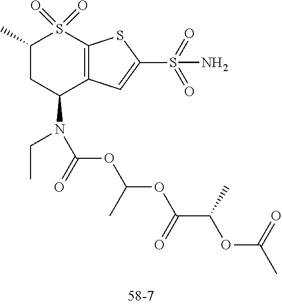 [SMILES: CCN(C(=O)OC(C)OC(=O)[C@H](C)OC(C)=O)[C@H]1C[C@H](C)S(=O)(=O)c2sc(S(N)(=O)=O)cc21]

Step-1: Preparation of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide (58-2): To a solution of dorzolamide 58-1 (3.0 g, 8.33 mmol) in dichloromethane (10 V) was added N,N-diisopropylethylamine (3.07 mL, 1.67 mmol), tert-Butyl(chloro)diphenylsilane (3.29 mL g, 1.25 mmol), and 4-dimethylaminopyridine (0.10 g, 0.83 mmol) were added at 0° C. The reaction mixture was allowed to stir at 25-30° C. over a period of 3 h. The resulting reaction mass was diluted with ethyl acetate (200 mL), washed with water (100 mL×2), dried over sodium sulfate and concentrated under reduced pressure. The crude product obtained upon evaporation of volatiles was purified through silica gel (230-400 mesh) column chromatography (40% ethyl acetate in hexanes) to obtain product 58-2 as a white solid 2.3 g (49%).

Step-2: Preparation of 1-chloroethyl N-[(4S)-6-[(tert-butyldiphenylsilyl)sulfamoyl]-2-methyl-1,1-dioxo-2H,3H,4H-1λ⁶-thieno[2,3-b]thiopyran-4-yl]-N-ethylcarbamate (58-4): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 58-2 (2.0 g, 3.55 mmol) in dichloromethane (10 V) were added N,N-diisopropylethylamine (1.31 mL, 7.11 mmol), 1-chloroethyl carbonochloridate 58-3 (0.148 mL, 3.90 mmol) at 0° C. The reaction mixture was allowed to stir at 25-30° C. over a period of 45 minutes. The resulting reaction mass was diluted with ethyl acetate (150 mL), washed with water (80 mL×2), dried over sodium sulfate and concentrated under reduced pressure to obtain product 58-4 as a colorless wax 2.0 g. The crude product 58-4 was taken forward to the next step without any further purification.

Step-3: Preparation of 1-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)ethyl (2S)-2-(acetyloxy)propanoate (58-6): To a solution of 1-chloroethyl N-[(4S)-6-[(tert-butyldiphenylsilyl)sulfamoyl]-2-methyl-1,1-dioxo-2H,3H,4H-1λ⁶-thieno[2,3-b]thiopyran-4-yl]-N-ethylcarbamate 58-4 (0.5 g, 0.74 mmol) in tetrahydrofuran (20 V) were added sodium iodide (0.16 g, 1.1 mmol), (2S)-2-(acetyloxy)propanoic acid 58-5 (0.14 g, 1.1 mmol) followed by triethylamine (0.21 mL, 1.49 mmol) at 25-30° C. The reaction mixture was allowed to stir at 55° C. over a period of 3 h. The resulting reaction mass was diluted with ethyl acetate (250 mL), washed with water (50 mL×2), dried over sodium sulfate and concentrated under reduced pressure to obtain product 58-6 as an off white solid 0.50 g. The crude product 58-6 was taken forward to the next step without any further purification.

Step-4: Preparation of 1-({ethyl[(2S,4S)-2-methyl-1,1-dioxo-6-sulfamoyl-2H,3H,4H-1λ⁶-thieno[2,3-b]thiopyran-4-yl]carbamoyl}oxy)ethyl (2S)-2-(acetyloxy)propanoate (58-7): To a solution of 1-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)ethyl (2S)-2-(acetyloxy)propanoate 58-6 (0.5 g, 0.65 mmol) in tetrahydrofuran (10 V), were added TBAF (1M THF, 0.65 mL, 0.65 mmol) and acetic acid (0.037 mL, 0.65 mmol) at 0° C. The reaction mixture was allowed to stir at 25-30° C. over a period of 3 h. The resulting reaction mass was diluted with ethyl acetate (200 mL), washed with water (50 mL×2), dried over sodium sulfate and concentrated under reduced pressure. The crude compound was purified by reverse phase column chromatography to obtain product 58-7 as a white solid 0.21 g (61%), as a mixture of stereoisomers. ¹H NMR (400 MHz, DMSO-d6) δ 8.1-8.0 (m, 2H), 7.35-7.22 (m, 1H), 6.71-6.47 (m, 1H), 5.20-4.76 (m, 2H), 3.96-3.75 (m, 1H), 3.55-2.6 (m, 3H), 2.5-2.4 (m, 1H), 2.09-1.96 (m, 3H), 1.5-0.9 (m, 12H); m/z [M+NH₄]⁺ 544.3.

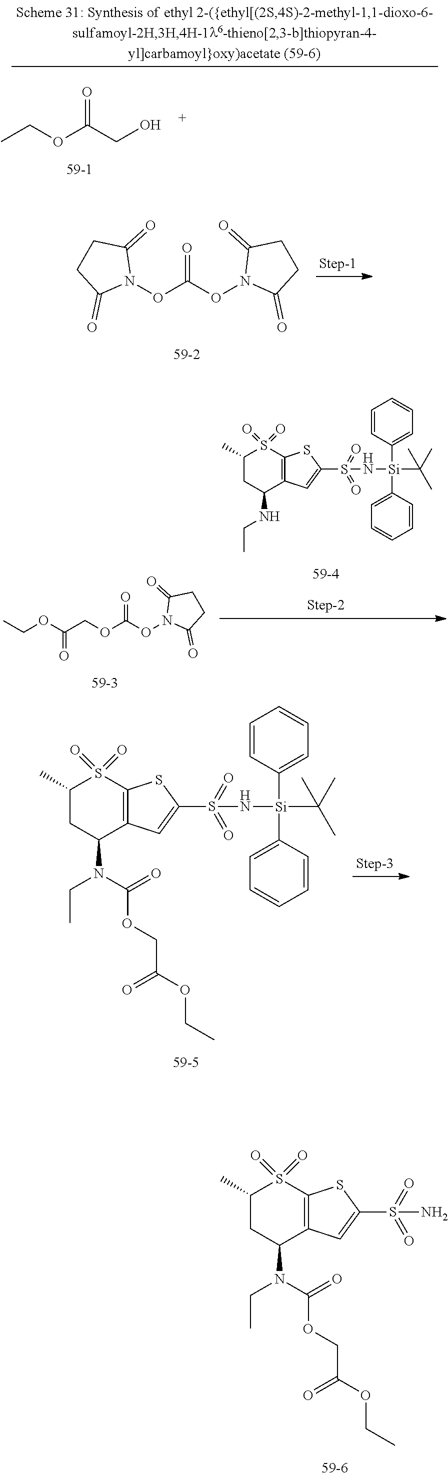

Step-1: Preparation of ethyl 2-({[(2,5-dioxopyrrolidin-1-yl)oxy]carbonyl}oxy)acetate (59-3): To a solution of ethyl 2-hydroxyacetate 59-1 (0.4 g, 3.84 mmol) in THF (10 V) were added pyridine (0.62 mL, 7.69 mmol) and bis(2,5-dioxopyrrolidin-1-yl) carbonate 59-2 (1.97 g, 0.62 mmol) at 25-30° C. The resulting reaction mixture was allowed to stir at 25-30° C. over a period of 16 h. The reaction mass was quenched with 1% H3PO4 solution (50 mL), extracted with ethyl acetate (100 mL×2), dried over sodium sulfate and concentrated under reduced pressure. The crude product was purified by silica gel column chromatography (60-120 mesh) to obtain product 59-3 as an off-white solid 0.6 g (63%).

Step-2: Preparation ethyl 2-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)acetate (59-5): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 59-4 (0.5 g, 0.88 mmol) in THF (10V) were added pyridine (0.072 mL, 0.88 mmol), DMAP (0.01 g, 0.088 mmol) and ethyl 2-({[(2,5-dioxopyrrolidin-1-yl)oxy]carbonyl}oxy)acetate 59-3 (0.327 g, 1.33 mmol) at 25-30° C. The reaction mixture was allowed to stir at 25-30° C. over a period of 16 h. The reaction was quenched with water (50 mL), extracted with ethyl acetate (200 mL), dried over sodium sulfate and concentrated under reduced pressure to obtain crude product 59-5 as an off white wax 0.60 g. The crude product 59-5 was taken forward to the next step without any further purification.

Step-3: Preparation ethyl 2-({ethyl[(2S,4S)-2-methyl-1,1-dioxo-6-sulfamoyl-2H,3H,4H-1λ⁶-thieno[2,3-b]thiopyran-4-yl]carbamoyl}oxy)acetate (59-6): To a solution of ethyl 2-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)acetate 59-5 (0.6 g, 0.86 mmol) in tetrahydrofuran (10 mL) were added acetic acid (0.02 mL, 0.43 mmol) and TBAF (0.42 mL, 0.43 mmol) at 0-5° C. The reaction mixture was stirred at 0-5° C. for 30 min. The resulting reaction mass was diluted with ethyl acetate (100 mL), washed with water (2×50 mL), dried over sodium sulphate and concentrated under reduced pressure. The crude product obtained upon evaporation of volatiles was by reverse phase column chromatography to obtain product 59-6 as a pinkish puffy solid 0.16 g (39.70%). ¹H NMR (400 MHz, DMSO-d6/TFA) δ 8.05 (bs, 2H), 7.5-7.3 (m, 1H), 5.25-5.02 (m, 1H), 4.75-4.42 (m, 2H), 4.17-4.04 (m, 2H), 3.96-3.82 (m, 1H), 3.45-3.0 (m, 2H), 2.92-2.72 (m, 1H), 2.5-2.4 (m, 1H), 1.40-1.33 (m, 3H) 1.23-1.00 (m, 6H); m/z [M+H]⁺ 455.1.

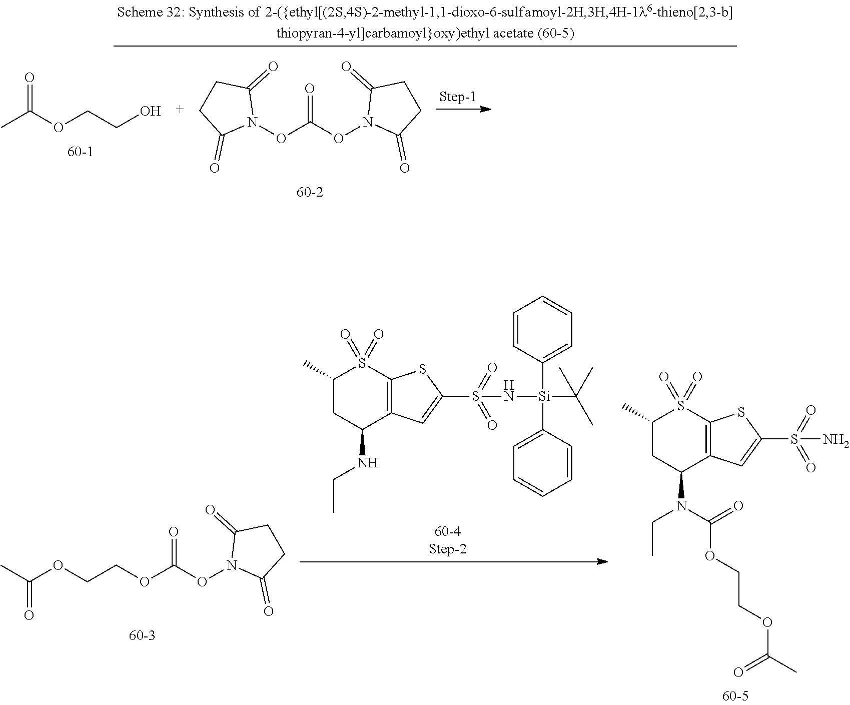

Step-1: Preparation of 2-({[(2,5-dioxopyrrolidin-1-yl)oxy]carbonyl}oxy)ethyl acetate (60-3): To a solution of 2-hydroxyethyl acetate 60-1 (0.5 g, 4.80 mmol) in THF (10V) were added pyridine (0.78 mL, 9.61 mmol) and bis(2,5-dioxopyrrolidin-1-yl) carbonate 60-2 (2.46 g, 9.61 mmol) at 25-30° C. The reaction mixture was allowed to stir at 25-30° C. over a period of 16 h. The resulting reaction mass was quenched with 1% H₃PO₄ solution (30 mL), extracted with ethyl acetate (150 mL×2), dried over sodium sulfate and concentrated under reduced pressure. The crude product was purified by silica gel column chromatography (230-400 mesh) to obtain product 60-3 as a colorless liquid 0.6 g (51%).

Step-2: Preparation 2-({ethyl[(2S,4S)-2-methyl-1,1-dioxo-6-sulfamoyl-2H,3H,4H-1λ⁶-thieno[2,3-b]thiopyran-4-yl]carbamoyl}oxy)ethyl acetate (60-5): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 60-4 (0.5 g, 0.088 mmol) in THF (10V) were added pyridine (0.07 mL, 0.88 mmol), DMAP (0.01 g, 0.088 mmol) and 2-({[(2,5-dioxopyrrolidin-1-yl)oxy]carbonyl}oxy)ethyl acetate 60-3 (.326 g, 1.33 mmol) at 25-30° C. The reaction mixture was allowed to stir at 25-30° C. over a period of 16 h. After completion of the reaction, the reaction was quenched with water (50 mL), extracted with ethyl acetate (250 mL), dried over sodium sulfate and concentrated under reduced pressure. The crude was purified by reverse phase column chromatography to obtain product 60-5 as a white solid 200 mg (49%). ¹H NMR (400 MHz, DMSO-d6/TFA) δ 8.05 (bs, 2H), 7.31 (s, 1H), 5.14-4.78 (m, 1H), 4.31-3.76 (m, 5H), 3.5-3.0 (m, 2H), 2.89-2.70 (m, 1H), 2.5-2.4 (m, 1H), 2.04-1.89 (m, 3H), 1.37 (d, 3H), 1.16-1.04 (m, 3H); m/z [M+NH₄]⁺ 472.1.

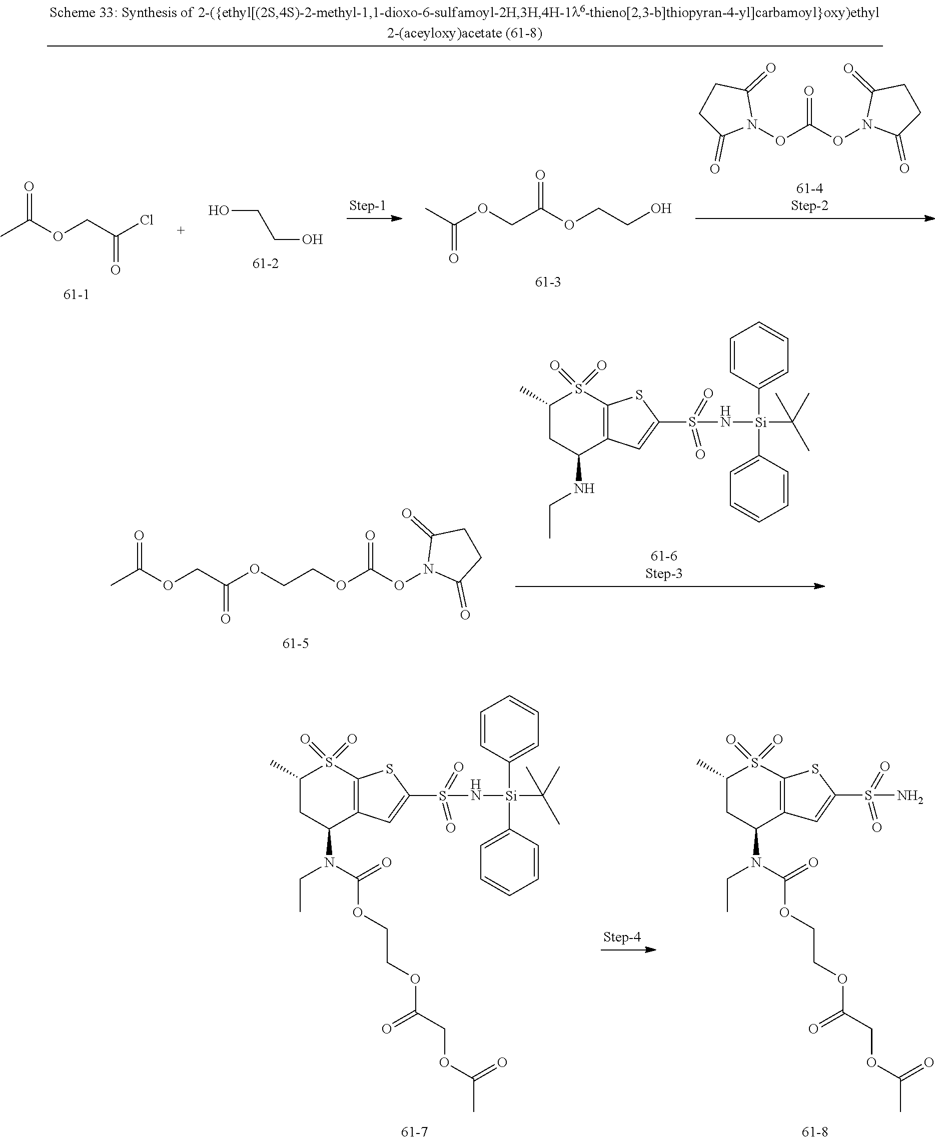

Step-1: Preparation of 2-hydroxyethyl 2-(acetyloxy)acetate (61-3): To a solution of ethane-1,2-diol 61-2 (0.822 mL, 14.70 mmol) in dichloromethane (10 V) were added TEA (2.12 mL, 14.70 mmol) and 2-chloro-2-oxoethyl acetate 61-1 (1.0 g, 7.35 mmol) at 0° C. The reaction mixture was allowed to stir at 25-30° C. over a period of 16 h. After completion of the reaction, reaction mass was quenched with water (100 mL), extracted with dichloromethane (300 mL), dried over sodium sulfate and concentrated under reduced pressure. The crude product was purified by silica gel column chromatography (230-400 mesh) to obtain product 61-3 as a colorless liquid 0.8 g (67.22%).

Step-2: Preparation of 2-({[(2,5-dioxopyrrolidin-1-yl)oxy]carbonyl}oxy)ethyl 2-(acetyloxy)acetate (61-5): To a solution of 2-hydroxyethyl 2-(acetyloxy)acetate 61-3 (0.8 g, 4.93 mmol) in THF (10V) were added pyridine (0.8 mL, 9.86 mmol) and bis(2,5-dioxopyrrolidin-1-yl) carbonate 61-4 (2.52 g, 9.86 mmol) was added at 25-30° C. The reaction mixture was allowed to stir at 25-30° C. over a period of 16 h. The resulting reaction mass was quenched with 1% H₃PO₄ solution (50 mL), extracted with ethyl acetate (200 mL×2), dried over sodium sulfate and concentrated under reduced pressure. The crude product was purified by silica gel column chromatography (230-400) to obtain product 61-5 as a colorless liquid 0.6 g (47.24%).

Step-3: Preparation of 2-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)ethyl 2-acetyloxy)acetate (61-7): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 61-6 (0.5 g, 0.88 mmol) in THF (10V) were added pyridine (0.072 mL, 0.88 mmol), DMAP (0.01 g, 0.088 mmol) and 2-({[(2,5-dioxopyrrolidin-1-yl)oxy]carbonyl}oxy)ethyl 2-(acetyloxy)acetate 61-5 (0.404 g, 1.33 mmol) at 25-30° C. The reaction mixture was allowed to stir at 25-30° C. over a period of 16 h. After completion of the reaction, the reaction was quenched with water (50 mL), extracted with ethyl acetate (300 mL), dried over sodium sulfate and concentrated under reduced pressure to obtain crude product 61-7 as an off white wax 0.6 g. The crude product 7 was taken forward to the next step without any further purification.

Step-4: Preparation 2-({ethyl[(2S,4S)-2-methyl-1,1-dioxo-6-sulfamoyl-2H,3H,4H-1λ⁶-thieno[2,3-b]thiopyran-4-yl]carbamoyl}oxy)ethyl 2-(acetyloxy)acetate (61-8): To a solution of 2-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl]ethyl)carbamoyl}oxy)ethyl 2-acetyloxy)acetate 61-7 (0.6 g, 0.8 mmol) in tetrahydrofuran (10 mL) were added acetic acid (0.02 mL, 0.4 mmol) and TBAF (0.38 mL, 0.4 mmol) at 0-5° C. The reaction mixture was stirred at 0-5° C. for 30 min. The resulting reaction mass was diluted with ethyl acetate (100 mL), washed with water (2×50 mL), dried over sodium sulphate and concentrated under reduced pressure. The crude product obtained upon evaporation of volatiles was by reverse phase column chromatography to obtain product 61-8 as a white solid 0.18 g (39.47%). ¹H NMR (400 MHz, DMSO-d6) δ 8.06 (bs, 2H), 7.31 (s, 1H), 5.10-4.85, (m, 1H), 4.68-4.55 (m, 2H), 4.40-3.78 (m, 5H), 3.5-3.05 (m, 2H), 2.87-2.70 (m, 1H), 2.5-2.4 (m, 1H), 2.10-2.02 (m, 3H), 1.38 (d, 3H), 1.12 (t, 3H); m/z [M+NH₄]⁺ 530.1.

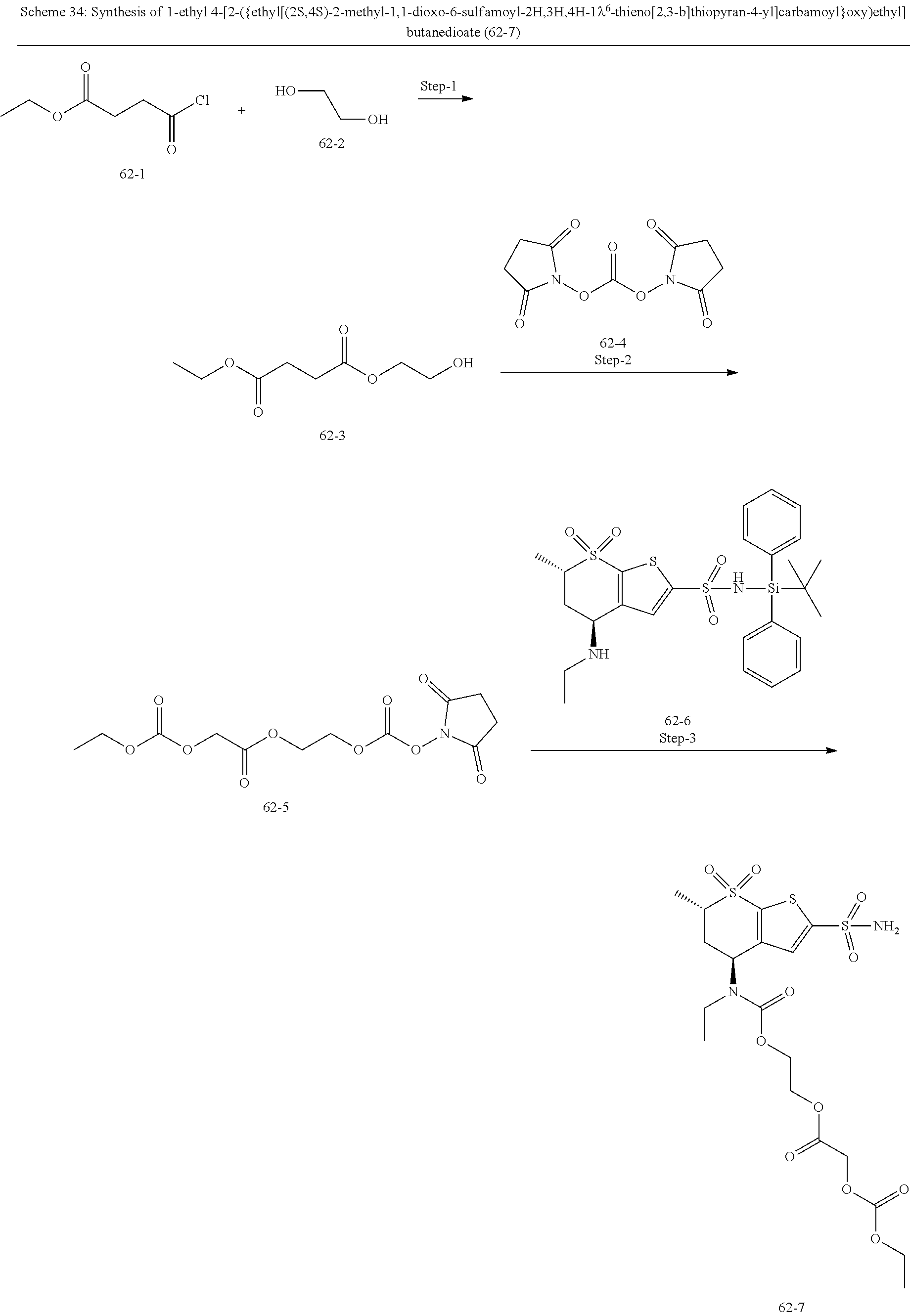

Step-1: Preparation of 1-ethyl 4-(2-hydroxyethyl) butanedioate (62-3): To a solution of ethane-1,2-diol 62-2 (0.684 mL, 12.15 mmol) in dichloromethane (10 V) were added triethylamine (1.75 mL, 12.15 mmol) and ethyl 4-chloro-4-oxobutanoate 62-1 (1.0 g, 6.07 mmol) at 0° C. The reaction mixture was allowed to stir at 25-30° C. over a period of 16 h. The resulting reaction mass was quenched with water (100 mL), extracted with dichloromethane (300 mL), dried over sodium sulfate and concentrated under reduced pressure. The crude product was purified by silica gel column chromatography (230-400 mesh) to obtain product 62-3 as a colorless liquid 0.95 g (82.60%).

Step-2: Preparation of 1-[2-({[(2,5-dioxopyrrolidin-1-yl)oxy]carbonyl}oxy)ethyl] 4-ethyl butanedioate (62-5): To a solution of 1-ethyl 4-(2-hydroxyethyl) butanedioate 62-3 (0.950 g, 4.99 mmol) in THF (10V) were added pyridine (0.813 mL, 9.99 mmol) and bis(2,5-dioxopyrrolidin-1-yl) carbonate 62-4 (2.55 g, 9.99 mmol) was added at 25-30° C. The reaction mixture was allowed to stir at 25-30° C. over a period of 16 h. The resulting reaction mass was quenched with 1% H3PO4 solution (50 mL), extracted with ethyl acetate (200 mL×2), dried over sodium sulfate and concentrated under reduced pressure. The crude product was purified by silica gel column chromatography (230-400 mesh) to obtain product 62-5 as a colorless liquid 0.7 g (57.57%).

Step-3: Preparation of 1-ethyl 4-[2-({ethyl[(2S,4S)-2-methyl-1,1-dioxo-6-sulfamoyl-2H,3H,4H-1λ⁶-thieno[2,3-b]thiopyran-4-yl]carbamoyl}oxy)ethyl] butanedioate (62-7): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 62-6 (0.4 g, 0.711 mmol) in THF (10V) were added pyridine (0.058 mL, 0.711 mmol), DMAP (0.0086 g, 0.071 mmol) and 1-[2-({[(2,5-dioxopyrrolidin-1-yl)oxy]carbonyl}oxy)ethyl] 4-ethyl butanedioate 62-5 (0.404 g, 1.06 mmol) at 25-30° C. The reaction mixture was allowed to stir at 25-30° C. over a period of 16 h. After completion of the reaction, the reaction was quenched with water (50 mL), extracted with ethyl acetate (100 mL), dried over sodium sulfate and concentrated under reduced pressure. The crude compound was purified by reverse phase column chromatography to obtain product 62-7 as an off white solid 0.12 g (31.25%). ¹H NMR (400 MHz, DMSO-d6) δ 8.05 (bs, 2H), 7.31 (s, 1H), 5.12-4.90, (m, 1H), 4.32-3.78 (m, 7H), 3.5-3.05 (m, 2H), 2.87-2.71 (m, 1H), 2.6-2.4 (m, 5H), 1.38 (d, 3H), 1.20-1.04 (m, 6H); m/z [M+H]⁺ 541.2.

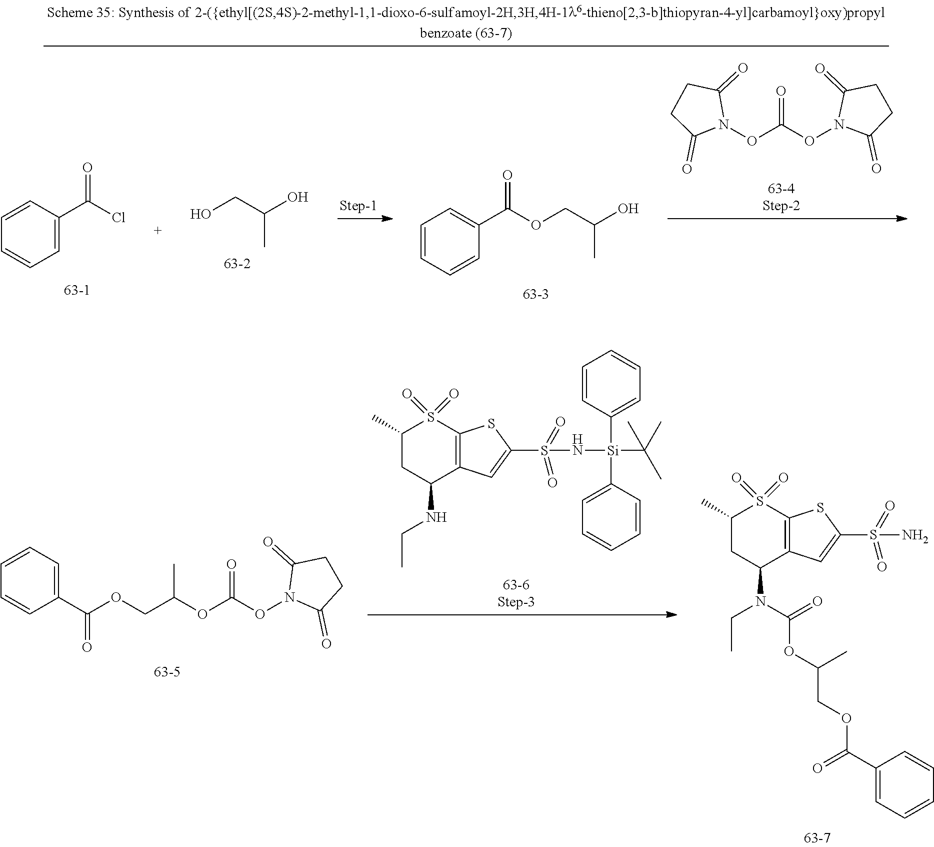

Step-1: Preparation of 2-hydroxypropyl benzoate (63-3): To a solution of propane-1,2-diol 63-2 (0.97 mL, 7.11 mmol) in dichloromethane (10 V) were added triethylamine (1.9 mL, 14.23 mmol) and benzoyl chloride 63-1 (0.9 mL, 14.23 mmol) at 0° C. The reaction mixture was allowed to stir at 25-30° C. over a period of 4 h. After completion of the reaction, the resulting reaction mass was quenched with water (100 mL) and extracted with ethyl acetate (200 mL), dried over sodium sulfate and concentrated under reduced pressure. The crude product obtained upon evaporation of volatiles was purified by silica gel (230-400 mesh) column chromatography to obtain product 63-3 as a colorless liquid 1.1 g (85.9%).

Step-2: Preparation 2-({[(2,5-dioxopyrrolidin-1-yl)oxy]carbonyl}oxy)propyl benzoate (63-5): To a solution of 2-hydroxypropyl benzoate 63-3 (1.1 g, 4.65 mmol) in tetrahydrofuran (10 V) were added pyridine (1.85 mL, 18.31 mmol) and bis(2,5-dioxopyrrolidin-1-yl) carbonate 63-4 (3.9 g, 15.2 mmol) at 0° C. The reaction mixture was allowed to stir at 25-30° C. over a period of 16 h. After completion of the reaction, the resulting reaction mass was quenched with water (100 mL), extracted with ethyl acetate (200 mL), dried over sodium sulfate and concentrated under reduced pressure. The crude product obtained upon evaporation of volatiles was purified by silica gel (230-400 mesh) column chromatography to obtain product 63-5 as a colorless wax 1.5 g (76.5%).

Step-3: Preparation of 2-({ethyl[(2S,4S)-2-methyl-1,1-dioxo-6-sulfamoyl-2H,3H,4H-1λ⁶-thieno[2,3-b]thiopyran-4-yl]carbamoyl}oxy)propyl benzoate (63-7): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 63-6 (0.5 g, 0.88 mmol) in tetrahydrofuran (10 V) were added pyridine (0.18 mL, 1.77 mmol), 2-({[(2,5-dioxopyrrolidin-1-yl)oxy]carbonyl}oxy)propyl benzoate 63-5 (0.42 g, 1.33 mmol) and 4-dimethylaminopyridine (26 mg, 0.213 mmol) at 0° C. The reaction mixture was allowed to stir at 25-30° C. over a period of 48 h. After completion of the reaction, the resulting reaction mass was quenched with water (100 mL) and extracted with ethyl acetate (200 mL), dried over sodium sulfate and concentrated under reduced pressure. The crude was purified by preparative HPLC to obtain product 63-7 as an off white solid 170 mg (36%) as a mixture of stereo- and regio-isomers. ¹H NMR (400 MHz, DMSO-d6) δ 8.2-7.88 (m, 4H), 7.70-7.61 (m, 1H), 7.58-7.44 (m, 2H), 7.33-7.23 (m, 1H), 5.4-3.7 (m, 5H), 3.5-3.05 (m, 2H), 3.03-2.65 (m, 1H), 2.45-2.28 (m, 1H), 1.4-0.7 (m, 9H); m/z [M+NH₄]⁺ 548.2.

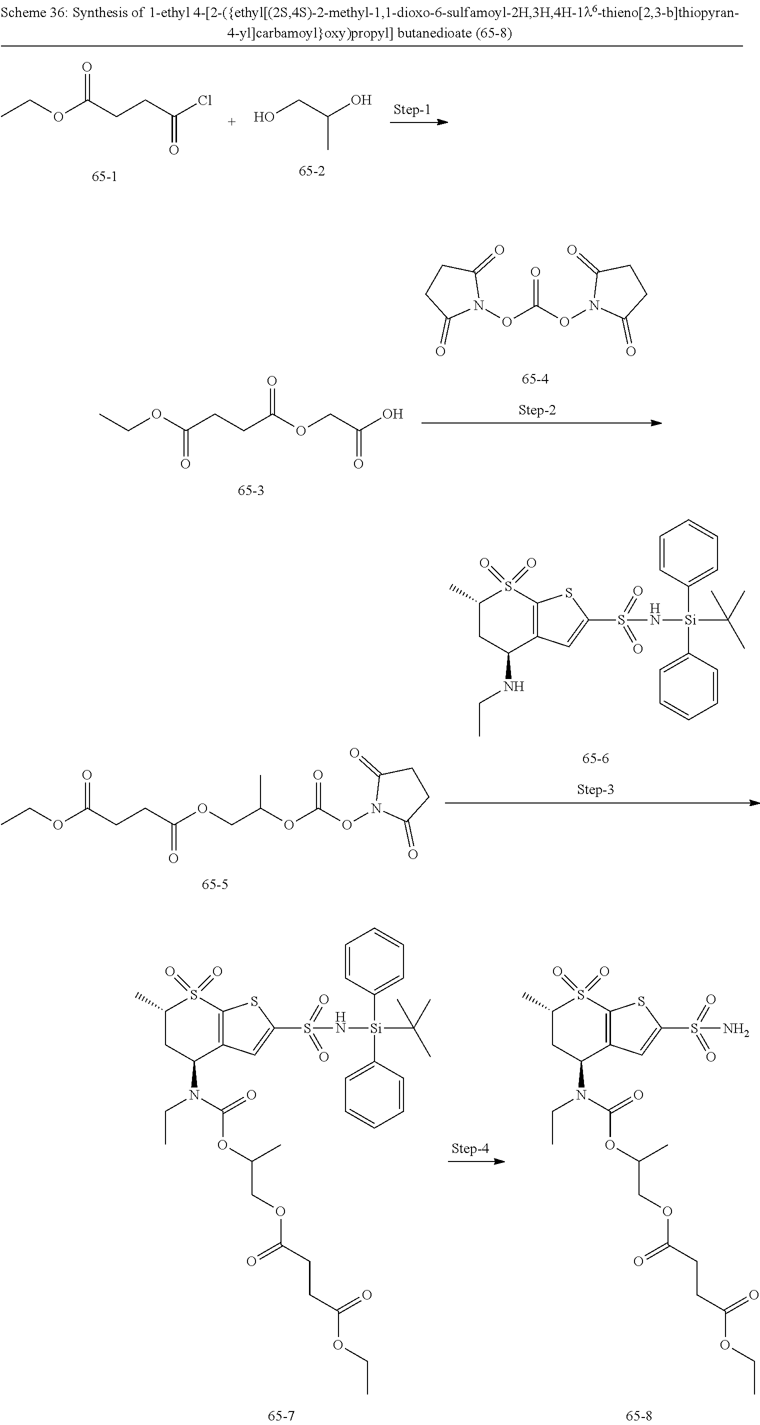

Step-1: Preparation of 1-ethyl 4-(2-hydroxypropyl) butanedioate (65-3): To a solution of propane-1,2-diol 65-2 (1.7 mL, 24.30 mmol) in dichloromethane (10 V) were added TEA (3.5 mL, 24.30 mmol) and ethyl 4-chloro-4-oxobutanoate 65-1 (1.7 mL, 12.15 mmol) at 0° C. The reaction mixture was allowed to stir at 25-30° C. over a period of 2 h. The resulting reaction mass was quenched with water (100 mL), extracted with dichloromethane (300 mL), dried over sodium sulfate and concentrated under reduced pressure. The crude product was purified by silica gel column chromatography (230-400 mesh) to obtain product 65-3 as a colorless liquid 2.0 g (80.0%).

Step-2: Preparation of 1-[2-({[(2,5-dioxopyrrolidin-1-yl)oxy]carbonyl}oxy)propyl] 4-ethyl butanedioate (65-5): To a solution of 1-ethyl 4-(2-hydroxypropyl) butanedioate 65-3 (2.0 g, 9.80 mmol) in THF (10V) were added pyridine (1.59 mL, 19.60 mmol), DMAP (0.23 g, 1.96 mmol) and bis(2,5-dioxopyrrolidin-1-yl) carbonate 65-4 (5.1 g, 19.60 mmol) at 25-30° C. The reaction mixture was allowed to stir at 25-30° C. over a period of 16 h. The resulting reaction mass was quenched with 1% H3PO4 solution (50 mL), extracted with ethyl acetate (200 mL×2), dried over sodium sulfate and concentrated under reduced pressure. The crude product was purified by silica gel column chromatography (230-400 mesh) to obtain product 65-5 as a colorless liquid 2.5 g (73%).

Step-3: Preparation of 1-[2-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)propyl] 4-ethyl butanedioate (65-7): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 65-6 (0.6 g, 1.06 mmol) in THF (10V) were added pyridine (0.21 mL, 2.13 mmol), DMAP (0.026 g, 0.213 mmol) and 1-[2-({[(2,5-dioxopyrrolidin-1-yl)oxy]carbonyl}oxy)propyl] 4-ethyl butanedioate 65-5 (0.552 g, 1.6 mmol) at 25-30° C. The reaction mixture was allowed to stir at 25-30° C. over a period of 48 h. After completion of the reaction, the reaction was quenched with water (50 mL), extracted with ethyl acetate (300 mL), dried over sodium sulfate and concentrated under reduced pressure to obtain crude compound 65-7 (800 mg). The crude compound was carried as such into next step without any further purification.

Step-4: Preparation of 1-ethyl 4-[2-({ethyl[(2S,4S)-2-methyl-1,1-dioxo-6-sulfamoyl-2H,3H,4H-1λ⁶-thieno[2,3-b]thiopyran-4-yl]carbamoyl}oxy)propyl] butanedioate (65-8): To a solution of 1-[2-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)propyl] 4-ethyl butanedioate 65-7 (0.8 g, 1.0 mmol) in THF (10V) were added acetic acid (0.029 mL, 0.5 mmol), TBAF (0.5 mL, 0.5 mmol) at 0° C. The reaction mixture was allowed to stir at 25-30° C. over a period of 30 min. The reaction mass was quenched with water (50 mL), extracted with ethyl acetate (250 mL), dried over sodium sulfate and concentrated under reduced pressure. The crude compound was purified by reverse phase column chromatography to obtain product 65-8 as a mixture of regio- and stereo-isomers as an off white solid 270 mg (48%). ¹H NMR (400 MHz, DMSO-d6) δ 8.04 (bs, 2H), 7.33-7.24 (m, 1H), 5.17-4.65, (m, 2H), 4.3-3.7 (m, 5H), 3.6-2.7 (m, 3H), 2.6-2.4 (m, 5H), 1.41-1.33 (m, 3H), 1.25-0.9 (m, 9H); m/z [M+H]⁺ 555.2.

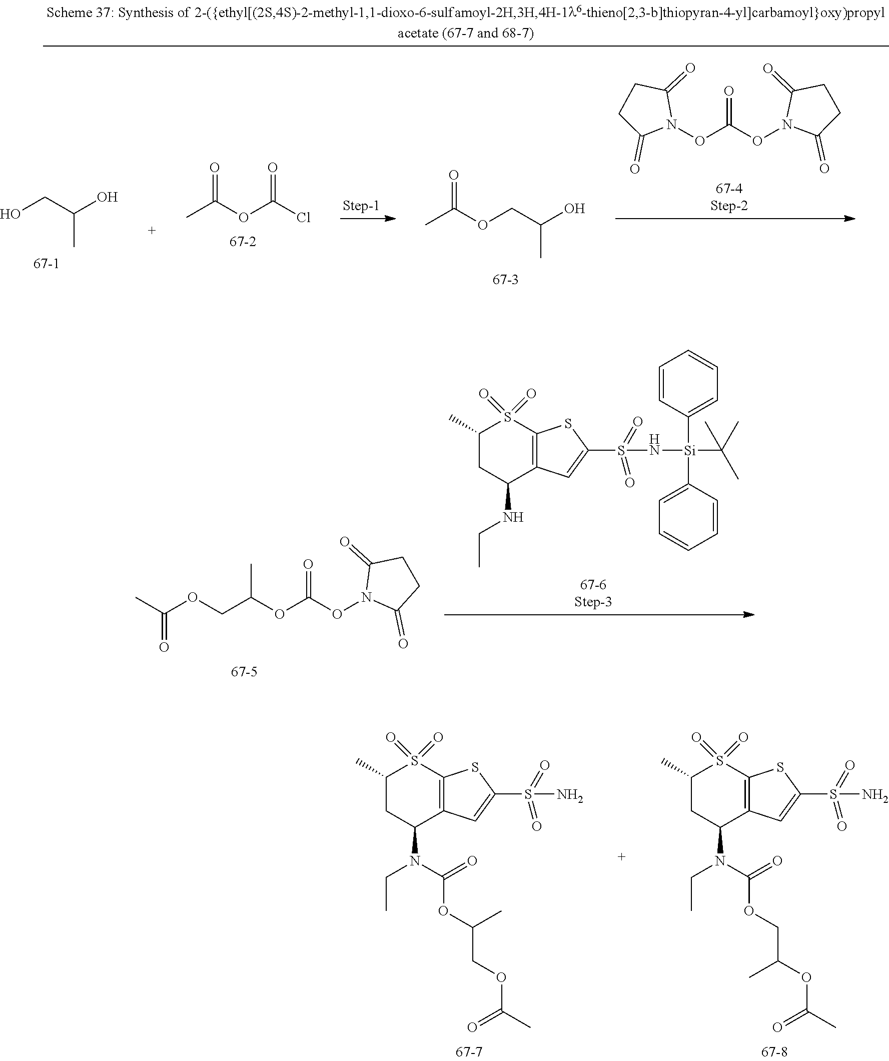

Step-1: Preparation of 2-hydroxypropyl acetate (67-3): To a solution of propane-1,2-diol 67-1 (1 mL, 13.14 mmol) in acetonitrile (10 V) were added DIPEA (0.484 mL, 2.62 mmol) and acetic anhydride 67-2 (0.621 mL, 6.57 mmol) at 0° C. The reaction mixture was allowed to stir at 40° C. over a period of 16 h. The resulting reaction mass was quenched with water (100 mL), extracted with dichloromethane (300 mL), dried over sodium sulfate and concentrated under reduced pressure. The crude product was purified by silica gel column chromatography (230-400 mesh) to obtain product 65-3 as a colorless liquid 0.8 g (51.61%).

Step-2: Preparation of 2-({[(2,5-dioxopyrrolidin-1-yl)oxy]carbonyl}oxy)propyl acetate (67-5): To a solution of 2-hydroxypropyl acetate 67-3 (0.800 g, 6.77 mmol) in THF (10V) were added pyridine (1.1 mL, 13.55 mmol) and bis(2,5-dioxopyrrolidin-1-yl) carbonate 67-4 (5.20 g, 20.33 mmol) at 25-30° C. The reaction mixture was allowed to stir at 25-30° C. over a period of 16 h. The resulting reaction mass was quenched with 1% H3PO4 solution (50 mL), extracted with ethyl acetate (200 mL×2), dried over sodium sulfate and concentrated under reduced pressure. The crude product was purified by silica gel column chromatography (230-400 mesh) to obtain product 67-5 as a colorless liquid 0.6 g (34.28%).

Step-3: Preparation of 2-({ethyl[(2S,4S)-2-methyl-1,1-dioxo-6-sulfamoyl-2H,3H,4H-1λ⁶-thieno[2,3-b]thiopyran-4-yl]carbamoyl}oxy)propyl acetate (67-7 and 68-7): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 67-6 (0.5 g, 0.889 mmol) in THF (10V) were added pyridine (0.0725 mL, 0.88 mmol), DMAP (0.021 g, 0.177 mmol) and 2-({[(2,5-dioxopyrrolidin-1-yl)oxy]carbonyl}oxy)propyl acetate 67-5 (0.404 g, 1.06 mmol) at 25-30° C. The reaction mixture was allowed to stir at 25-30° C. over a period of 16 h. After completion of the reaction, the reaction was quenched with water (50 mL), extracted with ethyl acetate (150 mL), dried over sodium sulfate and concentrated under reduced pressure. Isolated compound 67-7 and 67-7 in two fractions (Isomer-1 and Isomer-2) by preparative HPLC in 28 mg and 50 mg scale respectively (16.31%). Fractions are composed of differing mixtures of regio- and stereo-isomers of the propylene glycol group. Fraction 1. ¹H NMR (400 MHz, DMSO-d6) δ 8.07 (bs, 2H), 7.26 (s, 1H), 5.16-4.66, (m, 2H), 4.2-2.7 (m, 6H), 2.5-2.4 (m, 1H), 1.99 (s, 3H), 1.38 (d, 3H), 1.3-0.6 (m, 6H); m/z [M+NH₄]⁺ 486.1. Fraction 2. ¹H NMR (400 MHz, DMSO-d6) δ 8.01 (bs, 2H), 7.29 and 7.26 (2s, 1H), 5.14-4.71, (m, 2H), 4.3-3.0 (m, 5H), 2.95-2.71 (m, 1H), 2.5-2.4 (m, 1H), 2.03 and 1.96 (2s, 3H), 1.38 (d, 3H), 1.25-1.02 (m, 6H); m/z [M+H]⁺ 469.1 and [M+NH₄]⁺ 486.1.

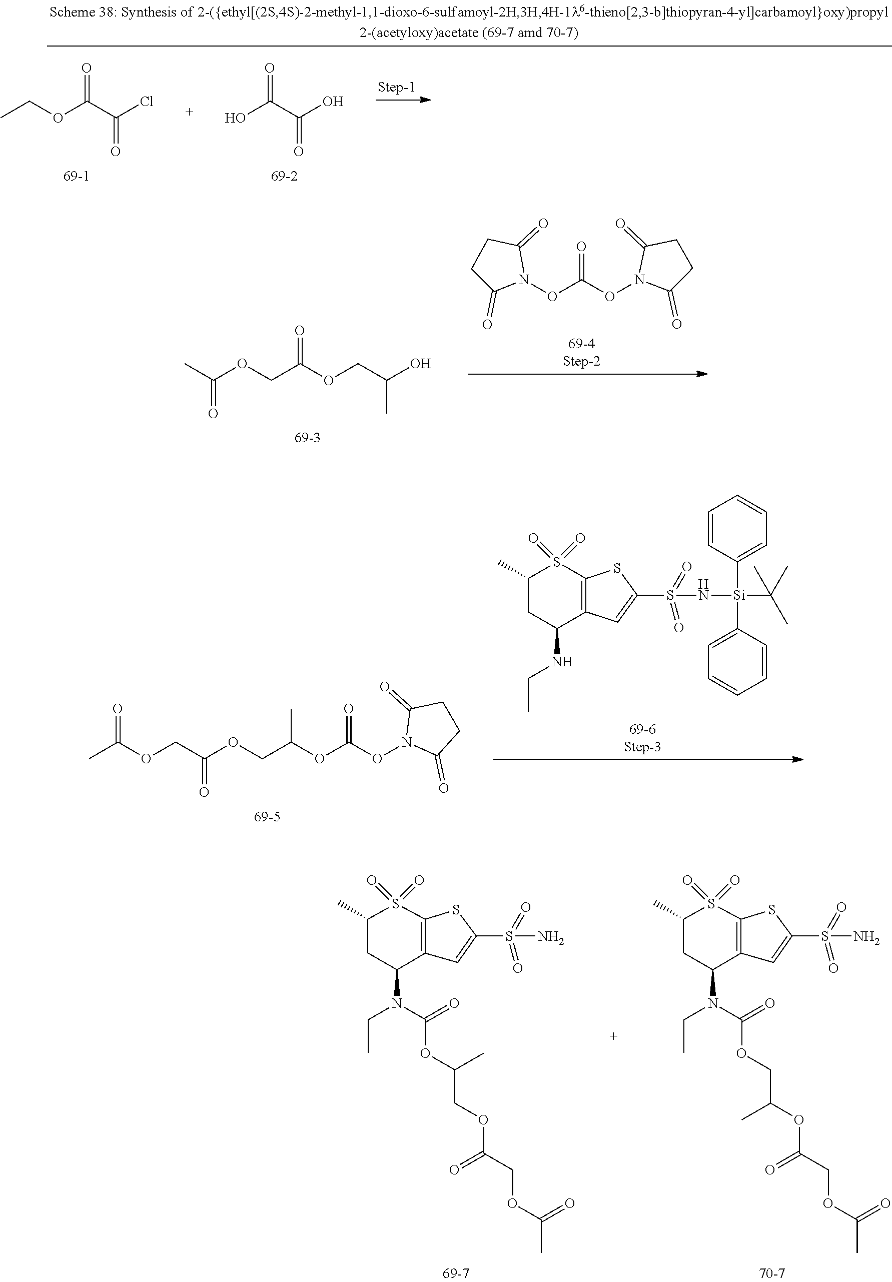

Step-1: Preparation of 2-hydroxypropyl 2-(acetyloxy)acetate (69-3): To a solution of propane-1,2-diol 69-2 (1 mL, 14.70 mmol) in dichloromethane (10 V) were added TEA (2.12 mL, 14.70 mmol) and 2-chloro-2-oxoethyl acetate 69-1 (1 mL, 7.35 mmol) at 0° C. The reaction mixture was allowed to stir at 25-30° C. over a period of 2 h. The resulting reaction mass was quenched with water (100 mL), extracted with dichloromethane (200 mL), dried over sodium sulfate and concentrated under reduced pressure. The crude product was purified by silica gel column chromatography (230-400 mesh) to obtain product 69-3 as a colorless liquid 0.8 g (51.61%).

Step-2: Preparation of 2-({[(2-oxopyrrolidin-1-yl)oxy]carbonyl}oxy)propyl 2-(acetyloxy)acetate (69-5): To a solution of 2-hydroxypropyl 2-(acetyloxy)acetate 69-3 (0.8 g, 4.54 mmol) in THF (10V) were added pyridine (0.74 mL, 9.09 mmol) and bis(2,5-dioxopyrrolidin-1-yl) carbonate 69-4 (3.49 g, 13.63 mmol) at 25-30° C. The reaction mixture was allowed to stir at 25-30° C. over a period of 16 h. The reaction mass was quenched with 1% H₃PO₄ solution (50 mL), extracted with ethyl acetate (100 mL×2), dried over sodium sulfate and concentrated under reduced pressure. The crude product was purified by silica gel column chromatography (230-400 mesh) to obtain product 69-5 as a colorless liquid 0.6 g (41.66%).

Step-3: Preparation of 2-({[(4S)-1,1-dioxo-6-sulfamoyl-2H,3H,4H-1λ⁶-thieno[2,3-b]thiopyran-4-yl](ethyl)carbamoyl}oxy)propyl 2-(acetyloxy)acetate (69-7): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 69-6 (0.5 g, 0.889 mmol) in THF (10V) were added pyridine (0.0725 mL, 0.889 mmol), DMAP (0.021 g, 0.177 mmol) and 2-({[(2,5-dioxopyrrolidin-1-yl)oxy]carbonyl}oxy)propyl 2-(acetyloxy)acetate 69-5 (0.564 g, 1.77 mmol) at 25-30° C. The reaction mixture was allowed to stir at 25-30° C. over a period of 16 h. After completion of the reaction, the reaction was quenched with water (50 mL), extracted with ethyl acetate (100 mL), dried over sodium sulfate and concentrated under reduced pressure. Isolated compound 69-7 and 70-7 in two fractions (Isomer-1 and Isomer-2) by preparative HPLC in 22 mg and 47 mg scale respectively (14.74%). Fractions are composed of differing mixtures of regio- and stereo-isomers of the propylene glycol group. Fraction 1. ¹H NMR (400 MHz, DMSO-d6/TFA) δ 8.06 (bs, 2H), 7.29 (s, 1H), 5.2-4.5, (m, 4H), 4.3-2.6 (m, 6H), 2.5-2.4 (m, 1H), 2.09 and 2.07 (2s, 3H), 1.38 (d, 3H), 1.3-0.6 (m, 6H); m/z [M−H]⁻ 525.1. Fraction 2. ¹H NMR (400 MHz, DMSO-d6) δ 8.0 (bs, 2H), 7.34-7.24 (m, 1H), 5.2-4.5, (m, 4H), 4.35-3.0 (m, 5H), 2.90-2.70 (m, 1H), 2.5-2.4 (m, 1H), 2.13-2.03 (m, 3H), 1.38 (d, 3H), 1.25-0.9 (m, 6H); m/z [M+NH₄]⁺ 544.1.

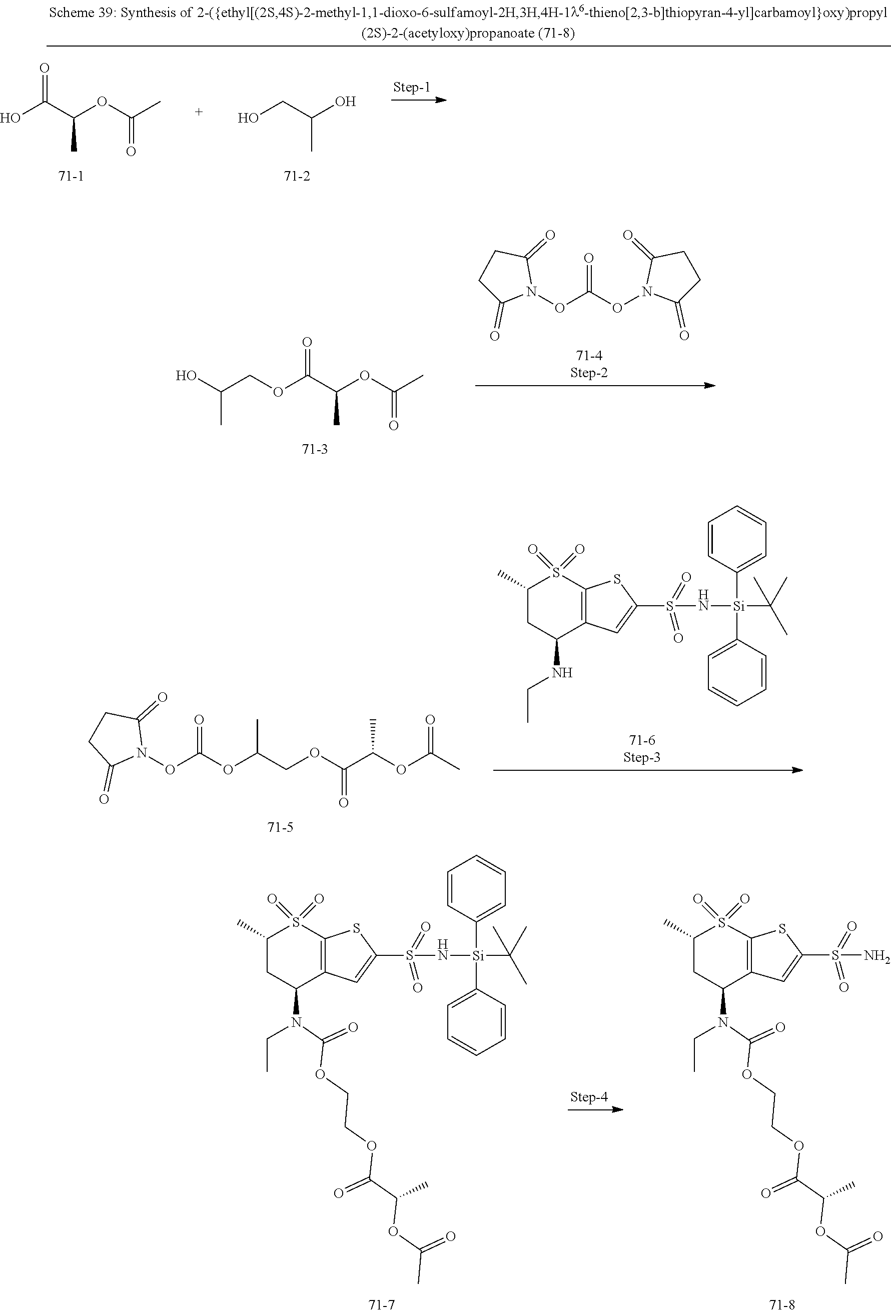

Step-1: Preparation of 2-hydroxypropyl (2S)-2-(acetyloxy)propanoate (71-3): To a solution of (2S)-2-(acetyloxy)propanoic acid 71-1 (1.3 g, 9.85 mmol) in dichloromethane (10 V) were added EDC HCl (1.8 g, 9.85 mmol), propane-1,2-diol 71-2 (0.74 g, 9.85 mmol), and 4-Dimethylaminopyridine (80 mg, 0.65 mmol) at 0° C. The reaction mixture was allowed to stir at 25-30° C. over a period of 2 h. After completion of the reaction, the resulting reaction mass was quenched with water (100 mL) and extracted with ethyl acetate (200 mL), dried over sodium sulfate and concentrated under reduced pressure. The crude product obtained upon evaporation of volatiles was purified by silica gel (230-400 mesh) column chromatography to obtain product 71-3 as a colorless liquid 800 mg (66.6%).

Step-2: Preparation 2-({[(2,5-dioxopyrrolidin-1-yl)oxy]carbonyl}oxy)propyl (2S)-2-(acetyloxy)propanoate (71-5): To a solution of 2-hydroxypropyl (2S)-2-(acetyloxy)propanoate 71-3 (0.8 g, 4.20 mmol) in tetrahydrofuran (10 V) were added pyridine (1.27 mL, 12.6 mmol), bis(2,5-dioxopyrrolidin-1-yl) carbonate 71-4 (2.69 g, 10.52 mmol) and 4-Dimethylaminopyridine (0.1 g, 0.84 mmol) at 0° C. The reaction mixture was allowed to stir at 25-30° C. over a period of 16 h. After completion of the reaction, the resulting reaction mass was quenched with water (50 mL) and extracted with ethyl acetate (100 mL), dried over sodium sulfate and concentrated under reduced pressure. The crude product obtained upon evaporation of volatiles was purified by silica gel (230-400 mesh) column chromatography to obtain product 71-5 as a colorless wax 900 mg (69.2%).

Step-3: Preparation 2-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)propyl (2S)-2-(acetyloxy)propanoate (71-7): To a solution of (2-({[(2,5-dioxopyrrolidin-1-yl)oxy]carbonyl}oxy)propyl (2S)-2-(acetyloxy)propanoate 71-6 (0.5 g, 0.88 mmol) in tetrahydrofuran (10 V) were added pyridine (0.18 mL, 1.77 mmol), 2-({[(2,5-dioxopyrrolidin-1-yl)oxy]carbonyl}oxy)propyl (2S)-2-(acetyloxy)propanoate 71-5 and 4-Dimethylaminopyridine (21 mg, 0.177 mmol) at 0° C. The reaction mixture was allowed to stir at 25-30° C. over a period of 24 h. After completion of the reaction, the resulting reaction mass was quenched with water (100 mL) and extracted with ethyl acetate (200 mL), dried over sodium sulfate and concentrated under reduced pressure to obtain crude product 71-7 as an off white solid (690 mg). The crude compound was carried as such into next step without any purification.

Step-4: Preparation of 2-({ethyl[(2S,4S)-2-methyl-1,1-dioxo-6-sulfamoyl-2H,3H,4H-1λ⁶-thieno[2,3-b]thiopyran-4-yl]carbamoyl}oxy)propyl (2S)-2-(acetyloxy)propanoate (71-8): To a solution of 2-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)propyl (2S)-2-(acetyloxy)propanoate 71-7 (0.69 g, 0.88 mmol) in tetrahydrofuran (10 V) were added acetic acid (0.014 mL, 0.26 mmol) and TBAF (0.26 mL, 0.26 mmol) at 0° C. The reaction mixture was allowed to stir at 0° C. over a period of 30 min. After completion of the reaction, the resulting reaction mass was quenched with water (100 mL) and extracted with ethyl acetate (200 mL), dried over sodium sulfate and concentrated under reduced pressure. The crude compound was purified by preparative HPLC to obtain compound 71-8 as a mixture of regio- and stereo-isomers at the propylene glycol group as a white solid 150 mg (31.9%). ¹H NMR (400 MHz, DMSO-d6) δ 8.08 (bs, 2H), 7.28 (s, 1H), 5.2-4.7, (m, 3H), 4.4-3.7 (m, 3H), 3.6-2.7 (m, 3H), 2.5-2.4 (m, 1H), 2.06 and 2.04 (2s, 3H), 1.45-1.3 (m, 6H), 1.3-0.6 (m, 6H); m/z [M+H]⁺ 541.1.

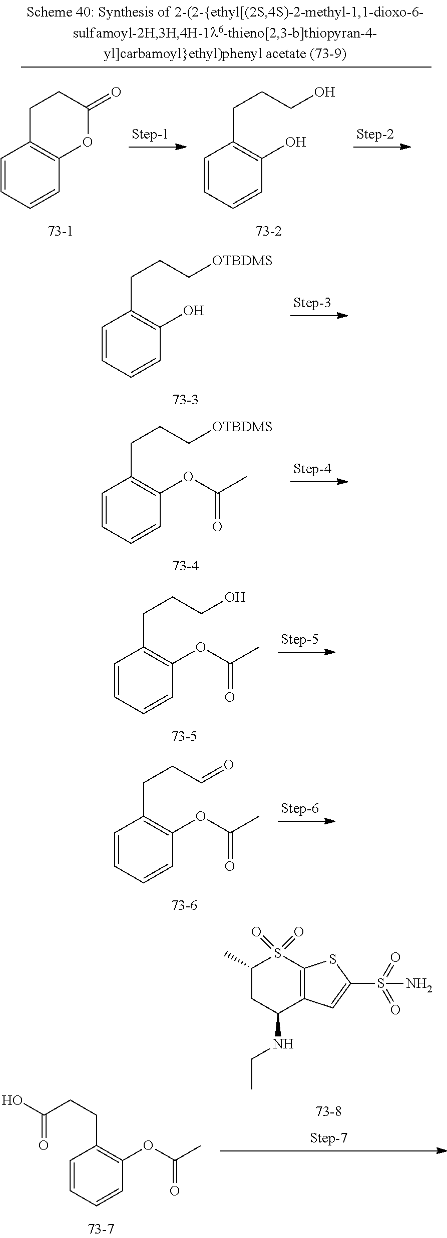

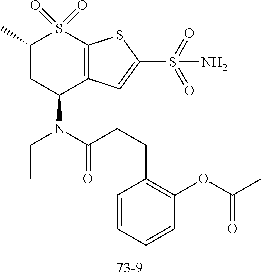 [SMILES: CCN(C(=O)CCc1ccccc1OC(C)=O)[C@H]1C[C@H](C)S(=O)(=O)c2sc(S(N)(=O)=O)cc21]

Step-1: Preparation of 2-(3-hydroxypropyl)phenol (73-2): To a solution of 3,4-dihydro-2H-1-benzopyran-2-one 73-1 (10.0 g, 67.56 mmol) in tetrahydrofuran (25 V) was added LAH (3.84 g, 101.3 mmol) at 0-5° C. The reaction mixture was allowed to stir at 0-5° C. over a period of 1 h. The resulting reaction mass was quenched with ammonium chloride solution (400 mL), extracted with ethyl acetate (2×750 mL), organic layer was dried over sodium sulfate and concentrated under reduced pressure to obtain compound 73-3 as a colorless liquid 9.2 g (83%) . The crude compound was taken forward to next step without any purification.

Step-2: Preparation of 2-{3-[(tert-butyldimethylsilyl)oxy]propyl}phenol (73-3): To a solution of 2-(3-hydroxypropyl)phenol 73-2 (9.2 g, 34.52 mmol) in N,N-dimethylformamide (3 V) was added imidazole (3.53 g, 51.87 mmol)) and TBDMSCl (3.84 g, 51.79 mmol) at 0-5° C. The reaction mixture was allowed to stir at room temperature over a period of 2 h. The resulting reaction mass was quenched with water (200 mL), extracted with dichloromethane (2×250 mL), organic layer was dried over sodium sulphate and concentrated under reduced pressure. The crude compound was purified by silica gel (60-120) column chromatography to obtain product 73-3 as a colorless liquid 10.5 g (64%).

Step-3: Preparation of 2-{3-[(tert-butyldimethylsilyl)oxy]propyl}phenyl acetate (73-4): To a solution of 2-{3-[(tert-butyldimethylsilyl)oxy]propyl}phenol 73-3 (5.5 g, 20.64 mmol) in dicloromethane (3 V) were added triethylamine(3.53 g, 51.87 mmol)) and N,N-dimethylaminopyridine (29.05 g, 206.7 mmol) at 24-25° C. Followed by acetic anhydride(15.63 g, 165.4 mmol) at at 0-5° C. The reaction mixture was allowed to stir at room temperature over a period of 3 h. The resulting reaction mass was quenched with water (200 mL), extracted with ethylacetate (2×250 mL), organic layer was dried over sodium sulphate and concentrated under reduced pressure to obtain product 73-4 as an colorless wax 5.0 g (78%). The crude compound was as taken into next step.

Step-4: Preparation of 2-(3-hydroxypropyl)phenyl acetate (73-5): To a solution of 2-{3-[(tert-butyldimethylsilyl)oxy]propyl}phenyl acetate 73-4 (5.0 g, 16.20 mmol) in tetrahydrofuran (2 V) were added water(10 mL, 2V) and acetic acid (30 mL, 6V) at 24-25° C. The reaction mixture was allowed to stir at room temperature over a period of 3 h at 24-25° C. The resulting reaction mass was quenched with water (200 mL), extracted with ethyl acetate (2×250 mL), organic layer was dried over sodium sulphate and concentrated under reduced pressure to obtain product 73-5 as a colorless liquid 1.8 g (57%).

Step-5: Preparation of 2-(3-oxopropyl)phenyl acetate (73-6): To a solution of 2-(3-hydroxypropyl)phenyl acetate 73-5 (1.8 g, 9.27 mmol) in dichloromethane (5 V) was added pyridinium chlorochromate (2.0 g, 20.85 mmol) at 24-25° C. The reaction mixture was allowed to stir at room temperature over a period of 2 h at 24-25° C. The resulting reaction mass was quenched with water (80 mL), extracted with ethyl acetate (2×100 mL), organic layer was dried over sodium sulphate and concentrated under reduced pressure. The crude compound was purified through silica gel (230-400 mesh) column chromatography to obtain product 73-6 as a colorless oil 1.3 g (73%).

Step-6: Preparation of 3-[2-(acetyloxy)phenyl]propanoic acid (73- 7):To a solution of 2-(3-oxopropyl)phenyl acetate 73-6 (3.1 g, 7.90 mmol) in tertiary butanol (20 V), was added 2-methyl butane (12.71 mL, 4.1 V). After 10 min, sodium chlorite (37.13 g, 1.46 mmol) and sodium dihydrogen phosphate (9.92 mL, 3.2 V, 0.67 M) were added at 25-28° C. The reaction mixture was allowed to stir at 25-28° C. over a period of 1 h. The resulting reaction mass was quenched with water (200 mL), extracted with ethyl acetate (500 mL×2), dried over sodium sulfate and concentrated under reduced pressure. The crude product obtained upon evaporation of volatiles was purified through silica gel (230-400 mesh) column chromatography to obtain product 73-7 as an off-white solid, 2.5 g (74.4%).

Step-7: Preparation of 2-(2-{ethyl[(2S,4S)-2-methyl-1,1-dioxo-6-sulfamoyl-2H,3H,4H-1λ⁶-thieno[2,3-b]thiopyran-4-yl]carbamoyl}ethyl)phenyl acetate (73-9): To a solution of 3-[2-(acetyloxy)phenyl]propanoic acid 73-7 (1.86 g, 8.96 mmol) in dichloromethane (20 mL), were added oxalyl chloride (2.29 mL, 26.93 mmol) and N,N-dimethylformamide (0.01 mL) at 0° C. The reaction mixture was allowed to stir at 0-5° C. over a period of 30 min. After completion of reaction, the reaction mixture was concentrated to dryness under nitrogen atmosphere. The residue was diluted with dichloromethane (10 V) and added to dorzolamide 73-8 (2.3 g, 6.40 mmol) neutralized with N,N-diisopropylethylamine (2.32 ml, 12.81 mmol) in dichloromethane (5 V) at 0° C. The reaction mixture was allowed to stir at 25-30° C. over a period of 1 h. The resulting reaction mass was quenched with water (100 mL), extracted with ethyl acetate (250 mL×2), dried over sodium sulfate and concentrated under reduced pressure. The crude was further purified by reverse phase column chromatography to obtain product 73-9 as an off-white solid, 0.5 g (15%). ¹H NMR (400 MHz, DMSO-d6) δ 8.06 (bs, 2H), 7.42-7.30 (m, 1H), 7.30-7.16 (m, 3H), 7.09-6.96 (m, 1H), 5.37-5.0, (m, 1H), 3.97-3.82 (m, 1H), 3.5-3.1 (m, 2H), 2.9-2.5 (m, 5H), 2.42-2.28 (m, 1H), 2.29 (s, 3H), 1.44-1.31 (m, 3H), 1.16-0.95 (m, 3H); m/z [M+H]⁺ 515.4.

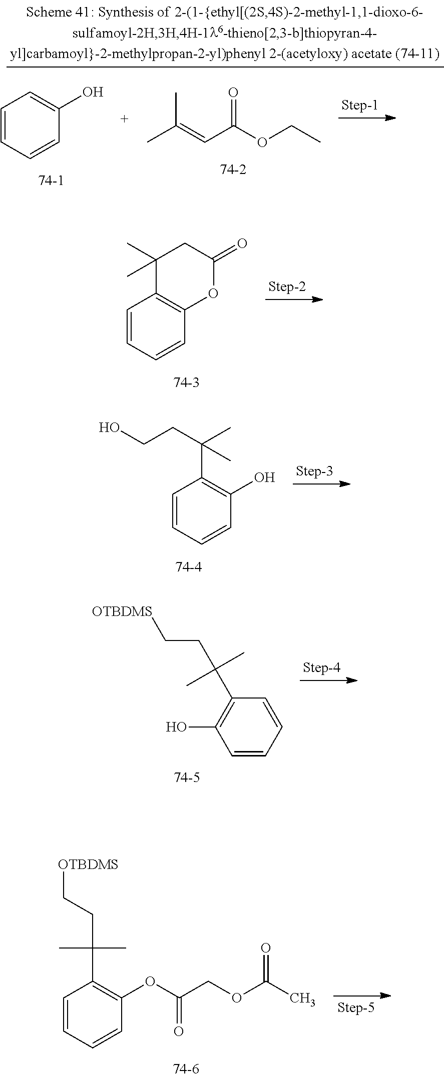

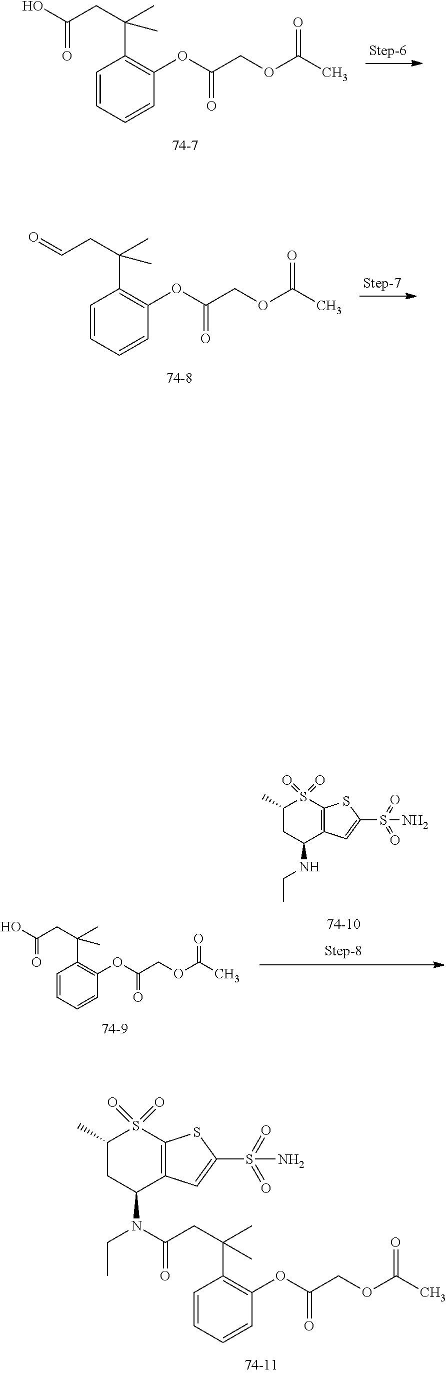

Step-1: Preparation of 4,4-dimethyl-3,4-dihydro-2H-1-benzopyran-2-one (74-3): To a solution of phenol 74-1 (5.0 g, 4.99 mmol) in methane sulfonic acid (4 V) was added ethyl 3-methylbut-2-enoate 74-2 (6.39 g, 4.9 mmol) at 25-28° C. The reaction mixture was allowed to stir at 70° C. over a period of 2 h. The resulting reaction mass was quenched with water (100 mL), extracted with ethyl acetate (250 mL×2), dried over sodium sulfate and concentrated under reduced pressure. The crude product obtained upon evaporation of volatiles was purified through silica gel (230-400 mesh) column chromatography (1-3% ethyl acetate/ hexanes) to obtain product 74-3 as a colorless oil, 3.7 g (41.7%).

Step-2: Preparation of 2-(4-hydroxy-2-methylbutan-2-yl)phenol (74-4): To a solution of lithium aluminium hydride (0.097 g, 0.25 mmol) in dry tetrahydrofuran (5 V) was added 4,4-dimethyl-3,4-dihydro-2H-1-benzopyran-2-one 74-3 (3.7 g, 9.8 mmol) at 0° C. The reaction mixture was allowed to stir at 25-28° C. over a period of 1 h. The resulting reaction mass was quenched with 1.5 N HCl (20 mL), extracted with ethyl acetate (70 mL×2), dried over sodium sulfate and concentrated under reduced pressure. The crude product 74-4 obtained upon evaporation of volatiles was taken forward to next step 3.03 g (82%)

Step-3: Preparation of 2-{4-[(tert-butyldiphenylsilyl)oxy]-2-methylbutan-2-yl}phenol (74-5): To a solution of 2-(4-hydroxy-2-methylbutan-2-yl)phenol 74-4 (0.30 g, 1.66 mmol) in N,N-dimethyl formamide (5 V), tertiarybutyldimethylsilyl chloride (0.37 g, 2.49 mmol) and imidazole (0.16 g, 2.4 mmol) were added at 0° C. The reaction mixture was allowed to stir at 25-30° C. over a period of 1 h. The resulting reaction mass was quenched with water (50 mL), extracted with ethyl acetate (100 mL), dried over sodium sulfate and concentrated under reduced pressure. The crude product 74-5 obtained upon evaporation of volatiles was taken forward to next step 0.39 g (79%).

Step-4: Preparation of 2-{4-[(tert-butyldiphenylsilyl)oxy]-2-methylbutan-2-yl}phenyl 2-(acetyloxy)acetate (74-6): To a solution of 2-{4-[(tert-butyldiphenylsilyl)oxy]-2-methylbutan-2-yl}phenol 74-5 (7.0 g, 16.7 mmol) in dichloromethane (10 V), N,N-diisopropylethylamine (7.8 mL, mmol) and acetoxyacetyl chloride (1.8 mL, 16.7 mmol) were added at 0° C. The reaction mixture was then allowed to stir at 25-28° C. over a period of 3 h. The resulting reaction mass was quenched with water (200 mL), extracted with ethyl acetate (150 mL×2), dried over sodium sulfate and concentrated under reduced pressure. The crude product 74-6 obtained upon evaporation of volatiles was taken forward to next step 7.0 g (Crude compound).

Step-5: Preparation of 2-(4-hydroxy-2-methylbutan-2-yl)phenyl 2-(acetyloxy)acetate (74-7): To a solution of 2-{4-[(tert-butyldiphenylsilyl)oxy]-2-methylbutan-2-yl}phenyl 2-(acetyloxy)acetate 74-6 (7.0 g, 13.5 mmol) in tetrahydrofuran (14 mL, 2 V) were added acetic acid (42 mL, 6 V) and water (14 mL, 2 V) at 0° C. The reaction mixture was allowed to stir at 25-28° C. over a period of 3 h. The resulting reaction mass was quenched with water (500 mL), extracted with ethyl acetate (250 mL×2), dried over sodium sulfate and concentrated under reduced pressure. The crude product obtained upon evaporation of volatiles was purified by silica gel (230-400 mesh) column chromatography (10% ethyl acetate in hexanes) to obtain product 74-7 as a colorless oil, 3.0 g (79%).

Step-6: Preparation of 2-(2-methyl-4-oxobutan-2-yl)phenyl 2-(acetyloxy)acetate (74-8): To a solution 2-(4-hydroxy-2-methylbutan-2-yl)phenyl 2-(acetyloxy)acetate 74-7 (4 g, 14.28 mmol) in dichloromethane (10 V), was added pyridinium chlorochromate (6.9 g, 32.14 mmol) at 0° C. The reaction mixture was allowed to stir at 25-28° C. over a period of 2 h. The resulting reaction mass was diluted with water (200 mL), extracted with ethyl acetate (150 mL×2), dried over sodium sulfate and concentrated under reduced pressure. The crude product obtained upon evaporation of volatiles was purified through silica gel (230-400 mesh) column chromatography (12% ethyl acetate in hexanes) to obtain product 74-8 as a colorless oil, 2.5 g (62%).

Step-7: Preparation of 3-(2-{[2-(acetyloxy)acetyl]oxy}phenyl)-3-methylbutanoic acid (74-9): To a solution of 2-(2-methyl-4-oxobutan-2-yl)phenyl 2-(acetyloxy)acetate 74-8 (2.5 g, 8.99 mmol) in tertiary butanol (50 mL, 20 V), was added 2-methyl butane (10.25 mL, 4.1 V). After 10 minutes (1.87 g, 20.68 mmol) and sodium dihydrogen phosphate (8 mL, 3.2 V, 0.67 M) were added at 25-28° C. The reaction mixture was allowed to stir at 25-28° C. over a period of 1 h. The resulting reaction mass was quenched with water (200 mL), extracted with ethyl acetate (150 mL×2), dried over sodium sulfate and concentrated under reduced pressure. The crude product obtained upon evaporation of volatiles was purified through silica gel (230-400 mesh) column chromatography (15% ethyl acetate in hexanes) to obtain product 74-9 as an off-white solid, 1.5 g (56%).

Step-8: Preparation of 2-(1-{ethyl[(2S,4S)-2-methyl-1,1-dioxo-6-sulfamoyl-2H,3H,4H-1λ⁶-thieno[2,3-b]thiopyran-4-yl]carbamoyl}-2-methylpropan-2-yl)phenyl 2-(acetyloxy)acetate (74-11): To a solution of 3-(2-{[2-(acetyloxy)acetyl]oxy}phenyl)-3-methylbutanoic acid 74-9 (1.9 g, 6.48 mmol) in dichloromethane (20 mL), were added oxalyl chloride (1.18 mL, 13.8 mmol) and N,N-dimethylformamide (0.001 ml) at 0° C. The reaction mixture was allowed to stir at 25-30° C. over a period of 30 min. After completion of reaction, the reaction mixture was concentrated to dryness under nitrogen atmosphere, diluted with dichloromethane (5 V) and added to dorzolamide 74-10 (1.5 g, 4.62 mmol) neutralized using N,N-diisopropylethylamine (1.6 ml, 9.25 mmol) in dichloromethane (5 V) at 0° C. The reaction mixture was allowed to stir at 25-30° C. over a period of 1 h. The resulting reaction mass was quenched with water (200 mL), extracted with ethyl acetate (200 mL×2), dried over sodium sulfate and concentrated under reduced pressure. The crude compound was purified by reverse phase column chromatography to obtain product 74-11 as white solid, 0.35 g (12%). ¹H NMR (400 MHz, DMSO-d6/TFA) δ 8.09 and 8.06 (2bs, 2H), 7.45-7.30 (m, 1H), 7.28-7.04 (m, 3H), 6.99 (d, 1H), 5.35-5.05, (m, 1H), 5.04-4.92 (m, 2H), 3.94-3.76 (m, 1H), 3.5-2.5 (m, 5H), 2.36-2.20 (m, 1H), 2.14, 2.07 and 2.06 (3s, 3H), 1.49-1.24 (m, 9H), 1.12 and 0.84 (2t, 3H); m/z [M+H]⁺ 601.4.

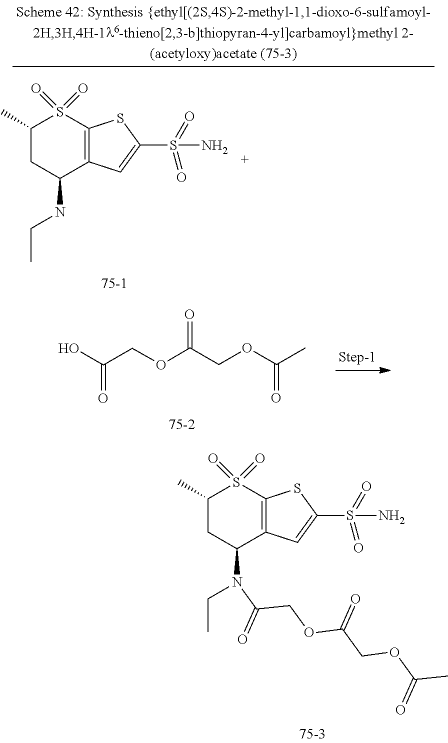 [SMILES: CCN[C@H]1C[C@H](C)S(=O)(=O)c2sc(S(N)(=O)=O)cc21 ; CC(=O)OCC(=O)OCC(=O)O ; CCN(C(=O)COC(=O)COC(C)=O)[C@H]1C[C@H](C)S(=O)(=O)c2sc(S(N)(=O)=O)cc21]

To a solution of 2-{[2-(acetyloxy)acetyl]oxy}acetic acid 75-2 (0.88 g, 5.01 mmol) in dichloromethane (20 mL), were added oxalyl chloride (0.85 mL, 9.99 mmol) and N,N-dimethylformamide (0.05 mL) at 0° C. The reaction mixture was allowed to stir at room temperature over a period of 30 min and concentrated to dryness under nitrogen atmosphere. The residue was diluted with dichloromethane (100 mL) and added N,N-diisopropylethylamine (0.25 mL, 1.41 mmol) followed by Dorzolamide 75-1 (1.2 g, 3.34 mmol) at 0° C. The reaction mixture was allowed to stir at room temperature over a period of 2 h. The resulting reaction mass was quenched with water (30 mL), extracted with dichloromethane (2×100 mL), organic layer was dried over sodium sulphate and concentrated under reduced pressure. The crude product obtained upon evaporation of volatiles was purified through silica gel (230-400 mesh) column chromatography to obtain product 75-3 as an off white solid 0.18 g (11%). ¹H NMR (400 MHz, DMSO-d6) δ 8.11 and 8.05 (2bs, 2H), 7.41 and 7.28 (2s, 1H), 5.35-4.69 (m, 5H), 3.97-3.85 (m, 1H), 3.49-3.10 (m, 2H), 2.91-2.70 (m, 1H), 2.45-2.30 (m, 1H), 2.11 and 2.10 (2s, 3H), 1.43 and 1.37 (2d, 3H), 1.18 and 0.98 (2t, 3H); m/z [M+H]⁺ 483.2.

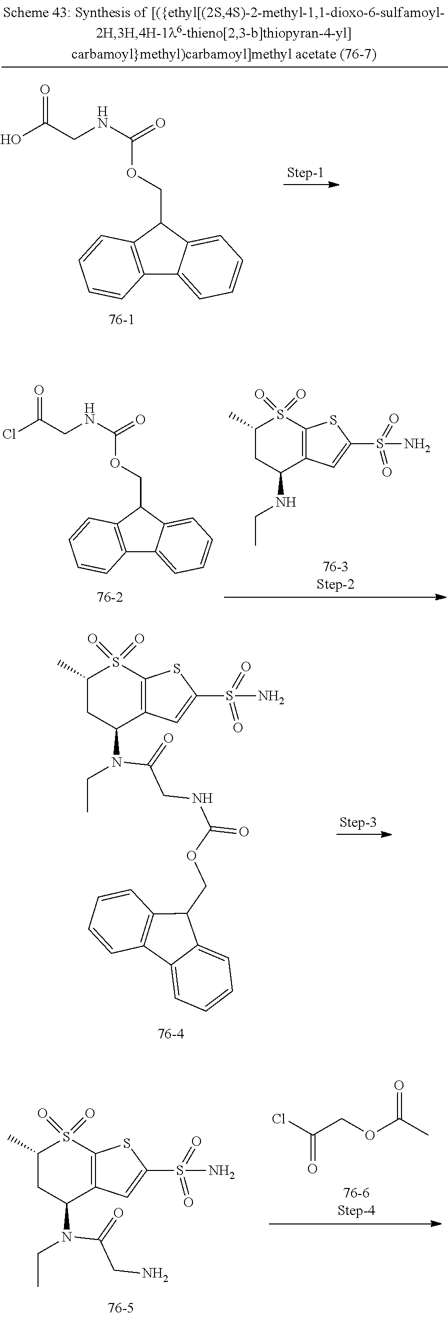

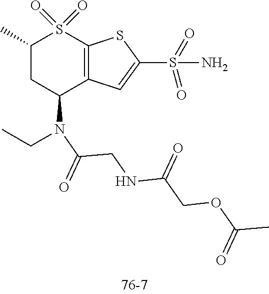 [SMILES: CCN(C(=O)CNC(=O)COC(C)=O)[C@H]1C[C@H](C)S(=O)(=O)c2sc(S(N)(=O)=O)cc21]

Step-1: Preparation of (9H-fluoren-9-yl)methyl (2-chloro-2-oxoethyl)carbamate (76-2): To a solution of ({[(9H-fluoren-9-yl)methoxy]carbonyl}amino)acetic acid 76-1 (10.0 g, 6.71 mmol) in dichloromethane (8 V) and tetrahydrofuran (2.0 V) was added thionylchloride (1.94 mL, 26.8 mmol) at 0° C. The reaction was heated to 75° C. for 2 h. The reaction mass was cooled to 25-28° C. The resulting reaction mass was diluted with ethyl acetate (500 mL), washed with water (250 mL×2), organic layer was dried over sodium sulfate and concentrated under reduced pressure to obtain compound 76-2 as an off white solid 6.0 g (47%). The crude compound was taken forward to next step without any purification.

Step-2: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate (76-4): To a solution of dorzolamide 76-3 (1.0 g, 2.77 mmol) in dichloromethane (10 V) was added N,N-Diisopropylethylamine (1.0 mL, 5.5 mmol) at 0° C. After 30 min, was added 9H-fluoren-9-yl)methyl (2-chloro-2-oxoethyl)carbamate 76-2 (1.31 g, 4.1 mmol) at 0° C. The reaction mixture was allowed to stir at 25-30° C. over a period of 1 h. The resulting reaction mass was diluted with ethyl acetate (150 mL) and washed with water (50 mL×2), organic layer was dried over sodium sulfate and concentrated under reduced pressure to obtain compound 76-4 as an off white solid 0.67 g (40%). The crude compound was taken forward to next step without any purification.

Step-3: Preparation of 2-amino-N-ethyl-N-[(4S,6S)-6-methyl-7,7-dioxo-2-sulfamoyl-4,5,6,7-tetrahydro-7l6-thieno[2,3-b]thiopyran-4-yl]acetamide (76-5): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate 76-4 (0.3 g, 0.49 mmol) in dichloromethane (5 V) was added piperidine (0.30 mL, 2.48 mmol) at 0° C. The reaction mixture was allowed to stir at 25-30° C. over a period of 24 hours. The resulting reaction mixture was concentrated under reduced pressure. The crude compound was purified by reverse phase column chromatography to obtain product 76-5 as a white solid 0.18 g (13%).

Step-4: Preparation of [({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamoyl]methyl acetate (76-7): To a solution of 2-amino-N-ethyl-N-[(4S,6S)-6-methyl-7,7-dioxo-2-sulfamoyl-4,5,6,7-tetrahydro-7l6-thieno[2,3-b]thiopyran-4-yl]acetamide 76-5 (0.1 g, 0.26 mmol) in dichloromethane (10 mL) were added N,N-Diisopropylethylamine (0.14 mL, 0.78 mmol) and acetoxyacetyl chloride 76-6 (0.02 mL g, 0.23 mmol) at 0° C. The reaction mixture was allowed to stir at 25-30° C. over a period of 16 h. The resulting reaction mass was quenched with water (50 mL), extracted with ethyl acetate (100×2 mL), dried over sodium sulphate and concentrated under reduced pressure. The crude compound was purified by preparative HPLC to obtain product 76-7 as a white solid 0.05 g (39%). ¹H NMR (400 MHz, DMSO-d6) δ 8.27 and 8.19 (2t, 1H), 8.04 (bs, 2H), 7.41 and 7.28 (2s, 1H), 5.35-4.95 (m, 1H), 4.53 and 4.49 (2s, 2H), 4.16-3.85 (m, 3H), 3.53-3.10 (m, 2H), 2.88-2.60 (m, 1H), 2.45-2.30 (m, 1H), 2.10 and 2.08 (2s, 3H), 1.43 and 1.37 (2d, 3H), 1.20 and 1.01 (2t, 3H); m/z [M+H]⁺ 482.3.

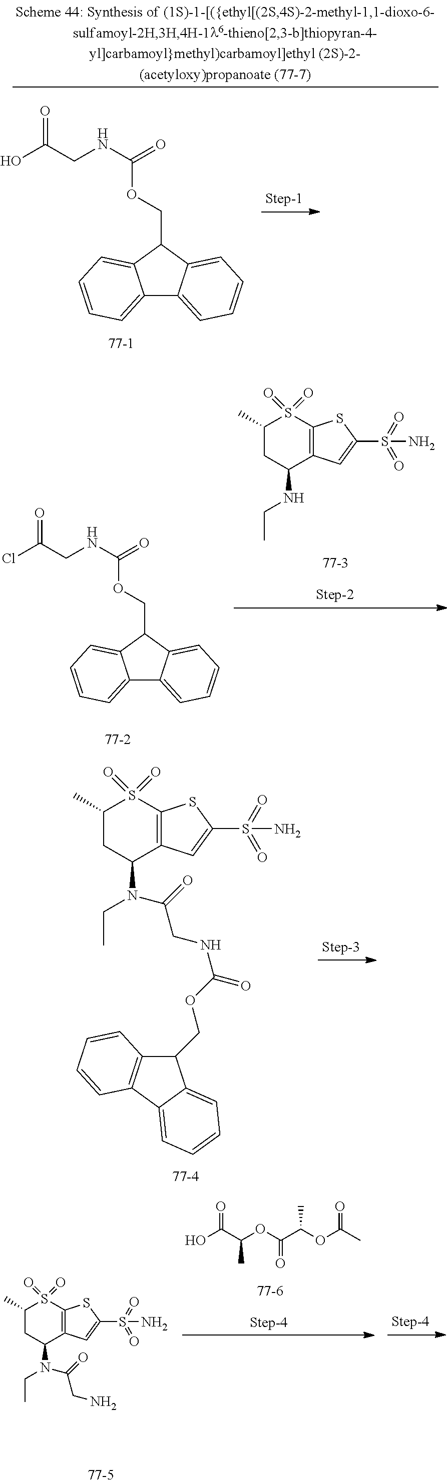 [SMILES: O=C(O)CNC(=O)OCC1c2ccccc2-c2ccccc21 ; O=C(Cl)CNC(=O)OCC1c2ccccc2-c2ccccc21 ; CCN[C@H]1C[C@H](C)S(=O)(=O)c2sc(S(N)(=O)=O)cc21 ; CCN(C(=O)CNC(=O)OCC1c2ccccc2-c2ccccc21)[C@H]1C[C@H](C)S(=O)(=O)c2sc(S(N)(=O)=O)cc21 ; CC(=O)O[C@@H](C)C(=O)O[C@@H](C)C(=O)O ; CCN(C(=O)CN)[C@H]1C[C@H](C)S(=O)(=O)c2sc(S(N)(=O)=O)cc21]

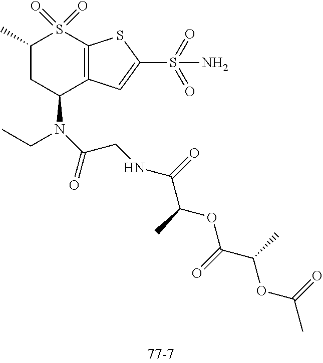 [SMILES: CCN(C(=O)CNC(=O)[C@H](C)OC(=O)[C@H](C)OC(C)=O)[C@H]1C[C@H](C)S(=O)(=O)c2sc(S(N)(=O)=O)cc21]

Step-1 & 2: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate (77-4): To a solution of ({[(9H-fluoren-9-yl)methoxy]carbonyl}amino)acetic acid 77-1 (1.2 g, 4.1 mmol) in dichloromethane (20 mL), were added oxalyl chloride (0.7 mL, 8.2 mmol) and N,N-dimethylformamide (0.1 mL) at 0° C. The reaction mixture was allowed to stir at room temperature over a period of 30 min and concentrated the reaction mixture to dryness under nitrogen atmosphere. The residue was diluted with dichloromethane (100 mL), added N,N-diisopropylethylamine (0.97 mL, 5.5 mmol) followed by dorzolamide 77-3 (1.0 g, 2.7 mmol) at 0° C. The reaction mixture was allowed to stir at room temperature over a period of 1 h. The resulting reaction mass was quenched with water (50 mL), extracted with dichloromethane (2×100 mL), organic layer was dried over sodium sulphate and concentrated under reduced pressure. The crude product obtained upon evaporation of volatiles was purified by silica gel column (230-400 mesh) chromatography to obtain product 77-4 as a white solid 0.56 g (35%).

Step-3: Preparation of 2-amino-N-ethyl-N-[(4S,6S)-6-methyl-7,7-dioxo-2-sulfamoyl-4,5,6,7-tetrahydro-7l6-thieno[2,3-b]thiopyran-4-yl]acetamide (77-5): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate 77-4 (0.43 g, 0.0074 mmol) in dichloromethane (5 V) was added piperidine (0.39 mL, 3.7 mmol) at 0° C. The reaction mixture was allowed to stir at 25-30° C. over a period of 24 hours. The resulting reaction mixture was as such concentrated under reduced pressure to obtain compound 77-5 as an off white solid 0.25 g (40%). The crude compound was taken forward to next step without any purification.

Step-4: Preparation of (1S)-1-[({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamoyl]ethyl (2S)-2-(acetyloxy)propanoate (77-7): To a solution of (2S)-2-{[(2S)-2-(acetyloxy)propanoyl]oxy}propanoic acid 77-6 (0.21 g, 1.06 mmol) in dichloromethane (20 mL), were added oxalyl chloride (0.18 mL, 2.11 mmol) and N,N-dimethylformamide (0.05 mL) at 0° C. The reaction mixture was allowed to stir at room temperature over a period of 30 min and concentrated to dryness under nitrogen atmosphere. The residue was diluted with dichloromethane (100 mL), and added N,N-diisopropylethylamine (0.25 mL, 1.41 mmol) followed by 2-amino-N-ethyl-N-[(2S,4S)-2-methyl-1,1-dioxo-6-sulfamoyl-2H,3H,4H-1λ⁶-thieno[2,3-b]thiopyran-4-yl]acetamide 77-5 (0.27 g, 0.70 mmol) at 0° C. The reaction mixture was allowed to stir at room temperature over a period of 30 minutes. The resulting reaction mass was quenched with water (30 mL), extracted with dichloromethane (2×100 mL), organic layer was dried over sodium sulphate and concentrated under reduced pressure. The crude product obtained upon evaporation of volatiles was purified by preparative HPLC to obtain product 77-7 as a white solid 0.075 g (18%). ¹H NMR (400 MHz, DMSO-d6) δ 8.24-8.11 (m, 1H), 8.07 and 8.03 (2bs, 2H), 7.42 and 7.28 (2s, 1H), 5.31-4.93 (m, 3H), 4.13-3.85 (m, 3H), 3.52-3.10 (m, 2H), 2.87-2.60 (m, 1H), 2.5-2.34 (m, 1H), 2.07 and 2.05 (2s, 3H), 1.54-1.27 (m, 9H), 1.20 and 1.01 (2t, 3H); m/z [M+H]⁺ 568.3.

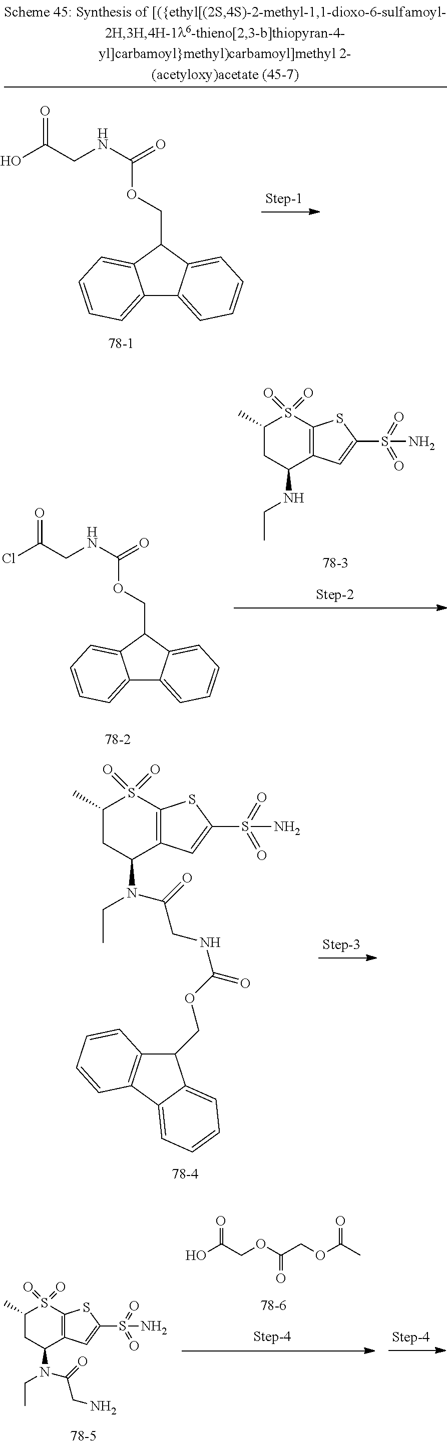 [SMILES: O=C(O)CNC(=O)OCC1c2ccccc2-c2ccccc21 ; O=C(Cl)CNC(=O)OCC1c2ccccc2-c2ccccc21 ; CCN[C@H]1C[C@H](C)S(=O)(=O)c2sc(S(N)(=O)=O)cc21 ; CCN(C(=O)CNC(=O)OCC1c2ccccc2-c2ccccc21)[C@H]1C[C@H](C)S(=O)(=O)c2sc(S(N)(=O)=O)cc21 ; CC(=O)OCC(=O)OCC(=O)O ; CCN(C(=O)CN)[C@H]1C[C@H](C)S(=O)(=O)c2sc(S(N)(=O)=O)cc21]

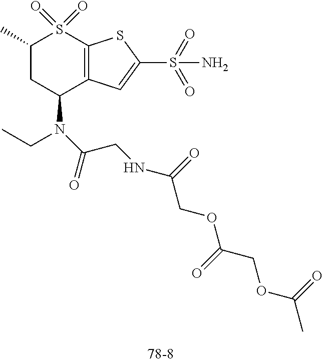 [SMILES: CCN(C(=O)CNC(=O)COC(=O)COC(C)=O)[C@H]1C[C@H](C)S(=O)(=O)c2sc(S(N)(=O)=O)cc21]

Step-1 & 2: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate (78-4): To a solution of ({[(9H-fluoren-9-yl)methoxy]carbonyl}amino)acetic acid 78-1 (1.2 g, 4.1 mmol) in dichloromethane (20 mL), were added oxalyl chloride (0.7 mL, 8.2 mmol) and N,N-dimethylformamide (0.1 mL) at 0° C. The reaction mixture was allowed to stir at room temperature over a period of 30 min and concentrated the reaction mixture to dryness under nitrogen atmosphere. The residue was diluted with dichloromethane (100 mL) and added N,N-diisopropylethylamine (0.97 mL, 5.5 mmol) followed by dorzolamide 78-3 (1.0 g, 2.7 mmol) at 0° C. The reaction mixture was allowed to stir at room temperature over a period of 1 h. The resulting reaction mass was quenched with water (50 mL), extracted with dichloromethane (2×100 mL), organic layer was dried over sodium sulphate and concentrated under reduced pressure. The crude product obtained upon evaporation of volatiles was purified by silica gel column chromatography to obtain product 78-4 as a white solid 0.56 g (35%).

Step-3: Preparation of 2-amino-N-ethyl-N-[(4S,6S)-6-methyl-7,7-dioxo-2-sulfamoyl-4,5,6,7-tetrahydro-7l6-thieno[2,3-b]thiopyran-4-yl]acetamide (78-5): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate 78-4 (0.43 g, 0.0074 mmol) in dichloromethane (5 V) was added piperidine (0.39 mL, 3.7 mmol) at 0° C. The reaction mixture was allowed to stir at 25-30° C. over a period of 24 hours. The resulting reaction mixture was concentrated under reduced pressure to obtain compound 78-5 as an off white solid 0.25 g (40%). The crude compound was taken forward to next step without any purification.

Step-4: Preparation of [({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamoyl]methyl 2-(acetyloxy)acetate (78-7): To a solution of {[(acetyloxy)acetyl]oxy}acetic acid 78-6 (0.13 g, 0.78 mmol) in dichloromethane (10 mL) were added EDC.HCl (0.15 g, 0.78 mmol), N,N-Diisopropylethylamine (0.41 mL, 0.78 mmol) and 2-amino-N-ethyl-N-[(2S,4S)-2-methyl-1,1-dioxo-6-sulfamoyl-2H,3H,4H-1λ⁶-thieno[2,3-b]thiopyran-4-yl]acetamide 78-5 (0.3 g, 0.78 mmol) at 0° C. The reaction mixture was allowed to stir at 25-30° C. over a period of 16 h. The resulting reaction mass was quenched with water (50 mL), extracted with ethyl acetate (100×2 mL), dried over sodium sulphate and concentrated under reduced pressure. The crude compound was purified by reverse phase column chromatography to obtain product 78-7 as a white solid 30 mg (7%). ¹H NMR (400 MHz, DMSO-d6) δ 8.38-8.20 (m, 1H), 8.05 (bs, 2H), 7.41 and 7.28 (2s, 1H), 5.35-4.95 (m, 1H), 4.78 and 4.75 (2s, 2H), 4.64 and 4.61 (2s, 2H), 4.20-3.85 (m, 3H), 3.56-3.10 (m, 2H), 2.90-2.60 (m, 1H), 2.5-2.30 (m, 1H), 2.12 and 2.10 (2s, 3H), 1.43 and 1.37 (2d, 3H), 1.21 and 1.01 (2t, 3H); m/z [M+H]⁺ 540.3.

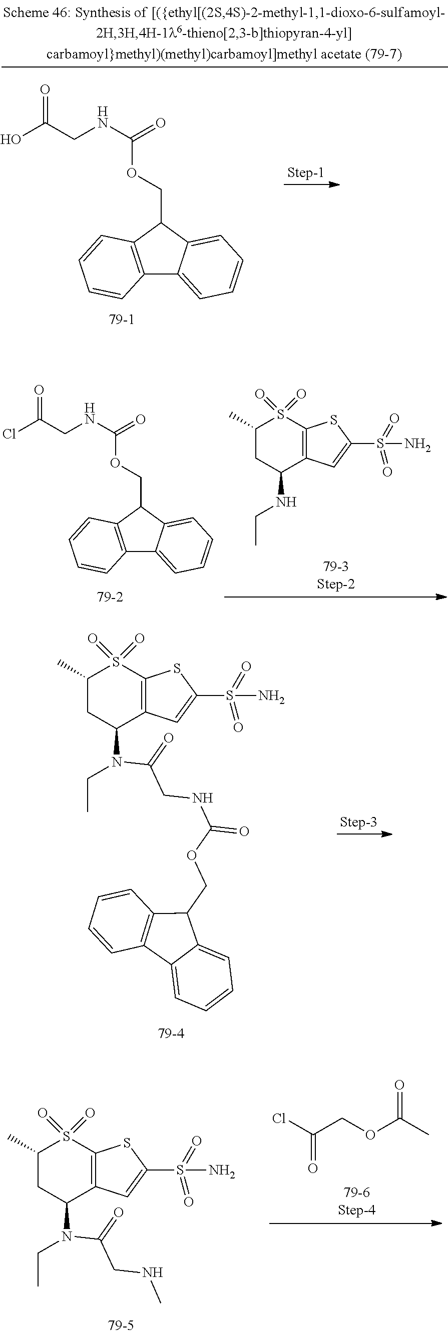

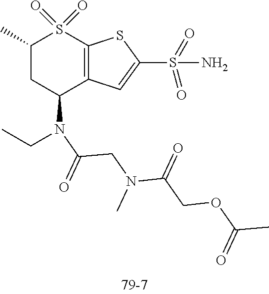 [SMILES: CCN(C(=O)CN(C)C(=O)COC(C)=O)[C@H]1C[C@H](C)S(=O)(=O)c2sc(S(N)(=O)=O)cc21]

Step-1 & 2: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate (79-4): To a solution of ({[(9H-fluoren-9-yl)methoxy]carbonyl}amino)acetic acid 79-1 (1.2 g, 4.1 mmol) in dichloromethane (20 mL), were added oxalyl chloride (0.7 mL, 8.2 mmol) and N,N-dimethylformamide (0.1 mL) at 0° C. The reaction mixture was allowed to stir at room temperature over a period of 30 min and concentrated the reaction mixture to dryness under nitrogen atmosphere. The residue was diluted with dichloromethane (100 mL) and added N,N-diisopropylethylamine (0.97 mL, 5.5 mmol) followed by dorzolamide 79-3 (1.0 g, 2.7 mmol) at 0° C. The reaction mixture was allowed to stir at room temperature over a period of 1 h. The resulting reaction mass was quenched with water (50 mL), extracted with dichloromethane (2×100 mL), organic layer was dried over sodium sulphate and concentrated under reduced pressure. The crude product obtained upon evaporation of volatiles was purified by silica gel column chromatography to obtain product 79-4 as a white solid 0.56 g (35%).

Step-3: Preparation of [({ethyl[(2S,4S)-2-methyl-1,1-dioxo-6-sulfamoyl-2H,3H,4H-1λ⁶-thieno[2,3-b]thiopyran-4-yl]carbamoyl}methyl)(methyl)carbamoyl]methyl acetate (79-5): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate 79-4 (0.43 g, 0.0074 mmol) in dichloromethane (5 V) was added piperidine (0.39 mL, 3.7 mmol) at 0° C. The reaction mixture was allowed to stir at 25-30° C. over a period of 24 hours. The resulting reaction mixture concentrated under reduced pressure to obtain compound 79-5 as an off white solid 0.25 g (40%). The crude compound was taken forward to next step without any purification.

Step-4: Preparation of (2S)-1-[(1S)-1-[({ethyl[(2S,4S)-2-methyl-1,1-dioxo-6-sulfamoyl-2H,3H,4H-1λ⁶-thieno[2,3-b]thiopyran-4-yl]carbamoyl}methyl)(methyl)carbamoyl]ethoxy]-1-oxopropan-2-yl (2S)-2-(acetyloxy)propanoate (79-7): To a solution of 2-amino-N-ethyl-N-[(4S,6S)-6-methyl-7,7-dioxo-2-sulfamoyl-4,5,6,7-tetrahydro-7l6-thieno[2,3-b]thiopyran-4-yl]acetamide 79-5 (0.3 g, 0.75 mmol) in dichloromethane (10 mL) were added N,N-Diisopropylethylamine (0.2 mL, 1.13 mmol) and acetoxyacetyl chloride 79-6 (0.072 mL, 0.68 mmol) at 0° C. The reaction mixture was allowed to stir at 25-30° C. over a period of 16 h. The resulting reaction mass was quenched with water (50 mL), extracted with ethyl acetate (100×2 mL), dried over sodium sulphate and concentrated under reduced pressure. The crude compound was purified by preparative HPLC to obtain product 79-7 as a white solid 0.11 g (29%). ¹H NMR (400 MHz, DMSO-d6) δ 8.04 (bs, 2H), 7.44, 7.41, 7.30 and 7.26 (4s, 1H), 5.32-4.95 (m, 1H), 4.87-4.09 (m, 4H), 3.99-3.85 (m, 1H), 3.51-3.04 (m, 2H), 2.99 and 2.91 (2s, 3H), 2.85-2.55 (m, 1H), 2.5-2.31 (m, 1H), 2.07 and 2.05 (2s, 3H), 1.49-1.33 (m, 3H), 1.26-0.96 (m, 3H); m/z [M+H]⁺ 496.3.

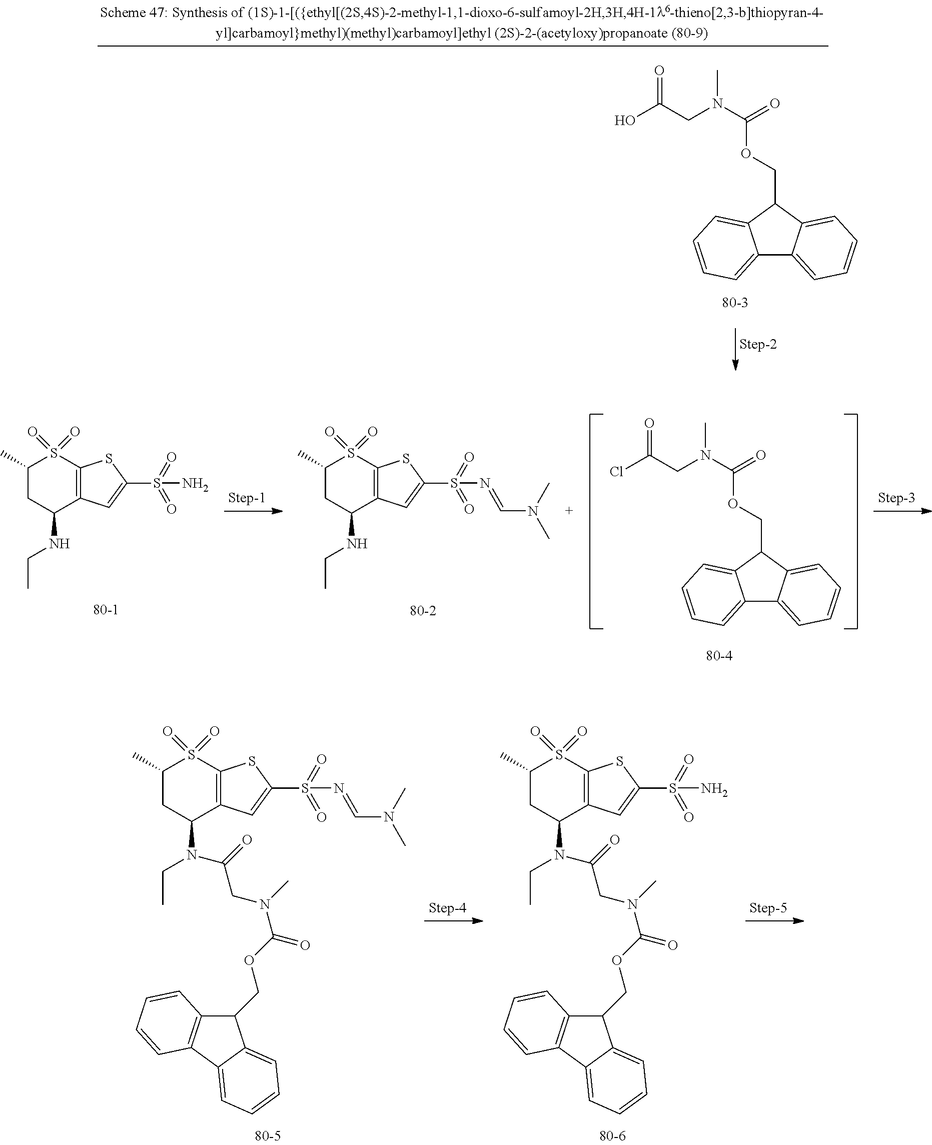

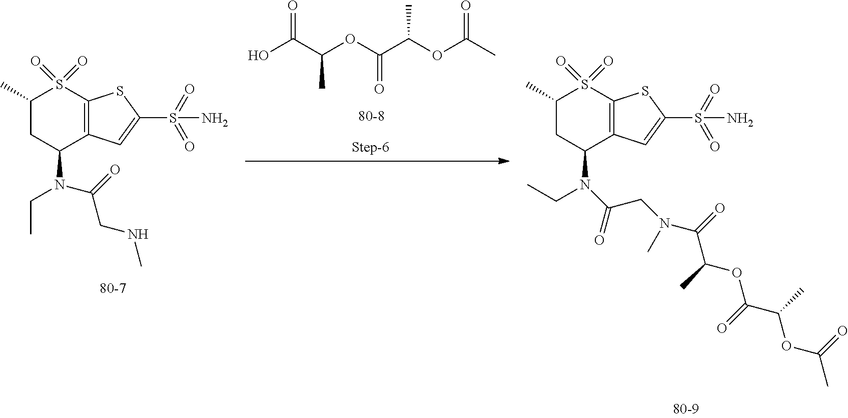

Step-1: Preparation of (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide (80-2): To a solution of (2S,4S)-4-(ethylamino)-2-methyl-1,1-dioxo-2H,3H,4H-1λ⁶-thieno[2,3-b]thiopyran-6-sulfonamide 80-1 (0.3 g, 0.83 mmol) in N,N-Dimethylformamide (0.6 mL), were added trimethylamine (0.12 mL, 0.91 mmol) and N,N-dimethylformamide dimethylacetal (0.13 mL, 0.99 mmol) at 0° C. The reaction mixture was allowed to stir at room temperature over a period of 16 hr. The resulting reaction mass was quenched with water (80 mL), extracted with dichloromethane (2×100 mL), organic layer was dried over sodium sulphate and concentrated under reduced pressure. The crude product obtained upon evaporation of volatiles was purified by silica gel column chromatography to obtain product 80-4 as a white solid 0.3 g (95%).

Step-2 & 3: Preparation of 9H-fluoren-9-ylmethyl N-({[(2S,4S)-6-{[(E)-[(dimethylamino)methylidene]amino]sulfonyl}-2-methyl-1,1-dioxo-2H,3H,4H-1λ⁶-thieno[2,3-b]thiopyran-4-yl](ethyl)carbamoyl}methyl)-N-methylcarbamate (80-5): To a solution of 2-{[(9H-fluoren-9-ylmethoxy)carbonyl](methyl)amino}acetic acid 80-3 (0.36 g, 1.18 mmol) in dichloromethane (20 mL), were added oxalyl chloride (0.29 mL, 3.4 mmol) and N,N-dimethylformamide (0.1 mL) at 0° C. The reaction mixture was allowed to stir at room temperature over a period of 30 min and concentrated to dryness under nitrogen atmosphere. The residue was diluted with dichloromethane (50 mL) and added N,N-diisopropylethylamine (0.28 mL, 1.5 mmol) followed by (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide 80-2 (0.3 g, 0.79 mmol) at 0° C. The reaction mixture was allowed to stir at room temperature over a period of 1 h. The resulting reaction mass was quenched with water (50 mL), extracted with dichloromethane (2×100 mL), organic layer was dried over sodium sulphate and concentrated under reduced pressure to obtain product 80-5 as colorless wax 0.45 g (87%). The crude compound as such taken into next step without any purification.

Step-4: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate (80-6): To a solution of 9H-fluoren-9-ylmethyl N-({[(2S,4S)-6-{[(E)-[(dimethylamino)methylidene]amino]sulfonyl}-2-methyl-1,1-dioxo-2H,3H,4H-1λ⁶-thieno[2,3-b]thiopyran-4-yl](ethyl)carbamoyl}methyl)-N-methylcarbamate 80-5 (2.5 g, 3.72 mmol) in methanol (10 mL) was added 50% aqueous HCl solution at room temperature. The reaction mixture was allowed to stir at 50° C. over a period of 12 h. Further the reaction mixture was allowed to stir at 100° C. over a period of 12 h. The resulting reaction mass was quenched with water (50 mL), extracted with ethyl acetate (100×2 mL), dried over sodium sulphate and concentrated under reduced pressure to obtain product 80-6 as an off white solid 2.1 g (95%).

Step-5: Preparation of 2-amino-N-ethyl-N-[(2S,4S)-2-methyl-1,1-dioxo-6-sulfamoyl-2H,3H,4H-1λ⁶-thieno[2,3-b]thiopyran-4-yl]acetamide (80-7): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate 80-6 (1.8 g, 2.91 mmol) in dichloromethane (5 V) was added piperidine (1.44 mL, 14.5 mmol) at 0° C. The reaction mixture was allowed to stir at 25-30° C. over a period of 4 hours. The resulting reaction mixture was concentrated under reduced pressure to obtain product 80-7 as an off white solid 1.0 g (86%).

Step-6: Preparation of (1S)-1-[({ethyl[(2S,4S)-2-methyl-1,1-dioxo-6-sulfamoyl-2H,3H,4H-1λ⁶-thieno[2,3-b]thiopyran-4-yl]carbamoyl}methyl)(methyl)carbamoyl]ethyl (2S)-2-(acetyloxy)propanoate (80-9): To a solution of (2S)-2-{[(2S)-2-(acetyloxy)propanoyl]oxy}propanoic acid 80-8 (0.15 g, 0.75 mmol) in dichloromethane (20 mL), were added oxalyl chloride (0.12 mL, 1.47 mmol) and N,N-dimethylformamide (0.1 mL) at 0° C. The reaction mixture was allowed to stir at room temperature over a period of 30 min and concentrated to dryness under nitrogen atmosphere. The residue was diluted with dichloromethane (80 mL) and added N,N-diisopropylethylamine (0.18 mL, 1.01 mmol) followed by 2-amino-N-ethyl-N-[(2S,4S)-2-methyl-1,1-dioxo-6-sulfamoyl-2H,3H,4H-1λ⁶-thieno[2,3-b]thiopyran-4-yl]acetamide 80-7 (0.2 g, 0.5 mmol) at 0° C. The reaction mixture was allowed to stir at room temperature over a period of 16 h. The resulting reaction mass was quenched with water (50 mL), extracted with dichloromethane (2×100 mL), organic layer was dried over sodium sulphate and concentrated under reduced pressure. The crude compound was purified by preparative HPLC to obtain product 80-9 as a white solid 0.1 g (34%). ¹H NMR (400 MHz, DMSO-d6/TFA) δ 8.08 and 8.02 (2bs, 2H), 7.44, 7.42, 7.28 and 7.26 (4s, 1H), 5.31-4.85 (m, 3H), 4.68, 4.38, 4.19 and 4.09 (4d, 2H), 3.95-3.82 (m, 1H), 3.55-2.55 (m, 6H), 2.5-2.32 (m, 1H), 2.05 and 2.04 (2s, 3H), 1.45-0.95 (m, 12H); m/z [M+H]⁺ 582.4.

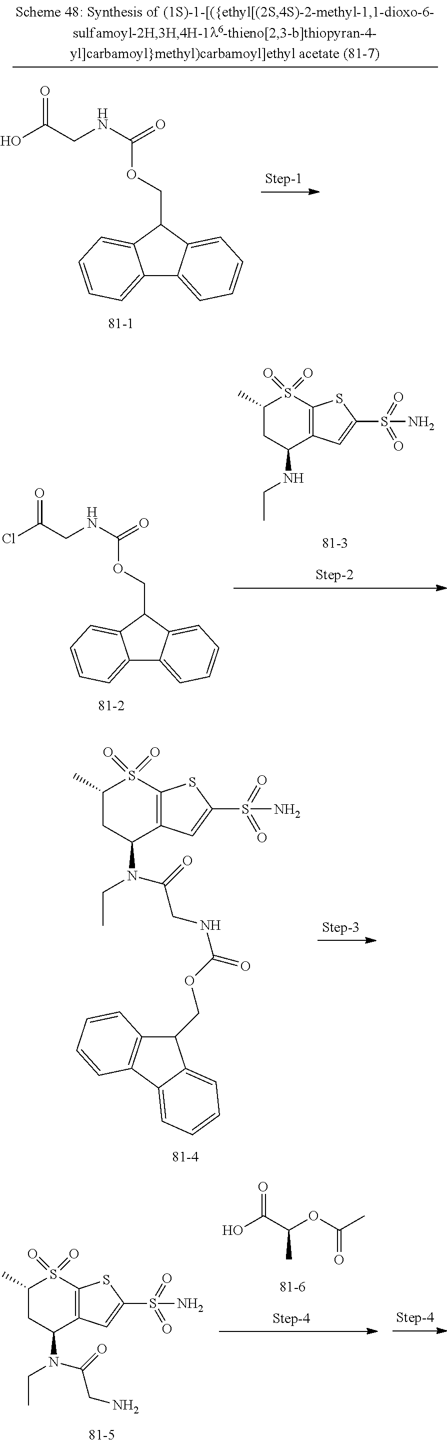

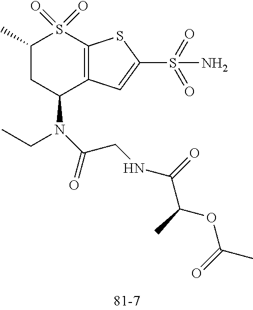 [SMILES: CCN(C(=O)CNC(=O)[C@H](C)OC(C)=O)[C@H]1C[C@H](C)S(=O)(=O)c2sc(S(N)(=O)=O)cc21]

Step-1 & 2: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate (81-4): To a solution of ({[(9H-fluoren-9-yl)methoxy]carbonyl}amino)acetic acid 81-1 (1.2 g, 4.1 mmol) in dichloromethane (20 mL), were added oxalyl chloride (0.7 mL, 8.2 mmol) and N,N-dimethylformamide (0.1 mL) at 0° C. The reaction mixture was allowed to stir at room temperature over a period of 30 min and concentrated the reaction mixture to dryness under nitrogen atmosphere. The residue was diluted with dichloromethane (100 mL) and added N,N-diisopropylethylamine (0.97 mL, 5.5 mmol) followed by dorzolamide 81-3 (1.0 g, 2.7 mmol) at 0° C. The reaction mixture was allowed to stir at room temperature over a period of 1 h. The resulting reaction mass was quenched with water (50 mL), extracted with dichloromethane (2×100 mL), organic layer was dried over sodium sulphate and concentrated under reduced pressure. The crude product obtained upon evaporation of volatiles was purified by silica gel column chromatography to obtain product 81-4 as a white solid 0.56 g (35%).

Step-3: Preparation of 2-amino-N-ethyl-N-[(4S,6S)-6-methyl-7,7-dioxo-2-sulfamoyl-4,5,6,7-tetrahydro-7l6-thieno[2,3-b]thiopyran-4-yl]acetamide (81-5): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate 81-4 (0.43 g, 0.0074 mmol) in dichloromethane (5 V) was added piperidine (0.39 mL, 3.7 mmol) at 0° C. The reaction mixture was allowed to stir at 25-30° C. over a period of 24 hours. The resulting reaction mixture was concentrated under reduced pressure to obtain compound 81-5 as an off white solid 0.25 g (40%). The crude compound was taken forward to next step without any purification.

Step-4: Preparation of (1S)-1-[({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamoyl]ethyl acetate (81-7): To a solution of (2S)-2-(acetyloxy)propanoic acid 81-6 (0.25 g, 1.96 mmol) in dichloromethane (10 mL) were added EDC.HCl (0.45 g, 2.3 mmol), N,N-Diisopropylethylamine (0.47 mL, 2.61 mmol) and 2-amino-N-ethyl-N-[(2S,4S)-2-methyl-1,1-dioxo-6-sulfamoyl-2H,3H,4H-1λ⁶-thieno[2,3-b]thiopyran-4-yl]acetamide 81-5 (0.5 g, 1.31 mmol) at 0° C. The reaction mixture was allowed to stir at 25-30° C. over a period of 2 h. The resulting reaction mass was quenched with water (50 mL), extracted with ethyl acetate (100 mL×2), dried over sodium sulphate and concentrated under reduced pressure. The crude compound was purified by reverse phase column chromatography to obtain product 81-7 as a white solid 0.18 g (27%). %). ¹H NMR (400 MHz, DMSO-d6/TFA) δ 8.22 and 8.16 (2t, 1H), 8.09 and 8.03 (2bs, 2H), 7.41, and 7.27 (2s, 1H), 5.32-4.95 (m, 2H), 4.39-3.82 (m, 3H), 3.51-3.09 (m, 2H), 2.86-2.30 (m, 2H), 2.06 and 2.04 (2s, 3H), 1.51-1.23- (m, 6H), 1.19 and 1.01 (2t, 3H),; m/z [M+H]⁺ 494.2.

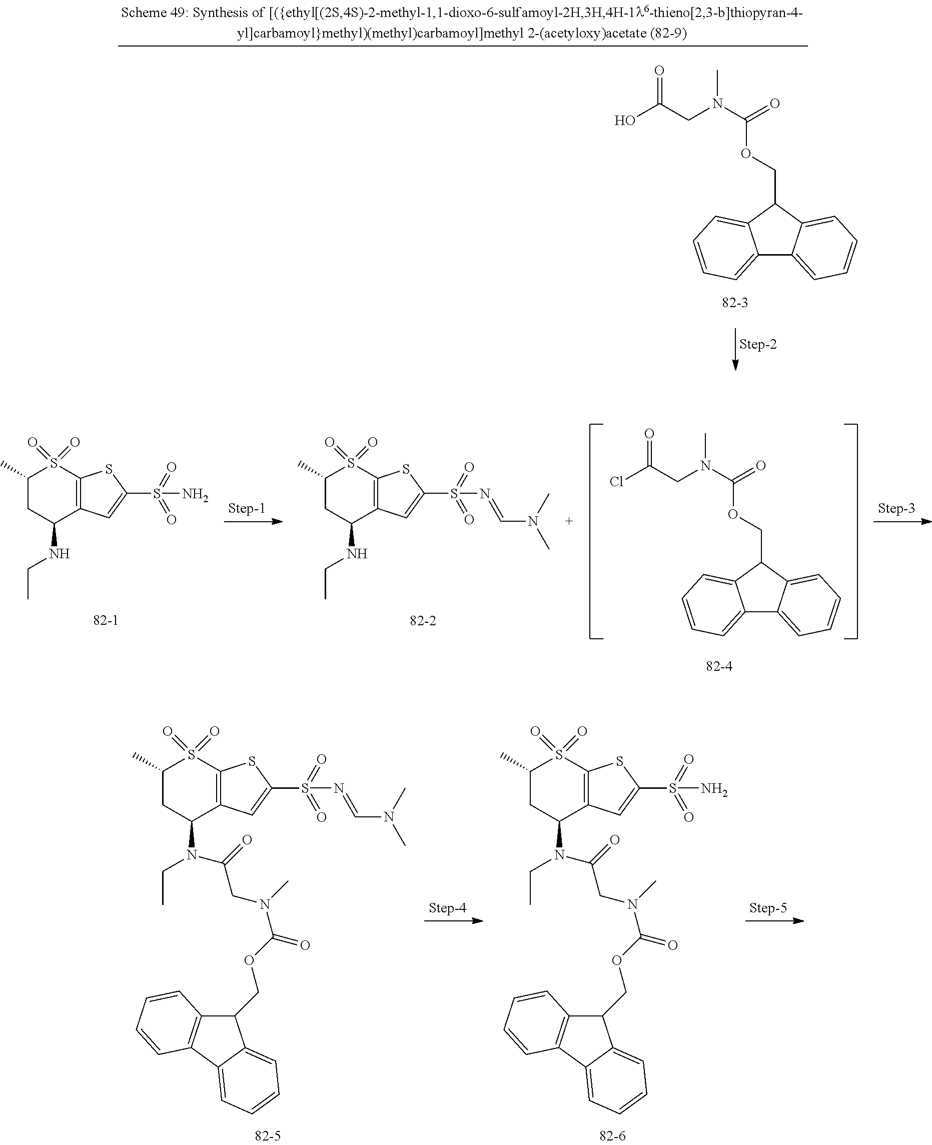

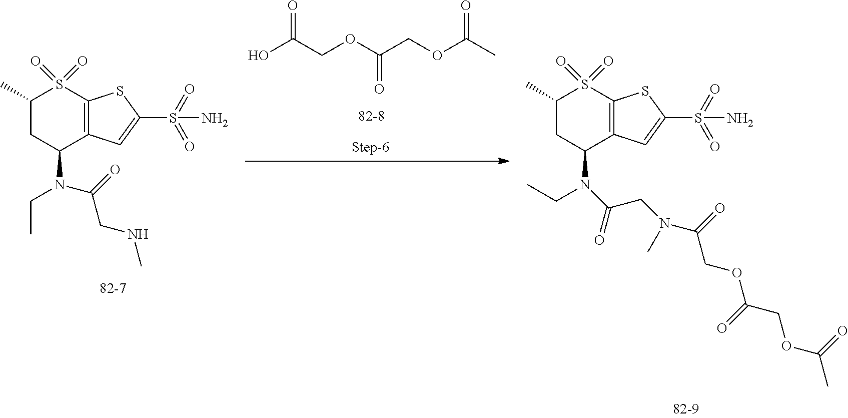

Step-1: Preparation of (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide (82-2): To a solution of (2S,4S)-4-(ethylamino)-2-methyl-1,1-dioxo-2H,3H,4H-1λ⁶-thieno[2,3-b]thiopyran-6-sulfonamide 82-1 (0.3 g, 0.83 mmol) in N,N-Dimethylformamide (0.6 mL), were added trimethylamine (0.12 mL, 0.91 mmol) and N,N-dimethylformamide dimethylacetal (0.13 mL, 0.99 mmol) at 0° C. The reaction mixture was allowed to stir at room temperature over a period of 16 hr. The resulting reaction mass was quenched with water (80 mL), extracted with dichloromethane (2×100 mL), organic layer was dried over sodium sulphate and concentrated under reduced pressure. The crude product obtained upon evaporation of volatiles was purified by silica gel column chromatography to obtain product 82-4 as a white solid 0.3 g (95%).

Step-2 & 3: Preparation of 9H-fluoren-9-ylmethyl N-({[(2S,4S)-6-{[(E)-[(dimethylamino)methylidene]amino]sulfonyl}-2-methyl-1,1-dioxo-2H,3H,4H-1λ⁶-thieno[2,3-b]thiopyran-4-yl](ethyl)carbamoyl}methyl)-N-methylcarbamate (82-5): To a solution of 2-{[(9H-fluoren-9-ylmethoxy)carbonyl](methyl)amino}acetic acid 82-3 (0.36 g, 1.18 mmol) in dichloromethane (20 mL), were added oxalyl chloride (0.29 mL, 3.4 mmol) and N,N-dimethylformamide (0.1 mL) at 0° C. The reaction mixture was allowed to stir at room temperature over a period of 30 min and concentrated to dryness under nitrogen atmosphere. The residue was diluted with dichloromethane (50 mL) and added N,N-diisopropylethylamine (0.28 mL, 1.5 mmol) followed by (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide 82-2 (0.3 g, 0.79 mmol) at 0° C. The reaction mixture was allowed to stir at room temperature over a period of 1 h. The resulting reaction mass was quenched with water (50 mL), extracted with dichloromethane (2×100 mL), organic layer was dried over sodium sulphate and concentrated under reduced pressure to obtain product 82-5 as colorless wax 0.45 g (87%). The crude compound as such taken into next step without any purification.

Step-4: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate (82-6): To a solution of 9H-fluoren-9-ylmethyl N-({[(2S,4S)-6-{[(E)-[(dimethylamino)methylidene]amino]sulfonyl}-2-methyl-1,1-dioxo-2H,3H,4H-1λ⁶-thieno[2,3-b]thiopyran-4-yl](ethyl)carbamoyl}methyl)-N-methylcarbamate 82-5 (2.5 g, 3.72 mmol) in methanol (10 mL) was added 50% aqueous HCl solution at room temperature. The reaction mixture was allowed to stir at 50° C. over a period of 12 h. Further the reaction mixture was allowed to stir at 100° C. over a period of 12 h. The resulting reaction mass was quenched with water (50 mL), extracted with ethyl acetate (100×2 mL), dried over sodium sulphate and concentrated under reduced pressure to obtain product 82-6 as an off white solid 2.1 g (95%).

Step-5: Preparation of 2-amino-N-ethyl-N-[(2S,4S)-2-methyl-1,1-dioxo-6-sulfamoyl-2H,3H,4H-1λ⁶-thieno[2,3-b]thiopyran-4-yl-]acetamide (82-7): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate 82-6 (1.8 g, 2.91 mmol) in dichloromethane (5 V) was added piperidine (1.44 mL, 14.5 mmol) at 0° C. The reaction mixture was allowed to stir at 25-30° C. over a period of 4 hours. The resulting reaction mixture was concentrated under reduced pressure to obtain product 82-7 as an off white solid 1.0 g (86%).

Step-6: Preparation of [({ethyl[(2S,4S)-2-methyl-1,1-dioxo-6-sulfamoyl-2H,3H,4H-1λ⁶-thieno[2,3-b]thiopyran-4-yl]carbamoyl}methyl)(methyl)carbamoyl]methyl 2-(acetyloxy)acetate (82-9): To a solution of {[(acetyloxy)acetyl]oxy}acetic acid 82-8 (0.33 g, 1.89 mmol) in dichloromethane (20 mL), were added oxalyl chloride (0.31 mL, 3.79 mmol) and N,N-dimethylformamide (0.1 mL) at 0° C. The reaction mixture was allowed to stir at room temperature over a period of 30 min and concentrated to dryness under nitrogen atmosphere. The residue was diluted with dichloromethane (100 mL) and added N,N-diisopropylethylamine (0.45 mL, 2.53 mmol) followed by 2-amino-N-ethyl-N-[(2S,4S)-2-methyl-1,1-dioxo-6-sulfamoyl-2H,3H,4H-1λ⁶-thieno[2,3-b]thiopyran-4-yl]acetamide 82-7 (0.5 g, 1.26 mmol) at 0° C. The reaction mixture was allowed to stir at room temperature over a period of 16 h. The resulting reaction mass was quenched with water (50 mL), extracted with dichloromethane (2×100 mL), organic layer was dried over sodium sulphate and concentrated under reduced pressure. The crude compound was purified by preparative HPLC to obtain product 82-9 as a white solid 0.17 g (24%). ¹H NMR (400 MHz, DMSO-d6) δ 8.03 (bs, 2H), 7.48, 7.42, 7.31 and 7.27 (4s, 1H), 5.32-4.09 (m, 7H), 4.01-3.85 (m, 1H), 3.51-3.05 (m, 2H), 3.00-2.55 (m, 4H), 2.5-2.33 (m, 1H), 2.10 (s, 3H), 1.47-1.34 (m, 3H), 1.27-0.96 (m, 3H); m/z [M+H]⁺ 554.3.

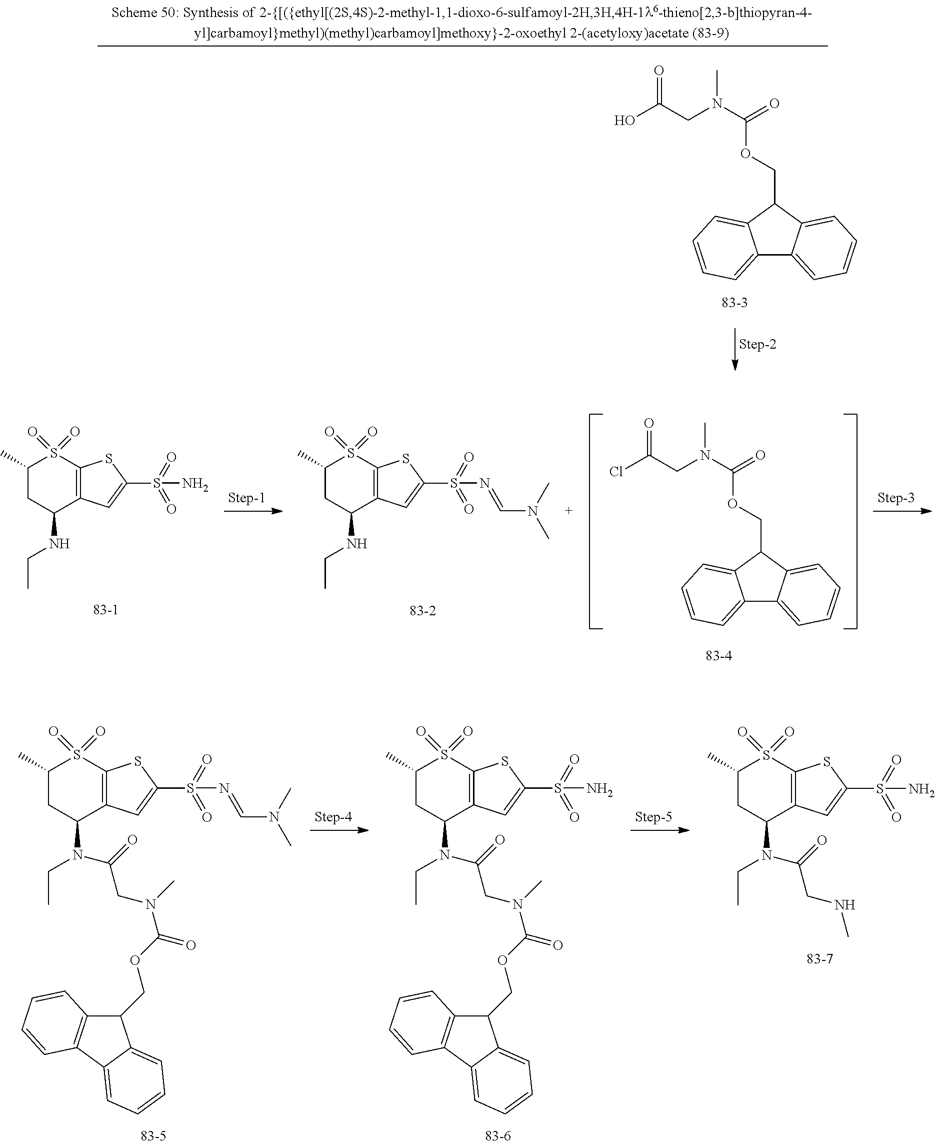

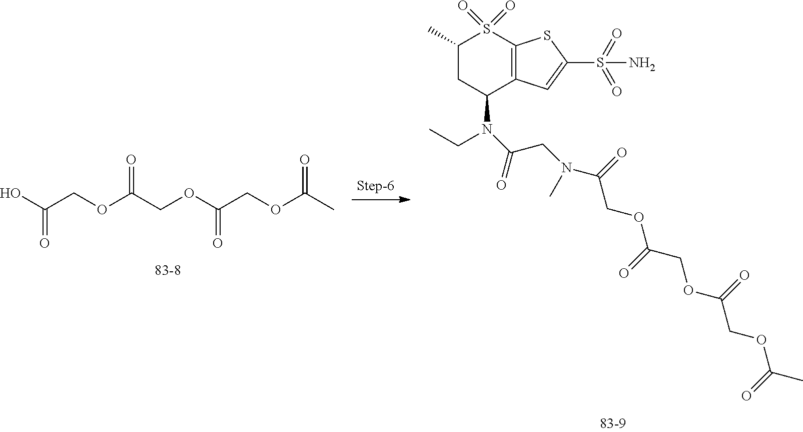 [SMILES: CC(=O)OCC(=O)OCC(=O)OCC(=O)O ; CCN(C(=O)CN(C)C(=O)COC(=O)COC(=O)COC(C)=O)[C@H]1C[C@H](C)S(=O)(=O)c2sc(S(N)(=O)=O)cc21]

Step-1: Preparation of (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide (83-2): To a solution of (2S,4S)-4-(ethylamino)-2-methyl-1,1-dioxo-2H,3H,4H-1λ⁶-thieno[2,3-b]thiopyran-6-sulfonamide 83-1 (0.3 g, 0.83 mmol) in N,N-Dimethylformamide (0.6 mL), were added trimethylamine (0.12 mL, 0.91 mmol) and N,N-dimethylformamide dimethylacetal (0.13 mL, 0.99 mmol) at 0° C. The reaction mixture was allowed to stir at room temperature over a period of 16 hr. The resulting reaction mass was quenched with water (80 mL), extracted with dichloromethane (2×100 mL), organic layer was dried over sodium sulphate and concentrated under reduced pressure. The crude product obtained upon evaporation of volatiles was purified by silica gel column chromatography to obtain product 83-4 as a white solid 0.3 g (95%).

Step-2 & 3: Preparation of 9H-fluoren-9-ylmethyl N-({[(2S,4S)-6-{[(E)-[(dimethylamino)methylidene]amino]sulfonyl}-2-methyl-1,1-dioxo-2H,3H,4H-1λ⁶-thieno[2,3-b]thiopyran-4-yl](ethyl)carbamoyl}methyl)-N-methylcarbamate (83-5): To a solution of 2-{[(9H-fluoren-9-ylmethoxy)carbonyl](methyl)amino}acetic acid 83-3 (0.36 g, 1.18 mmol) in dichloromethane (20 mL), were added oxalyl chloride (0.29 mL, 3.4 mmol) and N,N-dimethylformamide (0.1 mL) at 0° C. The reaction mixture was allowed to stir at room temperature over a period of 30 min and concentrated to dryness under nitrogen atmosphere. The residue was diluted with dichloromethane (50 mL) and added N,N-diisopropylethylamine (0.28 mL, 1.5 mmol) followed by (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide 83-2 (0.3 g, 0.79 mmol) at 0° C. The reaction mixture was allowed to stir at room temperature over a period of 1 h. The resulting reaction mass was quenched with water (50 mL), extracted with dichloromethane (2×100 mL), organic layer was dried over sodium sulphate and concentrated under reduced pressure to obtain product 83-5 as colorless wax 0.45 g (87%). The crude compound as such taken into next step without any purification.

Step-4: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate (83-6): To a solution of 9H-fluoren-9-ylmethyl N-({[(2S,4S)-6-{[(E)-[(dimethylamino)methylidene]amino]sulfonyl}-2-methyl- 1,1-dioxo-2H,3H,4H-1λ⁶-thieno[2,3-b]thiopyran-4-yl](ethyl)carbamoyl}methyl)-N-methylcarbamate 83-5 (2.5 g, 3.72 mmol) in methanol (10 mL) was added 50% aqueous HCl solution at room temperature. The reaction mixture was allowed to stir at 50° C. over a period of 12 h. Further the reaction mixture was allowed to stir at 100° C. over a period of 12 h. The resulting reaction mass was quenched with water (50 mL), extracted with ethyl acetate (100×2 mL), dried over sodium sulphate and concentrated under reduced pressure to obtain product 83-6 as an off white solid 2.1 g (95%).

Step-5: Preparation of 2-amino-N-ethyl-N-[(2S,4S)-2-methyl-1,1-dioxo-6-sulfamoyl-2H,3H,4H-1λ⁶-thieno[2,3-b]thiopyran-4-yl]acetamide (83-7): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate 83-6 (1.8 g, 2.91 mmol) in dichloromethane (5 V) was added piperidine (1.44 mL, 14.5 mmol) at 0° C. The reaction mixture was allowed to stir at 25-30° C. over a period of 4 hours. The resulting reaction mixture was concentrated under reduced pressure to obtain product 83-7 as an off white solid 1.0 g (86%).

Step-6: Preparation of [({ethyl[(2S,4S)-2-methyl-1,1-dioxo-6-sulfamoyl-2H,3H,4H-1λ⁶-thieno[2,3-b]thiopyran-4-yl]carbamoyl}methyl)(methyl)carbamoyl]methyl 2-(acetyloxy)acetate (83-9): To a solution of {[(acetyloxy)acetyl]oxy}acetic acid 83-8 (0.33 g, 1.89 mmol) in dichloromethane (20 mL), were added oxalyl chloride (0.31 mL, 3.79 mmol) and N,N-dimethylformamide (0.1 mL) at 0° C. The reaction mixture was allowed to stir at room temperature over a period of 30 min and concentrated to dryness under nitrogen atmosphere. The residue was diluted with dichloromethane (100 mL) and added N,N-diisopropylethylamine (0.45 mL, 2.53 mmol) followed by 2-amino-N-ethyl-N-[(2S,4S)-2-methyl-1,1-dioxo-6-sulfamoyl-2H,3H,4H-1λ⁶-thieno[2,3-b]thiopyran-4-yl]acetamide 83-7 (0.5 g, 1.26 mmol) at 0° C. The reaction mixture was allowed to stir at room temperature over a period of 16 h. The resulting reaction mass was quenched with water (50 mL), extracted with dichloromethane (2×100 mL), organic layer was dried over sodium sulphate and concentrated under reduced pressure. The crude compound was purified by preparative HPLC to obtain product 9 as a white solid 0.17 g (24%). ¹H NMR (400 MHz, DMSO-d6) δ 8.11 and 8.02 (2bs, 2H), 7.48, 7.43, 7.31 and 7.27 (4s, 1H), 5.31-4.09 (m, 9H), 4.00-3.85 (m, 1H), 3.51-3.05 (m, 2H), 3.01-2.55 (m, 4H), 2.5-2.33 (m, 1H), 2.11 (s, 3H), 1.47-1.32 (m, 3H), 1.27-0.95 (m, 3H); m/z [M+H]⁺ 612.4.

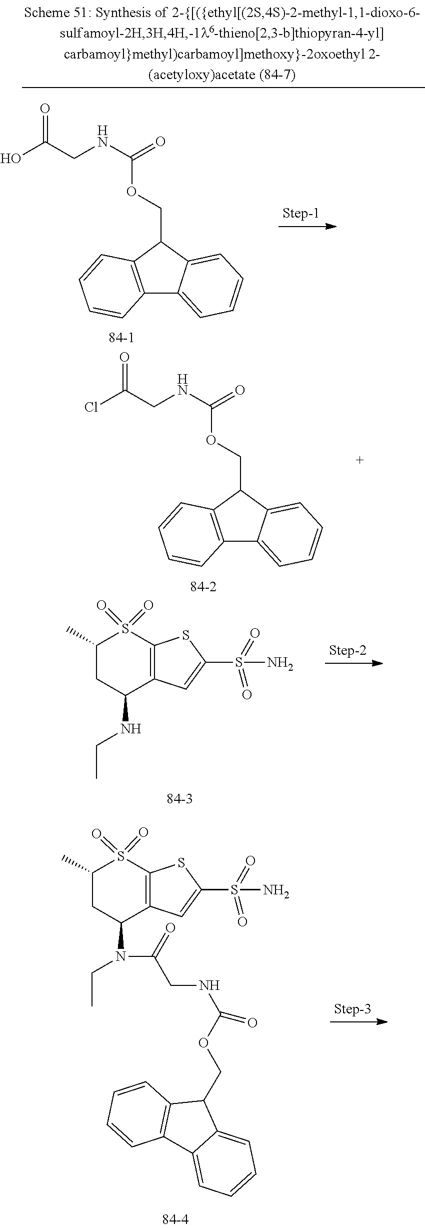

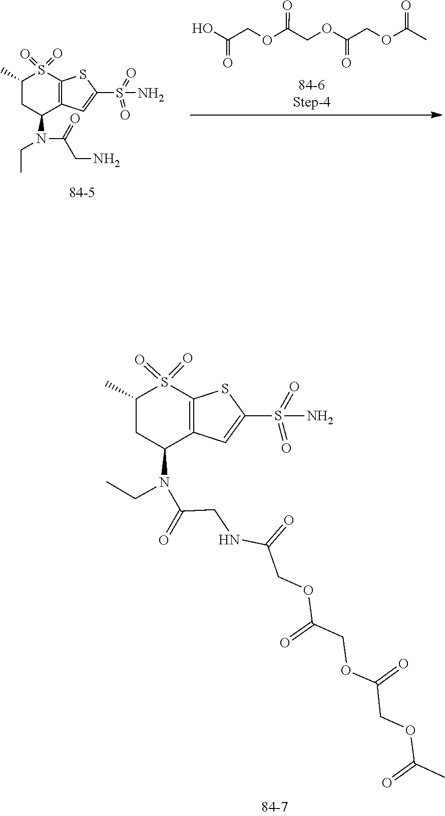 [SMILES: CCN(C(=O)CN)[C@H]1C[C@H](C)S(=O)(=O)c2sc(S(N)(=O)=O)cc21 ; CC(=O)OCC(=O)OCC(=O)OCC(=O)O ; CCN(C(=O)CNC(=O)COC(=O)COC(=O)COC(C)=O)[C@H]1C[C@H](C)S(=O)(=O)c2sc(S(N)(=O)=O)cc21]

Step-1 & 2: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate (84-4): To a solution of ({[(9H-fluoren-9-yl)methoxy]carbonyl}amino)acetic acid 84-1 (1.2 g, 4.1 mmol) in dichloromethane (20 mL), were added oxalyl chloride (0.7 mL, 8.2 mmol) and N,N-dimethylformamide (0.1 mL) at 0° C. The reaction mixture was allowed to stir at room temperature over a period of 30 min and concentrated the reaction mixture to dryness under nitrogen atmosphere. The residue was diluted with dichloromethane (100 mL) and added N,N-diisopropylethylamine (0.97 mL, 5.5 mmol) followed by dorzolamide 84-3 (1.0 g, 2.7 mmol) at 0° C. The reaction mixture was allowed to stir at room temperature over a period of 1 h. The resulting reaction mass was quenched with water (50 mL), extracted with dichloromethane (2×100 mL), organic layer was dried over sodium sulphate and concentrated under reduced pressure. The crude product obtained upon evaporation of volatiles was purified by silica gel column chromatography to obtain product 84-4 as a white solid 0.56 g (35%).

Step-3: Preparation of 2-amino-N-ethyl-N-[(4S,6S)-6-methyl-7,7-dioxo-2-sulfamoyl-4,5,6,7-tetrahydro-7l6-thieno[2,3-b]thiopyran-4-yl]acetamide (84-5): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate 84-4 (0.43 g, 0.0074 mmol) in dichloromethane (5 V) was added piperidine (0.39 mL, 3.7 mmol) at 0° C. The reaction mixture was allowed to stir at 25-30° C. over a period of 24 hours. The resulting reaction mixture was concentrated under reduced pressure to obtain compound 84-5 as an off white solid 0.25 g (40%). The crude compound was taken forward to next step without any purification.

Step-4: Preparation of 2-{[({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamoyl]methoxy}-2-oxoethyl 2-(acetyloxy)acetate (84-7): To a solution of [({[(acetyloxy)acetyl]oxy}acetyl)oxy]acetic acid 84-6 (0.460 g, 1.90 mmol) in dichloromethane (10 mL) were added EDC.HCl (0.451 g, 2.3 mmol), N,N-Diisopropylethylamine (0.47 mL, 2.61 mmol) and 2-amino-N-ethyl-N-[(4S,6S)-6-methyl-7,7-dioxo-2-sulfamoyl-4,5,6,7-tetrahydro-7□6-thieno[2,3-b]thiopyran-4-yl]acetamide 84-5 (0.5 g, 1.31 mmol) at 0° C. The reaction mixture was allowed to stir at 25-30° C. over a period of 2 h. The resulting reaction mass was quenched with water (50 mL), extracted with ethyl acetate (100×2 mL), dried over sodium sulphate and concentrated under reduced pressure. The crude compound was purified by reverse phase column chromatography to obtain product 84-7 as a white solid 38 mg (4%). ¹H NMR (400 MHz, DMSO-d6) δ 8.34 and 8.26 (2t, 1H), 8.09 and 8.04 (2bs, 2H), 7.41 and 7.28 (2s, 1H), 5.35-4.95 (m, 1H), 4.89 and 4.86 (2s, 2H), 4.78 and 4.77 (2s, 2H), 4.65 and 4.61 (2s, 2H), 4.42-3.86 (m, 3H), 3.55-3.10 (m, 2H), 2.89-2.60 (m, 1H), 2.5-2.30 (m, 1H), 2.10 (s, 3H), 1.43 and 1.37 (2d, 3H), 1.21 and 1.01 (2t, 3H); m/z [M+H]⁺ 598.4.

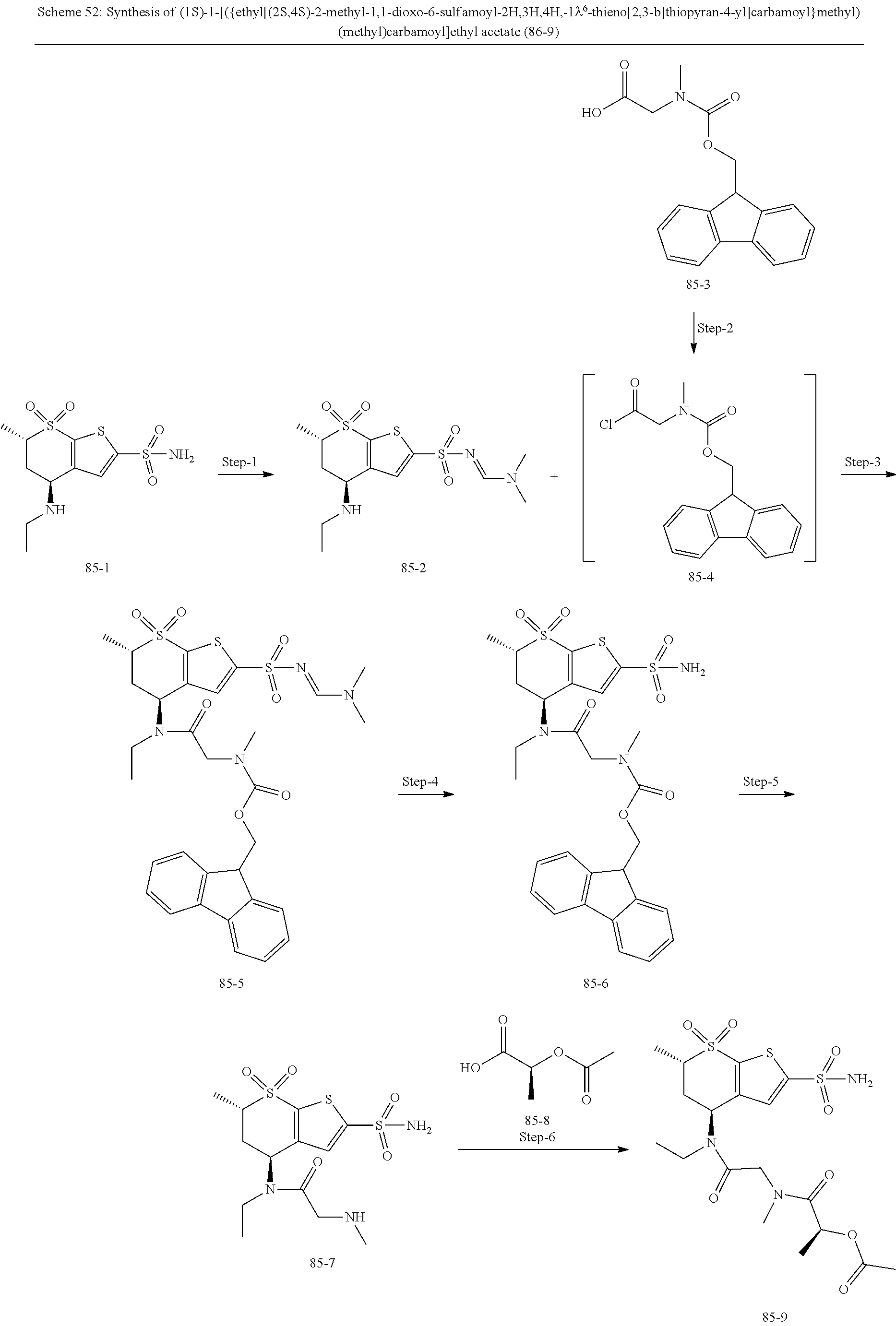

Step-1: Preparation of (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide (85-2): To a solution of (2S,4S)-4-(ethylamino)-2-methyl-1,1-dioxo-2H,3H,4H-1λ⁶-thieno[2,3-b]thiopyran-6-sulfonamide 85-1 (0.3 g, 0.83 mmol) in N,N-Dimethylformamide (0.6 mL), were added trimethylamine (0.12 mL, 0.91 mmol) and N,N-dimethylformamide dimethylacetal (0.13 mL, 0.99 mmol) at 0° C. The reaction mixture was allowed to stir at room temperature over a period of 16 hr. The resulting reaction mass was quenched with water (80 mL), extracted with dichloromethane (2×100 mL), organic layer was dried over sodium sulphate and concentrated under reduced pressure. The crude product obtained upon evaporation of volatiles was purified by silica gel column chromatography to obtain product 85-4 as a white solid 0.3 g (95%).

Step-2 & 3: Preparation of 9H-fluoren-9-ylmethyl N-({[(2S,45)-6-{[(E)-[(dimethylamino)methylidene]amino]sulfonyl}-2-methyl-1,1-dioxo-2H,3H,4H-1λ⁶-thieno[2,3-b]thiopyran-4-yl](ethyl)carbamoyl}methyl)-N-methylcarbamate (85-5): To a solution of 2-{[(9H-fluoren-9-ylmethoxy)carbonyl](methyl)amino}acetic acid 85-3 (0.36 g, 1.18 mmol) in dichloromethane (20 mL), were added oxalyl chloride (0.29 mL, 3.4 mmol) and N,N-dimethylformamide (0.1 mL) at 0° C. The reaction mixture was allowed to stir at room temperature over a period of 30 min and concentrated to dryness under nitrogen atmosphere. The residue was diluted with dichloromethane (50 mL) and added N,N-diisopropylethylamine (0.28 mL, 1.5 mmol) followed by (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide 85-2 (0.3 g, 0.79 mmol) at 0° C. The reaction mixture was allowed to stir at room temperature over a period of 1 h. The resulting reaction mass was quenched with water (50 mL), extracted with dichloromethane (2×100 mL), organic layer was dried over sodium sulphate and concentrated under reduced pressure to obtain product 85-5 as colorless wax 0.45 g (87%). The crude compound as such taken into next step without any purification.

Step-4: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate (85-6): To a solution of 9H-fluoren-9-ylmethyl N-({[(2S,4S)-6-{[(E)-[(dimethylamino)methylidene]amino]sulfonyl}-2-methyl-1,1-dioxo-2H,3H,4H-1λ⁶-thieno[2,3-b]thiopyran-4-yl](ethyl)carbamoyl}methyl)-N-methylcarbamate 85-5 (2.5 g, 3.72 mmol) in methanol (10 mL) was added 50% aqueous HCl solution at room temperature. The reaction mixture was allowed to stir at 50° C. over a period of 12 h. Further the reaction mixture was allowed to stir at 100° C. over a period of 12 h. The resulting reaction mass was quenched with water (50 mL), extracted with ethyl acetate (100×2 mL), dried over sodium sulphate and concentrated under reduced pressure to obtain product 85-6 as an off white solid 2.1 g (95%).

Step-5: Preparation of 2-amino-N-ethyl-N-[(2S,4S)-2-methyl-1,1-dioxo-6-sulfamoyl-2H,3H,4H-1λ⁶-thieno[2,3-b]thiopyran-4-yl]acetamide (85-7): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate 85-6 (1.8 g, 2.91 mmol) in dichloromethane (5 V) was added piperidine (1.44 mL, 14.5 mmol) at 0° C. The reaction mixture was allowed to stir at 25-30° C. over a period of 4 hours. The resulting reaction mixture was concentrated under reduced pressure to obtain product 85-7 as an off white solid 1.0 g (86%).

Step-6: Preparation of (1S)-1-[({ethyl[(2S,4S)-2-methyl-1,1-dioxo-6-sulfamoyl-2H,3H,4H-1λ⁶-thieno[2,3-b]thiopyran-4-yl]carbamoyl}methyl)(methyl)carbamoyl]ethyl acetate (85-9): To a solution of (2S)-2-(acetyloxy)propanoic acid 85-8 (0.25 g, 1.89 mmol) in dichloromethane (10 mL) were added EDC.HCl (0.435 g, 2.27 mmol), N,N-Diisopropylethylamine (0.45 mL, 2.53 mmol) and 2-amino-N-ethyl-N-[(2S,4S)-2-methyl-1,1-dioxo-6-sulfamoyl-2H,3H,4H-1λ⁶-thieno[2,3-b]thiopyran-4-yl]acetamide 85-7 (0.5 g, 1.26 mmol) at 0° C. The reaction mixture was allowed to stir at 25-30° C. over a period of 2 h. The resulting reaction mass was quenched with water (50 mL), extracted with ethyl acetate (100×2 mL), dried over sodium sulphate and concentrated under reduced pressure. The crude compound was purified by reverse phase column chromatography to obtain product 85-9 as a white solid 0.2 g (31%). ¹H NMR (400 MHz, DMSO-d6) δ 8.03 (bs, 2H), 7.47, 7.38, 7.27 and 7.26 (4s, 1H), 5.44-4.95 (m, 2H), 4.89-3.85 (m, 3H), 3.55-3.10 (m, 2H), 3.06 and 3.01 (2s, 3H), 2.90-2.55 (m, 1H), 2.46-2.33 (m, 1H), 2.03 and 2.01 (2s, 3H), 1.48-0.96 (m, 9H); m/z [M+H]⁺ 510.4.

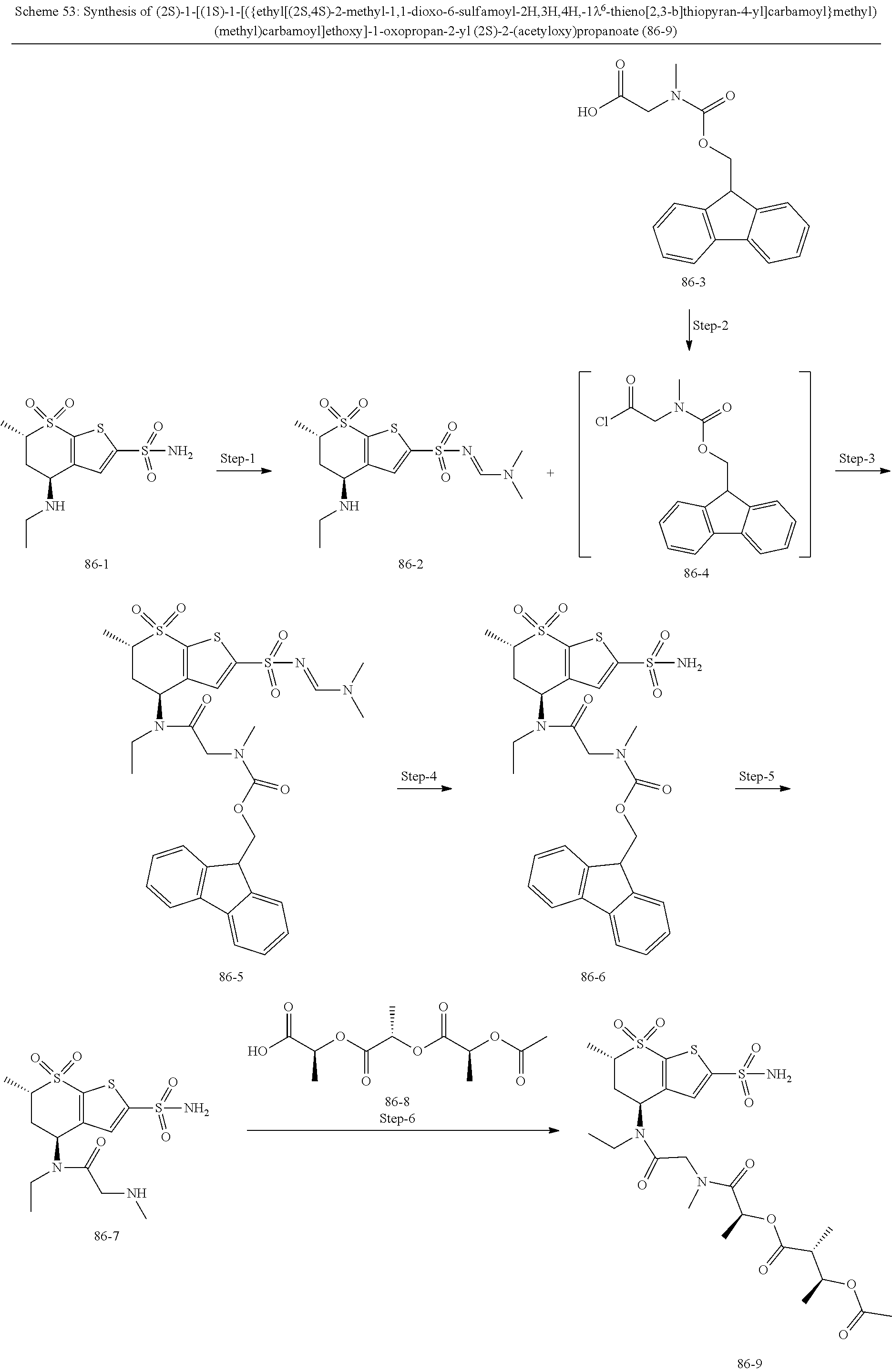

Step-1: Preparation of (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide (86-2): To a solution of (2S,4S)-4-(ethylamino)-2-methyl-1,1-dioxo-2H,3H,4H-1λ⁶-thieno[2,3-b]thiopyran-6-sulfonamide 86-1 (0.3 g, 0.83 mmol) in N,N-Dimethylformamide (0.6 mL), were added trimethylamine (0.12 mL, 0.91 mmol) and N,N-dimethylformamide dimethylacetal (0.13 mL, 0.99 mmol) at 0° C. The reaction mixture was allowed to stir at room temperature over a period of 16 hr. The resulting reaction mass was quenched with water (80 mL), extracted with dichloromethane (2×100 mL), organic layer was dried over sodium sulphate and concentrated under reduced pressure. The crude product obtained upon evaporation of volatiles was purified by silica gel column chromatography to obtain product 86-4 as a white solid 0.3 g (95%).

Step-2 & 3: Preparation of 9H-fluoren-9-ylmethyl N-({[(2S,4S)-6-{[(E)-[(dimethylamino)methylidene]amino]sulfonyl}-2-methyl-1,1-dioxo-2H,3H,4H-1λ⁶-thieno[2,3-b]thiopyran-4-yl](ethyl)carbamoyl}methyl)-N-methylcarbamate (86-5): To a solution of 2-{[(9H-fluoren-9-ylmethoxy)carbonyl](methyl)amino}acetic acid 86-3 (0.36 g, 1.18 mmol) in dichloromethane (20 mL), were added oxalyl chloride (0.29 mL, 3.4 mmol) and N,N-dimethylformamide (0.1 mL) at 0° C. The reaction mixture was allowed to stir at room temperature over a period of 30 min and concentrated to dryness under nitrogen atmosphere. The residue was diluted with dichloromethane (50 mL) and added N,N-diisopropylethylamine (0.28 mL, 1.5 mmol) followed by (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide 86-2 (0.3 g, 0.79 mmol) at 0° C. The reaction mixture was allowed to stir at room temperature over a period of 1 h. The resulting reaction mass was quenched with water (50 mL), extracted with dichloromethane (2×100 mL), organic layer was dried over sodium sulphate and concentrated under reduced pressure to obtain product 86-5 as colorless wax 0.45 g (87%). The crude compound as such taken into next step without any purification.

Step-4: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate (86-6): To a solution of 9H-fluoren-9-ylmethyl N-({[(2S,4S)-6-{[(E)-[(dimethylamino)methylidene]amino]sulfonyl}-2-methyl-1,1-dioxo-2H,3H,4H-1λ⁶-thieno[2,3-b]thiopyran-4-yl](ethyl)carbamoyl}methyl)-N-methylcarbamate 86-5 (2.5 g, 3.72 mmol) in methanol (10 mL) was added 50% aqueous HCl solution at room temperature. The reaction mixture was allowed to stir at 50° C. over a period of 12 h. Further the reaction mixture was allowed to stir at 100° C. over a period of 12 h. The resulting reaction mass was quenched with water (50 mL), extracted with ethyl acetate (100×2 mL), dried over sodium sulphate and concentrated under reduced pressure to obtain product 86-6 as an off white solid 2.1 g (95%).

Step-5: Preparation of 2-amino-N-ethyl-N-[(2S,4S)-2-methyl-1,1-dioxo-6-sulfamoyl-2H,3H,4H-1λ⁶-thieno[2,3-b]thiopyran-4-yl]acetamide (86-7): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate 86-6 (1.8 g, 2.91 mmol) in dichloromethane (5 V) was added piperidine (1.44 mL, 14.5 mmol) at 0° C. The reaction mixture was allowed to stir at 25-30° C. over a period of 4 hours. The resulting reaction mixture was concentrated under reduced pressure to obtain product 86-7 as an off white solid 1.0 g (86%).

Step-6: Preparation of (2S)-1-[(1S)-1-[({ethyl[(2S,4S)-2-methyl-1,1-dioxo-6-sulfamoyl-2H,3H,4H-1λ⁶-thieno[2,3-b]thiopyran-4-yl]carbamoyl}methyl)(methyl)carbamoyl]ethoxy]-1-oxopropan-2-yl (2S)-2-(acetyloxy)propanoate (86-9): To a solution of (2S)-2-{[(2S)-2-{[(2S)-2-(acetyloxy)propanoyl]oxy}propanoyl]oxy}propanoic acid 86-8 (0.52 g, 1.89 mmol) in dichloromethane (20 mL), were added oxalyl chloride (0.32 mL, 3.8 mmol) and N,N-dimethylformamide (0.1 mL) at 0° C. The reaction mixture was allowed to stir at room temperature over a period of 30 min and concentrated to dryness under nitrogen atmosphere. The residue was diluted with dichloromethane (100 mL) and added N,N-diisopropylethylamine (0.45 mL, 2.5 mmol) followed by 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate 86-7 (0.5 g, 1.26 mmol) at 0° C. The reaction mixture was allowed to stir at room temperature over a period of 16 h. The resulting reaction mass was quenched with water (50 mL), extracted with dichloromethane (2×100 mL), organic layer was dried over sodium sulphate and concentrated under reduced pressure. The crude compound was purified by preparative HPLC to obtain product 9 as a white solid 0.075 g (8%). ¹H NMR (400 MHz, CDCl3) δ 7.32 (s, 1H), 5.97 (bs, 2H), 5.39 (q, 1H), 5.21-5.07 (m, 2H), 4.40-3.20 (m, 5H), 3.19 (s, 3H), 2.86-2.75 (m, 1H), 2.57-2.43 (m, 1H), 2.14 (s, 3H), 1.59 (d, 3H), 1.55 (d, 3H), 1.50 (d, 3H), 1.41 (d, 3H), 1.33 (t, 3H); m/z [M+H]⁺ 652.5.

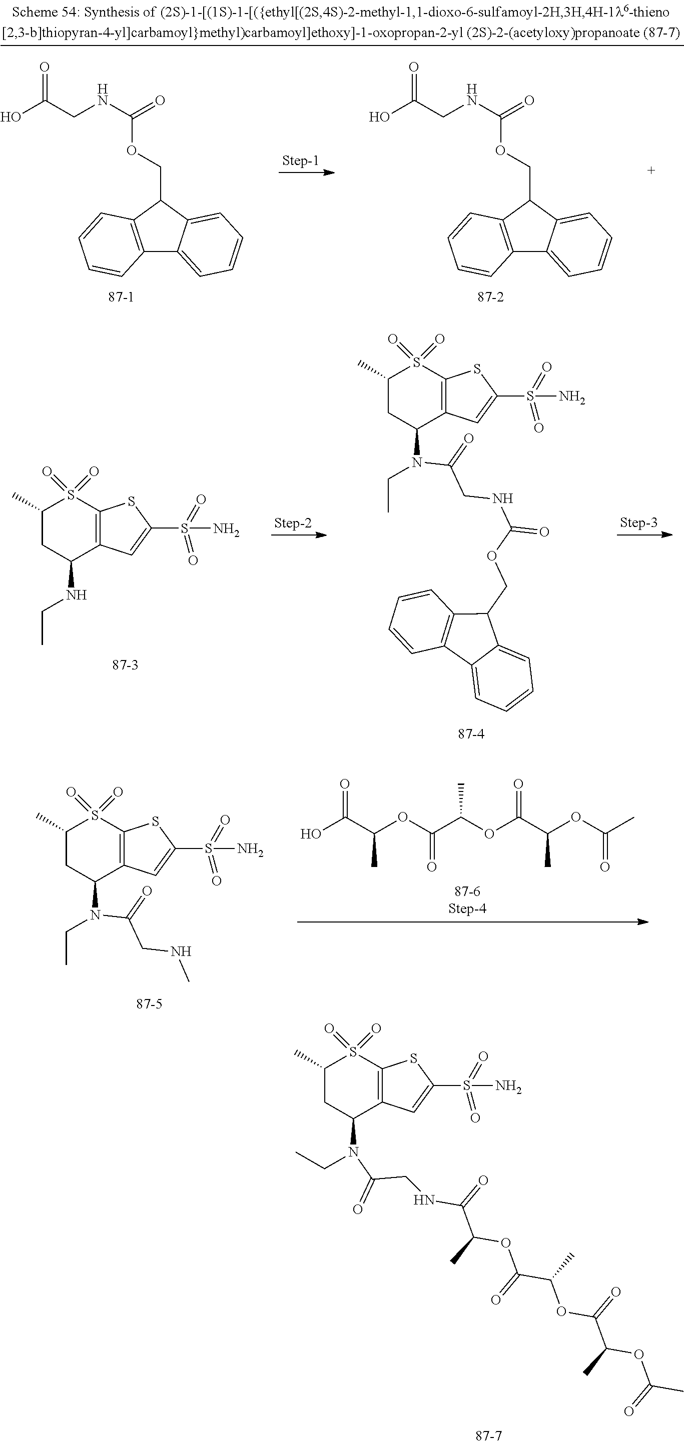

Step-1 & 2: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate (87-4): To a solution of ({[(9H-fluoren-9-yl)methoxy]carbonyl}amino)acetic acid 87-1 (1.2 g, 4.1 mmol) in dichloromethane (20 mL), were added oxalyl chloride (0.7 mL, 8.2 mmol) and N,N-dimethylformamide (0.1 mL) at 0° C. The reaction mixture was allowed to stir at room temperature over a period of 30 min and concentrated the reaction mixture to dryness under nitrogen atmosphere. The residue was diluted with dichloromethane (100 mL) and added N,N-diisopropylethylamine (0.97 mL, 5.5 mmol) followed by dorzolamide 87-3 (1.0 g, 2.7 mmol) at 0° C. The reaction mixture was allowed to stir at room temperature over a period of 1 h. The resulting reaction mass was quenched with water (50 mL), extracted with dichloromethane (2×100 mL), organic layer was dried over sodium sulphate and concentrated under reduced pressure. The crude product obtained upon evaporation of volatiles was purified by silica gel column chromatography to obtain product 87-4 as a white solid 0.56 g (35%).

Step-3: Preparation of 2-amino-N-ethyl-N-[(4S,6S)-6-methyl-7,7-dioxo-2-sulfamoyl-4,5,6,7-tetrahydro-7l6-thieno[2,3-b]thiopyran-4-yl]acetamide (87-5): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate 87-4 (0.43 g, 0.0074 mmol) in dichloromethane (5 V) was added piperidine (0.39 mL, 3.7 mmol) at 0° C. The reaction mixture was allowed to stir at 25-30° C. over a period of 24 hours. The resulting reaction mixture was concentrated under reduced pressure to obtain compound 87-5 as an off white solid 0.25 g (40%). The crude compound was taken forward to next step without any purification.

Step-4: Preparation of (2S)-1-[(1S)-1-[({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamoyl]ethoxy]-1-oxopropan-2-yl (2S)-2-(acetyloxy)propanoate (87-7): To a solution of (2S)-2-{[(2S)-2-{[(2S)-2-(acetyloxy)propanoyl]oxy}propanoyl]oxy}propanoic acid 87-6 (0.48 g, 1.90 mmol) in dichloromethane (10 mL) were added EDC.HCl (0.40 g, 2.3 mmol), N,N-Diisopropylethylamine (0.42 mL, 2.61 mmol) and 2-amino-N-ethyl-N-[(4S,6S)-6-methyl-7,7-dioxo-2-sulfamoyl-4,5,6,7-tetrahydro-7l6-thieno[2,3-b]thiopyran-4-yl]acetamide 5 (0.5 g, 1.31 mmol) at 0° C. The reaction mixture was allowed to stir at 25-30° C. over a period of 2 h. The resulting reaction mass was quenched with water (50 mL), extracted with ethyl acetate (100×2 mL), dried over sodium sulphate and concentrated under reduced pressure. The crude compound was purified by reverse phase column chromatography to obtain product 87-7 as a white solid 0.19 mg (22%). ¹H NMR (400 MHz, DMSO-d6) δ 8.27-8.15 (m, 1H), 8.07 and 8.03 (2bs, 2H), 7.41 and 7.27 (2s, 1H), 5.31-4.97 (m, 4H), 4.13-3.85 (m, 3H), 3.52-3.10 (m, 2H), 2.87-2.60 (m, 1H), 2.5-2.34 (m, 1H), 2.07 (s, 3H), 1.54-1.27 (m, 12H), 1.19 and 1.01 (2t, 3H); m/z [M+H]⁺ 640.4.

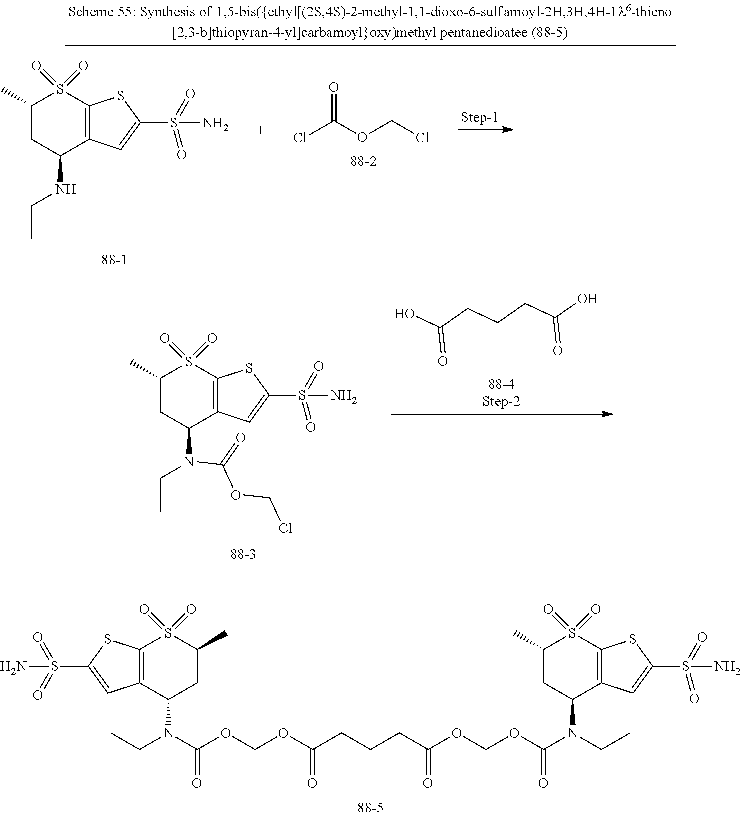 [SMILES: CCN[C@H]1C[C@H](C)S(=O)(=O)c2sc(S(N)(=O)=O)cc21 ; O=C(Cl)OCCl ; CCN(C(=O)OCCl)[C@H]1C[C@H](C)S(=O)(=O)c2sc(S(N)(=O)=O)cc21 ; O=C(O)CCCC(=O)O ; CCN(C(=O)OCOC(=O)CCCC(=O)OCOC(=O)N(CC)[C@H]1C[C@H](C)S(=O)(=O)c2sc(S(N)(=O)=O)cc21)[C@H]1C[C@H](C)S(=O)(=O)c2sc(S(N)(=O)=O)cc21]

Step-1: Preparation chloromethyl N-ethyl-N-[(2S,4S)-2-methyl-1,1-dioxo-6-sulfamoyl-2H,3H,4H-1λ⁶-thieno[2,3-b]thiopyran-4-yl]carbamate (88-3): To a solution of dorzolamide 88-1 (1.4 g, 3.88 mmol) in dichloromethane (25 V) was added N,N-diisopropylethylamine (1.41 mL, 7.7 mmol) at 25-30° C. After 30 min, chloromethyl carbonochloridate (0.38 g, 4.2 mmol) was added at 0° C. The reaction mixture was allowed to stir at 0-5° C. over a period of 1 h. The resulting reaction mass was diluted with ethyl acetate (200 mL) and washed with water (100 mL×2), organic layer was dried over sodium sulfate and concentrated under reduced pressure to obtain compound 88-3 as an off white solid 0.75 g (46%). The crude compound was taken forward to next step without any purification

Step-2: Preparation of 1,5-bis({ethyl[(2S,4S)-2-methyl-1,1-dioxo-6-sulfamoyl-2H,3H,4H-1λ⁶-thieno[2,3-b]thiopyran-4-yl]carbamoyl}oxy)methyl pentanedioate (88-5): To a solution of chloromethyl ethyl[(4S,6S)-6-methyl-7,7-dioxo-2-sulfamoyl-4,5,6,7-tetrahydro-7l6-thieno[2,3-b]thiopyran-4-yl]carbamate 88-3 (0.5 g, 1.2 mmol) in tetrahydrofuran (3 V) were added sodium iodide (0.26 g, 1.80 mmol), pentanedioic acid (0.23mg, 1.8 mmol) and N,N-diisopropylethylamine (0.43 mL, 2.4 mmol) at 28-30° C. The reaction mixture was allowed to stir at 55° C. over a period of 7 h. The resulting reaction mass was diluted with ethyl acetate (180 mL) and washed with water (50 mL×2), organic layer was dried over sodium sulfate and concentrated under reduced pressure. The crude compound was purified by reverse phase column chromatography to obtain product 88-5 as a white solid 0.05 g (5%). ¹H NMR (400 MHz, DMSO-d6/TFA) δ 8.07 (bs, 4H), 7.30 (s, 2H), 5.73-5.46 (m, 4H), 5.13-4.93 (m, 2H), 3.96-3.74 (m, 2H), 3.4-3.0 (m, 4H), 2.87-2.70 (m, 2H), 2.5-2.28 (m, 6H), 1.82-1.66 (m, 2H), 1.42-1.32 (m, 6H) 1.15-1.03 (m, 6H); m/z [M−H]⁻ 891.1.

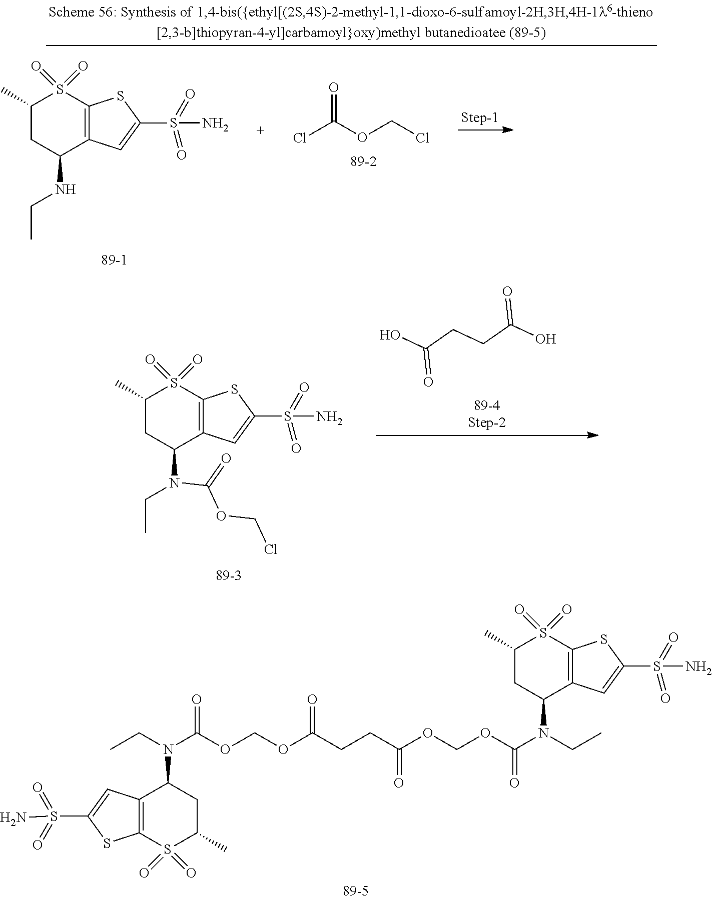 [SMILES: CCN[C@H]1C[C@H](C)S(=O)(=O)c2sc(S(N)(=O)=O)cc21 ; O=C(Cl)OCCl ; CCN(C(=O)OCCl)[C@H]1C[C@H](C)S(=O)(=O)c2sc(S(N)(=O)=O)cc21 ; O=C(O)CCC(=O)O ; CCN(C(=O)OCOC(=O)CCC(=O)OCOC(=O)N(CC)[C@H]1C[C@H](C)S(=O)(=O)c2sc(S(N)(=O)=O)cc21)[C@H]1C[C@H](C)S(=O)(=O)c2sc(S(N)(=O)=O)cc21]

Step-1: Preparation chloromethyl N-ethyl-N-[(2S,4S)-2-methyl-1,1-dioxo-6-sulfamoyl-2H,3H,4H-1λ⁶-thieno[2,3-b]thiopyran-4-yl]carbamate (89-3): To a solution of dorzolamide 89-1 (1.4 g, 3.88 mmol) in dichloromethane (25 V) was added N,N-diisopropylethylamine (1.41 mL, 7.7 mmol) at 25-30° C. After 30 min, chloromethyl carbonochloridate (0.38 g, 4.2 mmol) was added at 0° C. The reaction mixture was allowed to stir at 0-5° C. over a period of 1 h. The resulting reaction mass was diluted with ethyl acetate (200 mL) and washed with water (100 mL×2), organic layer was dried over sodium sulfate and concentrated under reduced pressure to obtain compound 89-3 as an off white solid 0.75 g (46%). The crude compound was taken forward to next step without any purification

Step-2: Preparation of 1,4-bis({ethyl[(2S,4S)-2-methyl-1,1-dioxo-6-sulfamoyl-2H,3H,4H-1λ⁶-thieno[2,3-b]thiopyran-4-yl]carbamoyl}oxy)methyl butanedioate (89-5): To a solution of chloromethyl ethyl[(4S,6S)-6-methyl-7,7-dioxo-2-sulfamoyl-4,5,6,7-tetrahydro-7l6-thieno[2,3-b]thiopyran-4-yl]carbamate 89-3 (0.3 g, 0.72 mmol) in tetrahydrofuran (3 V) were added sodium iodide (0.16 g, 1.08 mmol), butanedioic acid (0.12mg, 1.08 mmol) and N,N-diisopropylethylamine (0.26 mL, 1.4 mmol) at 28-30° C. The reaction mixture was allowed to stir at 55° C. over a period of 7 h. The resulting reaction mass was diluted with ethyl acetate (180 mL) and washed with water (50 mL×2), organic layer was dried over sodium sulfate and concentrated under reduced pressure. The crude compound was purified by reverse phase column chromatography to obtain product 89-5 as a white solid 0.025 g (3%). ¹H NMR (400 MHz, DMSO-d6/TFA) δ 8.05 (bs, 4H), 7.31 (s, 2H), 5.73-5.45 (m, 4H), 5.12-4.95 (m, 2H), 3.96-3.78 (m, 2H), 3.4-3.0 (m, 4H), 2.88-2.71 (m, 2H), 2.69-2.51 (m, 4H), 2.51-2.39 (m, 4H), 1.42-1.33 (m, 6H) 1.13-1.03 (m, 6H); m/z [M+Na]⁺ 901.2.

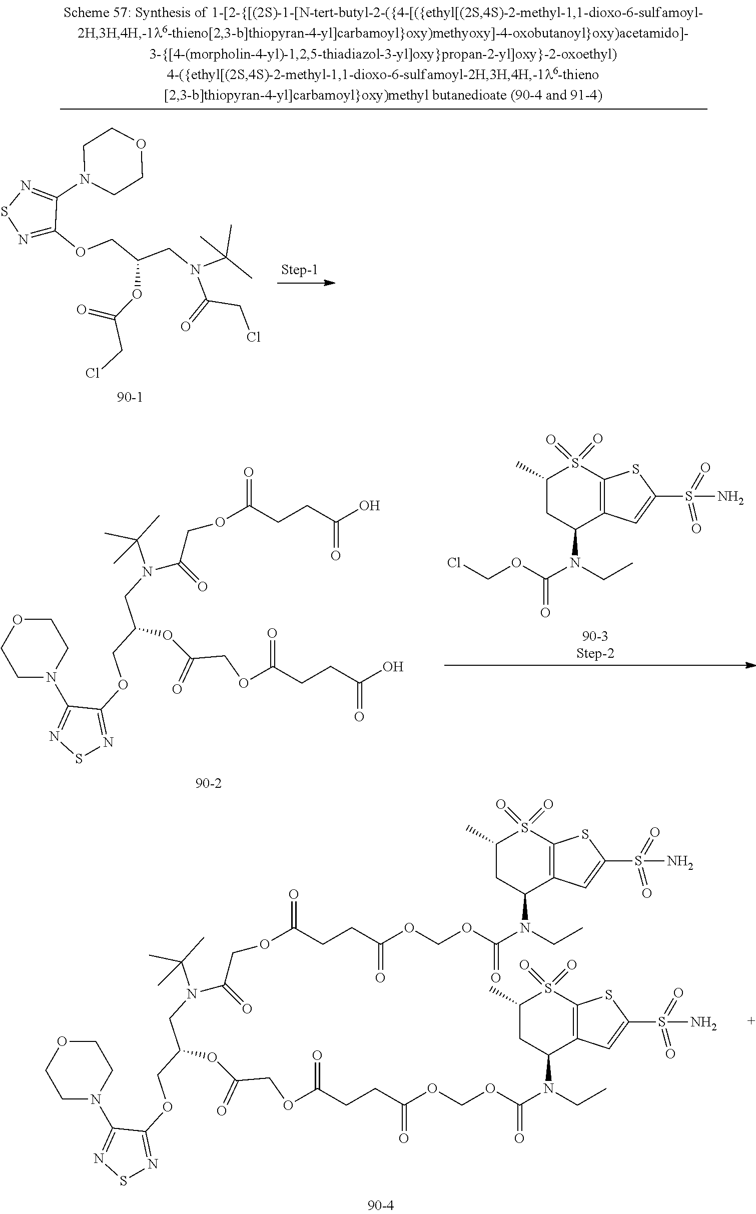 [SMILES: CC(C)(C)N(C[C@@H](COc1nsnc1N1CCOCC1)OC(=O)CCl)C(=O)CCl ; CC(C)(C)N(C[C@@H](COc1nsnc1N1CCOCC1)OC(=O)COC(=O)CCC(=O)O)C(=O)COC(=O)CCC(=O)O ; CCN(C(=O)OCCl)[C@H]1C[C@H](C)S(=O)(=O)c2sc(S(N)(=O)=O)cc21 ; CCN(C(=O)OCOC(=O)CCC(=O)OCC(=O)O[C@H](COc1nsnc1N1CCOCC1)CN(C(=O)COC(=O)CCC(=O)OCOC(=O)N(CC)[C@H]1C[C@H](C)S(=O)(=O)c2sc(S(N)(=O)=O)cc21)C(C)(C)C)[C@H]1C[C@H](C)S(=O)(=O)c2sc(S(N)(=O)=O)cc21]

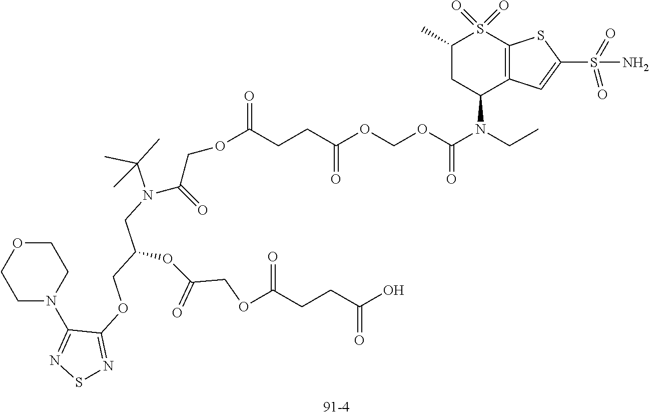 [SMILES: CCN(C(=O)OCOC(=O)CCC(=O)OCC(=O)N(C[C@@H](COc1nsnc1N1CCOCC1)OC(=O)COC(=O)CCC(=O)O)C(C)(C)C)[C@H]1C[C@H](C)S(=O)(=O)c2sc(S(N)(=O)=O)cc21]

Step-1: Preparation of 4-(2-{[(2S)-1-{N-tert-butyl-2-[(3-carboxypropanoyl)oxy]acetamido}-3-{[4-(morpholin-4-yl)-1,2,5-thiadiazol-3-yl]oxy}propan-2-yl]oxy}-2-oxoethoxy)-4-oxobutanoic acid (90-2): To a solution of (2S)-1-(N-tert-butyl-2-chloroacetamido)-3-{[4-(morpholin-4-yl)-1,2,5-thiadiazol-3-yl]oxy}propan-2-yl 2-chloroacetate 90-1 (5.0 g, 10.68 mmol) in N,N-dimethylformamide (3 V) were added trimethylamine (5.9 mL, 42.64 mmol), NaI (3.17 g, 21.32 mmol) and succinic acid (12.57 g, 106.6 mmol) at 0° C. The reaction mixture was allowed to stir at 55° C. over a period of 16 h. The resulting reaction mass was quenched with water (200 mL), extracted with ethyl acetate (400 mL), dried over sodium sulfate and concentrated under reduced pressure. The crude product obtained upon evaporation of volatiles was purified by reverse phase column chromatography to obtain product 90-2 as a brown color wax, 2.5 g (37%).

Step-2: Preparation of -(2-{[(2S)-1-[N-tert-butyl-2-({4-[({ethyl[(2S,4S)-2-methyl-1,1-dioxo-6-sulfamoyl-2H,3H,4H-1λ⁶-thieno[2,3-b]thiopyran-4-yl]carbamoyl}oxy)methoxy]-4-oxobutanoyl}oxy)acetamido]-3-{[4-(morpholin-4-yl)-1,2,5-thiadiazol-3-yl]oxy}propan-2-yl]oxy}-2-oxoethyl) 4-({ethyl[(2S,4S)-2-methyl-1,1-dioxo-6-sulfamoyl-2H,3H,4H-1λ⁶-thieno[2,3-b]thiopyran-4-yl]carbamoyl}oxy)methyl butanedioate (90-4 and 91-4): To a solution of 4-(2-{[(2S)-1-{N-tert-butyl-2-[(3-carboxypropanoyl)oxy]acetamido}-3-{[4-(morpholin-4-yl)-1,2,5-thiadiazol-3-yl]oxy}propan-2-yl]oxy}-2-oxoethoxy)-4-oxobutanoic acid 90-2 (0.2 g, 0.316 mmol) in N,N-dimethylformamide (3 V) were added triethylamine (0.22 mL, 1.58 mmol), chloromethyl N-ethyl-N-[(2S,4S)-2-methyl-1,1-dioxo-6-sulfamoyl-2H,3H,4H-1λ⁶-thieno[2,3-b]thiopyran-4-yl]carbamate 90-3 (0.65 g, 1.58 mmol) and sodium iodide (0.26 g, 1.73 mmol) at 25-28° C. The reaction mixture was allowed to stir at 55° C. over a period of 6 h. The resulting reaction mass was diluted with ethyl acetate (200 mL), washed with water (50 mL×2), organic layer was dried over sodium sulfate and concentrated under reduced pressure. The crude compound was purified by preparative HPLC to obtain product 90-4 (white solid, 34 mg, 7.7%) and 91-4 (white solid, 80 mg, 25%).

90-4: ¹H NMR (400 MHz, DMSO-d6/TFA) δ 8.06 (bs, 4H), 7.31 (s, 2H), 5.73-5.35 (m, 5H), 5.12-4.36 (m, 8H), 3.95-3.76 (m, 2H), 3.70-3.55 (m, 6H), 3.44-3.03 (m, 8H), 2.82-2.54 (m, 10H), 2.5-2.4 (m, 2H), 1.41-1.24 (m, 15H) 1.15-1.02 (m, 6H); m/z [M+H]⁺ 1393.5.

91-4: ¹H NMR (400 MHz, DMSO-d6/TFA) δ 8.06 (bs, 2H), 7.32 (s, 1H), 5.73-5.38 (m, 3H), 5.13-4.38 (m, 7H), 3.96-3.77 (m, 1H), 3.72-3.54 (m, 6H), 3.44-3.04 (m, 6H), 2.85-2.54 (m, 7H), 2.5-2.4 (m, 3H), 1.41-1.27 (m, 12H) 1.15-1.03 (m, 3H); m/z [M+H]⁺ 1013.4.

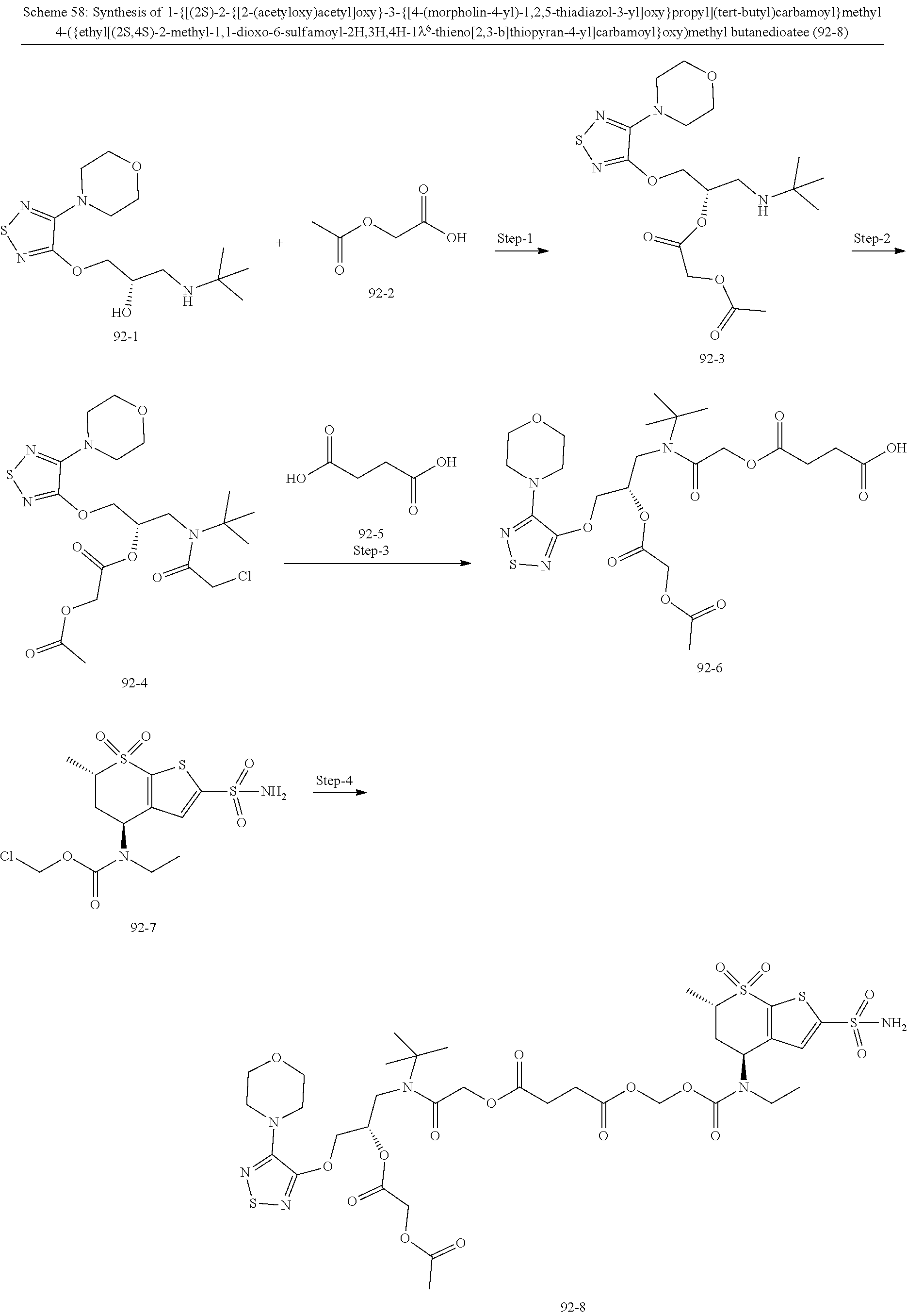

Step 1: Preparation of (2S)-1-(tert-butylamino)-3-{[4-(morpholin-4-yl)-1,2,5-thiadiazol-3-yl]oxy}propan-2-yl 2-(acetyloxy)acetate (92-3): To a solution of timolol 92-1 (5.0 g, 15.82 mmol) in dichloromethane (50 mL) were added 2-acetoxyacetic acid 92-2 (2.17 g, 23.7 mmol), EDC.HCl (6.03 g, 31.6 mmol) and 4-dimethylaminopyridine (0.19 g 1.58 mmol) at 0° C. The reaction mixture was allowed to stir at 25-30° C. over a period of 2 h. The resulting reaction mass was quenched with water (200 mL), extracted with ethyl acetate (250 mL×2), dried over sodium sulfate and concentrated under reduced pressure to obtain 3 as a colorless wax, 4.0 g (63%).The crude compound 92-3 was taken as such into next step without any purification.

Step: 2: Preparation of (2S)-1-(N-tert-butyl-2-chloroacetamido)-3-{[4-(morpholin-4-yl)-1,2,5-thiadiazol-3-yl]oxy}propan-2-yl 2-(acetyloxy)acetate (92-4): To a solution (2S)-1-(tert-butylamino)-3-{[4-(morpholin-4-yl)-1,2,5-thiadiazol-3-yl]oxy}propan-2-yl 2-(acetyloxy)acetate 92-3 (4.5 g, 10.81 mmol) in dichloromethane (50 mL) were added trimethylamine (3.03 mL, 21.6 mmol), 4-dimethylaminopyridine (0.13 g 1.08 mmol) and chloroacetylchloride (1.15 mL, 14.0 mmol) at 0° C. The reaction mixture was allowed to stir at 25-30° C. over a period of 2 h. The resulting reaction mass was quenched with water (150 mL), extracted with ethyl acetate (300 mL), dried over sodium sulfate and concentrated under reduced pressure. The crude product obtained upon evaporation of volatiles was purified through silica gel (230-400 mesh) column chromatography to obtain product 92-4 as a pale brown wax, 2.5 g (47%).

Step: 3: Preparation of (4-({[(2S)-2-{[2-(acetyloxy)acetyl]oxy}-3-{[4-(morpholin-4-yl)-1,2,5-thiadiazol-3-yl]oxy}propyl](tert-butyl)carbamoyl}methoxy)-4-oxobutanoic acid (92-6): To a solution of (2S)-1-(N-tert-butyl-2-chloroacetamido)-3-{[4-(morpholin-4-yl)-1,2,5-thiadiazol-3-yl]oxy}propan-2-yl 2-(acetyloxy)acetate 92-4 (0.5 g, 1.03 mmol) in N,N-dimethylformamide (3V) were added sodium iodide (0.15 g, 1.03 mmol), butanedioic acid 92-5 (0.95 mg, 8.13 mmol) and triethylamine (0.29 mL, 2.06 mmol) at 26-28° C. The reaction mixture was allowed to stir at 55° C. over a period of 16 h. The resulting reaction mass was diluted with ethyl acetate (100 mL) and washed with water (50 mL×2), organic layer was dried over sodium sulfate and concentrated under reduced pressure. The crude product obtained upon evaporation of volatiles was purified by reverse phase column chromatography to obtain product as 92-6 as a pale yellow wax, 0.15 g (25.8%).

Step: 4: Preparation of 1-{[(2S)-2-{[2-(acetyloxy)acetyl]oxy}-3-{[4-(morpholin-4-yl)-1,2,5-thiadiazol-3-yl]oxy}propyl](tert-butyl)carbamoyl}methyl 4-({ethyl[(2S,4S)-2-methyl-1,1-dioxo-6-sulfamoyl-2H,3H,4H-1λ⁶-thieno[2,3-b]thiopyran-4-yl]carbamoyl}oxy)methyl butanedioate (92-8): To a solution chloromethyl N-ethyl-N-[(2S,4S)-2-methyl-1,1-dioxo-6-sulfamoyl-2H,3H,4H-1λ⁶-thieno[2,3-b]thiopyran-4-yl]carbamate 92-7 (0.65 g, 1.56 mmol) in N,N-dimethylformamide (3 V) were added sodium iodide (0.23 g, 1.56 mmol), 4-({[(2S)-2-{[2-(acetyloxy)acetyl]oxy}-3-{[4-(morpholin-4-yl)-1,2,5-thiadiazol-3-yl]oxy}propyl](tert-butyl)carbamoyl}methoxy)-4-oxobutanoic acid 92-6 (0.3 g, 0.522 mmol) and triethylamine (0.25 mL, 1.82 mmol) at 25-28° C. The reaction mixture was allowed to stir at 55° C. over a period of 6 h. The resulting reaction mass was diluted with ethyl acetate (100 mL) and washed with water (50 mL×2), organic layer was dried over sodium sulfate and concentrated under reduced pressure. The crude compound was purified by preparative HPLC to obtain product 92-8 as a white solid, 58 mg (11%). ¹H NMR (400 MHz, DMSO-d6/TFA) δ 8.06 (bs, 2H), 7.32 (s, 1H), 5.72-5.36 (m, 3H), 5.12-4.37 (m, 7H), 3.94-3.76 (m, 1H), 3.71-3.52 (m, 6H), 3.43-3.03 (m, 6H), 2.83-2.54 (m, 5H), 2.5-2.4 (m, 1H), 2.08 (s, 3H) 1.41-1.28 (m, 12H) 1.15-1.02 (m, 3H); m/z [M+H]⁺ 955.3.

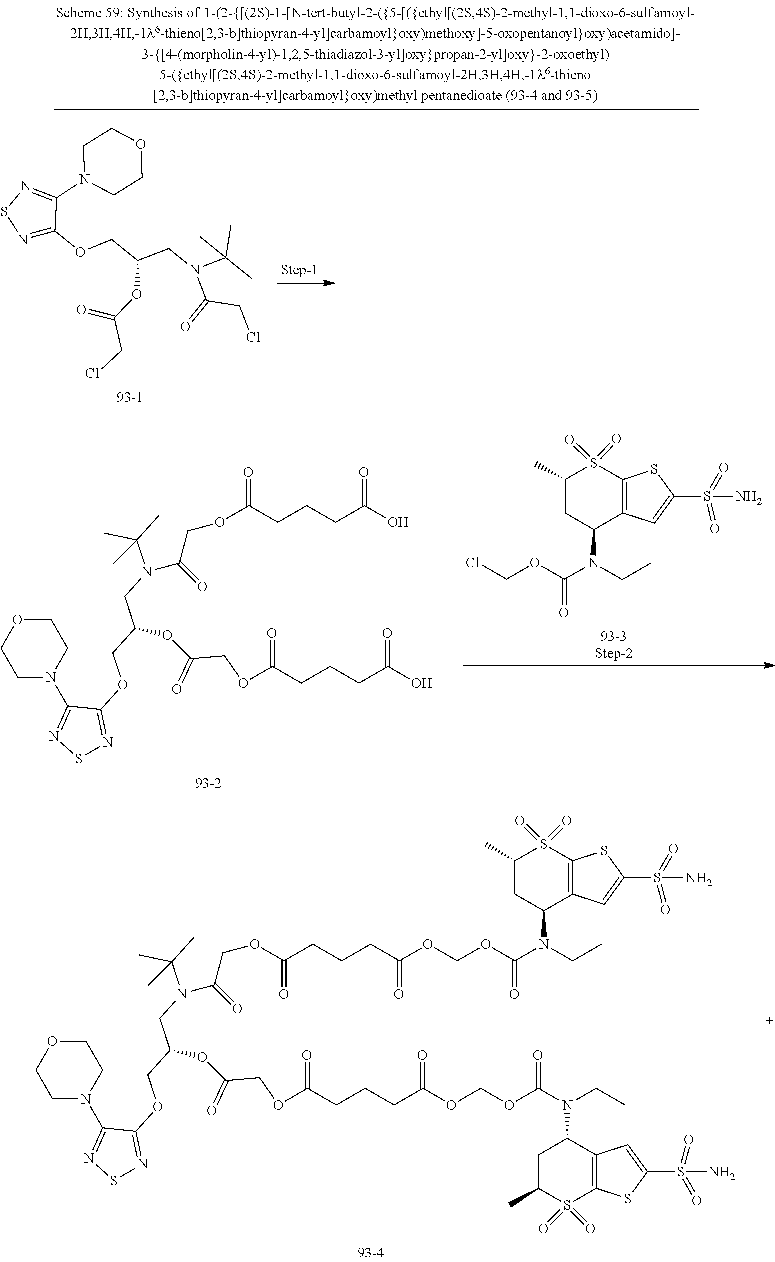 [SMILES: CC(C)(C)N(C[C@@H](COc1nsnc1N1CCOCC1)OC(=O)CCl)C(=O)CCl ; CC(C)(C)N(C[C@@H](COc1nsnc1N1CCOCC1)OC(=O)COC(=O)CCCC(=O)O)C(=O)COC(=O)CCCC(=O)O ; CCN(C(=O)OCCl)[C@H]1C[C@H](C)S(=O)(=O)c2sc(S(N)(=O)=O)cc21 ; CCN(C(=O)OCOC(=O)CCCC(=O)OCC(=O)O[C@H](COc1nsnc1N1CCOCC1)CN(C(=O)COC(=O)CCCC(=O)OCOC(=O)N(CC)[C@H]1C[C@H](C)S(=O)(=O)c2sc(S(N)(=O)=O)cc21)C(C)(C)C)[C@H]1C[C@H](C)S(=O)(=O)c2sc(S(N)(=O)=O)cc21]

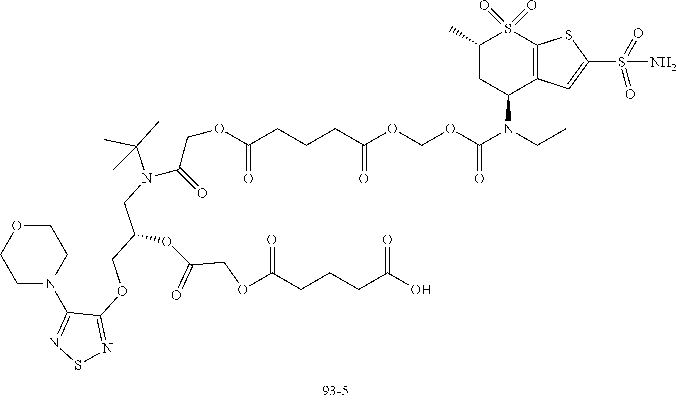 [SMILES: CCN(C(=O)OCOC(=O)CCCC(=O)OCC(=O)N(C[C@@H](COc1nsnc1N1CCOCC1)OC(=O)COC(=O)CCCC(=O)O)C(C)(C)C)[C@H]1C[C@H](C)S(=O)(=O)c2sc(S(N)(=O)=O)cc21]

Step 1: Preparation of 5-(2-{[(2S)-1-{N-tert-butyl-2-[(4-carboxybutanoyl)oxy]acetamido}-3-{[4-(morpholin-4-yl)-1,2,5-thiadiazol-3-yl]oxy}propan-2-yl]oxy}-2-oxoethoxy)-5-oxopentanoic acid (93-2): To a solution of (2S)-1-(N-tert-butyl-2-chloroacetamido)-3-{[4-(morpholin-4-yl)-1,2,5-thiadiazol-3-yl]oxy}propan-2-yl 2-chloroacetate 93-1 (3.0 g, 6.39 mmol) in N,N-dimethylformamide (9 mL) were added trimethylamine (3.4 mL, 25.5 mmol), NaI (1.9 g, 12.7 mmol) and glutaric acid (8.4 g, 63.9 mmol) at 0° C. The reaction mixture was allowed to stir at 55° C. over a period of 16 h. The resulting reaction mass was quenched with water (150 mL), extracted with ethyl acetate (300 mL), dried over sodium sulfate and concentrated under reduced pressure. The crude product obtained upon evaporation of volatiles was purified by reverse phase column chromatography to obtain product 93-2 as a brown color wax, 2.7 g (64%).

Step 2: Preparation of 1-(2-{[(2S)-1-[N-tert-butyl-2-({5-[({ethyl[(2S,4S)-2-methyl-1,1-dioxo-6-sulfamoyl-2H,3H,4H-1λ⁶-thieno[2,3-b]thiopyran-4-yl]carbamoyl}oxy)methoxy]-5-oxopentanoyl}oxy)acetamido]-3-{[4-(morpholin-4-yl)-1,2,5-thiadiazol-3-yl]oxy}propan-2-yl]oxy}-2-oxoethyl) 5-({ethyl[(2S,4S)-2-methyl-1,1-dioxo-6-sulfamoyl-2H,3H,4H-1λ⁶-thieno[2,3-b]thiopyran-4-yl]carbamoyl}oxy)methyl pentanedioate (93-4): To a solution of chloromethyl N-ethyl-N-[(2S,4S)-2-methyl-1,1-dioxo-6-sulfamoyl-2H,3H,4H-1λ⁶-thieno[2,3-b]thiopyran-4-yl]carbamate 93-3 (0.12 g, 0.302 mmol) in tetrahydrofuran (10 V) were added sodium iodide (0.054 g, 0.36 mmol), 5-(2-{[(2S)-1-{N-tert-butyl-2-[(4-carboxybutanoyl)oxy]acetamido}-3-{[4-(morpholin-4-yl)-1,2,5-thiadiazol-3-yl]oxy}propan-2-yl]oxy}-2-oxoethoxy)-5-oxopentanoic acid 93-2 (0.2 g, 0.302 mmol) and N,N-diisopropylethylamine (0.10 mL, 0.604 mmol) at 0° C. The reaction mixture was allowed to stir at 25-28° C. over a period of 3 hours. The resulting reaction mass was diluted with ethyl acetate (100 mL) and washed with water (50 mL×2), organic layer was dried over sodium sulfate and concentrated under reduced pressure. The crude compound was purified by preparative HPLC to obtain product 93-4 (white solid, 0.1 g, 23.2%) and 93-5 (white solid, 70 mg, 22.0%).

Product 93-4: ¹H NMR (400 MHz, DMSO-d6) δ 8.07 (bs, 4H), 7.31 (s, 2H), 5.72-5.35 (m, 5H), 5.11-4.38 (m, 8H), 3.96-3.77 (m, 2H), 3.71-3.54 (m, 6H), 3.44-3.02 (m, 8H), 2.83-2.69 (m, 2H), 2.5-2.30 (m, 10H), 1.86-1.68 (m, 4H), 1.41-1.24 (m, 15H) 1.15-1.04 (m, 6H); m/z [M+H]⁺ 1421.5.

Product 93-5: ¹H NMR (400 MHz, DMSO-d6/TFA) δ 8.06 (bs, 2H), 7.30 (s, 1H), 5.73-5.39 (m, 3H), 5.13-4.40 (m, 7H), 3.96-3.78 (m, 1H), 3.71-3.52 (m, 6H), 3.43-3.05 (m, 6H), 2.85-2.70 (m, 1H), 2.5-2.35 (m, 7H), 2.35-.220 (m, 2H), 1.84-1.67 (m, 4H), 1.41-1.27 (m, 12H), 1.15-1.04 (m, 3H); m/z [M+H]⁺ 1041.4.

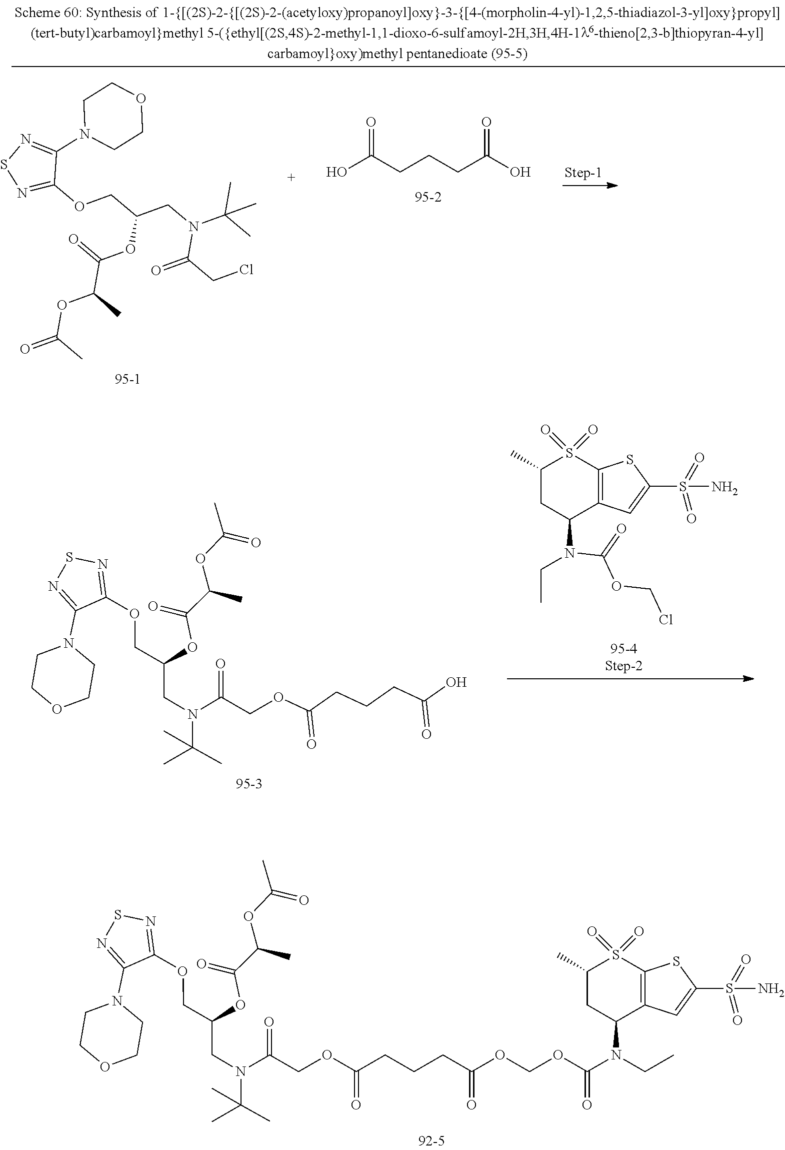 [SMILES: CC(=O)O[C@H](C)C(=O)O[C@H](COc1nsnc1N1CCOCC1)CN(C(=O)CCl)C(C)(C)C ; O=C(O)CCCC(=O)O ; CC(=O)O[C@@H](C)C(=O)O[C@H](COc1nsnc1N1CCOCC1)CN(C(=O)COC(=O)CCCC(=O)O)C(C)(C)C ; CCN(C(=O)OCCl)[C@H]1C[C@H](C)S(=O)(=O)c2sc(S(N)(=O)=O)cc21 ; CCN(C(=O)OCOC(=O)CCCC(=O)OCC(=O)N(C[C@@H](COc1nsnc1N1CCOCC1)OC(=O)[C@H](C)OC(C)=O)C(C)(C)C)[C@H]1C[C@H](C)S(=O)(=O)c2sc(S(N)(=O)=O)cc21]

Step-1: Preparation 5-({[(2S)-2-{[(2S)-2-(acetyloxy)propanoyl]oxy}-3-{[4-(morpholin-4-yl)-1,2,5-thiadiazol-3-yl]oxy}propyl](tert-butyl)carbamoyl}methoxy)-5-oxopentanoic acid (95-3): To a solution of (2S)-1-(N-tert-butyl-2-chloroacetamido)-3-{[4-(morpholin-4-yl)-1,2,5-thiadiazol-3-yl]oxy}propan-2-yl (2R)-2-(acetyloxy)propanoate 95-1 (0.5 g, 1.03 mmol) in N,N-dimethylformamide (3 V) were added sodium iodide (0.15 g, 1.03 mmol), pentanedioic acid 95-2 (0.95 mg, 8.13 mmol) and triethylamine (0.29 mL, 2.06 mmol) at 0° C. The reaction mixture was allowed to stir at 55° C. over a period of 16 hours. The resulting reaction mass was diluted with ethyl acetate (100 mL) and washed with water (50 mL×2), organic layer was dried over sodium sulfate and concentrated under reduced pressure. The crude product obtained upon evaporation of volatiles was purified revers phase column chromatography to obtain product 3 as a colorless wax 0.55 g (68%).

Step-2: Preparation of 1-{[(2S)-2-{[(2S)-2-(acetyloxy)propanoyl]oxy}-3-{[4-(morpholin-4-yl)-1,2,5-thiadiazol-3-yl]oxy}propyl](tert-butyl)carbamoyl}methyl 5-({ethyl[(2S,4S)-2-methyl-1,1-dioxo-6-sulfamoyl-2H,3H,4H-1λ⁶-thieno[2,3-b]thiopyran-4-yl]carbamoyl}oxy)methyl pentanedioate (95-5): To a solution of 5-({[(2S)-2-{[(2S)-2-(acetyloxy)propanoyl]oxy}-3-{[4-(morpholin-4-yl)-1,2,5-thiadiazol-3-yl]oxy}propyl](tert-butyl)carbamoyl}methoxy)-5-oxopentanoic acid 95-3 (1.0 g, 1.66 mmol) in THF (20V) were added chloromethyl N-ethyl-N-[(2S,4S)-2-methyl-1,1-dioxo-6-sulfamoyl-2H,3H,4H-1λ⁶-thieno[2,3-b]thiopyran-4-yl]carbamate 95-4 (1 g, 2.49 mmol), DIPEA (0.61 mL, 3.32 mmol) and NaI (0.371 g, 2.49 mmol) at 25-30° C. The reaction mixture was allowed to stir at 55° C. over a period of 3 h. The resulting reaction mass was diluted with ethyl acetate (300 mL) and washed with water (100 mL×2), organic layer was dried over sodium sulfate and concentrated under reduced pressure. The crude was purified by preparative HPLC to obtain product 95-5 as a white solid 0.5 g (31%). ¹H NMR (400 MHz, DMSO-d6/TFA) δ 8.06 (bs, 2H), 7.32 (s, 1H), 5.72-5.32 (m, 3H), 5.12-4.99 (m, 2H), 4.86-4.73 (m, 2H), 4.61-4.52 (m, 1H), 4.49-4.37 (m, 1H), 3.95-3.76 (m, 1H), 3.72-3.59 (m, 6H), 3.45-3.04 (m, 6H), 2.84-2.66 (m, 1H), 2.5-2.34 (m, 5H), 2.03 (s, 3H), 1.86-1.70 (m, 2H), 1.41-1.27 (m, 15H), 1.15-1.03 (m, 3H); m/z [M−H]⁻ 981.5.

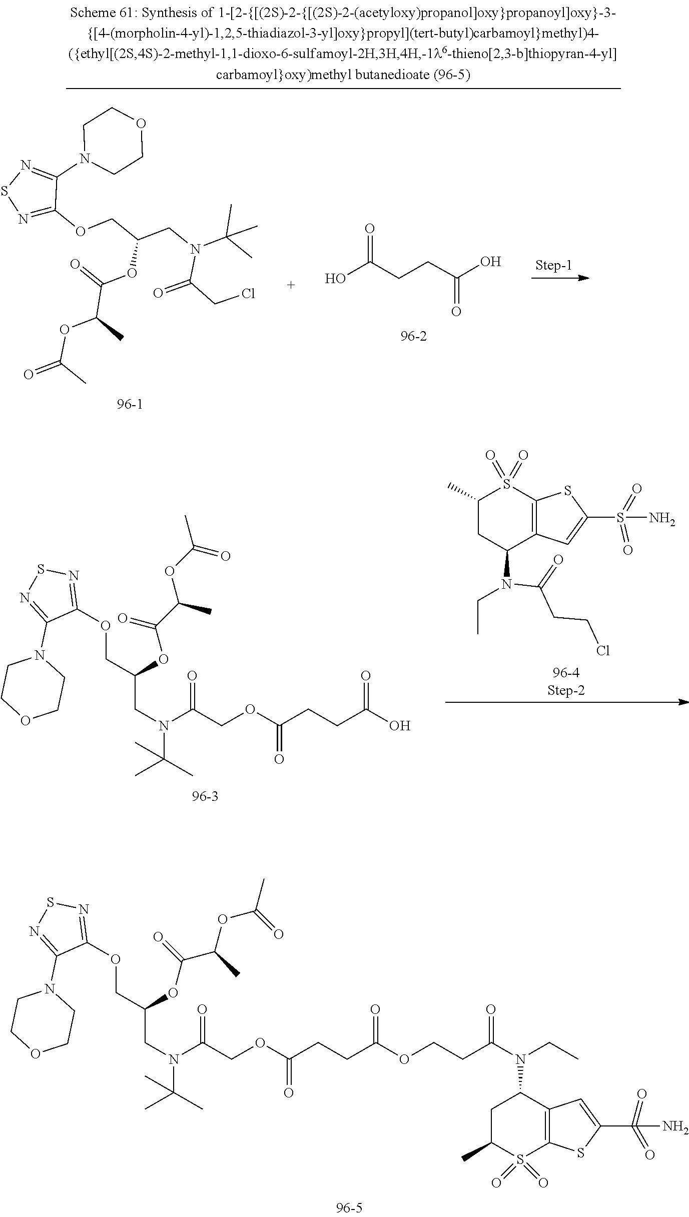

Step-1: Preparation of 4-({[(2S)-2-{[(2S)-2-(acetyloxy)propanoyl]oxy}-3-{[4-(morpholin-4-yl)-1,2,5-thiadiazol-3-yl]oxy}propyl](tert-butyl)carbamoyl}methoxy)-4-oxobutanoic acid (96-3): To a solution of (2S)-1-(N-tert-butyl-2-chloroacetamido)-3-{[4-(morpholin-4-yl)-1,2,5-thiadiazol-3-yl]oxy}propan-2-yl (2R)-2-(acetyloxy)propanoate 96-1 (0.5 g, 1.03 mmol) in N,N-dimethylformamide (3 V) were added sodium iodide (0.15 g, 1.03 mmol), butanedioic acid 96-2 (0.95 mg, 8.13 mmol) and triethylamine (0.29 mL, 2.06 mmol) at 0° C. The reaction mixture was allowed to stir at 55° C. over a period of 16 hours. The resulting reaction mass was diluted with ethyl acetate (100 mL) and washed with water (50 mL×2), organic layer was dried over sodium sulfate and concentrated under reduced pressure. The crude product obtained upon evaporation of volatiles was purified reverse phase column chromatography to obtain product 96-3 as a colorless wax 0.35 g (60%).

Step-2: Preparation of 1-{[(2S)-2-{[(2S)-2-(acetyloxy)propanoyl]oxy}-3-{[4-(morpholin-4-yl)-1,2,5-thiadiazol-3-yl]oxy}propyl](tert-butyl)carbamoyl}methyl 4-({ethyl[(2S,4S)-2-methyl-1,1-dioxo-6-sulfamoyl-2H,3H,4H-1λ⁶-thieno[2,3-b]thiopyran-4-yl]carbamoyl}oxy)methyl butanedioate (96-5): To a solution of 5 4-({[(2S)-2-{[(2S)-2-(acetyloxy)propanoyl]oxy}-3-{[4-(morpholin-4-yl)-1,2,5-thiadiazol-3-yl]oxy}propyl](tert-butyl)carbamoyl}methoxy)-4-oxobutanoic acid 96-3 (1.0 g, 1.70 mmol) in THF (20V) were added chloromethyl N-ethyl-N-[(2S,4S)-2-methyl-1,1-dioxo-6-sulfamoyl-2H,3H,4H-1λ⁶-thieno[2,3-b]thiopyran-4-yl]carbamate 96-4 (1.0 g, 2.55 mmol), DIPEA (0.62 mL, 3.40 mmol) and NaI (0.380 g, 2.55 mmol) at 25-30° C. The reaction mixture was allowed to stir at 55° C. over a period of 3 h. The resulting reaction mass was diluted with ethyl acetate (200 mL) and washed with water (50 mL×2), organic layer was dried over sodium sulfate and concentrated under reduced pressure. The crude was purified by preparative HPLC to obtain product 96-5 as a white solid 0.24 g (14%). ¹H NMR (400 MHz, DMSO-d6/TFA) δ 8.06 (bs, 2H), 7.32 (s, 1H), 5.71-5.32 (m, 3H), 5.12-4.98 (m, 2H), 4.89-4.72 (m, 2H), 4.60-4.52 (m, 1H), 4.49-4.37 (m, 1H), 3.96-3.76 (m, 1H), 3.72-3.56 (m, 6H), 3.50-3.04 (m, 6H), 2.84-2.56 (m, 5H), 2.5-2.4 (m, 1H), 2.03 (s, 3H), 1.41-1.25 (m, 15H), 1.16-1.02 (m, 3H); m/z [M+H]⁺ 969.3.

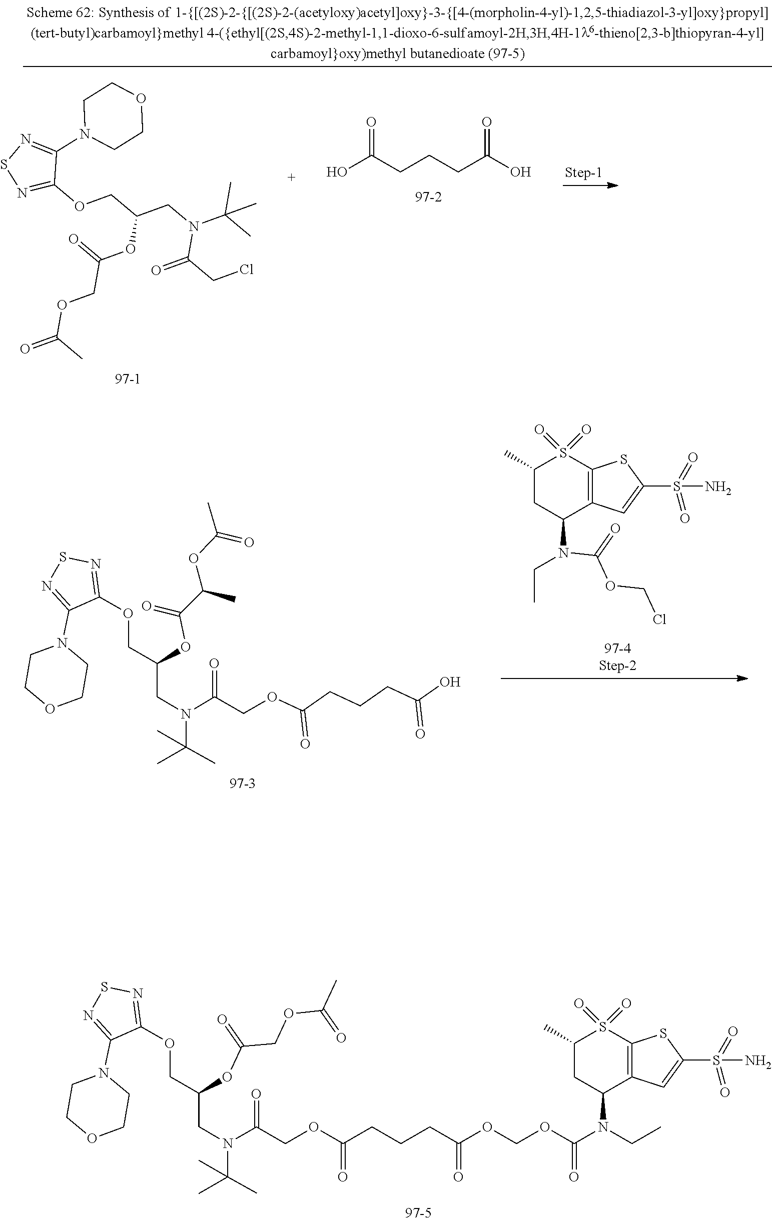 [SMILES: CC(=O)OCC(=O)O[C@H](COc1nsnc1N1CCOCC1)CN(C(=O)CCl)C(C)(C)C ; O=C(O)CCCC(=O)O ; CC(=O)O[C@@H](C)C(=O)O[C@H](COc1nsnc1N1CCOCC1)CN(C(=O)COC(=O)CCCC(=O)O)C(C)(C)C ; CCN(C(=O)OCCl)[C@H]1C[C@H](C)S(=O)(=O)c2sc(S(N)(=O)=O)cc21 ; CCN(C(=O)OCOC(=O)CCCC(=O)OCC(=O)N(C[C@@H](COc1nsnc1N1CCOCC1)OC(=O)COC(C)=O)C(C)(C)C)[C@H]1C[C@H](C)S(=O)(=O)c2sc(S(N)(=O)=O)cc21]

Step-1: Preparation of 4-({[(2S)-2-{[2-(acetyloxy)acetyl]oxy}-3-{[4-(morpholin-4-yl)-1,2,5-thiadiazol-3-yl]oxy}propyl](tert-butyl)carbamoyl}methoxy)-4-oxobutanoic acid (97-3): To a solution of (2S)-1-(N-tert-butyl-2-chloroacetamido)-3-{[4-(morpholin-4-yl)-1,2,5-thiadiazol-3-yl]oxy}propan-2-yl 2-(acetyloxy)acetate 97-1 (0.5 g, 1.03 mmol) in N,N-dimethylformamide (3 V) were added sodium iodide (0.15 g, 1.03 mmol), butanedioic acid 97-2 (0.95 mg, 8.13 mmol) and triethylamine (0.29 mL, 2.06 mmol) at 0° C. The reaction mixture was allowed to stir at 55° C. over a period of 16 h. The resulting reaction mass was diluted with ethyl acetate (100 mL) and washed with water (50 mL×2), organic layer was dried over sodium sulfate and concentrated under reduced pressure. The crude product obtained upon evaporation of volatiles was purified by reverse phase column chromatography to obtain product as 97-3 as a colorless wax 0.3 g (36%).

Step-2: Preparation of 1-{[(2S)-2-{[2-(acetyloxy)acetyl]oxy}-3-{[4-(morpholin-4-yl)-1,2,5-thiadiazol-3-yl]oxy}propyl](tert-butyl)carbamoyl}methyl 5-({ethyl[(2S,4S)-2-methyl-1,1-dioxo-6-sulfamoyl-2H,3H,4H-1λ⁶-thieno[2,3-b]thiopyran-4-yl]carbamoyl}oxy)methyl pentanedioate (97-5): To a solution of 5-({[(2S)-2-{[2-(acetyloxy)acetyl]oxy}-3-{[4-(morpholin-4-yl)-1,2,5-thiadiazol-3-yl]oxy}propyl](tert-butyl)carbamoyl}methoxy)-5-oxopentanoic acid 97-3 (1.0 g, 1.70 mmol) in THF (20V) were added chloromethyl N-ethyl-N-[(2S,4S)-2-methyl-1,1-dioxo-6-sulfamoyl-2H,3H,4H-1λ⁶-thieno[2,3-b]thiopyran-4-yl]carbamate 97-4 (1.0 g, 2.55 mmol), DIPEA (0.62 mL, 3.40 mmol) and NaI (0.380 g, 2.55 mmol) at 25-30° C. The reaction mixture was allowed to stir at 55° C. over a period of 3 h. The resulting reaction mass was diluted with ethyl acetate (300 mL) and washed with water (50 mL×2), organic layer was dried over sodium sulfate and concentrated under reduced pressure. The crude compound was purified by preparative HPLC to obtain product 97-5 as a white solid 0.68 g (42%). ¹H NMR (400 MHz, DMSO-d6) δ 8.07 (bs, 2H), 7.30 (s, 1H), 5.74-5.36 (m, 3H), 5.12-4.97 (m, 1H), 4.94-4.85 (m, 1H), 4.74-4.64 (m, 3H), 4.62-4.53 (m, 1H), 4.50-4.39 (m, 1H), 3.95-3.75 (m, 1H), 3.71-3.53 (m, 6H), 3.44-3.04 (m, 6H), 2.84-2.67 (m, 1H), 2.5-2.35 (m, 5H), 2.08 (s, 3H), 1.85-1.69 (s, 2H), 1.41-1.25 (m, 12H), 1.15-1.02 (m, 3H); m/z [M−H]⁻ 967.3.

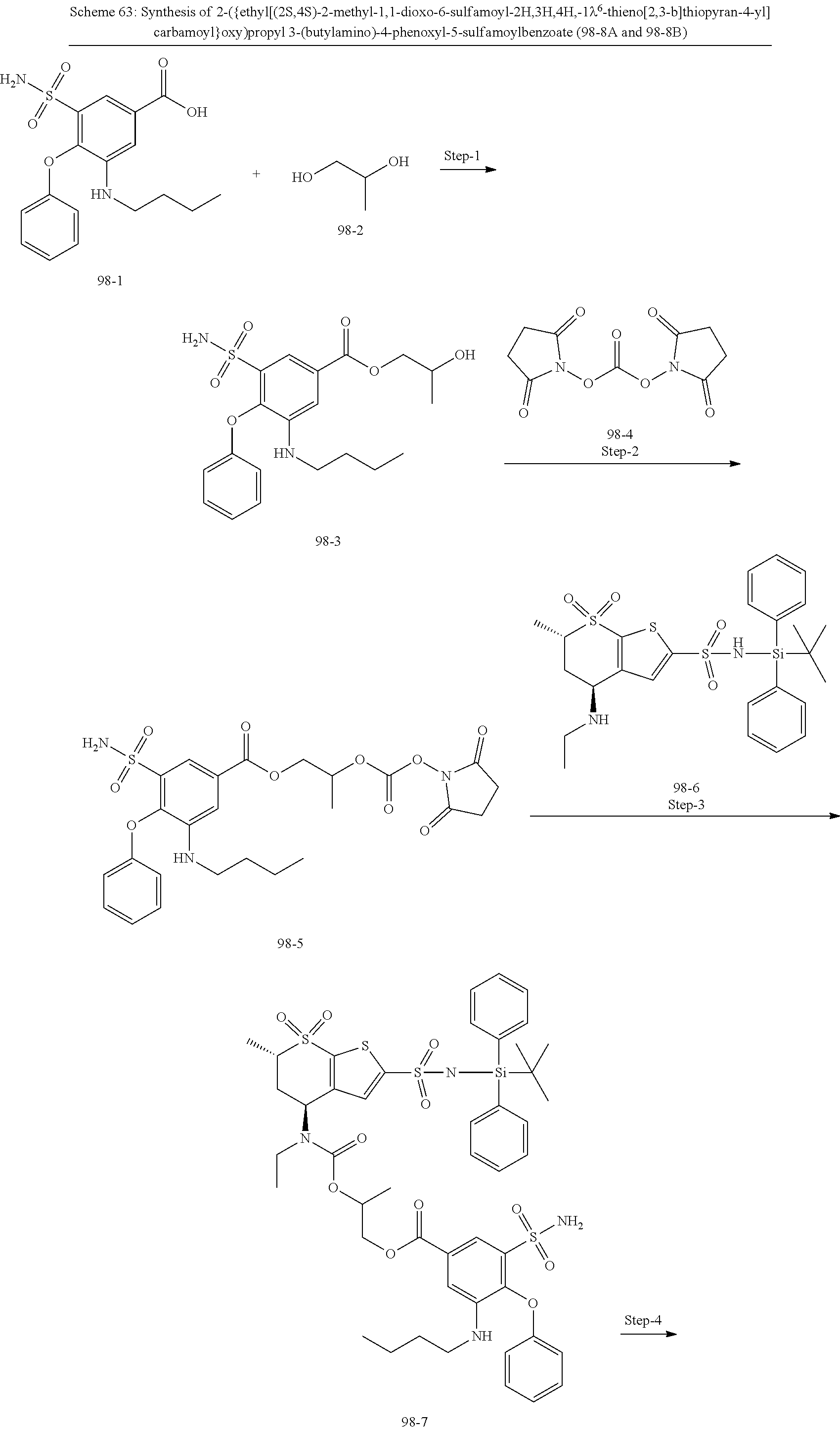

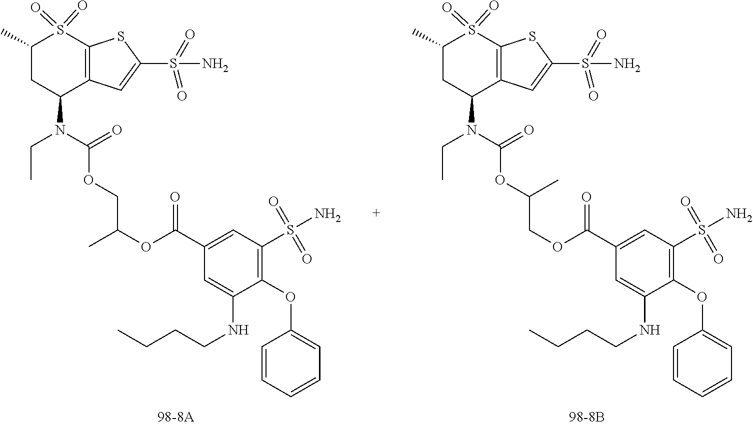 [SMILES: CCCCNc1cc(C(=O)OC(C)COC(=O)N(CC)[C@H]2C[C@H](C)S(=O)(=O)c3sc(S(N)(=O)=O)cc32)cc(S(N)(=O)=O)c1Oc1ccccc1 ; "+" ; CCCCNc1cc(C(=O)OCC(C)OC(=O)N(CC)[C@H]2C[C@H](C)S(=O)(=O)c3sc(S(N)(=O)=O)cc32)cc(S(N)(=O)=O)c1Oc1ccccc1]

Step-1: Preparation of 2-hydroxypropyl 3-(butylamino)-4-phenoxy-5-sulfamoylbenzoate (98-3): To a solution of bumetanide 98- (5.0 g, 13.73 mmol) in THF (50 mL) were added EDC.HCl (3.9 g, 20.5 mmol), HOBt (5.2 g, 13.7 mmol), propylene glycol 98-2 (1.35 g, 17.8 mmol) and 4-Dimethylaminopyridine (0.3 g, 2.74 mmol) at 0-5° C. The reaction mixture was refluxed at 80° C. for 16 h. The resulting reaction mixture was diluted with ethyl acetate (300 mL) and washed with water (2×150 mL). The organic layer was dried over sodium sulphate and concentrated under reduced pressure at 45° C. The crude compound was purified by reverse phase column chromatography to obtain product 98-3 as white solid 2.5 g (43%).

Step-2: Preparation of 2-({[(2,5-dioxopyrrolidin-1-yl)oxy]carbonyl}oxy)propyl 3-(butylamino)-4-phenoxy-5-sulfamoylbenzoate (98-5): To a solution of 2-hydroxypropyl 3-(butylamino)-4-phenoxy-5-sulfamoylbenzoate 98-3 (1.0 g, 2.36 mmol) in tetrahydrofuran (10 mL) was added Pyridine (0.8 mL, 8.26 mmol), bis(2,5-dioxopyrrolidin-1-yl) carbonate (1.8 g, 7.10 mmol) 98-4 and 4-Dimethylaminopyridine (0.057 g, 0.47 mmol) at 0° C. The reaction mixture was stirred at 25-30° C. over a period of 16 h. The resulting reaction mixture was diluted with ethyl acetate (300 mL) and washed with water (2×150 mL). The organic layer was dried over sodium sulphate and concentrated under reduced pressure. The crude product obtained upon evaporation of volatiles was recrystallized using methanol to obtain product 98-5 as a white solid 1.0 g (76%).

Step-3: Preparation of 2-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)propyl 3-(butylamino)-4-phenoxy-5-sulfamoylbenzoate (98-7): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 98-6 (0.3 g, 0.533 mmol) in THF (50 mL) were added pyridine (0.1 mL, 1.06 mmol), 2-({[(2,5-dioxopyrrolidin-1-yl)oxy]carbonyl}oxy)propyl3 -(butylamino)-4-phenoxy-5sulfamoylbenzoate 98-5 (0.3 g, 0.53 mmol) and 4-Dimethylaminopyridine (0.013 g, 0.10 mmol) at 0-5° C. The reaction mixture was stirred at 80° C. over a period of 24 h. The resulting reaction mixture was diluted with ethyl acetate (200 mL) and washed with water (2×100 mL). The organic layer was dried over sodium sulphate and concentrated under reduced pressure to afford 98-7 as a white solid 0.4 g. The crude compound 98-7 was taken as such into next step without any purification.

Step-4: Preparation of 2-({ethyl[(2S,4S)-2-methyl-1,1-dioxo-6-sulfamoyl-2H,3H,4H-1λ⁶-thieno[2,3-b]thiopyran-4-yl]carbamoyl}oxy)propyl 3-(butylamino)-4-phenoxy-5-sulfamoylbenzoate (98-8): To a solution of 2-({[(2S,4S)-6-[(tert-butyldiphenylsilyl)sulfamoyl]-2-methyl-1,1-dioxo-2H,3H,4H-1λ⁶-thieno[2,3-b]thiopyran-4-yl](ethyl)carbamoyl}oxy)propyl 3-(butylamino)-4-phenoxy-5-sulfamoylbenzoate 98-7 (0.4 g, 0.39 mmol) in tetrahydrofuran (5 mL) were added TBAF (0.11 mL, 1M in THF, 0.11 mmol) and acetic acid (0.006 mL, 0.11 mmol) at 0-5° C. The reaction mixture was allowed to stir at 0-5° C. for 30 min. The resulting reaction mixture was diluted with ethyl acetate (200 mL) and washed with water (2×100 mL). The organic layer was dried over sodium sulphate and concentrated under reduced pressure. The crude product obtained upon evaporation of volatiles was purified by preparative HPLC to give products 98-8A and 98-8B as a white solid 90 mg (30%). The two fractions were isolated with the same MS characteristics ([M+H]⁺ 773.3.), but distinct ¹H NMR.

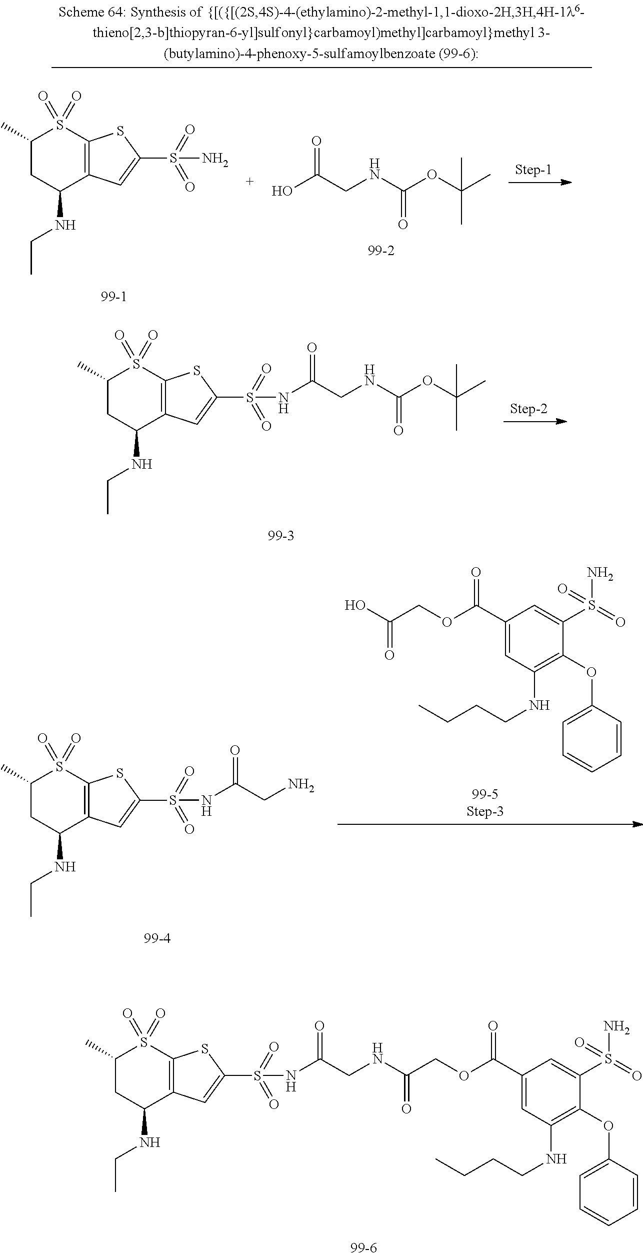

Step-1: Preparation of tert-butyl N-[({[(2S,4S)-4-(ethylamino)-2-methyl-1,1-dioxo-2H,3H,4H-1λ⁶-thieno[2,3-b]thiopyran-6-yl]sulfonyl}carbamoyl)methyl]carbamate (99-3): To a solution of dorzolamide 99-1 (1.0 g, 2.77 mmol, 1 eq) in dichloromethane (20 mL) was added triethylamine (0.78 mL, 5.50 mmol) at 0° C. After 30 min, 2-{[(tert-butoxy)carbonyl]amino}acetic acid 99-2 (0.63 g, 3.61 mmol), EDC.HCl (0.8 g, 4.16 mmol) and 4-dimethylaminopyridine (0.03 g, 0.27 mmol) were added at 0° C. The reaction mixture was allowed to stir at 25-30° C. over a period of 1 h. The resulting reaction mass was quenched with water (200 mL), extracted with dichloromethane (250×2 mL), dried over sodium sulphate and concentrated under reduced pressure. The crude product obtained upon evaporation of volatiles was purified through silica gel (230-400 mesh) column (3% methanol in DCM) to obtain product 99-3 1.1 g (82%).

Step-2: Preparation of 2-amino-N-{[(2S,4S)-4-(ethylamino)-2-methyl-1,1-dioxo-2H,3H,4H-1λ⁶-thieno[2,3-b]thiopyran-6-yl]sulfonyl}acetamide (99-4): To a solution of tert-butyl N-[({[(2S,4S)-4-(ethylamino)-2-methyl-1,1-dioxo-2H,3H,4H-1λ⁶-thieno[2,3-b]thiopyran-6-yl]sulfonyl}carbamoyl)methyl]carbamate 99-3 (1.0 g, 2.07 mmol) in dichloromethane (10 mL), was added TFA (4 mL, 4 V) at 0° C. The reaction mixture was allowed to stir at 0° C. over a period of 1 h. The reaction mass was concentrated under reduced pressure to obtain product 99-4 as pale yellow wax 1.0 g (79%). The crude compound 4 was carried as such into next step without any purification.

Step-3: Preparation of {[({[(2S,4S)-4-(ethylamino)-2-methyl-1,1-dioxo-2H,3H,4H-1λ⁶-thieno[2,3-b]thiopyran-6-yl]sulfonyl}carbamoyl)methyl]carbamoyl}methyl 3-(butylamino)-4-phenoxy-5-sulfamoylbenzoate (99-6): To a solution of 2-amino-N-{[(2S,4S)-4-(ethylamino)-2-methyl-1,1-dioxo-2H,3H,4H-1λ⁶-thieno[2,3-b]thiopyran-6-yl]sulfonyl}acetamide 99-4 (0.9 g, 2.3 mmol) in dichloromethane (20 mL) was added N-methyl morpholine (0.53 mL, 4.7 mmol) at 0° C. After 30 min, 2-[3-(butylamino)-4-phenoxy-5-sulfamoylbenzoyloxy]acetic acid benzyl 2-bromoacetate amine dihydrate 99-5 (1.0 g, 2.3 mmol), EDC.HCl (0.5 g, 2.6 mmol) and 4-dimethylaminopyridine (0.03 g, 0.23 mmol) were added at 0° C. The resulting reaction mixture was allowed to stir at 25-30° C. over a period of 16 h. The reaction mass was quenched with sodium bicarbonate (100 mL), extracted with ethyl acetate (250 mL), dried over sodium sulphate and concentrated under reduced pressure. The crude product obtained upon evaporation of volatiles was purified by reverse phase column chromatography to obtain product 99-6 as a white solid 0.33 g (17%).

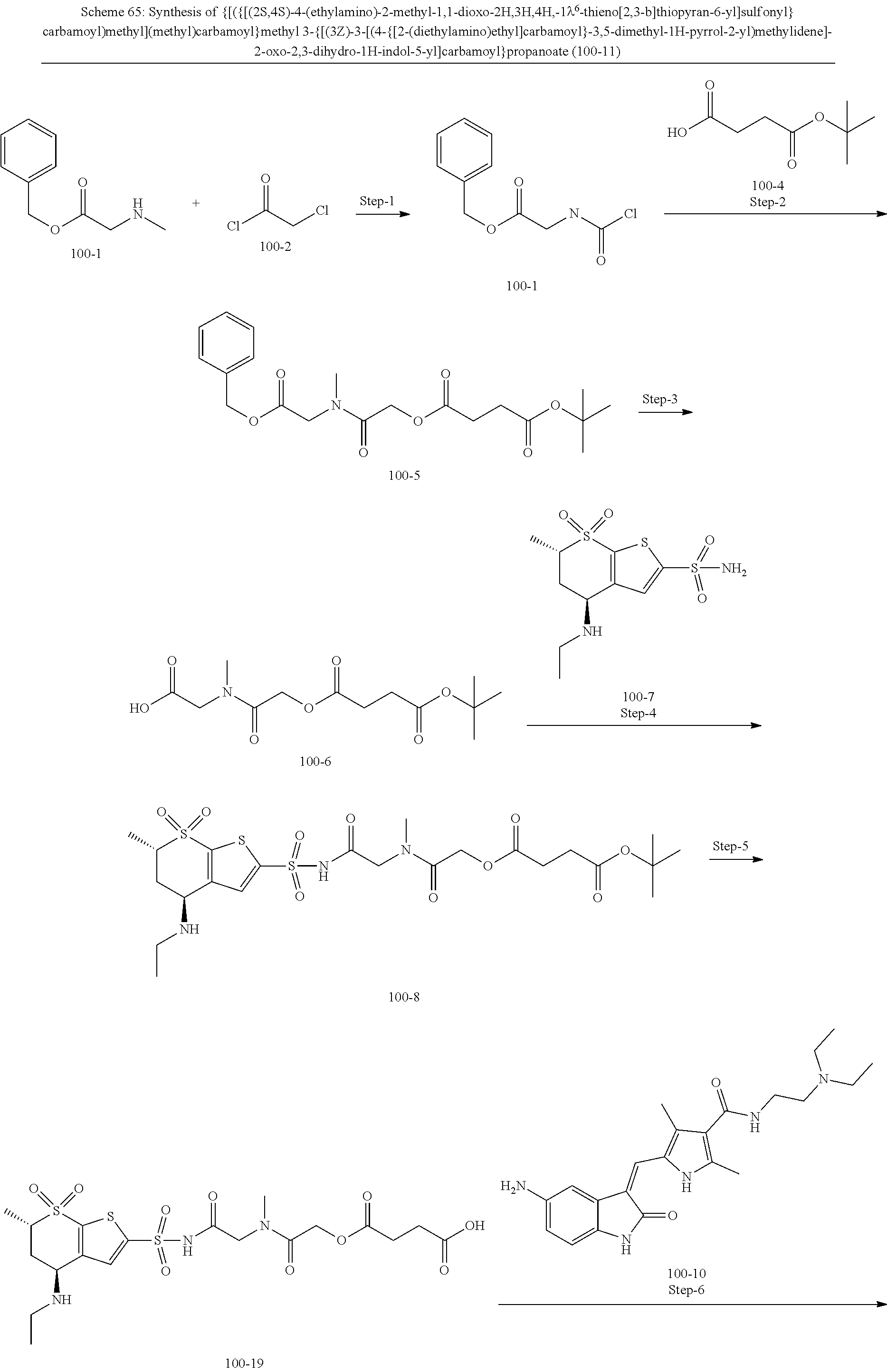

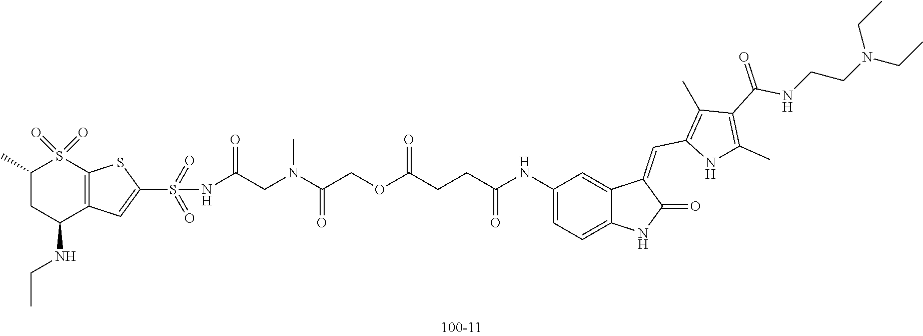 [SMILES: CCN[C@H]1C[C@H](C)S(=O)(=O)c2sc(S(=O)(=O)NC(=O)CN(C)C(=O)COC(=O)CCC(=O)Nc3ccc4c(c3)/C(=C/c3[nH]c(C)c(C(=O)NCCN(CC)CC)c3C)C(=O)N4)cc21]

Step-1: Preparation of benzyl [(chloroacetyl)(methyl)amino]acetate (100-3): To a solution of benzyl (methylamino)acetate 100-1 (10.0 g, 60.54 mmol) in dichloromethane (10 V) were added triethylamine (16.5 mL, 121.08 mmol), N,N-dimethylaminopyridine (0.738 g, 6.05 mmol) and chloroacetyl chloride 100-2 (6.25 mL, 78.7 mmol) at 0° C. The reaction mixture was allowed to stir at 25-30° C. over a period of 1 h. The resulting reaction mass was quenched with water (300 mL), extracted with ethyl acetate (500 mL×2), dried over sodium sulfate and concentrated under reduced pressure. The crude product obtained upon evaporation of volatiles was purified through silica gel (230-400 mesh) column chromatography (20-30% Ethyl acetate in hexanes) to obtain product 100-3 as an off-white solid 9.0 g (61.6%).

Step-2: Preparation of 1-{[2-(benzyloxy)-2-oxoethyl](methyl)carbamoyl}methyl 4-tert-butyl butanedioate (100-5): To a solution of 100-3 (1.8 g, 7.05 mmol) in N,N-dimethylformamide (5 V) were added sodium iodide (1.05 g, 7.05 mmol), 4-tert-butoxy-4-oxobutanoic acid 100-4 (1.22 g, 7.05 mmol), and triethylamine (1.98 mL, 14.11 mmol), at 25-30° C. The reaction mixture was allowed to stir at 55° C. over a period of 4 h. The resulting reaction mass was diluted with ethyl acetate (200 mL) and washed with water (100 mL×2), dried over sodium sulfate and concentrated under reduced pressure. The crude product obtained upon evaporation of volatiles was purified through silica gel (230-400 mesh) column chromatography (20-25% ethyl acetate in hexanes) to obtain product 100-5 as a colorless wax 1.1 g (40%).

Step-3: Preparation of 2-(2-{[4-(tert-butoxy)-4-oxobutanoyl]oxy}-N-methylacetamido)acetic acid (100-6): To a 250 mL Parr shaker vessel were added a solution 100-5 (1.1 g, 2.79 mmol) in ethyl acetate (10 V) and 10% Pd/C (0.11 g, 50% wet) at 25-30° C. The reaction mixture was stirred at 25-30° C. under hydrogen pressure (5 kg/cm²) over a period of 1 h. After completion of the reaction, the resulting reaction mixture was filtered through a celite bed and concentrated under reduced pressure to obtain product 100-6 as a waxy solid 0.8 g (94%).

Step-4: Preparation of 1-tert-butyl 4-{[({[(2S,4S)-4-(ethylamino)-2-methyl-1,1-dioxo-2H,3H,4H-1λ⁶-thieno[2,3-b]thiopyran-6-yl]sulfonyl}carbamoyl)methyl](methyl)carbamoyl}methyl butanedioate (100-8): To a solution of dorzolamide 100-7 (0.8 g, 2.22 mmol) in dichloromethane (10 V) were added N,N-diisopropylethylamine (0.80 mL, 4.45 mmol), EDC.HCl (0.63 g, 3.34 mmol), 2-(2-{[4-(tert-butoxy)-4-oxobutanoyl]oxy}-N-methylacetamido)acetic acid 100-6 (0.87 g, 2.89 mmol) and 4-dimethylaminopyridine (27 mg, 0.22 mmol) at 0° C. The reaction mixture was allowed to stir at 25-30° C. for 4 h. The resulting reaction mass was diluted with dichloromethane (300 mL), washed with water (100 mL), dried over sodium sulfate and concentrated under reduced pressure. The crude product obtained upon evaporation of volatiles was purified by silica gel (230-400 mesh) column chromatography to obtain product 100-8 as an off-white solid 1.0 g (74%).

Step-5: Preparation of 4-({[({[(2S,4S)-4-(ethylamino)-2-methyl-1,1-dioxo-2H,3H,4H-1λ⁶-thieno[2,3-b]thiopyran-6-yl]sulfonyl}carbamoyl)methyl](methyl)carbamoyl}methoxy)-4-oxobutanoic acid (100-9): To a solution of 1-tert-butyl 4-{[({[(2S,4S)-4-(ethylamino)-2-methyl-1,1-dioxo-2H,3H,4H-1λ⁶-thieno[2,3-b]thiopyran-6-yl]sulfonyl}carbamoyl)methyl](methyl)carbamoyl}methyl butanedioate 100-8 (1.1 g, 1.8 mmol) in dichloromethane (10 V) was added trifluoroacetic acid (3.3 mL, 3 V) slowly at 0° C. The reaction mixture was allowed to stir at 25-30° C. over a period of 1 h. After completion of the reaction, the resulting reaction mixture was concentrated under reduced pressure to obtain compound 100-9 as a TFA salt (colorless liquid, 0.8 g, 66%). The crude product 100-9 was taken forward to the next step without any further purification.

Step-6: Preparation of {[({[(2S,4S)-4-(ethylamino)-2-methyl-1,1-dioxo-2H,3H,4H-1λ⁶-thieno[2,3-b]thiopyran-6-yl]sulfonyl}carbamoyl)methyl](methyl)carbamoyl}methyl 3-{[(3Z)-3-[(4-{[2-(diethylamino)ethyl-]carbamoyl}-3,5-dimethyl-1H-pyrrol-2-yl)methylidene]-2-oxo-2,3-dihydro-1H-indol-5-yl]carbamoyl}propanoate (100-11): To a solution of 4-({[({[(2S,4S)-4-(ethylamino)-2-methyl-1,1-dioxo-2H,3H,4H-1λ⁶-thieno[2,3-b]thiopyran-6-yl]sulfonyl}carbamoyl)methyl](methyl)carbamoyl}methoxy)-4-oxobutanoic acid 100-9 (1.01 g, 1.51 mmol) in dichloromethane (10 V) were added NMM (0.34 mL, 3.16 mmol), EDC.HCl (0.29 g, 1.51 mmol), 4-dimethylaminopyridine (15 mg, 0.12 mmol) and 100-10 (0.5 g, 1.26 mmol) at 0° C. The reaction mixture was allowed to stir at 25-30° C. over a period of 16 h. The resulting reaction mass was diluted with dichloromethane (300 mL) and washed with water (100 mL), dried over sodium sulfate and concentrated under reduced pressure. The crude product obtained upon evaporation of volatiles was purified by reverse phase column chromatography to obtain product 100-11 as an orange solid 0.5 g (42%).

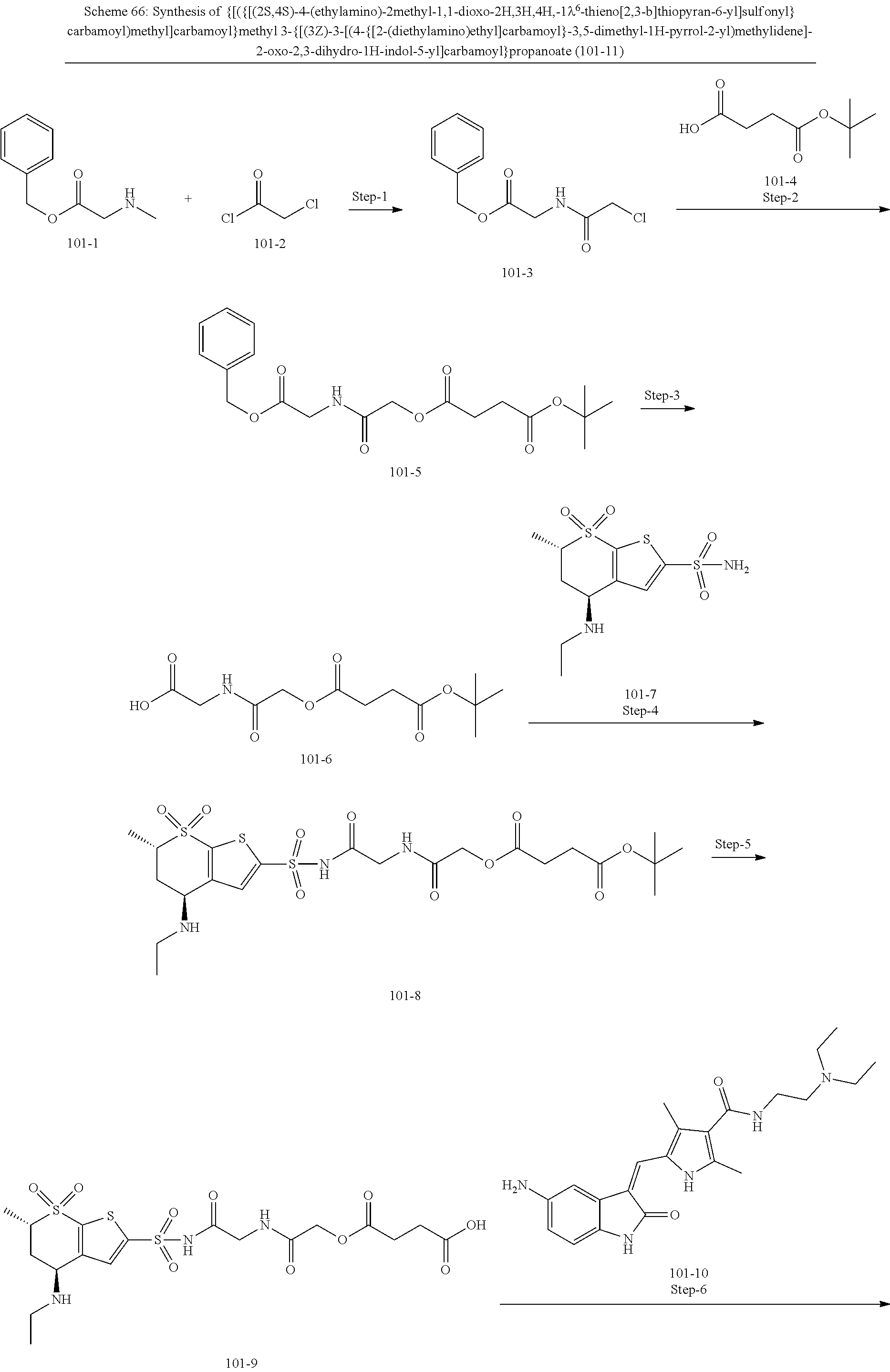

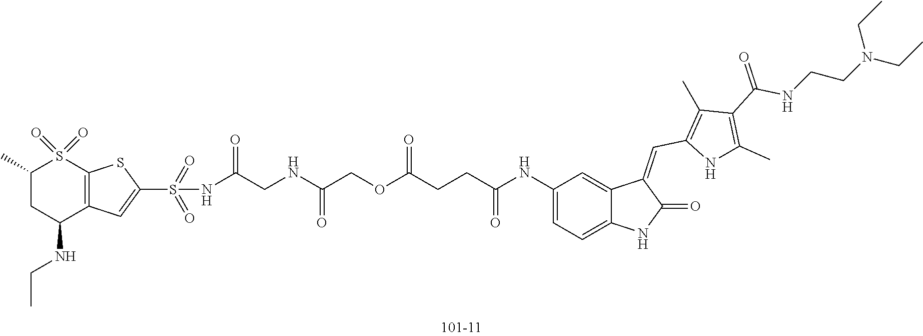 [SMILES: CCN[C@H]1C[C@H](C)S(=O)(=O)c2sc(S(=O)(=O)NC(=O)CNC(=O)COC(=O)CCC(=O)Nc3ccc4c(c3)/C(=C/c3[nH]c(C)c(C(=O)NCCN(CC)CC)c3C)C(=O)N4)cc21]

Step-1: Preparation of benzyl (2-chloroacetamido)acetate (101-3): To a solution of benzyl aminoacetate 101-1 (12 g, 72 mmol) in dichloromethane (10 V) were added triethylamine (26.2 mL, 181 mmol), N,N-dimethylaminopyridine (0.87 g, 7.0 mmol), chloroacetyl chloride (7 mL, 87 mmol) at 0° C. The reaction mixture was allowed to stir at 25-30° C. over a period of 1 h. The resulting reaction mass was quenched with water (250 mL), extracted with ethyl acetate (500 mL×2), dried over sodium sulfate and concentrated under reduced pressure. The crude product obtained upon evaporation of volatiles was purified by silica gel (230-400 mesh) column (25% Ethyl acetate in hexanes) to obtain product 101-3 as an off-white solid 5.0 g (41%).

Step-2: Preparation of 1-{[2-(benzyloxy)-2-oxoethyl]carbamoyl}methyl 4-tert-butyl butanedioate (101-5): To a solution of 101-3 (2.7 g, 11.2 mmol) in N,N-dimethylformamide (5 V) were added triethylamine (3.14 mL, 22.4 mmol), sodium iodide (2.33 g, 15.68 mmol) and 101-4 (2.53 g, 14.56 mmol) at 25-30° C. The reaction mixture was allowed to stir at 55° C. over a period of 4 h. The resulting reaction mass was diluted with ethyl acetate (500 mL) and washed with water (200 mL×2), dried over sodium sulfate and concentrated under reduced pressure. The crude product obtained upon evaporation of volatiles was purified through silica gel (230-400 mesh) column chromatography (50% ethyl acetate in hexanes) to obtain product 101-5 as a colorless wax 1.1 g (25%).

Step-3: Preparation of 2-(2-{[4-(tert-butoxy)-4-oxobutanoyl]oxy}acetamido)acetic acid (101-6): To a 250 mL Parr shaker vessel were added a solution 101-5 (1.1 g, 2.9 mmol) in ethyl acetate (10 V) and 10% Pd/C (0.11 g, 50% wet) at 25-30° C. The reaction mixture was stirred at 25-30° C. under hydrogen pressure (5 kg/cm²) over a period of 1 h. After completion of the reaction, the resulting reaction mixture was filtered through a celite bed and concentrated under reduced pressure to obtain product 101-6 as a waxy solid 0.76 g (91%).

Step-4: Preparation of 1-tert-butyl 4-{[({[(2S,4S)-4-(ethylamino)-2-methyl-1,1-dioxo-2H,3H,4H-1λ⁶-thieno[2,3-b]thiopyran-6-yl]sulfonyl}carbamoyl)methyl]carbamoyl}methyl butanedioate (101-8): To a solution of dorzolamide 101-7 (0.7 g, 1.94 mmol) in dichloromethane (10 V) were added N,N-diisopropylethylamine (0.88 mL, 4.87 mmol), EDC.HCl (0.67 g, 3.5 mmol), 101-6 (0.85 g, 2.92 mmol) and 4-dimethylamino pyridine (23 mg, 0.19 mmol) at 0° C. Reaction mixture was allowed to stir at 25-30° C. for 4 h. The resulting reaction mass was diluted with dichloromethane (300 mL) and washed with water (100 mL), dried over sodium sulfate and concentrated under reduced pressure. The crude product obtained upon evaporation of volatiles was purified by reverse phase column chromatography to obtain product 101-8 as an off-white solid 0.58 g (50%).

Step-5: Preparation of 4-({[({[(2S,4S)-4-(ethylamino)-2-methyl-1,1-dioxo-2H,3H,4H-1λ⁶-thieno[2,3-b]thiopyran-6-yl]sulfonyl}carbamoyl)methyl]carbamoyl}methoxy)-4-oxobutanoic acid (101-9): To a solution of 101-8 (0.58 g, 0.97 mmol) in dichloromethane (10 V) was added trifluoroacetic acid (3 V) slowly at 0° C. The reaction mixture was allowed to stir at 25-30° C. over a period of 1 h. After completion of the reaction, the resulting reaction mixture was concentrated under reduced pressure to obtain compound 101-9 as a TFA salt (pale brown wax, 0.65 g, 52%). The crude product 101-9 was taken forward to the next step without any further purification.

Step-6: Preparation of {[({[(2S,4S)-4-(ethylamino)-2-methyl-1,1-dioxo-2H,3H,4H-1λ⁶-thieno[2,3-b]thiopyran-6-yl]sulfonyl}carbamoyl)methyl]carbamoyl}methyl 3-{[(3Z)-3-[(4-{[2-(diethylamino)ethyl]carbamoyl}-3,5-dimethyl-1H-pyrrol-2-yl)methylidene]-2-oxo-2,3-dihydro-1H-indol-5-yl]carbamoyl}propanoate (101-11): To a solution of 101-9 (0.65 g, 1.2 mmol) in dichloromethane (10 V) were added NMM (0.27 mL, 2.5 mmol), EDC.HCl (0.23 g, 1.2 mmol), 4-dimethylaminopyridine (12 mg, 0.1 mmol) and 101-10 (0.4 g, 1.01 mmol) at 0° C. The reaction mixture was allowed to stir at 25-30° C. over a period of 16 h. The resulting reaction mass was diluted with dichloromethane (300 mL) and washed with water (100 mL), dried over sodium sulfate and concentrated under reduced pressure. The crude product obtained upon evaporation of volatiles was purified by reverse phase column chromatography to obtain product 101-11 as an orange solid 0.35 g (37%).

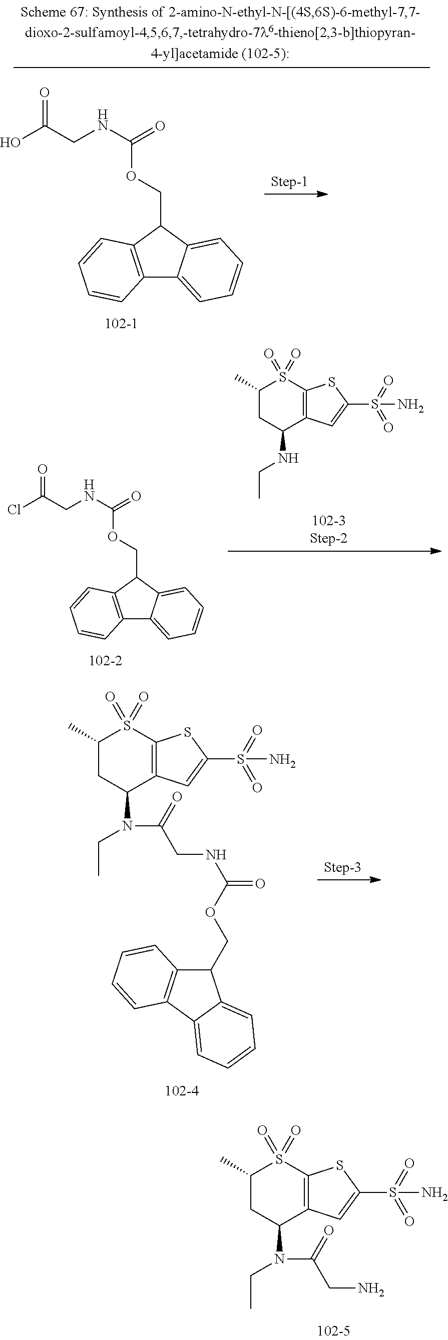 [SMILES: O=C(O)CNC(=O)OCC1c2ccccc2-c2ccccc21 ; O=C(Cl)CNC(=O)OCC1c2ccccc2-c2ccccc21 ; CCN[C@H]1C[C@H](C)S(=O)(=O)c2sc(S(N)(=O)=O)cc21 ; CCN(C(=O)CNC(=O)OCC1c2ccccc2-c2ccccc21)[C@H]1C[C@H](C)S(=O)(=O)c2sc(S(N)(=O)=O)cc21 ; CCN(C(=O)CN)[C@H]1C[C@H](C)S(=O)(=O)c2sc(S(N)(=O)=O)cc21]

Step-1: Preparation of (9H-fluoren-9-yl)methyl (2-chloro-2-oxoethyl)carbamate (102-2): To a solution of ({[(9H-fluoren-9-yl)methoxy]carbonyl}amino)acetic acid 102-1 (10.0 g, 6.71 mmol) in dichloromethane (8 V) and tetrahydrofuran (2.0 V) was added thionylchloride (1.94 mL, 26.8 mmol) at 0° C. The reaction was heated to 75° C. for 2 h. The resulting reaction mass was cooled to ambient temperature, diluted with ethyl acetate (500 mL), washed with water (250 mL×2), organic layer was dried over sodium sulfate and concentrated under reduced pressure to obtain compound 2 as an off white solid 6.0 g (47%). The crude compound 102-2 was taken forward to next step without any purification.

Step-2: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate (102-4): To a solution of dorzolamide 102-3 (1.0 g, 2.77 mmol) in dichloromethane (10 V) was added N,N-Diisopropylethylamine (1.0 mL, 5.5 mmol) at 0° C. After 30 min, 9H-fluoren-9-yl)methyl (2-chloro-2-oxoethyl)carbamate 102-2 (1.31 g, 4.1 mmol) was added at 0° C. The reaction mixture was allowed to stir at 25-30° C. over a period of 1 h. The resulting reaction mass was diluted with ethyl acetate (150 mL) and washed with water (50 mL×2), organic layer was dried over sodium sulfate and concentrated under reduced pressure to obtain compound 102-4 as an off white solid 0.67 g (40%). The crude compound 4 was taken forward to next step without any purification.

Step-3: Preparation of 2-amino-N-ethyl-N-[(4S,6S)-6-methyl-7,7-dioxo-2-sulfamoyl-4,5,6,7-tetrahydro-7l6-thieno[2,3-b]thiopyran-4-yl]acetamide (102-5): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)carbamate 102-4 (0.3 g, 0.49 mmol) in dichloromethane (5 V) was added piperidine (0.30 mL, 2.48 mmol) at 0° C. The reaction mixture was allowed to stir at 25-30° C. over a period of 24 hours. The resulting reaction mixture was concentrated under reduced pressure. The residue obtained upon evaporation of volatiles was purified by reverse phase column chromatography to obtain product 102-5 as a low melting white solid 0.18 g (13%).

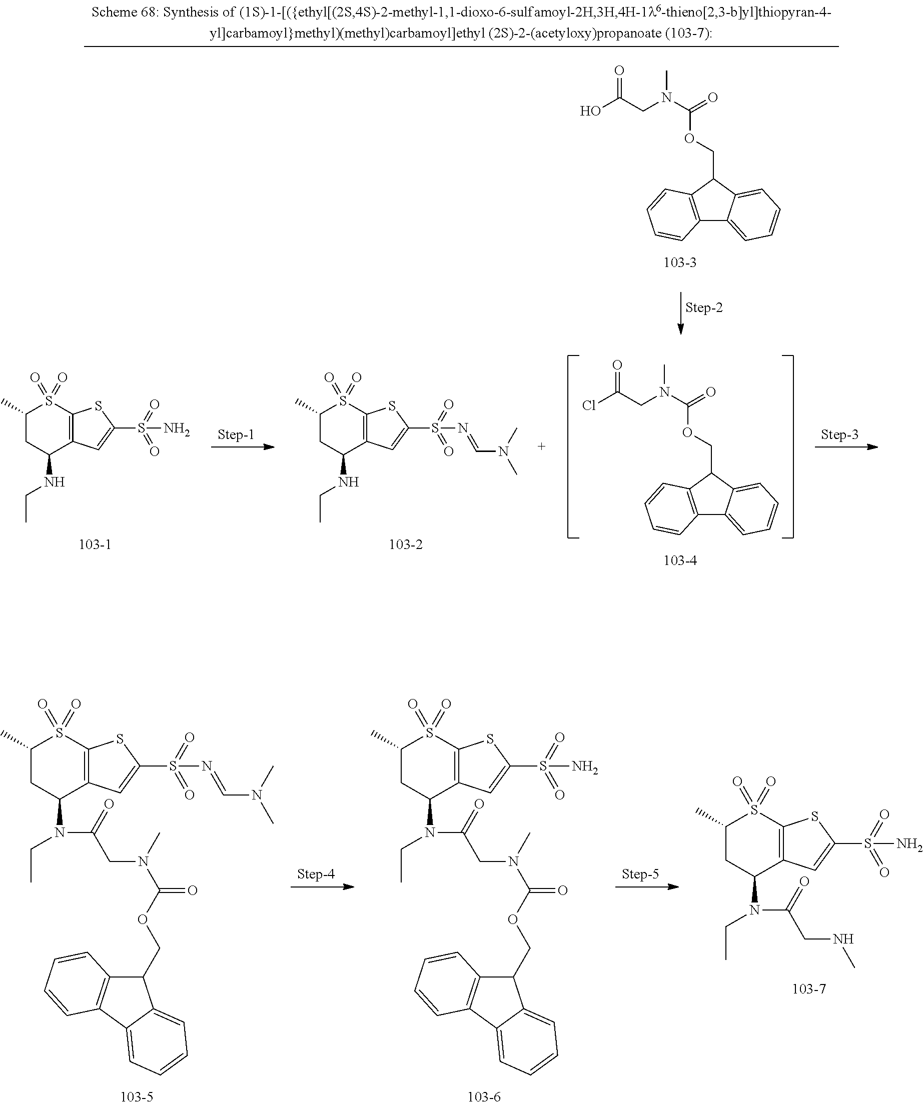

Step-1: Preparation of (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide (103-2): To a solution of (2S,4S)-4-(ethylamino)-2-methyl-1,1-dioxo-2H,3H,4H-1λ⁶-thieno[2,3-b]thiopyran-6-sulfonamide 103-1 (0.3 g, 0.83 mmol) in N,N-Dimethylformamide (0.6 mL), were added trimethylamine (0.12 mL, 0.91 mmol) and N,N-dimethylformamide dimethylacetal (0.13 mL, 0.99 mmol) at 0° C. The reaction mixture was allowed to stir at room temperature over a period of 16 hr. The resulting reaction mass was quenched with water (80 mL), extracted with dichloromethane (2×100 mL), organic layer was dried over sodium sulphate and concentrated under reduced pressure. The crude product obtained upon evaporation of volatiles was purified by silica gel column chromatography to obtain product 103-4 as a white solid 0.3 g (95%).

Step-2 & 3: Preparation of 9H-fluoren-9-ylmethyl N-({[(2S,4S)-6-{[(E)-[(dimethylamino)methylidene]amino]sulfonyl}-2-methyl-1,1-dioxo-2H,3H,4H-1λ⁶-thieno[2,3-b]thiopyran-4-yl](ethyl)carbamoyl}methyl)-N-methylcarbamate (103-5): To a solution of 2-{[(9H-fluoren-9-ylmethoxy)carbonyl](methyl)amino}acetic acid 103-3 (0.36 g, 1.18 mmol) in dichloromethane (20 mL), were added oxalyl chloride (0.29 mL, 3.4 mmol) and N,N-dimethylformamide (0.1 mL) at 0° C. The reaction mixture was allowed to stir at room temperature over a period of 30 min and concentrated to dryness under nitrogen atmosphere. The residue was diluted with dichloromethane (50 mL) and added N,N-diisopropylethylamine (0.28 mL, 1.5 mmol) followed by (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide 103-2 (0.3 g, 0.79 mmol) at 0° C. The reaction mixture was allowed to stir at room temperature over a period of 1 h. The resulting reaction mass was quenched with water (50 mL), extracted with dichloromethane (2×100 mL), organic layer was dried over sodium sulphate and concentrated under reduced pressure to obtain product 103-5 as colorless wax 0.45 g (87%). The crude compound as such taken into next step without any purification.

Step-4: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate (103-6): To a solution of 9H-fluoren-9-ylmethyl N-({[(2S,4S)-6-{[(E)-[(dimethylamino)methylidene]amino]sulfonyl}-2-methyl-1,1-dioxo-2H,3H,4H-1λ⁶-thieno[2,3-b]thiopyran-4-yl](ethyl)carbamoyl}methyl)-N-methylcarbamate 103-5 (2.5 g, 3.72 mmol) in methanol (10 mL) was added 50% aqueous HCl solution at room temperature. The reaction mixture was allowed to stir at 50° C. over a period of 12 h. Further the reaction mixture was allowed to stir at 100° C. over a period of 12 h. The resulting reaction mass was quenched with water (50 mL), extracted with ethyl acetate (100×2 mL), dried over sodium sulphate and concentrated under reduced pressure to obtain product 103-6 as an off white solid 2.1 g (95%).

Step-5: Preparation of 2-amino-N-ethyl-N-[(2S,4S)-2-methyl-1,1-dioxo-6-sulfamoyl-2H,3H,4H-1λ⁶-thieno[2,3-b]thiopyran-4-yl]acetamide (103-7): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate 103-6 (1.8 g, 2.91 mmol) in dichloromethane (5 V) was added piperidine (1.44 mL, 14.5 mmol) at 0° C. The reaction mixture was allowed to stir at 25-30° C. over a period of 4 hours. The resulting reaction mixture was concentrated under reduced pressure. The crude compound was purified by preparative HPLC to obtain product 103-7 as a low melting off white solid 1.0 g (86%).

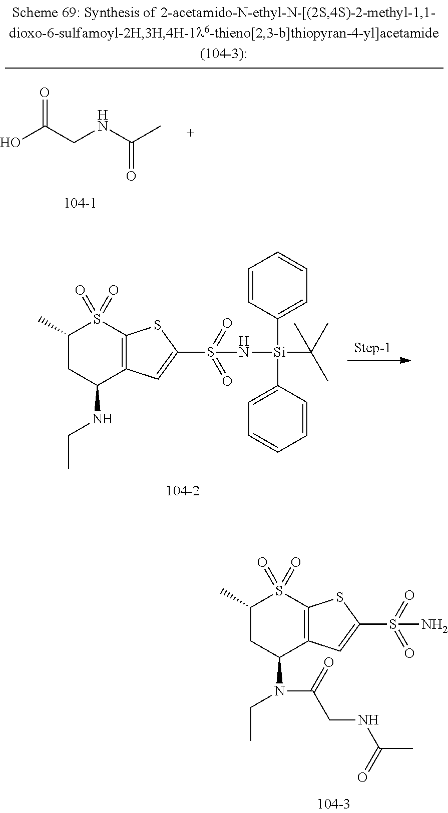

Step-1: Preparation of 2-acetamido-N-ethyl-N-[(2S,4S)-2-methyl-1,1-dioxo-6-sulfamoyl-2H,3H,4H-1λ⁶-thieno[2,3-b]thiopyran-4-yl]acetamide (104-3): To a solution of (2S,4S)-N-(tert-butyldiphenylsilyl)-4-(ethylamino)-2-methyl-1,1-dioxo-2H,3H,4H-1λ⁶-thieno[2,3-b]thiopyran-6-sulfonamide 104-2 (0.1 g, 0.18 mmol) in dichloromethane (20 mL), were added HATU (0.69 g, 0.18 mmol), DIPEA (0.05 mL, 0.27 mmol) and 2-acetamidoacetic acid (0.021 g, 0.18 mmol) at 0° C. The reaction mixture was allowed to stir at 25-30° C. temperature over a period of 2 h. The resulting reaction mass was quenched with water (25 mL), extracted with dichloromethane (75 mL×2), dried over sodium sulfate and concentrated under reduced pressure. The residue obtained upon evaporation of volatiles was purified by preparative HPLC to obtain product 104-3 as a low melting white solid 7 mg (9%).

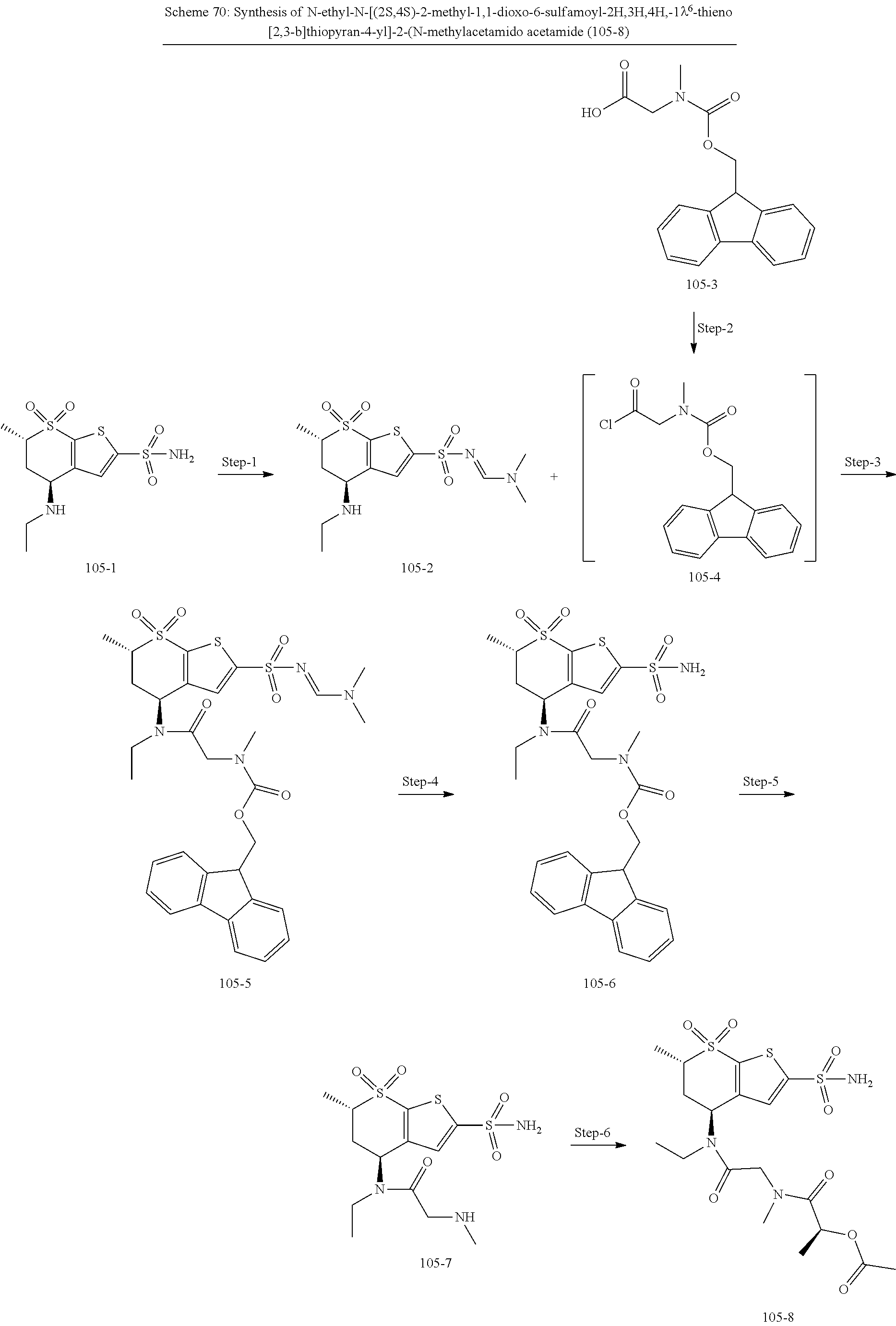

Step-1: Preparation of (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide (105-2): To a solution of (2S,4S)-4-(ethylamino)-2-methyl-1,1-dioxo-2H,3H,4H-1λ⁶-thieno[2,3-b]thiopyran-6-sulfonamide 105-1 (0.3 g, 0.83 mmol) in N,N-Dimethylformamide (0.6 mL), were added trimethylamine (0.12 mL, 0.91 mmol) and N,N-dimethylformamide dimethylacetal (0.13 mL, 0.99 mmol) at 0° C. The reaction mixture was allowed to stir at room temperature over a period of 16 hr. The resulting reaction mass was quenched with water (80 mL), extracted with dichloromethane (2×100 mL), organic layer was dried over sodium sulphate and concentrated under reduced pressure. The crude product obtained upon evaporation of volatiles was purified by silica gel column chromatography to obtain product 105-4 as a white solid 0.3 g (95%).

Step-2 & 3: Preparation of 9H-fluoren-9-ylmethyl N-({[(2S,4S)-6-{[(E)-[(dimethylamino)methylidene]amino]sulfonyl}-2-methyl-1,1-dioxo-2H,3H,4H-1λ⁶-thieno[2,3-b]thiopyran-4-yl](ethyl)carbamoyl}methyl)-N-methylcarbamate (105-5): To a solution of 2-{[(9H-fluoren-9-ylmethoxy)carbonyl](methyl)amino}acetic acid 105-3 (0.36 g, 1.18 mmol) in dichloromethane (20 mL), were added oxalyl chloride (0.29 mL, 3.4 mmol) and N,N-dimethylformamide (0.1 mL) at 0° C. The reaction mixture was allowed to stir at room temperature over a period of 30 min and concentrated to dryness under nitrogen atmosphere. The residue was diluted with dichloromethane (50 mL) and added N,N-diisopropylethylamine (0.28 mL, 1.5 mmol) followed by (E)-N′-{[(2S,4S)-4-(ethylamino)-2-methyl-1,1-dioxo-2H,3H,4H-1λ⁶-thieno[2,3-b]thiopyran-6-yl]sulfonyl}-N,N-dimethylmethanimidamide 105-2 (0.3 g, 0.79 mmol) at 0° C. The reaction mixture was allowed to stir at room temperature over a period of 1 h. The resulting reaction mass was quenched with water (50 mL), extracted with dichloromethane (2×100 mL), organic layer was dried over sodium sulphate and concentrated under reduced pressure to obtain product 105-5 as colorless wax 0.45 g (87%). The crude compound as such taken into next step without any purification.

Step-4: Preparation of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate (105-6): To a solution of 9H-fluoren-9-ylmethyl N-({[(2S,4S)-6-{[(E)-[(dimethylamino)methylidene]amino]sulfonyl}-2-methyl-1,1-dioxo-2H,3H,4H-1λ⁶-thieno[2,3-b]thiopyran-4-yl](ethyl)carbamoyl}methyl)-N-methylcarbamate 105-5 (2.5 g, 3.72 mmol) in methanol (10 mL) was added 50% aqueous HCl solution at room temperature. The reaction mixture was allowed to stir at 50° C. over a period of 12 h. Further the reaction mixture was allowed to stir at 100° C. over a period of 12 h. The resulting reaction mass was quenched with water (50 mL), extracted with ethyl acetate (100×2 mL), dried over sodium sulphate and concentrated under reduced pressure to obtain product 105-6 as an off white solid 2.1 g (95%).

Step-5: Preparation of 2-amino-N-ethyl-N-[(2S,4S)-2-methyl-1,1-dioxo-6-sulfamoyl-2H,3H,4H-1λ⁶-thieno[2,3-b]thiopyran-4-yl]acetamide (105-7): To a solution of 9H-fluoren-9-ylmethyl N-({ethyl[(2S,4S)-2-methyl-1,1-dioxo-6-sulfamoyl-2H,3H,4H-1λ⁶-thieno[2,3-b]thiopyran-4-yl]carbamoyl}methyl)-N-methylcarbamate 105-6 (1.8 g, 2.91 mmol) in dichloromethane (5 V) was added piperidine (1.44 mL, 14.5 mmol) at 0° C. The reaction mixture was allowed to stir at 25-30° C. over a period of 4 hours. The resulting reaction mixture was concentrated under reduced pressure. The crude compound was purified by preparative HPLC to obtain product 105-7 as a low melting off white solid 1.0 g (86%).

Step-6: Preparation of N-ethyl-N-[(2S,4S)-2-methyl-1,1-dioxo-6-sulfamoyl-2H,3H,4H-1λ⁶-thieno[2,3-b]thiopyran-4-yl]-2-(N-methylacetamido)acetamide (105-8): To a solution of N-ethyl-N-[(2S,4S)-2-methyl-1,1-dioxo-6-sulfamoyl-2H,3H,4H-1λ⁶-thieno[2,3-b]thiopyran-4-yl]-2-(methylamino)acetamide 105-7 (1.0 g, 2.53 mmol) in dichloromethane (20 mL), were added N,N-Diisopropylethylamine (0.69 mL, 3.79 mmol) and acetyl chloride (0.18 mL, 2.53 mmol) at 0° C. The reaction mixture was allowed to stir at 25-30° C. temperature over a period of 2 h. The resulting reaction mass was quenched with water (150 mL), extracted with dichloromethane (150 mL×2), dried over sodium sulfate and concentrated under reduced pressure. The crude compound was purified by preparative HPLC to obtain product 105-8 as an off white solid 0.5 g (47%).

This specification has been described with reference to embodiments of the invention. However, one of ordinary skill in the art appreciates that various modifications and changes can be made without departing from the scope of the invention as set forth herein. Accordingly, the specification is to be regarded in an illustrative rather than a restrictive sense, and all such modifications are intended to be included within the scope of invention.

What is claimed is:

1. A compound of Formula I, Formula II, Formula III, Formula IV, Formula V, Formula VI, or Formula VII: 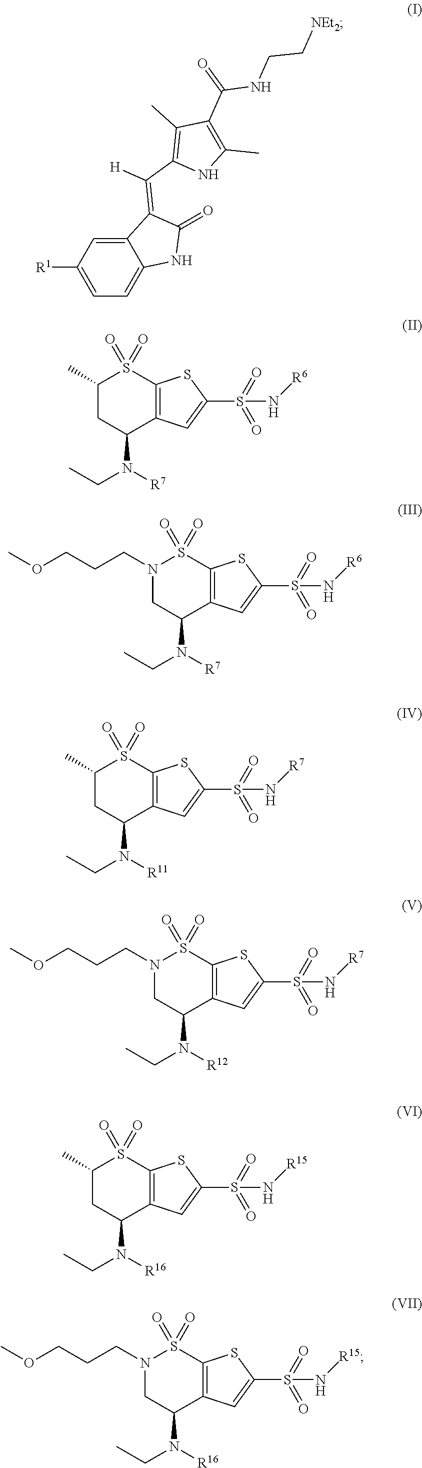
or a pharmaceutically acceptable salt thereof,
wherein:
R¹ is selected from 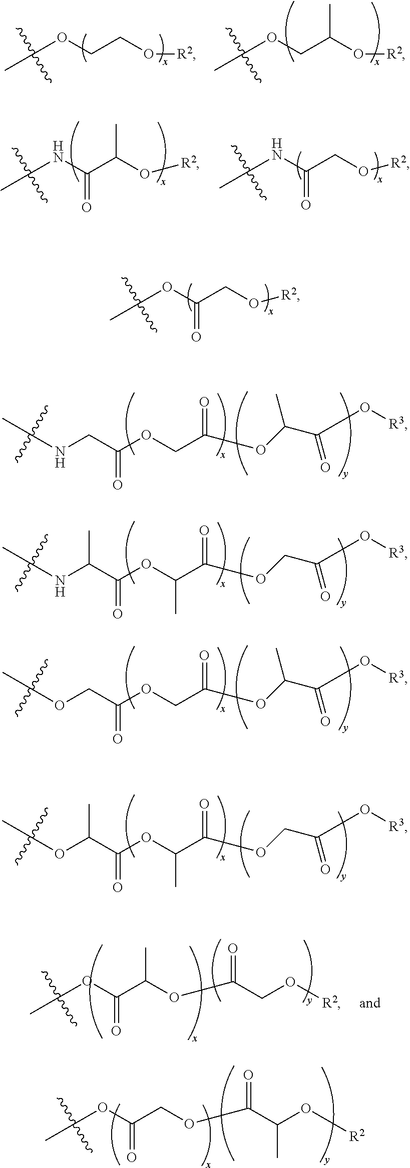
R² is selected from hydrogen, —CH₂COOH, —C(O)R⁴, alkenyl, alkynyl, cycloalkyl, cycloalkylalkyl, heterocyclyl, heterocycloalkyl, aryl, arylalkyl, heteroaryl, and heteroarylalkyl;
R³ is selected from hydrogen, alkenyl, alkynyl, cycloalkyl, cycloalkylalkyl, heterocyclyl, heterocycloalkyl, aryl, arylalkyl, heteroaryl, and heteroarylalkyl;
R⁴ is selected from hydrogen, alkyl, cycloalkyl, cycloalkylalkyl, heterocycle, heterocycloalkyl, aryl, arylalkyl, heteroaryl, and heteroarylalkyl; and
R⁶ is selected from 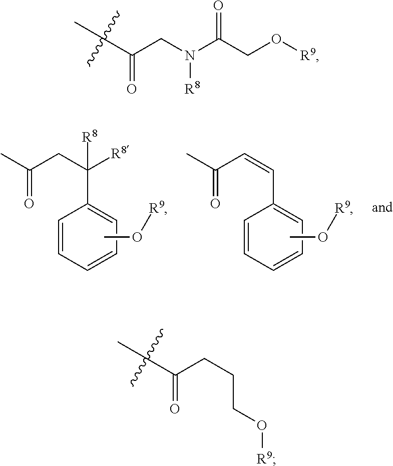
R⁷ is hydrogen or —C(O)R⁴;
R⁸ and R^{8′} are independently selected from hydrogen and C₁₋₆alkyl;
R⁹ is —C(O)R⁴, —C(O)CH₂OR⁴, 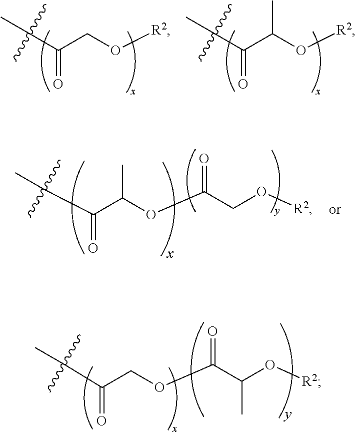
R¹¹ is selected from 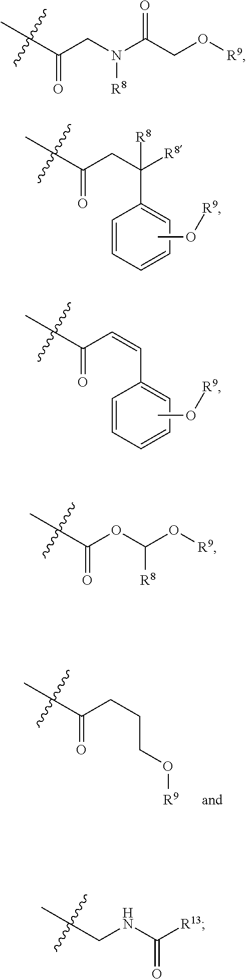
R¹² is selected from 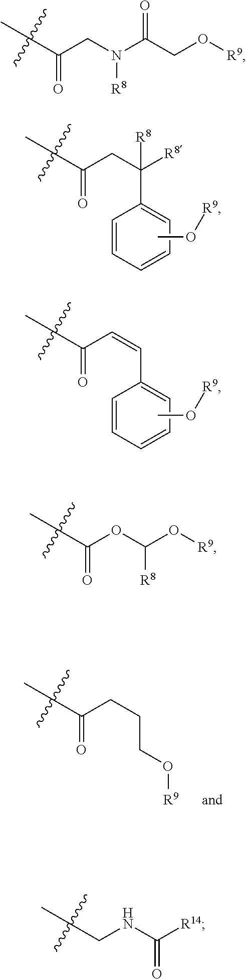
R¹³ is C₄₋₆alkyl, C₃₋₇cycloalkyl, cycloalkylalkyl, heterocycle, heterocycloalkyl, aryl, arylalkyl, heteroaryl, or heteroarylalkyl;
R¹⁴ is C₁₋₆alkyl, C₃₋₇cycloalkyl, cycloalkylalkyl, heterocycle, heterocycloalkyl, aryl, arylalkyl, heteroaryl, or heteroarylalkyl;
R¹⁵ is selected from —C(O)R⁴, 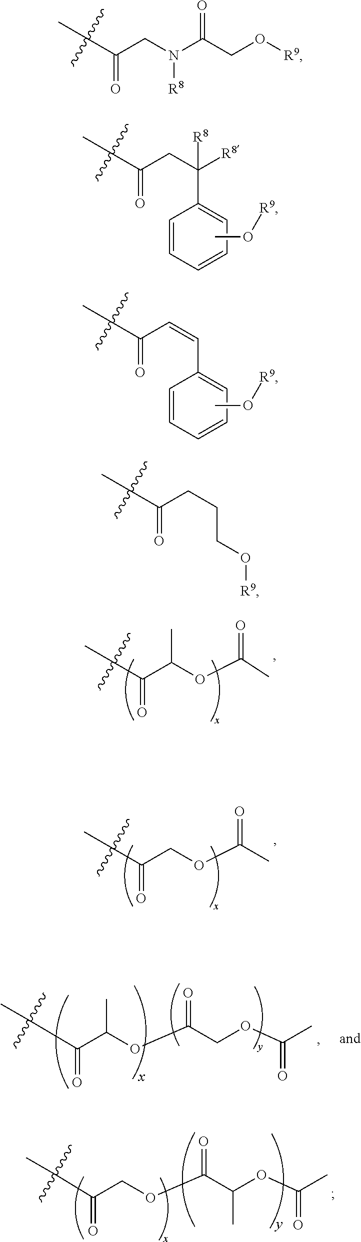
R¹⁶ is selected from 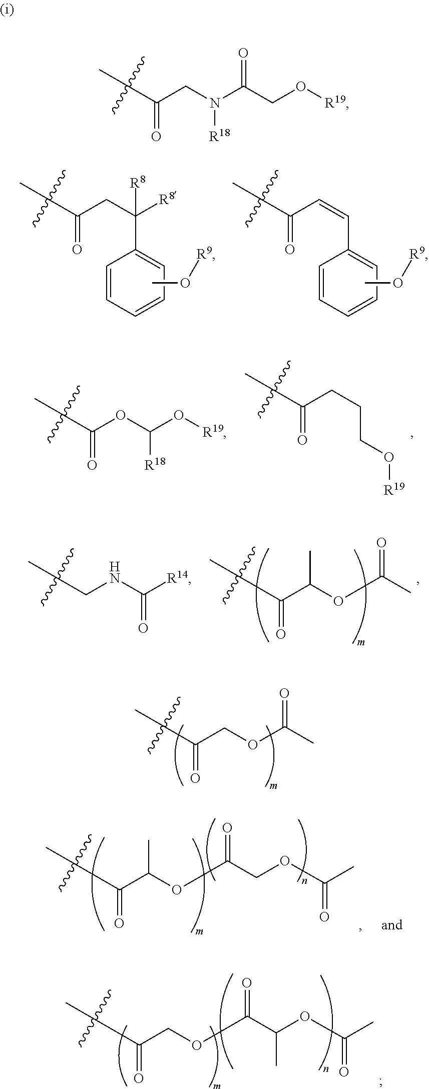
and
(ii) —C(O)R⁴ when R¹⁵ is —C(O)R⁴, 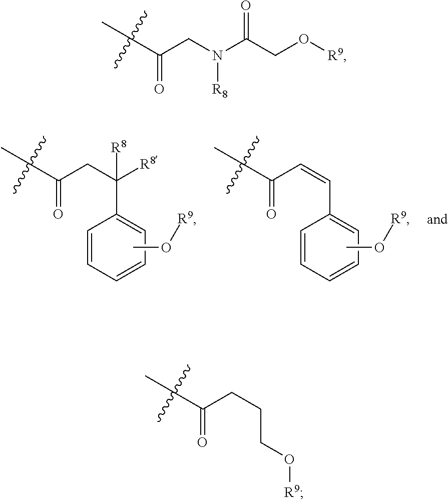
R¹⁸ is selected from hydrogen and C₁₋₆alkyl; and
R¹⁹ is —C(O)R⁴, C(O)CH₂OR⁴, 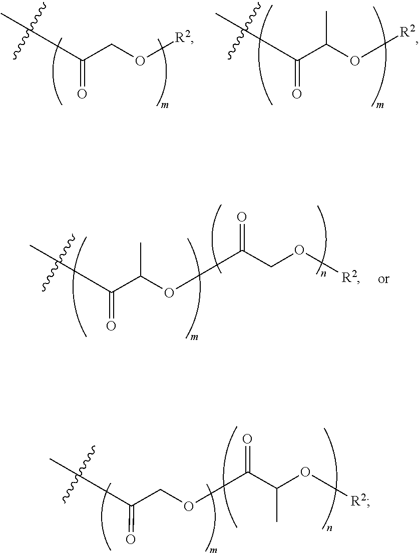
R² is selected from hydrogen, —CH₂COOH, —C(O)R⁴, alkenyl, alkynyl, cycloalkyl, cycloalkylalkyl, heterocyclyl, heterocycloalkyl, aryl, arylalkyl, heteroaryl, and heteroarylalkyl;
R⁴ is selected from hydrogen, alkyl, cycloalkyl, cycloalkylalkyl, heterocycle, heterocycloalkyl, aryl, arylalkyl, heteroaryl, and heteroarylalkyl;
x and y are an integer independently selected from 1, 2, 3, 4, 5, 6, 7, 8, 9, 10, 11, 12, 13, 14, 15, 16, 17, 18, 19, and 20; and
m and n are an integer independently selected from 1, 2, 3, 4, 5, 6, 7, 8, 9, 10, 11, 12, 13, 14, 15, 16, 17, 18, 19, and 20.

2. The compound of claim 1 of Formula II or Formula III:

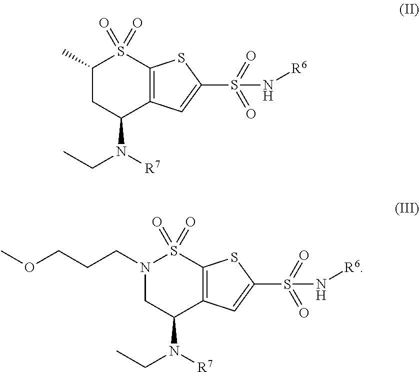

3. The compound of claim 1 of Formula IV or Formula V:

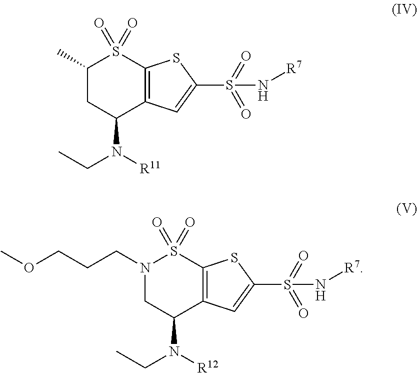

4. The compound of claim 1 of Formula VI or Formula VII:

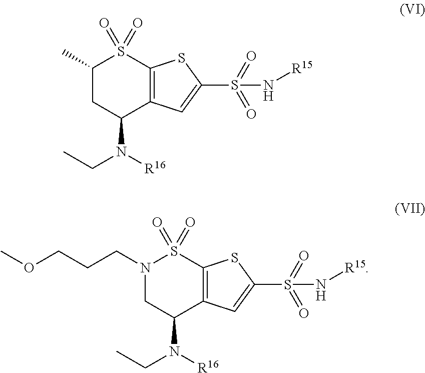

5. The compound of claim 2, wherein R⁶ is selected from

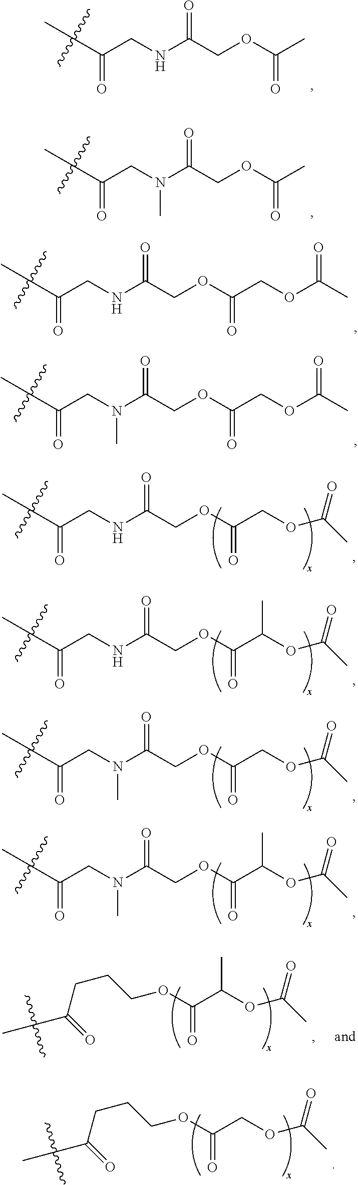

6. The compound of claim 2, wherein R⁶ is selected from

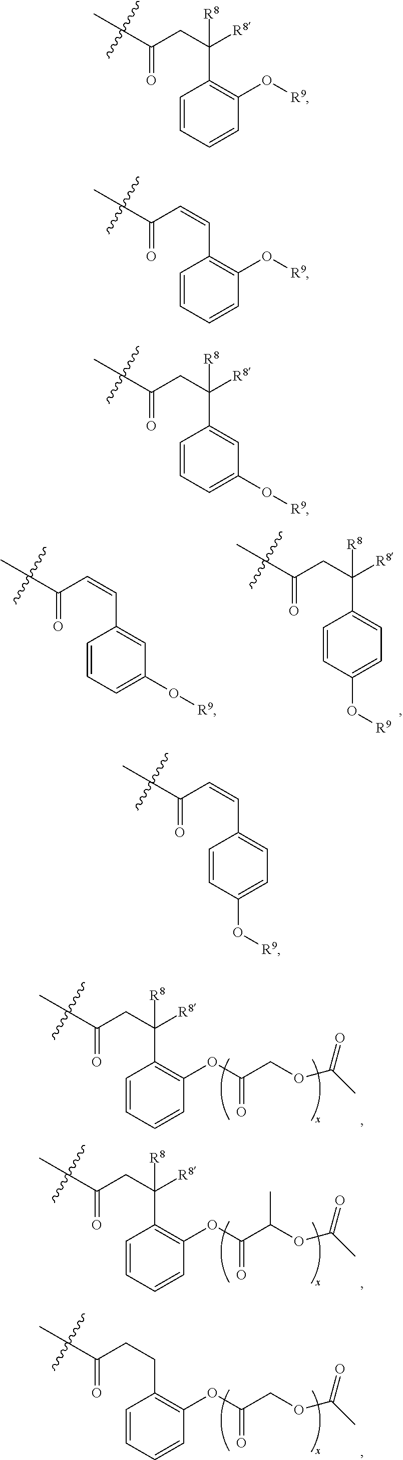

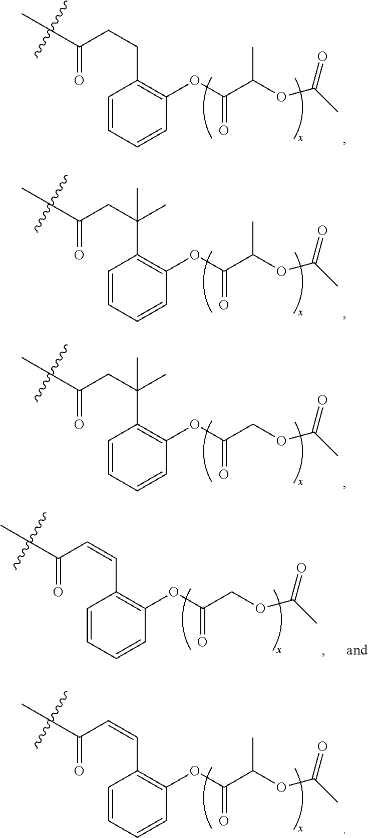

7. The compound of claim 3, wherein R¹¹ or R¹² is selected from

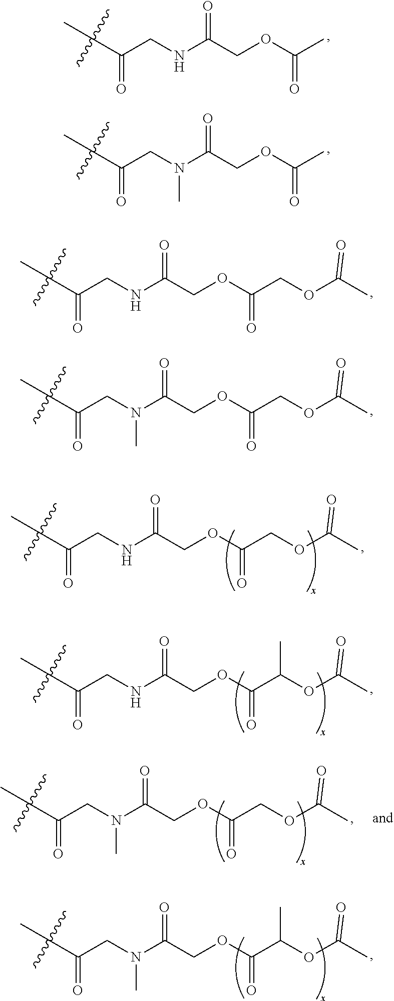

8. The compound of claim 3, wherein R¹¹ or R¹² is selected from

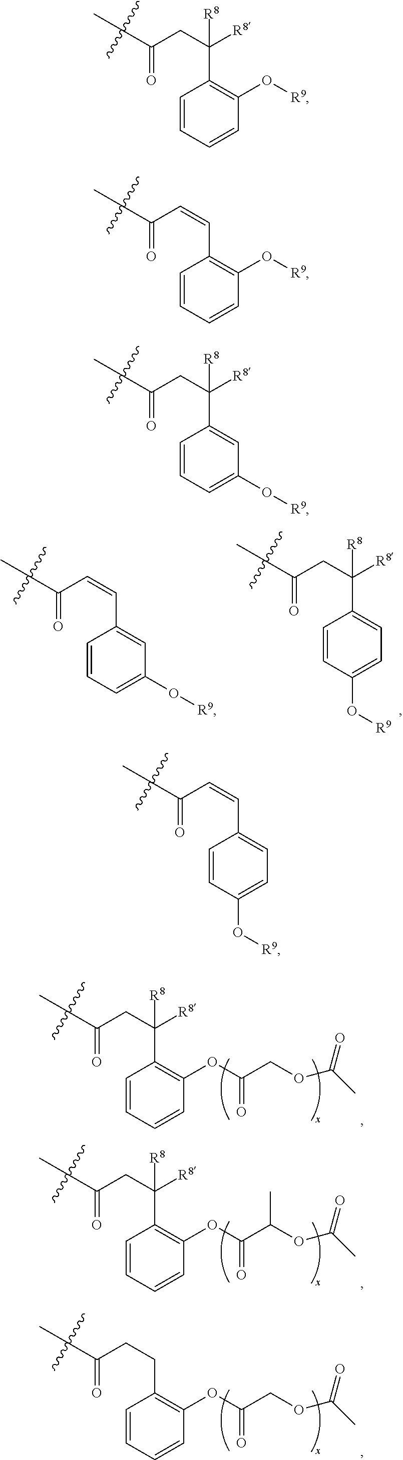

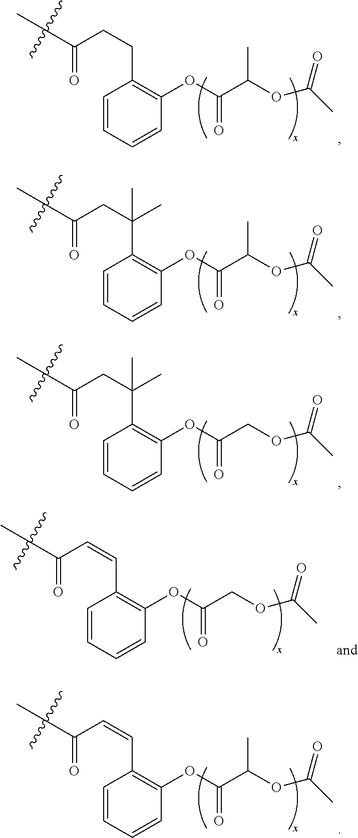

9. The compound of claim 3, wherein R¹¹ or R¹² is selected from

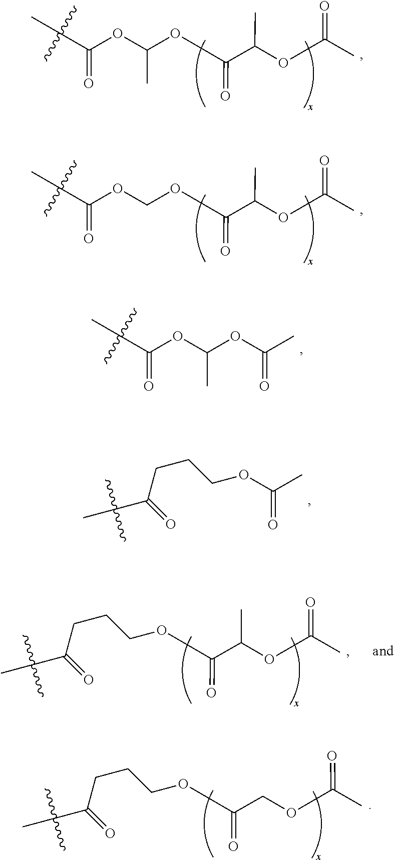

10. The compound of claim 1, wherein R⁷ is hydrogen.

11. The compound of claim 4, wherein R¹⁵ is selected from

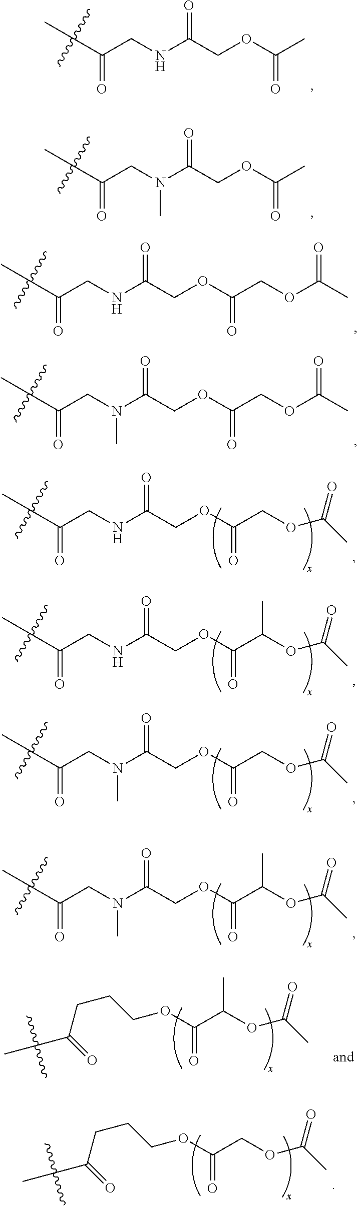

12. The compound of claim 4, wherein R¹⁵ is selected from

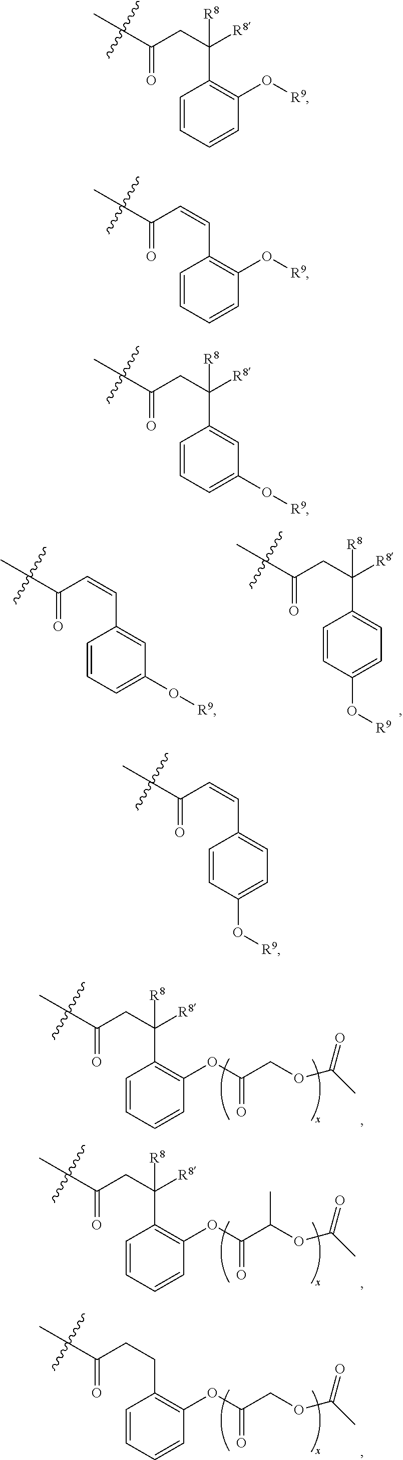

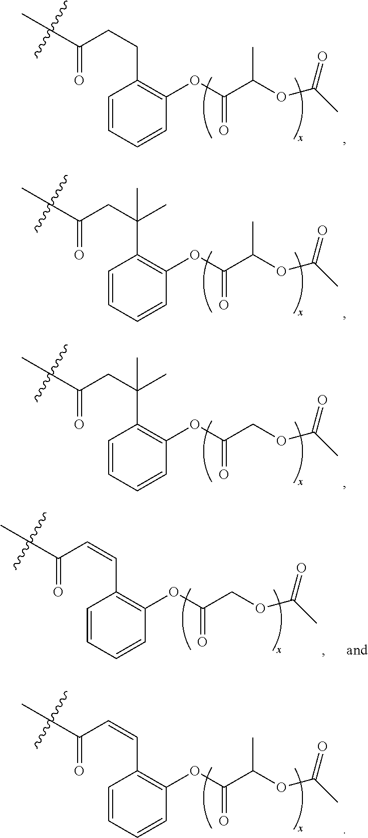

13. The compound of claim 4, wherein R¹⁶ is selected from

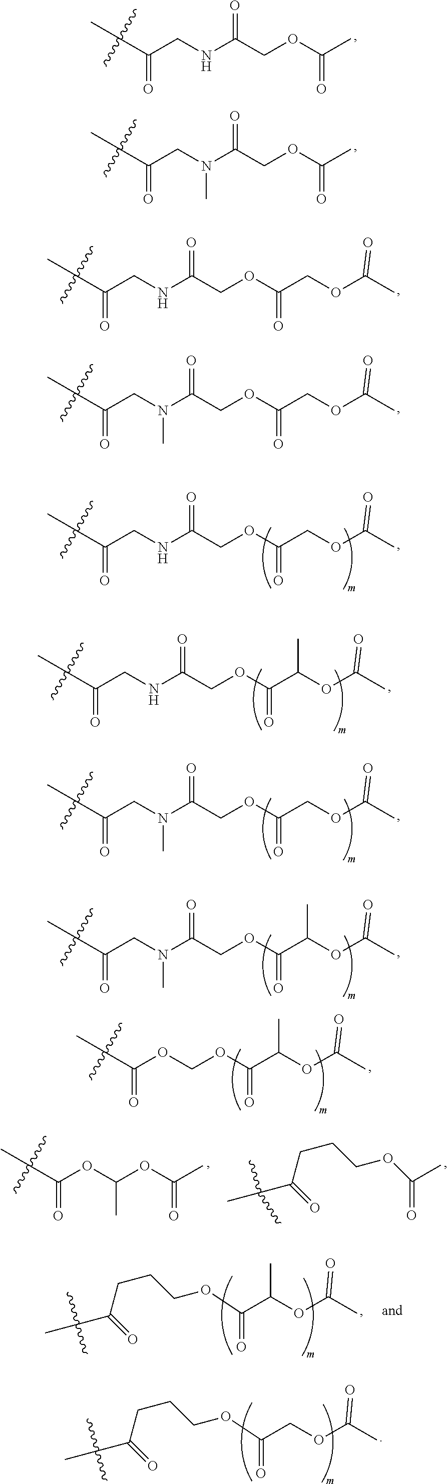

14. The compound of claim 4, wherein R¹⁶ is selected from

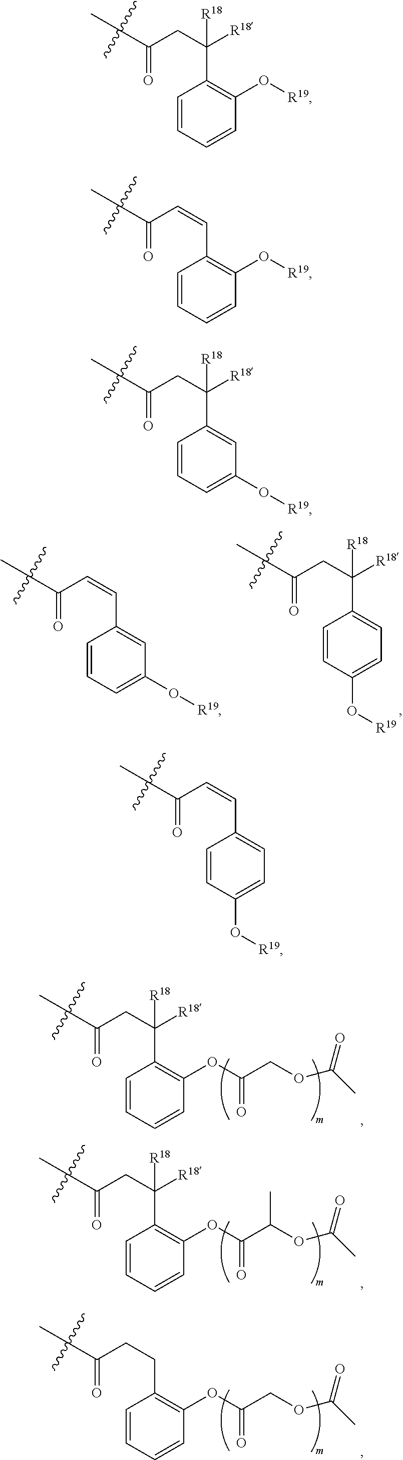

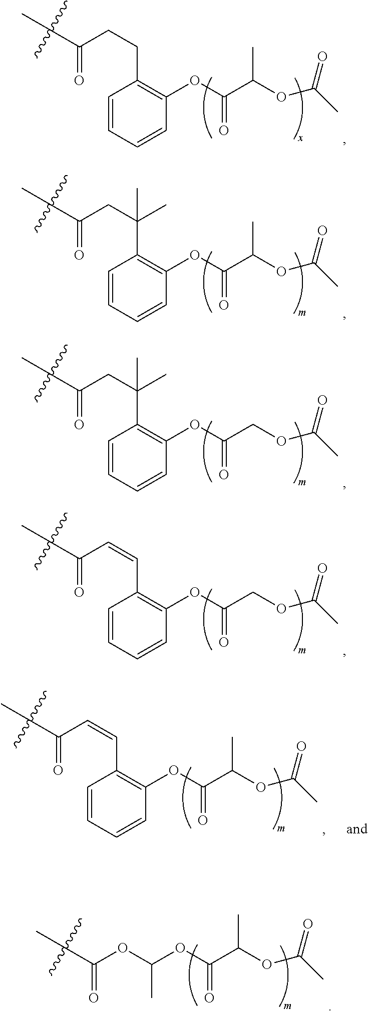

15. A compound of Formula VIII, Formula IX, Formula X, Formula XI, Formula XII, Formula XIII, Formula XIV, or Formula V: 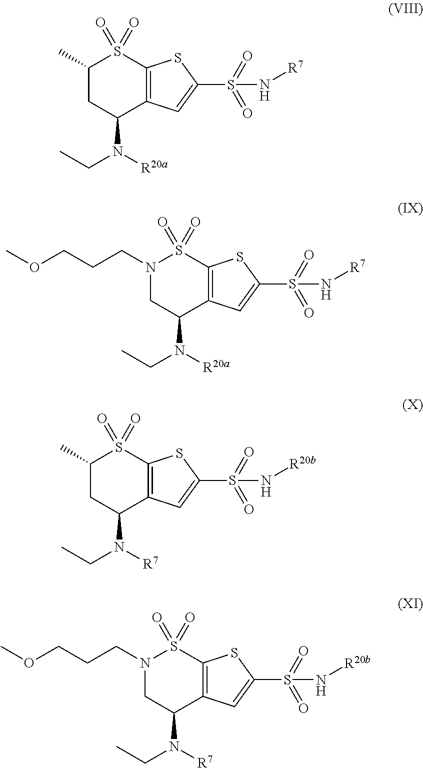 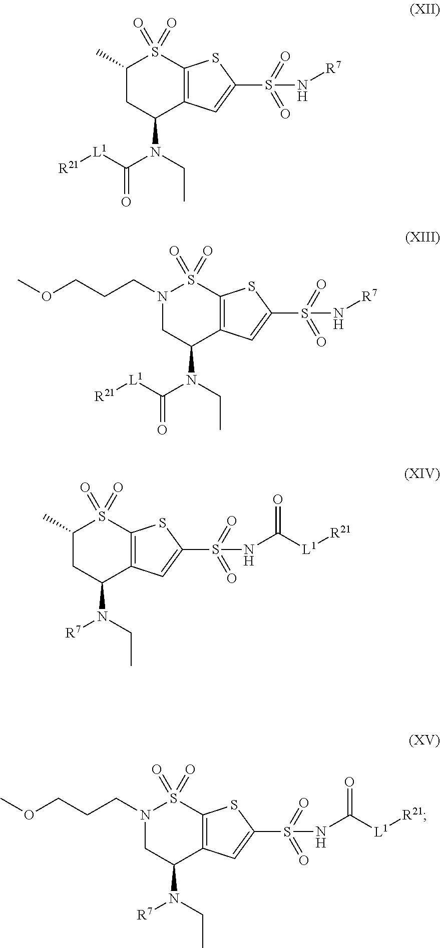
or a pharmaceutically acceptable salt thereof;
wherein
R² is selected from hydrogen, —CH₂COOH, —C(O)R⁴, alkenyl, alkynyl, cycloalkyl, cycloalkylalkyl, heterocyclyl, heterocycloalkyl, aryl, arylalkyl, heteroaryl, and heteroarylalkyl;
R⁴ is selected from hydrogen, alkyl, cycloalkyl, cycloalkylalkyl, heterocycle, heterocycloalkyl, aryl, arylalkyl, heteroaryl, and heteroarylalkyl;
R⁷ is hydrogen or —C(O)R⁴;
R⁸ and R^{8′} are independently selected from hydrogen and C₁₋₆alkyl;
R⁹ is —C(O)R⁴, —C(O)CH₂OR⁴, 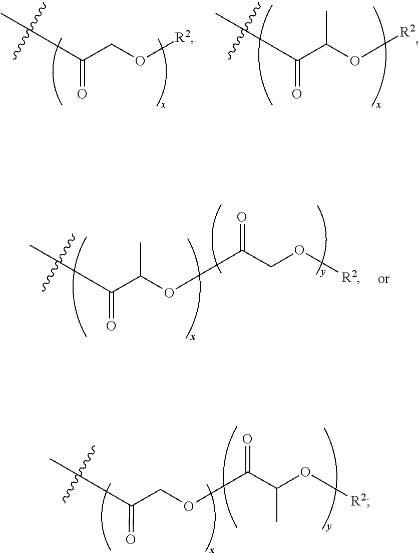
R^{20a} is selected from 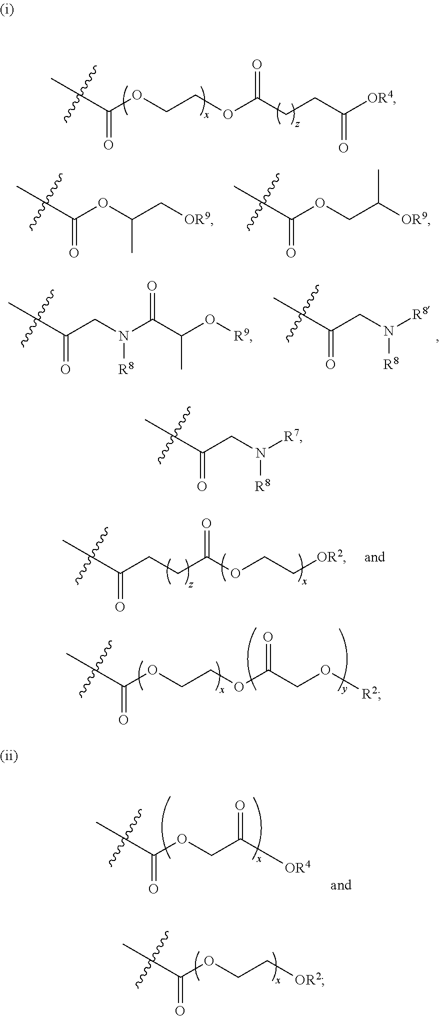
R^{20b} is selected from 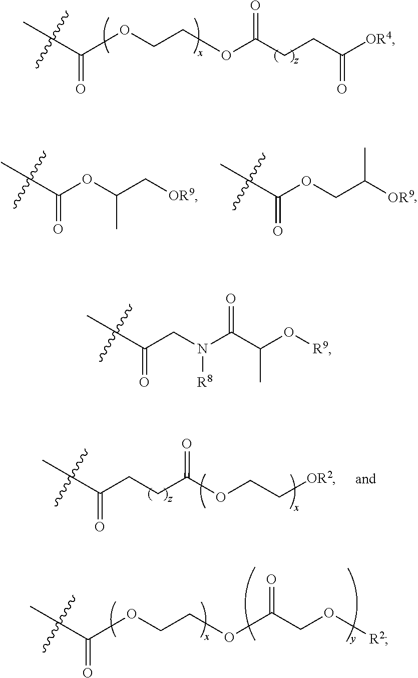
wherein R⁹ is not —C(O)R⁴ when R^{20b} is 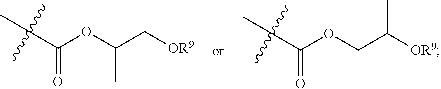
R²¹ is selected from 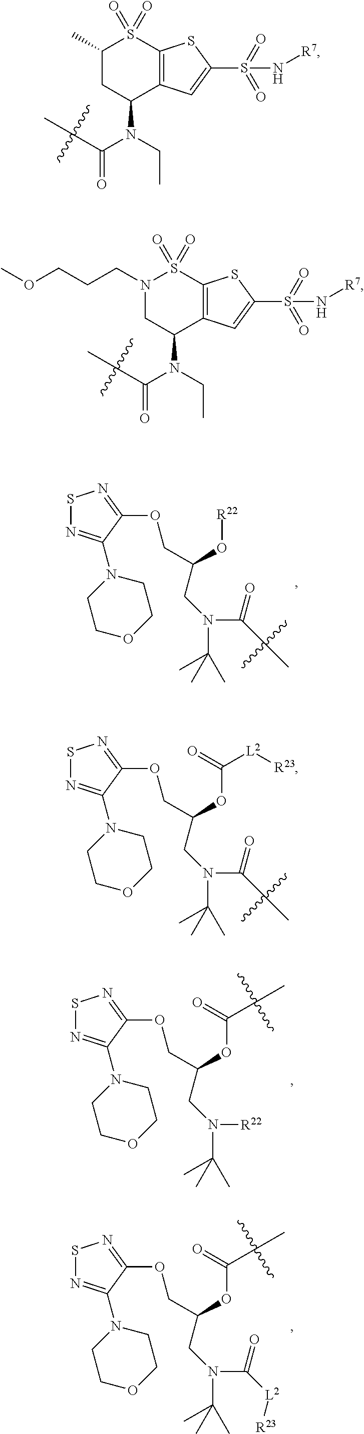 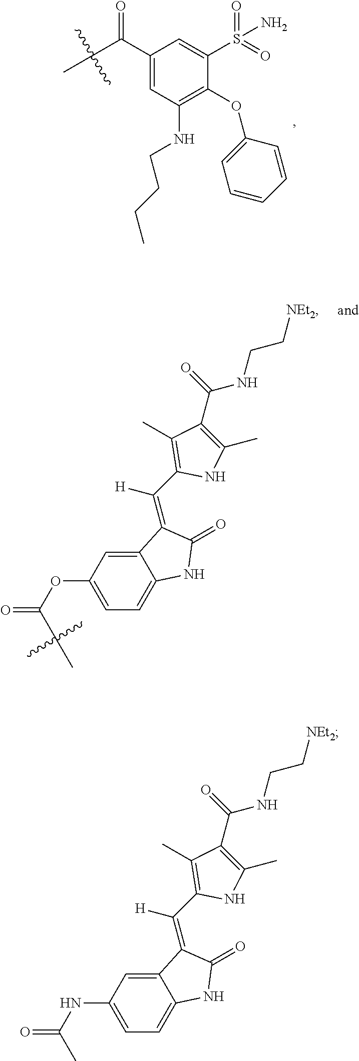
R²² is selected from 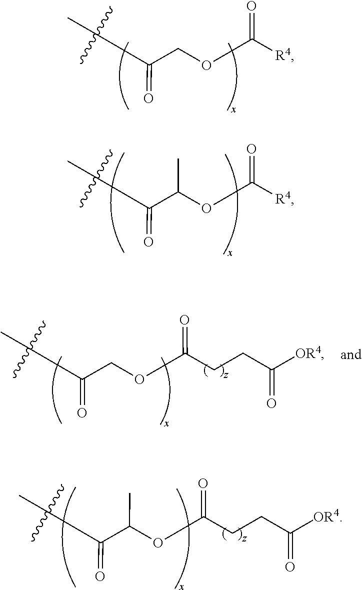
R²³ is selected from 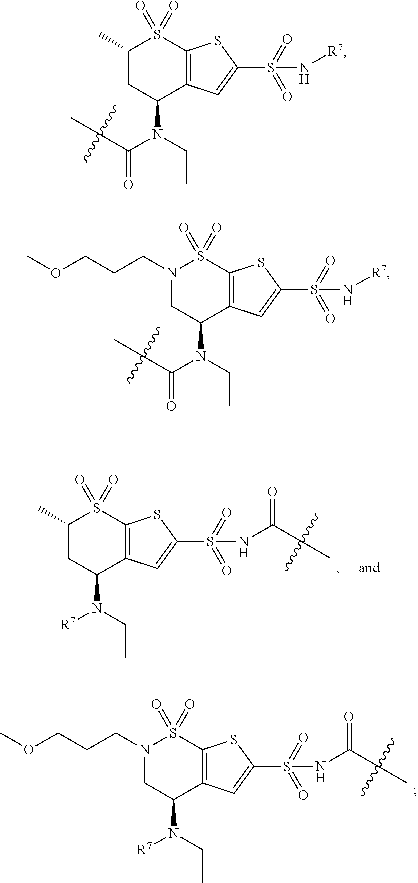
L¹ is selected from 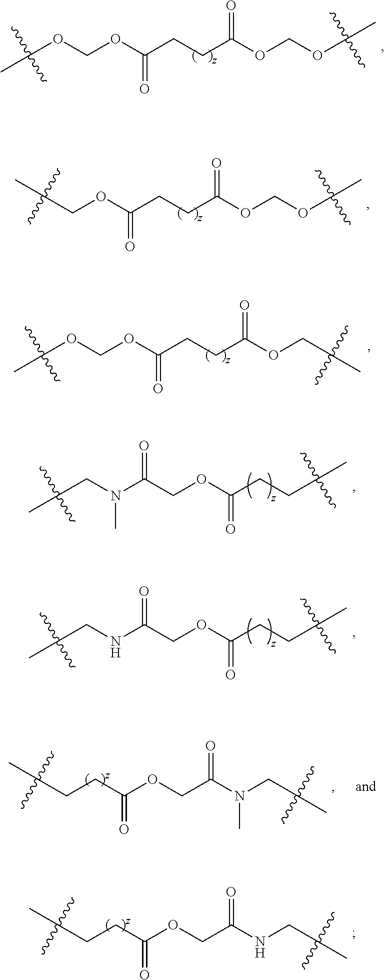
L² is selected from 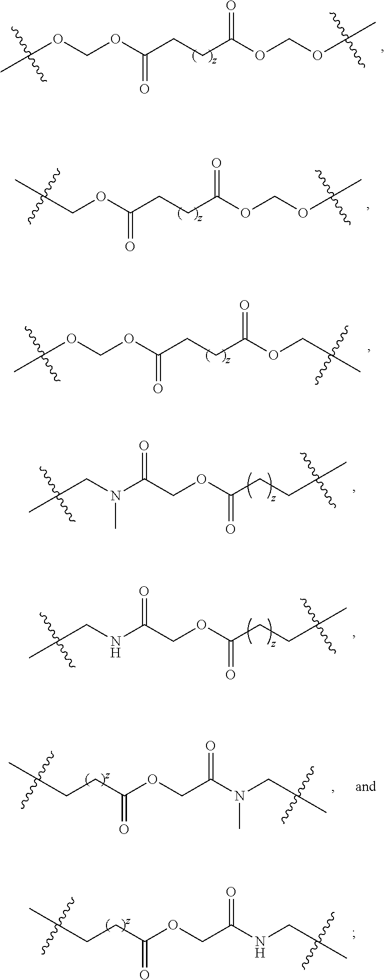
x and y are an integer independently selected from 1, 2, 3, 4, 5, 6, 7, 8, 9, 10, 11, 12, 13, 14, 15, 16, 17, 18, 19, and 20; and
z is an integer selected from 0, 1, 2, 3, 4, 5, 6, 7, 8, 9, or 10.

16. The compound of claim 15, wherein R^{20a} is selected from

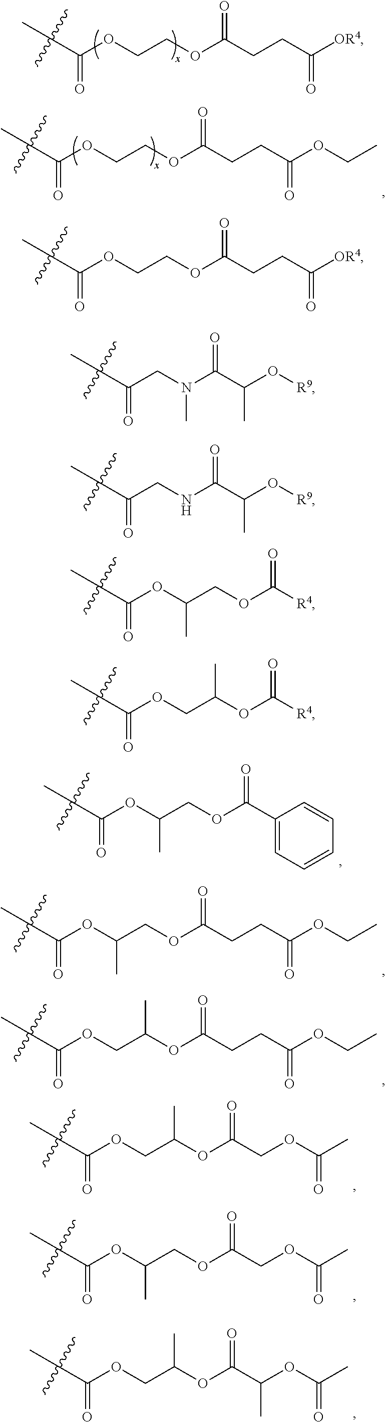

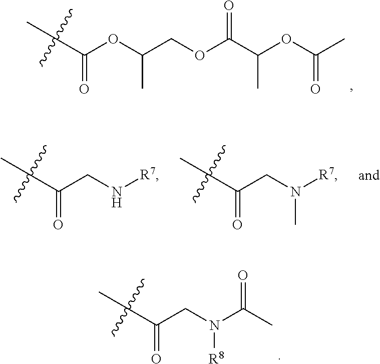

17. The compound of claim 15, wherein L¹ is selected from

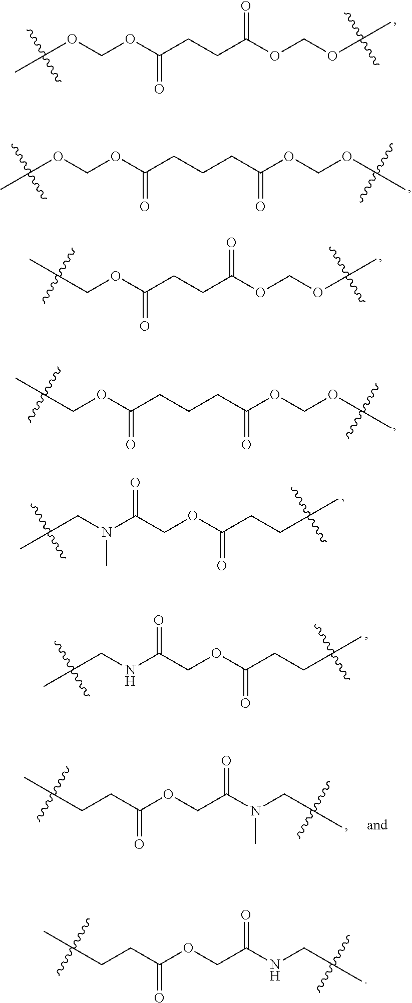

18. The compound of claim 15, wherein R²² is selected from

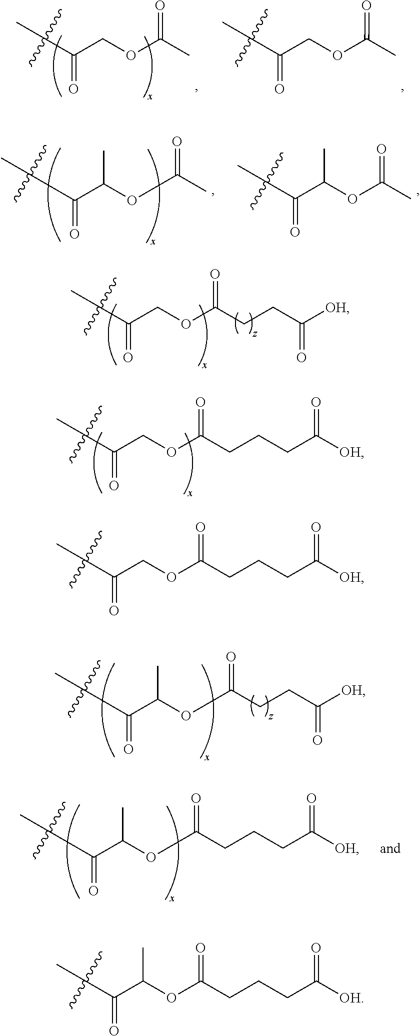

19. The compound of claim 15, wherein z is selected from 0, 1, 2, 3, 4, 5, and 6.

20. The compound of claim 15, wherein z is selected from 0, 1, 2, and 3.

21. The compound of claim 15, wherein xis selected from 1, 2, 3, 4, 5, 6, 7, and 8.

22. The compound of claim 15, wherein xis selected from 1, 2, 3, 4, 5, and 6.

23. Polymeric microparticles comprising an active agent selected from a compound of claim 1 or a pharmaceutically acceptable salt thereof encapsulated in a blend or one or more hydrophobic polymer and an amphiphilic polymer wherein the microparticles release the active agent for at least 1 month.

24. The microparticles of claim 23, wherein the hydrophobic polymer is polylactic acid and/or poly(lactide-co-glycolide).

25. The microparticles of claim 23, wherein the amphiphilic polymer is pegylated hydrophobic polymer.

26. The microparticles of claim 23, wherein the pegylated hydrophobic polymer is PEG conjugated to PLGA.

27. The microparticles of claim 23, wherein the average diameter of the microparticles is greater than 10 μM.

28. A pharmaceutical composition comprising a compound of claim 1 in a pharmaceutically acceptable carrier.

29. A method for the treatment of an ocular disorder in a host in need thereof comprising administering an effective amount of a compound of claim 1 or a pharmaceutically acceptable salt thereof optionally in a pharmaceutically acceptable carrier.

30. The method of claim 29, wherein the disorder is selected from glaucoma, age-related macular degeneration, a disorder related to an increase in intraocular pressure (IOP), a disorder requiring neuroprotection, age-related macular degeneration, and diabetic retinopathy.

31. The method of claim 29, wherein the compound is administered via intravitreal, intrastromal, intracameral, sub-tenon, sub-retinal, retro-bulbar, peribulbar, suprachoroidal, choroidal, subchoroidal, conjunctival, sub conjunctival, episcleral, posterior juxtascleral, circumcorneal, or tear duct injection.

32. The method of claim 29, wherein the host is a human.
